(19) United States
(12) Patent Application Publication
(10) Pub. No.: US 2024/0174647 A1
(43) Pub. Date: May 30, 2024

(54) KINASE MODULATORS AND METHODS OF USE THEREOF

(71) Applicant: NEURON23, INC., South San Francisco, CA (US)

(72) Inventors: Kejia Wu, South San Francisco, CA (US); Abbas Abdoli, South San Francisco, CA (US); Daniel Guay, South San Francisco, CA (US); Abdelkhalek Ben Jamaa, South San Francisco, CA (US); Gyanendra Kumar, South San Francisco, CA (US); Zhonghua Pei, South San Francisco, CA (US); Ryan Simard, South San Francisco, CA (US); Arkadii Vaisburg, South San Francisco, CA (US); Ming Yu, South San Francisco, CA (US)

(21) Appl. No.: 18/375,063

(22) Filed: Sep. 29, 2023

Related U.S. Application Data
(60) Provisional application No. 63/411,846, filed on Sep. 30, 2022.

(51) Int. Cl. C07D 403/12; C07D 401/14; C07D 405/14; C07D 413/14; C07D 471/04; C07D 487/04; C07D 498/04

(52) U.S. Cl. CPC C07D 403/12; C07D 401/14; C07D 405/14; C07D 413/14; C07D 471/04; C07D 487/04; C07D 498/04

(57) ABSTRACT

The invention provides compounds that modulate the activity of protein kinases that are associated with human diseases, disorders, and conditions. In particular, compounds of the invention inhibit LRRK2.

### FIELD OF THE INVENTION

The invention provides compounds that modulate the activity of kinases, such as leucine rich repeat kinase 2 (LRRK2).

### BACKGROUND

A variety of medical conditions that affect millions of people are caused or exacerbated by unregulated activity of protein kinases. For example, aberrant kinase activity is associated with autoimmune diseases, inflammatory diseases, bone diseases, metabolic diseases, neurological and neurodegenerative diseases, cancer, cardiovascular diseases, allergies, asthma, Alzheimer's disease, Parkinson's disease, skin disorders, eye diseases, infectious diseases and hormone-related diseases. For many such disorders, however, no effective inhibitor or activator exists for the particular kinase that causes the disorder or its symptoms. Consequently, patients continue to suffer from an array of disorders due to the lack of a suitable drug for their conditions.

Parkinson's disease (PD) is a neurodegenerative disorder characterized by selective degeneration and cell death of dopaminergic neurons in the substantial nigra region of the brain. Parkinson's disease was generally considered to be sporadic and of unknown etiology. In the past few years, there has been an important development of the understanding of the genetic basis of this disease and associated pathogenic mechanisms. Parkinson's disease is a chronic, progressive motor system disorder that afflicts approximately one out of every 1000 people, with hereditary Parkinson's disease accounting for 5-10% of all of patients. Parkinson's disease is caused by progressive loss of mid-brain dopamine neurons, leaving patients with impaired ability to direct and control their movements. The primary Parkinson's disease symptoms are trembling, rigidity, slowness of movement, and impaired balance. Many Parkinson's disease patients also experience other symptoms such as emotional changes, memory loss, speech problems, and sleeping disorders.

Pathologically, the disease is characterized by loss of dopaminergic neurons with the consequent decrease in dopamine levels in the brain and by aggregation of the protein α-synuclein in the dopaminergic neurons. These aggregations called Lewy-bodies are composed of insoluble α-synuclein phosphorylated at serine-129 and ubiquitin. Current Parkinson's disease therapeutic intervention strategies aim at increasing the dopamine levels in areas innervated by dopaminergic neurons in the brain. Levadopa is a precursor of dopamine, and it is therapeutically used to increase dopamine levels. Carbidopa is an inhibitor of the enzyme aromatic-L-amino-acid decarboxylase also known as DOPA decarboxylase, and it is often co-administered with levadopa to increase the fraction of levadopa which reaches the clinically relevant regions in the brain.

### SUMMARY

Inhibitors of leucine rich repeat kinase 2 (LRRK2) are known to be effective for treatment of Parkinson's Disease (PD). The invention provides LRRK2 inhibitors for treatment of PD.

In an aspect, the invention provides compounds of formula (I):

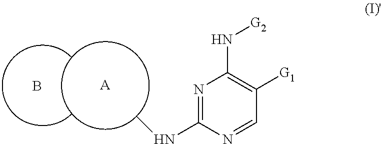

and pharmaceutically acceptable salts thereof,
wherein:
G₁ is CF₃, CHF₂, CH₂F, halogen or cyclopropyl, ethyl, isopropyl;
G₂ is H, substituted or unsubstituted C₁-C₆-alkyl, C₃-C₆-cycloalkyl, C₁-C₆ haloalkyl, C₃-C₆-halocycloalkyl, 1-6 membered heterocycles, heteroaryl, —CH₂-cycloalkyl, —CF₂-cycloalky, —CH(CH₃)-cycloalkyl, —CH₂-aryl, —CH₂-heterocycle, —CH₂-heteroaryl, —CF₂-aryl, and —CH(—CH₃)-aryl;
A and B are independently selected from 5 or 6 membered cycloalkyl or cycloaryl rings comprising one or more heteroatoms and one or more substitutions;
wherein both A and B comprise, cumulatively, at least two (2) nitrogen heteroatoms;
wherein A and B are fused at 2 positions;
wherein the one or more substitutions on the A and B rings are independently selected from: H, halo, C₁-C₆-alkyl, branched alkyl, haloalkyl, substituted or unsubstituted alkenyl, substituted or unsubstituted alkynyl, alkoxy, cycloalkoxy, haloalkoxy, alkoxyalkoxy, cyano, —CH₂-cycloalkyl, —CF₂-cycloalky, —(CH₂)₀₋₂C(CH₃)₂—CN, —(CR₂)₀₋₄—CN, —(CH₂)₀₋₄SO₂R′, —CH(CH₃)-cycloalkyl, —CH₂-aryl, —CF₂-aryl, —CH(—CH₃)-aryl, —C(═O)-alkyl, —C(═O)cycloalkyl, —C(═O)—NR′R″, —C(═O)—NH-alkyl, —C(═O)NH₂, hydroxy, —COOH (and ester thereof), alkylsulfonyl, cycloalkylsulfonyl, arylsulfonyl, —S(═O)₂—NR′R″, —NH₂, —NR′R″, —NR′—S(═O)R″, cyanoalkyl, haloalkyl, substituted or unsubstituted alkylsulfonyl, arylsulfonyl, —C(═O)-morpholine, —C(═O)-heterocycles, —C(H)₀₋₁(—CH₃)₁₋₂—OH, —CH₂—C(═O)—NH₂; 3-6 membered heterocycle, any of which may have one or more substituents, wherein the 3-6 membered heterocycle comprises at least one heteroatom independently selected from O, S, and N;
wherein R′ and R″ are independently selected from a group consisting of H, alkyl, substituted or unsubstituted aryl, and heterocycles.

In certain embodiments, the compound of the invention is a compound of Formula (II):

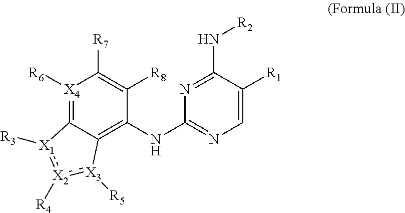

wherein,
X₁, X₂, X₃, and X₄ are C or N, wherein X₁, X₂, and X₃ include either one (1) N, two (2) N, or three (3) N;
R₁ is CF₃, CHF₂, CH₂F, halogen, cyclopropyl, ethyl, isopropyl;
R₂ is H, substituted or unsubstituted C₁-C₆ alkyl, C₃-C₆-cycloalkyl, C₃-C₆-halocycloalkyl; branched alkyl, alkenyl, alkynyl, haloalkyl, alkoxy, cycloalkoxy, haloalkoxy, —CH₂-cycloalkyl, —CF₂-cycloalky, tetrahydrofuran, —CH(—CH₃)-cycloalkyl, —CH₂-aryl, —CF₂-aryl, —CH(—CH)-aryl, —C(═O)-alkyl, —C(═O)cycloalkyl, —C(═O)—NH-alkyl;
R₃, R₄, R₅ are independently selected from a group consisting of: H, substituted or unsubstituted C₁-C₆ alkyl, C₃-C₆-cycloalkyl, C₃-C₆-halocycloalkyl; branched alkyl, alkenyl, alkynyl, haloalkyl, alkoxy, cycloalkoxy, haloalkoxy, nitro, cyano, alkylcyano, —CH₂-cycloalkyl, —CF₂-cycloalky, —CH(CH₃)-cycloalkyl, —CH₂-aryl, —CF₂-aryl, —CH(—CH₃)-aryl, —C(═O)—R′, —C(═O)—NR′R″, —S═(O)₂—R′, —CH₂CN, —C—(CH₃)₂—CN, —C(—CH₃)₂—OH, —CH₂—C(═O)—NH₂;
R₆, R₇, and R₈ are independently selected from a group consisting of: H, halo, C₁-C₆-alkyl, branched alkyl, alkenyl, alkynyl, haloalkyl, alkoxy, cycloalkoxy, haloalkoxy, nitro, cyano, —CH₂-cycloalkyl, —CF₂-cycloalky, —CH(CH₃)-cycloalkyl, —CH₂-aryl, —CF₂-aryl, —CH(—CH₃)-aryl, —C(═O)-alkyl, —C(═O)cycloalkyl, —C(═O)—NH-alkyl, —C(═O)NH₂, hydroxy, —COOH (and ester thereof), —C(═O)-morpholine, —C(═O)-heterocycle, —S═(O)₂—R′, amino, —NR′R″, —NR′S═(O)₂—R′, cyanoalkyl, haloalkyl, —C(—CH₃)₂—OH, —CH₂—C(═O)—NH—; 3-6 membered heterocycle, any of which may have one or more substituents, wherein the 3-6 membered heterocycle comprises at least one heteroatom independently selected from O, S, and N; —S═(O)₂-cyclopropyl, —C(═O)-morpholine, —C(—CH₃)₂—OH, —CH₂—C(═O)—NIH, —CF₃, —OCF₃, tetrahydropyran, 3H-pyran, 2H-pyran, piperidine, alkyl-morpholine;
wherein R′ and R″ are independently selected from a group consisting of H, unsubstituted or substituted C₁-C₅ alkyl, substituted or unsubstituted C₁-C₈ aryl, substituted or unsubstituted C₁-C₈ cycloalkyl or heterocycloalkyl;
wherein substitutions R₃, R₄, R₅, and R₆ are only present where valency permits; and
wherein R₃ and R₆ optionally form a 5-7 membered heteroalkyl ring.

In certain embodiments, R₁ is CF₃.

In certain embodiments, R₁ is CHF₂.

In certain embodiments, wherein R₁ is chloro.

In certain embodiments, wherein R₂ is K, methyl, ethyl, cyclopropyl, halogentated cyclopropyl, ethyl, cyclopentyl, tetrahydrofuran, cyclobutyl, hydroxycyclobutyl, halocyclopropyl, fluorocycopropyl, diflurocyclobutyl, isopropyl, —CH₂CF₃, —CH₂—CH₂—F, —CH₂—CH₂—OH, —CH₂—CH₂—O—CH₃, —CH₂—CH₂S═(O)₂—CH₃, —CH₂-cyclopropyl, —N(CH₃)₂, —CH(CH₃), cyclobutyl, or —CH(CH₃)cyclopropyl.

In certain embodiments, wherein R₃, R₄, R₅ are independently selected from a group consisting of: H, —CH₃, —CH₂—CN, —CH₂—CH₂—O—CH₃, —CH₂—CHF₂, isopropyl, cyanoisopropyl, cyclopropyl, t-butyl, —CH₂-cyclopropyl, —CH₂—C(CH₃)₂—CN, —S═(O)₂—CH₃, —CH₂—CH₂S═(O)₂—CH₃, —C—(CH₃)₂—CN, —S═(O)₂—CH—(CH₃)₂, —S═(O)₂-cyclopropyl, —CH₂—CH₂—S═(O)₂—CH₂—CH₃, —C—(CH₃)₂—S═(O)—CH—(CH₃)₂, —S═(O)₂—CH₂—CH₂—OCH₃, -morpholine, —CH₂-oxetane, C(═O)-morpholine, —C(—CH₃)₂—OH, —CH₂—C(═O)—NH₂.

In certain embodiments, wherein R₆, R₇, and R₈ are independently selected from a group consisting of: H, fluoro, methyl, chloro, cyano, —O—CH₃, CH₃, —CH—(CH₃)₂, —CH₂—CN, —CH₂—CHF₂, —S═(O)₂—CH₃, —C—(CH₃)₂—CN, —S═(O)₂-cyclopropyl, —C(═O)-morpholine, —C(—CH₃)₂—OH, —CH₂—C(═O)—NH₂, —CF₃, —OCF₃, tetrahydropyran, 3H-pyran, 2H-pyran, piperidine, methylpiperidine, alkyl-morpholine.

In certain the embodiments, R₁ and R₂ together form a ring.

In certain embodiments, R₃ and R₆ together form a fused ring.

In certain embodiments, R₃ and R₆ together form a morpholine ring

In certain embodiments, X₁ and X₂ are N, and X₃ is C.

In another aspect, the compound of the invention is a compound of Formula (III):

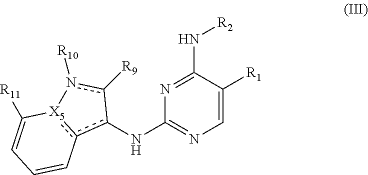

wherein, X₅ is selected from C or N;
R₉, R₁₀, or R₁₁ are independently selected from a group consisting of: H, substituted or unsubstituted C₁-C₆ alkyl, C₁-C₆-cycloalkyl, C₃-C₅-halocycloalkyl; C₁-C₆-alkyl, branched alkyl, alkenyl, alkynyl, haloalkyl, alkoxy, cycloalkoxy, haloalkoxy, nitro, cyano, —CH₂-cycloalkyl, —CF₂-cycloalky, —CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —S═(O)₂—CH₃, —C—(CH₃)₂—CN, —S═(O)₂-cyclopropyl, —C(═O)-morpholine, —C(—CH₃)₂—OH, —CH₂—C(═O)—NH₂;
wherein substitutions R₉, R₁, and R are only present where valency permits.

In certain embodiments, X₅ is C.

In certain embodiments, X₅ is N.

In certain embodiments, R₉, R₁₀, or R₁₁ are independently selected from a group consisting of: H, fluoro, chloro, cyano, CH₃, —O—CH₃, —CH—(CH₃)₂, or —CH₂—CN.

In another aspect, the compound is a compound of Formula (IV):

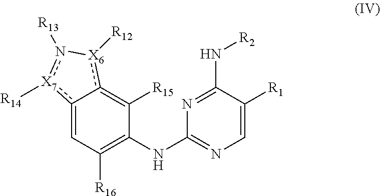

wherein, X₆ and X₇ are selected from C or N, wherein at least one of X₆ and X₇ is N;
R₁₂, R₁₃, and R₁₄ are independently selected from a group consisting of: H, substituted or unsubstituted C₁-C₆ alkyl, C₃-C₆-cycloalkyl, C₃-C₆-halocycloalkyl; C₁-C₆ branched alkyl, C₁-C₆-alkenyl, C₁-C₆-alkynyl, C₁-C₆-haloalkyl, C₁-C₆-alkoxy, cycloalkoxy, haloalkoxy, halo, nitro, cyano, —CH₂-cycloalkyl, —CF₂-cycloalky, —CH(CH₃)-cycloalkyl, —CH₂-aryl, —CF₂-aryl, —CH(—CH₃)-aryl, C(═O)-alky, —C(═O)heterocycloalkyl, and —C(═O)-morpholine;
R₁₅ and R₁₆ are independently selected from a group consisting of: H, halo, substituted or unsubstituted C₁-C₆ alkyl, alkyloxy, cyanoalkyl, C₁-C₆-branched alkyl, alkenyl, alkynyl, haloalkyl, alkoxy, cycloalkoxy, haloalkoxy, halo, nitro, cyano, —CH₂-cycloalkyl, —CF₂-cycloalky, —CH(CH₃)-cycloalkyl, —C(═O)heterocycloalkyl, and —C(═O)-morpholine;
wherein substitutions R₁₂, R₁₃, and R₁₄ are only present where valency permits.

In certain embodiments, X₆ in N and X₇ is C.

In certain embodiments, X₆ in C and X₇ is N.

In certain embodiments, R₁₂, R₁₃, and R₁₄ are independently selected from a group consisting of: H, fluoro, chloro, bromo, cyano, CH₃, —CH—(CH₃)₂, —CH₂—CN and —C(═O)-morpholine.

In certain embodiments, R₁₅ and R₁₆ are independently selected from a group consisting of: H, fluoro, chloro, bromo, CH₃, —O—CH₃, —O—CF₃, —CF₃, ethyl, vinyl, or —CH₂—CN.

In another aspect, the compound of the invention is a compound of Formula (V):

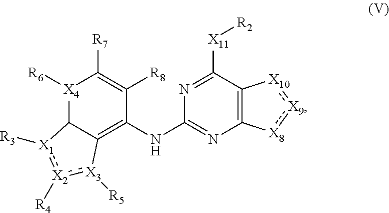

wherein X₈ is N or NH;
X₉ is CH or N,
X₁₀ is NR₁₇, N, CH, or CR₁₇;
X₁₁ is O or NH.
R₁₇ is selected from the group consisting of H, halo, and alkyl.

In certain embodiments, at least one of X₈, X₉, and X₁₀ is CH.

In certain embodiments, R₁₇ is chloro.

In certain embodiments, R₁₇ is methyl.

In certain embodiments, the compound of the invention is selected from:

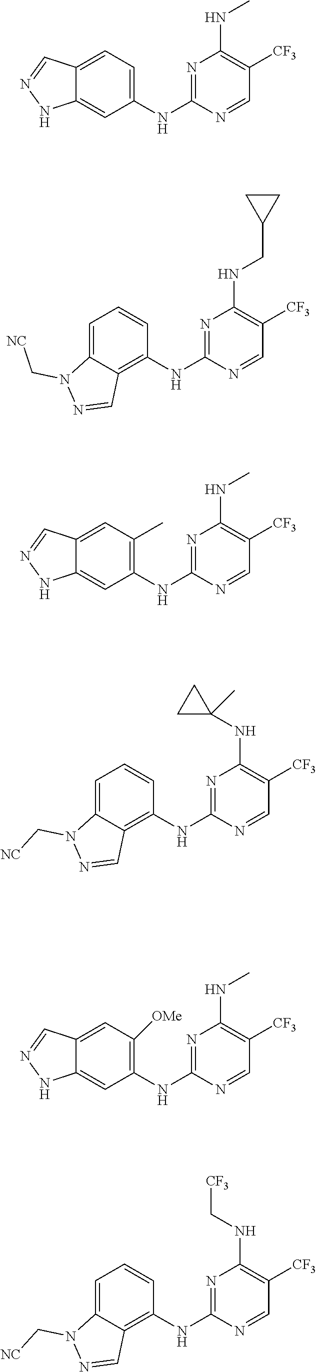 [SMILES: CNc1nc(Nc2ccc3cn[nH]c3c2)ncc1C(F)(F)F ; N#CCn1ncc2c(Nc3ncc(C(F)(F)F)c(NCC4CC4)n3)cccc21 ; CNc1nc(Nc2cc3[nH]ncc3cc2C)ncc1C(F)(F)F ; CC1(Nc2nc(Nc3cccc4c3cnn4CC#N)ncc2C(F)(F)F)CC1 ; CNc1nc(Nc2cc3[nH]ncc3cc2OC)ncc1C(F)(F)F ; N#CCn1ncc2c(Nc3ncc(C(F)(F)F)c(NCC(F)(F)F)n3)cccc21]

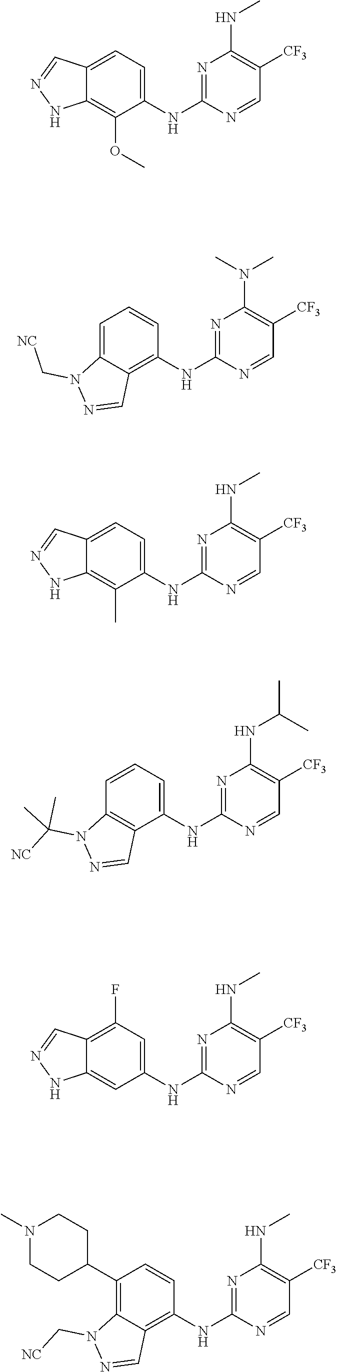 [SMILES: CNc1nc(Nc2ccc3cn[nH]c3c2OC)ncc1C(F)(F)F ; CN(C)c1nc(Nc2cccc3c2cnn3CC#N)ncc1C(F)(F)F ; CNc1nc(Nc2ccc3cn[nH]c3c2C)ncc1C(F)(F)F ; CC(C)Nc1nc(Nc2cccc3c2cnn3C(C)(C)C#N)ncc1C(F)(F)F ; CNc1nc(Nc2cc(F)c3cn[nH]c3c2)ncc1C(F)(F)F ; CNc1nc(Nc2ccc(C3CCN(C)CC3)c3c2cnn3CC#N)ncc1C(F)(F)F]

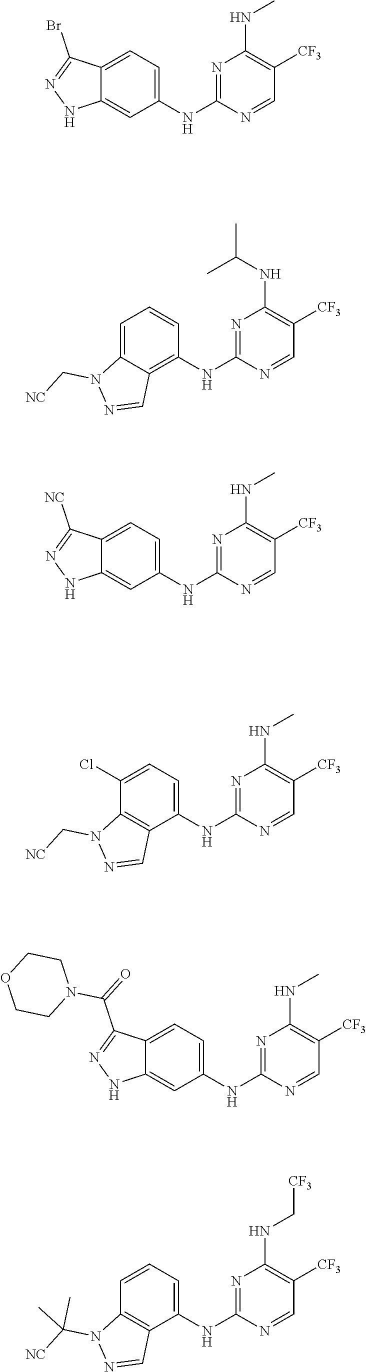 [SMILES: CNc1nc(Nc2ccc3c(Br)n[nH]c3c2)ncc1C(F)(F)F ; CC(C)Nc1nc(Nc2cccc3c2cnn3CC#N)ncc1C(F)(F)F ; CNc1nc(Nc2ccc3c(C#N)n[nH]c3c2)ncc1C(F)(F)F ; CNc1nc(Nc2ccc(Cl)c3c2cnn3CC#N)ncc1C(F)(F)F ; CNc1nc(Nc2ccc3c(C(=O)N4CCOCC4)n[nH]c3c2)ncc1C(F)(F)F ; CC(C)(C#N)n1ncc2c(Nc3ncc(C(F)(F)F)c(NCC(F)(F)F)n3)cccc21]

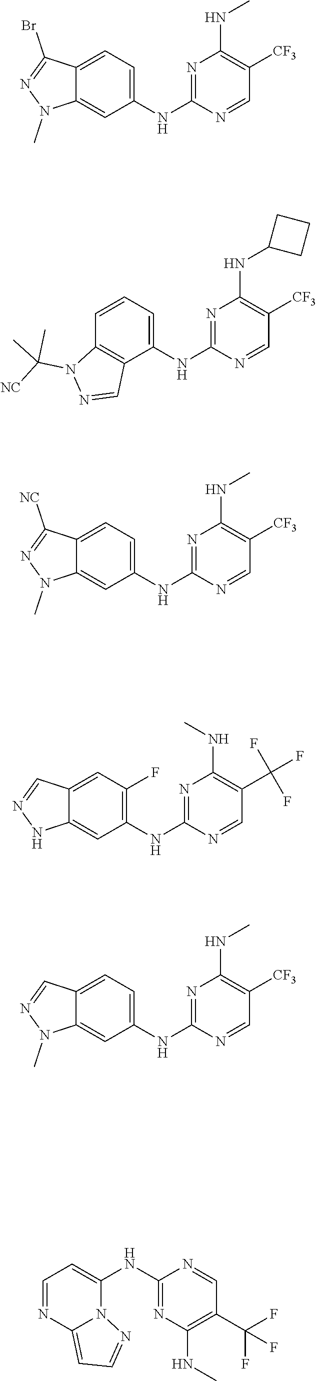 [SMILES: CNc1nc(Nc2ccc3c(Br)nn(C)c3c2)ncc1C(F)(F)F ; CC(C)(C#N)n1ncc2c(Nc3ncc(C(F)(F)F)c(NC4CCC4)n3)cccc21 ; CNc1nc(Nc2ccc3c(C#N)nn(C)c3c2)ncc1C(F)(F)F ; CNc1nc(Nc2cc3[nH]ncc3cc2F)ncc1C(F)(F)F ; CNc1nc(Nc2ccc3cnn(C)c3c2)ncc1C(F)(F)F ; CNc1nc(Nc2ccnc3ccnn23)ncc1C(F)(F)F]

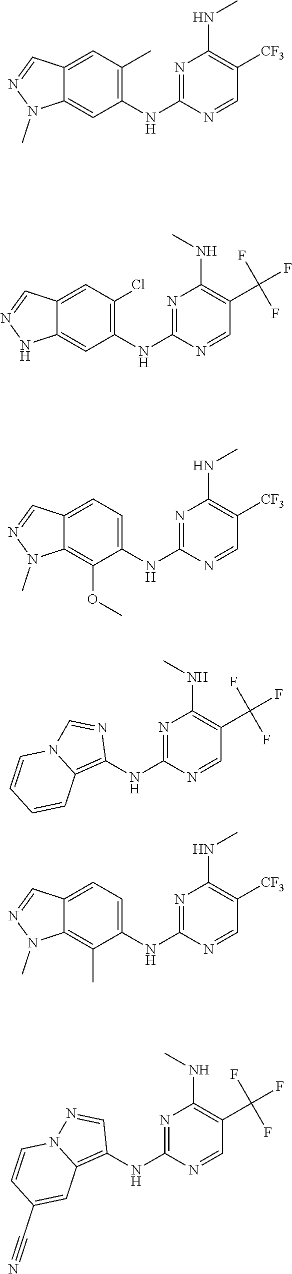 [SMILES: CNc1nc(Nc2cc3c(cnn3C)cc2C)ncc1C(F)(F)F ; CNc1nc(Nc2cc3[nH]ncc3cc2Cl)ncc1C(F)(F)F ; CNc1nc(Nc2ccc3cnn(C)c3c2OC)ncc1C(F)(F)F ; CNc1nc(Nc2ncn3ccccc23)ncc1C(F)(F)F ; CNc1nc(Nc2ccc3cnn(C)c3c2C)ncc1C(F)(F)F ; CNc1nc(Nc2cnn3ccc(C#N)cc23)ncc1C(F)(F)F]

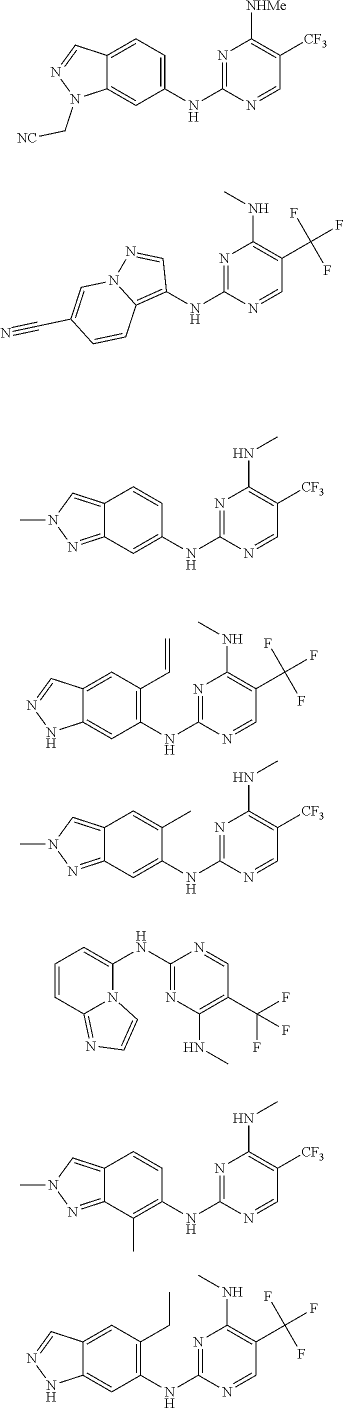

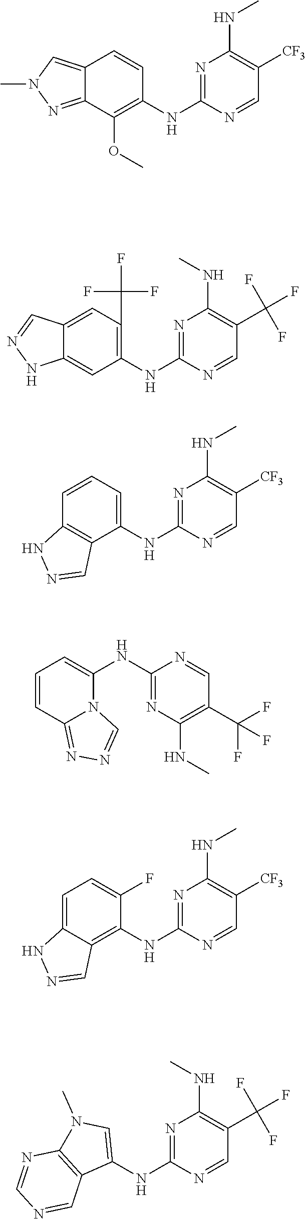 [SMILES: CNc1nc(Nc2ccc3cn(C)nc3c2OC)ncc1C(F)(F)F ; CNc1nc(Nc2cc3[nH]ncc3cc2C(F)(F)F)ncc1C(F)(F)F ; CNc1nc(Nc2cccc3[nH]ncc23)ncc1C(F)(F)F ; CNc1nc(Nc2cccc3nncn23)ncc1C(F)(F)F ; CNc1nc(Nc2c(F)ccc3[nH]ncc23)ncc1C(F)(F)F ; CNc1nc(Nc2cn(C)c3ncncc23)ncc1C(F)(F)F]

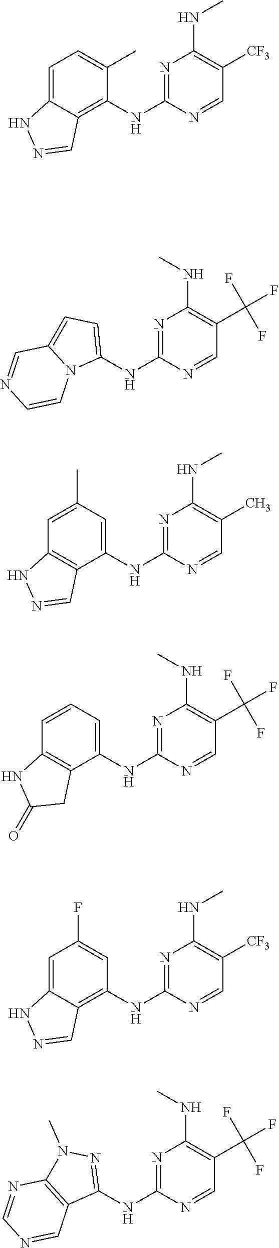 [SMILES: CNc1nc(Nc2c(C)ccc3[nH]ncc23)ncc1C(F)(F)F ; CNc1nc(Nc2ccc3cnccn23)ncc1C(F)(F)F ; CNc1nc(Nc2cc(C)cc3[nH]ncc23)ncc1C ; CNc1nc(Nc2cccc3c2CC(=O)N3)ncc1C(F)(F)F ; CNc1nc(Nc2cc(F)cc3[nH]ncc23)ncc1C(F)(F)F ; CNc1nc(Nc2nn(C)c3ncncc23)ncc1C(F)(F)F]

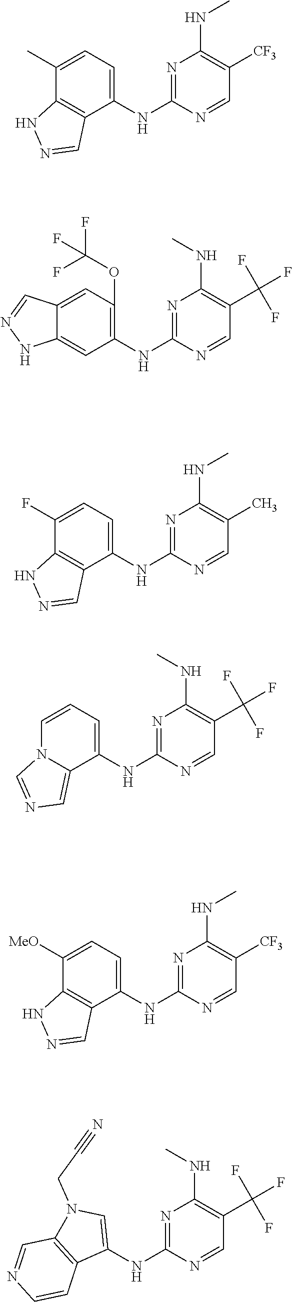 [SMILES: CNc1nc(Nc2ccc(C)c3[nH]ncc23)ncc1C(F)(F)F ; CNc1nc(Nc2cc3[nH]ncc3cc2OC(F)(F)F)ncc1C(F)(F)F ; CNc1nc(Nc2ccc(F)c3[nH]ncc23)ncc1C ; CNc1nc(Nc2cccn3cncc23)ncc1C(F)(F)F ; CNc1nc(Nc2ccc(OC)c3[nH]ncc23)ncc1C(F)(F)F ; CNc1nc(Nc2cn(CC#N)c3cnccc23)ncc1C(F)(F)F]

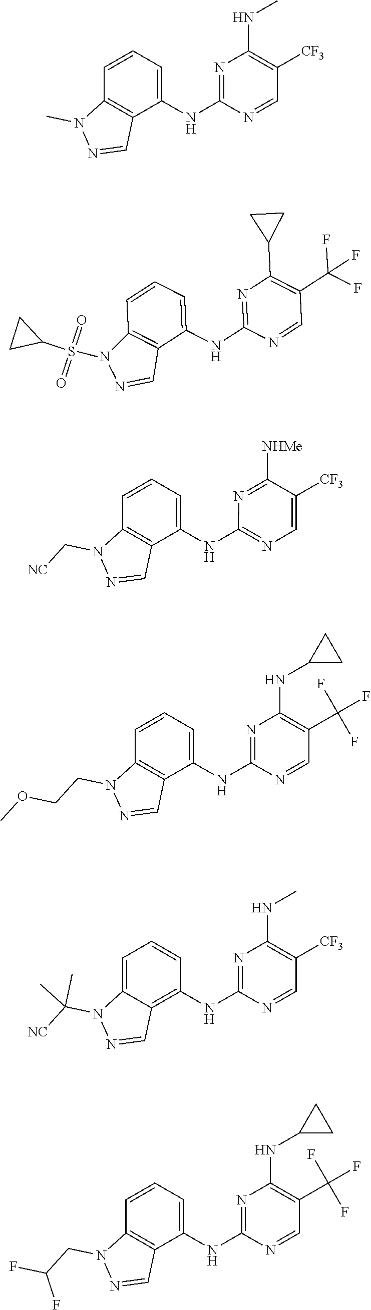 [SMILES: CNc1nc(Nc2cccc3c2cnn3C)ncc1C(F)(F)F ; O=S(=O)(C1CC1)n1ncc2c(Nc3ncc(C(F)(F)F)c(C4CC4)n3)cccc21 ; CNc1nc(Nc2cccc3c2cnn3CC#N)ncc1C(F)(F)F ; COCCn1ncc2c(Nc3ncc(C(F)(F)F)c(NC4CC4)n3)cccc21 ; CNc1nc(Nc2cccc3c2cnn3C(C)(C)C#N)ncc1C(F)(F)F ; FC(F)Cn1ncc2c(Nc3ncc(C(F)(F)F)c(NC4CC4)n3)cccc21]

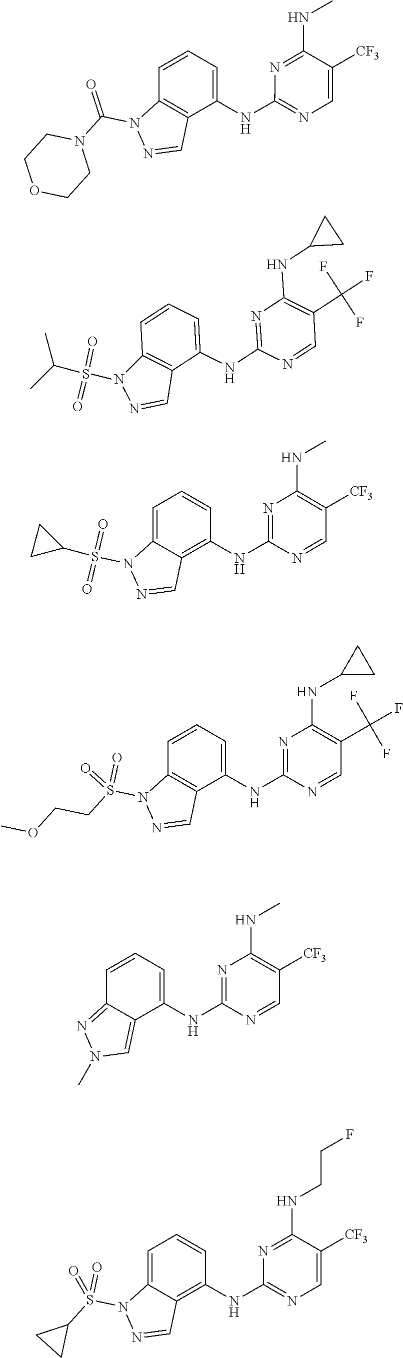 [SMILES: CNc1nc(Nc2cccc3c2cnn3C(=O)N2CCOCC2)ncc1C(F)(F)F ; CC(C)S(=O)(=O)n1ncc2c(Nc3ncc(C(F)(F)F)c(NC4CC4)n3)cccc21 ; CNc1nc(Nc2cccc3c2cnn3S(=O)(=O)C2CC2)ncc1C(F)(F)F ; COCCS(=O)(=O)n1ncc2c(Nc3ncc(C(F)(F)F)c(NC4CC4)n3)cccc21 ; CNc1nc(Nc2cccc3nn(C)cc23)ncc1C(F)(F)F ; O=S(=O)(C1CC1)n1ncc2c(Nc3ncc(C(F)(F)F)c(NCCF)n3)cccc21]

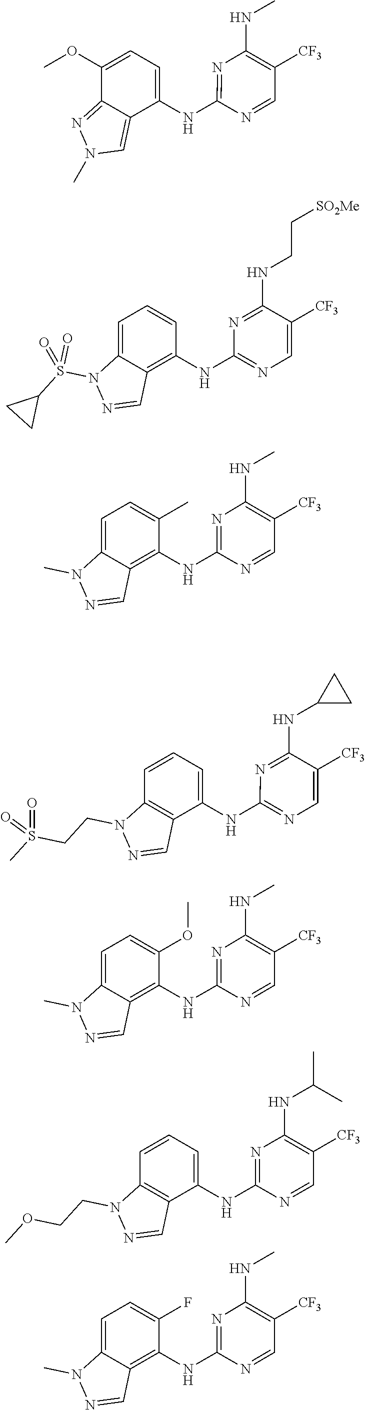

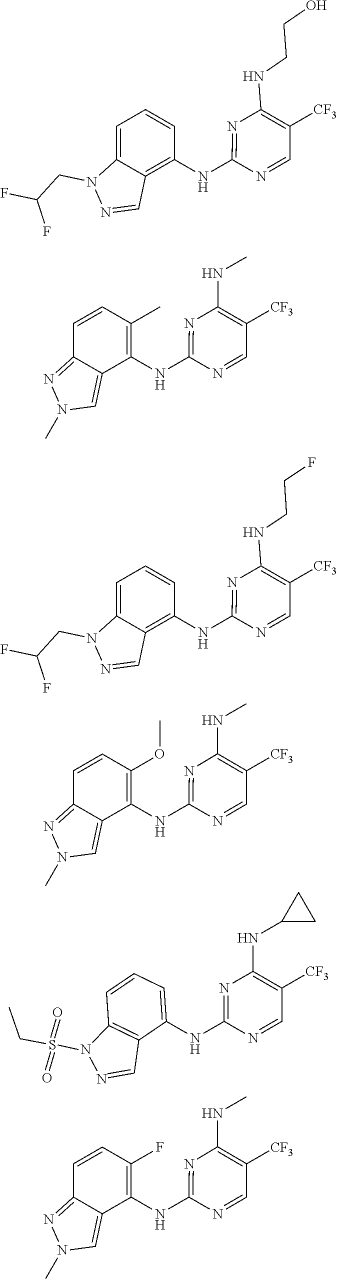 [SMILES: OCCNc1nc(Nc2cccc3c2cnn3CC(F)F)ncc1C(F)(F)F ; CNc1nc(Nc2c(C)ccc3nn(C)cc23)ncc1C(F)(F)F ; FCCNc1nc(Nc2cccc3c2cnn3CC(F)F)ncc1C(F)(F)F ; CNc1nc(Nc2c(OC)ccc3nn(C)cc23)ncc1C(F)(F)F ; CCS(=O)(=O)n1ncc2c(Nc3ncc(C(F)(F)F)c(NC4CC4)n3)cccc21 ; CNc1nc(Nc2c(F)ccc3nn(C)cc23)ncc1C(F)(F)F]

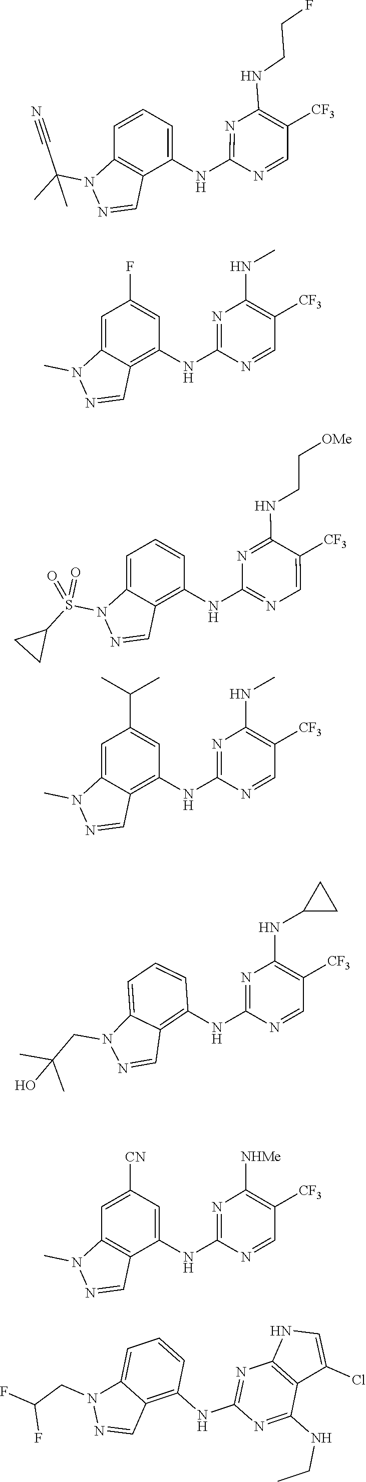

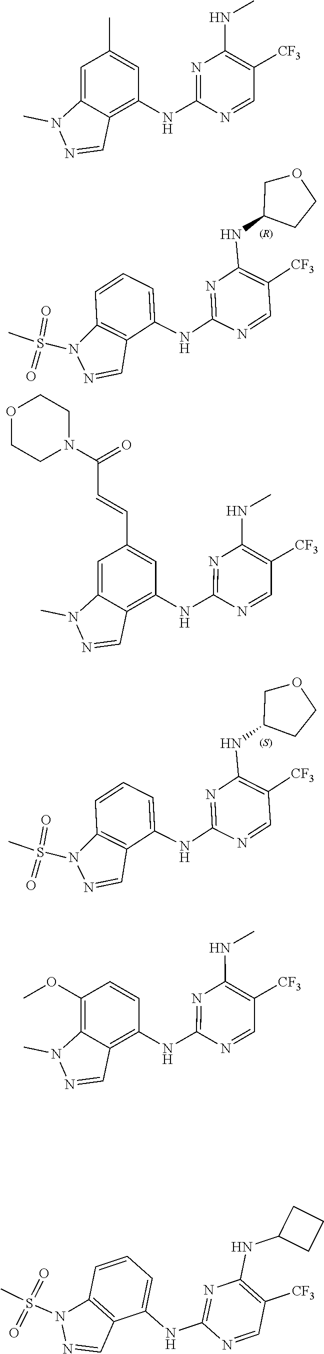

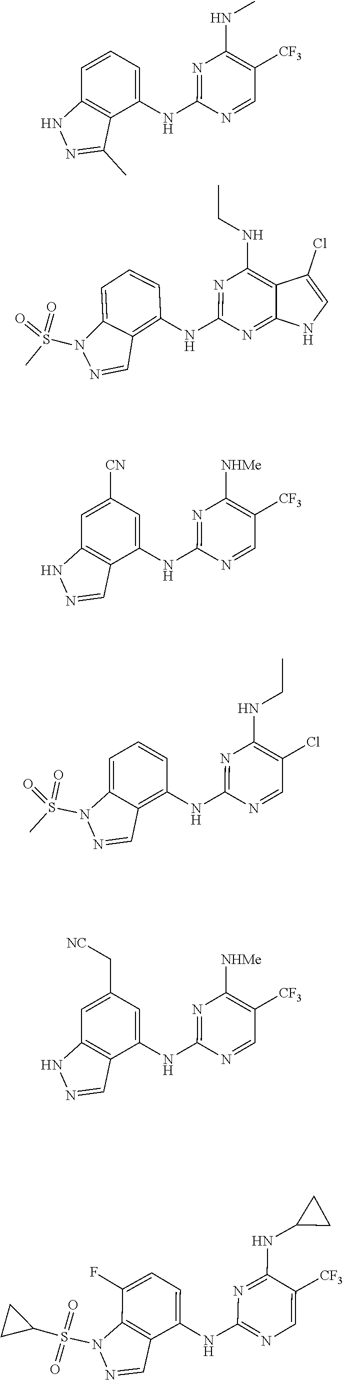 [SMILES: CNc1nc(Nc2cccc3[nH]nc(C)c23)ncc1C(F)(F)F ; CCNc1nc(Nc2cccc3c2cnn3S(C)(=O)=O)nc2[nH]cc(Cl)c12 ; CNc1nc(Nc2cc(C#N)cc3[nH]ncc23)ncc1C(F)(F)F ; CCNc1nc(Nc2cccc3c2cnn3S(C)(=O)=O)ncc1Cl ; CNc1nc(Nc2cc(CC#N)cc3[nH]ncc23)ncc1C(F)(F)F ; O=S(=O)(C1CC1)n1ncc2c(Nc3ncc(C(F)(F)F)c(NC4CC4)n3)ccc(F)c21]

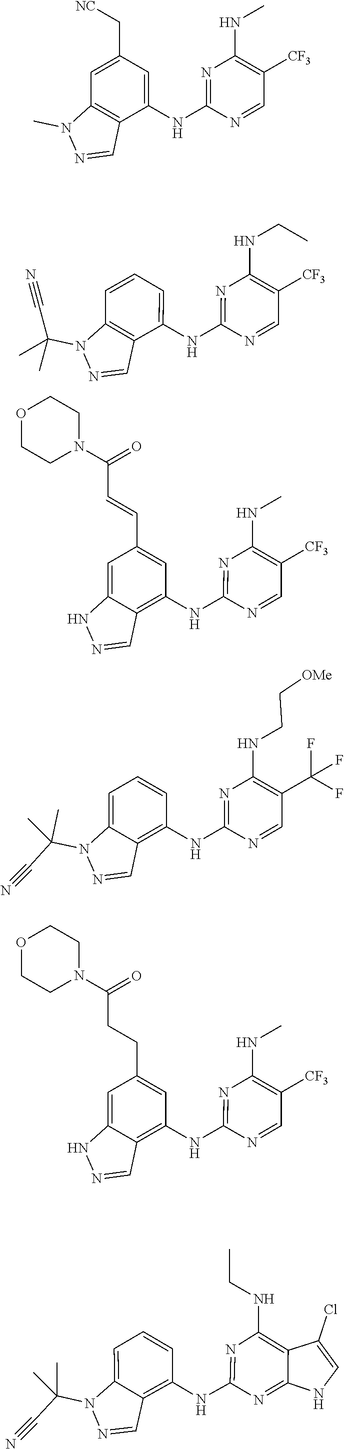

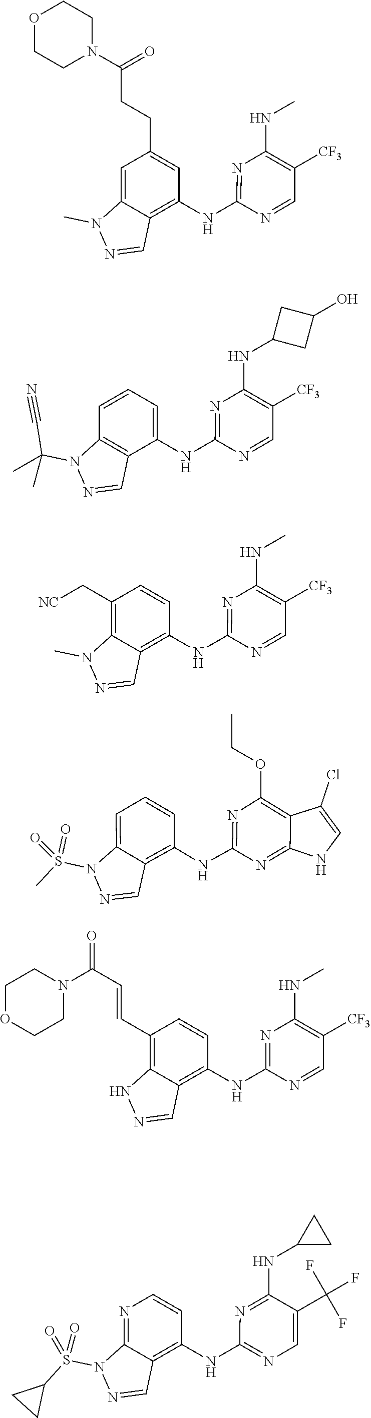

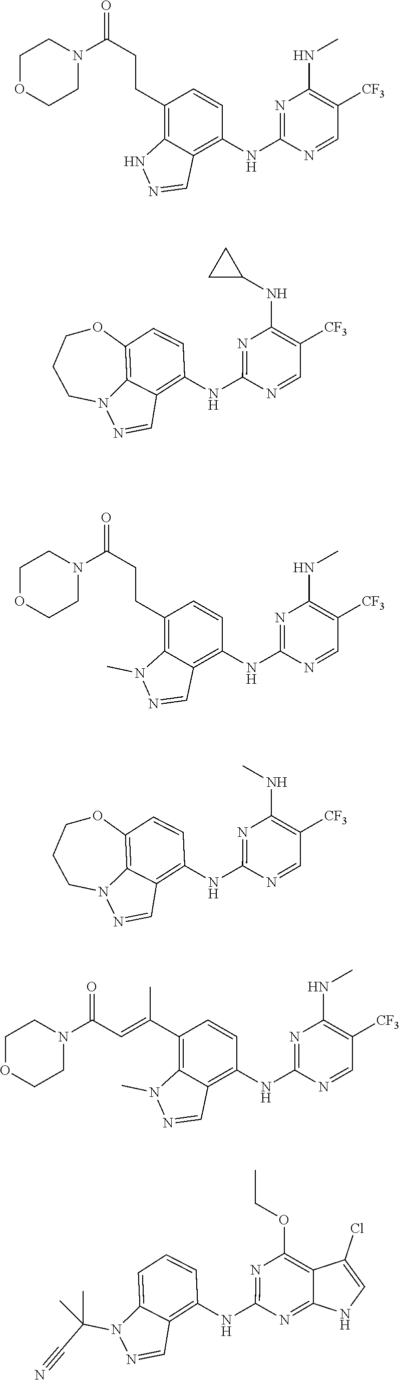 [SMILES: CNc1nc(Nc2ccc(CCC(=O)N3CCOCC3)c3[nH]ncc23)ncc1C(F)(F)F ; FC(F)(F)c1cnc(Nc2ccc3c4c2cnn4CCCO3)nc1NC1CC1 ; CNc1nc(Nc2ccc(CCC(=O)N3CCOCC3)c3c2cnn3C)ncc1C(F)(F)F ; CNc1nc(Nc2ccc3c4c2cnn4CCCO3)ncc1C(F)(F)F ; CNc1nc(Nc2ccc(/C(C)=C/C(=O)N3CCOCC3)c3c2cnn3C)ncc1C(F)(F)F ; CCOc1nc(Nc2cccc3c2cnn3C(C)(C)C#N)nc2[nH]cc(Cl)c12]

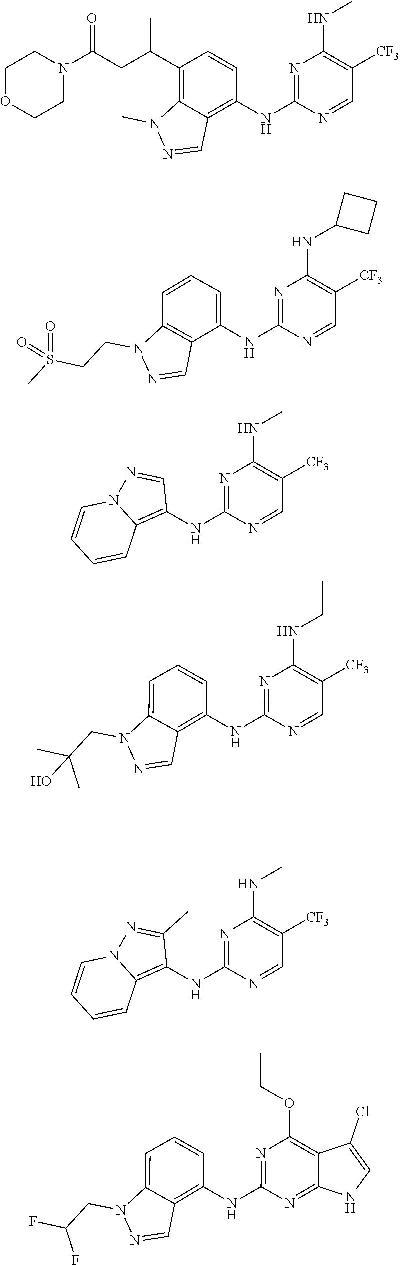 [SMILES: CNc1nc(Nc2ccc(C(C)CC(=O)N3CCOCC3)c3c2cnn3C)ncc1C(F)(F)F ; CS(=O)(=O)CCn1ncc2c(Nc3ncc(C(F)(F)F)c(NC4CCC4)n3)cccc21 ; CNc1nc(Nc2cnn3ccccc23)ncc1C(F)(F)F ; CCNc1nc(Nc2cccc3c2cnn3CC(C)(C)O)ncc1C(F)(F)F ; CNc1nc(Nc2c(C)nn3ccccc23)ncc1C(F)(F)F ; CCOc1nc(Nc2cccc3c2cnn3CC(F)F)nc2[nH]cc(Cl)c12]

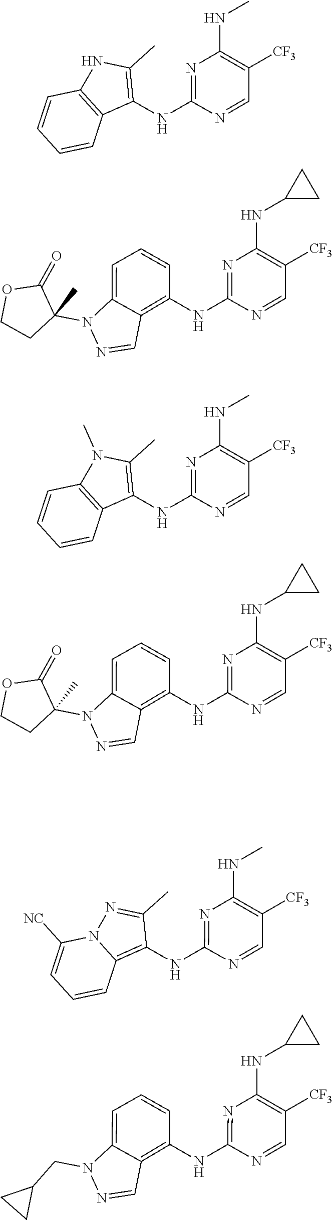 [SMILES: CNc1nc(Nc2c(C)[nH]c3ccccc23)ncc1C(F)(F)F ; C[C@@]1(n2ncc3c(Nc4ncc(C(F)(F)F)c(NC5CC5)n4)cccc32)CCOC1=O ; CNc1nc(Nc2c(C)n(C)c3ccccc23)ncc1C(F)(F)F ; C[C@]1(n2ncc3c(Nc4ncc(C(F)(F)F)c(NC5CC5)n4)cccc32)CCOC1=O ; CNc1nc(Nc2c(C)nn3c(C#N)cccc23)ncc1C(F)(F)F ; FC(F)(F)c1cnc(Nc2cccc3c2cnn3CC2CC2)nc1NC1CC1]

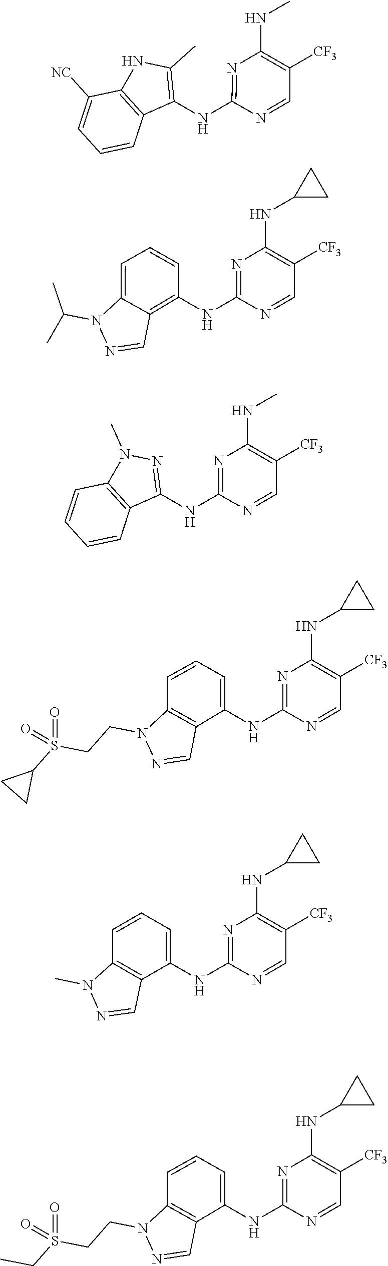 [SMILES: CNc1nc(Nc2c(C)[nH]c3c(C#N)cccc23)ncc1C(F)(F)F ; CC(C)n1ncc2c(Nc3ncc(C(F)(F)F)c(NC4CC4)n3)cccc21 ; CNc1nc(Nc2nn(C)c3ccccc23)ncc1C(F)(F)F ; O=S(=O)(CCn1ncc2c(Nc3ncc(C(F)(F)F)c(NC4CC4)n3)cccc21)C1CC1 ; Cn1ncc2c(Nc3ncc(C(F)(F)F)c(NC4CC4)n3)cccc21 ; CCS(=O)(=O)CCn1ncc2c(Nc3ncc(C(F)(F)F)c(NC4CC4)n3)cccc21]

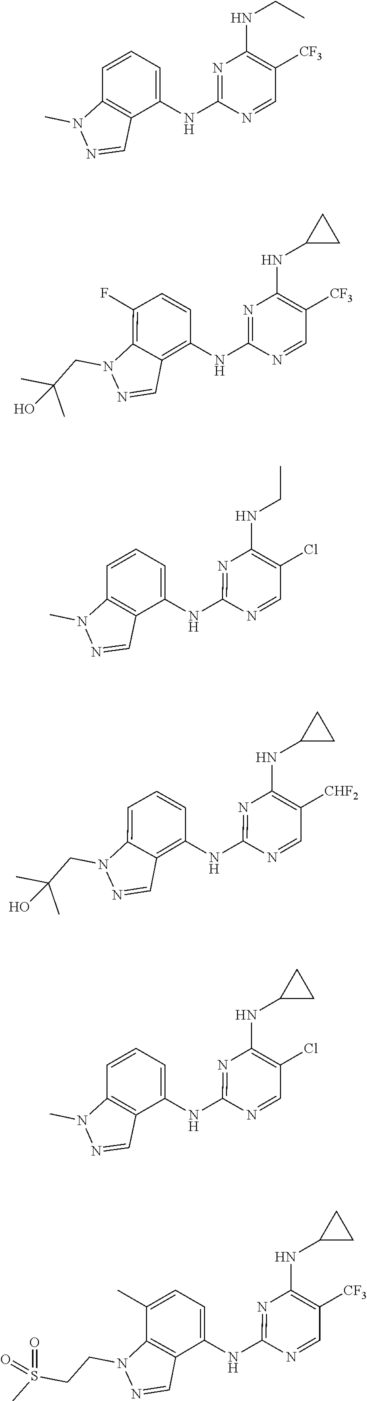 [SMILES: CCNc1nc(Nc2cccc3c2cnn3C)ncc1C(F)(F)F ; CC(C)(O)Cn1ncc2c(Nc3ncc(C(F)(F)F)c(NC4CC4)n3)ccc(F)c21 ; CCNc1nc(Nc2cccc3c2cnn3C)ncc1Cl ; CC(C)(O)Cn1ncc2c(Nc3ncc(C(F)F)c(NC4CC4)n3)cccc21 ; Cn1ncc2c(Nc3ncc(Cl)c(NC4CC4)n3)cccc21 ; Cc1ccc(Nc2ncc(C(F)(F)F)c(NC3CC3)n2)c2cnn(CCS(C)(=O)=O)c12]

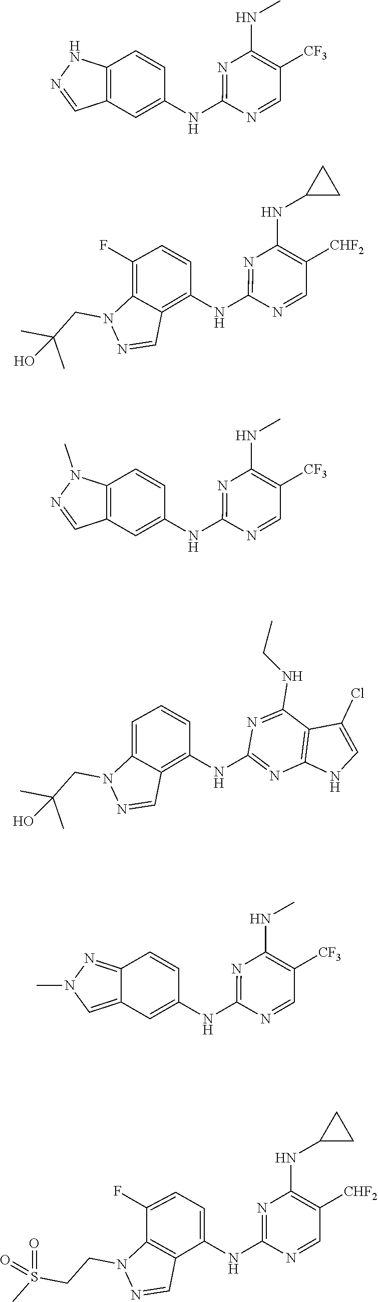 [SMILES: CNc1nc(Nc2ccc3[nH]ncc3c2)ncc1C(F)(F)F ; CC(C)(O)Cn1ncc2c(Nc3ncc(C(F)F)c(NC4CC4)n3)ccc(F)c21 ; CNc1nc(Nc2ccc3c(cnn3C)c2)ncc1C(F)(F)F ; CCNc1nc(Nc2cccc3c2cnn3CC(C)(C)O)nc2[nH]cc(Cl)c12 ; CNc1nc(Nc2ccc3nn(C)cc3c2)ncc1C(F)(F)F ; CS(=O)(=O)CCn1ncc2c(Nc3ncc(C(F)F)c(NC4CC4)n3)ccc(F)c21]

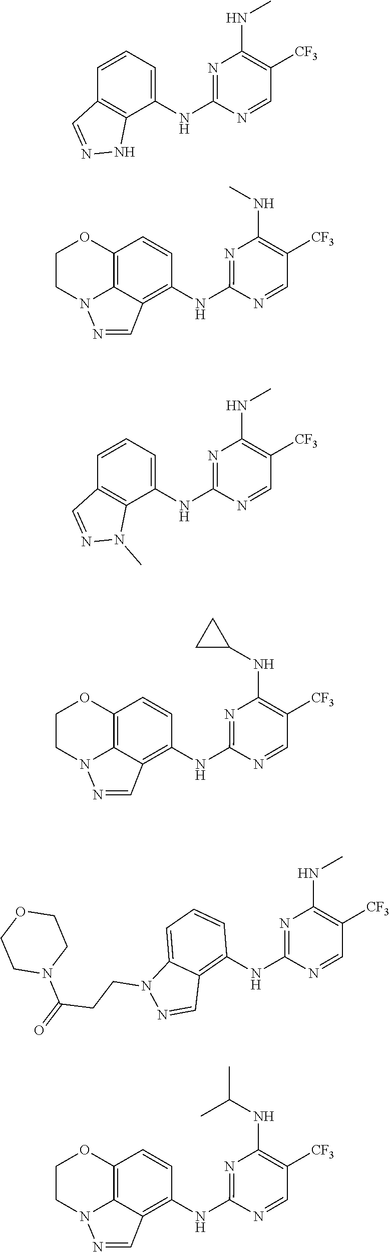 [SMILES: CNc1nc(Nc2cccc3cn[nH]c23)ncc1C(F)(F)F ; CNc1nc(Nc2ccc3c4c2cnn4CCO3)ncc1C(F)(F)F ; CNc1nc(Nc2cccc3cnn(C)c23)ncc1C(F)(F)F ; FC(F)(F)c1cnc(Nc2ccc3c4c2cnn4CCO3)nc1NC1CC1 ; CNc1nc(Nc2cccc3c2cnn3CCC(=O)N2CCOCC2)ncc1C(F)(F)F ; CC(C)Nc1nc(Nc2ccc3c4c2cnn4CCO3)ncc1C(F)(F)F]

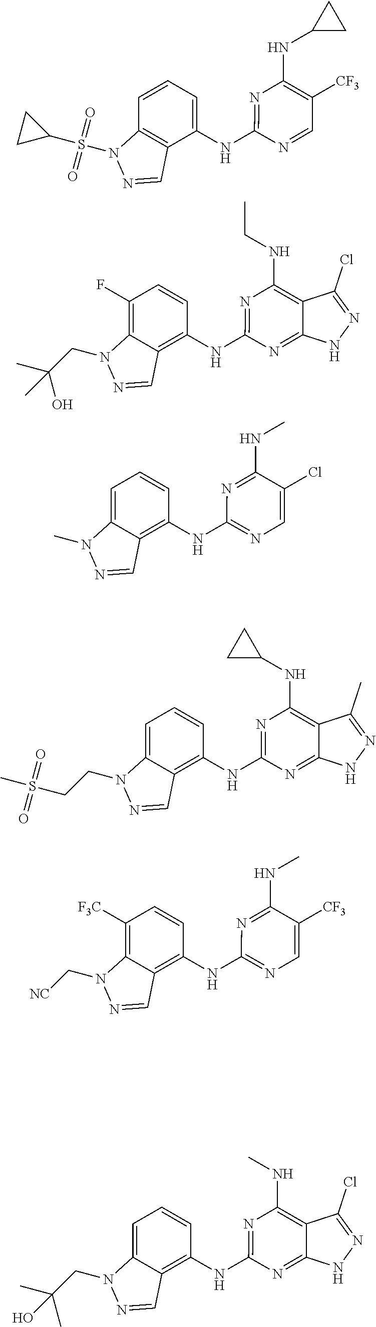 [SMILES: O=S(=O)(C1CC1)n1ncc2c(Nc3ncc(C(F)(F)F)c(NC4CC4)n3)cccc21 ; CCNc1nc(Nc2ccc(F)c3c2cnn3CC(C)(C)O)nc2[nH]nc(Cl)c12 ; CNc1nc(Nc2cccc3c2cnn3C)ncc1Cl ; Cc1n[nH]c2nc(Nc3cccc4c3cnn4CCS(C)(=O)=O)nc(NC3CC3)c12 ; CNc1nc(Nc2ccc(C(F)(F)F)c3c2cnn3CC#N)ncc1C(F)(F)F ; CNc1nc(Nc2cccc3c2cnn3CC(C)(C)O)nc2[nH]nc(Cl)c12]

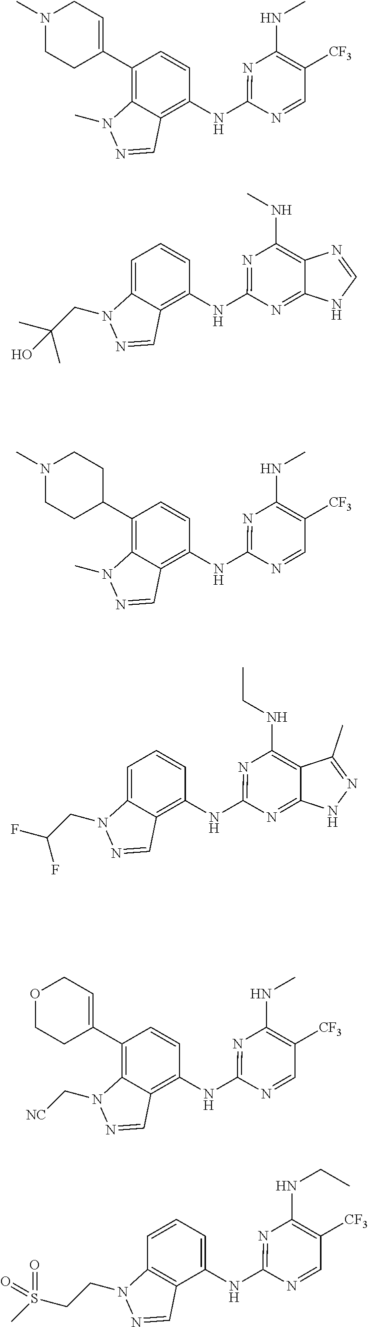 [SMILES: CNc1nc(Nc2ccc(C3=CCN(C)CC3)c3c2cnn3C)ncc1C(F)(F)F ; CNc1nc(Nc2cccc3c2cnn3CC(C)(C)O)nc2[nH]cnc12 ; CNc1nc(Nc2ccc(C3CCN(C)CC3)c3c2cnn3C)ncc1C(F)(F)F ; CCNc1nc(Nc2cccc3c2cnn3CC(F)F)nc2[nH]nc(C)c12 ; CNc1nc(Nc2ccc(C3=CCOCC3)c3c2cnn3CC#N)ncc1C(F)(F)F ; CCNc1nc(Nc2cccc3c2cnn3CCS(C)(=O)=O)ncc1C(F)(F)F]

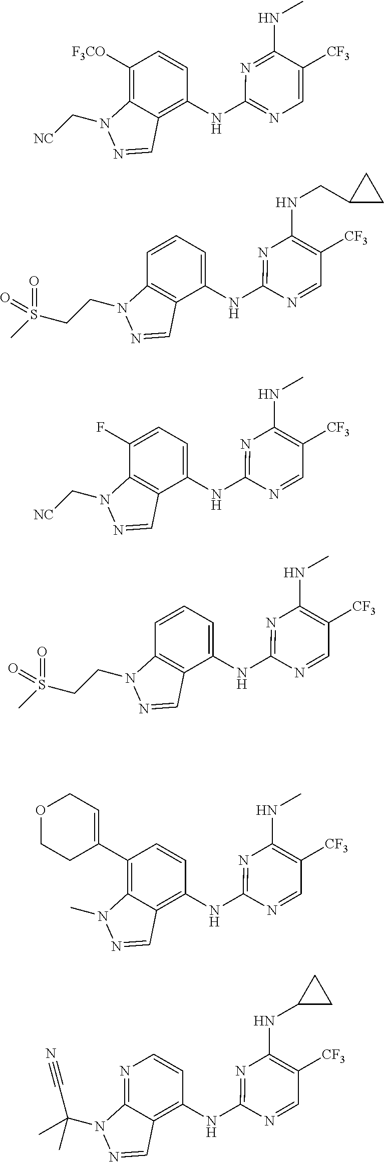 [SMILES: CNc1nc(Nc2ccc(OC(F)(F)F)c3c2cnn3CC#N)ncc1C(F)(F)F ; CS(=O)(=O)CCn1ncc2c(Nc3ncc(C(F)(F)F)c(NCC4CC4)n3)cccc21 ; CNc1nc(Nc2ccc(F)c3c2cnn3CC#N)ncc1C(F)(F)F ; CNc1nc(Nc2cccc3c2cnn3CCS(C)(=O)=O)ncc1C(F)(F)F ; CNc1nc(Nc2ccc(C3=CCOCC3)c3c2cnn3C)ncc1C(F)(F)F ; CC(C)(C#N)n1ncc2c(Nc3ncc(C(F)(F)F)c(NC4CC4)n3)ccnc21]

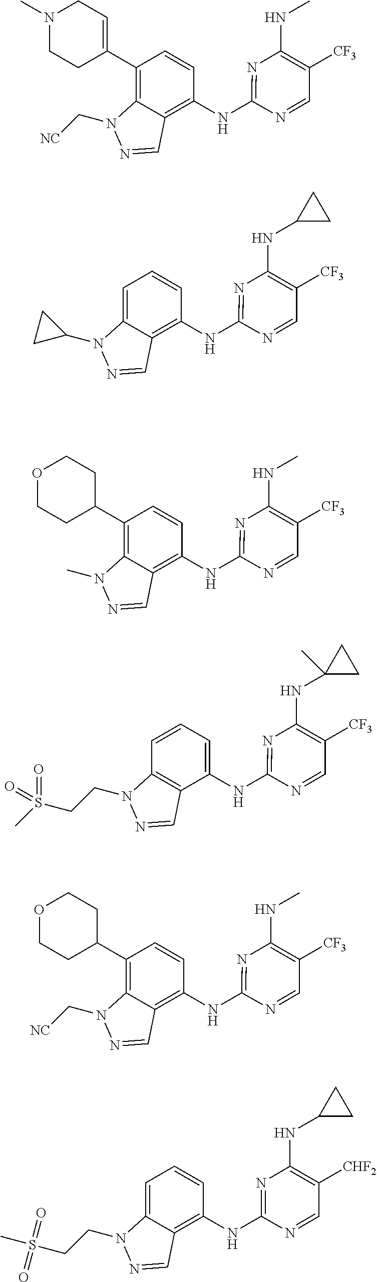 [SMILES: CNc1nc(Nc2ccc(C3=CCN(C)CC3)c3c2cnn3CC#N)ncc1C(F)(F)F ; FC(F)(F)c1cnc(Nc2cccc3c2cnn3C2CC2)nc1NC1CC1 ; CNc1nc(Nc2ccc(C3CCOCC3)c3c2cnn3C)ncc1C(F)(F)F ; CC1(Nc2nc(Nc3cccc4c3cnn4CCS(C)(=O)=O)ncc2C(F)(F)F)CC1 ; CNc1nc(Nc2ccc(C3CCOCC3)c3c2cnn3CC#N)ncc1C(F)(F)F ; CS(=O)(=O)CCn1ncc2c(Nc3ncc(C(F)F)c(NC4CC4)n3)cccc21]

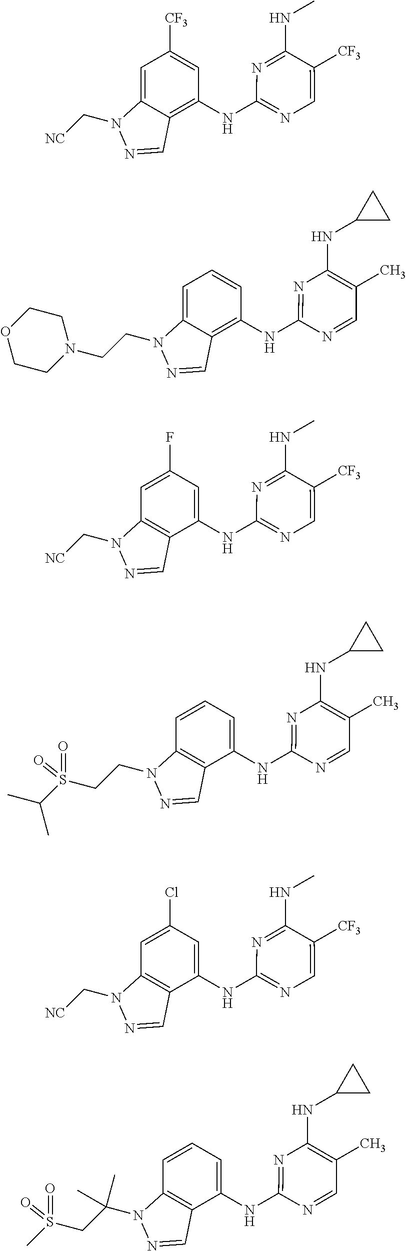 [SMILES: CNc1nc(Nc2cc(C(F)(F)F)cc3c2cnn3CC#N)ncc1C(F)(F)F ; Cc1cnc(Nc2cccc3c2cnn3CCN2CCOCC2)nc1NC1CC1 ; CNc1nc(Nc2cc(F)cc3c2cnn3CC#N)ncc1C(F)(F)F ; Cc1cnc(Nc2cccc3c2cnn3CCS(=O)(=O)C(C)C)nc1NC1CC1 ; CNc1nc(Nc2cc(Cl)cc3c2cnn3CC#N)ncc1C(F)(F)F ; Cc1cnc(Nc2cccc3c2cnn3C(C)(C)CS(C)(=O)=O)nc1NC1CC1]

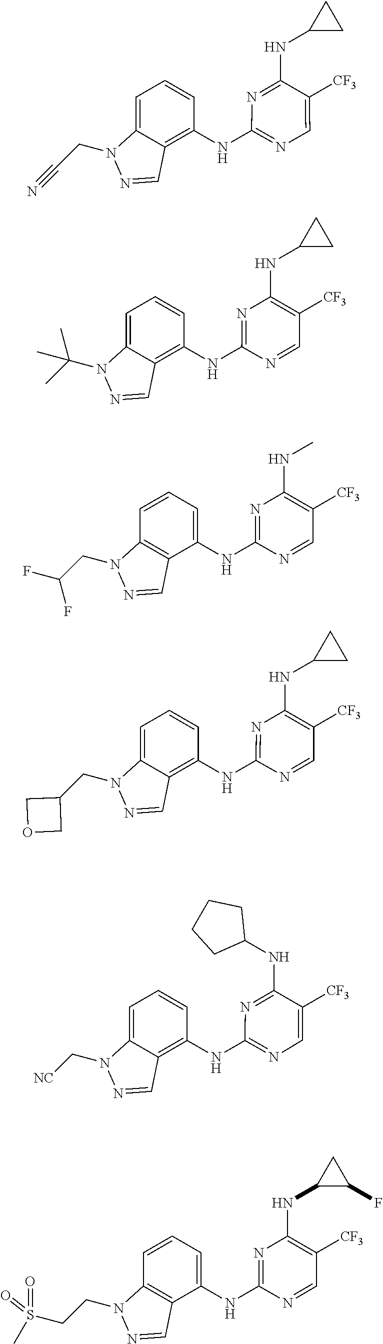

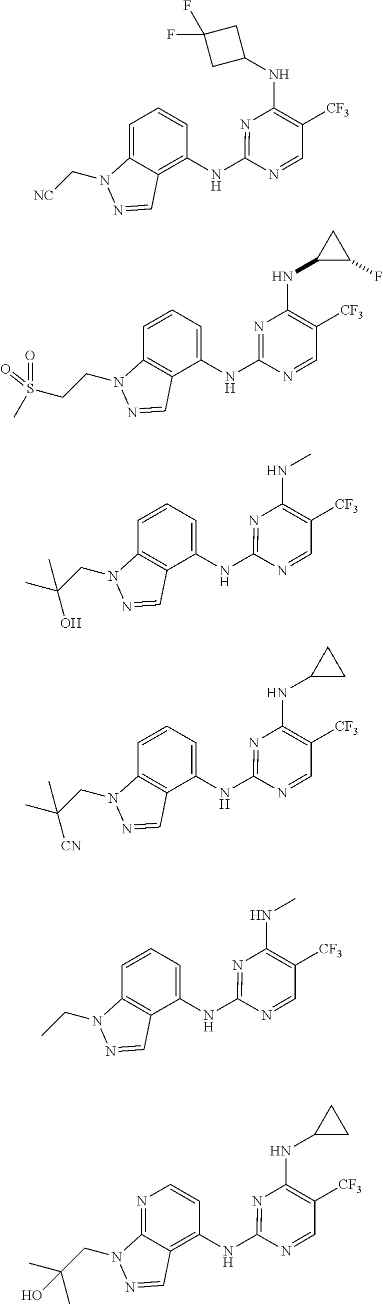

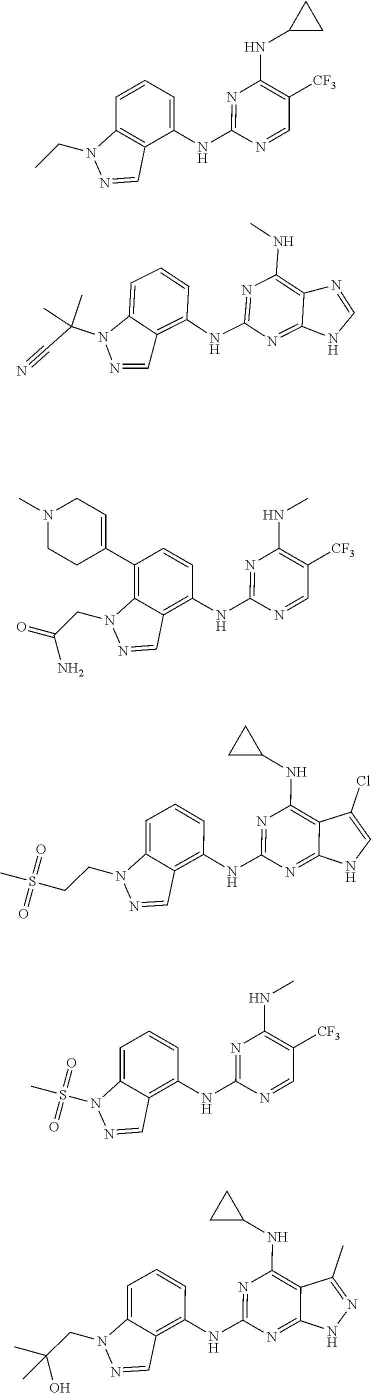 [SMILES: CCn1ncc2c(Nc3ncc(C(F)(F)F)c(NC4CC4)n3)cccc21 ; CNc1nc(Nc2cccc3c2cnn3C(C)(C)C#N)nc2[nH]cnc12 ; CNc1nc(Nc2ccc(C3=CCN(C)CC3)c3c2cnn3CC(N)=O)ncc1C(F)(F)F ; CS(=O)(=O)CCn1ncc2c(Nc3nc(NC4CC4)c4c(Cl)c[nH]c4n3)cccc21 ; CNc1nc(Nc2cccc3c2cnn3S(C)(=O)=O)ncc1C(F)(F)F ; Cc1n[nH]c2nc(Nc3cccc4c3cnn4CC(C)(C)O)nc(NC3CC3)c12]

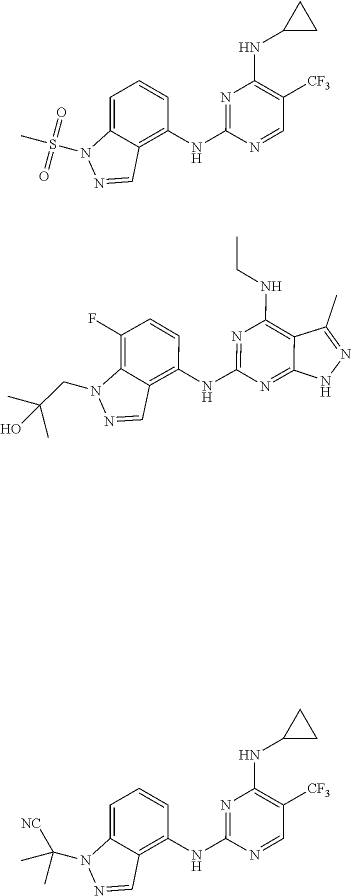 [SMILES: CS(=O)(=O)n1ncc2c(Nc3ncc(C(F)(F)F)c(NC4CC4)n3)cccc21 ; CCNc1nc(Nc2ccc(F)c3c2cnn3CC(C)(C)O)nc2[nH]nc(C)c12 ; CC(C)(C#N)n1ncc2c(Nc3ncc(C(F)(F)F)c(NC4CC4)n3)cccc21]

In one embodiment, the invention provides a pharmaceutical composition comprising a compound of the above Formulas (I)-(IV) and pharmaceutically acceptable salts thereof together with a pharmaceutically acceptable excipient.

In one embodiment, the invention provides compounds of the above Formulas (I)-(IV) and pharmaceutically acceptable salts thereof for use in therapy.

In one embodiment, the invention provides compounds of the above Formulas (I)-(V) and pharmaceutically acceptable salts thereof for use in a method for the treatment of a disease associated with LRRK2. The disease may be a neurological condition.

In one embodiment, the invention relates to the use of a compound of the above Formulas (I)-(IV) and pharmaceutically acceptable salts thereof in the manufacture of a medicament for use in the treatment of a disease associated with LRRK2.

In one embodiment, the invention relates to a method for the treatment of a disease associated with LRRK2, including PD, the method comprising the administration of a therapeutically effective amount of a compound of the above Formulas (I)-(IV) and pharmaceutically acceptable salts thereof to a patient in need thereof.

In another aspect, the invention provides methods of modulating the activity of a kinase by contacting cells containing a kinase with one or more compounds of the invention, such as any of those described above. The compound may inhibit activity of the kinase. The compound may increase activity of the kinase. The kinase may be LRRK2.

In embodiments of the use, the condition treated by the compounds of the invention is an autoimmune disease, inflammatory disease, bone disease, metabolic disease, neurological or neurodegenerative disease, cancer, cardiovascular disease, allergies, asthma, Alzheimer's disease, Parkinson's disease, skin disorder, eye disease, infectious disease, or hormone-related disease.

### DETAILED DESCRIPTION

### Chemical Definitions

The expression alkyl refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH—), iPr (—CH(CH₃)₂), nPr (—CH₂CH₂CH₃), n-Bu (—CH₂CH₂CH₂CH₃), or i-Bu (—CH₂CH(CH₃)₂).

The expression heteroalkyl refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₁₀ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”).

The expression alkenyl refers to a radical of a straight-chain or branched hydrocarbon group having from 2 to 20 carbon atoms, one or more carbon-carbon double bonds (e.g., 1, 2, 3, or 4 carbon-carbon double bonds) (“C₂₋₂₀ alkenyl”). In some embodiments, an alkenyl group has 2 to 10 carbon atoms (“²⁻¹⁰ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and 1 or 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroCC₂₋₆ alkenyl”).

The expression cycloalkyl refers to a saturated or partially unsaturated (for example, a cycloalkenyl group) cyclic group that contains one or more rings, e.g., 2 or 3 rings, and contains from 3 to 14 ring carbon atoms, such as from 3 to 10 (e.g., 3, 4, 5, 6 or 7) ring carbon atoms. The expression cycloalkyl refers furthermore to groups in which one or more hydrogen atoms have been replaced by fluorine, chlorine, bromine or iodine atoms or by OH, ═O, SH, ═S, NH₂, ═NH, N₃ or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetraline, cyclopentylcyclohexyl, fluorocyclohexyl or cyclohex-2-enyl group.

The expression cycloheteroalkyl or heterocycle refers to a cycloalkyl group as defined above in which one or more (e.g., 1, 2, or 3) ring carbon atoms have been replaced by an oxygen, nitrogen, silicon, selenium, phosphorus or sulfur atom or a SO group or a SO₂ group. A cycloheteroalkyl or heterocycle group may have 1 or 2 rings containing from 3 to 10 (e.g., 3, 4, 5, 6 or 7) ring atoms (e.g., C, O, N or S). Cycloheteroalkyl or heterocycle groups include cycloheteroalkenyl or heterocycloalkenyl groups. The expression cycloheteroalkyl or heterocycle refers furthermore to groups that are substituted by fluorine, chlorine, bromine or iodine atoms or by OH, ═O, SH, ═S, NH₂, ═NH, N₃ or NO₂ groups. Examples are a piperidinyl, prolinyl, imidazolidinyl, piperazinyl, morpholinyl, urotro pinyl, pyrrolidinyl, tetrahydrothiophenyl, tetrahydropyranyl, tetrahydrofuryl or 2-pyrazolinyl group and also lactams, lactones, cyclic imides and cyclic anhydrides.

The expression alkylcycloalkyl refers to groups that contain both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two rings having from 3 to 10 (e.g., 3, 4, 5, 6 or 7) ring carbon atoms, and one or two alkyl or alkynyl groups having 1 or 2 to 6 carbon atoms.

The expression heteroalkylcycloalkyl refers to alkylcycloalkyl groups as defined above in which one or more (e.g., 1, 2 or 3) carbon atoms have been replaced by an oxygen, nitrogen, silicon, selenium, phosphorus or sulfur atom or a SO group or a SO₂ group. A heteroalkylcycloalkyl group preferably contains 1 or 2 rings having from 3 to 10 (e.g., 3, 4, 5, 6 or 7) ring atoms, and one or two alkyl, alkenyl, alkynyl or heteroalkyl groups having from 1 or 2 to 6 carbon atoms. Examples of such groups are alkylheterocycle, alkylheterocycloalkenyl, alkenylheterocycle, alkynylheterocycle, heteroalkylcycloalkyl, heteroalkylheterocycle and heteroalkylheterocycloalkenyl, the cyclic groups being saturated or mono-, di- or tri-unsaturated.

The expression aryl refers to an aromatic group that contains one or more rings, e.g., 2 or 3 rings, containing from 6 to 14 ring carbon atoms, such as from 6 to 10 ring carbon atoms. The expression aryl refers furthermore to groups that are substituted by fluorine, chlorine, bromine or iodine atoms or by CH₃, OH, SH, NH₂, N₃ or NO₂ groups. Examples are the phenyl, naphthyl, biphenyl, 2-fluorophenyl, anilinyl, 3-nitrophenyl or 4-hydroxyphenyl group.

The expression heteroaryl refers to an aromatic group that contains one or more rings, e.g., 2 or 3 rings, containing from 5 to 14 ring atoms, such as from 5 to 10 ring atoms, and contains one or more (e.g., 1, 2, 3 or 4) oxygen, nitrogen, phosphorus or sulfur ring atoms. The expression heteroaryl refers furthermore to groups that are substituted by fluorine, chlorine, bromine or iodine atoms or by CH₃, OH, SH, N₃, NH₂ or NO₂ groups. Examples are pyridyl (e.g. 4-pyridyl), imidazolyl (e.g. 2-imidazolyl), phenylpyrrolyl (e.g. 3-phenylpyrrolyl), thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl, pyridazinyl, oxazolyl, isoxazolyl, triazolyl, tetrazolyl, isoxazolyl, indazolyl, indolyl, benzimidazolyl, benzoxazolyl, benzisoxazolyl, benzthiazolyl, pyridazinyl, quinolinyl, isoquinolinyl, pyrrolyl, purinyl, carbazolyl, acridinyl, pyrimidyl, 2,3′-bifuryl, pyrazolyl (e.g. 3-pyrazolyl) and isoquinolinyl groups.

The expression aralkyl refers to groups containing both aryl and also alkyl, alkenyl, alkynyl and/or cycloalkyl groups in accordance with the above definitions, such as, for example, aryl-alkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetraline, dihydronaphthalene, indanone, phenylcyclopentyl, cumene, cyclohexylphenyl, fluorene and indane. An aralkyl group preferably contains one or two aromatic ring systems containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (e.g., 1, 2, 3 or 4) carbon atoms have been replaced by an oxygen, nitrogen, silicon, selenium, phosphorus, boron or sulfur atom, that is to say to groups containing both aryl or heteroaryl, respectively, and also alkyl, alkenyl, alkynyl and/or heteroalkyl and/or cycloalkyl and/or heterocycle groups in accordance with the above definitions. A heteroaralkyl group preferably contains one or two aromatic ring systems containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, wherein 1, 2, 3 or 4 of these carbon atoms have been replaced by oxygen, sulfur or nitrogen atoms.

Examples are arylheteroalkyl, arylheterocycle, arylheterocycloalkenyl, arylalkyl heterocycle, arylalkenylheterocycle, arylalkynylheterocycle, arylalkylhetero cycloalkenyl, heteroarylalkyl, heteroarylalkenyl, heteroarylalkynyl, heteroarylheteroalkyl, heteroarylcycloalkyl, heteroarylcycloalkenyl, heteroarylheterocycle, hetero arylheterocycloalkenyl, heteroarylalkylcycloalkyl, heteroarylalkylheterocycloalkenyl, hetero aryiheteroalkylcycloalkyl, heteroarylheteroalkylcycloalkenyl and heteroarylheteroalkylhetero cycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

As stated above, the expressions cycloalkyl, cycloheteroalkyl, heterocycle, alkylcycloalkyl, heteroalkylcycloalkyl, aryl, heteroaryl, aralkyl and heteroaralkyl also refer to groups that are substituted by fluorine, chlorine, bromine or iodine atoms or by CH₃, OH, ═O, SH, ═S, NH₂, ═NH, N₃ or NO₂ groups.

The expression carbocyclyl or carbocyclic refers to a radical of a non-aromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms 10 (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C), cyclohexenyl (C₆), cyclohexadienyl (C₆), and the like.

Exemplary C₃₋ₛ carbocyclyl groups include, without limitation, the aforementioned C₃-carbocyclyl groups as well as cycloheptyl (C₇), cycloheptenyl (C₇), cycloheptadienyl (C₇), cycloheptatrienyl (C₇), cyclooctyl (G), cyclooctenyl (G), bicyclo[2.2.1]heptanyl (C₇), bicyclo[2.2.2]octanyl (G), and the like. Exemplary C₃₋₁₀ carbocyclyl groups include, without 20 limitation, the aforementioned G-s carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

The expression heterocyclyl or heterocyclic refers to a radical of a 3- to 14-membered non-aromatic ring system having ring carbon atoms and 1 to 4 ring heteroatoms, wherein each heteroatom is independently selected from nitrogen, oxygen, sulfur, boron, phosphorus, and silicon (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1¬4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups 5 containing two heteroatoms include, without limitation, piperazinyl, morpholinyl, dithianyl, dioxanyl. Exemplary 6-membered heterocyclyl groups containing two heteroatoms include, without limitation, triazinanyl, Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like.

Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The expression optionally substituted means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Heteroatoms, such as nitrogen, may have substituents, such as any suitable substituent described herein which satisfies the valencies of the heteroatoms and results in the formation of a stable moiety.

For example, and without limitation, optional substituents include fluorine, chlorine, bromine, and iodine atoms and CF₃, CN, OH, ═O, SH, ═S, NH₂, ═NH, N₃ and NO₂ groups. Optional substituents also include C₁-C₁₀ alkyl, C₂-C₁₀ alkenyl, C₁-C₁₀ heteroalkyl, C₃-C₁₆ cycloalkyl, C₂-C₁₇ heterocycle, C₄-C₂₀ alkylcycloalkyl, C₂-C₁₉ heteroalkylcycloalkyl, C₆-C₁₈ aryl, C₁₋₁₇ heteroaryl, C₇-C₂₀ aralkyl or C₂-C₁₉ heteroaralkyl, C₁-C₆ alkyl, C₂-C₆ alkenyl, C₁-C₆ heteroalkyl, C₃-C₁₀ cycloalkyl, C₂-C₉ heterocycle, C₇-C₁₂ alkylcycloalkyl, C₂-C₁₁ heteroalkylcycloalkyl, C₆-C₁₀, aryl, C₁-C₉ heteroaryl, C₇-C₁₂ aralkyl, C₂-C₁₁ heteroaralkyl, and C₁-C₁₀ haloalkyl groups.

Exemplary substituents are F, Cl, Br, OH, SH, ═O, NH₂, amino, C₁₋₄ alkyl, C₁₋₄ heteroalkyl cyclopropyl, SF₅, NO, NO₂.

Other exemplary substituents are F, Cl, Br, OH, SH, ═O, NH₂, C₁₋₄ alkyl (e.g. methyl, ethyl, t-butyl), NMe₂, CONH₂, CH₂NMe₂, NHSO₂Me, C(CH₃)2CN, COMe, OMe, SMe, COOMe, COOEt, CH₂COOH, OCH₂COOH, COOH, SOMe, SO₂Me, cyclopropyl, SO₂NH₂, SO₂NHMe, SO₂CH₂CH₂OH, NHCH₂CH₂OH, CH₂CH₂OCH₃, SF, SO₂NMe₂, NO, NO₂, OCF₃, SO₂CF₃, CN or CF₃.

Other exemplary substituents are F, Cl, Br, Me, OMe, CN or CF₃.

The term halogen preferably refers to F, Cl, Br or I.

When an aryl, heteroaryl, cycloalkyl, alkylcycloalkyl, heteroalkylcycloalkyl, heterocycle, aralkyl or heteroaralkyl group contains more than one ring, these rings may be bonded to each other via a single or double bond or these rings may be annulated.

According to certain embodiments, all alkyl, alkenyl, alkynyl, heteroalkyl, aryl, heteroaryl, cycloalkyl, heterocycle, alkylcycloalkyl, heteroalkylcycloalkyl, aralkyl and heteroaralkyl groups described herein may optionally be substituted.

Other optional substituents include, but are not limited to, halogen, —CN, —NO₂, —N, SO₂H, —SO₃H, —OH, —OR^{aa}, —ON(R^{bb})₂, —N(R^{bb})₂, —N(R^{bb})₃⁺X, —N(OR^{c})R^{bb}, —SH, —SR^{aa}, —SSR^{CC}, —C(O)R^{aa}, —CO₂H, —CHO, —C(OR^{cc})₂, —CO₂R^{aa}, —OC(O)R^{aa}, —OCO₂R^{aa}, —C(O)N(R_{bb})₂, —C(O)N(R^{aa})(R^{bb}), —OC(O)N(R^{bb})₂, —NR^{bb}C(O)R^{aa}, —NR^{bb}CO₂^{aa}, —NR^{bb}C(O)N(R^{bb})₂, —C(NR^{bb})R^{aa}, —C(NR^{bb})OR^{aa}, —OC(NR^{bb})R^{aa}, —OC(NR^{bb})OR^{aa}, —C(NR^{bb})N(R^{bb})₂, —OC(NR^{bb})N(R^{bb})₂, —NR^{bb}C(NR^{bb})N(R^{bb})₂, —C(O)NR^{bb}SO₂R^{aa}, —NR^{bb}SO₂R^{aa}, —SO₂N(R^{bb})₂, —SO₂R^{aa}, SO₂OR^{aa}, —OSO₂R^{aa}, —S(O)R^{aa}, e.g., —S(O)R^{aa}, —OS(O)R^{aa}, —Si(R^{aa})₃, —OSi(R^{aa})₃—C(S)N(R^{bb})₂, —C(O)SR^{aa}, —C(S)SR^{aa}, —SC(S)SR^{aa}, —SC(O)SR^{aa}, —OC(O)SR^{aa}, —SC(O)OR^{aa}, —SC(O)R^{aa}, —P(O)₂R^{aa}, —OP(O)₂R^{aa}, —P(O)(R^{aa})₂, —OP(O)(R^{aa})₂, —OP(O)(OR^{cc})₂, —P(O)₂N(R^{bb})₂, —OP(O)₂N(R^{bb})₂, —P(O)(NR^{bb})₂, —OP(O)(NR^{bb})₂, —NR^{bb}P(O)(OR^{cc})₂, —NR^{bb}P(O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, wherein each alkyl, alkenyl, carbocyclyl, heterocyclyl, aryl, and heteroaryl is independently substituted with 0, 1, 2, 3, 4, or 5 R_{dd} groups; or two geminal hydrogens on a carbon atom are replaced with the group ═O, ═S, ═NN(R^{bb})₂, —NR^{bb}C(O)R^{aa}, ═NNR^{bb}C(O)OR^{aa}, ═NNR^{bb}S(O)₂Ra, ═NR^{bb}, or ═NOR^{cc}; in which:
each instance of R^{aa} is, independently, selected from C₁₋₁₀ alkyl, C₁₋₁₀ heteroalkyl, C₁₋₁₀ haloalkyl, (C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered cycloalkyl, 3-14 membered cycloheteroalkyl, 3-14 membered heterocyclyl or 5-14 membered heteroaryl ring, wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(O)R^{aa}, —C(O)N(R^{cc})₂, CO₂R^{aa}, —SO₂R^{aa}, —C(NR^{cc})OR^{aa}, —C(NR^{cc})N(R^{cc})₂, —SO₂N(R^{cc})₂, —SO₂R^{cc}, —SO₂OR^{cc}, —SOR^{aa}, —C(S)N(R^{cc})₂, —C(O)SR^{cc}, —C(S)SR^{cc}, —P(O)₂R^{aa}, —P(O)(R^{aa}), —P(O)₂N(R^{cc})₂, —P(O)(NR^{cc})₂, C₁₋₁₀ alkyl, C₁₋₁₀ heteroalkyl, C₁₋₁₀ haloalkyl, C₂₋₁₀ alkenyl, C₃₋₁₀ cycloalkyl, C₃₋₁₀ cycloheteroalkyl, C₃₋₁₀ cycloalkenyl, C₃₋₁₀ cycloheteroalkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, heteroalkyl, alkenyl, cycloalkyl, cycloheteroalkyl, cycloalkenyl, cycloheteroalkenyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ haloalkyl, C₂₋₁₀ alkenyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(Rn); CX˜, —N(OR^{ee})R^{ff}, —SH, —SR^{ee}, —SSR^{ee}, —C(O)R^{ee}, —CO₂H, —CO₂R^{ee}, —OC(O)R^{ee}, —OCO₂R^{ee}, —C(O)N(R)₂, —OC(O)N(R^{ff})₂, —NR^{ff}C(O)R^{ee}, —NR^{ff}CO₂R^{ee}, —NR^{ff}C(O)N(R^{ff})₂, —C(NR^{ff})OR^{ee}, —OC(NR^{ff})R^{ee}, —OC(NR^{ff})OR^{ee}, —C(NR^{ff})N(R^{ff})₂, —OC(NR^{ff})N(R^{ff})₂, —NR^{ff}C(NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(O)R^{ee}, e.g., —S(O)R^{ee}, —Si(R^{ee})₃, —OSi(R^{ee})₃, —C(S)N(R^{ff})₂, —C(O)SR^{ee}, —C(S)SR^{ee}, —SC(S)SR^{ee}, —P(O)₂R^{ee}, —P(O)(R^{ee})₂, —OP(O)(R^{ee})₂, —OP(O)(OR^{ee})₂, C₁₋₆ alkyl, C₁₋₆ heteroalkyl, C₁₋₆ haloalkyl, (C₂₋₆ alkenyl, C₃₋₁₀ carbocyclyl, 3-10 membered heterocyclyl, (C₆₋₁₀ aryl, 5-10 membered heteroaryl, wherein each alkyl, alkenyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, wherein each alkyl, alkenyl, carbocyclyl, heterocyclyl, aryl, and heteroaryl is independently substituted with 0, 1, 2, 3, 4, or 5 R^{gg} groups;
each instance of R^{ff} is, independently, selected from hydrogen C₁₋₆ alkyl, C₁₋₆ haloalkyl, C₂₋₆ alkenyl, C₃₋₁₀ carbocyclyl, 3-10 membered heterocyclyl, C₆₋₁₀ aryl, 5-10 membered heteroaryl, or two R^{ff} groups are joined to form a 3-14 membered heterocyclyl or 5-14 membered heteroaryl ring, wherein each alkyl, alkenyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N((C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻-MR⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(O)(C₁₋₆ alkyl), —CO₂H, —CO₂(C₁₋₆ alkyl), —OC(O)(C₁₋₆ alkyl), —OCO₂(C₁₋₆ alkyl), —C(O)NH₂, —C(O)N(C₁₋₆ alkyl)₂, —OC(O)NH(C₁₋₆ alkyl), —NHC(O)(C₁₋₆ alkyl), —N(C₁₋₆ alkyl)C(O)(C₁₋₆ alkyl), —NHCO(C₁₋₆ alkyl), —NHC(O)N(C₁₋₆ alkyl)₂, —NHC(O)NH(C₁₋₆ alkyl), —NHC(O)NH₂, —C(NH)O(C₁₋₆ alkyl), —OC(NH)(C₁₋₆ alkyl), —OC(NH)OC₁₋₆ alkyl, —C(NH)N(C₁₋₆ alkyl)₂, —C(NH)NH(C₁₋₆ alkyl), —C(NH)NH₂, —OC(NH)N(C₁₋₆ alkyl)₂, —OC(NH)NH(C₁₋₆ alkyl), —OC(NH)NH₂, —NHC(NH)N(C₁₋₆ alkyl)₂, —NHC(NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl), —OSi(C₁₋₆ alkyl)₃, —C(S)N(C₁₋₆ alkyl)₂, C(S)NH(C₁₋₆ alkyl), C(S)NH₂, —C(O)S(C₁₋₆ alkyl), —C(S)SC₁₋₆ alkyl, —SC(S)SC₁₋₆ alkyl, —P(O)₂(C₁₋₆ alkyl), —P(O)(C₁₋₆ alkyl)₂, —OP(O)(C₁₋₆ alkyl)₂, —OP(O)(OC₁₋₆ alkyl)₂, C₁₋₆ alkyl, C₁₋₆ haloalkyl, C₂₋₆ alkenyl, C₃₋₁₀ carbocyclyl, C₃₋₁₀ aryl, 3-10 membered heterocyclyl, 5-10 membered heteroaryl; or two geminal R^{gg} substituents can be joined to form ═O or ═S; wherein X⁻ is a counterion.

### Compounds

In an aspect, the invention provides compounds of formula (I):

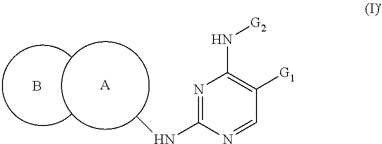

and pharmaceutically acceptable salts thereof,
wherein:
G₁ is CF₃, CHF₂, CH₂F, halogen or cyclopropyl, ethyl, isopropyl;
G₂ is H, substituted or unsubstituted C₁-C₆-alkyl, C₃-C₆-cycloalkyl, C₁-C₆ haloalkyl, C₃-C₆-halocycloalkyl, 1-6 membered heterocycles, heteroaryl, —CH₂-cycloalkyl, —CF₂-cycloalky, —CH(CH₃)-cycloalkyl, —CH₂-aryl, —CH₂-heterocycle, —CH₂-heteroaryl, —CF₂-aryl, and —CH(—CH₃)-aryl;
A and B are independently selected from 5 or 6 membered cycloalkyl or cycloaryl rings comprising one or more heteroatoms and one or more substitutions;
wherein both A and B comprise, cumulatively, at least two (2) nitrogen heteroatoms;
wherein A and B are fused at 2 positions;
wherein the one or more substitutions on the A and B rings are independently selected from: H, halo, C₁-C₆-alkyl, branched alkyl, haloalkyl, substituted or unsubstituted alkenyl, substituted or unsubstituted alkynyl, alkoxy, cycloalkoxy, haloalkoxy, alkoxyalkoxy, cyano, —CH₂-cycloalkyl, —CF₂-cycloalky, —(CH₂)₀₋₂C(CH₃)₂—CN, —(CH₂)₀₋₄—CN, —(CH₂)₀₋₄SO₂R′, —CH(CH₃)-cycloalkyl, —CH₂-aryl, —CF₂-aryl, —CH(—CH₃)-aryl, —C(═O)-alkyl, —C(═O)cycloalkyl, —C(═O)—NR′R″, —C(═O)—NH-alkyl, —C(═O)NH₂, hydroxy, —COOH (and ester thereof), alkylsulfonyl, cycloalkylsulfonyl, arylsulfonyl, —S(═O)₂—NR′R″, —NH₂, —NR′R″, —NR′—S(═O)₂R″, cyanoalkyl, haloalkyl, substituted or unsubstituted alkylsulfonyl, arylsulfonyl, morpholine, —C(═O)-heterocycles, —C(H)₀₋₁(—CH₃)₁₋₂—OH, —CH₂—C(═O)—NH₂; 3-6 membered heterocycle, any of which may have one or more substituents, wherein the 3-6 membered heterocycle comprises at least one heteroatom independently selected from O, S, and N;
wherein R′ and R″ are independently selected from a group consisting of H, alkyl, substituted or unsubstituted aryl, and heterocycles.

In certain embodiments, the compound of the invention is a compound of Formula (II):

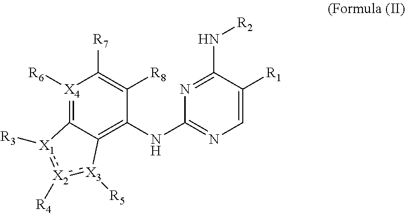

wherein,
X₁, X₂, X₃, and X₄ are C or N, wherein X₁, X₂, and X₃ include either one (1) N, two (2) N, or three (3) N;
R₁ is CF₃, CHF₂, CH₂F, halogen, cyclopropyl, ethyl, isopropyl;
R₂ is H, substituted or unsubstituted C₁-C₆ alkyl, C₃-C₆-cycloalkyl, C₃-C₆-halocycloalkyl; branched alkyl, alkenyl, alkynyl, haloalkyl, alkoxy, cycloalkoxy, haloalkoxy, —CH₂-cycloalkyl, —CF₂-cycloalky, tetrahydrofuran, —CH(—CH₃)-cycloalkyl, —CH₂-aryl, —CF₂-aryl, —CH(—CH₃)-aryl, —C(═O)-alkyl, —C(═O)cycloalkyl, —C(═O)—NH-alkyl;
R₃, R₄, R₅ are independently selected from a group consisting of: H, substituted or unsubstituted C₁-C₆ alkyl, C₃-C₆-cycloalkyl, C₃-C₆-halocycloalkyl; branched alkyl, alkenyl, alkynyl, haloalkyl, alkoxy, cycloalkoxy, haloalkoxy, nitro, cyano, alkylcyano, —CH₂-cycloalkyl, —CF₂-cycloalky, —CH(CH₃)-cycloalkyl, —CH₂-aryl, —CF₂-aryl, —CH(—CH₃)-aryl, —C(═O)—R′, —C(═O)—NR′R″, —S═(O)₂—R′, —CH₂CN, —C—(CH₃)₂—CN, —C(CH₃)₂—OH, —CH₂—C(═O)—NH₂;
R₆, R₇, and R₈ are independently selected from a group consisting of: H, halo, C₁-C₆-alkyl, branched alkyl, alkenyl, alkynyl, haloalkyl, alkoxy, cycloalkoxy, haloalkoxy, nitro, cyano, —CH₂-cycloalkyl, —CF₂-cycloalky, —CH(CH₃)-cycloalkyl, —CH₂-aryl, —CF₂-aryl, —CH(—CH₃)-aryl, —C(═O)-alkyl, —C(═O)cycloalkyl, —C(═O)—NH-alkyl, —C(═O)NH₂, hydroxy, —COOH (and ester thereof), —C(═O)-morpholine, —C(═O)-heterocycle, —S═(O)₂—R, amino, —NR′R″, —NR′S═(O)₂—R′, cyanoalkyl, haloalkyl, —C(—CH₃)₂—OH, —CH₂—C(═O)—NH₂; 3-6 membered heterocycle, any of which may have one or more substituents, wherein the 3-6 membered heterocycle comprises at least one heteroatom independently selected from O, S, and N; —S═(O)₂-cyclopropyl, —C(═O)-morpholine, —C(—CH₃)₂—OH, —CH₂—C(═O)—NH₂, —CF₃, —OCF₃, tetrahydropyran, 3H-pyran, 2H-pyran, piperidine, alkyl-morpholine;
wherein R′ and R″ are independently selected from a group consisting of H, unsubstituted or substituted C₁-C₈ alkyl, substituted or unsubstituted C₁-C₈ aryl, substituted or unsubstituted C₁-C₈ cycloalkyl or heterocycloalkyl;
wherein substitutions R₃, R₄, R₅, and R₆ are only present where valency permits; and
wherein R₃ and R₆ optionally form a 5-7 membered heteroalkyl ring.

In certain embodiments, R₁ is CF₃.

In certain embodiments, R₁ is CHF₂.

In certain embodiments, wherein R₁ is chloro.

In certain embodiments, wherein R₂ is H, methyl, ethyl, cyclopropyl, halogentated cyclopropyl, ethyl, cyclopentyl, tetrahydrofuran, cyclobutyl, hydroxycyclobutyl, halocyclopropyl, fluorocycopropyl, diflurocyclobutyl, isopropyl, —CH₂CF₃, —CH₂—CH₂—F, —CH₂—CH₂—OH, —CH₂—CH₂—O—CH, —CH₂—CH₂S═(O)₂—CH₃, —CH₂-cyclopropyl, —N(CH₃)₂, —CH(CH₃)₂, cyclobutyl, or —CH(CH₃)cyclopropyl.

In certain embodiments, wherein R₃, R₄, R₅ are independently selected from a group consisting of: H, —CH₃, —CH₂—CN, —CH₂—CH₂—O—CH₃, —CH₂—CHF₂, isopropyl, cyanoisopropyl, cyclopropyl, t-butyl, —CH₂-cyclopropyl, —CH₂—C(CH3)₂—CN, —S═(O)₂—CH₃, —CH₂—CH₂S═(O)₂—CH₃, —C—(CH₃)₂—CN, —S═(O)₂—CH—(CH₃)₂, —S═(O)₂-cyclopropyl, —CH₂—CH₂—S═(O)₂—CH₂—CH₃, —C—(CH₃)₂—S═(O)₂—CH—(CH₃)₂, —S═(O)—CH₂—CH₂—OCH₃, -morpholine, —CH₂-oxetane, C(═O)-morpholine, —C(—CH₃)₂—OH, —CH₂—C(═O)—NH₂.

In certain embodiments, wherein R₆, R₇, and R₈ are independently selected from a group consisting of: H, fluoro, methyl, chloro, cyano, —O—CH₃, CH₃, —CH—(CH₃)₂, —CH₂—CN, —CH₂—CHF₂, —S═(O)₂—CH₃, —C—(CH₃)₂—CN, —S═(O)₂-cyclopropyl, —C(═O)-morpholine, —C(—CH₃)₂—OH, —CH₂—C(═O)—NH₂, —CF₃, —OCF₃, tetrahydropyran, 3H-pyran, 2H-pyran, piperidine, methylpiperidine, alkyl-morpholine.

In certain the embodiments, R1 and R2 together form a ring.

In certain embodiments, R₃ and R₆ together form a fused ring.

In certain embodiments, R₃ and R together form a morpholine ring

In certain embodiments, X₁ and X₂ are N, and X₃ is C.

In another aspect, the compound of the invention is a compound of Formula (III):

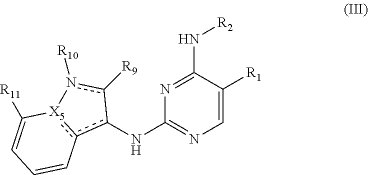

wherein, X₅ is selected from C or N;
R₉, R₁₀, or R₁₁ are independently selected from a group consisting of: H, substituted or unsubstituted C₁-C₆ alkyl, C₁-C₆-cycloalkyl, C₃-C₆-halocycloalkyl; C₁-C₆-alkyl, branched alkyl, alkenyl, alkynyl, haloalkyl, alkoxy, cycloalkoxy, haloalkoxy, nitro, cyano, —CH₂-cycloalkyl, —CF₂-cycloalky, —CH(CH₃)-cycloalkyl, —CH₂-aryl, —CF₂-aryl, —CH(—CH₃)-aryl, C(═O)-alkyl, —C(═O)cycloalkyl, —C(═O))—NH-alkyl, —S═(O)₂—CH₃, —C—(CH₃)₂—CN, —S═(O)₂-cyclopropyl, —C(═O)-morpholine, —C(—CH₃)₂—OH, —CH₂—C(═O)—NH₂;
wherein substitutions R₉, R₁₀, and R₁₁ are only present where valency permits.

In certain embodiments, X₅ is C.

In certain embodiments, X₅ is N.

In certain embodiments, R₉, R₁₀, or R₁₁ are independently selected from a group consisting of: H, fluoro, chloro, cyano, CH, —O—CH₃, —CH—(CH₃)₂, or —CH₂—CN.

In another aspect, the compound is a compound of Formula (IV):

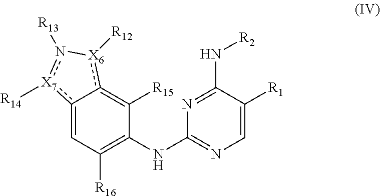

wherein, X₆ and X₇ are selected from C or N, wherein at least one of X₆ and X₇ is N;
R₁₂, R₁₃, and R₁₄ are independently selected from a group consisting of: H, substituted or unsubstituted C₁-C₆ alkyl, C₃-C₆-cycloalkyl, C₃-C₆-halocycloalkyl; C₁-C₆ branched alkyl, C₁-C₆-alkenyl, C₁-C₆-alkynyl, C₁-C₆-haloalkyl, C₁-C₆-alkoxy, cycloalkoxy, haloalkoxy, halo, nitro, cyano, —CH₂-cycloalkyl, —CF₂-cycloalky, —CH(CH₃)-cycloalkyl, —CH₂-aryl, —CF₂-aryl, —CH(—CH—)-aryl, C(═O)-alkyl, —C(═O)heterocycloalkyl, and —C(═O)-morpholine;
R₁₅ and R₁₆ are independently selected from a group consisting of: H, halo, substituted or unsubstituted C₁-C₆ alkyl, alkyloxy, cyanoalkyl, C₁-C₆-branched alkyl, alkenyl, alkynyl, haloalkyl, alkoxy, cycloalkoxy, haloalkoxy, halo, nitro, cyano, —CH₂-cycloalkyl, —C₂-cycloalky, —CH(CH₃)-cycloalkyl, —C(═O)heterocycloalkyl, and —C(═O)-morpholine;
wherein substitutions R₁₂, R₁₃, and R₁₄ are only present where valency permits.

In certain embodiments, X, in N and X₇ is C.

In certain embodiments, X₆ in C and X₇ is N.

In certain embodiments, R₁₂, R₁₃, and R₁₄ are independently selected from a group consisting of: H, fluoro, chloro, bromo, cyano, CH₃, —CH—(CH₃)₂, —CH₂—CN and —C(═O)-morpholine.

In certain embodiments, R₁₅ and R₁₆ are independently selected from a group consisting of: H, fluoro, chloro, bromo, CH₃, —O—CH₃, —O—CF₃, —CF₃, ethyl, vinyl, or —CH₂—CN.

In another aspect, the compound of the invention is a compound of Formula (V):

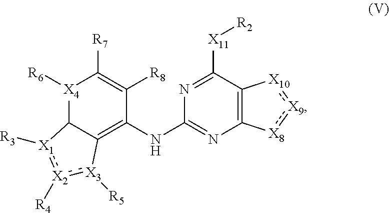

wherein X₈ is N or NH;
X is CH or N,
X₁₀ is NR₁₇, N, or CR₁₇;
X₁₁ is O or NH.
R₁₇ is selected from the group consisting of H, halo, and alkyl.

In certain embodiments, R₁₇ is chloro.

In certain embodiments, R₁₇ is methyl.

In certain embodiments, the compound of the invention is selected from:

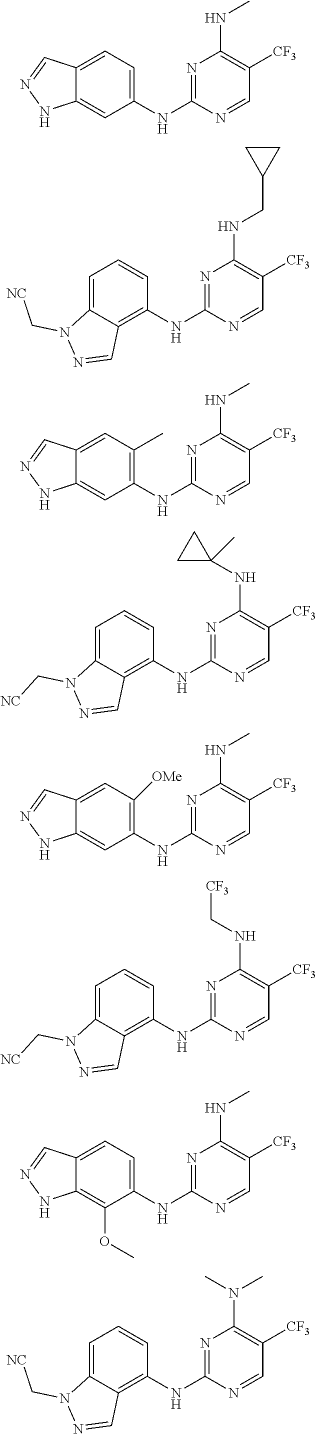

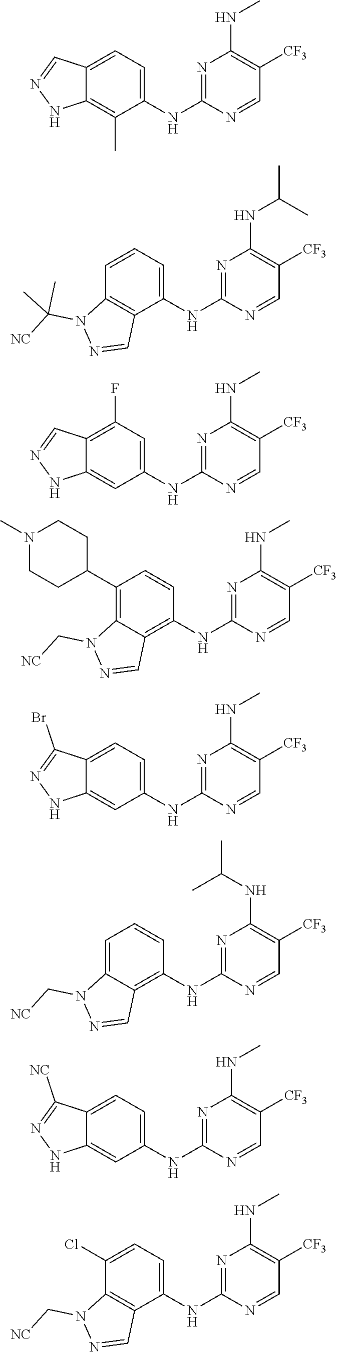

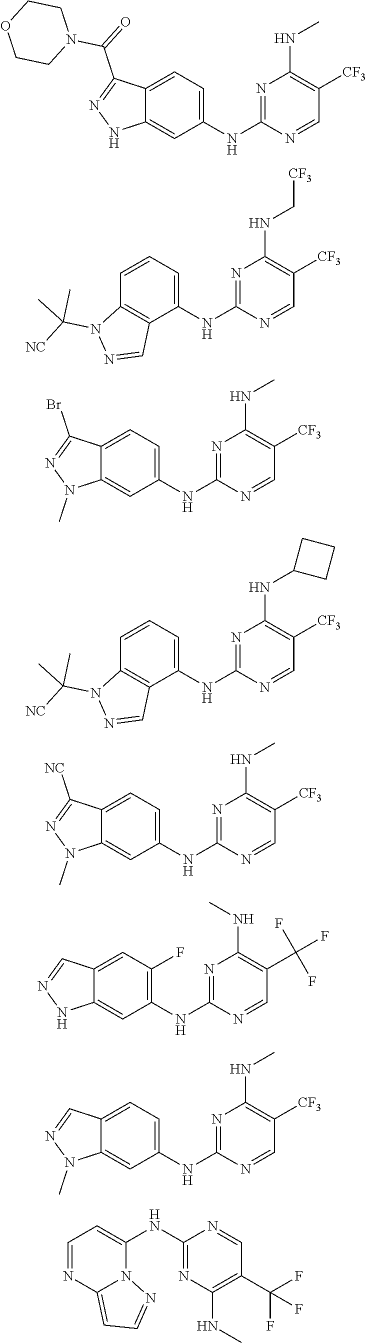

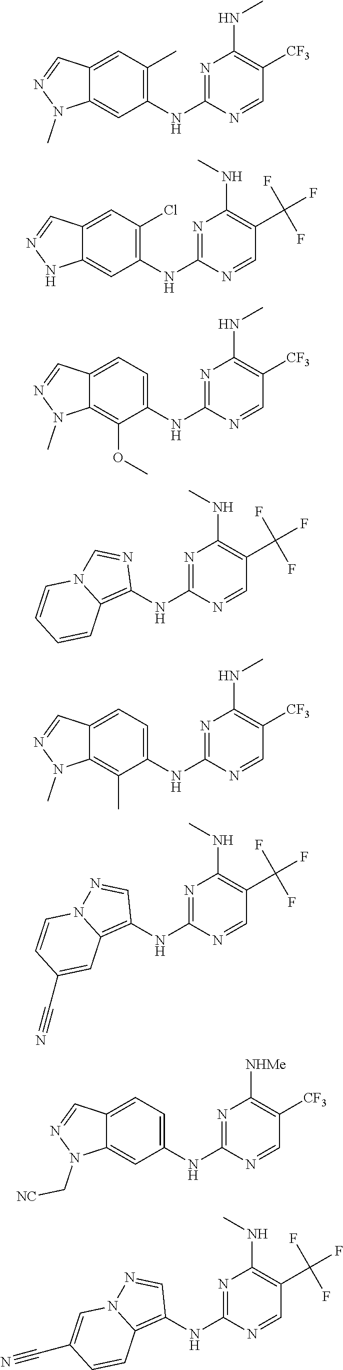

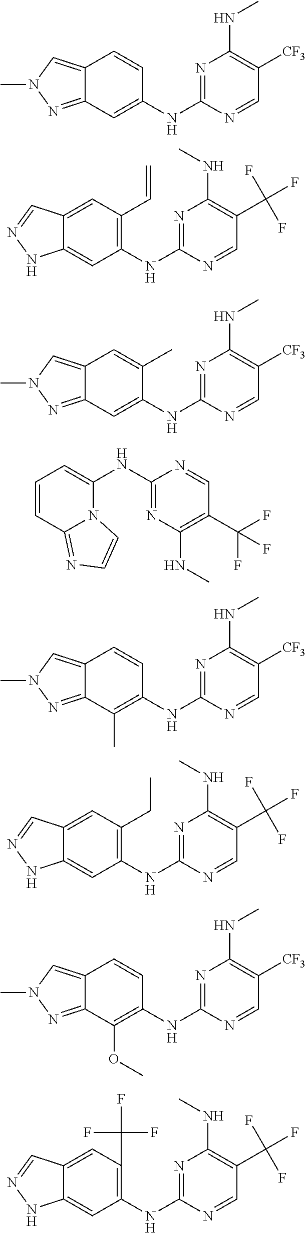

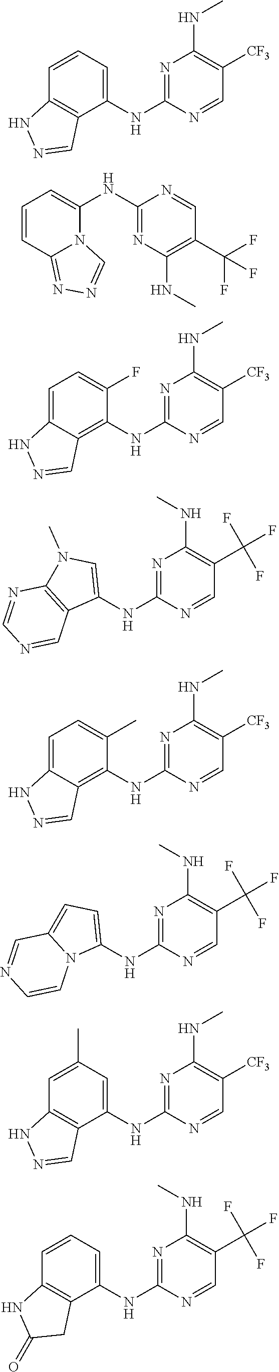

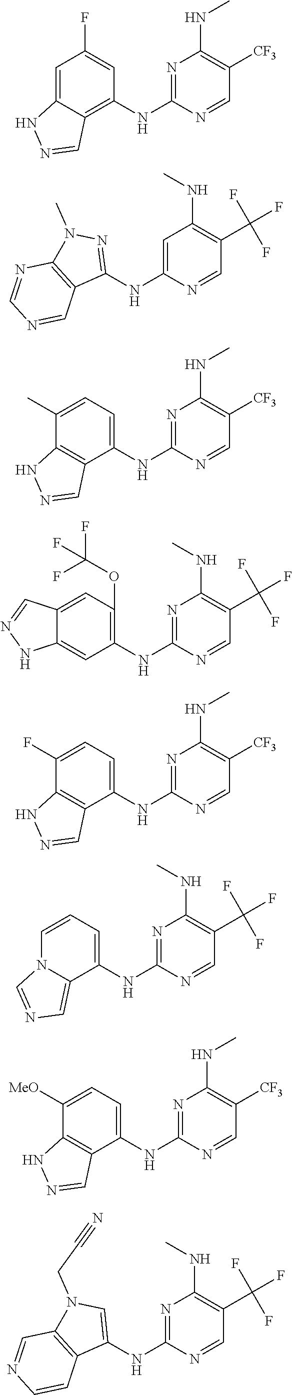

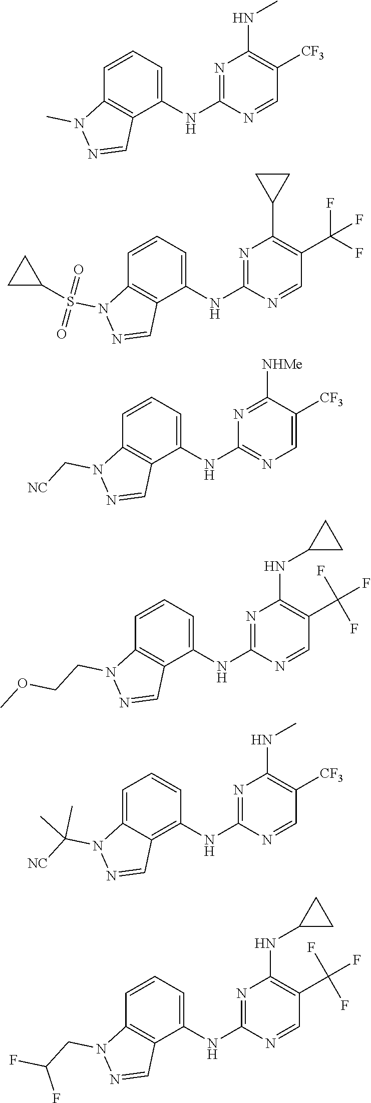 [SMILES: CNc1nc(Nc2cccc3c2cnn3C)ncc1C(F)(F)F ; O=S(=O)(C1CC1)n1ncc2c(Nc3ncc(C(F)(F)F)c(C4CC4)n3)cccc21 ; CNc1nc(Nc2cccc3c2cnn3CC#N)ncc1C(F)(F)F ; COCCn1ncc2c(Nc3ncc(C(F)(F)F)c(NC4CC4)n3)cccc21 ; CNc1nc(Nc2cccc3c2cnn3C(C)(C)C#N)ncc1C(F)(F)F ; FC(F)Cn1ncc2c(Nc3ncc(C(F)(F)F)c(NC4CC4)n3)cccc21]

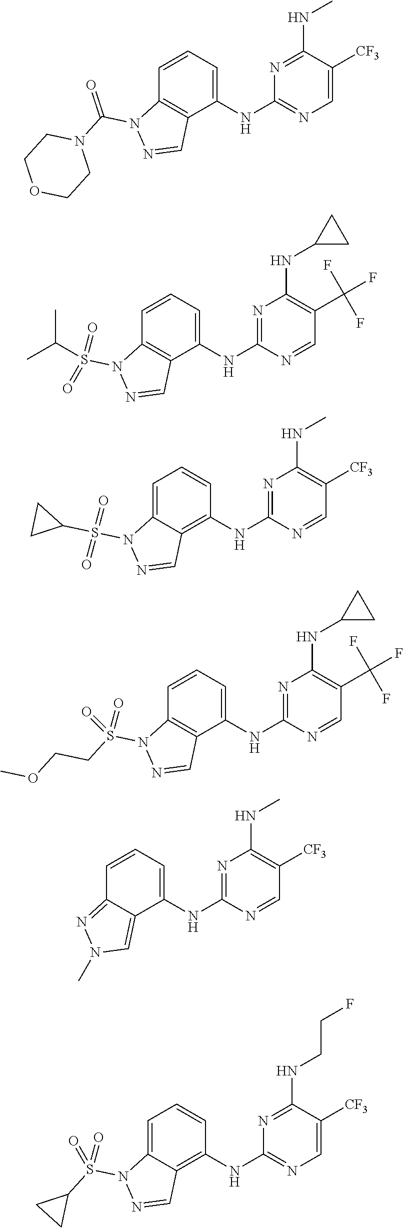 [SMILES: CNc1nc(Nc2cccc3c2cnn3C(=O)N2CCOCC2)ncc1C(F)(F)F ; CC(C)S(=O)(=O)n1ncc2c(Nc3ncc(C(F)(F)F)c(NC4CC4)n3)cccc21 ; CNc1nc(Nc2cccc3c2cnn3S(=O)(=O)C2CC2)ncc1C(F)(F)F ; COCCS(=O)(=O)n1ncc2c(Nc3ncc(C(F)(F)F)c(NC4CC4)n3)cccc21 ; CNc1nc(Nc2cccc3nn(C)cc23)ncc1C(F)(F)F ; O=S(=O)(C1CC1)n1ncc2c(Nc3ncc(C(F)(F)F)c(NCCF)n3)cccc21]

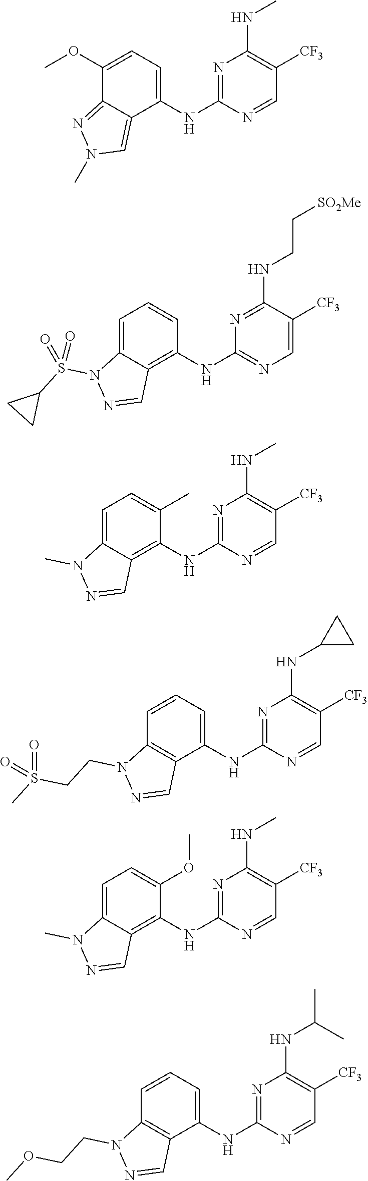

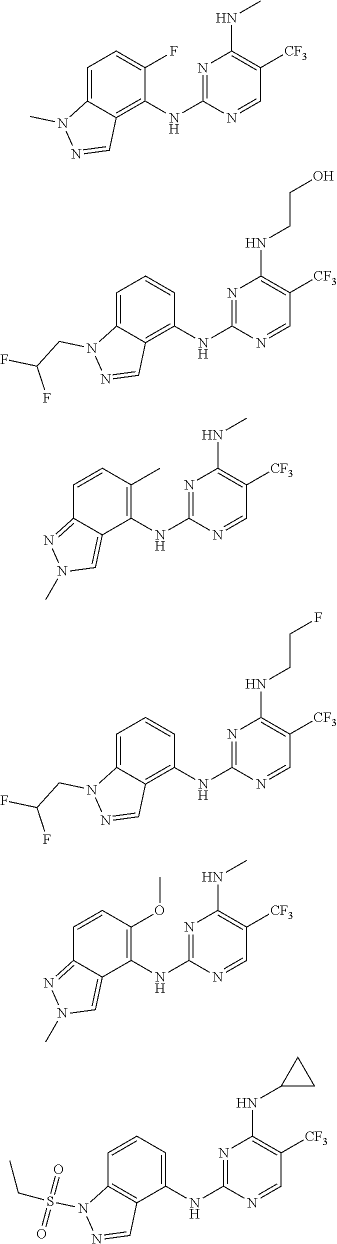 [SMILES: CNc1nc(Nc2c(F)ccc3c2cnn3C)ncc1C(F)(F)F ; OCCNc1nc(Nc2cccc3c2cnn3CC(F)F)ncc1C(F)(F)F ; CNc1nc(Nc2c(C)ccc3nn(C)cc23)ncc1C(F)(F)F ; FCCNc1nc(Nc2cccc3c2cnn3CC(F)F)ncc1C(F)(F)F ; CNc1nc(Nc2c(OC)ccc3nn(C)cc23)ncc1C(F)(F)F ; CCS(=O)(=O)n1ncc2c(Nc3ncc(C(F)(F)F)c(NC4CC4)n3)cccc21]

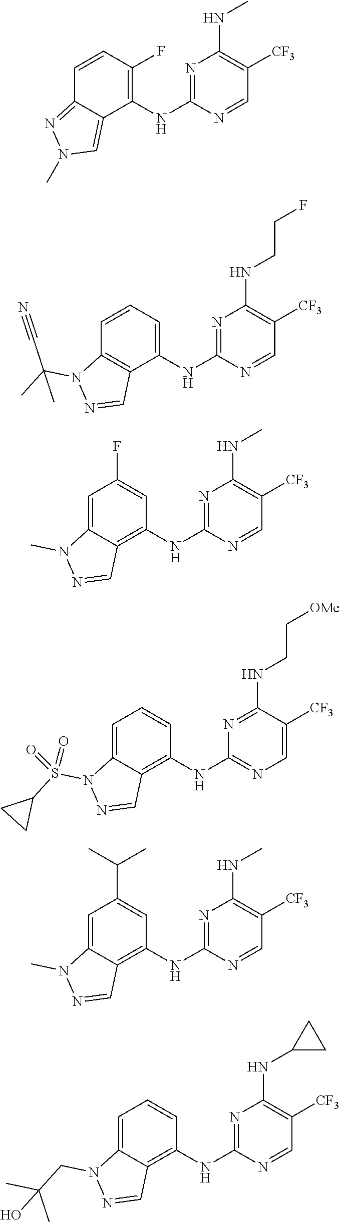

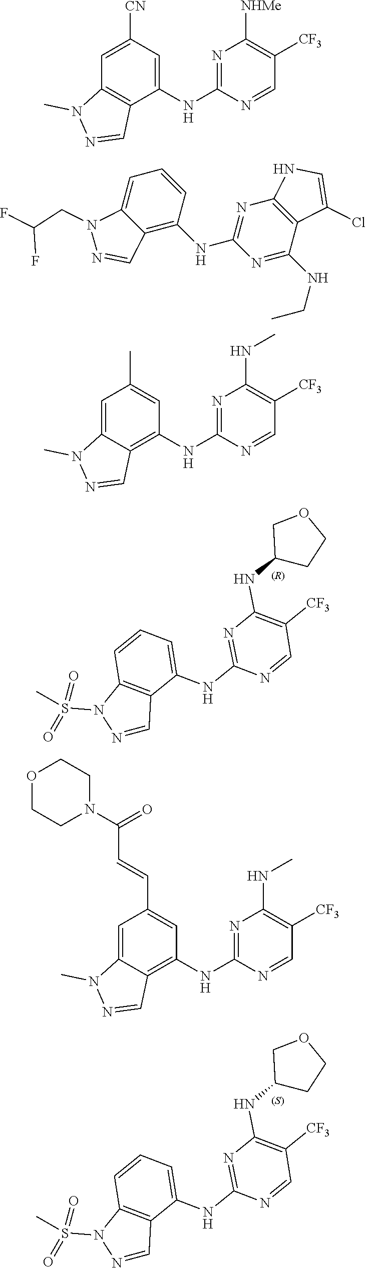

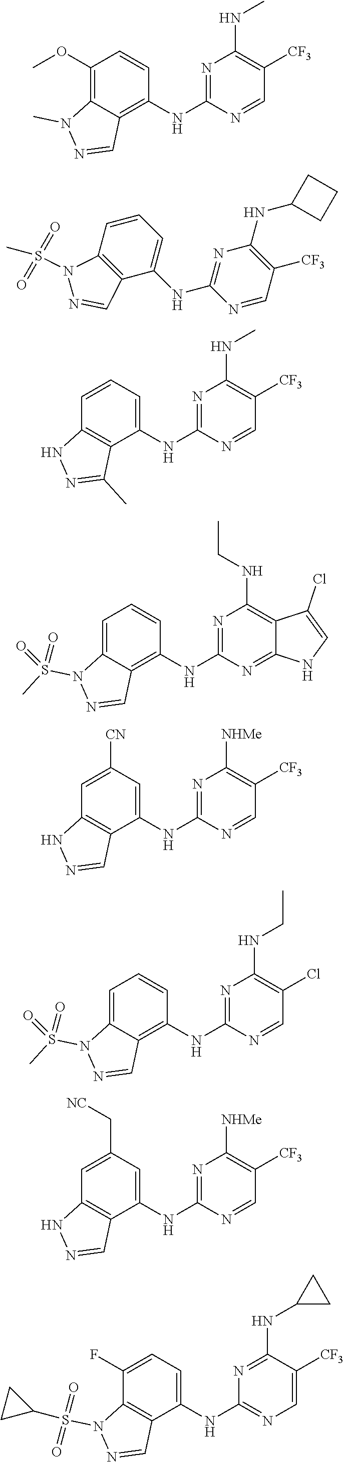

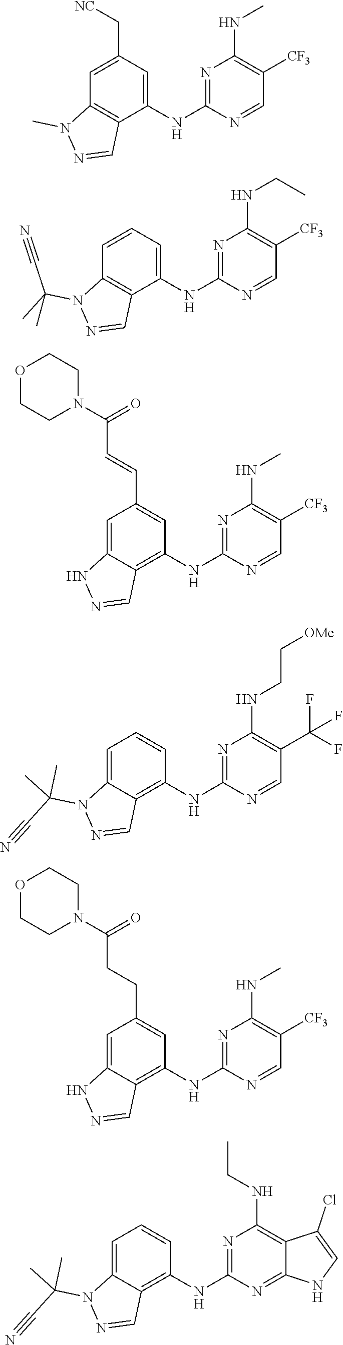

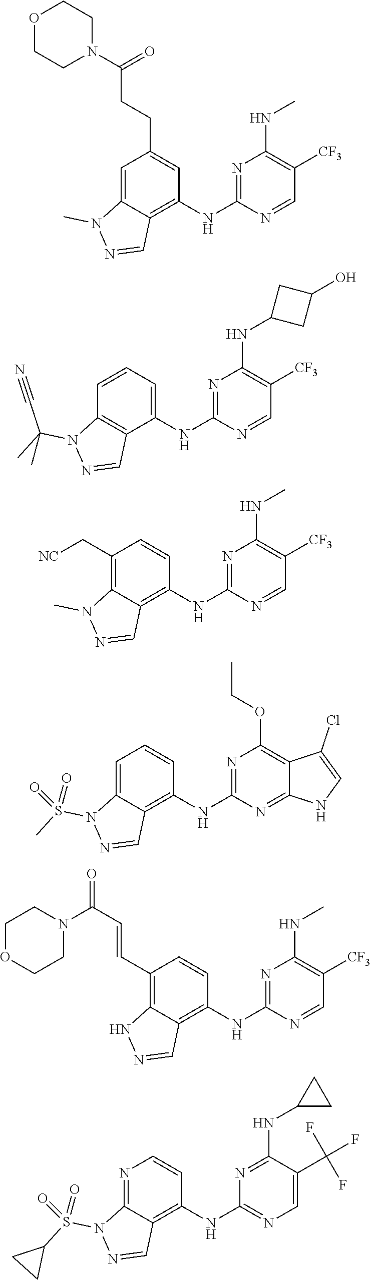

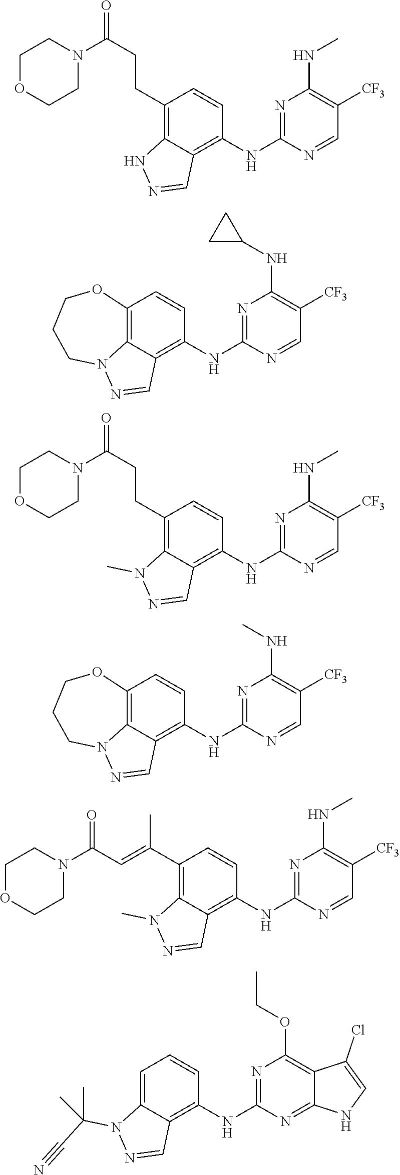

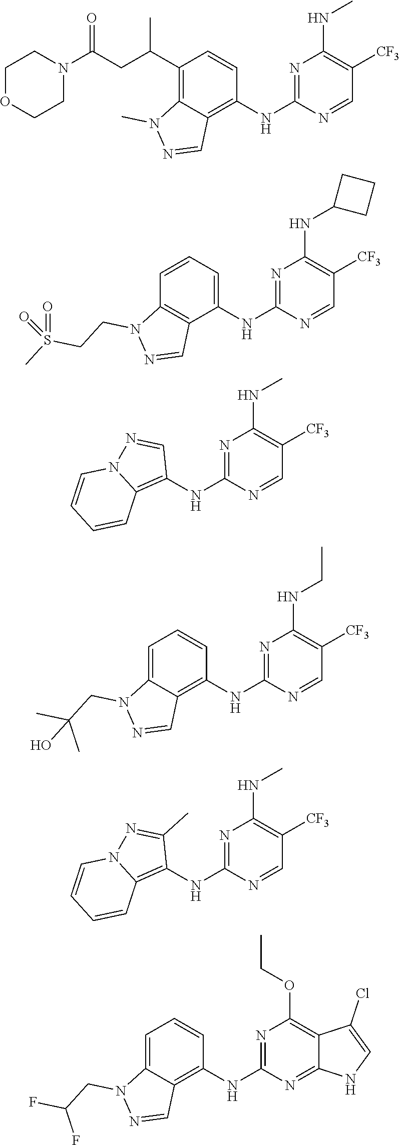 [SMILES: CNc1nc(Nc2ccc(C(C)CC(=O)N3CCOCC3)c3c2cnn3C)ncc1C(F)(F)F ; CS(=O)(=O)CCn1ncc2c(Nc3ncc(C(F)(F)F)c(NC4CCC4)n3)cccc21 ; CNc1nc(Nc2cnn3ccccc23)ncc1C(F)(F)F ; CCNc1nc(Nc2cccc3c2cnn3CC(C)(C)O)ncc1C(F)(F)F ; CNc1nc(Nc2c(C)nn3ccccc23)ncc1C(F)(F)F ; CCOc1nc(Nc2cccc3c2cnn3CC(F)F)nc2[nH]cc(Cl)c12]

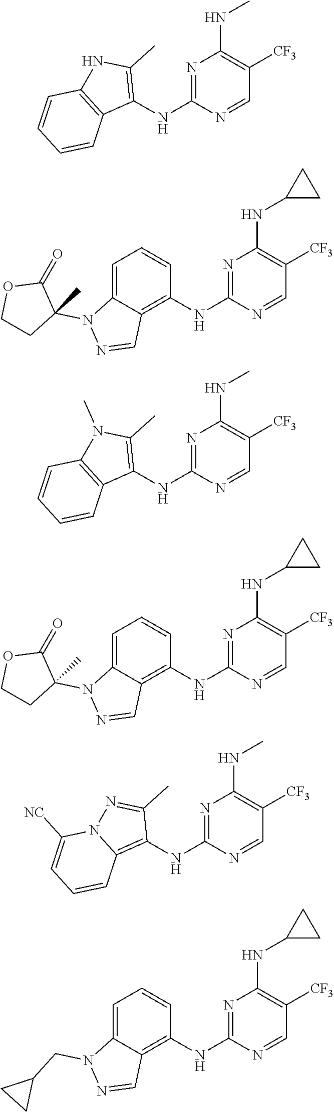 [SMILES: CNc1nc(Nc2c(C)[nH]c3ccccc23)ncc1C(F)(F)F ; C[C@@]1(n2ncc3c(Nc4ncc(C(F)(F)F)c(NC5CC5)n4)cccc32)CCOC1=O ; CNc1nc(Nc2c(C)n(C)c3ccccc23)ncc1C(F)(F)F ; C[C@]1(n2ncc3c(Nc4ncc(C(F)(F)F)c(NC5CC5)n4)cccc32)CCOC1=O ; CNc1nc(Nc2c(C)nn3c(C#N)cccc23)ncc1C(F)(F)F ; FC(F)(F)c1cnc(Nc2cccc3c2cnn3CC2CC2)nc1NC1CC1]

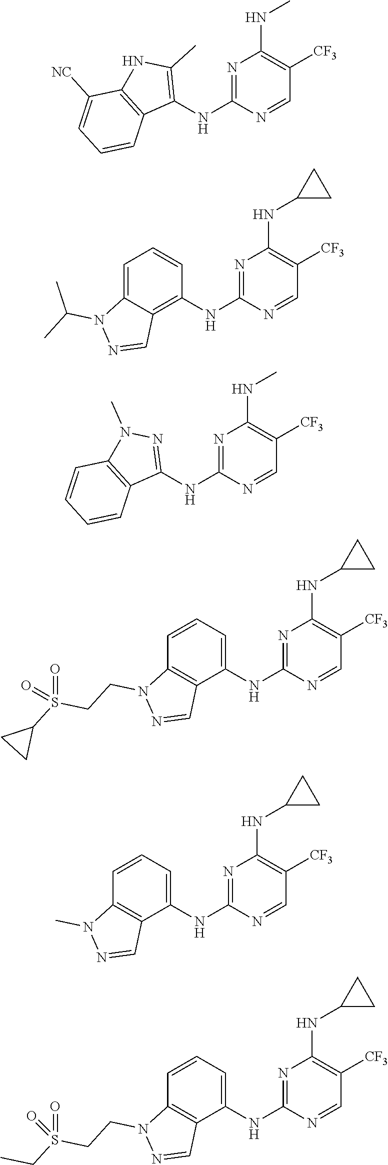 [SMILES: CNc1nc(Nc2c(C)[nH]c3c(C#N)cccc23)ncc1C(F)(F)F ; CC(C)n1ncc2c(Nc3ncc(C(F)(F)F)c(NC4CC4)n3)cccc21 ; CNc1nc(Nc2nn(C)c3ccccc23)ncc1C(F)(F)F ; O=S(=O)(CCn1ncc2c(Nc3ncc(C(F)(F)F)c(NC4CC4)n3)cccc21)C1CC1 ; Cn1ncc2c(Nc3ncc(C(F)(F)F)c(NC4CC4)n3)cccc21 ; CCS(=O)(=O)CCn1ncc2c(Nc3ncc(C(F)(F)F)c(NC4CC4)n3)cccc21]

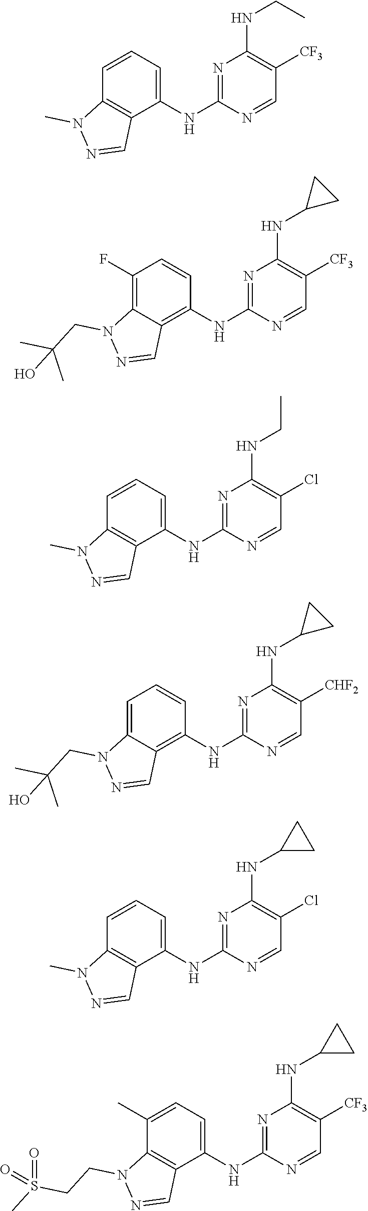 [SMILES: CCNc1nc(Nc2cccc3c2cnn3C)ncc1C(F)(F)F ; CC(C)(O)Cn1ncc2c(Nc3ncc(C(F)(F)F)c(NC4CC4)n3)ccc(F)c21 ; CCNc1nc(Nc2cccc3c2cnn3C)ncc1Cl ; CC(C)(O)Cn1ncc2c(Nc3ncc(C(F)F)c(NC4CC4)n3)cccc21 ; Cn1ncc2c(Nc3ncc(Cl)c(NC4CC4)n3)cccc21 ; Cc1ccc(Nc2ncc(C(F)(F)F)c(NC3CC3)n2)c2cnn(CCS(C)(=O)=O)c12]

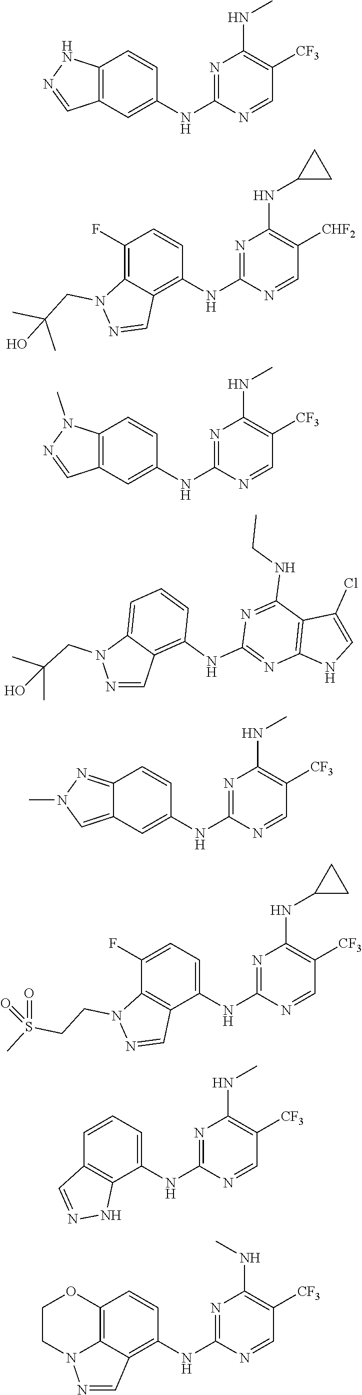

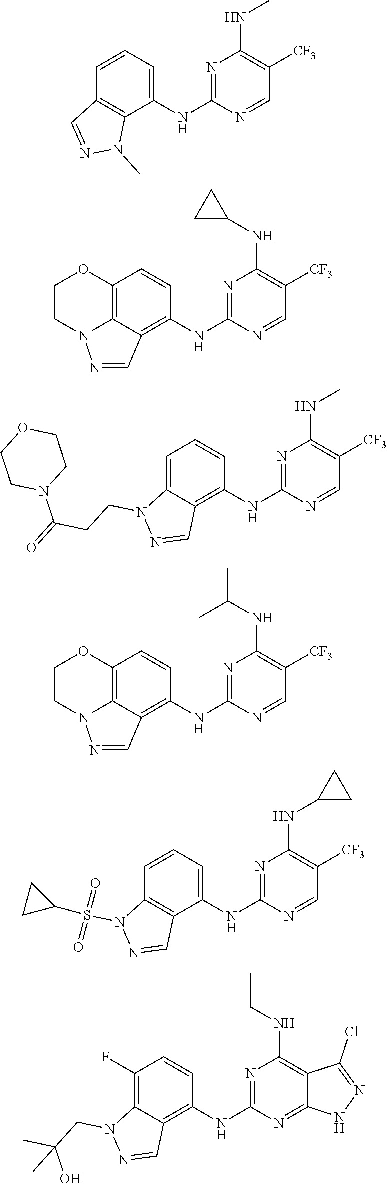 [SMILES: CNc1nc(Nc2cccc3cnn(C)c23)ncc1C(F)(F)F ; FC(F)(F)c1cnc(Nc2ccc3c4c2cnn4CCO3)nc1NC1CC1 ; CNc1nc(Nc2cccc3c2cnn3CCC(=O)N2CCOCC2)ncc1C(F)(F)F ; CC(C)Nc1nc(Nc2ccc3c4c2cnn4CCO3)ncc1C(F)(F)F ; O=S(=O)(C1CC1)n1ncc2c(Nc3ncc(C(F)(F)F)c(NC4CC4)n3)cccc21 ; CCNc1nc(Nc2ccc(F)c3c2cnn3CC(C)(C)O)nc2[nH]nc(Cl)c12]

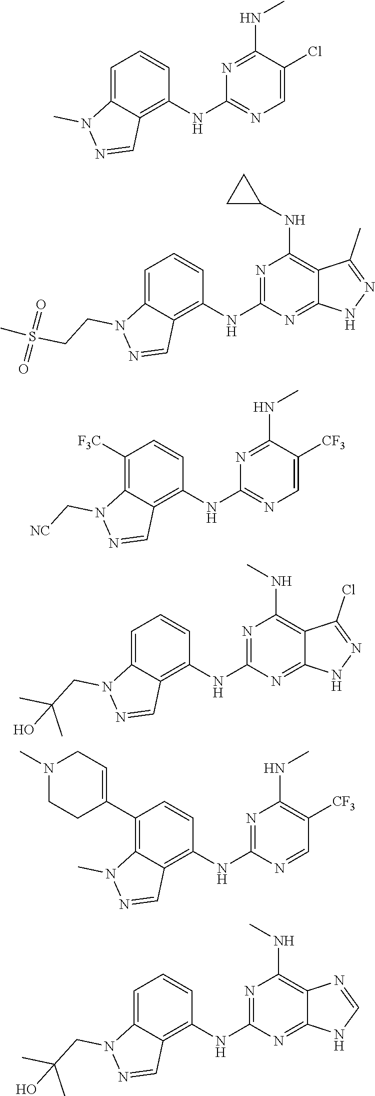

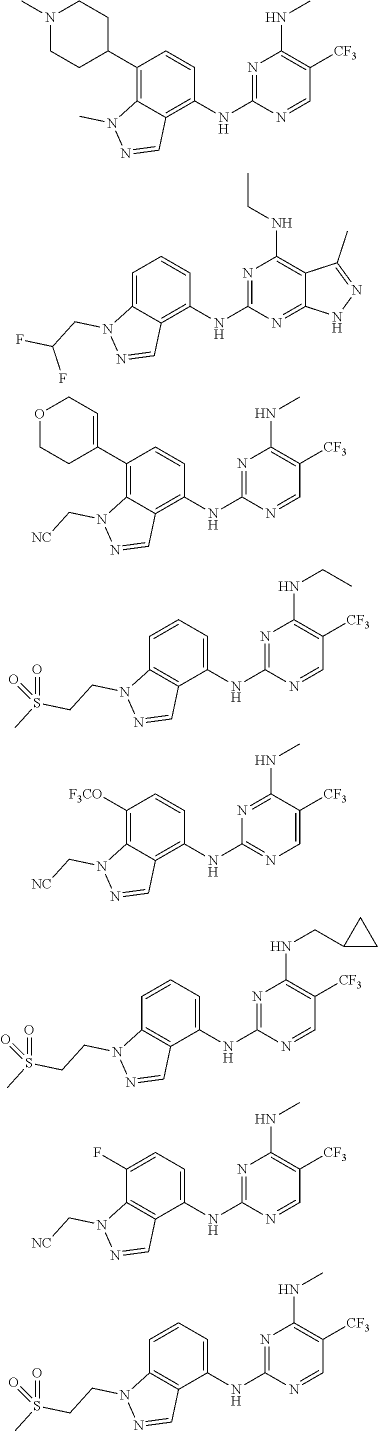

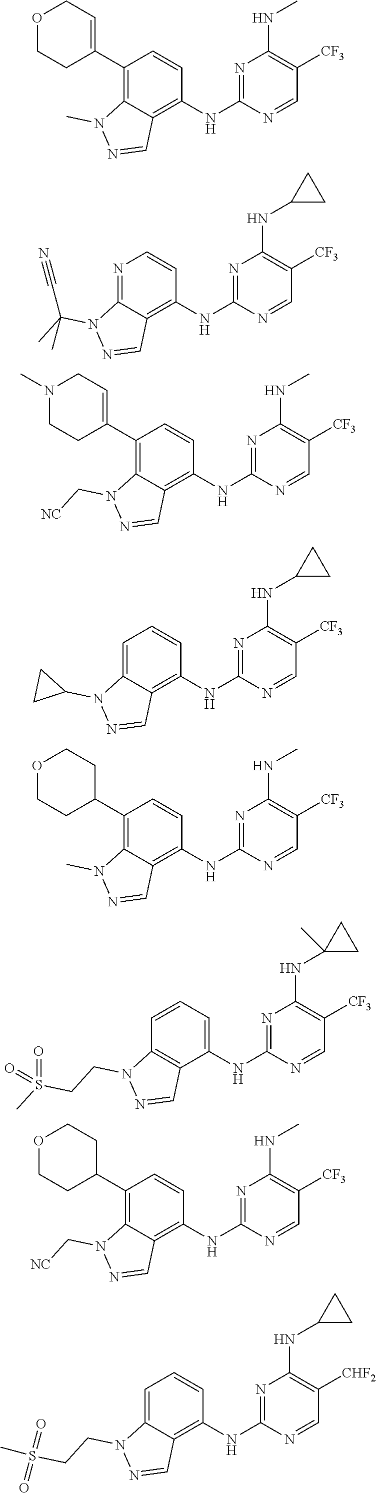

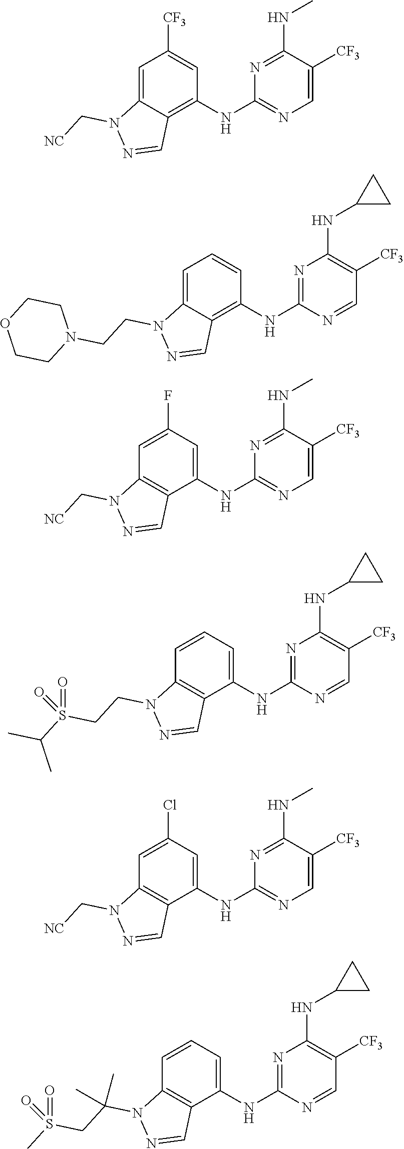

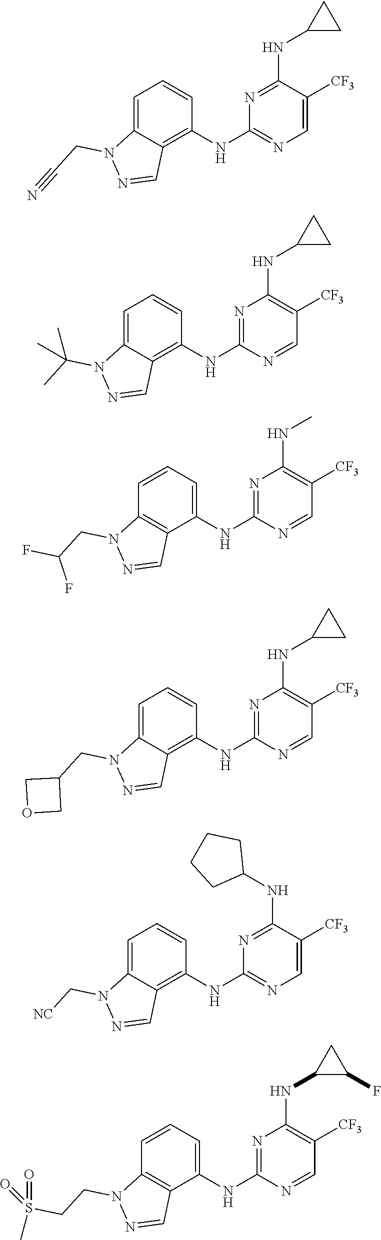

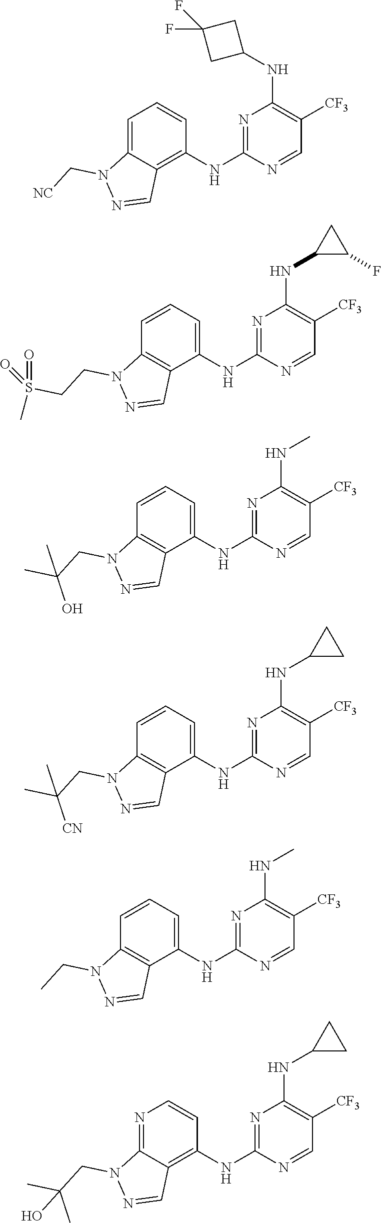

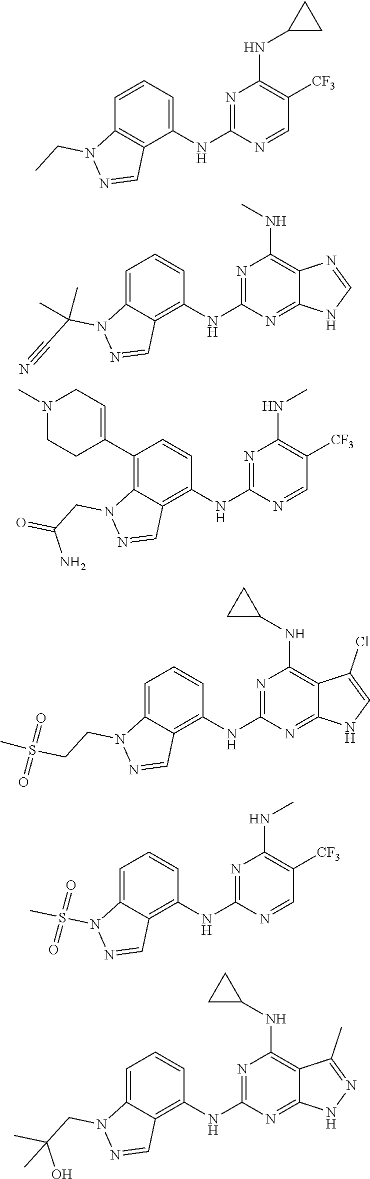

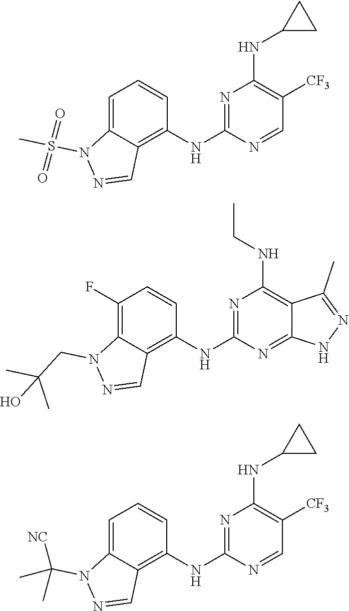 [SMILES: CS(=O)(=O)n1ncc2c(Nc3ncc(C(F)(F)F)c(NC4CC4)n3)cccc21 ; CCNc1nc(Nc2ccc(F)c3c2cnn3CC(C)(C)O)nc2[nH]nc(C)c12 ; CC(C)(C#N)n1ncc2c(Nc3ncc(C(F)(F)F)c(NC4CC4)n3)cccc21]

In one embodiment, the invention provides a pharmaceutical composition comprising a compound of the above Formulas (I)-(IV) and pharmaceutically acceptable salts thereof together with a pharmaceutically acceptable excipient.

In one embodiment, the invention provides compounds of the above Formulas (I)-(IV) and pharmaceutically acceptable salts thereof for use in therapy.

In one embodiment, the invention provides compounds of the above Formulas (I)-(V) and pharmaceutically acceptable salts thereof for use in a method for the treatment of a disease associated with LRRK2. The disease may be a neurological condition.

In one embodiment, the invention relates to the use of a compound of the above Formulas (I)-(IV) and pharmaceutically acceptable salts thereof in the manufacture of a medicament for use in the treatment of a disease associated with LRRK2.

In one embodiment, the invention relates to a method for the treatment of a disease associated with LRRK2, including PD, the method comprising the administration of a therapeutically effective amount of a compound of the above Formulas (I)-(IV) and pharmaceutically acceptable salts thereof to a patient in need thereof.

In another aspect, the invention provides methods of modulating the activity of a kinase by contacting cells containing a kinase with one or more compounds of the invention, such as any of those described above. The compound may inhibit activity of the kinase. The compound may increase activity of the kinase. The kinase may be LRRK2.

In embodiments of the use, the condition treated by the compounds of the invention is an autoimmune disease, inflammatory disease, bone disease, metabolic disease, neurological or neurodegenerative disease, cancer, cardiovascular disease, allergies, asthma, Alzheimer's disease, Parkinson's disease, skin disorder, eye disease, infectious disease, or hormone-related disease.

### Pharmaceutical Compositions

The present invention provides pharmaceutical compositions containing one or more compounds described above, or a pharmaceutically acceptable ester, prodrug, hydrate, solvate or salt of such a compound, optionally in combination with a pharmaceutically acceptable carrier. The invention further provides such compounds for the preparation of a medicament for the treatment of one or more diseases mentioned herein.

A pharmaceutical composition may contain one or more compounds of the invention in a therapeutically effective amount. A therapeutically effective amount of a compound in accordance with this invention means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage may be adjusted to the individual requirements in each particular case including the specific compound being administered, the route of administration, the condition being treated, as well as the patient being treated.

Compositions of the invention may include a vehicle for delivery of one or more compounds of the invention. For example, the composition may contain particles, such as nanoparticles, microparticles, liposomes, micelles, and virus particles.

Examples of pharmacologically acceptable salts of sufficiently basic compounds of the invention are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound of the invention may form alkali or earth alkali metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts; all of which are also further examples of salts of the invention. Compounds of the invention may be solvated, especially hydrated. The hydratization/hydration may occur during the process of production or as a consequence of the hygroscopic nature of the initially water free compounds of the invention. The solvates and/or hydrates may e.g. be present in solid or liquid form.

It should be appreciated that certain compounds of the invention may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). All these tautomeric forms, geometrical or optical isomers (as well as racemates and diastereomers) and polymorphous forms are included in the invention. Since the compounds of the invention may contain asymmetric C-atoms, they may be present either as achiral compounds, mixtures of diastereomers, mixtures of enantiomers or as optically pure compounds. The present invention comprises both all pure enantiomers and all pure diastereomers, and also the mixtures thereof in any mixing ratio.

According to a further embodiment of the present invention, one or more hydrogen atoms of the compounds of the present invention may be replaced by deuterium. Deuterium modification improves the metabolic properties of a drug with little or no change in its intrinsic pharmacology. Deuterium substitution at specific molecular positions improves metabolic stability reduces formation of toxic metabolites and/or increases the formation of desired active metabolites. Accordingly, the present invention also encompasses the partially and fully deuterated compounds of the invention. The term hydrogen also encompasses deuterium.

The therapeutic use of compounds according to the invention, their pharmacologically acceptable salts, solvates and hydrates, respectively, as well as formulations and pharmaceutical compositions also lie within the scope of the present invention. The pharmaceutical compositions according to the present invention may comprise at least one compound of the invention as an active ingredient and, optionally, carrier substances and/or adjuvants.

The present invention also relates to prodrugs which are composed of a compound of the invention and at least one pharmacologically acceptable protective group which will be cleaved off under physiological conditions, such as an alkoxy-, arylalkyloxy-, acyl-, acyloxymethyl group (e.g. pivaloyloxymethyl), an 2-alkyl-, 2-aryl- or 2-arylalkyl oxycarbonyl-2-alkylidene ethyl group or an acyloxy group as defined herein, e.g. ethoxy, benzyloxy, acetyl or acetyloxy or, especially for a compound of the invention, carrying a hydroxy group (—OH): a sulfate, a phosphate (—OPO₃ or —OCH₂OPO₃) or an ester of an amino acid. For example, compositions may contain pro-drugs of the hydroxy group of a compound of the invention.

As used herein, the term pharmaceutically acceptable ester especially refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The present invention also relates to a prodrug, a biohydrolyzable ester, a biohydrolyzable amide, a polymorph, tautomer, stereoisomer, metabolite, N-oxide, biohydrolyzable carbamate, biohydrolyzable ether, physiologically functional derivative, atropisomer, or in vivo-hydrolysable precursor, diastereomer or mixture of diastereomers, chemically protected form, affinity reagent, complex, chelate and a stereoisomer of the compounds of the invention.

As mentioned above, therapeutically useful agents that contain compounds of the invention, their solvates, salts or formulations are also comprised in the scope of the present invention. In general, compounds of the invention will be administered by using the known and acceptable modes known in the art, either alone or in combination with any other therapeutic agent.

For oral administration such therapeutically useful agents can be administered by one of the following routes: oral, e.g. as tablets, dragees, coated tablets, pills, semisolids, soft or hard capsules, for example soft and hard gelatin capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containing the active ingredient or intranasal. For the production of such tablets, pills, semisolids, coated tablets, dragees and hard, e.g. gelatin capsules, the therapeutically useful product may be mixed with pharmaceutically inert, inorganic or organic excipients as are e.g. lactose, sucrose, glucose, gelatine, malt, silica gel, starch or derivatives thereof, tale, stearinic acid or their salts, dried skim milk, and the like. For the production of soft capsules one may use excipients as are e.g. vegetable, petroleum animal or synthetic oils, wax, fat, polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Particularly useful are lipids, such as phospholipids (e.g., natural origin and/or with a particle size between 300 to 350 nm) in phosphate buffered saline (pH=7 to 8, e.g., 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilization, e.g. UV stabilizers, emulsifiers, sweetener, aromatizers, salts to change the osmotic pressure, buffers, coating additives and antioxidants.

In general, in the case of oral or parenteral administration to adult humans weighing approximately 80 kg, a daily dosage of about 10 mg to about 10,000 mg, or from about 20 mg to about 1,000 mg, should be appropriate, although the upper limit may be exceeded when indicated.

The daily dosage can be administered as a single dose or in divided doses, or for parenteral administration, it may be given as continuous infusion or subcutaneous injection.

### Methods of Making Compounds

The invention also provides methods of making compounds of the invention, such as those described above. Synthesis schemes for making specific compounds of Formula (I) are provided in the Examples below.

### Methods of Treating Conditions

The compounds and compositions of the invention modulate activity of one or more protein kinases. The compounds and compositions may inhibit, activate, or otherwise alter kinase activity. Consequently, the compounds and compositions may be used to diagnose, treat, or prevent a condition, such as a disease, disorder, or other condition for which modulation of kinase activity provides therapeutic benefit.

Diseases, disorders, and conditions that can be diagnosed and/or treated using compositions and methods of the invention include those associated with aberrant activity, e.g., increased activity or decreased activity, of one or more kinases. The disease, disorder, or condition may be associated with aberrant LRRK2 activity, such as Alzheimer's disease, Crohn's disease, inflammatory bowel disease, an inflammatory disease, leprosy, neurodegenerative diseases, a non-skin cancer, or Parkinson's disease, including familial Parkinson's disease, sporadic Parkinson's disease, late-onset Parkinson's disease (PD), and type 8 Parkinson's disease.

The disease, disorder, or condition may be or include a respiratory tract/obstructive airways disease or disorder, such as rhinorrhea, tracheal constriction, airway contraction, acute-, allergic, atrophic rhinitis or chronic rhinitis (such as rhinitis caseosa, hypertrophic rhinitis, rhinitis purulenta, rhinitis sicca), rhinitis medicamentosa, membranous rhinitis (including croupous, fibrinous and pseudomembranous rhinitis), scrofulous rhinitis, perennial allergic rhinitis, seasonal rhinitis (including rhinitis nervosa (hay fever) and vasomotor rhinitis), pollinosis, asthma (such as bronchial, atopic, allergic, intrinsic, extrinsic, exercise-induced, cold air-induced, occupational, bacterial infection-induced, and dust asthma particularly chronic or inveterate asthma (e.g. late asthma and airways hyper-responsiveness)), bronchitis (including chronic, acute, arachidic, catarrhal, croupus, phthinoid and eosinophilic bronchitis), cardiobronchitis, pneumoconiosis, chronic inflammatory disease of the lung which result in interstitial fibrosis, such as interstitial lung disease (ILD) (e.g., idiopathic pulmonary fibrosis, or ILD associated with rheumatoid arthritis, or other autoimmune conditions), acute lung injury (ALI), adult respiratory distress syndrome (ARDS), chronic obstructive pulmonary, airways or lung disease (CORD, COAD, COLD or COPD, such as irreversible COPD), chronic sinusitis, conjunctivitis (e.g. allergic conjunctivitis), cystic fibrosis, extrinsic allergic alveolitis (like farmer's lung and related diseases), fibroid lung, hypersensitivity lung diseases, hypersensitivity pneumonitis, idiopathic interstitial pneumonia, nasal congestion, nasal polyposis, otitis media, and cough (chronic cough associated with inflammation or iatrogenic induced), pleurisy, pulmonary congestion, emphysema, bronchiectasis, sarcoidosis, lung fibrosis, including cryptogenic fibrosing alveolitis, fibrosis complicating anti-neoplastic therapy and chronic infection, including tuberculosis and aspergillosis and other fungal infections, vasculitic and thrombotic disorders of the lung vasculature, and pulmonary hypertension, acute viral infection including the common cold, and infection due to respiratory syncytial virus, influenza, coronavirus (including SARS) and adenovirus, allergic bronchopulmonary mycosis, emphysema, diffuse panbronchiolitis, systemic anaphylaxis or hypersensitivity responses, drug allergies (e.g., to penicillin, cephalosporins), insect sting allergies, and food related allergies which may have effects remote from the gut (such as migraine, rhinitis and eczema), anaphylactic shock, or vascular spasms.

The disease, disorder, or condition may be or include a bone and joint related disease or disorder, such as osteoporosis, arthritis (including rheumatic, infectious, autoimmune, chronic, rnalignant), seronegative spondyloarthropathies (such as ankylosing spondylitis, rheumatoid spondylitis, psoriatic arthritis, enthesopathy, Bechet's disease, Marie-Strumpell arthritis, arthritis of inflammatory bowel disease, and Reiter's disease), systemic sclerosis, osteoarthritis, osteoarthrosis, both primary and secondary to e.g. congenital hip dysplasia, cervical and lumbar spondylitis, and low back and neck pain, Still's disease, reactive arthritis and undifferentiated spondarthropathy, septic arthritis and other infection-related arthropathies and bone disorders such as tuberculosis, including Pott's disease and Poncet's syndrome, acute and chronic crystal-induced synovitis including urate gout, calcium pyrophosphate deposition disease, and calcium apatite related tendon, bursar and synovial inflammation, primary and secondary Sjogren's syndrome, systemic sclerosis and limited scleroderma, mixed connective tissue disease, and undifferentiated connective tissue disease, inflammatory myopathies including, polymalgia rheumatica, juvenile arthritis including idiopathic inflammatory arthritides of whatever joint distribution and associated syndromes, other joint disease (such as intervertebral disc degeneration or temporomandibular joint degeneration), rheumatic fever and its systemic complications, vasculitides including giant cell arteritis, Takayasu's arteritis, polyarteritis nodosa, microscopic polyarteritis, and vasculitides to associated with viral infection, hypersensitivity reactions, cryoglobulins, paraproteins, low back pain, Familial Mediterranean fever, Muckle-Wells syndrome, and Familial Hibenian Fever, Kikuchi disease, drug-induced arthalgias, tendonititides, polychondritis, and myopathies, osteoporosis, osteomalacia like osteoporosis, osteopenia, osteogenesis imperfects, osteopetrosis, osteofibrosis, osteonecrosis, Paget's disease of bone, hypophosphatemia, Felty's syndrome, Still's disease, slack of artificial joint implant, sprain or strain of muscle or joint, tendinitis, fasciitis, periarthritis humeroscapularis, cervico-omo-brachial syndrome, or tenosynovitis.

The disease, disorder, or condition may be or include a skin or eye related disease or disorder, such as glaucoma, ocular hypertension, cataract, retinal detachment, psoriasis (including psoriasis vulgaris, pustular psoriasis, arthritic psoriasis, erythroderma psoriaticum), palmoplantar pustulosis, xerodoma, eczematous diseases (like atopic dermatitis, ultraviolet radiation dermatitis, contact dermatitis, and seborrheic dermatitis), phytodermatitis, photodermatitis, cutaneous eosinophilias, chronic skin ulcers, cutaneous lupus erythematosus, contact hypersensitivity/allergic contact dermatitis (including sensitivity to poison ivy, sumac, or oak), and eosinophilic folliculitis (Ofuji's disease), pruritus, drug eruptions, urticaria (acute or chronic, allergic or non-allergic), acne, erythema, dermatitis herpetiformis, scleroderma, vitiligo, lichen planus, lichen sclerosus et atrophica, pyodenna gangrenosum, skin sarcoid, pemphigus, ocular pemphigus, pemphigoid, epidermolysis bullosa, angioedema, vasculitides, toxic erythemas, cutaneous eosinophilias, alopecia areata, male-pattern baldness, Sweet's syndrome, Stevens-Johnson syndrome, Weber-Christian syndrome, erythema multiforme, cellulitis, both, infective and non infective, panniculitis, cutaneous Lymphomas, non-r melanoma skin cancer and other dysplastic lesions, blepharitis, iritis, anterior and posterior uveitis, choroiditis, autoimmune, degenerative or inflammatory disorders affecting the retina, ophthalmitis including sympathetic ophthalmitis, sarcoidosis, xerosis infections including viral, fungal, and bacterial, allergic conjunctivitis, increased fibrosis, keloids, keloplasty, post surgical scars, epidermolysis bullosa, dry eye, ocular inflammation, allergic conjunctivitis, vernal conjunctivitis, vernal keratoconjunctivitis, and giant papillary conjunctivitis, ocular angiogenesis, cornea damage and scar, all forms of macular degeneration, macular edema, macular dystrophy, abnormal wound healing, scleritis, episcleritis, pachydermia, peripheral ulcerative keratitis, fungal keratitis, herpetic keratitis, invasive aspergillosis; conical cornea, dystorphia epithelialis comeae, or severe intraocular inflammation.

The disease, disorder, or condition may be or include a gastrointestinal tract and abdominal related disease or disorder, such as celiac/coeliac disease (e.g. celiac sprue), cholecystitis, enteritis (including infectious, ischemic, radiation, drug-induced, and eosinophilic gastroenteritis), eosinophilic esophagitis, eosinophilic gastrointestinal inflammation, allergen induced diarrhea, enteropathy associated with seronegative arthropathies, gastritis, autoimmune atrophic gastritis, ischemic bowel disease, inflammatory bowel disease (Crohn's disease and ulcerative colitis), colitis, Mooren's ulcer, irritable bowel syndrome, necrotizing enterocolitis, gut ischemia, glossitis, gingivitis, periodontitis, oesophagitis, including reflex, proctitis, fibrosis and cirrhosis of the liver, pancreatitis, both acute and chronic, pancreatic fibrosis, pancreatic sclerosis, pancreatolithiasis, hepatic cirrhosis, hepatitis (congestive, autoimmune, acute, fulminant, chronic, drug-induced, alcoholic, lupoid, steatohepatitis and chronic viral), fatty liver, primary biliary cirrhosis, hepatic porphyria, and gastrointestinal related allergic disorders, spastic colon, diverticulitis, gastroenteric bleeding, Behcet's disease; partial liver resection, acute liver necrosis (e.g. necrosis caused by toxins, viral hepatitis, shock or anoxia), or hemolytic uremic syndrome.

The disease, disorder, or condition may be or include a hematological disease or disorder, such as anemias, coagulation, myeloproliferative disorders, hemorrhagic disorders, leukopenia, eosinophilic disorders, leukemias (e.g. myelogenous, lymphomas, plasma cell dyscrasias, disorders of the spleen, Band's disease, hemophilia, purpura (including idiopathic thrombocytopenic purpura), or Wiskott-Aldrich syndrome.

The disease, disorder, or condition may be or include a metabolic disease or disorder, such as obesity, amyloidosis, disturbances of the amino and acid metabolism like branched chain disease, hyperaminoacidemia, hyperaminoaciduria, disturbances of the metabolism of urea, hyperammonemia, mucopolysaccharidoses e.g. Maroteaux-Lamy syndrome, storage disease like glycogen storage diseases and lipid storage diseases, glycogenosis I diseases like Cori's disease, malabsorption diseases like intestinal carbohydrate malabsorption, oligosaccharidase deficiency like maltase-, lactase-, sucrase-insufficiency, disorders of the metabolism of fructose, disorders of the metabolism of galactose, galactosaemia, disturbances of carbohydrate utilization like diabetes, hypoglycemia, disturbances of pyruvate metabolism, hypolipidemia, hypolipoproteinemia, hyperlipidemia, hyperlipoproteinemia, carnitine or carnitine acyltransferase deficiency, disturbances of the porphyrin metabolism, porphyrins, disturbances of the purine metabolism, lysosomal diseases, metabolic diseases of nerves and nervous systems like gangliosidoses, sphingolipidoses, sulfatidoses, leucodystrophies, or Lesch Nyhan syndrome.

The disease, disorder, or condition may be or include a cerebellar dysfunction or disturbance of brain metabolism, such as dementia, Alzheimer's disease, Huntington's chores, Parkinson's disease, Pick's disease, toxic encepha-lopathy, demyelinating neuropathies like inflammatory neuropathy, Guillain-Barre syndrome; Meniere's disease and radiculopathy, primary and secondary metabolic disorders associated with hormonal defects like any disorder stemming from either an hyperfunction or hypofunction of some hormone-secreting endocrine gland and any combination thereof. Sipple's syndrome, pituitary gland dysfunction and its effects on other endocrine glands, such as the thyroid, adrenals, ovaries, and testes, acromegaly, hyper- and hypothyroidism, euthyroid goiter, euthyroid sick syndrome, thyroiditis, and thyroid cancer, over or underproduction of the adrenal steroid hormones, adrenogenital syndrome, Cushing's syndrome, Addison's disease of the adrenal cortex, Addison's pernicious anemia, primary and secondary aldosteronism, diabetes insipidus, diabetes mellitus, carcinoid syndrome, disturbances caused by the dysfunction of the parathyroid glands, pancreatic islet cell dysfunction, diabetes, disturbances of the endocrine system of the female like estrogen deficiency, resistant ovary syndrome; muscle weakness, myotonia. Duchenne's and other muscular dystrophies, dystrophia myotonica of Steinert, mitochondrial myopathies like disturbances of the catabolic metabolism in the muscle, carbohydrate and lipid storage myopathies, glycogenoses, myoglobinuria, malignant hyperthermia, polymyalgia rheumatics, dermatomyositis, multiple myositis, primary myocardial disease, cardiomyopathy; disorders of the ectoderm, neurofibromatosis, scleroderma and polyarteritis, Louis-Bar syndrome, von Hippel-Lindau disease, Sturge-Weber syndrome, tuberous sclerosis, amyloidosis, porphyria; sexual dysfunction of the male and female; confused states and seizures due to inappropriate secretion of antidiuretic hormone from the pituitary gland, Liddle's syndrome, Bartter's syndrome, Fanconi's I syndrome, or renal electrolyte wasting.

The disease, disorder, or condition may be or include a transplant rejection related condition, such as acute and chronic allograft rejection following solid organ transplant, for example, transplantation of kidney, heart, liver, lung, and cornea, chronic graft versus host disease, skin graft rejection, and bone marrow transplant rejection, or immunosuppression.

The disease, disorder, or condition may be or include a genitourinary related condition, such as nephritis (interstitial, acute interstitial (allergic), and glomerulonephritis), nephrotic syndrome, cystitis including acute and chronic (interstitial) cystitis and Hunner's ulcer, acute and chronic urethritis, prostatitis, epididymitis, oophoritis, salpingitis, vulvo vaginitis, vulvovaginal candidiasis, Peyronie's disease, and erectile dysfunction, renal disease, renal fibrosis, nephropyelitis, secondary contracted kidney, steroid dependent and steroid-resistant nephrosis, or Goodpasture's syndrome.

The disease, disorder, or condition may be or include a CNS related disease or disorder, such as neurodegenerative diseases, Alzheimer's disease and other cementing disorders including CJD and nvCJD, amyloidosis, and other demyelinating syndromes, cerebral atherosclerosis and vasculitis, temporal arteritis, myasthenia gravis, acute and chronic so pain (acute, intermittent or persistent, whether of central or peripheral origin) including post-operative, visceral pain, headache, migraine, neuralgia (including trigeminal), atypical facial pain, joint and bone pain, pain arising from cancer and tumor invasion, neuropathic pain syndromes including diabetic, post-herpetic, and HIV-associated neuropathies, neurosarcoidosis, to brain injuries, cerebrovascular diseases and their consequences, Parkinson's disease, corticobasal degeneration, motor neuron disease, dementia, including ALS (Amyotrophic-lateral sclerosis), multiple sclerosis, traumatic brain injury, stroke, post-stroke, post-traumatic brain injury, and small-vessel cerebrovascular disease, dementias, vascular dementia, dementia with Lewy bodies, frontotemporal dementia and Parkinsonism linked 1 to chromosome 17, frontotemporal dementias, including Pick's disease, progressive supranuclear palsy, corticobasal degeneration, Huntington's disease, thalamic degeneration, HIV dementia, schizophrenia with dementia, and Korsakoffs psychosis, within the meaning of the definition are also considered to be CNS disorders central and peripheral nervous system complications of malignant, infectious or autoimmune processes, algesia, cerebral infarction, attack, cerebral ischemia, head injury, spinal cord injury, myelopathic muscular atrophy, Shy-Drager syndrome, Reye's syndrome, progressive multifocal leukoencephalopathy, normal pressure hydrocephalus, sclerosing panencephalitis, frontal lobe type dementia, acute anterior poliomyelitis (poliomyelitis), poliomyelitis neurosis, viral encephalitis, allergic encephalomyelitis, epileptic encephalopathies, Creutzfeldt-Jakob disease, Kuru disease, bovine spongiform encephalopathy (mad cow disease), scrapie, epilepsy, cerebral amyloid angiopathy, depression, mania, manic-depressive psychosis, hereditary cerebellar ataxia, peripheral neuropathy, Nasu-Hakola syndrome, or Machado-Joseph disease.

The disease, disorder, or condition may be or include an inflammatory or immunological disease or disorder, such as general inflammation (of the ocular, nasal, pulmonary, and gastrointestinal passages), mastocytosis/mast cell disorders (cutaneous, systemic, mast cell activation syndrome, and pediatric mast cell diseases), mastitis (mammary gland), vaginitis, vasculitis (e.g., necrotizing, cutaneous, and hypersensitivity vasculitis), Wegener granulamatosis, myositis (including polymyositis, dermatomyositis), basophil related diseases including basophilic leukemia and basophilic leukocytosis, and eosinophil related diseases such as Churg-Strauss syndrome, eosinophilic granuloma, lupus erythematosus (such as, systemic lupus erythematosus, subacute cutaneous lupus erythematosus, and discoid lupus erythematosus), chronic thyroiditis, Hashimoto's thyroiditis, Grave's disease, type I diabetes, complications arising from diabetes mellitus, other immune disorders, eosinophilia fasciitis, hyper IgE syndrome, Addison's disease, antiphospholipid syndrome, immunodeficiency disease, acquired immune deficiency syndrome (AIDS), leprosy, Sezary syndrome, paraneoplastic syndromes, and other autoimmune disorders, fervescence, myositis, nervous diseases selected from multiple myositis, bursitis, Evans syndrome, leukotriene B4-mediated diseases, idiopathic hypoparathyroidism, nephrotic syndrome lupus, or immunosuppression.

The disease, disorder, or condition may be or include a cardiovascular disease or disorder, such as congestive heart failure, myocardial infarction, ischemic diseases of the heart, all kinds of atrial and ventricular arrhythmias, hypertension, cerebral trauma, occlusive vascular disease, stroke, cerebrovascular disorder, atherosclerosis, restenosis, affecting the coronary and peripheral is circulation, pericarditis, myocarditis, inflammatory and auto-immune cardiomyopathies including myocardial sarcoid, endocarditis, valvulitis, and aortitis including infective (e.g. syphilitic), hypertensive vascular diseases, peripheral vascular diseases, and atherosclerosis, vasculitides, disorders of the proximal and peripheral veins including phlebitis and thrombosis, including deep vein thrombosis and complications of varicose veins, aortic aneurism, periarteritis nodosa, cardiac fibrosis, post-myocardial infarction, idiopathic cardiomyopathy, or angioplasty.

The disease, disorder, or condition may be or include an oncological disease or disorder, such as common cancers (prostate, breast, lung, ovarian, pancreatic, bowel and colon, abdomen, stomach (and any other digestive system cancers), liver, pancreas, peritoneum, endocrine glands (adrenal, parathyroid, pituitary, testicles, ovary, thymus, thyroid), eye, head, neck, nervous system (central and peripheral), lymphatic system, blood, pelvic, skin, bone, soft tissue, spleen, thoracic, urogenital, and brain tumors), breast cancer, genitourinary cancer, lung cancer, gastrointestinal cancer, epidermoid cancer, melanoma, ovarian cancer, pancreas cancer, neuroblastoma, malignancies affecting the bone marrow (including the leukaemias) and lymphoproliferative systems, such as Hodgkin's and non-Hodgkin's lymphoma, B-cell lymphoma, follicular lymphoma, metastatic disease and tumor recurrences, and paraneoplastic syndromes, as well as hypergammaglobulinemia, lymphoproliferative diseases, disorders, and/or conditions, paraproteinemias, purpura (including idiopathic thrombocytopenic purpura), Waldenstron's Macroglobulinemia, Gaucher's Disease, histiocytosis, retinoblastoma and any other hyperproliferative disease, sarcomata, cachexia, tumor growth, tumor invasion, metastasis, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes e.g. acute or chronic myeloid leukaemia, or keratoleukoma.

The disease, disorder, or condition may be or include another disease or disorder, such as pain, migraine, sleep disorders, fever, sepsis, idiopathic thrombocytopenia pupura, post-operative adhesions, flushing, ischemic/reperfusion injury in the heart, brain, peripheral limbs, bacterial infection, viral infection, fungal infection, thrombosis, endotoxin shock, septic shock, thermal regulation including fever, Raynaud's disease, gangrene, diseases requiring anti-coagulation therapy, congestive heart failure, mucus secretion disorders, pulmonary hypotension, prostanoid-induced smooth muscle contract associated with dysmenorrhea and premature labor, premature delivery, reperfusion injury, burn, thermal injury, hemorrhage or traumatic shock, menstrual pain, menstrual cramp, dysmenorrhea, periodontosis, rickettsial infectious disease, protozoal disease, reproduction disease, toothache, pain after tooth extraction, Herpes zoster, Herpes simplex, retroperitoneal fibrosis, or various radiation injuries.

In certain embodiments, the disease is selected from the group consisting of an inflammatory disease, an autoimmune disease, an allergic disorder, and an ocular disorder. In certain embodiments, the disease is selected from the group consisting of pruritus, eczema, asthma, rhinitis, dry eye, ocular inflammation, allergic conjunctivitis, vernal conjunctivitis, vernal keratoconjunctivitis, giant papillary conjunctivitis, fungal keratitis and uveitis.

The method may include modulating the activity of one or more kinases in a subject, such as any of the kinase described above. The method may include inhibiting a kinase. The method may include activating, e.g., stimulating or enhancing the activity of, a kinase. The method may include modulating activity of a single kinase or preferentially modulating activity of a specific kinase over others. The method may include modulating activity of multiple kinases or preferentially modulating activity of two more specific kinases over others.

The method may include providing a compound of the invention. The method may include providing multiple compounds of the invention.

The method may include contacting cells containing a kinase with one or more compounds of the invention. For example and without limitation, contacting a cell with a compound may include exposing a cell to a compound, e.g., in a formulation, such as any of those described above; delivering a compound inside a cell; providing a compound to a subject and allowing a cell in the subject to become exposed to the compound. Contacting may be performed in vivo or in vitro. In vitro contact may include exposure of cells or tissue isolated from a subject. The method may include contacting cells with a single compound of the invention. The method may include contact cells with multiple compounds of the invention.

The method may include administration of a composition to a subject. The compositions may be provided by any suitable route of administration. For example and without limitation, the compositions may be administered buccally, by injection, dermally, enterally, intraarterially, intravenously, intranasally, e.g., by inhalation, intraocularly, orally, parenterally, pulmonarily, rectally, subcutaneously, systemically, topically, e.g., to the skin or eye, transdermally, or with or on an implantable medical device (e.g., stent or drug-eluting stent or balloon equivalents).

The method may include using a composition of the invention to diagnose a disease, disorder, or condition in a subject. For example, a radiolabeled form of a compound may be used a tracer in positron emission tomography (PET) to identify anatomical locations of aberrant kinase activity. PET is known in the art and described in, for example, Wadsak Wolfgang, Mitterhauser Markus (2010), “Basics and principles of radiopharmaceuticals for PET/CT”, European Journal of Radiology, 73 (3): 461-469. doi:10.1016/j.ejrad.2009.12.022, Bailey, D. L; D. W. Townsend; P E. Valk; M. N. Maisey (2005), Positron Emission Tomography: Basic Sciences. Secaucus, NJ: Springer-Verlag, ISBN 1-85233-798-2; and Carlson, Neil (Jan. 22, 2012). Physiology of Behavior. Methods and Strategies of Research, 11th edition, Pearson, p. 151, ISBN 0205239390, the contents of each of which are incorporated herein by reference. The invention may include administering one or more compositions of the invention for both diagnostic and therapeutic purposes.

### EXAMPLES

### General Synthetic Scheme

Compounds of this disclosure can be made by the methods depicted in the reaction schemes shown below. The starting materials and reagents used in preparing these compounds are either available from commercial suppliers such as Sigma-Aldrich Chemical Co., (Milwaukee, Wis.), Acros Organics, Bachem (Torrance, Calif.), Oakwood Chemicals, Matrix Chemicals, or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). Generic schemes 1 and 2 are merely illustrative of some methods by which the compounds of this disclosure, and pharmaceutically acceptable salts thereof, can be synthesized, and various modifications to these schemes can be made and will be suggested to one skilled in the art reading this disclosure. The starting materials, the intermediates, and the final products of the reaction(s) may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 200° C., such as from about 0° C. to about 125° C. and further such as at about room (or ambient) temperature, e.g., about 20° C. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

Compounds of this invention can be synthesized by the methods depicted in the general reaction Schemes shown below.

The following Schemes, i.e., Schemes 1 and 2, represent general synthetic methods for some of the compounds in this invention, i.e., compounds of formula (A) (B) (C) (D) (E) (F) (G) (H) (I), wherein R₁, R₂, X, Y, Z₁, Z₂, Z₃, W₁, W₂, V, L are the appropriate choices of, or appropriate chemical precursors for, the corresponding functional groups as defined in Formula (I) and (II) above.

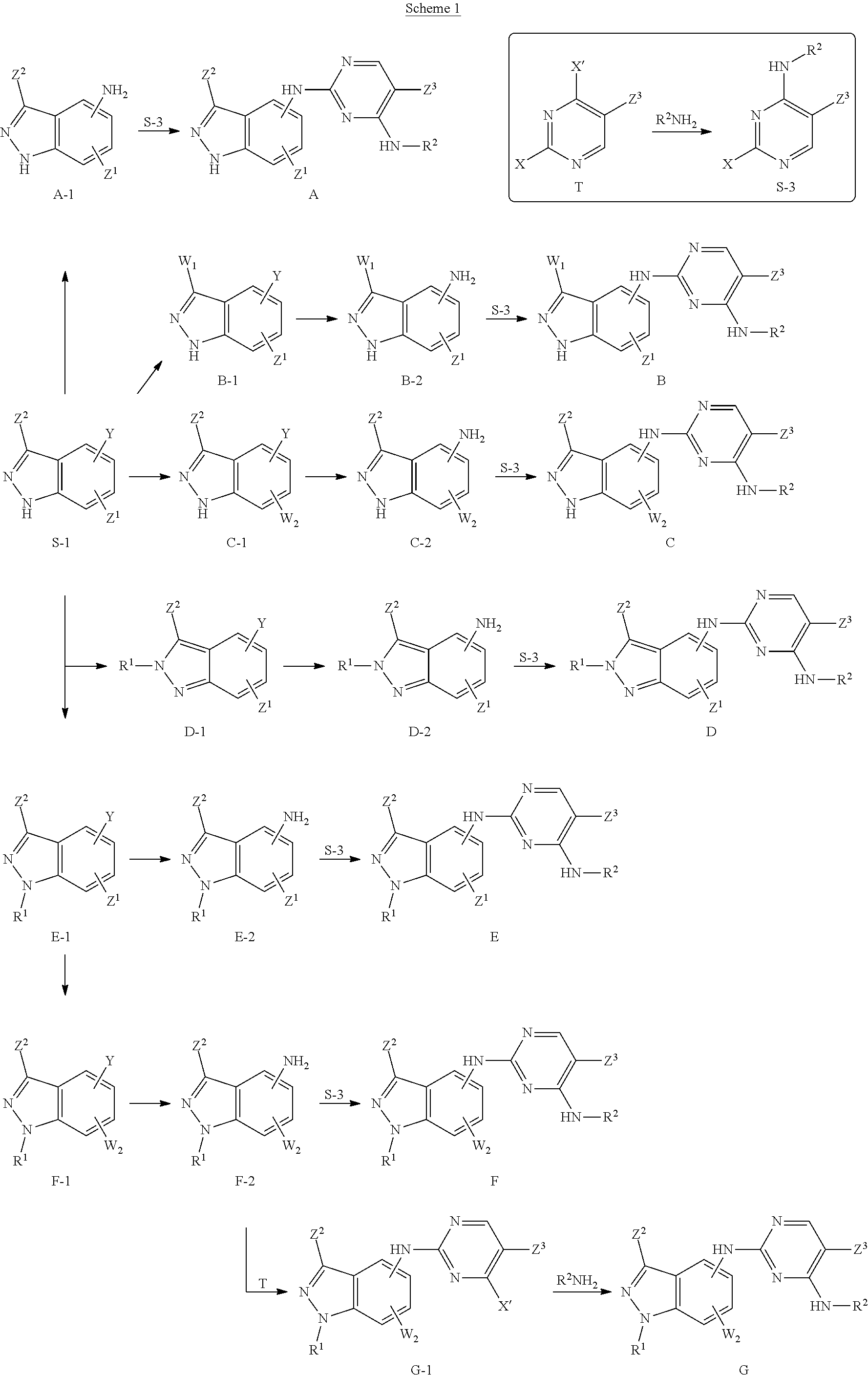

In Scheme 1, starting material T (X, X′=halo) was either purchased from commercial sources or prepared according to known literature methods. SnAr displacement reaction of compounds T with appropriate R²NH₂ led to the formation of intermediates S-3. Starting material S-1 (Y=halo or NO₂) was either purchased from commercial sources or prepared according to known literature methods. Intermediates A-1 were prepared from S-1 through coupling reaction (for example, Buchwald-Hartwig coupling) followed by the N-deprotection, or through nitro group reduction. Reacting A-1 with S-3 under appropriate displacement reaction conditions resulted in formation of compounds of formula A. Intermediates B-1 were made from S-1 through conventional functional group transformations (e.g. coupling reaction such as Buchwald-Hartwig and Heck reaction, amide formation and etc.). B-2 could be accessed from B-1 through Buchwald-Hartwig coupling reaction and then N-deprotection when —Y or —Z₁=halo or through direct nitro group reduction when —Y or —Z₁=—NO₂. Similarly, SnAr reaction between B-2 and S-3 led to the formation of compounds of formula B. Compounds of formula C were synthesized through the similar strategy described above for compounds of formula B, except that in the synthesis of intermediates C-1, the functional group transformation from Z¹ to W² were realized through either a coupling reaction (for example, Buchwald-Hartwig coupling and Heck reaction) or amide formation. Compounds D-1 and E-1 featuring N-substituted pyrazoles were synthesized from S-1 through direct alkylation, acylation or sulfonylation. The resulting products were subjected to further transformations (for example, Buchwald-Hartwig coupling followed by N-deprotection, or nitro group reduction) to provide intermediates D-2 and E-2 respectively. Reacting D-2 or E-2 with S-3 under SnAr condition completed the synthesis of compounds of formula D or E respectively. Intermediates F-1 were prepared from E-1 via coupling reactions with appropriate chemical counterparts (e.g. Buchwald-Hartwig reaction, Fleck reaction and etc.). Subsequent transformations from —Y to —NH₂ group led to the formation of intermediates F-2, which reacted with intermediate S-3 to provide compounds of formula F. SnAr displacement reaction between intermediates F-2 and intermediate T generated intermediates G-1, which were converted to compounds of formula G through the replacement of X′ by various R²NH₂.

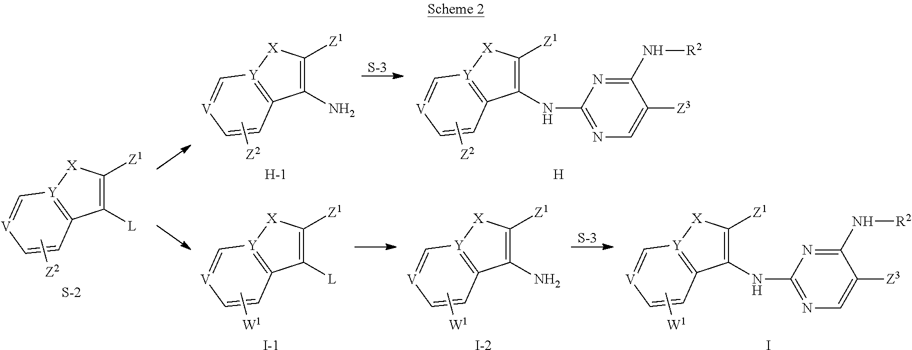

In Scheme 2, starting material S-2 (L=halo) was either purchased from commercial sources or prepared according to known literature methods. Functional group Y was converted to amino (NH₂) group either through coupling reaction with appropriate reacting partners (when L=halo, for example, Buchwald-Hartwig coupling), followed with deprotection as needed, or, via direct reduction of nitro group in S-2 (when L=NO₂). The resulting amino intermediate H-1 was further reacted with S-3 to provide compounds of formula H. Following same synthetic strategy, compounds of formula I was also made from S-2, except that an additional step was involved for indazole ring substituent transformation (from Z₂ in S-2 to W1 in I-1) via global reactions (for example, Buchwald-Hartwig coupling).

Representative compounds of Formula (A, B, C, D, E, F, G, H and I), or salts thereof, are disclosed in Table 1. Although Table 1 may show a specific salt of a compound of Formula (A, B, C, D, E, F, G, H and I), those skilled in the art will be able to recognize the parent compounds (wherein the “parent compound” is a compound without a salt moiety present), and other salts, such as pharmaceutically acceptable salts, of those compounds enumerated in Table 1

TABLE 1

| | | | MS |
|---|---|---|---|
| Ex. | | | Found |
| No. | Structure | Name | [M + H] |
| 1 | 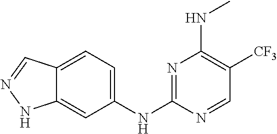 [SMILES: CNc1nc(Nc2ccc3cn[nH]c3c2)ncc1C(F)(F)F] | N2-(1H-indazol-6-yl)-N4-methyl- 5-(trifluoromethyl)pyrimidine-2,4- diamine | 309.1 |
| 2 | 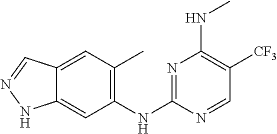 [SMILES: CNc1nc(Nc2cc3[nH]ncc3cc2C)ncc1C(F)(F)F] | N2-(1H-indazol-6-yl)-N4-methyl- 5-(trifluoromethyl)pyrimidine-2,4- diamine | 323.1 |
| 3 | 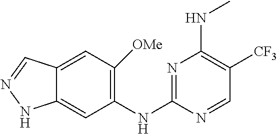 [SMILES: CNc1nc(Nc2cc3[nH]ncc3cc2OC)ncc1C(F)(F)F] | N2-(5-methoxy-1H-indazol-6-yl)- N4-methyl-5- (trifluoromethyl)pyrimidine-2,4- diamine | 339.1 |
| 4 | 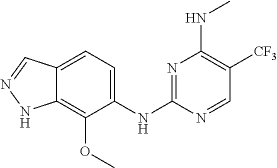 [SMILES: CNc1nc(Nc2ccc3cn[nH]c3c2OC)ncc1C(F)(F)F] | N2-(7-methoxy-1H-indazol-6-yl)- N4-methyl-5- (trifluoromethyl)pyrimidine-2,4- diamine | 339.0 |
| 5 | 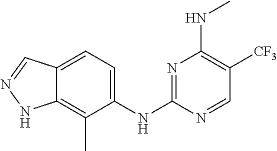 [SMILES: CNc1nc(Nc2ccc3cn[nH]c3c2C)ncc1C(F)(F)F] | N4-methyl-N2-(7-methyl-1H- indazol-6-yl)-5- (trifluoromethyl)pyrimidine-2,4- diamine | 323.1 |
| 6 | 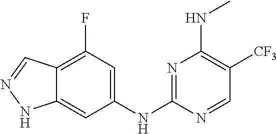 [SMILES: CNc1nc(Nc2cc(F)c3cn[nH]c3c2)ncc1C(F)(F)F] | N2-(4-fluoro-1H-indazol-6-yl)-N4- methyl-5- (trifluoromethyl)pyrimidine-2,4- diamine | 327.0 |
| 7 | 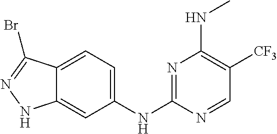 [SMILES: CNc1nc(Nc2ccc3c(Br)n[nH]c3c2)ncc1C(F)(F)F] | N2-(3-bromo-1H-indazol-6-yl)-N4- methyl-5- (trifluoromethyl)pyrimidine-2,4- diamine | 389.0 |
| 8 | 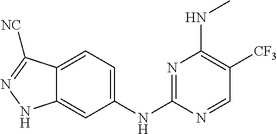 [SMILES: CNc1nc(Nc2ccc3c(C#N)n[nH]c3c2)ncc1C(F)(F)F] | 6-((4-(methylamino)-5- (trifluoromethyl)pyrimidin-2- yl)amino)-1H-indazole-3- carbonitrile | 334.0 |
| 9 | 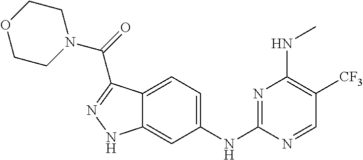 [SMILES: CNc1nc(Nc2ccc3c(C(=O)N4CCOCC4)n[nH]c3c2)ncc1C(F)(F)F] | (6-((4-(methylamino)-5- (trifluoromethyl)pyrimidin-2- yl)amino)-1H-indazol-3- yl)(morpholino)methanone | 422.1 |
| 10 | 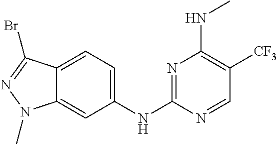 [SMILES: CNc1nc(Nc2ccc3c(Br)nn(C)c3c2)ncc1C(F)(F)F] | N²-(3-bromo-1-methyl-1H-indazol- 6-yl)-N⁴-methyl-5-(trifluoromethyl) pyrimidine-2,4-diamine | 403.0 |
| 11 | 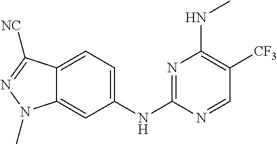 [SMILES: CNc1nc(Nc2ccc3c(C#N)nn(C)c3c2)ncc1C(F)(F)F] | 1-methyl-6-((4-(methylamino)-5- (trifluoromethyl)pyrimidin-2- yl)amino)-1H-indazole-3- carbonitrile | 348.0 |
| 12 | 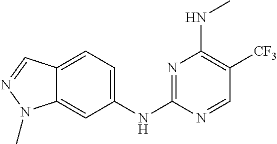 [SMILES: CNc1nc(Nc2ccc3cnn(C)c3c2)ncc1C(F)(F)F] | N4-methyl-N2-(1-methyl-1H- indazol-6-yl)-5- (trifluoromethyl)pyrimidine-2,4- diamine | 323.0 |
| 13 | 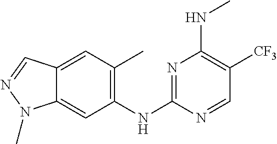 [SMILES: CNc1nc(Nc2cc3c(cnn3C)cc2C)ncc1C(F)(F)F] | N2-(1,5-dimethyl-1H-indazol-6-yl)- N4-methyl-5- (trifluoromethyl)pyrimidine-2,4- diamine | 337.1 |
| 14 | 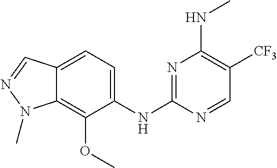 [SMILES: CNc1nc(Nc2ccc3cnn(C)c3c2OC)ncc1C(F)(F)F] | N2-(7-methoxy-1-methyl-1H- indazol-6-yl)-N4-methyl-5- (trifluoromethyl)pyrimidine-2,4- diamine | 353.3 |
| 15 | 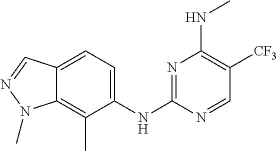 [SMILES: CNc1nc(Nc2ccc3cnn(C)c3c2C)ncc1C(F)(F)F] | N2-(1,7-dimethyl-1H-indazol-6-yl)- N4-methyl-S- (trifluoromethyl)pyrimidine-2,4- diamine | 337.1 |
| 16 | 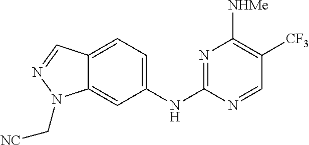 [SMILES: CNc1nc(Nc2ccc3cnn(CC#N)c3c2)ncc1C(F)(F)F] | 2-(6-((4-(methylamino)-5- (trifluoromethyl)pyrimidin-2- yl)amino)-1H-indazol-1- yl)acetonitrile | 348.1 |
| 17 | 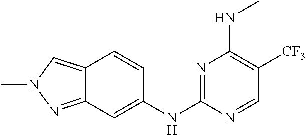 [SMILES: CNc1nc(Nc2ccc3cn(C)nc3c2)ncc1C(F)(F)F] | N4-methyl-N2-(2-methyl-2H- indazol-6-yl)-5- (trifluoromethyl)pyrimidine-2,4- diamine | 323.1 |
| 18 | 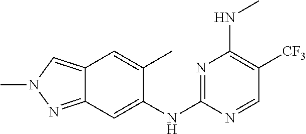 [SMILES: CNc1nc(Nc2cc3nn(C)cc3cc2C)ncc1C(F)(F)F] | N2-(2,5-dimethyl-2H-indazol-6-yl)- N4-methyl-5- (trifluoromethyl)pyrimidine-2,4- diamine | 337.1 |
| 19 | 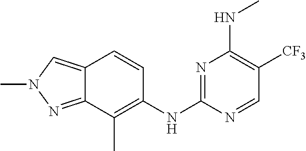 [SMILES: CNc1nc(Nc2ccc3cn(C)nc3c2C)ncc1C(F)(F)F] | N2-(2,7-dimethyl-2H-indazol-6-yl)- N4-methyl-5- (trifluoromethyl)pyrimidine-2,4- diamine | 337.1 |
| 20 | 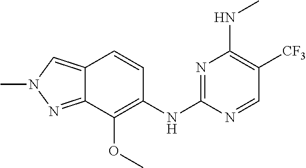 [SMILES: CNc1nc(Nc2ccc3cn(C)nc3c2OC)ncc1C(F)(F)F] | N2-(7-methoxy-2-methyl-2H- indazol-6-yl)-N4-methyl-5- (trifluoromethyl)pyrimidine-2,4- diamine | 353.3 |
| 21 | 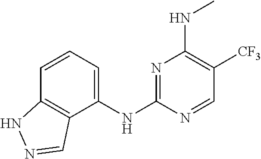 [SMILES: CNc1nc(Nc2cccc3[nH]ncc23)ncc1C(F)(F)F] | N2-(1H-indazol-4-yl)-N4-methyl- 5-(trifluoromethyl)pyrimidine-2,4- diamine | 309.0 |
| 22 | 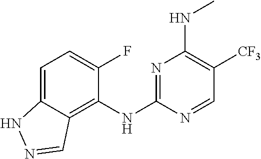 [SMILES: CNc1nc(Nc2c(F)ccc3[nH]ncc23)ncc1C(F)(F)F] | N2-(5-fluoro-1H-indazol-4-yl)-N4- methyl-5- (trifluoromethyl)pyrimidine-2,4- diamine | 327.0 |
| 23 | 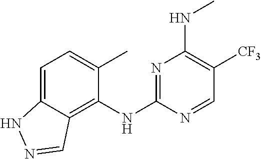 [SMILES: CNc1nc(Nc2c(C)ccc3[nH]ncc23)ncc1C(F)(F)F] | N4-methyl-N2-(5-methyl-1H- indazol-4-yl)-5- (trifluoromethyl)pyrimidine-2,4- diamine | 323.0 |
| 24 | 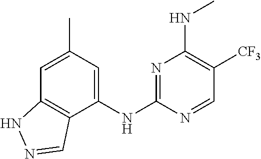 [SMILES: CNc1nc(Nc2cc(C)cc3[nH]ncc23)ncc1C(F)(F)F] | N4-methyl-N2-(6-methyl-1H- indazol-4-yl)-5- (trifluoromethyl)pyrimidine-2,4- diamine | 323.1 |
| 25 | 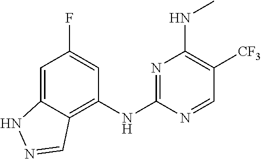 [SMILES: CNc1nc(Nc2cc(F)cc3[nH]ncc23)ncc1C(F)(F)F] | N2-(6-fluoro-1H-indazol-4-yl)-N4- methyl-5- (trifluoromethyl)pyrimidine-2,4- diamine | 327.1 |
| 26 | 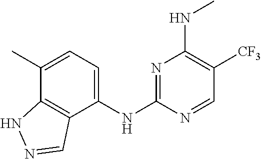 [SMILES: CNc1nc(Nc2ccc(C)c3[nH]ncc23)ncc1C(F)(F)F] | N4-methyl-N2-(7-methyl-1H- indazol-4-yl)-5- (trifluoromethyl)pyrimidine-2,4- diamine | 323.2 |
| 27 | 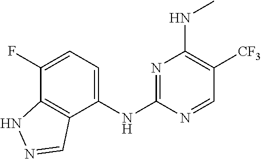 [SMILES: CNc1nc(Nc2ccc(F)c3[nH]ncc23)ncc1C(F)(F)F] | N2-(7-fluoro-1H-indazol-4-yl)-N4- methyl-5- (trifluoromethyl)pyrimidine-2,4- diamine | 323.1 |
| 28 | 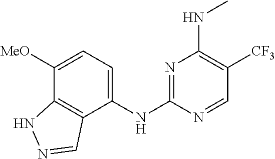 [SMILES: CNc1nc(Nc2ccc(OC)c3[nH]ncc23)ncc1C(F)(F)F] | N2-(7-methoxy-1H-indazol-4-yl)- N4-methyl-5- (trifluoromethyl)pyrimidine-2,4- diamine | 339.0 |
| 29 | 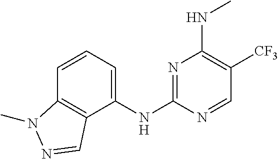 [SMILES: CNc1nc(Nc2cccc3c2cnn3C)ncc1C(F)(F)F] | N4-methyl-N2-(1-methyl-1H- indazol-4-yl)-5- (trifluoromethyl)pyrimidine-2,4- diamine | 323.1 |
| 30 | 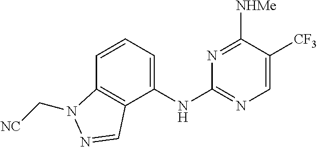 [SMILES: CNc1nc(Nc2cccc3c2cnn3CC#N)ncc1C(F)(F)F] | 2-(4-((4-(methylamino)-5- (trifluoromethyl)pyrimidin-2- yl)amino)-1H-indazol-1- yl)acetonitrile | 348.1 |
| 31 | 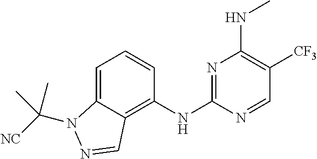 [SMILES: CNc1nc(Nc2cccc3c2cnn3C(C)(C)C#N)ncc1C(F)(F)F] | 2-methyl-2-(4-((4-(methylamino)- 5-(trifluoromethyl)pyrimidin-2- yl)amino)-1H-indazol-1- yl)propanenitrile | 376.1 |
| 32 | 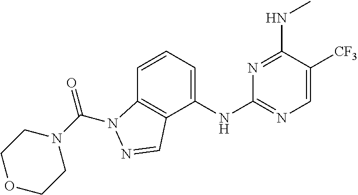 [SMILES: CNc1nc(Nc2cccc3c2cnn3C(=O)N2CCOCC2)ncc1C(F)(F)F] | (4-((4-(methylamino)-5- (trifluoromethyl)pyrimidin-2- yl)amino)-1H-indazol-1- yl)(morpholino)methanone | 422.1 |
| 33 | 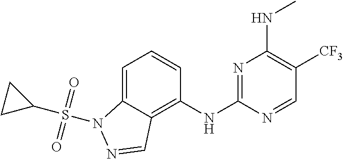 [SMILES: CNc1nc(Nc2cccc3c2cnn3S(=O)(=O)C2CC2)ncc1C(F)(F)F] | N2-(1-(cyclopropylsulfonyl)-1H- indazol-4-yl)-N4-methyl-5- (trifluoromethyl)pyrimidine-2,4- diamine | 413.1 |
| 34 | 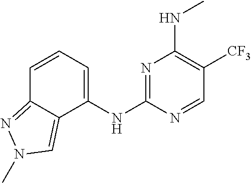 [SMILES: CNc1nc(Nc2cccc3nn(C)cc23)ncc1C(F)(F)F] | N4-methyl-N2-(2-methyl-2H- indazol-4-yl)-5- (trifluoromethyl)pyrimidine-2,4- diamine | 323.2 |
| 35 | 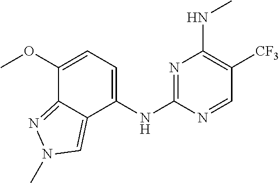 [SMILES: CNc1nc(Nc2ccc(OC)c3nn(C)cc23)ncc1C(F)(F)F] | N2-(7-methoxy-2-methyl-2H- indazol-4-yl)-N4-methyl-5- (trifluoromethyl)pyrimidine-2,4- diamine | 353.0 |
| 36 | 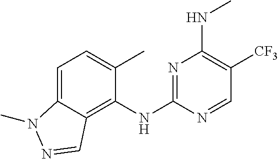 [SMILES: CNc1nc(Nc2c(C)ccc3c2cnn3C)ncc1C(F)(F)F] | N2-(1,5-dimethyl-1H-indazol-4-yl)- N4-methyl-5- (trifluoromethyl)pyrimidine-2,4- diamine | 337.2 |
| 37 | 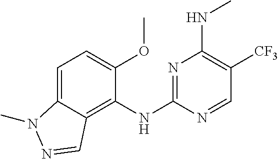 [SMILES: CNc1nc(Nc2c(OC)ccc3c2cnn3C)ncc1C(F)(F)F] | N2-(5-methoxy-1-methyl-1H- indazol-4-yl)-N4-methyl-5- (trifluoromethyl)pyrimidine-2,4- diamine | 353.2 |
| 38 | 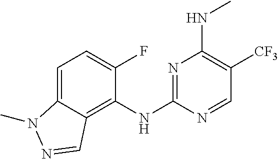 [SMILES: CNc1nc(Nc2c(F)ccc3c2cnn3C)ncc1C(F)(F)F] | N2-(5-fluoro-1-methyl-1H-indazol- 4-yl)-N4-methyl-5- (trifluoromethyl)pyrimidine-2,4- diamine | 341.1 |
| 39 | 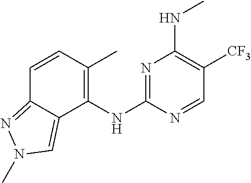 [SMILES: CNc1nc(Nc2c(C)ccc3nn(C)cc23)ncc1C(F)(F)F] | N2-(2,5-dimethyl-2H-indazol-4-yl)- N4-methyl-5- (trifluoromethyl)pyrimidine-2,4- diamine | 337.1 |
| 40 | 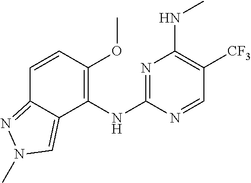 [SMILES: CNc1nc(Nc2c(OC)ccc3nn(C)cc23)ncc1C(F)(F)F] | N2-(5-methoxy-2-methyl-2H- indazol-4-yl)-N4-methyl-5- (trifluoromethyl)pyrimidine-2,4- diamine | 353.1 |
| 41 | 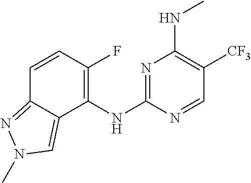 [SMILES: CNc1nc(Nc2c(F)ccc3nn(C)cc23)ncc1C(F)(F)F] | N2-(5-fluoro-2-methyl-2H-indazol- 4-yl)-N4-methyl-5- (trifluoromethyl)pyrimidine-2,4- diamine | 341.1 |
| 42 | 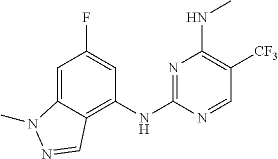 [SMILES: CNc1nc(Nc2cc(F)cc3c2cnn3C)ncc1C(F)(F)F] | N2-(6-fluoro-1-methyl-1H-indazol- 4-yl)-N4-methyl-5- (trifluoromethyl)pyrimidine-2,4- diamine | 341.1 |
| 43 | 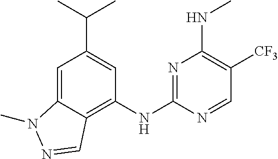 [SMILES: CNc1nc(Nc2cc(C(C)C)cc3c2cnn3C)ncc1C(F)(F)F] | N2-(6-isopropyl-1-methyl-1H- indazol-4-yl)-N4-methyl-5~ (trifluoromethyl)pyrimidine-2,4- diamine | 365.1 |
| 44 | 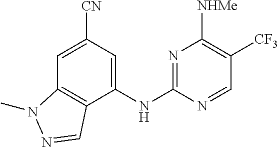 [SMILES: CNc1nc(Nc2cc(C#N)cc3c2cnn3C)ncc1C(F)(F)F] | 1-methyl-4-((4-(methylamino)-5- (trifluoromethyl)pyrimidin-2- yl)amino)-1H-indazole-6- carbonitrile | 348.2 |
| 45 | 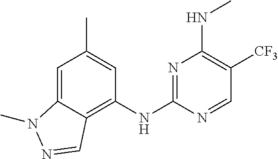 [SMILES: CNc1nc(Nc2cc(C)cc3c2cnn3C)ncc1C(F)(F)F] | N2-(1,6-dimethyl-1H-indazol-4-yl)- N4-methyl-5- (trifluoromethyl)pyrimidine-2,4- diamine | 337.1 |
| 46 | 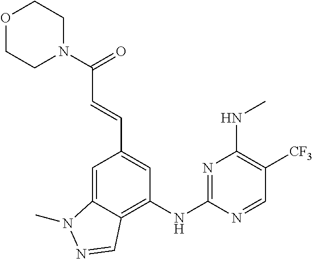 [SMILES: CNc1nc(Nc2cc(/C=C/C(=O)N3CCOCC3)cc3c2cnn3C)ncc1C(F)(F)F] | (E)-3-(1-methyl-4-((4- (methylamino)-5- (trifluoromethyl)pyrimidin-2- yl)amino)-1H-indazol-6-yl)-1- morpholinoprop-2-en-1-one | 462.2 |
| 47 | 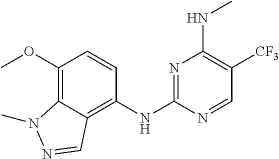 [SMILES: CNc1nc(Nc2ccc(OC)c3c2cnn3C)ncc1C(F)(F)F] | N2-(7-methoxy-1-methyl-1H- indazol-4-yl)-N4-methyl-5- (trifluoromethyl)pyrimidine-2,4- diamine | 353.1 |
| 48 | 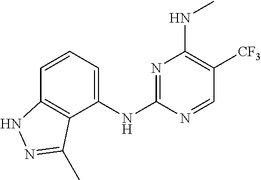 [SMILES: CNc1nc(Nc2cccc3[nH]nc(C)c23)ncc1C(F)(F)F] | N4-methyl-N2-(3-methyl-1H- indazol-4-yl)-5- (trifluoromethyl)pyrimidine-2,4- diamine | 323.1 |
| 49 | 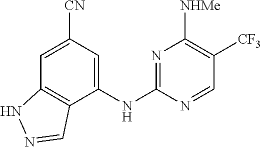 [SMILES: CNc1nc(Nc2cc(C#N)cc3[nH]ncc23)ncc1C(F)(F)F] | 4-((4-(methylamino)-5- (trifluoromethyl)pyrimidin-2- yl)amino)-1H-indazole-6- carbonitrile | 334.1 |
| 50 | 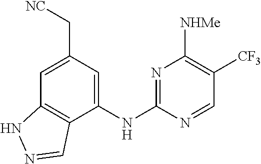 [SMILES: CNc1nc(Nc2cc(CC#N)cc3[nH]ncc23)ncc1C(F)(F)F] | 2-(4-((4-(methylamino)-5- (trifluoromethyl)pyrimidin-2- yl)amino)-1H-indazol-6- yl)acetonitrile | 348.1 |
| 51 | 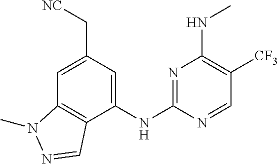 [SMILES: CNc1nc(Nc2cc(CC#N)cc3c2cnn3C)ncc1C(F)(F)F] | 2-(1-methyl-4-((4-(methylamino)- 5-(trifluoromethyl)pyrimidin-2- yl)amino)-1H-indazol-6- yl)acetonitrile | 362.2 |
| 52 | 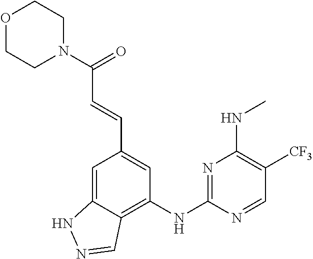 [SMILES: CNc1nc(Nc2cc(/C=C/C(=O)N3CCOCC3)cc3[nH]ncc23)ncc1C(F)(F)F] | (E)-3-(4-((4-(methylamino)-5- (trifluoromethyl)pyrimidin-2- yl)amino)-1H-indazol-6-yl)-1- morpholinoprop-2-en-1-one | 448.3 |
| 53 | 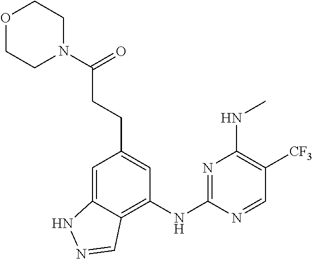 [SMILES: CNc1nc(Nc2cc(CCC(=O)N3CCOCC3)cc3[nH]ncc23)ncc1C(F)(F)F] | 3-(4-((4-(methylamino)-5- (trifluoromethyl)pyrimidin-2- yl)amino)-1H-indazol-6-yl)-1- morpholinopropan-1-one | 450.3 |
| 54 | 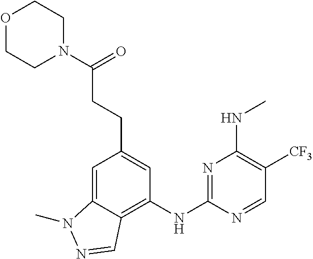 [SMILES: CNc1nc(Nc2cc(CCC(=O)N3CCOCC3)cc3c2cnn3C)ncc1C(F)(F)F] | 3-(1-methyl-4-((4-(methylamino)- 5-(trifluoromethyl)pyrimidin-2- yl)amino)-1H-indazol-6-yl)-1- morpholinopropan-1-one | 464.2 |
| 55 | 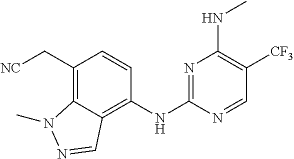 [SMILES: CNc1nc(Nc2ccc(CC#N)c3c2cnn3C)ncc1C(F)(F)F] | 2-(1-methyl-4-((4-(methylamino)- 5-(trifluoromethyl)pyrimidin-2- yl)amino)-1H-indazol-7- yl)acetonitrile | 362.2 |
| 56 | 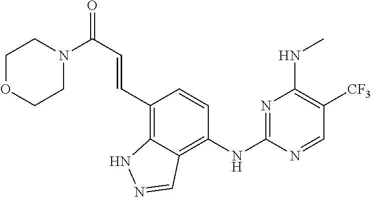 [SMILES: CNc1nc(Nc2ccc(/C=C/C(=O)N3CCOCC3)c3[nH]ncc23)ncc1C(F)(F)F] | (E)-3-(4-((4-(methylamino)-5- (trifluoromethyl)pyrimidin-2- yl)amino)-1H-indazol-7-yl)-1- morpholinoprop-2-en-1-one | 448.3 |
| 57 | 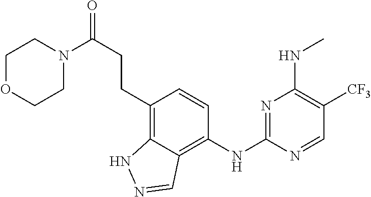 [SMILES: CNc1nc(Nc2ccc(CCC(=O)N3CCOCC3)c3[nH]ncc23)ncc1C(F)(F)F] | 3-(4-((4-(methylamino)-5- (trifluoromethyl)pyrimidin-2- yl)amino)-1H-indazol-7-yl)-1- morpholinopropan-1-one | 450.2 |
| 58 | 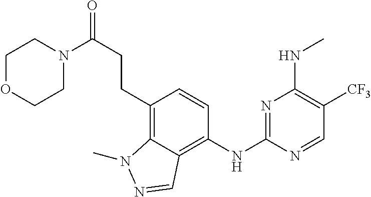 [SMILES: CNc1nc(Nc2ccc(CCC(=O)N3CCOCC3)c3c2cnn3C)ncc1C(F)(F)F] | 3-(1-methyl-4-((4-(methylamino)- 5-(trifluoromethyl)pyrimidin~2- yl)amino)-1H-indazol-7-yl)-1- morpholinopropan-1-one | 464.3 |
| 59 | 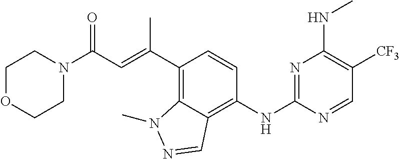 [SMILES: CNc1nc(Nc2ccc(/C(C)=C/C(=O)N3CCOCC3)c3c2cnn3C)ncc1C(F)(F)F] | (E)-3-(1-methyl-4-((4- (methylamino)-5- (trifluoromethyl)pyrimidin-2- yl)amino)-1H-indazol-7-yl)-1- morpholinobut-2-en-1-one | 476.4 |
| 60 | 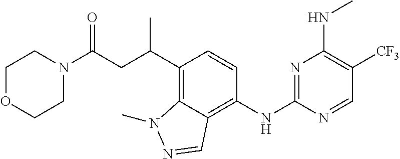 [SMILES: CNc1nc(Nc2ccc(C(C)CC(=O)N3CCOCC3)c3c2cnn3C)ncc1C(F)(F)F] | 3-(1-methyl-4-((4-(methylamino)- 5-(trifluoromethyl)pyrimidin-2- yl)amino)-1H-indazol-7-yl)-1- morpholinobutan-1-one | 478.4 |
| 61 | 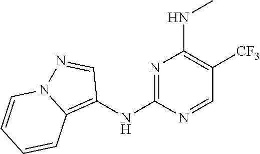 [SMILES: CNc1nc(Nc2cnn3ccccc23)ncc1C(F)(F)F] | N4-methyl-N2-(pyrazolo[1,5- a]pyridin-3-yl)-5- (trifluoromethyl)pyrimidine-2,4- diamine | 309.0 |
| 62 | 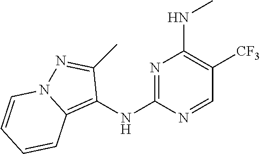 [SMILES: CNc1nc(Nc2c(C)nn3ccccc23)ncc1C(F)(F)F] | N4-methyl-N2-(2- methylpyrazolo[1,5-a]pyridin-3-yl)- S-(trifluoromethyl)pyrimidine-2,4- diamine | 323.0 |
| 63 | 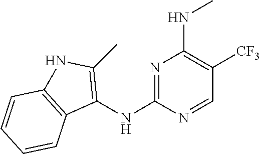 [SMILES: CNc1nc(Nc2c(C)[nH]c3ccccc23)ncc1C(F)(F)F] | N4-methyl-N2-(2-methyl-1H-indol- 3-yl)-5- (trifluoromethyl)pyrimidine-2,4- diamine | 322.1 |
| 64 | 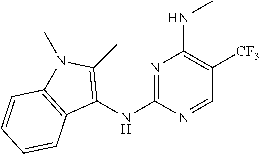 [SMILES: CNc1nc(Nc2c(C)n(C)c3ccccc23)ncc1C(F)(F)F] | N2-(1,2-dimethyl-1H-indol-3-yl)- N4-methyl-5- (trifluoromethyl)pyrimidine-2,4- diamine | 336.1 |
| 65 | 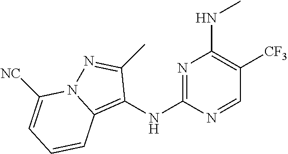 [SMILES: CNc1nc(Nc2c(C)nn3c(C#N)cccc23)ncc1C(F)(F)F] | 2-methyl-3-((4-(methylamino)-5- (trifluoromethyl)pyrimidin-2- yl)amino)pyrazolo[1,5-a]pyridine- 7-carbonitrile | 348.1 |
| 66 | 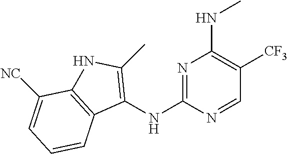 [SMILES: CNc1nc(Nc2c(C)[nH]c3c(C#N)cccc23)ncc1C(F)(F)F] | 2-methyl-3-((4-(methylamino)-5- (trifluoromethyl)pyrimidin-2- yl)amino)-1H-indole-7-carbonitrile | 347.1 |
| 67 | 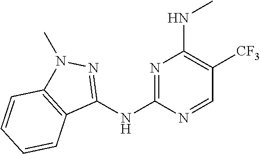 [SMILES: CNc1nc(Nc2nn(C)c3ccccc23)ncc1C(F)(F)F] | N4-methyl-N2-(1-methyl-1H- indazol-3-yl)-5- (trifluoromethyl)pyrimidine-2,4- diamine | 323.1 |
| 68 | 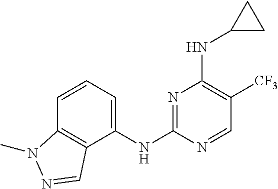 [SMILES: Cn1ncc2c(Nc3ncc(C(F)(F)F)c(NC4CC4)n3)cccc21] | N4-cyclopropyl-N2-(1-methyl-1H- indazol-4-yl)-5- (trifluoromethyl)pyrimidine-2,4- diamine | 349.1 |
| 69 | 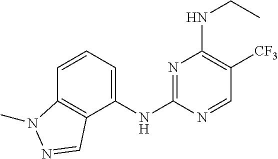 [SMILES: CCNc1nc(Nc2cccc3c2cnn3C)ncc1C(F)(F)F] | N4-ethyl-N2-(1-methyl-1H- indazol-4-yl)-5- (trifluoromethyl)pyrimidine-2,4- diamine | 337.1 |
| 70 | 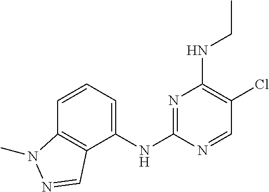 [SMILES: CCNc1nc(Nc2cccc3c2cnn3C)ncc1Cl] | 5-chloro-N4-ethyl-N2-(1-methyl- 1H-indazol-4-yl)pyrimidine-2,4- diamine | 303.1 |
| 71 | 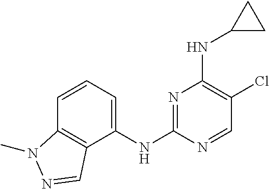 [SMILES: Cn1ncc2c(Nc3ncc(Cl)c(NC4CC4)n3)cccc21] | 5-chloro-N4-cyclopropyl-N2-(1- methyl-1H-indazol-4- yl)pyrimidine-2,4-diamine | 315.0 |
| 72 | 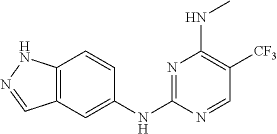 [SMILES: CNc1nc(Nc2ccc3[nH]ncc3c2)ncc1C(F)(F)F] | N2-(1H-indazol-5-yl)-N4-methyl- 5-(trifluoromethyl)pyrimidine-2,4- diamine | 309.1 |
| 73 | 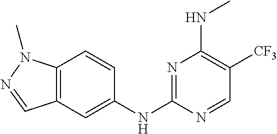 [SMILES: CNc1nc(Nc2ccc3c(cnn3C)c2)ncc1C(F)(F)F] | N4-methyl-N2-(1-methyl-1H- indazol-5-yl)-5- (trifluoromethyl)pyrimidine-2,4- diamine | 323.0 |
| 74 | 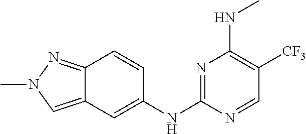 [SMILES: CNc1nc(Nc2ccc3nn(C)cc3c2)ncc1C(F)(F)F] | N4-methyl-N2-(2-methyl-2H- indazol-5-yl)-5- (trifluoromethyl)pyrimidine-2,4- diamine | 323.1 |
| 75 | 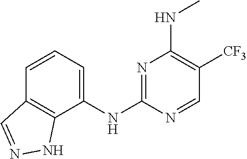 [SMILES: CNc1nc(Nc2cccc3cn[nH]c23)ncc1C(F)(F)F] | N2-(1H-indazol-7-yl)-N4-methyl- 5-(trifluoromethyl)pyrimidine-2,4- diamine | 309.0 |
| 76 | 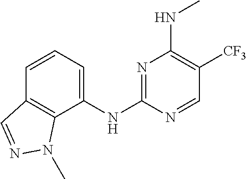 [SMILES: CNc1nc(Nc2cccc3cnn(C)c23)ncc1C(F)(F)F] | N4-methyl-N2-(1-methyl-1H- indazol-7-yl)-5- (trifluoromethyl)pyrimidine-2,4- diamine | 323.1 |
| 77 | 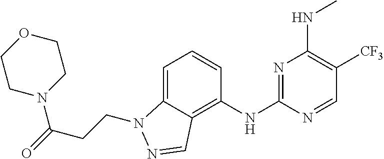 [SMILES: CNc1nc(Nc2cccc3c2cnn3CCC(=O)N2CCOCC2)ncc1C(F)(F)F] | 3-(4-((4-(methylamino)-5- (trifluoromethyl)pyrimidin-2- yl)amino)-1H-indazol-1-yl)-1- morpholinopropan-1-one | 450.2 |
| 78 | 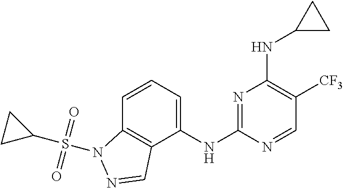 [SMILES: O=S(=O)(C1CC1)n1ncc2c(Nc3ncc(C(F)(F)F)c(NC4CC4)n3)cccc21] | N4-cyclopropyl-N2-(1- (cyclopropylsulfonyl)-1H-indazol- 4-yl)-5- (trifluoromethyl)pyrimidine-2,4- diamine | 439.1 |
| 79 | 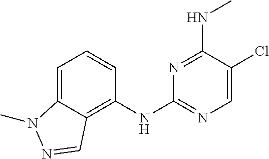 [SMILES: CNc1nc(Nc2cccc3c2cnn3C)ncc1Cl] | 5-chloro-N4-methyl-N2-(1-methyl- 1H-indazol-4-yl)pyrimidine-2,4- diamine | 289.0 |
| 80 | 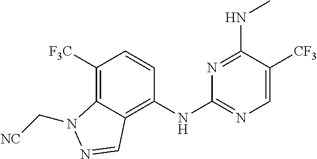 [SMILES: CNc1nc(Nc2ccc(C(F)(F)F)c3c2cnn3CC#N)ncc1C(F)(F)F] | 2-(4-((4-(methylamino)-5- (trifluoromethyl)pyrimidin-2- yl)amino)-7-(trifluoromethyl)-1H- indazol-1-yl)acetonitrile | 416.2 |
| 81 | 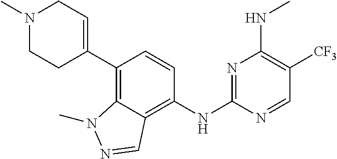 [SMILES: CNc1nc(Nc2ccc(C3=CCN(C)CC3)c3c2cnn3C)ncc1C(F)(F)F] | N4-methyl-N2-(1-methyl-7-(1- methyl-1,2,3,6-tetrahydropyridin-4- yl)-1H-indazol-4-yl)-5- (trifluoromethyl)pyrimidine-2,4- diamine | 418.3 |
| 82 | 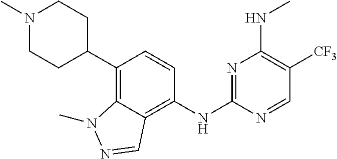 [SMILES: CNc1nc(Nc2ccc(C3CCN(C)CC3)c3c2cnn3C)ncc1C(F)(F)F] | N4-methyl-N2-(1-methyl-7-(1- methylpiperidin-4-yl)-1H-indazol- 4-yl)-5- (trifluoromethyl)pyrimidine-2,4- diamine | 420.3 |
| 83 | 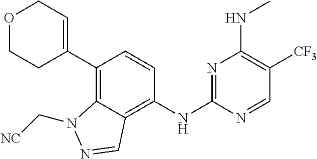 [SMILES: CNc1nc(Nc2ccc(C3=CCOCC3)c3c2cnn3CC#N)ncc1C(F)(F)F] | 2-(7-(3,6-dihydro-2H-pyran-4-yl)- 4-((4-(methylamino)-5- (trifluoromethyl)pyrimidin-2- yl)amino)-1H-indazol-1- yl)acetonitrile | 430.2 |
| 84 | 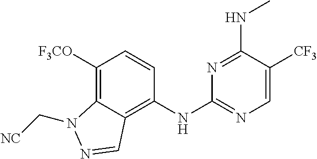 [SMILES: CNc1nc(Nc2ccc(OC(F)(F)F)c3c2cnn3CC#N)ncc1C(F)(F)F] | 2-(4-((4-(methylamino)-5- (trifluoromethyl)pyrimidin-2- yl)amino)-7-(trifluoromethoxy)- 1H-indazol-1-yl)acetonitrile | 432.2 |
| 85 | 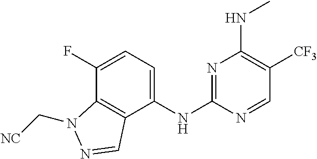 [SMILES: CNc1nc(Nc2ccc(F)c3c2cnn3CC#N)ncc1C(F)(F)F] | 2-(7-fluoro-4-((4-(methylamino)-5- (trifluoromethyl)pyrimidin-2- yl)amino)-1H-indazol-1- yl)acetonitrile | 366.1 |
| 86 | 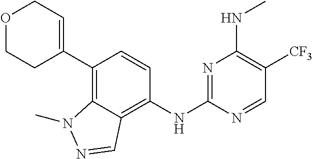 [SMILES: CNc1nc(Nc2ccc(C3=CCOCC3)c3c2cnn3C)ncc1C(F)(F)F] | N2-(7-(3,6-dihydro-2H-pyran-4- yl)-1-methyl-1H-indazol-4-yl)-N4- methyl-5- (trifluoromethyl)pyrimidine-2,4- diamine | 405.1 |
| 87 | 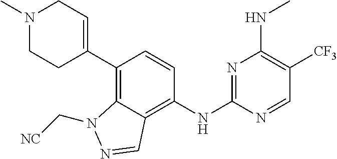 [SMILES: CNc1nc(Nc2ccc(C3=CCN(C)CC3)c3c2cnn3CC#N)ncc1C(F)(F)F] | 2-(7-(1-methyl-1,2,3,6- tetrahydropyridin-4-yl)-4-((4- (methylamino)-5- (trifluoromethyl)pyrimidin-2- yl)amino)-1H-indazol-1- yl)acetonitrile | 443.3 |
| 88 | 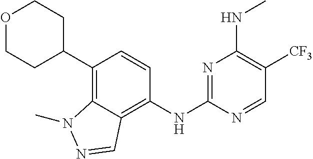 [SMILES: CNc1nc(Nc2ccc(C3CCOCC3)c3c2cnn3C)ncc1C(F)(F)F] | N4-methyl-N2-(1-methyl-7- (tetrahydro-2H-pyran-4-yl)-1H- indazol-4-yl)-5- (trifluoromethyl)pyrimidine-2,4- diamine | 407.3 |
| 89 | 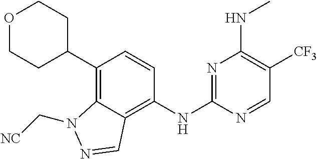 [SMILES: CNc1nc(Nc2ccc(C3CCOCC3)c3c2cnn3CC#N)ncc1C(F)(F)F] | 2-(4-((4-(methylamino)-5- (trifluoromethyl)pyrimidin-2- yl)amino)-7-(tetrahydro-2H-pyran- 4-yl)-1H-indazol-1-yl)acetonitrile | 432.2 |
| 90 | 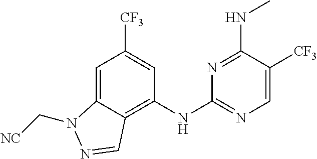 [SMILES: CNc1nc(Nc2cc(C(F)(F)F)cc3c2cnn3CC#N)ncc1C(F)(F)F] | 2-(4-((4-(methylamino)-5- (trifluoromethyl)pyrimidin-2- yl)amino)-6-(trifluoromethyl)-1H- indazol-1-yl)acetonitrile | 416.1 |
| 91 | 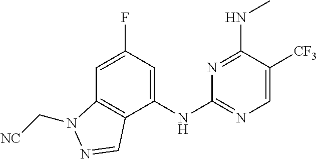 [SMILES: CNc1nc(Nc2cc(F)cc3c2cnn3CC#N)ncc1C(F)(F)F] | 2-(6-fluoro-4-((4-(methylamino)-5- (trifluoromethyl)pyrimidin-2- yl)amino)-1H-indazol-1- yl)acetonitrile | 366.1 |
| 92 | 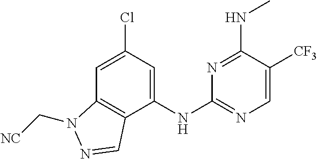 [SMILES: CNc1nc(Nc2cc(Cl)cc3c2cnn3CC#N)ncc1C(F)(F)F] | 2-(6-chloro-4-((4-(methylamino)-5- (trifluoromethyl)pyrimidin-2- yl)amino)-1H-indazol-1- yl)acetonitrile | 382.2 |
| 93 | 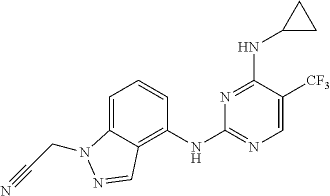 [SMILES: N#CCn1ncc2c(Nc3ncc(C(F)(F)F)c(NC4CC4)n3)cccc21] | 2-(4-((4-(cyclopropylamino)-5- (trifluoromethyl)pyrimidin-2- yl)amino)-1H-indazol-1- yl)acetonitrile | 374.1 |
| 94 | 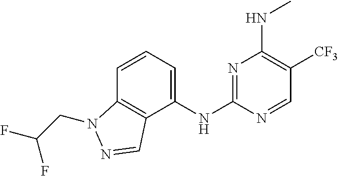 [SMILES: CNc1nc(Nc2cccc3c2cnn3CC(F)F)ncc1C(F)(F)F] | N2-(1-(2,2-difluoroethyl)-1H- indazol-4-yl)-N4-methyl-5- (trifluoromethyl)pyrimidine-2,4- diamine | 373.1 |
| 95 | 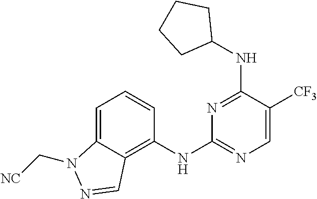 [SMILES: N#CCn1ncc2c(Nc3ncc(C(F)(F)F)c(NC4CCCC4)n3)cccc21] | 2-(4-((4-(cyclopentylamino)-5- (trifluoromethyl)pyrimidin-2- yl)amino)-1H-indazol-1- yl)acetonitrile | 402.1 |
| 96 | 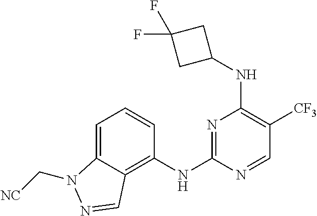 [SMILES: N#CCn1ncc2c(Nc3ncc(C(F)(F)F)c(NC4CC(F)(F)C4)n3)cccc21] | 2-(4-((4-((3,3- difluorocyclobutyl)amino)-5- (trifluoromethyl)pyrimidin-2- yl)amino)-1H-indazol-1- yl)acetonitrile | 424.1 |
| 97 | 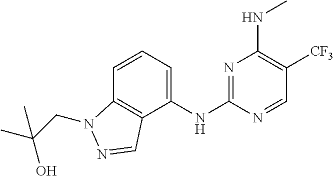 [SMILES: CNc1nc(Nc2cccc3c2cnn3CC(C)(C)O)ncc1C(F)(F)F] | 2-methyl-1-(4-((4-(methylamino)- 5-(trifluoromethyl)pyrimidin-2- yl)amino)-1H-indazol-1-yl)propan- 2-ol | 381.3 |
| 98 | 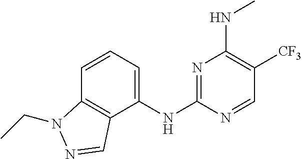 [SMILES: CCn1ncc2c(Nc3ncc(C(F)(F)F)c(NC)n3)cccc21] | N2-(1-ethyl-1H-indazol-4-yl)-N4- methyl-5- (trifluoromethyl)pyrimidine-2,4 diamine | 337.2 |
| 99 | 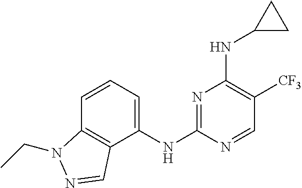 [SMILES: CCn1ncc2c(Nc3ncc(C(F)(F)F)c(NC4CC4)n3)cccc21] | N4-cyclopropyl-N2-(1-ethyl-1H- indazol-4-yl)-5- (trifluoromethyl)pyrimidine-2,4- diamine | 363.1 |
| 100 | 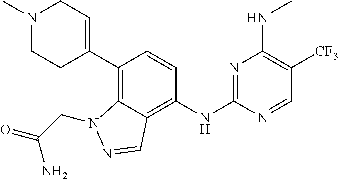 [SMILES: CNc1nc(Nc2ccc(C3=CCN(C)CC3)c3c2cnn3CC(N)=O)ncc1C(F)(F)F] | 2-(7-(1-methyl-1,2,3,6- tetrahydropyridin-4-yl)-4-((4- (methylamino)-5- (trifluoromethyl)pyrimidin-2- yl)amino)-1H-indazol-1- yl)acetamide | 461.2 |
| 101 | 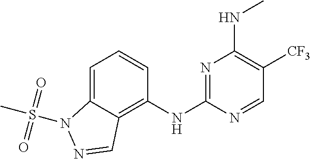 [SMILES: CNc1nc(Nc2cccc3c2cnn3S(C)(=O)=O)ncc1C(F)(F)F] | N4-cyclopropyl-N2-(1- (methylsulfonyl)-1H-indazol-4-yl)- 5-(trifluoromethyl)pyrimidine-2,4- diamine | 387.0 |
| 102 | 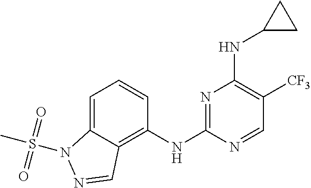 [SMILES: CS(=O)(=O)n1ncc2c(Nc3ncc(C(F)(F)F)c(NC4CC4)n3)cccc21] | N4-cyclopropyl-N2-(1- (methylsulfonyl)-1H-indazol-4-yl)- 5.(trifluoromethyl)pyrimidine-2,4- diamine | 413.2 |
| 103 | 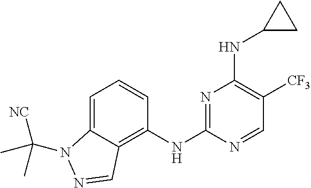 [SMILES: CC(C)(C#N)n1ncc2c(Nc3ncc(C(F)(F)F)c(NC4CC4)n3)cccc21] | (trifluoromethyl)pyrimidin-2- yl)amino)-1H-indazol-1-yl)-2- methylpropanenitrile | 402.2 |
| 104 | 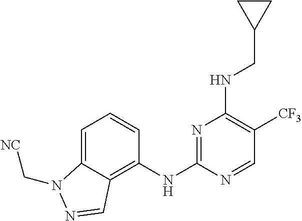 [SMILES: N#CCn1ncc2c(Nc3ncc(C(F)(F)F)c(NCC4CC4)n3)cccc21] | 2-(4-((4- ((cyclopropylmethyl)amino)-5- (trifluoromethyl)pyrimidin-2- yl)amino)-1H-indazol-1- yl)acetonitrile | 388.1 |
| 105 | 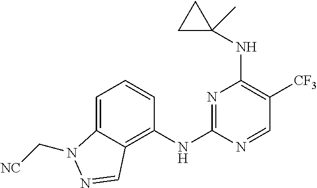 [SMILES: CC1(Nc2nc(Nc3cccc4c3cnn4CC#N)ncc2C(F)(F)F)CC1] | 2-(4-((4-((1- methylcyclopropyl)amino)-5- (trifluoromethyl)pyrimidin-2- yl)amino)-1H-indazol-1- yl)acetonitrile | 388.1 |
| 106 | 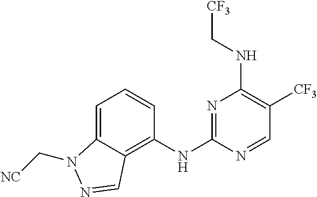 [SMILES: N#CCn1ncc2c(Nc3ncc(C(F)(F)F)c(NCC(F)(F)F)n3)cccc21] | 2-(4-((4-((2,2,2- trifluoroethyl)amino)-5- (trifluoromethyl)pyrimidin-2- yl)amino)-1H-indazol-1- yl)acetonitrile | 416.1 |
| 107 | 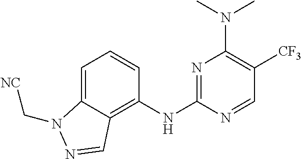 [SMILES: CN(C)c1nc(Nc2cccc3c2cnn3CC#N)ncc1C(F)(F)F] | 2-(4-((4-(dimethylamino)-5- (trifluoromethyl)pyrimidin-2- yl)amino)-1H-indazol-1- yl)acetonitrile | 362.1 |
| 108 | 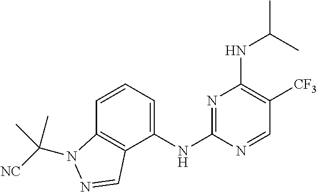 [SMILES: CC(C)Nc1nc(Nc2cccc3c2cnn3C(C)(C)C#N)ncc1C(F)(F)F] | 2-(4-((4-(isopropylamino)-5- (trifluoromethyl)pyrimidin-2- yl)amino)-1H-indazol-1-yl)-2- methylpropanenitrile | 404.3 |
| 109 | 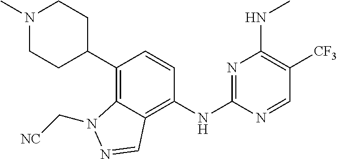 [SMILES: CNc1nc(Nc2ccc(C3CCN(C)CC3)c3c2cnn3CC#N)ncc1C(F)(F)F] | 2-(4-((4-(methylamino)-5- (trifluoromethyl)pyrimidin-2- yl)amino)-7-(1-methylpiperidin-4- yl)-1H-indazol-1-yl)acetonitrile | 445.2 |
| 110 | 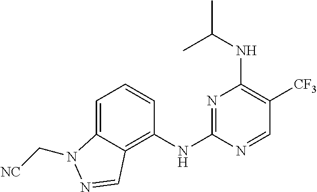 [SMILES: CC(C)Nc1nc(Nc2cccc3c2cnn3CC#N)ncc1C(F)(F)F] | 2-(4-((4-(isopropylamino)-5- (trifluoromethyl)pyrimidin-2- yl)amino)-1H-indazol-1- yl)acetonitrile | 376.1 |
| 111 | 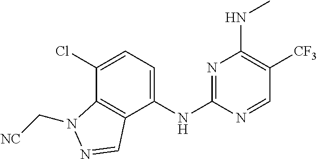 [SMILES: CNc1nc(Nc2ccc(Cl)c3c2cnn3CC#N)ncc1C(F)(F)F] | 2-(7-chloro-4-((4-(methylamino)-5- (trifluoromethyl)pyrimidin-2- yl)amino)-1H-indazol-1- yl)acetonitrile | 382.1 |
| 112 | 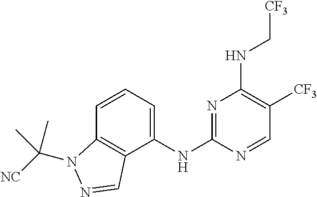 [SMILES: CC(C)(C#N)n1ncc2c(Nc3ncc(C(F)(F)F)c(NCC(F)(F)F)n3)cccc21] | 2-methyl-2-(4-((4-((2,2,2- trifluoroethyl)amino)-5- (trifluoromethyl)pyrimidin-2- yl)amino)-1H-indazol-1- yl)propanenitrile | 444.2 |
| 113 | 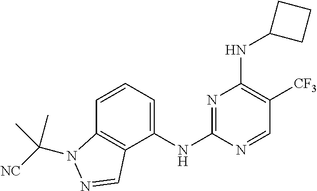 [SMILES: CC(C)(C#N)n1ncc2c(Nc3ncc(C(F)(F)F)c(NC4CCC4)n3)cccc21] | 2-(4-((4-(cyclobutylamino)-5- (trifluoromethyl)pyrimidin-2- yl)amino)-1H-indazol-1-yl)-2- methylpropanenitrile | 416.4 |
| 114 | 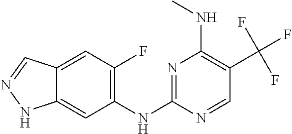 [SMILES: CNc1nc(Nc2cc3[nH]ncc3cc2F)ncc1C(F)(F)F] | N²-(5-fluoro-1H-indazol-6-yl)-N⁴- methyl-5- (trifluoromethyl)pyrimidine-2,4- diamine | 326.9 |
| 115 | 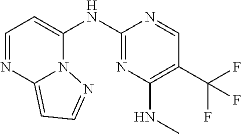 [SMILES: CNc1nc(Nc2ccnc3ccnn23)ncc1C(F)(F)F] | N⁴-methyl-N²-(pyrazolo[1,5- a]pyrimidin-7-yl)-5- (trifluoromethyl)pyrimidine-2,4- diamine | 309.9 |
| 116 | 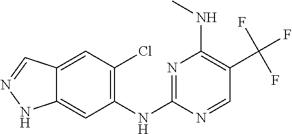 [SMILES: CNc1nc(Nc2cc3[nH]ncc3cc2Cl)ncc1C(F)(F)F] | N²-(5-chloro-1H-indazol-6-yl)-N⁴- methyl-5- (trifluoromethyl)pyrimidine-2,4- diamine | 343.0 |
| 117 | 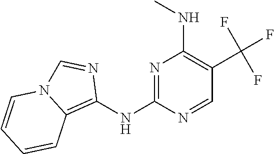 [SMILES: CNc1nc(Nc2ncn3ccccc23)ncc1C(F)(F)F] | N²-(imidazo[1,5-a]pyridin-1-yl)- N⁴-methyl-5- (trifluoromethyl)pyrimidine-2,4- diamine | 309.0 |
| 118 | 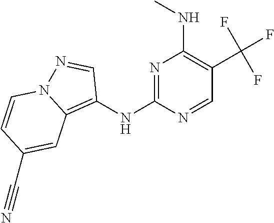 [SMILES: CNc1nc(Nc2cnn3ccc(C#N)cc23)ncc1C(F)(F)F] | 3-((4-(methylamino)-5- (trifluoromethyl)pyrimidin-2- yl)amino)pyrazolo[1,5-a]pyridine- 5-carbonitrile | 334.0 |
| 119 | 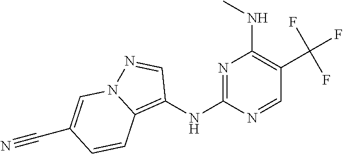 [SMILES: CNc1nc(Nc2cnn3cc(C#N)ccc23)ncc1C(F)(F)F] | 3-((4-(methylamino)-5- (trifluoromethyl)pyrimidin-2- yl)amino)pyrazolo[1,5-a]pyridine- 6-carbonitrile | 334.0 |
| 120 | 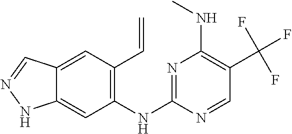 [SMILES: C=Cc1cc2cn[nH]c2cc1Nc1ncc(C(F)(F)F)c(NC)n1] | N⁴-methyl-5-(trifluoromethyl)-N²- (5-vinyl-1H-indazol-6- yl)pyrimidine-2,4-diamine | 335.1 |
| 121 | 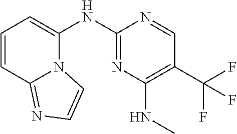 [SMILES: CNc1nc(Nc2cccc3nccn23)ncc1C(F)(F)F] | N²-(imidazo[1,2-a]pyridin-5-yl)- N⁴-methyl-5- (trifluoromethyl)pyrimidine-2,4- diamine | 309.4 |
| 122 | 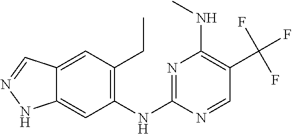 [SMILES: CCc1cc2cn[nH]c2cc1Nc1ncc(C(F)(F)F)c(NC)n1] | N²-(5-ethyl-1H-indazol-6-yl)-N⁴- methyl-5- (trifluoromethyl)pyrimidine-2,4- diamine | 337.1 |
| 123 | 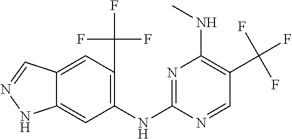 [SMILES: CNc1nc(Nc2cc3[nH]ncc3cc2C(F)(F)F)ncc1C(F)(F)F] | N⁴-methyl-5-(trifluoromethyl)-N²- (5-(trifluoromethyl)-1H-indazol-6- yl)pyrimidine-2,4-diamine | 377.1 |
| 124 | 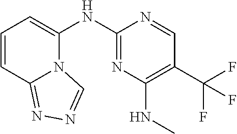 [SMILES: CNc1nc(Nc2cccc3nncn23)ncc1C(F)(F)F] | N²-([1,2,4]triazolo[4,3-a]pyridin-5- yl)-N⁴-methyl-5- (trifluoromethyl)pyrimidine-2,4- diamine | 309.9 |
| 125 | 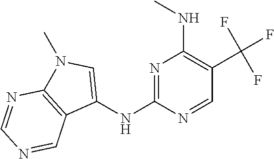 [SMILES: CNc1nc(Nc2cn(C)c3ncncc23)ncc1C(F)(F)F] | N⁴-methyl-N²-(7-methyl-7H- pyrrolo[2,3-d]pyrimidin-5-yl)-5- (trifluoromethyl)pyrimidine-2,4- diamine | 324.1 |
| 126 | 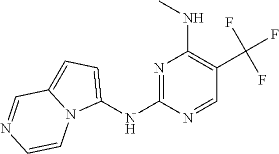 [SMILES: CNc1nc(Nc2ccc3cnccn23)ncc1C(F)(F)F] | N⁴-methyl-N²-(pyrrolo[1,2- a]pyrazin-6-yl)-5- (trifluoromethyl)pyrimidine-2,4- diamine | 309.3 |
| 127 | 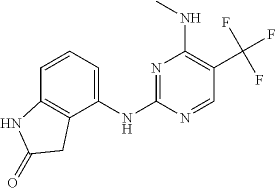 [SMILES: CNc1nc(Nc2cccc3c2CC(=O)N3)ncc1C(F)(F)F] | 4-((4-(methylamino)-5- (trifluoromethyl)pyrimidin-2- yl)amino)indolin-2-one | 324.3 |
| 128 | 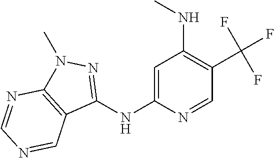 [SMILES: CNc1cc(Nc2nn(C)c3ncncc23)ncc1C(F)(F)F] | N⁴-methyl-N²-(1-methyl-1H- pyrazolo[3,4-d]pyrimidin-3-yl)-5- (trifluoromethyl)pyridine-2,4- diamine | 324.0 |
| 129 | 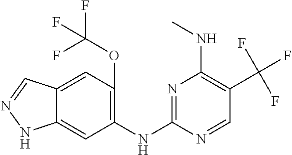 [SMILES: CNc1nc(Nc2cc3[nH]ncc3cc2OC(F)(F)F)ncc1C(F)(F)F] | N⁴-methyl-N²-(5- (trifluoromethoxy)-1H-indazol-6- yl)-5-(trifluoromethyl)pyrimidine- 2,4-diamine | 393.3 |
| 130 | 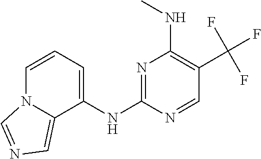 [SMILES: CNc1nc(Nc2cccn3cncc23)ncc1C(F)(F)F] | N²-(imidazo[1,5-a]pyridin-8-yl)- N⁴-methyl-5- (trifluoromethyl)pyrimidine-2,4- diamine | 309.3 |
| 131 | 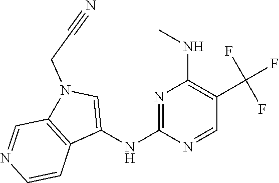 [SMILES: CNc1nc(Nc2cn(CC#N)c3cnccc23)ncc1C(F)(F)F] | 2-(3-((4-(methylamino)-5- (trifluoromethyl)pyrimidin-2- yl)amino)-1H-pyrrolo[2,3- c]pyridin-1-yl)acetonitrile | 348.0 |
| 132 | 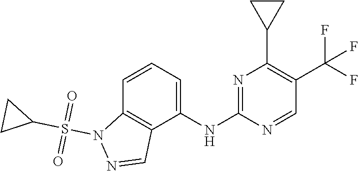 [SMILES: O=S(=O)(C1CC1)n1ncc2c(Nc3ncc(C(F)(F)F)c(C4CC4)n3)cccc21] | N-(4-cyclopropyl-5- (trifluoromethyl)pyrimidin-2-yl)-1- (cyclopropylsulfonyl)-1H-indazol- 4-amine | 424.1 |
| 133 | 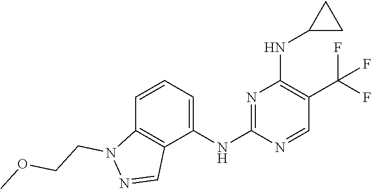 [SMILES: COCCn1ncc2c(Nc3ncc(C(F)(F)F)c(NC4CC4)n3)cccc21] | N⁴-cyclopropyl-N²-(1-(2- methoxyethyl)-1H-indazol-4-yl)-5- (trifluoromethyl)pyrimidine-2,4- diamine | 393.2 |
| 134 | 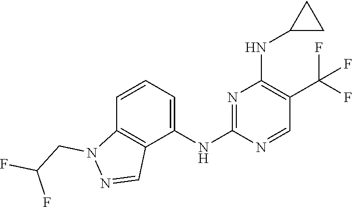 [SMILES: FC(F)Cn1ncc2c(Nc3ncc(C(F)(F)F)c(NC4CC4)n3)cccc21] | N⁴-cyclopropyl-N²-(1-(2,2- difluoroethyl)-1H-indazol-4-yl)-5- (trifluoromethyl)pyrimidine-2,4- diamine | 399.2 |
| 135 | 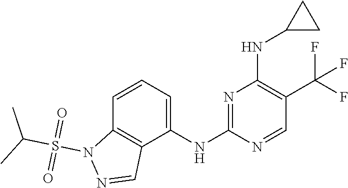 [SMILES: CC(C)S(=O)(=O)n1ncc2c(Nc3ncc(C(F)(F)F)c(NC4CC4)n3)cccc21] | N⁴-cyclopropyl-N²-(1- (isopropylsulfonyl)-1H-indazol-4- yl)-5-(trifluoromethyl)pyrimidine- 2,4-diamine | 441.2 |
| 136 | 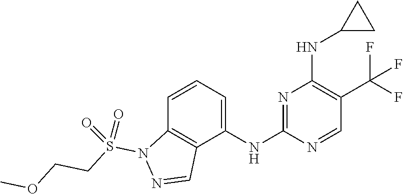 [SMILES: COCCS(=O)(=O)n1ncc2c(Nc3ncc(C(F)(F)F)c(NC4CC4)n3)cccc21] | N⁴-cyclopropyl-N²-(1-((2- methoxyethyl)sulfonyl)-1H- indazol-4-yl)-5- (trifluoromethyl)pyrimidine-2,4- diamine | 457.0 |
| 137 | 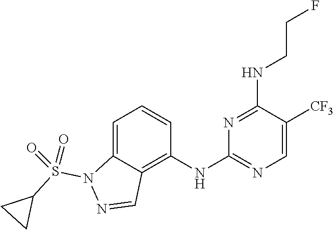 [SMILES: O=S(=O)(C1CC1)n1ncc2c(Nc3ncc(C(F)(F)F)c(NCCF)n3)cccc21] | N²-(1-(cyclopropylsulfonyl)-1H- indazol-4-yl)-N⁴-(2-fluoroethyl)-5- (trifluoromethyl) pyrimidine-2,4- diamine | 445.0 |
| 138 | 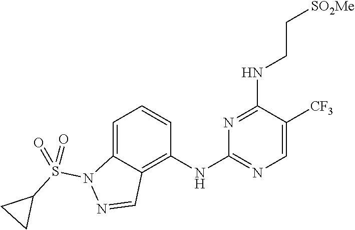 [SMILES: CS(=O)(=O)CCNc1nc(Nc2cccc3c2cnn3S(=O)(=O)C2CC2)ncc1C(F)(F)F] | N²-(1-(cyclopropylsulfonyl)-1H- indazol-4-yl)-N⁴-(2- (methylsulfonyl)ethyl)-5- (trifluoromethyl) pyrimidine-2,4- diamine | 505.0 |
| 139 | 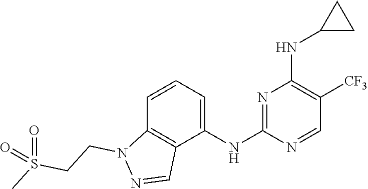 [SMILES: CS(=O)(=O)CCn1ncc2c(Nc3ncc(C(F)(F)F)c(NC4CC4)n3)cccc21] | N⁴-cyclopropyl-N²-(1-(2- (methylsulfonyl)ethyl)-1H-indazol- 4-yl)-5- (trifluoromethyl)pyrimidine-2,4- diamine | 441.0 |
| 140 | 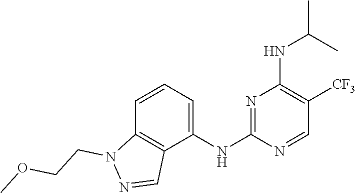 [SMILES: COCCn1ncc2c(Nc3ncc(C(F)(F)F)c(NC(C)C)n3)cccc21] | N⁴-isopropyl-N²-[1-(2- methoxyethyl)indazol-4-yl]-5- (trifluoromethyl)pyrimidine-2,4- diamine | 395.2 |
| 141 | 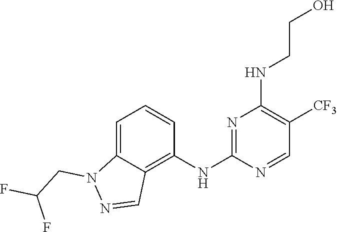 [SMILES: OCCNc1nc(Nc2cccc3c2cnn3CC(F)F)ncc1C(F)(F)F] | 2-[[2-[[1-(2,2- difluoroethyl)indazol-4-yl]amino]- 5-(trifluoromethyl)pyrimidin-4- yl]amino] ethanol | 403.2 |
| 142 | 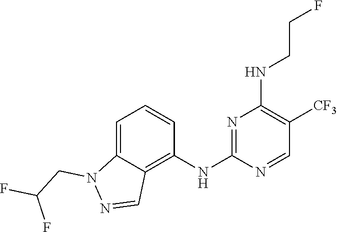 [SMILES: FCCNc1nc(Nc2cccc3c2cnn3CC(F)F)ncc1C(F)(F)F] | N²-[1-(2,2-difluoroethyl)indazol-4- yl]-N⁴-(2-fluoroethyl)-5- (trifluoromethyl) pyrimidine-2,4- diamine | 405.2 |
| 143 | 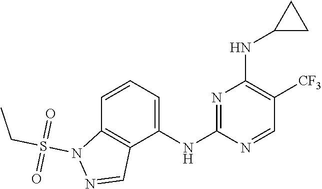 [SMILES: CCS(=O)(=O)n1ncc2c(Nc3ncc(C(F)(F)F)c(NC4CC4)n3)cccc21] | N⁴-cyclopropyl-N²-(1- ethylsulfonylindazol-4-yl)-5- (trifluoromethyl)pyrimidine-2,4- diamine | 427.0 |
| 144 | 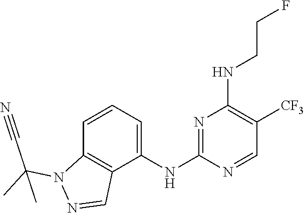 [SMILES: CC(C)(C#N)n1ncc2c(Nc3ncc(C(F)(F)F)c(NCCF)n3)cccc21] | 2-(4-((4-((2-fluoroethyl)amino)-5- (trifluoromethyl)pyrimidin-2- yl)amino)-1H-indazol-1-yl)-2- methylpropanenitrile | 408.0 |
| 145 | 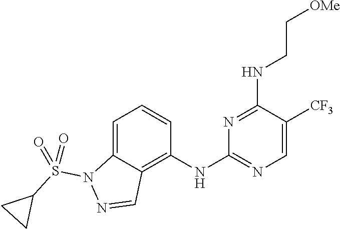 [SMILES: COCCNc1nc(Nc2cccc3c2cnn3S(=O)(=O)C2CC2)ncc1C(F)(F)F] | N²-(1-cyclopropylsulfonylindazol- 4-yl)-N⁴-(2-methoxyethyl)-5- (trifluoromethyl) pyrimidine-2,4- diamine | 457.1 |
| 146 | 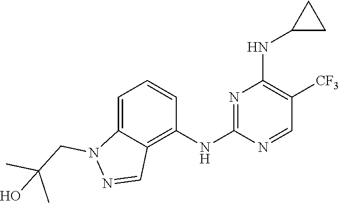 [SMILES: CC(C)(O)Cn1ncc2c(Nc3ncc(C(F)(F)F)c(NC4CC4)n3)cccc21] | 1-[4-[4-(cyclopropylamino)-5- (trifluoromethyl)pyrimidin-2- yl]amino]indazol-1-yl]-2-methyl- propan-2-ol | 407.1 |
| 147 | 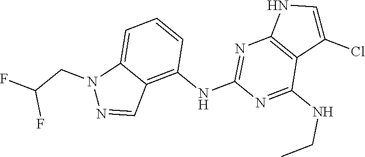 [SMILES: CCNc1nc(Nc2cccc3c2cnn3CC(F)F)nc2[nH]cc(Cl)c12] | 5-chloro-N²-(1-(2,2-difluoroethyl)- 1H-indazol-4-yl)-N⁴-ethyl-7H- pyrrolo[2,3-d]pyrimidine-2,4- diamine | 392.1 |
| 148 | 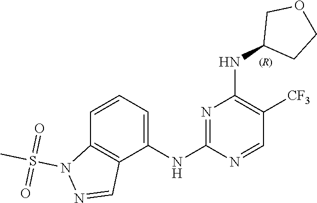 | (R)-N²-(1-(methylsulfonyl)-1H- indazol-4-yl)-N⁴-(tetrahydrofuran- 3-yl)-5- (trifluoromethyl)pyrimidine-2,4- diamine | 443.0 |
| 149 | 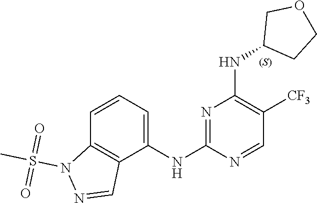 | (S)-N²-(1-(methylsulfonyl)-1H- indazol-4-yl)-N⁴-(tetrahydrofuran- 3-yl)-5- (trifluoromethyl)pyrimidine-2,4- diamine | 443.0 |
| 150 | 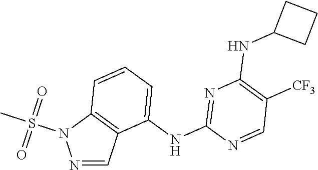 [SMILES: CS(=O)(=O)n1ncc2c(Nc3ncc(C(F)(F)F)c(NC4CCC4)n3)cccc21] | N⁴-cyclobutyl-N²-(1- methylsulfonylindazol-4-yl)-5- (trifluoromethyl)pyrimidine-2,4- diamine | 427.1 |
| 151 | 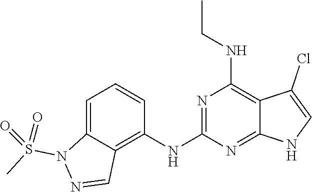 [SMILES: CCNc1nc(Nc2cccc3c2cnn3S(C)(=O)=O)nc2[nH]cc(Cl)c12] | 5-chloro-N⁴-ethyl-N²-(1- methylsulfonylindazol-4-yl)-7H- pyrrolo[2,3-d]pyrimidine-2,4- diamine | 406.0 |
| 152 | 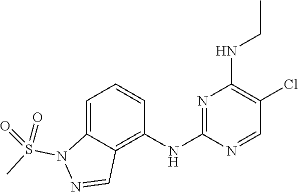 [SMILES: CCNc1nc(Nc2cccc3c2cnn3S(C)(=O)=O)ncc1Cl] | 5-chloro-N⁴-ethyl-N²-(1- (methylsulfonyl)-1H-indazol-4- yl)pyrimidine-2,4-diamine | 367.0 |
| 153 | 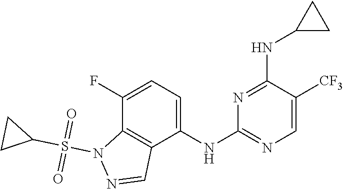 [SMILES: O=S(=O)(C1CC1)n1ncc2c(Nc3ncc(C(F)(F)F)c(NC4CC4)n3)ccc(F)c21] | N⁴-cyclopropyl-N²-(1- (cyclopropylsulfonyl)-7-fluoro-1H- indazol-4-yl)-5-(trifluoromethyl) pyrimidine-2,4-diamine | 457.0 |
| 154 | 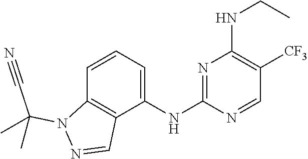 [SMILES: CCNc1nc(Nc2cccc3c2cnn3C(C)(C)C#N)ncc1C(F)(F)F] | 2-(4-((4-(ethylamino)-5- (trifluoromethyl)pyrimidin-2- yl)amino)-1H-indazol-1-yl)-2- methylpropanenitrile | 390.1 |
| 155 | 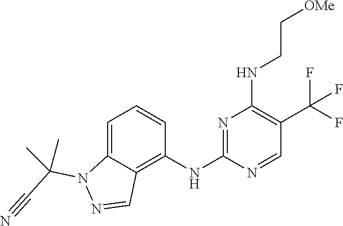 [SMILES: COCCNc1nc(Nc2cccc3c2cnn3C(C)(C)C#N)ncc1C(F)(F)F] | 2-[4-[[4-(2-methoxyethylamino)-5- (trifluoromethyl) pyrimidin-2- yl]amino]indazol-1-yl]-2-methyl- propanenitrile | 420.1 |
| 156 | 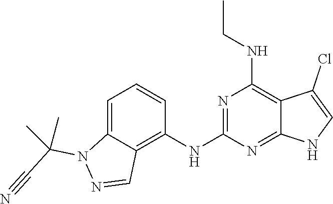 [SMILES: CCNc1nc(Nc2cccc3c2cnn3C(C)(C)C#N)nc2[nH]cc(Cl)c12] | 2-(4-((5-chloro-4-(ethylamino)-7H- pyrrolo[2,3-d]pyrimidin-2- yl)amino)-1H-indazol-1-yl)-2- methylpropanenitrile | 395.1 |
| 157 | 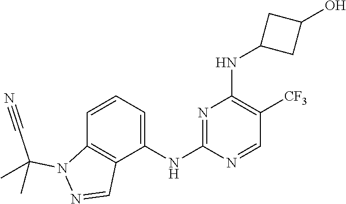 [SMILES: CC(C)(C#N)n1ncc2c(Nc3ncc(C(F)(F)F)c(NC4CC(O)C4)n3)cccc21] | 2-[4-[[4-[(3- hydroxycyclobutyl)amino]-5- (trifluoromethyl)pyrimidin-2- yl]amino]indazol-1-yl]-2-methyl- propanenitrile | 432.1 |
| 158 | 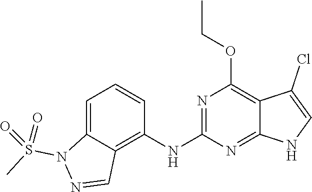 [SMILES: CCOc1nc(Nc2cccc3c2cnn3S(C)(=O)=O)nc2[nH]cc(Cl)c12] | 5-chloro-4-ethoxy-N-(1- methylsulfonylindazol-4-yl)-7H- pyrrolo [2,3-d]pyrimidin-2-amine | 407.1 |
| 159 | 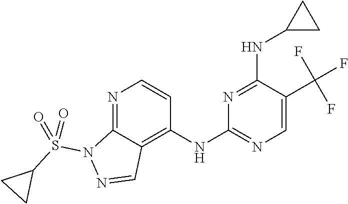 [SMILES: O=S(=O)(C1CC1)n1ncc2c(Nc3ncc(C(F)(F)F)c(NC4CC4)n3)ccnc21] | N⁴-cyclopropyl-N²-(1- (cyclopropylsulfonyl)-1H- pyrazolo[3,4-b]pyridin-4-yl)-5- (trifluoromethyl)pyrimidine-2,4- diamine | 440.0 |
| 160 | 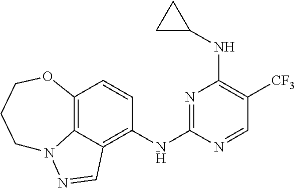 [SMILES: FC(F)(F)c1cnc(Nc2ccc3c4c2cnn4CCCO3)nc1NC1CC1] | N⁴-cyclopropyl-N²-(9-oxa-1,2- diazatricyclo[6.4.1.04,13]trideca- 2,4,6,8(13)-tetraen-5-yl)-5- (trifluoromethyl)pyrimidine-2,4- diamine | 391.1 |
| 161 | 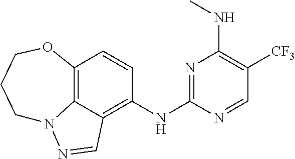 [SMILES: CNc1nc(Nc2ccc3c4c2cnn4CCCO3)ncc1C(F)(F)F] | N⁴-methyl-N²-(9-oxa-1,2- diazatricyclo[6.4.1.04,13]trideca- 2,4,6,8(13)-tetraen-5-yl)-5- (trifluoromethyl)pyrimidine-2,4- diamine | 365.1 |
| 162 | 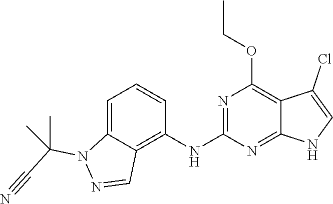 [SMILES: CCOc1nc(Nc2cccc3c2cnn3C(C)(C)C#N)nc2[nH]cc(Cl)c12] | 2-[4-[(S-chloro-4-ethoxy-7H- pyrrolo[2,3-d]pyrimidin-2- yl)amino]indazol-1-yl]-2-methyl- propanenitrile | 396.1 |
| 163 | 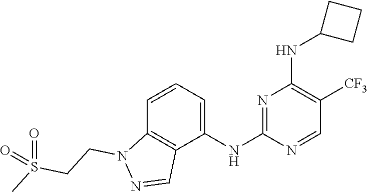 [SMILES: CS(=O)(=O)CCn1ncc2c(Nc3ncc(C(F)(F)F)c(NC4CCC4)n3)cccc21] | N⁴-cyclobutyl-N2-(1-(2- (methylsulfonyl)ethyl)-1H-indazol- 4-yl)-5- (trifluoromethyl)pyrimidine-2,4- diamine | 455.5 |
| 164 | 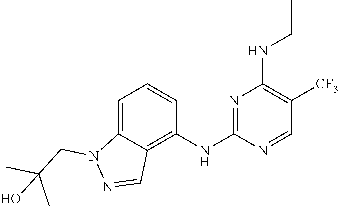 [SMILES: CCNc1nc(Nc2cccc3c2cnn3CC(C)(C)O)ncc1C(F)(F)F] | 1-[4-[[4-(ethylamino)-5- (trifluoromethyl) pyrimidin-2- yl]amino]indazol-1-yl]-2-methyl- propan-2-ol | 395.1 |
| 165 | 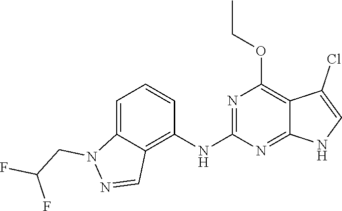 [SMILES: CCOc1nc(Nc2cccc3c2cnn3CC(F)F)nc2[nH]cc(Cl)c12] | 5-chloro-N-[1-(2,2- difluoroethyl)indazol-4-yl]-4- ethoxy-7H-pyrrolo[2,3- d]pyrimidin-2-amine | 393.0 |
| 166 | 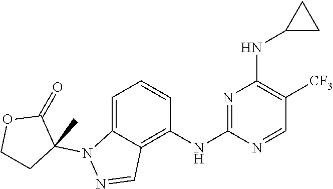 [SMILES: C[C@@]1(n2ncc3c(Nc4ncc(C(F)(F)F)c(NC5CC5)n4)cccc32)CCOC1=O] | (R)-3-(4-((4-(cyclopropylamino)-5- (trifluoromethyl)pyrimidin-2- yl)amino)-1H-indazol-1-yl)-3- methyldihydrofuran-2(3H)-one | 433.3 |
| 167 | 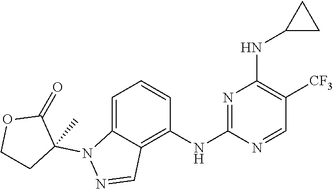 [SMILES: C[C@]1(n2ncc3c(Nc4ncc(C(F)(F)F)c(NC5CC5)n4)cccc32)CCOC1=O] | (S)-3-(4-((4-(cyclopropylamino)-5- (trifluoromethyl)pyrimidin-2- yl)amino)-1H-indazol-1-yl)-3- methyldihydrofuran-2(3H)-one | 433.3 |
| 168 | 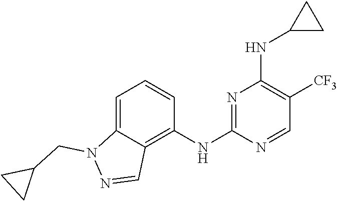 [SMILES: FC(F)(F)c1cnc(Nc2cccc3c2cnn3CC2CC2)nc1NC1CC1] | N⁴-cyclopropyl-N²-(1- (cyclopropylmethyl)-1H-indazol-4- yl)-5-(trifluoromethyl)pyrimidine- 2,4-diamine | 389.1 |
| 169 | 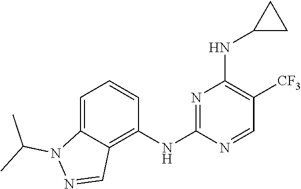 [SMILES: CC(C)n1ncc2c(Nc3ncc(C(F)(F)F)c(NC4CC4)n3)cccc21] | N⁴-cyclopropyl-N²-(1-isopropyl- 1H-indazol-4-yl)-5- (trifluoromethyl) pyrimidine-2,4- diamine | 377.2 |
| 170 | 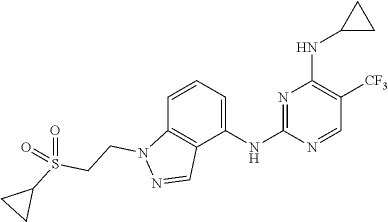 [SMILES: O=S(=O)(CCn1ncc2c(Nc3ncc(C(F)(F)F)c(NC4CC4)n3)cccc21)C1CC1] | N⁴-cyclopropyl-N²-(1-(2- (cyclopropylsulfonyl)ethyl)-1H- indazol-4-yl)-5- (trifluoromethyl)pyrimidine-2,4- diamine | 489.1 |
| 171 | 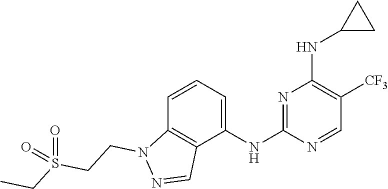 [SMILES: CCS(=O)(=O)CCn1ncc2c(Nc3ncc(C(F)(F)F)c(NC4CC4)n3)cccc21] | N⁴-cyclopropyl-N²-(1-(2- (ethylsulfonyl)ethyl)-1H-indazol-4- yl)-5-(trifluoromethyl)pyrimidine- 2,4-diamine | 455.1 |
| 172 | 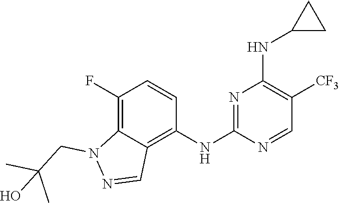 [SMILES: CC(C)(O)Cn1ncc2c(Nc3ncc(C(F)(F)F)c(NC4CC4)n3)ccc(F)c21] | 1-(4-((4-(cyclopropylamino)-5- (trifluoromethyl)pyrimidin-2- yl)amino)-7-fluoro-1H-indazol-1- yl)-2-methylpropan-2-ol | 425.1 |
| 173 | 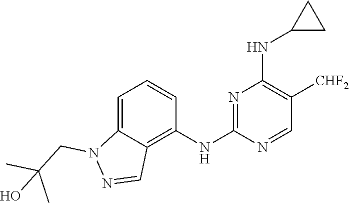 [SMILES: CC(C)(O)Cn1ncc2c(Nc3ncc(C(F)F)c(NC4CC4)n3)cccc21] | 1-[4-[[4-(cyclopropylamino)-5- (difluoromethyl)pyrimidin-2- yllamino]indazol-1-yl]-2-methyl- propan-2-ol | 389.3 |
| 174 | 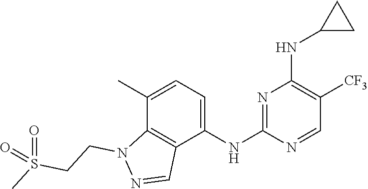 [SMILES: Cc1ccc(Nc2ncc(C(F)(F)F)c(NC3CC3)n2)c2cnn(CCS(C)(=O)=O)c12] | N⁴-cyclopropyl-N²-(7-methyl-1-(2- (methylsulfonyl)ethyl)-1H-indazol- 4-yl)-5- (trifluoromethyl)pyrimidine-2,4- diamine | 455.1 |
| 175 | 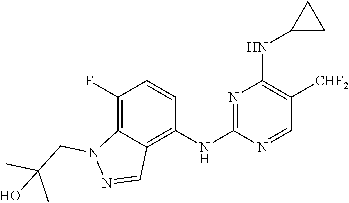 [SMILES: CC(C)(O)Cn1ncc2c(Nc3ncc(C(F)F)c(NC4CC4)n3)ccc(F)c21] | 1-[4-[[4-(cyclopropylamino)-5- (difluoromethyl)pyrimidin-2- yl]amino]-7-fluoro-indazol-1-yl]-2- methyl-propan-2-ol | 407.0 |
| 176 | 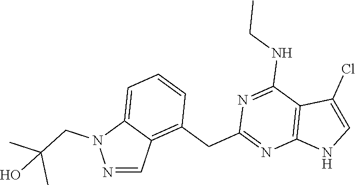 [SMILES: CCNc1nc(Cc2cccc3c2cnn3CC(C)(C)O)nc2[nH]cc(Cl)c12] | 1-[4-[[5-chloro-4-(ethylamino)-7H- pyrrolo[2,3-d]pyrimidin-2- yl]aminolindazol-1-yl]-2-methyl- propan-2-ol | 400.1 |
| 177 | 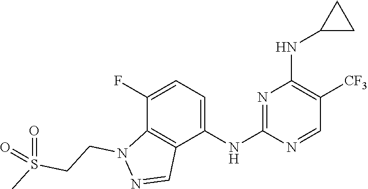 [SMILES: CS(=O)(=O)CCn1ncc2c(Nc3ncc(C(F)(F)F)c(NC4CC4)n3)ccc(F)c21] | N⁴-cyclopropyl-N²-[7-fluoro-1-(2- methylsulfonylethyl)indazol-4-yl]- 5-(trifluoromethyl)pyrimidine-2,4- diamine | 459.1 |
| 178 | 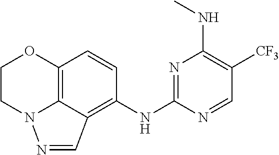 [SMILES: CNc1nc(Nc2ccc3c4c2cnn4CCO3)ncc1C(F)(F)F] | N⁴-methyl-N²-(9-oxa-1,2- diazatricyclo[6.3.1.04,12]dodeca- 2,4,6,8(12)-tetraen-5-yl)-5- (trifluoromethyl)pyrimidine-2,4- diamine | 351.3 |
| 179 | 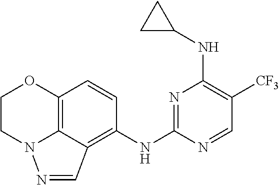 [SMILES: FC(F)(F)c1cnc(Nc2ccc3c4c2cnn4CCO3)nc1NC1CC1] | N⁴-cyclopropyl-N²-(2,3-dihydro- [1,4]oxazino[2,3,4-hi]indazol-7-yl)- 5-(trifluoromethyl)pyrimidine-2,4- diamine | 377.1 |
| 180 | 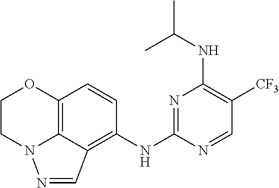 [SMILES: CC(C)Nc1nc(Nc2ccc3c4c2cnn4CCO3)ncc1C(F)(F)F] | N⁴-isopropyl-N²-(9-oxa-1,2- diazatricyclo[6.3.1.04, 12]dodeca- 2,4,6,8(12)-tetraen-5-yl)-5- (trifluoromethyl)pyrimidine-2,4- diamine | 379.0 |
| 181 | 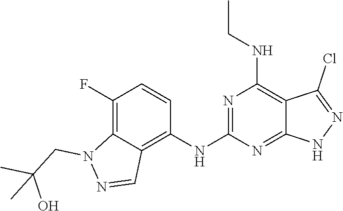 [SMILES: CCNc1nc(Nc2ccc(F)c3c2cnn3CC(C)(C)O)nc2[nH]nc(Cl)c12] | 1-(4-((3-chloro-4-(ethylamino)-1H- pyrazolo[3,4-d]pyrimidin-6- yl)amino)-7-fluoro-1H-indazol-1- yl)-2-methylpropan-2-ol | 419.1 |
| 182 | 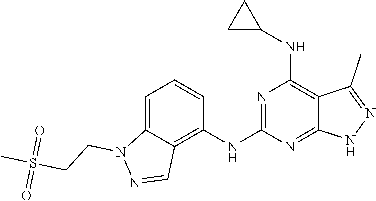 [SMILES: Cc1n[nH]c2nc(Nc3cccc4c3cnn4CCS(C)(=O)=O)nc(NC3CC3)c12] | N⁴-cyclopropyl-3-methyl-N⁶-[1-(2- methylsulfonylethyl)indazol-4-yl]- 1H-pyrazolo[3,4-d]pyrimidine-4,6- diamine | 427.1 |
| 183 | 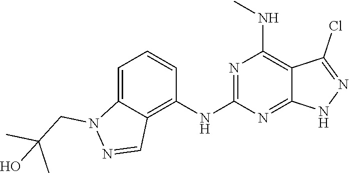 [SMILES: CNc1nc(Nc2cccc3c2cnn3CC(C)(C)O)nc2[nH]nc(Cl)c12] | 1-[4-[[3-chloro-4-(methylamino)- 1H-pyrazolo[3,4-d]pyrimidin-6- ylamino]indazol-1-yl]-2-methyl- propan-2-ol | 387.1 |
| 184 | 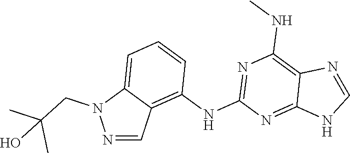 [SMILES: CNc1nc(Nc2cccc3c2cnn3CC(C)(C)O)nc2[nH]cnc12] | 2-methyl-1-[4-[6-(methylamino)- 9H-purin-2-yl]aminoJindazol-1- yl]propan-2-ol | 353.4 |
| 185 | 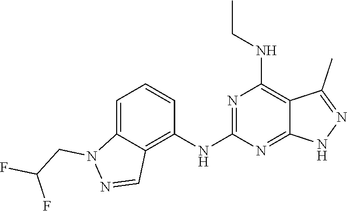 [SMILES: CCNc1nc(Nc2cccc3c2cnn3CC(F)F)nc2[nH]nc(C)c12] | N⁴-(1-(2,2-difluoroethyl)-1H- indazol-4-yl)-N⁴-ethyl-3-methyl- 1H-pyrazolo[3,4-d]pyrimidine-4,6- diamine | 373.2 |
| 186 | 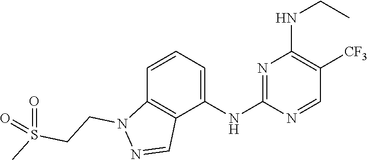 [SMILES: CCNc1nc(Nc2cccc3c2cnn3CCS(C)(=O)=O)ncc1C(F)(F)F] | N⁴-ethyl-N²-[1-(2- methylsulfonylethyl) indazol-4-yl]- 5-(trifluoromethyl) pyrimidine-2,4- diamine | 429.0 |
| 187 | 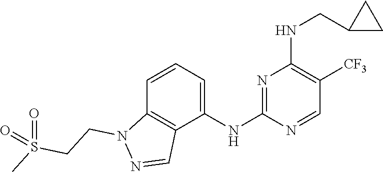 [SMILES: CS(=O)(=O)CCn1ncc2c(Nc3ncc(C(F)(F)F)c(NCC4CC4)n3)cccc21] | N⁴-(cyclopropylmethyl)-N²-(1-(2- (methylsulfonyl)ethyl)-1H-indazol- 4-yl)-5- (trifluoromethyl)pyrimidine-2,4- diamine | 455.2 |
| 188 | 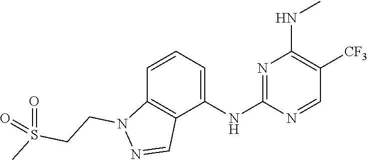 [SMILES: CNc1nc(Nc2cccc3c2cnn3CCS(C)(=O)=O)ncc1C(F)(F)F] | N⁴-methyl-N²-[1-(2- methylsulfonylethyl) indazol-4-yl]- 5-(trifluoromethyl) pyrimidine-2,4- diamine | 415.0 |
| 189 | 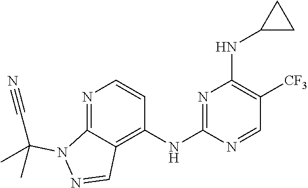 [SMILES: CC(C)(C#N)n1ncc2c(Nc3ncc(C(F)(F)F)c(NC4CC4)n3)ccnc21] | 2-(4-((4-(cyclopropylamino)-5- (trifluoromethyl)pyrimidin-2- yl)amino)-1H-pyrazolo[3,4- b]pyridin-1-yl)-2- methylpropanenitrile | 403.2 |
| 190 | 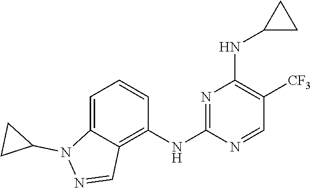 [SMILES: FC(F)(F)c1cnc(Nc2cccc3c2cnn3C2CC2)nc1NC1CC1] | N⁴-cyclopropyl-N²-(1-cyclopropyl- 1H-indazol-4-yl)-5- (trifluoromethyl) pyrimidine-2,4- diamine | 375.0 |
| 191 | 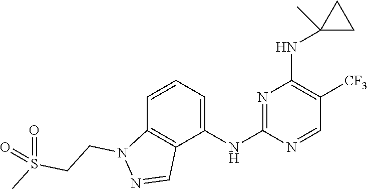 [SMILES: CC1(Nc2nc(Nc3cccc4c3cnn4CCS(C)(=O)=O)ncc2C(F)(F)F)CC1] | N⁴-(1-methylcyclopropyl)-N²-(1- (2-(methylsulfonyl)ethyl)-1H- indazol-4-yl)-5- (trifluoromethyl)pyrimidine-2,4- diamine | 477.1 |
| 192 | 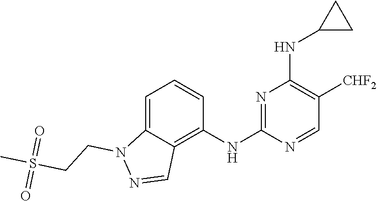 [SMILES: CS(=O)(=O)CCn1ncc2c(Nc3ncc(C(F)F)c(NC4CC4)n3)cccc21] | N⁴-cyclopropyl-5-(difluoromethyl)- N2-[1-(2- methylsulfonylethyl)indazol-4- yl]pyrimidine-2,4-diamine | 423.2 |
| 193 | 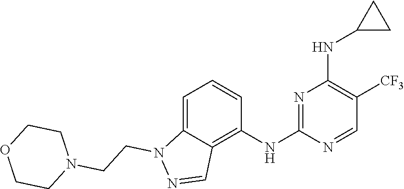 [SMILES: FC(F)(F)c1cnc(Nc2cccc3c2cnn3CCN2CCOCC2)nc1NC1CC1] | N⁴-cyclopropyl-N²-(1-(2- morpholinoethyl)-1H-indazol-4-yl)- 5-(trifluoromethyl)pyrimidine-2,4- diamine | 448.1 |
| 194 | 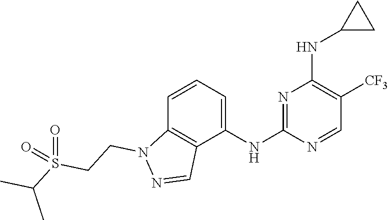 [SMILES: CC(C)S(=O)(=O)CCn1ncc2c(Nc3ncc(C(F)(F)F)c(NC4CC4)n3)cccc21] | N⁴-cyclopropyl-N²-(1-(2- (isopropylsulfonyl) ethyl)-1H- indazol-4-yl)-5- (trifluoromethyl)pyrimidine-2,4- diamine | 469.1 |
| 195 | 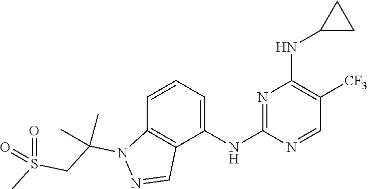 [SMILES: CC(C)(CS(C)(=O)=O)n1ncc2c(Nc3ncc(C(F)(F)F)c(NC4CC4)n3)cccc21] | N⁴-cyclopropyl-N²-(1-(2-methyl-1- (methylsulfonyl)propan-2-yl)-1H- indazol-4-yl)-5- (trifluoromethyl)pyrimidine-2,4- diamine | 469.3 |
| 196 | 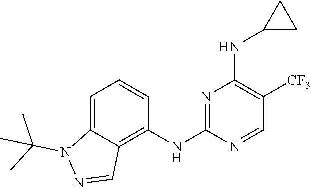 [SMILES: CC(C)(C)n1ncc2c(Nc3ncc(C(F)(F)F)c(NC4CC4)n3)cccc21] | N²-(1-tert-butylindazol-4-yl)-N⁴- cyclopropyl-5- (trifluoromethyl)pyrimidine-2,4- diamine | 391.1 |
| 197 | 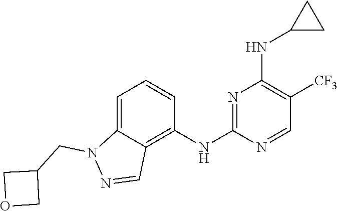 [SMILES: FC(F)(F)c1cnc(Nc2cccc3c2cnn3CC2COC2)nc1NC1CC1] | N⁴-cyclopropyl-N²-(1-(oxetan-3- ylmethyl)-1H-indazol-4-yl)-5- (trifluoromethyl)pyrimidine-2,4- diamine | 405.1 |
| 198 | 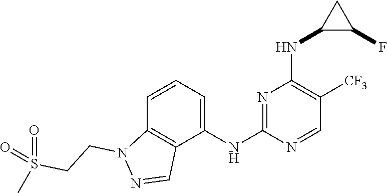 | N⁴-((1S,2R)-2-fluorocyclopropyl)- N²-(1-(2-(methylsulfonyl)ethyl)- 1H-indazol-4-yl)-5- (trifluoromethyl)pyrimidine-2,4- diamine (cis isomer) | 459.1 |
| 199 | 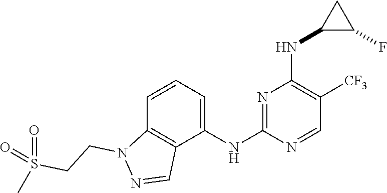 | N⁴-((1S,2S)-2-fluorocyclopropyl)- N² (1-(2-(methylsulfonyl)ethyl)- 1H-indazol-4-yl)-5- (trifluoromethyl)pyrimidine-2,4- diamine (trans isomer) | 459.1 |
| 200 | 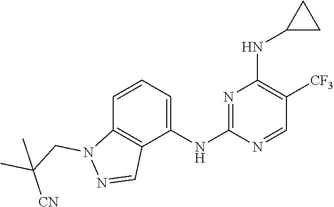 [SMILES: CC(C)(C#N)Cn1ncc2c(Nc3ncc(C(F)(F)F)c(NC4CC4)n3)cccc21] | 3-[4-[4-(cyclopropylamino)-5- (trifluoromethyl)pyrimidin-2- yl]amino]indazol-1-yl]-2,2- dimethyl-propanenitrile | 416.2 |
| 201 | 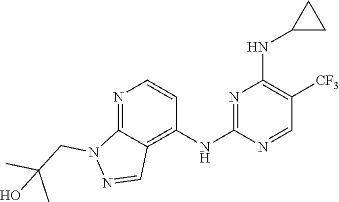 [SMILES: CC(C)(O)Cn1ncc2c(Nc3ncc(C(F)(F)F)c(NC4CC4)n3)ccnc21] | 1-(4-((4-(cyclopropylamino)-5- (trifluoromethyl)pyrimidin-2- yl)amino)-1H-pyrazolo[3,4- b]pyridin-1-yl)-2-methylpropan-2- ol | 408.2 |
| 202 | 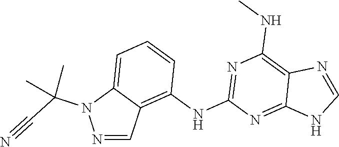 [SMILES: CNc1nc(Nc2cccc3c2cnn3C(C)(C)C#N)nc2[nH]cnc12] | 2-methyl-2-[4-[[6-(methylamino)- 9H-purin-2-yl]amino]indazol-1- yl]propanenitrile | 348.2 |
| 203 | 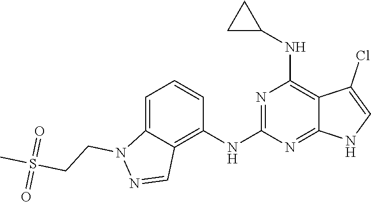 [SMILES: CS(=O)(=O)CCn1ncc2c(Nc3nc(NC4CC4)c4c(Cl)c[nH]c4n3)cccc21] | 5-chloro-N⁴-cyclopropyl-N²-(1-(2- (methylsulfonyl)ethyl)-1H-indazol- 4-yl)-7H-pyrrolo[2,3-d]pyrimidine- 2,4-diamine | 446.2 |
| 204 | 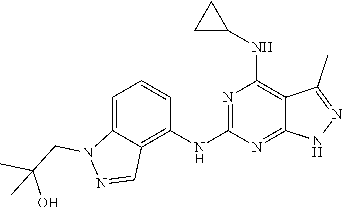 [SMILES: Cc1n[nH]c2nc(Nc3cccc4c3cnn4CC(C)(C)O)nc(NC3CC3)c12] | 1-[4-[[4-(cyclopropylamino)-3- methyl-1H-pyrazolo[3,4- d]pyrimidin-6-yl]amino]indazol-1- yl]-2-methyl-propan-2-ol | 393.3 |
| 205 | 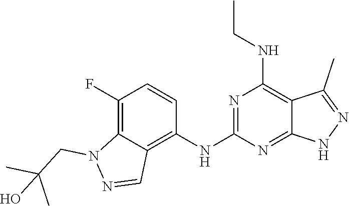 [SMILES: CCNc1nc(Nc2ccc(F)c3c2cnn3CC(C)(C)O)nc2[nH]nc(C)c12] | 1-[4-[[4-(ethylamino)-3-methyl- 1H-pyrazolo[3,4-d]pyrimidin-6- yl]amino]-7-fluoro-indazol-1-yl]-2- methyl-propan-2-ol | 399.3 |

### Example 1

### Synthesis of N2-(1H-indazol-6-yl)-N4-methyl-5-(trifluoromethyl)pyrimidine-2,4-diamine

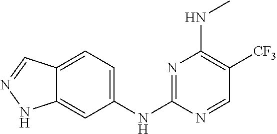 [SMILES: CNc1nc(Nc2ccc3cn[nH]c3c2)ncc1C(F)(F)F]

### Step 1

To a 0° C. solution of 2,4-dichloro-5-trifluoromethylpyrimidine (6.00 g, 27.1 mmol, 1.00 eq) in methanol (30.0 mL) were added triethylamine (4.24 mL, 30.40 mmol, 1.10 eq) and a solution of methylamine in absolute ethanol (33%, 4.18 mL, 29.80 mmol, 1.10 eq). The reaction was allowed to warm to room temperature and stirred for 3 h under argon atmosphere. The reaction mixture was concentrated and then partitioned between EtOAc (50 mL) and a saturated solution of NaHCO₃ (30 mL). The aqueous layer was extracted with EtOAc (50 mL×2). The combined organic layers were washed with brine (100 mL), dried over anhydrous MgSO₄, filtered, and concentrated under reduced pressure. The crude product was purified by flash chromatography on silica gel column using 0-10% EtOAc/Hexane as eluent, to provide 2-chloro-N-methyl-5-(trifluoromethyl)pyrimidin-4-amine (Intermediate 1-a) as a white solid (3.2 g, 56%). LCMS (ESI) m/z 212.1 [M+H]⁺.

### Step 2

A mixture of 2-chloro-N-methyl-5-(trifluoromethyl)pyrimidin-4-amine (Intermediate 1-a) (0.10 g, 0.27 mmol, 1.00 eq), 1H-indazol-6-amine (0.63 g, 0.47 mmol, 1.00 eq), and trifluoroacetic acid (0.11 mL, 1.42 mmol, 3.00 eq) in 2,2,2-trifluorethanol (3.0 mL) was stirred at 80° C. for 3 h. The reaction was cooled to room temperature and the mixture was diluted with toluene (2 ml) and then concentrated under reduced pressure. To the resulting residue in CH₂Cl₂ (5.0 mL) was added triethylamine (0.38 mL, 2.74 mml, 10.00 eq) and the mixture was stirred at 20° C. for 30 min. The resulting mixture was concentrated under reduced pressure, the remaining residue was purified by flash chromatography on silica gel column using 0-90% EtOAc/Hexane as eluent, to provide the title product as a white solid (36.0 mg, 25%). ¹H NMR (400 MHz, DMSO-d₆): δ ppm 3.00 (d, J=4.2 Hz, 3H), 7.20 (s, 1H), 7.31 (d, J=8.8 Hz, 1H), 7.60 (d, J=8.7 Hz, 1H), 7.92 (s, 1H), 8.21 (s, 1H), 8.33 (s, 1H), 9.80 (s, 1H), 12.86 (s, 1H); ¹H NMR (376 MHz, DMSO-d₆) δ ppm −60.34 (s, 3F); LCMS (ESI) m/z 309.1 [M+H]+.

### Example 2

### Synthesis of N2-(1H-indazol-6-yl)-N4-methyl-5-(trifluoromethyl)pyrimidine-2,4-diamine

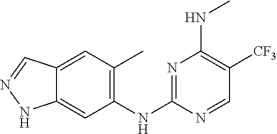 [SMILES: CNc1nc(Nc2cc3[nH]ncc3cc2C)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 5-methyl-1H-indazol-6-amine was used in place of 1H-indazol-6-amine. The crude product was purified by flash chromatography on silica gel column using 0-90% EtOAc/Hexane as eluent, to provide the title compound as a white powder (25.0 mg, 16%). ¹H NMR (400 MHz, DMSO-d₆) δ ppm 2.31 (s, 3H), 283 (d, J=4.4 Hz, 3H), 7.03 (d, J=5.0 Hz, 1H), 7.54 (s, 1H), 7.79 (s, 1H), 7.91 (s, 1H), 8.11 (s, 1H), 8.75 (s, 1H), 12.82 (s, 1H); ¹⁹F NMR (376 MHz, DMSO-d₆) δ ppm −60.16 (s, 3F); LCMS (ESI) m/z 323.1 [M+H]⁺.

### Example 3

### Synthesis of N2-(5-methoxy-1H-indazol-6-yl)-N4-methyl-5-(trifluoromethyl)pyrimidine-2,4-diamine

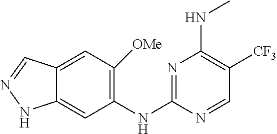 [SMILES: CNc1nc(Nc2cc3[nH]ncc3cc2OC)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 5-methoxy-1H-indazol-6-amine was used in place of 1H-indazol-6-amine. The crude product was purified by flash chromatography on silica gel column using 0-90% EtOAc/Hexane as eluent, to provide the title compound as a white powder (20.0 mg, 13%). H NMR (400 MHz, DMSO-d₆) δ ppm 2.99 (d, J=4.4 Hz, 3H), 3.92 (s, 3H), 7.25 (s, 1H), 7.30 (m, 1H), 7.88 (t, J=1.3 Hz, 1H), 8.06 (s, 1H), 8.23 (s, 1H), 8.60 (s, 1H), 12.84 (s, 1H); ¹⁹F NMR (376 MHz, DMSO-d₆) δ ppm −60.60 (s, 3F); LCMS (ESI) m/z 339.1 [M+H]⁺.

### Example 4

### Synthesis of N2-(7-methoxy-1H-indazol-6-yl)-N4-methyl-5-(trifluoromethyl)pyrimidine-2,4-diamine

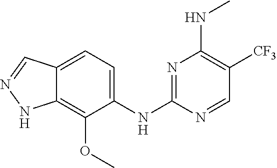 [SMILES: CNc1nc(Nc2ccc3cn[nH]c3c2OC)ncc1C(F)(F)F]

### Step 1

To a stirring solution of 4-bromo-2-fluoro-3-methoxybenzaldehyde (300 mg, 1.26 mmol, 1.00 eq) in DME (6.30 mL), was added hydrazine monohydrate (184 μL, 3.78 mmol, 3.00 eq). The resulting suspension was warmed to 90° C. and stirred for 1 h under argon atmosphere. After hydrazone formation was confirmed (LCMS (ESI) m/z 249.0 [M+H]⁺), the reaction mixture was warmed to 150° C. and stirred for 48 h. The reaction mixture was cooled to room temperature, poured into water (10 mL), and extracted with EtOAc (10 mL×3). The combined organic layers were washed with brine (20 mL), dried over anhydrous MgSO₄, filtered, and the volatiles removed under reduced pressure. The remaining residue nvc was purified by flash chromatography on silica gel column using 0-25% EtOAc/Hexanes as eluent, to provide 6-bromo-7-methoxy-1H-indazole (Intermediate 4-a) as a pale-beige solid (0.15 g, 53%). LCMS (ESI) m/z 228.9 [M+H]⁺.

### Step 2

To a stirring solution of 6-bromo-7-methoxy-1H-indazole (Intermediate 4-a) (152 mg, 669 μmol, 1.00 eq) in CH₂Cl₂ (2.70 mL), was added 3,4-dihydro-2H-pyran (183 μL, 2.01 mmol, 3.00 eq) and PTSA (12.7 mg, 66.9 μmol, 0.10 eq). The reaction mixture was stirred for 3 h at room temperature under argon atmosphere. The reaction mixture was partitioned between CH₂C₂ (10 mL) and a saturated solution of NaHCO₃ (10 mL). The layers were separated, and the aqueous layer was washed with CH₂Cl₂ (10 mL). The combined organic layers were dried over anhydrous MgSO₄, filtered, and the volatiles removed under reduced pressure. The crude product was purified by flash chromatography on silica gel column using 0-10% EtOAc/Hexanes as eluent, to provide 6-bromo-7-methoxy-1-(tetrahydro-2H-pyran-2-yl)-1H-indazole (Intermediate 4-b) as a clear oil (0.15 g, 74%). LCMS (ESI) m/z 312.9 [M+H]⁺.

### Step 3

To a stirring solution of 6-bromo-7-methoxy-1-(tetrahydro-2H-pyran-2-yl)-1H-indazole (Intermediate 4-b) (120 mg, 386 μmol, 1.00 eq) in anhydrous dioxane (3.90 mL) sparged with argon, was added tert-butyl carbamate (69.1 mg, 578 μmol, 1.50 eq), Cs₂CO₃ (251 mg, 771 μmol, 2.00 eq), Pd₂(dba)₃ (36.4 mg, 38.6 μmol, 0.10 eq), and Xantphos (45.5 mg, 77.1 μmol, 0.20 eq). The reaction mixture was degassed under reduced pressure with backflow of argon (3×), warmed to 100° C., and stirred for 2 h. The reaction mixture was cooled to room temperature, filtered over Celite, and concentrated under reduced pressure. The crude product was purified by flash chromatography on silica gel column using 0-20% EtOAc/Hexanes as eluent, to provide tert-butyl (7-methoxy-1-(tetrahydro-2H-pyran-2-yl)-1H-indazol-6-yl)carbamate (Intermediate 4-c) as a yellow solid (0.11 g, 84%). LCMS (ESI) m/z 348.1 [M+H]⁺.

### Step 4

To a stirring solution of tert-butyl (7-methoxy-1-(tetrahydro-2H-pyran-2-yl)-1H-indazol-6-yl)carbamate (Intermediate 4-c) (140 mg, 403 μmol, 1.00 eq) in CH₂Cl₂ (4.00 mL), was added TFA (617 μL, 8.06 mmol, 20.0 eq). The reaction mixture was stirred at room temperature for 18 h. The volatiles were removed under reduced pressure to provide 7-methoxy-1H-indazol-6-amine (Intermediate 4-d) as a brown oil (0.16 g, crude). LCMS (ESI) m/z 164.0 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 7-methoxy-1H-indazol-6-amine (Intermediate 4-d) was used in place of 1H-indazol-6-amine. The crude product was purified by reverse phase chromatography on a C18 column using 0-30% MeCN/H₂O (0.1% FA) as eluent, to provide the title compound as an off-white solid (38.2 mg, 28%). ¹H NMR (400 MHz, DMSO-d₆) δ ppm 2.84 (d, J=4.4 Hz, 3H), 3.92 (s, 3H), 7.08-7.05 (m, 1H), 7.41 (d, J=8.6 Hz, 1H), 7.61 (d, J=8.6 Hz, 1H), 8.03 (d, J=1.4 Hz, 1H), 8.11 (s, 1H), 8.60 (s, 1H), 13.19 (s, 1H); ¹⁹F NMR (376 MHz, DMSO-d₆) δ ppm −60.2 (s, 3F), LCMS (ESI) m/z 339.0 [M+H]⁺.

### Example 5

### Synthesis of N4-methyl-N2-(7-methyl-1H-indazol-6-yl)-5-(trifluoromethyl)pyrimidine-2,4-diamine

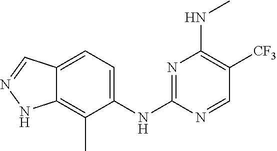 [SMILES: CNc1nc(Nc2ccc3cn[nH]c3c2C)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 7-methyl-1H-indazol-6-amine was used in place of 1H-indazol-6-amine. The crude product was purified by flash chromatography on silica gel column using 0-90% EtOAc/Hexane as eluent, to provide title compound as a white powder (30.0 mg, 20%). ¹H NMR (400 MHz, DMSO-d₆) δ ppm 2.36 (s, 3H), 2.76 (d, J=4.2 Hz, 3H), 6.93 (d, J=5.0 Hz, 1H), 7.15 (d, J=8.5 Hz, 1H), 7.49 (d, J=8.6 Hz, 1H), 7.99 (d, J=1.5 Hz, 1H), 9.04 (s, 1H), 13.03 (s, 1H); ¹⁹F NMR (376 MHz, DMSO-d₆) δ ppm −60.04 (s, 3F); LCMS (ESI) m/z 323.1 [M+H]⁺.

### Example 6

### Synthesis of N2-(4-fluoro-1H-indazol-6-yl)-N4-methyl-5-(trifluoromethyl)pyrimidine-2,4-diamine

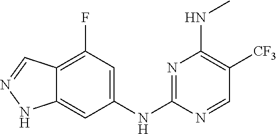 [SMILES: CNc1nc(Nc2cc(F)c3cn[nH]c3c2)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 4-fluoro-1H-indazol-6-amine was used in place of 1H-indazol-6-amine. The crude product was purified by reverse phase preparative HPLC (Gemini® 5 um NX-C18 110 Å, 100×30 mm; mobile phase: [water (10 mM Ammonium Bicarbonate pH-=10.0 v/v)-MeOH]; B %: 50%-100%, 16 min), to provide title compound as a white powder (20.0 mg, 13%). ¹H NMR (400 MHz, DMSO-d₆) δ ppm 3.00 (d, J=4.3 Hz, 3H), 7.30-7.24 (m, 2H), 8.03 (s, 1H), 8.09 (s, 1H), 8.23 (s, 1H), 9.92 (s, 1H), 13.20 (s, 1H); ¹⁹F NMR (376 MHz, DMSO-d₆) δ ppm −6053 (s, 3F), −118.40 (s, 1F); LCMS (ESI) m/z 327.0 [M+H]⁺.

### Example 7

### Synthesis of N2-(3-bromo-1H-indazol-6-yl)-N4-methyl-5-(trifluoromethyl)pyrimidine-2,4-diamine

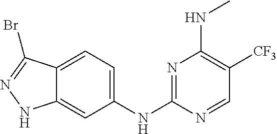 [SMILES: CNc1nc(Nc2ccc3c(Br)n[nH]c3c2)ncc1C(F)(F)F]

### Step 1:

To a mixture of 3-bromo-6-nitro-1H-indazole (0.30 g, 1.20 mmol, 1.00 eq) in EtOH (10.0 mL) and water (21.0 ml) was added NH₄Cl (0.32 g, 6.01 mmol, 5.00 eq) in one portion, followed by the addition of Fe (0.36 g, 6.01 mmol, 5.00 eq) at room temperature. The reaction mixture was heated at 80° C. for 12 h. After cooling to room temperature, the mixture was filtered over celite and washed with 10% MeOH/CH₂Cl₂ (10 mL×2). The filtrate was concentrated under reduced pressure and the residue was then dissolved in EtOAc (20 mL). Water (20 mL) was added, and the mixture was extracted with EtOAc (20 ml×2). The combined organic layers were washed with brine (50 mL), dried over anhydrous MgSO₄, and filtered. The resulting organic layer was concentrated under reduced pressure to provide 3-bromo-1H-indazol-6-amine (Intermediate 7-a) as a off white solid (225 mg, 99%). LCMS (ESI) m/z 212.0 [M+H]⁺. The crude product was used directly in the next step without further purification.

### Step 2

The title compound was made from 2-chloro-N-methyl-5-(trifluoromethyl)pyrimidin-4-amine (Intermediate 1-a) following the procedure as described in Example 1, Step 2, except that 3-bromo-1H-indazol-6-amine (Intermediate 7-a) was used in place of 1H-indazol-6-amine. The crude product was purified by flash chromatography on silica gel column using 100-90% DCM/MeOH as eluent, to provide title compound as a white powder (15.0 mg, 8%). 1H NMR (400 MHz, DMSO-d₆) δ ppm 3.00 (d, J=4.4 Hz, 3H), 7.24 (s, 1H), 7.42-7.41 (m, 2H), 8.23 (s, 1H), 8.39 (s, 1H), 9.94 (s, 1H), 13.20 (s, 1H); ¹⁹F NMR (376 MHz, DMSO-d₆) δ ppm −60.48 (s, 3F); LCMS (ESI) m/z 389.0 [M+H]⁺.

### Example 8

### Synthesis of 6-((4-(methylamino)-5-(trifluoromethyl)pyrimidin-2-yl)amino)-1H-indazole-3-carbonitrile

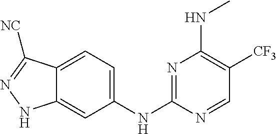 [SMILES: CNc1nc(Nc2ccc3c(C#N)n[nH]c3c2)ncc1C(F)(F)F]

### Step 1

To a solution of 3-Bromo-6-nitroindazole (0.21 mL, 0.83 mmol) in DMF (3.0 mL) was added 60% wt Sodium hydride (49.60 mg, 1.24 mmol) portion-wise over 5 mins at rt. The crude was stirred at rt for 30 mins before the addition of 2-(Trimethylsilyl)ethoxymethyl chloride (0.21 mL, 1.07 mmol) at rt. The crude was stirred at rt for 18 hrs. The crude was quenched with H₂O and diluted in EtOAc. The aq. Layer was extracted with EtOAc (20.0 mL×3). The combined organic layer was washed with H₂O, brine, dried over MgSO₄, filtered, and evaporated. The crude product was purified by flash chromatography on silica gel column using 0-90% EtOAc/Hexane as eluent, to provide 3-bromo-6-nitro-1-((2-(trimethylsilyl)ethoxy)methyl)-1-f-indazole (Intermediate 8-a) as an off white solid (0.30 g, 08%). LCMS doesn't show the exact mass of the desired product. The structure was confirmed using HNMR and it clearly showed trace of the N2 stereoisomers.

### Step 2

To a solution of 3-bromo-6-nitro-1-((2-(trimethylsilyl)ethoxy)methyl)-1H-indazole (Intermediate 8-a) (50.00 mg, 0.13 mmol) in DMF (2.0 mL) in a microwave reaction vessel was added Zinc Cyanide (15.80 mg, 0.13 mmol), and 1,1′-Bis(diphenylphosphino)ferrocene dichloropalladium (II) (10.30 mg, 0.01 mmol). The crude was degassed and sealed with a reaction vessel cap. The crude was heated at 180° C. in a microwave for 30 min. The crude was cooled and quenched with water at rt. The crude was diluted in EtOAc (2.0 mL) and extracted with 3×15.0 mL of EtOAc. The organic layer was dried over Na₂SO₄, filtered, and evaporated. The crude product was purified by flash chromatography on silica gel column using 0-90% EtOAc/Hexane as eluent, to provide 6-nitro-1-((2-(trimethylsilyl)ethoxy)methyl)-H-indazole-3-carbonitrile (intermediate 8-b) as a off white solid (0.30 g, 98%). LCMS doesn't show the exact mass of the desired product. The structure was confirmed using HNMR and it clearly showed trace of the N₂ stereoisomers.

### Step 3

The title compound was made from 6-nitro-1-((2-(trimethylsilyl)ethoxy)methyl)-1H-indazole-3-carbonitrile (Intermediate 8-b) following the procedure as described in Example 7, Step 1 to provide 6-amino-1-((2-(trimethylsilyl)ethoxy)methyl)-1F-indazole-3-carbonitrile (intermediate 8-c) as a white solid (50.0 mg, 92%). LCMS (ESI) m/z 287.1 [M−H]⁻.

### Step 4

The title compound was made from 2-chloro-N-methyl-5-(trifluoromethyl)pyrimidin-4-amine (Intermediate 1-a) following the procedure as described in Example 1, Step 2, except that 6-amino-1-((2-(trimethylsilyl)ethoxy)methyl)-1H-indazole-3-carbonitrile (Intermediate 8-c) was used in place of 1H-indazol-6-amine, to provide crude product of 1-(hydroxymethyl)-6-((4-(methylamino)-5-(trifluoromethyl)pyrimidin-2-yl)amino)-1H-indazole-3-carbonitrile (Intermediate 8-d) as a white powder (30.0 mg, 70%). LCMS (ESI) m/z 334.0 [M+H]⁺.

### Step 5

The crude material 1-(hydroxymethyl)-6-((4-(methylamino)-5-(trifluoromethyl)pyrimidin-2-yl)amino)-1H-indazole-3-carbonitrile (Intermediate 8-d) was dissolved in mixture of MeOH (1.6 mL) and H₂O (0.3 mL). The mixture was stirred at rt for 3 d. The volatiles were removed in vacuo and the residue was purified by reverse phase preparative HPLC (Gemini® 5 um NX-C18 110 Å, 100×30 mm; mobile phase: [water (10 mM Ammonium Formate pH=3.8 v/v)-MeOH]; B %: 50%-100%, 16 min) to provide title compound as a white powder (4.0 mg, 15%). ¹H NMR (400 MHz, DMSO-d₆) δ ppm 3.01 (d, J=4.2 Hz, 3H), 7.25-7.22 (m, 1H), 7.50 (d, J=8.7 Hz, 1H), 7.69 (d, J=8.7 Hz, 1H), 8.23 (s, 1H), 8.53 (s, 1H), 9.95 (s, 1H); ¹⁹F NMR (376 MHz, DMSO-d₆) δ ppm −60.45 (s, 3F); LCMS (ESI) m/z 334.0 [M+H]⁺.

### Example 9

### Synthesis of (6-((4-(methylamino)-5-(trifluaoromethyl)pyrimidin-2-yl)amino)-1H-indazol-3-yl)(morpholino)methanone

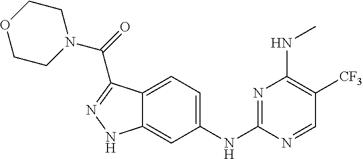 [SMILES: CNc1nc(Nc2ccc3c(C(=O)N4CCOCC4)n[nH]c3c2)ncc1C(F)(F)F]

### Step 1

A solution of potassium permanganate (0.15 g, 0.92 mmol, 1.00 eq) in H₂O (1.0 mL) was added dropwise to a mixture of 6-Nitro-1H-indazole-3-carbaldehyde (0.10 g, 0.51 mmol, 1.00 eq) in acetone (3.0 mL) at room temperature. The reaction mixture was stirred at room temperature for 6 h. Hydrogen peroxide (0.30 mL, 2.56 mmol, 5.00 eq) was added dropwise to neutralize the KMnO₄. The reaction mixture was filtered to remove the KMnO₄ followed by concentration to dryness. The residue was acidified by conc. HCl, filtered, and recrystallized from methanol to provide 6-nitro-1H-indazole-3-carboxylic acid (Intermediate 9-a) as a light-yellow solid (0.11 g, 99%). LCMS (ESI) m/z 206.1 [M−H]⁻.

### Step 2

6-nitro-1H-indazole-3-carboxylic acid (Intermediate 9-a) (50.0 mg, 0.241 mmol), 1-methylimidazole (70.10 uL, 0.85 mmol, 3.50 eq), morpholine (21.30 uL, 0.24 mmol, 1.00 eq) and 1,8-diazabicyclo[5.4.0]undec-7-ene (36.10 uL, 0.24 mmol, 1.00 eq) were dissolved in anhydrous DMF (2.0 mL). N-(Chloro(dimethylamino)methylene)-N-methylmethanaminium hexafluorophosphate(V) (76.00 mg, 0.27 mmol, 1.10 eq) was added to the above solution. The reaction mixture was stirred at it for 1 h followed by quenching with EtOAc (2.0 mL). The resulted mixture was diluted with water and was extracted with EtOAc (15 mL×3). The combined organic extract was dried and concentrated to dryness to provide morpholino(6-nitro-1H-indazol-3-yl)methanone (Intermediate 9-b) as a light-yellow solid (60.0 mg, 90%). LCMS (ESI) m/z 277.1 [M+H]⁺.

### Step 3

The title compound was made from morpholino(6-nitro-1H-indazol-3-yl)methanone (Intermediate 9-b) following the procedure as described in Example 7, Step 1 to provide (6-amino-1H-indazol-3-yl)(morpholino)methanone (Intermediate 9-c) as a white solid (5310 mg, 99%). LCMS (ESI) m/z 247.1 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that (6-amino-1H-indazol-3-yl)(morpholino)methanone (Intermediate 9-c) was used in place of 1H-indazol-6-amine. The crude product was purified by reverse phase preparative HPLC (Gemini® 5 um NX-C18 110 Å, 100×30 mm; mobile phase: [water (10 mM Ammonium Bicarbonate pH=10.0 v/v)-MeOH]; B %: 55%-100%, 16 min), to provide title compound as a white powder (10.0 mg, 10%). ¹H NMR (400 MHz, DMSO-d₆) δ ppm 3.01 (d, J=4.3 Hz, 3H), 3.73-3.59 (m, 8H), 7.22-7.18 (m, 1H), 7.42 (dd, J=8.9, 1.7 Hz, 1H), 7.82 (d, J=8.8 Hz, 1H), 8.22 (s, 1H), 8.37 (s, 1H), 9.86 (s, 1H); ¹⁹F NMR (376 MHz, DMSO-d₆) δ ppm −60.39 (s, 3F); LCMS (ESI) m/z 422.1 [M+H]⁺.

### Example 10

### Synthesis of N²-(3-bromo-1-methyl-1H-indazol-6-yl)-N⁴-methyl-5-(trifluoromethyl) pyrimidine-2,4-diamine

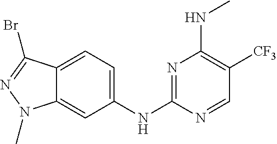 [SMILES: CNc1nc(Nc2ccc3c(Br)nn(C)c3c2)ncc1C(F)(F)F]

### Step 1

To a solution of 3-bromo-6-nitroindazole (0.500 g, 2.07 mmol, 1.00 eq) in DMF (7.0 mL) were added Cs₂CO₃ (0.87 g, 2.69 mmol, 1.30 eq) and MeI (0.15 mL, 2.48 mmol, 1.20 eq). The reaction mixture was stirred at room temperature for 18 h under argon atmosphere. The reaction mixture was poured into water (20 mL) and extracted with EtOAc (20 ml×2). The organic layer was washed with water (25 mL), brine (25 mL), dried over anhydrous MgSO₄, filtered, and concentrated under reduced pressure. The crude product was purified by flash chromatography on silica gel column using 0-30% EtOAc/Hexanes as eluent, to provide 3-bromo-1-methyl-6-nitro-1H-indazole (Intermediate 10-a) as a yellow solid (0.42 g, 79%). ¹H NMR (400 MHz, DMSO-d₆) δ ppm 4.18 (s, 3H), 7.81 (dd, J=8.9, 0.6 Hz, 1H), 8.01 (dd, J=8.9, 1.9 Hz, 1H), 8.80-8.79 (m, 1H).

### Step 2

The title compound was made from 3-bromo-1-methyl-6-nitro-1H-indazole (Intermediate 10-a) following the procedure as described in Example 7, Step 1. The volatiles were removed under reduced pressure to provide 3-bromo-1-methyl-1H-indazol-6-amine (Intermediate 10-b) as a pink solid (62.0 mg, 70%). LCMS (ESI) m/z 228.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 3-bromo-1-methyl-1H-indazol-6-amine (Intermediate 10-b) was used in place of 1H-indazol-6-anine. The crude product was purified by flash chromatography on silica gel column using 0-40% EtOAc/Hexanes as eluent, to provide the title compound as a white solid (90.0 mg, 82%). ¹H NMR (400 MHz, DMSO-d₆) δ ppm 3.04 (d, J=4.2 Hz, 3H), 3.93 (s, 3H), 7.33-7.29 (m, 2H), 7.43 (d, J=8.8 Hz, 1H), 8.23 (s, 1H), 8.54 (s, 1H), 10.03 (s, 1H); ¹⁹F NMR (376 MHz, DMSO-d₆) δ ppm −60.6 (s, 3F); LCMS (ESI) m/z 403.0 [M+H]⁺.

### Example 11

### Synthesis of 1-methyl-6-((4-(methylamino)-5-(trifluoromethyl)pyrimidin-2-yl)amino)-1H-indazole-3-carbonitrile

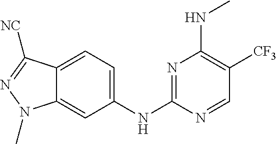 [SMILES: CNc1nc(Nc2ccc3c(C#N)nn(C)c3c2)ncc1C(F)(F)F]

Step 1

The title compound was made from 3-bromo-1-methyl-6-nitro-1H-indazole (Intermediate 10-a) following the procedure as described in Example 8, Step 2 to provide 1-methyl-6-nitro-1H-indazole-3-carbonitrile (Intermediate 11-a) as a white solid (20.0 mg, 52%). ¹H NMR (400 MHz, DMSO-d₆) δ (ppm 4.28 (s, 3H), 8.01 (dd, J=9.0, 0.7 Hz, 1H), 8.22 (dd, J=9.0, 1.9 Hz, 1H), 8.55 (dd, J=1.9, 0.7 Hz, 1H).

### Step 2

The title compound was made from 1-methyl-6-nitro-1H-indazole-3-carbonitrile (Intermediate 11-a) following the procedure as described in Example 7, Step 1 to provide 6-amino-1-methyl-1H-indazole-3-carbonitrile (Intermediate 11-b) as a white solid (12.0 mg, 99%). LCMS (ESI) m/z 173.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 6-amino-1-methyl-1H-indazole-3-carbonitrile (Intermediate 11-b) was used in place of 1H-indazol-6-amine. The crude product was purified by reverse phase preparative HPLC (Gemini® 5 um NX-C18 110 Å, 100×30 mm; mobile phase: [water (10 mM Ammonium Bicarbonate pH=10.0 v/v)-MeOH]; B %: 55%-100%, 16 min), to provide title compound as a white powder (4.0 mg, 12%). ¹H NMR (400 MHz, DMSO-d₆) δ ppm 3.06 (d, J=4.2 Hz, 3H), 4.09 (s, 3H), 7.34 (m, 1H), 7.49 (d, J=8.9 Hz, 1H), 7.75 (d, J=8.9 Hz, 1H), 8.25 (s, 1H), 8.72 (s, 1H), 10.15 (s, 1H); ¹⁹F NMR (376 MHz, DMSO-d₆) δ ppm −60.65 (s, 3H) LCMS (ESI) m/z 348.0 [M+H]⁺.

### Example 12

### Synthesis of N4-methyl-N2-(1-methyl-1H-indazol-6-yl)-5-(trifluoromethyl)pyrimidine-2,4-diamine

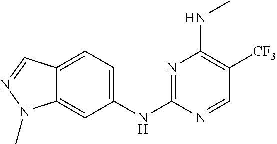 [SMILES: CNc1nc(Nc2ccc3cnn(C)c3c2)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 1-methyl-1H-indazol-6-amine was used in place of 1H-indazol-6-amine. The crude product was purified by flash chromatography on silica gel column using 0-90% EtOAc/Hexane as eluent, to provide title compound as a white powder (30.0 mg, 20%). ¹H NMR (400 MHz, DMSO-d₆) δ ppm 3.05 (d, J=4.0 Hz, 3H), 3.95 (s, 3H), 7.25 (m, 2H), 7.60 (d, J=8.7 Hz, 1H), 7.90 (s, 1H), 8.22 (s, 1H), 8.50 (s, 1H), 9.91 (s, 1H); ¹⁹F NMR (376 MHz, DMSO-d₆) δ ppm −60.43 (s, 3F); LCMS (ESI) m/z 323.0 [M+H]⁺.

### Example 13

### Synthesis of N2-(1,5-dimethyl-1H-indazol-6-yl)-N4-methyl-5-(trifluoromethyl)pyrimidine-2,4-diamine

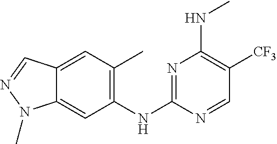 [SMILES: CNc1nc(Nc2cc3c(cnn3C)cc2C)ncc1C(F)(F)F]

### Step 1

Iodomethane (0.39 g, 2.74 mmol, 1.00 eq) was added to a mixture of 5-Methyl-6-nitro-1H-indazole (0.50 g, 2.74 mmol, 1.00 eq) and Cesium carbonate (0.89 g, 2.74 mmol, 1.00 eq) in DMF (7.0 mL) at room temperature. The reaction mixture was stirred at the same temperature for 2 days. The reaction mixture was diluted with water (15 mL) and was extracted with 2×50 mL of EtOAc. Combined organic extracts were dried and concentrated to dryness. The residue was purified by Combi-Flash (25 g Gold SiO2) by eluting from 100% hexanes to 90% EtOAc/hexanes over 25 min to afford 1,5-dimethyl-6-nitro-1H-indazole (Intermediate 13-a) as a white solid (0.3 g, 57%) and 2,5-dimethyl-6-nitro-2H-indazole (Intermediate 13-b) as a white solid (0.1 g, 19%). LCMS (ESI) m/z 192.0 [M+H]⁺.

### Step 2

The title compound was made from 1,5-dimethyl-6-nitro-1H-indazole (Intermediate 13-a) following the procedure as described in Example 7, Step 1 to provide 1,5-dimethyl-6-nitro-1H-indazole (Intermediate 13-c) as a white solid (84.3 mg, 99%). LCMS (ESI) m/z 162.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 1,5-dimethyl-6-nitro-1H-indazole (13-c) was used in place of 1-indazol-6-amine. The crude product was purified by flash chromatography on silica gel column using 0-90% EtOAc/Hexane as eluent, to provide title compound as a white powder (20.0 mg, 13%). ¹H NMR (400 MHz, DMSO-d₆) δ ppm 2.33 (s, 3H), 2.85 (d, J=4.4 Hz, 3H), 3.96 (s, 3H), 7.07 (m, 1H), 7.53 (s, 1H), 7.88 (d, J=1.0 Hz, 1H), 7.94 (s, 1H), 8.13 (s, 1H), 8.81 (s, 1H); ¹⁹F NMR (376 MHz, DMSO-d₆) δ ppm −60.20 (s, 3F); LCMS (ESI) m/z 337.1 [M+H]⁺.

### Example 14

### Synthesis of N2-(7-methoxy-1-methyl-1H-indazol-6-yl)-N4-methyl-5-(trifluoromethyl) pyrimidine-2,4-diamine

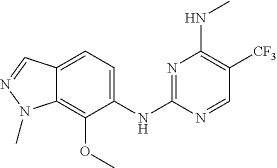 [SMILES: CNc1nc(Nc2ccc3cnn(C)c3c2OC)ncc1C(F)(F)F]

### Step 1

The title compounds were made from 6-bromo-7-methoxy-1H-indazole (Intermediate 4-a) following the procedure as described in Example 10, Step 1. The crude products (3:2 ratio of separable N1:N2 regioisomers) were purified by flash chromatography on silica gel column using 0-40% EtOAc/Hexanes as eluent, to provide title compounds (Intermediate 14-a) (78.0 mg, 49%) and (Intermediate 14-b) (51.0 mg, 32%) as clear oils. LCMS (ESI) m/z 242.9 [M+H]⁺.

### Step 2

The title compound was made from 6-bromo-7-methoxy-methyl-1H-imidazole (Intermediate 14-a) following the procedure as described in Example 4, Step 3. The crude product was purified by flash chromatography on silica gel column using 0-40% EtOAc/Hexanes as eluent, to provide the tert-butyl (7-methoxy-1-methyl-1H-indazol-6-yl)carbamate (Intermediate 14-c) as a yellow solid (0.12 g, 80%) LCMS (ESI) m/z 278.1 [M+H]⁺.

### Step 3

The title compound was made from tert-butyl (7-methoxy-1-methyl-1H-indazol-6-yl)carbamate (Intermediate 14-c) following the procedure as described in Example 4, Step 4. The volatiles were removed under reduced pressure to provide the 7-methoxy-1-methyl-1H-indazol-6-amine (Intermediate 14-d) as a brown oil (0.12 g, crude). LCMS (ESI) m/z 178.1 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 7-methoxy-1-methyl-1H-indazol-6-amine (Intermediate 14-d) was used in place of 1H-indazol-6-amine. The crude product was purified by reverse phase chromatography on a C18 column using 0-30% MeCN/H₂O (0.1% FA) as eluent, to provide the title compound as a white powder (21.0 mg, 14%). ¹H NMR (400 MHz, DMSO-d₆) δ ppm 2.82 (d, J=4.4 Hz, 3H), 3.81 (s, 3H), 4.17 (s, 3H), 7.07-7.04 (m, 1H), 7.42 (d, J=8.6 Hz, 1H), 7.49 (d, J=8.7 Hz, 1H), 7.97 (s, 1H), 8.12 (s, 1H), 8.77 (s, 1H); ¹⁹F NMR (376 MHz, DMSO-d₆) δ ppm −60.3 (s, 3F); LCMS (ESI) m/z 353.3 [M+H]⁺.

### Example 15

### Synthesis of N2-(1,7-dimethyl-1H-indazol-6-yl)-N4-methyl-5-(trifluoromethyl)pyrimidine-2,4-diamine

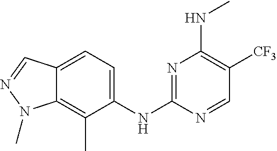 [SMILES: CNc1nc(Nc2ccc3cnn(C)c3c2C)ncc1C(F)(F)F]

### Step 1

The title compound was made from 7-Methyl-6-nitro-1H-indazole following the procedure as described in Example 7, Step 1 to provide 1,7-dimethyl-6-nitro-1H-indazole (Intermediate 15-a) as a white solid (90.0 mg, 28%) and 2,7-dimethyl-6-nitro-2H-indazole (Intermediate 15-b) as a white solid (0.2 g, 70%). LCMS (ESI) m/z 192.0 [M+H]⁺.

### Step 2

The title compound was made from 1,7-dimethyl-6-nitro-1H-indazole (Intermediate 15-a) following the procedure as described in Example 7, Step 1 to provide 1,7-dimethyl-1H-indazol-6-amine (Intermediate 15-c) as a white solid (76.0 mg, 99%). LCMS (ESI) m/z 162.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 1,7-dimethyl-1H-indazol-6-amine (Intermediate 15-c) was used in place of 1H-indazol-6-amine. The crude product was purified by flash chromatography on silica gel column using 0-90% EtOAc/Hexane as eluent, to provide title compound as a white powder (70.0 mg, 44%). ¹H NMR (400 MHz, DMSO-d₆) δ ppm 2.55 (s, 3H), 2.76 (d, J=4.3 Hz, 3H), 4.26 (s, 3H), 6.93 (m, 1H), 7.07 (d, J=8.5 Hz, 1H), 7.46 (d, J=85 Hz, 1H), 7.91 (s, 1H), 8.05 (s, 1H), 9.12 (s, 1H); ¹⁹F NMR (376 MHz, DMSO-d₆) δ ppm −60.04 (s, 3F); LCMS (ESI) m/z 337.1 [M+H]⁺.

### Example 16

### Synthesis of 2-(6-((4-(methylamino)-5-(trifluoromethyl)pyrimidin-2-yl)amino)-1H-indazol-1-yl)acetonitrile

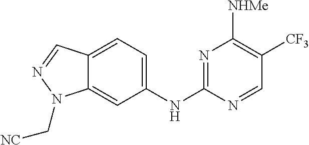 [SMILES: CNc1nc(Nc2ccc3cnn(CC#N)c3c2)ncc1C(F)(F)F]

### Step 1

To a solution of 6-nitroindazole (0.50 g, 3.06 mmol, 1.00 eq) in DMF (10.0 mL) was added Cs₂CO₃ (3.00 g, 9.19 mol, 3.00 eq) and bromoacetonitrile (0.43 mL, 6.13 mmol, 2.00 eq). The reaction mixture was stirred at room temperature for 16 h under argon atmosphere. The reaction mixture was poured into water (20 mL) and extracted with EtOAc (20 ml×2). The organic layer was washed with water (25 mL), brine (25 mL), dried over anhydrous MgSO₄, filtered, and concentrated under reduced pressure. The crude product was purified by flash chromatography on silica gel column using 0-50% EtOAc/Hexanes as eluent, to provide the 2-(6-nitro-1H-indazol-1-yl)acetonitrile (Intermediate 16-a) as a yellow solid (0.24 g, 39%). ¹H NMR (400 MHz, CDCl₃) δ ppm 5.42 (s, 2H), 7.93 (d, J=8.9 Hz, 1H), 8.15 (d, J=8.9 Hz, 1H), 8.23 (s, 1H), 8.47 (s, 1H).

### Step 2

The title compound was made from 2-(6-nitro-1H-indazol-1-yl)acetonitrile (Intermediate 16-a) following the procedure as described in Example 7, Step 1. The volatiles were removed under reduced pressure to provide the 2-(6-amino-1H-indazol-1-yl)acetonitrile (intermediate 16-b) as a red oil (85.0 mg, crude). LCMS (ESI) m/z 173.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 2-(6-amino-1H-indazol-1-yl)acetonitrile (Intermediate 16-b) was used in place of 1H-indazol-6-amine. Trituration of the crude product with acetonitrile provided the title compound as an off-white solid (0.14 g, 82%). ¹H NMR (400 MHz, DMSO-d₆) δ ppm 3.07 (d, J=4.3 Hz, 3H), 5.74 (s, 2H), 7.31 (d, J=8.8 Hz, 1H), 7.74 (m, 2H), 8.12 (s, 1H), 8.32 (s, 1H), 8.47 (s, 1H), 10.39 (s, 1H); ¹⁹F NMR (376 MHz, DMSO-d₆) δ ppm −61.0 (s, 3F); LCMS (ESI) m/z 348.1 [M+H]⁺.

### Example 17

### Synthesis of N4-methyl-N2-(2-methyl-2H-indazol-6-yl)-5-(trifluoromethyl)pyrimidine-2,4-diamine

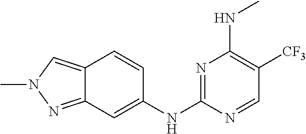 [SMILES: CNc1nc(Nc2ccc3cn(C)nc3c2)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 2-methyl-2H-indazol-6-amine was used in place of 1H-indazol-6-amine. The crude product was purified by flash chromatography on silica gel column using 0-90% EtOAc/Hexane as eluent, to provide title compound as a white powder (60.0 mg, 39%). ¹H NMR (400 MHz, DMSO-d₆) δ ppm 2.99 (d, J=4.2 Hz, 3H), 4.10 (s, 3H), 7.15 (s, 1H), 7.22 (dd, J=9.0, 1.8 Hz, 1H), 7.55 (d, J=9.0 Hz, 1H), 8.19 (d, J=5.0 Hz, 2H), 8.30 (s, 1H), 9.62 (s, 1H), ¹⁹F NMR (376 MHz, DMSO-d₆) δ ppm −60.25 (s, 3F); LCMS (ESI) m/z 323.1 [M+H]⁺.

### Example 18

### Synthesis of N2-(2,5-dimethyl-2H-indazol-6-yl)-N4-methyl-5-(trifluoromethyl)pyrimidine-2,4-diamine

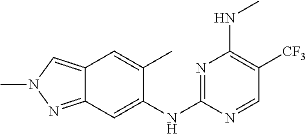 [SMILES: CNc1nc(Nc2cc3nn(C)cc3cc2C)ncc1C(F)(F)F]

### Step 1

The title compound was made from 2,5-dimethyl-6-nitro-22H-indazole (Intermediate 13-b) following the procedure as described in Example 7, Step 1 to provide 2,5-dimethyl-2H-indazol-6-amine (Intermediate 18-a) as a white solid (63.2 mg, 99%). LCMS (ESI) m/z 162.0 [M+H]⁺.

### Step 2

The title compound was made from 2-chloro-N-methyl-5-(trifluoromethyl)pyrimidin-4-amine (Intermediate 1-a) following the procedure as described in Example 1, Step 2, except that 2,5-dimethyl-2H-indazol-6-amine (Intermediate 18-a) was used in place of 1H-indazol-6-amine. The crude product was purified by flash chromatography on silica gel column using 0-90% EtOAc/Hexane as eluent, to provide title compound as a white powder (18.0 mg, 11%). ¹H NMR (400 MHz, DMSO-d₆) δ ppm 2.28 (s, 3H), 2.81 (d, J=4.4 Hz, 3H), 4.11 (s, 3H), 7.00 (m, 1H), 7.47 (s, 1H), 7.75 (s, 1H), 8.09 (s, 1H), 8.16 (s, 1H), 8.69 (s, 1H); ¹⁹F NMR (376 MHz, DMSO-d₆) δ ppm −60.10 (s, 3F); LCMS (ESI) m/z 337.1 [M+H]⁺.

### Example 9

### Synthesis of N2-(2,7-dimethyl-2H-indazol-6-yl)-N4-methyl-5-(trifluoromethyl)pyrimidine-2,4-diamine

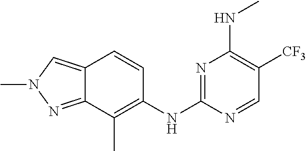 [SMILES: CNc1nc(Nc2ccc3cn(C)nc3c2C)ncc1C(F)(F)F]

### Step 1

The title compound was made from 2,5-dimethyl-6-nitro-2H-indazole (Intermediate 15-b) following the procedure as described in Example 7, Step 1 to provide 2,7-dimethyl-2H-indazol-6-amine (Intermediate 19-a) as a white solid (84.0 mg, 99%). LCMS (ESI) m/z 162.1 [M+H]⁺.

### Step 2

The title compound was made from 2-chloro-N-methyl-5-(trifluoromethyl)pyrimidin-4-amine (Intermediate 1-a) following the procedure as described in Example 1, Step 2, except that 2,7-dimethyl-2H-indazol-6-amine (Intermediate 19-a) was used in place of 1H-indazol-6-amine. The crude product was purified by flash chromatography on silica gel column using 0-90% EtOAc/Hexane as eluent, to provide title compound as a white powder (50.0 mg, 31%). ¹H NMR (400 MHz, DMSO-d₆) δ ppm 2.36 (s, 3H), 2.75 (d, J=4.4 Hz, 3H), 4.14 (s, 3H), 6.90 (m, 1H), 7.05 (d, J=8.8 Hz, 1H), 7.42 (d, J=8.8 Hz, 1H), 8.05 (s, 1H), 8.23 (s, 1H), 8.97 (s, 1H); ¹⁹F NMR (376 MHz, DMSO-d₆) δ ppm −60.10 (s, 3F); LCMS (ESI) m/z 337.1 [M+H]⁺.

### Example 20

### Synthesis of N2-(7-methoxy-2-methyl-2H-indazol-6-yl)-N4-methyl-5-(trifluoromethyl)pyrimidine-2,4-diamine

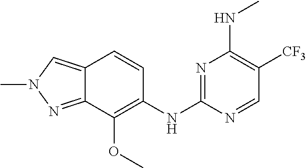 [SMILES: CNc1nc(Nc2ccc3cn(C)nc3c2OC)ncc1C(F)(F)F]

### Step 1

The title compound was made from 6-bromo-7-methoxy-2-methyl-2H-indazole (Intermediate 14-b) following the procedure as described in Example 4, Step 3. The crude product was purified by flash chromatography on silica gel column using 0-30% EtOAc/Hexanes as eluent, to provide tert-butyl (7-methoxy-2-methyl-2H-indazol-6-yl)carbamate (Intermediate 20-a) as a yellow solid (60.0 mg, 67%). LCMS (ESI) m/z 278.1 [M+H]⁺.

### Step 2

The title compound was made from tert-butyl (7-methoxy-2-methyl-2H-indazol-6-yl)carbamate (Intermediate 20-a) following the procedure as described in Example 4, Step 4. The volatiles were removed under reduced pressure to provide 7-methoxy-2-methyl-2H-indazol-6-amine (Intermediate 20-b) as a brown oil (62.8 ng, crude). LCMS (ESI) m/z 178.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 7-methoxy-2-methyl-1H-indazol-6-amine (Intermediate 20-b) was used in place of 1H-indazol-6-amine. The crude product was purified by reverse phase chromatography on a C18 column using 0-30% MeCN/H₂O (0.1% FA) as eluent, to provide the title compound as an off-white powder (38.0 mg, 50%). ¹H NMR (400 MHz, DMSO-d₆) δ ppm 2.84 (d, J=4.4 Hz, 3H), 415 (s, 3H), 4.15 (s, 3H), 7.02-6.99 (m, 1H), 7.31 (d, J=8.9 Hz, 1H), 7.55 (d, J=8.9 Hz, 1H), 8.09 (d, J=1.0 Hz, 1H), 8.30 (s, 1H) 8.43 (s, 1H); ¹⁹F NMR (376 MHz, DMSO-d₆) δ ppm −60.1 (s, 3F); LCMS (ESI) m/z 353.3 [M+H]⁺.

### Example 21

### Synthesis of N2-(1H-indazol-4-yl)-N4-methyl-5-(trifluoromethyl)pyrimidine-2,4-diamine

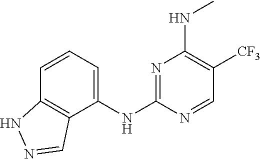 [SMILES: CNc1nc(Nc2cccc3[nH]ncc23)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 1H-indazol-4-amine was used in place of 1H-indazol-6-amine. The crude product was purified by flash chromatography on silica gel column using 0-90% EtOAc/Hexane as eluent, to provide title compound as a white powder (35.0 mg, 24%). ¹H NMR (400 MHz, DMSO-d₆): δH 2.92 (d, J=4.4 Hz, 3H), 7.16 (t, J=8.2 Hz, 2H), 7.27 (t, J=7.9 Hz, 1H), 7.87 (d, J=7.6 Hz, 1H), 8.21 (d, J=1.0 Hz, 1H), 8.40 (s, 1H), 9.76 (s, 1H), 12.96 (s, 1H); ¹⁹F NMR (376 MHz, DMSO-d₆) δ ppm −60.42 (s, 3H) LCMS (ESI) m/z 309.0 [M+H]⁺.

### Example 22

### Synthesis of N2-(5-fluoro-1H-indazol-4-yl)-N4-methyl-5-(trifluoromethyl)pyrimidine-2,4-diamine

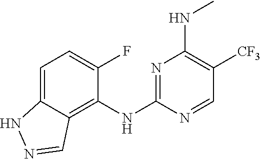 [SMILES: CNc1nc(Nc2c(F)ccc3[nH]ncc23)ncc1C(F)(F)F]

### Step 1

The title compound was made from 2-bromo-3,6-difluorobenzaldehyde following the procedure as described in Example 4, Step 1. The crude product was purified by flash chromatography on silica gel column using 0-25% EtOAc/Hexanes as eluent, to provide the 4-bromo-5-fluoro-1H-indazole (Intermediate 22-a) as a beige solid (0.12 g, 26%). LCMS (ESI) m/z 217.0 [M+H]⁺.

### Step 2

The title compound was made from 4-bromo-5-fluoro-1H-indazole (Intermediate 22-a) following the procedure as described in Example 4, Step 2. The crude product was purified by flash chromatography on silica gel column using 0-10% EtOAc/Hexanes as eluent, to provide 4-bromo-5-fluoro-1-(tetrahydro-2H-pyran-2-yl)-1H-indazole (Intermediate 22-b) as a crystalline solid (0.15 g, 87%). LCMS (ESI) m/z 300.9 [M+H]⁺.

### Step 3

The title compound was made from 4-bromo-5-fluoro-1-(tetrahydro-2H-pyran-2-yl)-1H-indazole (Intermediate 22-b) following the procedure as described in Example 4, Step 3. The crude product was purified by flash chromatography on silica gel column using 0-20% EtOAc/Hexanes as eluent, to provide tert-butyl (5-fluoro-1-(tetrahydro-2H-pyran-2-yl)-1H-indazol-4-yl)carbamate (Intermediate 22-c) as a yellow solid (0.12 g, 81%). LCMS (ESI) m/z 336.1 [M+H]⁺.

### Step 4

The title compound was made from tert-butyl (5-fluoro-1-(tetrahydro-2H-pyran-2-yl)-1H-indazol-4-yl)carbamate (Intermediate 22-c) following the procedure as described in Example 4, Step 4. The volatiles were removed under reduced pressure to provide 5-fluoro-1H-indazol-4-amine (Intermediate 22-d) as a brown oil (0.13 g, crude). LCMS (ESI) m/z 152.1 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 5-fluoro-1H-indazol-4-amine (Intermediate 22-d) was used in place of 1H-indazol-6-amine. The crude product was purified by reverse phase chromatography on a C18 column using 0-30% MeCN/H₂O (0.1% FA) as eluent, to provide the title compound as a pale-yellow solid (11.0 mg, 10%). ¹H NMR (400 MHz, DMSO-d₆) δ ppm 2.70 (d, J=4.3 Hz, 3H), 7.05-7.02 (m, 1H), 7.27 (dd, J=10.5, 8.9 Hz, 1H), 7.36 (dd, J=8.9, 3.6 Hz, 1H), 7.94 (s, 1H), 8.09 (s, 1H), 9.50 (s, 1H), 13.10 (s, 1H); ¹⁹F NMR (376 MHz, DMSO-d₆) δ ppm −60.3 (s, 3F), −73.8 (s, 1F); LCMS (ESI) m/z 327.0 [M+H]⁺.

### Example 23

### Synthesis of N4-methyl-N2-(5-methyl-1H-indazol-4-yl)-5-(trifluoromethyl)pyrimidine-2,4-diamine

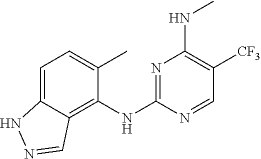 [SMILES: CNc1nc(Nc2c(C)ccc3[nH]ncc23)ncc1C(F)(F)F]

### Step 1

The title compound was made from 2-bromo-6-fluoro-3-methylbenzaldehyde following the procedure as described in Example 4, Step 1. The crude product was purified by flash chromatography on silica gel column using 0-25% EtOAc/Hexanes as eluent, to provide 4-bromo-5-methyl-1H-indazole (Intermediate 23-a) as a beige solid (0.25 g, 27%). LCMS (ESI) m/z 213.0 [M+H]⁺.

### Step 2

The title compound was made from 4-bromo-5-methyl-1H-indazole (Intermediate 23-a) following the procedure as described in Example 4, Step 2. The crude product was purified by flash chromatography on silica gel column using 0-10% EtOAc/Hexanes as eluent, to provide 4-bromo-5-methyl-1-(tetrahydro-2H-pyran-2-yl)-1H-indazole (Intermediate 23-b) as a crystalline solid (0.35 g, 99%). LCMS (ESI) m/z 297.0 [M+H]⁺.

### Step 3

The title compound was made from 4-bromo-5-methyl-1-(tetrahydro-2H-pyran-2-yl)-1H-indazole (Intermediate 23-b) following the procedure as described in Example 4, Step 3. The crude product was purified by flash chromatography on silica gel column using 0-20% EtOAc/Hexanes as eluent, to provide tert-butyl (5-methyl-1-(tetrahydro-2H-pyran-2-yl)-1H-indazol-4-yl)carbamate (Intermediate 23-c) as a yellow solid (0.13 g, 66%). LCMS (ESI) m/z 332.1 [M+H]⁺.

### Step 4

The title compound was made from tert-butyl (5-methyl-1-(tetrahydro-2-H-pyran-2-yl)-1H-indazol-4-yl)carbamate (Intermediate 23-c) following the procedure as described in Example 4, Step 4. The volatiles were removed under reduced pressure to provide 5-methyl-1H-indazol-4-amine (Intermediate 23-d) as a brown oil (0.14 g, crude), LCMS (ESI) m/z 148.1 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 5-methyl-1H-indazol-4-amine (Intermediate 23-d) was used in place of 1H-indazol-6-amine. The crude product was purified by reverse phase chromatography on a C18 column using 0-30% MeCN/H₂O (0.1% FA) as eluent, to provide the title compound as an off-white solid (7.0 mg, 5.8%) ¹H NMR (400 MHz, DMSO-d₆) δ ppm 2.24 (s, 3H), 2.67 (s, 3H), 6.91 (s, 1H), 7.21 (d, J=8.4 Hz, 1H), 7.29 (d, J=8.4 Hz, 1H), 7.73 (s, 1H), 8.04 (s, 1H), 9.24 (s, 1H), 12.86 (s, 1H); ¹⁹F NMR (376 MHz, DMSO-d₆) δ ppm −60.1 (s, 3F); LCMS (ESI) m/z 323.0 [M+H]⁺.

### Example 24

### Synthesis of N4-methyl-N2-(6-methyl-1H-indazol-4-yl)-5-(trifluoromethyl)pyrimidine-2,4-diamine

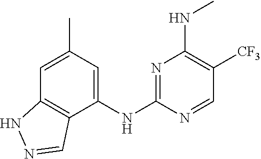 [SMILES: CNc1nc(Nc2cc(C)cc3[nH]ncc23)ncc1C(F)(F)F]

### Step 1

The title compound was made from 2-Bromo-6-fluoro-4-methylbenzaldehyde following the procedure as described in Example 4, Step 1. The crude product was purified by flash chromatography on silica gel column using 0-100% EtOAc/l-exanes as eluent, to provide 4-bromo-6-methyl-1H-indazole (Intermediate 24-a) as a light-yellow fluffy solid (0.16 g, 82%). LCMS (ESI) m/z 213.0 [M+H]⁺.

### Step 2

The title compound was made from 4-bromo-6-methyl-1H-indazole (Intermediate 24-a) following the procedure as described in Example 4, Step 2 to provide 4-bromo-6-methyl-1-(tetrahydro-2H-pyran-2-yl)-H-indazole (Intermediate 24-b) as a white solid (183.0 mg, 82%). LCMS (ESI) m/z 298.0 [M+H]⁺.

### Step 3

The title compound was made from 4-bromo-6-methyl-1-(tetrahydro-2H-pyran-2-yl)-1H-indazole (Intermediate 24-b) following the procedure as described in Example 4, Step 3 to provide (6-methyl-1-(tetrahydro-2H-pyran-2-yl)-1H-indazol-4-yl)carbamate (Intermediate 24-c) as a white solid (170.0 mg, 83%). LCMS (ESI) m/z 332.1 [M+H]⁺.

### Step 4

The title compound was made from (6-methyl-1-(tetrahydro-2H-pyran-2-yl)-1H-indazol-4-yl)carbamate (Intermediate 24-c) following the procedure as described in Example 4, Step 4 to provide 6-methyl-1H-indazol-4-amine (Intermediate 24-d) as a white solid (75.0 mg, 100%). LCMS (ESI) m/z 148.0 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 6-methyl-1H-indazol-4-amine (Intermediate 24-d) was used in place of 1H-indazol-6-amine. The crude product was purified by flash chromatography on silica gel column using 0-90% EtOAc/Hexane as eluent, to provide title compound as a white powder (12.0 mg, 16%). ¹H NMR (400 MHz, DMSO-d₆) δ ppm 2.40 (s, 3H), 2.94 (d, J=4.4 Hz, 3H), 6.94 (d, J=1.3 Hz, 1H), 7.17 (m, 1H), 7.81 (s, 1H), 8.21 (d, J=1.0 Hz, 1H), 8.33 (s, 1H), 9.70 (s, 1H), 12.77 (s, 1H); ¹⁹F NMR (376 MHz, DMSO-d₆) δ ppm −60.33 (s, 3F); LCMS (ESI) m/z 323.1 [M+H]⁺.

### Example 25

### Synthesis of N2-(6-fluoro-1H-indazol-4-yl)-N4-methyl-5-(trifluoromethyl)pyrimidine-2,4-diamine

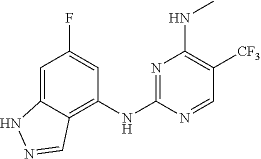 [SMILES: CNc1nc(Nc2cc(F)cc3[nH]ncc23)ncc1C(F)(F)F]

### Step 1

The title compound was made from 6-fluoro-4-nitro-1H-indazole following the procedure as described in Example 7, Step 1. The volatiles were removed under reduced pressure to provide 6-fluoro-H-indazol-4-amine (Intermediate 25-a) as a brown oil (0.16 g, crude). LCMS (ESI) m/z 152.1 [M+H]⁺.

### Step 2

The title compound was made from 2-chloro-N-methyl-5-(trifluoromethyl)pyrimidin-4-amine (Intermediate 1-a) following the procedure as described in Example 1, Step 2, except that 6-fluoro-1H-indazol-4-amine (Intermediate 25-a) was used in place of 1H-indazol-6-amine. The crude product was purified by reverse phase chromatography on a C18 column using 0-40% MeCN/H₂O (0.1% FA) as eluent, to provide the title compound as an off-white powder (4.0 mg, 1.2%). ¹H NMR (400 MHz, DMSO-d₆) δ ppm 2.98 (d, J=4.4 Hz, 3H), 6.90-6.88 (m, 1H), 7.33-7.30 (m, 1H), 8.01 (dd, J=133, 2.1 Hz, 1H), 8.28 (s, 1H), 8.54 (s, 1H), 10.03 (s, 1H), 13.03 (s, 1H); ¹⁹F NMR (376 MHz, DMSO-d₆) δ ppm −60.7 (s, 3F), −113.9 (s, 1F); LCMS (ESI) m/z 327.1 [M+H]⁺.

### Example 26

### Synthesis of N4-methyl-N2-(7-methyl-1H-indazol-4-yl)-5-(trifluoromethyl)pyrimidine-2,4-diamine

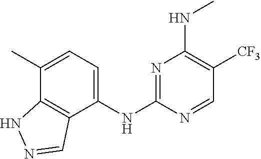 [SMILES: CNc1nc(Nc2ccc(C)c3[nH]ncc23)ncc1C(F)(F)F]

### Step 1

The title compound was made from 6-bromo-2-fluoro-3-methylbenzaldehyde following the procedure as described in Example 4, Step 1. The crude product was purified by flash chromatography on silica gel column using 0-25% EtOAc/Hexanes as eluent, to provide 4-bromo-7-methyl-1H-indazole (Intermediate 26-a) as a white solid (0.12 g, 49%). LCMS (ESI) m/z 211.0 [M+H]⁺.

### Step 2

The title compound was made from 4-bromo-7-methyl-1H-indazole (Intermediate 26-a) following the procedure as described in Example 4, Step 2. The crude product was purified by flash chromatography on silica gel column using 0-10% EtOAc/Hexanes as eluent, to provide 4-bromo-7-methyl-1-(tetrahydro-2H-pyran-2-yl)-1H-indazole (Intermediate 26-b) as a colorless oil (0.16 g, 94%). ¹H NMR (400 MHz, CDCl₃) δ ppm 1.79-1.65 (m, 3H), 2.25-2.04 (m, 3H), 2.57 (d, J=1.1 Hz, 3H), 3.79 (td, J=11.2, 3.0 Hz, 1H), 4.17 (dd, J=11.8, 3.6 Hz, 1H), 5.70 (dd, J=9.6, 2.8 Hz, 1H), 6.90 (dt, J=7.2, 1.2 Hz, 1H), 7.13 (d, J=7.2 Hz, 1H), 8.17 (s, 1H).

### Step 3

The title compound was made from 4-bromo-7-methyl-1-(tetrahydro-2H-pyran-2-yl)-1H-indazole (Intermediate 26-b) following the procedure as described in Example 4, Step 3. The crude product was purified by flash chromatography on silica gel column using 0-40% EtOAc/Hexanes as eluent, to provide tert-butyl (7-methyl-1-(tetrahydro-2H-pyran-2-yl)-1H-indazol-4-yl)carbamate (Intermediate 26-c) as a yellow oil (0.14 g, 83%). LCMS (ESI) m/z 332.2 [M+H]⁺.

### Step 4

The title compound was made from tert-butyl (7-methyl-1-(tetrahydro-2H-pyran-2-yl)-1H-indazol-4-yl)carbamate (Intermediate 26-c) following the procedure as described in Example 4, Step 4. The volatiles were removed under reduced pressure to provide 7-methyl-1H-indazol-4-amine (Intermediate 26-d) as a brown oil (65 mg. crude). LCMS (ESI) m/z 148.1 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 7-methyl-1H-indazol-4-amine (Intermediate 26-d) was used in place of 1H-indazol-6-amine. The crude product was purified by reverse phase chromatography on a C18 column using 0-40% MeCN/H₂O (0.1% FA) as eluent, to provide the title compound as a white solid (6.0 mg, 11%). ¹H NMR (400 MHz, DMSO-d₆) δ ppm 2.45 (s, 3H), 2.90 (d, J=4.3 Hz, 3H), 7.03 (d, J=7.7 Hz, 1H), 7.11 (d, J, 5.1 Hz, 1H), 7.70 (d, J=7.6 Hz, 1H), 8.18 (s, 1H), 8.35 (d, J=1.4 Hz, 1H), 9.63 (s, 1H), 13.01 (s, 1H); ¹⁹F NMR (376 MHz, DMSO-d₆) δ ppm −60.3 (s, 3F); LCMS (ESI) m/z 323.2 [M+H]⁺.

### Example 27

### Synthesis of N2-(7-fluoro-1H-indazol-4-yl)-N4-methyl-5-(trifluoromethyl)pyrimidine-2,4-diamine

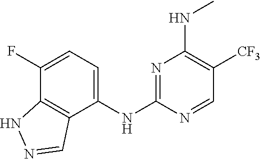 [SMILES: CNc1nc(Nc2ccc(F)c3[nH]ncc23)ncc1C(F)(F)F]

### Step 1

The title compound was made from 4-Bromo-7-fluoro-1H-indazole following the procedure as described in Example 4, Step 2 to provide 4-bromo-7-fluoro-1-(tetrahydro-2H-pyran-2-yl)-1H-indazole (Intermediate 27-a) as a white solid (198.0 mg, 71%). ¹H NMR (400 MHz, CHCl₃-d): δ ppm 1.79-1.59 (m, 3H), 2.20-2.08 (m, 2H), 2.63-2.57 (m, 1H), 3.79-3.73 (m, 1H), 4.09-4.05 (m, 1H), 5.88 (dd, J=9.9, 2.5 Hz, 1H), 6.97 (dd, J=11.4, 8.1 Hz, 1H), 7.22 (dd, J=8.1, 3.5 Hz, 1H), 8.06 (d, J=1.9 Hz, 1H). LCMS (ESI) m/z 216.9 [M+H]⁺.

### Step 2

The title compound was made from 4-bromo-7-fluoro-1-(tetrahydro-2H-pyran-2-yl)-1H-indazole (Intermediate 27-a) following the procedure as described in Example 4, Step 3 to provide tert-butyl (7-fluoro-1-(tetrahydro-2H-pyran-2-yl)-1H-indazol-4-yl)carbamate (Intermediate 27-b) as a white solid (162.0 mug, 73%). LCMS (ESI) m/z 336.1 [M+H]⁺.

### Step 3

The title compound was made from tert-butyl (7-fluoro-1-(tetrahydro-2H-pyran-2-yl)-1H-indazol-4-yl)carbamate (Intermediate 27-b) following the procedure as described in Example 4, Step 4 to provide 7-fluoro-1H-indazol-4-amine (Intermediate 27-c) as a white solid (73.0 mg, 100%). LCMS (ESI) m/z 152.0 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 7-fluoro-1H-indazol-4-amine (Intermediate 27-c) was used in place of 1H-indazol-6-amine. The crude product was purified by flash chromatography on silica gel column using 0-90% EtOAc/Hexane as eluent, to provide title compound as a white powder (50.0 mg, 32%)¹H NMR (400 MHz, DMSO-d₆) δ ppm 2.89 (d, J=4.4 Hz, 3H), 7.16-7.10 (m, 2H), 7.72-7.69 (m, 1H), 8.19 (d, J=0.9 Hz, 1H), 8.44 (dd, J=3.3, 1.6 Hz, 1H), 9.75 (s, 1H), 13.51 (s, 1H); ¹⁹F NMR (376 MHz, DMSO-d₆) δ ppm −60.38 (s, 3F), −140.00 (s, 1F); LCMS (ESI) m/z 323.1 [M+H]⁺.

### Example 28

### Synthesis of N2-(7-methoxy-1H-indazol-4-yl)-N4-methyl-5-(trifluoromethyl)pyrimidine-2,4-diamine

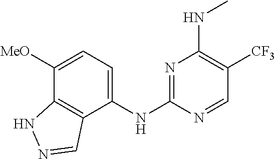 [SMILES: CNc1nc(Nc2ccc(OC)c3[nH]ncc23)ncc1C(F)(F)F]

### Step 1

The title compound was made from 6-Bromo-2-fluoro-3-methoxybenzaldehyde following the procedure as described in Example 4, Step 1. The crude product was purified by flash chromatography on silica gel column using 0-100% EtOAc/Hexanes as eluent, to provide 4-bromo-7-methoxy-1H-indazole (Intermediate 28-a) as a light-yellow solid (0.19 g, 97%). LCMS (ESI) m/z 229.0 [M+H]⁺.

### Step 2

The title compound was made from 4-bromo-7-methoxy-1-indazole (Intermediate 28-a) following the procedure as described in Example 4, Step 2 to provide 4-bromo-7-methoxy-1-(tetrahydro-2H-pyran-2-yl)-1H-indazole (Intermediate 28-b) as a white solid (131.0 mg, 52%). LCMS (ESI) m/z 312.9 [M+H]⁺.

### Step 3

The title compound was made from 4-bromo-7-methoxy-1-(tetrahydro-2H-pyran-2-yl)-1H-indazole (Intermediate 28-b) following the procedure as described in Example 4, Step 3 to provide tert-butyl (7-methoxy-1-(tetrahydro-2H-pyran-2-yl)-1H-indazol-4-yl)carbamate (Intermediate 28-c) as a white solid (128.0 mg, 88%). LCMS (ESI) m/z 348.1 [M+H]⁺.

### Step 4

The title compound was made from tert-butyl (7-methoxy-1-(tetrahydro-2H-pyran-2-yl)-1H-indazol-4-yl)carbamate (intermediate 28-c) following the procedure as described in Example 4, Step 4 to provide 7-methoxy-1H-indazol-4-amine (intermediate 28-d) as a white solid (60.0 mg, 100%). LCMS (ESI) m/z 164.0 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 7-methoxy-1H-indazol-4-amine (Intermediate 28-d) was used in place of 1H-indazol-6-amine. The crude product was purified by flash chromatography on silica gel column using 0-90% EtOAc/Hexane as eluent, to provide title compound as a white powder (8.0 mg, 10%). ¹H NMR (400 MHz, DMSO-d₆) δ ppm 2.87 (d, J=4.4 Hz, 3H), 3.92 (s, 3H), 6.76 (d, J=8.3 Hz, 1H), 7.06 (s, 1H), 7.51 (d, 1H), 8.15 (s, 1H), 8.25 (s, 1H), 9.52 (s, 1H), 13.19 (s, 1H); ¹⁹F NMR (376 MHz, DMSO-d₆) δ ppm −60.15 (s, 3F); LCMS (ESI) m/z 339.0 [M+H]⁺.

### Example 29

### Synthesis of N4-methyl-N2-(1-methyl-1H-indazol-4-yl)-5-(trifluoromethyl)pyrimidine-2,4-diamine

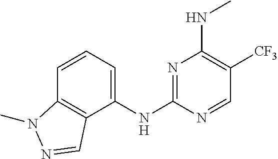 [SMILES: CNc1nc(Nc2cccc3c2cnn3C)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 1-methyl-1H-indazol-4-amine was used in place of 1H-indazol-6-amine. The crude product was purified by flash chromatography on silica gel column using 0-90% EtOAc/Hexane as eluent, to provide title compound as a white powder (35.0 mg, 24%). ¹H NMR (400 MHz, DMSO-d₆) δ ppm 2.93 (d, J=4.4 Hz, 3H), 4.00 (s, 3H), 7.33-7.18 (m, 3H), 7.91 (d, J=7.6 Hz, 1H), 8.22 (s, 1H), 8.38 (d, J=0.9 Hz, 1H), 9.78 (s, 1H); ¹⁹F NMR (376 MHz, DMSO-d₆) δ ppm −60.42 (s, 3F); LCMS (ESI) m/z 323.1 [M+H]⁺.

### Example 30

### Synthesis of 2-(4-((4-(methylamino)-5-(trifluoromethyl)pyrimidin-2-yl)amino)-1H-indazol-1-yl)acetonitrile

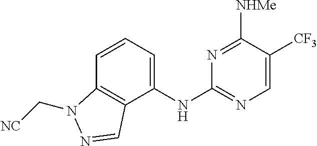 [SMILES: CNc1nc(Nc2cccc3c2cnn3CC#N)ncc1C(F)(F)F]

### Step 1

The title compound was made from 4-nitro-1H-indazole following the procedure as described in Example 16, step 1. The crude product was purified by flash chromatography on silica gel column using 0-50% EtOAc/Hexanes as eluent, to provide the 2-(4-nitro-1H-indazol-1-yl)acetonitrile (Intermediate 30-a) as a beige solid (0.36 g, 58%). ¹H NMR (400 MHz, DMSO-d₆) δ ppm 5.97 (s, 2H), 7.77 (dd, J=8.5, 7.7 Hz, 1H), 8.24 (dd, J=7.7, 0.6 Hz, 1H), 8.35 (dt, J=8.5, 0.8 Hz, 1H), 8.66 (d, J=1.0 Hz, 1H).

### Step 2

The title compound was made from 2-(4-nitro-1H-indazol-1-yl)acetonitrile (Intermediate 30-a) following the procedure as described in Example 7, Step 1 The volatiles were removed under reduced pressure to provide the 2-(4-amino-1H-indazol-1-yl)acetonitrile (Intermediate 30-b) as an orange oil (0.12 g, crude). LCMS (ESI) m/z 173.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 2-(4-amino-1H-indazol-1-yl)acetonitrile (intermediate 30-b) was used in place of 1H-indazol-6-amine. The crude product was purified by reverse phase preparative HPLC (Gemini® 5 um NX-C18 110 Å, 100×30 mm; mobile phase: [water (10 mM Ammonium Bicarbonate pH=10.0 v/v)-MeCN]; B %: 35%-100%, 16 min), to provide the title compound as a white solid (29.0 mg, 13%) ¹H NMR (400 MHz, DMSO-d₆) δ ppm 2.92 (d, J=4.2 Hz, 3H), 5.76 (s, 2H), 7.20 (s, 1H), 7.45-7.37 (m, 2H), 7.97 (d, J=7.5 Hz, 1H), 8.22 (s, 1H), 8.54 (s, 1H), 9.89 (s, 1H); ¹⁹F NMR (376 MHz, DMSO-d₆) δ ppm −60.5 (s, 3F); LCMS (ESI) m/z 348.1 [M+H]⁺.

### Example 31

### Synthesis of 2-methyl-2-(4-((4-(methylamino)-5-(trifluoromethyl)pyrimidin-2-yl)amino)-1H-indazol-1-yl)propanenitrile

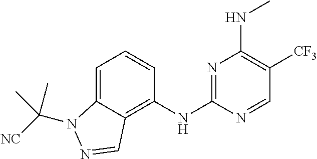 [SMILES: CNc1nc(Nc2cccc3c2cnn3C(C)(C)C#N)ncc1C(F)(F)F]

### Step 1

To a solution of 4-nitro-1H-indazole (1.00 g, 6.13 mmol, 1.00 eq) in DMF (20.0 mL) was added Cs₂CO₃ (3.00 g, 9.19 mmol, 1.50 eq) and 2-bromo-2-methylpropionamide (1.53 g, 9.19 mmol, 1.50 eq). The reaction mixture was warmed to 100° C. and stirred for 3 h under argon atmosphere.

After cooling to room temperature, the reaction mixture was poured into water (50 mL) and extracted with EtOAc (50 ml×3). The combined organic layers were washed with brine (20 mL×3), dried over anhydrous MgSO₄, filtered, and concentrated under reduced pressure. The crude products (1:1 ratio of separable N1:N2 regioisomers) were purified by flash chromatography on silica gel column using 0-30% EtOAc/CH₂Cl₂ to provide title compounds (Intermediate 31-a) (530.0 mg, 35%) and (Intermediate 31-b) (570.0 mg, 37%) as yellow solids. LCMS (ESI) m/z 249.1 [M+H]⁺.

### Step 2

A solution of 2-methyl-2-(4-nitro-1H-indazol-1-yl)propenamide (Intermediate 31-a) (0.52 g, 2.11 mmol, 1.00 eq) in POCl₃ (6.00 mL, 64.40 mmol, 30.40 eq) was warmed to 90° C. and stirred for 1 h. The reaction mixture was cooled to room temperature and was then concentrated under reduced pressure. The residue was poured into ice-water (w/w=1/1; 25 mL) and stirred for 30 min. The aqueous layer was adjusted to pH=7 with a saturated solution of NaHCO₃ (25 mL) and extracted with EtOAc (50 ml×3). The combined organic layers were washed with brine (20 mL 2), dried over anhydrous MgSO₄, filtered, and concentrated under reduced pressure to provide 2-methyl-2-(4-nitro-1H-indazol-1-yl)propanenitrile (Intermediate 31-c) as a pale-yellow solid (0.49 g, crude). ¹HNMR (400 MHz, DMSO-d₆) δ ppm 2.16 (s, 6H), 7.79 (t, J=8.2 Hz, 1H), 8.28 (d, J=7.7 Hz, 1H), 8.48 (d, J=8.6 Hz, 1H), 8.67 (d, 1.9 Hz, 1H).

### Step 3

The title compound was made from 2-methyl-2-(4-nitro-1H-indazol-1-yl)propanenitrile (Intermediate 31-c) following the procedure as described in Example 7, Step 1. The volatiles were removed under reduced pressure to provide 2-(4-amino-1H-indazol-1-yl)-2-methylpropanenitrile (Intermediate 31-d) as a yellow solid (0.43 g, crude). LCMS (ESI) m/z 201.2 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 2-(4-amino-1H-indazol-1-yl)-2-methylpropanenitrile (Intermediate 31-d) was used in place of 1H-indazol-6-amine. The crude product was purified by flash chromatography on silica gel column using 0-50% EtOAc/Hexanes as eluent, to provide the title compound as a white solid (107.0 mg, 67%). ¹H NMR (400 MHz, DMSO-d₆) δ ppm 2.08 (s, 6H), 2.94 (d, J=4.4 Hz, 3H), 7.23-7.21 (m, 1H), 7.49-7.42 (m, 2H), 8.02 (d, J=7.3 Hz, 1H), 8.23 (d, J=1.0 Hz, 1H), 8.55 (d, J=0.8 Hz, 1H), 9.89 (s, 1H); ¹⁹F NMR (376 MHz, DMSO-d₆) δ ppm −60.5 (s, 3F); LCMS (ESI) m/z 376.1 [M+H]⁺.

### Example 32

### Synthesis of (4-((4-(methylamino)-5-(trifluoromethyl)pyrimidin-2-yl)amino)-1H-indazol-1-yl)(morpholino)methanone

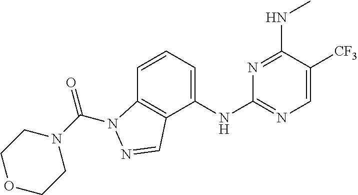 [SMILES: CNc1nc(Nc2cccc3c2cnn3C(=O)N2CCOCC2)ncc1C(F)(F)F]

### Step 1

The title compounds were made from 4-nitro-1H-indazole following the procedure as described in Example 16, step 1, except that 4-morpholinylcarbonyl chloride was used in place of bromoacetonitrile. The crude products (3:1 ratio of inseparable N1:N2 regioisomers) were purified by flash chromatography on silica gel column using 0-10% EtOAc/CH₂Cl₂ to provide a mixture of morpholino(4-nitro-1H-indazol-1-yl)methanone (Intermediate 32-a) and morpholino(4-nitro-2H-indazol-2-yl)methanone (Intermediate 32-b) as a yellow solid (0.62 g, 73%, 4:1 ratio of inseparable N1:N2 regioisomers).

### Step 2

The title compounds were made from morpholino(4-nitro-1H-indazol-1-yl)methanone (Intermediate 32-a) and morpholino(4-nitro-2H-indazol-2-yl)methanone (Intermediate 32-b) following the procedure as described in Example 7, Step 1. The crude products (4:1 ratio of inseparable N1:N2 regioisomers) were purified by flash chromatography on silica gel column using 0-70% EtOAc/Hexanes to provide a mixture of (4-amino-1H-indazol-1-yl)(morpholino)methanone (Intermediate 32-c) and (4-amino-2H-indazol-2-yl)(morpholino)methanone (Intermediate 32-d) as an orange oil (0.10 g, 82%, 4:1 ratio of inseparable N1:N2 regioisomers). LCMS (ESI) m/z 247.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that a mixture of (4-amino-1H-indazol-1-yl)(morpholino)methanone (Intermediate 32-c) and (4-amino-2H-indazol-2-yl)(morpholino)methanone (Intermediate 32-d) was used in place of 1H-indazol-6-amine. The crude product was purified by flash chromatography on silica gel column using 0-10% MeOH/CH₂Cl₂ as eluent, to provide the title compound as a white solid (36.0 mg, %) ¹H NMR (4009 MHz, DMSO-d₆) δ ppm 2.91 (d, J=4.3 Hz, 3H), 3.71-3.70 (m, 8H), 7.21 (d, J=5.1 Hz, 1H), 7.45 (t, J=8.1 Hz, 1H), 7.57 (d, J=8.3 Hz, 1H), 8.01 (d, J=7.8 Hz, 1H), 8.22 (s, 1H), 8.67 (s, 1H), 9.95 (s, 1H); ¹⁹F NMR (376 MHz, DMSO-d₆) δ ppm −60.5 (s, 3F); LCMS (ESI) m/z 422.1 [M+H]⁺.

### Example 33

### Synthesis of N2-(1-(cyclopropylsulfonyl)-1H-indazol-4-yl)-N4-methyl-5-(trifluoromethyl)pyrimidine-2,4-diamine

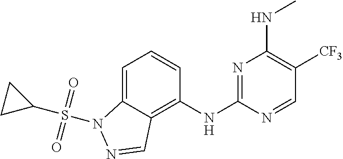 [SMILES: CNc1nc(Nc2cccc3c2cnn3S(=O)(=O)C2CC2)ncc1C(F)(F)F]

### Step 1

To a stirring solution of 4-nitro-1H-indazole (0.25 g, 1.53 mmol, 1.00 eq) in DMF (10.0 mL) cooled to 0° C., was slowly added NaH (91.90 mg, 2.30 mmol, 1.50 eq) (60% dispersion in mineral oil). After stirring for 15 min at 0° C., cyclopropanesulfonyl chloride (0.32 g, 230 mmol, 1.50 eq) was added and the reaction mixture was warmed to room temperature and stirred for 18 h. Water (20 mL) was added, and the mixture was extracted with EtOAc (50 ml×3). The combined organic layers were washed with brine (20 mL×3), dried over anhydrous MgSO₄, filtered, and concentrated under reduced pressure. The crude products (˜1:1 ratio of separable N1:N2 regioisomers) were purified by flash chromatography on silica gel column using 0-50% EtOAc/Hexanes to provide title compounds 1-(cyclopropylsulfonyl)-4-nitro-1H-indazole (Intermediate 33-a) (0.22 g, 53%) and 2-(cyclopropylsulfonyl)-4-nitro-2H-indazole (Intermediate 33-b) (0.11 g, 28%) as yellow solids. LCMS (ESI) m/z 268.0 [M+H]⁺.

### Step 2

To a stirring solution of 1-(cyclopropylsulfonyl)-4-nitro-1H-indazole (intermediate 33-a) (0.10 g, 0.37 mmol, 1.00 eq) in THF (5.00 mL), was added palladium (10 wt. %) on carbon (39.80 mg, 0.04 mmol, 0.10 eq). The reaction mixture was degassed under reduced pressure with backflow of hydrogen (3×) and stirred for 2 h under standard hydrogen atmosphere. The reaction mixture was filtered over Celite and concentrated under reduced pressure to provide 1-(cyclopropylsulfonyl)-1H-indazol-4-amine (Intermediate 33-c) as a pale-yellow solid (88.0 mg, crude). LCMS (ESI) m/z 238.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 1 (cyclopropylsulfonyl)-1H-indazol-4-amine (Intermediate 33-c) was used in place of 1H-indazol-6-amine. The crude product was purified by flash chromatography on silica gel column using 0-50% EtOAc/Hexanes as eluent, to provide the title compound as a white solid (0.14 g, 90%). ¹H NMR (400 MHz, DMSO-d₆) δ ppm 1.1-1.07 (m, 2H), 1.23-1.20 (m, 2H), 2.90 (d, J=4.4 Hz, 3H), 3.06-3.02 (m, 1H), 7.24 (d, J=5.0 Hz, 1H), 7.57-7.53 (m, 1H), 7.61-7.59 (m, 1H), 8.08 (d, J=7.5 Hz, 1H), 8.24 (d, J=1.0 Hz, 1H), 8.88 (d, J=0.8 Hz, 1H), 10.05 (s, 1H); ¹⁹F NMR (376 MHz, DMSO-d₆) δ ppm −60.6 (s, 3F); LCMS (ESI) m/z 413.1 [M+H]⁺.

### Example 34

### Synthesis of N4-methyl-N2-(2-methyl-2H-indazol-4-yl)-5-(trifluoromethyl)pyrimidine-2,4-diamine

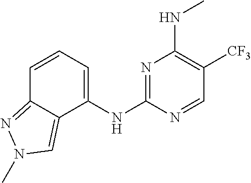 [SMILES: CNc1nc(Nc2cccc3nn(C)cc23)ncc1C(F)(F)F]

### Step 1

The title compounds were made from 4-nitro-1H-indazole following the procedure as described in Example 10, Step 1. The crude products (3:2 ratio of separable N1:N2 regioisomers) were purified by flash chromatography on silica gel column using 60-90% CH₂Cl₂/Hexanes as eluent, to provide 1-methyl-4-nitro-1H-indazole (Intermediate 34-a) (0.63 g, 59%) and 2-methyl-4-nitro-2H-indazole (Intermediate 34-b) (035 g, 32%) as orange solids. LCMS (ESI) m/z 178.1 [M+H]⁺.

### Step 2

The title compound was made from 2-methyl-4-nitro-2H-indazole (Intermediate 34-b) following the procedure as described in Example 7, Step 1. The volatiles were removed under reduced pressure to provide 2-methyl-2H-indazol-4-amine (Intermediate 34-c) as a brown oil (0.13 g, crude). LCMS (ESI) m/z 148.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 2-methyl-2H-indazol-4-amine (Intermediate 34-c) was used in place of 1H-indazol-6-amine. The crude product was purified by reverse phase chromatography on a C18 column using 0-20% MeCN/H₂O (0.1% FA) as eluent, to provide the title compound as a white powder (0.14 g, 48%). ¹H NMR (400 MHz, DMSO-d₆) δ ppm 2.92 (d, J=4.4 Hz, 3H), 4.14 (s, 3H), 7.22-7.13 (m, 3H), 7.74 (d, J=7.2 Hz, 1H), 8.20 (s, 1H), 8.58 (s, 1H), 9.59 (s, 1H); ¹⁹F NMR (376 MHz, DMSO-d₆) δ ppm −60.4 (s, 1F); LCMS (ESI) m/z 323.2 [M+H]⁺.

### Example 35

### Synthesis of N2-(7-methoxy-2-methyl-2H-indazol-4-yl)-N4-methyl-5-(trifluoromethyl) pyrimidine-2,4-diamine

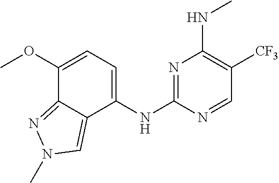 [SMILES: CNc1nc(Nc2ccc(OC)c3nn(C)cc23)ncc1C(F)(F)F]

### Step 1

The title compound was made from 6-bromo-2-fluoro-3-methoxybenzaldehyde following the procedure as described in Example 4, Step 1. The crude product was purified by flash chromatography on silica gel column using 0-90% EtOAc/Hexanes as eluent, to provide 4-bromo-7-methoxy-1H-indazole (Intermediate 35-a) as a white solid (0.37 g, 77%) LCMS (ESI) m/z 229.0 [M+H]⁺.

### Step 2

The title compounds were made from 4-bromo-7-methoxy-1H-indazole (Intermediate 35-a) following the procedure as described in Example 10, Step 1. The crude products (3:1 ratio of separable N1:N2 regioisomers) were purified by flash chromatography on silica gel column using 0-60% EtOAc/Hexanes as eluent, to provide 4-bromo-7-methoxy-1-methyl-1H-indazole (Intermediate 35-b) (0.23 g, 60%) and 4-bromo-7-methoxy-2-methyl-2H-indazole (Intermediate 35-c) (65.0 mug, 17%) as off-white solids. LCMS (ESI) m/z 243.0 [M+H]⁺.

### Step 3

The title compound was made from 4-bromo-7-methoxy-2-methyl-2H-indazole (Intermediate 35-c) following the procedure as described in Example 4, Step 3. The crude product was purified by flash chromatography on silica gel column using 0-20% EtOAc/Hexanes as eluent, to provide tert-butyl (7-methoxy-2-methyl-21H-indazol-4-yl)carbamate (Intermediate 35-d) as a yellow foam (31.0 mg, 45%). LCMS (ESI) m/z 278.2 [M+H]⁺.

### Step 4

The title compound was made from tert-butyl (7-methoxy-2-methyl-2H-indazol-4-yl)carbamate (Intermediate 35-d) following the procedure as described in Example 4, Step 4. The volatiles were removed under reduced pressure to provide 7-methoxy-2-methyl-2H-indazol-4-amine (Intermediate 35-e) as a brown oil (32.4 mg, crude) LCMS (ESI) m/z 178.1 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 7-methoxy-2-methyl-2H-indazol-4-amine (Intermediate 35-e) was used in place of 1H-indazol-6-amine. The crude product was purified by reverse phase chromatography on a C18 column using 0-30% MeCN/H₂O (0.1% FA) as eluent, to provide the title compound as a pale-beige solid (27.0 mag, 69%). ¹H NMR (400 MHz, DMSO-d₆) δ ppm 2.86 (d, J=4.4 Hz, 3H), 3.87 (s, 3H), 4.11 (s, 3H), 6.54 (d, J=8.1 Hz, 1H), 7.02-7.05 (m, 1H), 7.43-7.45 (m, 1H), 8.13 (s, 1H), 8.43 (s, 1H), 9.36 (s, 1H); ¹⁹F NMR (376 MHz, DMSO-d₆) δ ppm −60.1 (s, 3F); LCMS (ESI) m/z 353.0 [M+H]⁺.

### Example 36

### Synthesis of N2-(1,5-dimethyl-1H-indazol-4-yl)-N4-methyl-5-(trifluoromethyl)pyrimidine-2,4-diamine

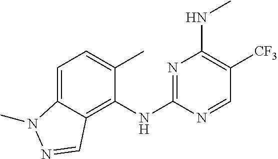 [SMILES: CNc1nc(Nc2c(C)ccc3c2cnn3C)ncc1C(F)(F)F]

### Step 1

The title compounds were made from 4-bromo-5-methyl-1H-indazole following the procedure as described in Example 10, Step 1. The crude products (3:2 ratio of separable N1:N2 regioisomers) were purified by flash chromatography on silica gel column using 0-30% EtOAc/Hexanes as eluent, to provide 4-bromo-1,5-dimethyl-1H-indazole (Intermediate 36-a) (0.72 g, 68%) and 4-bromo-2,5-dimethyl-2H-indazole (Intermediate 36-b) (0.32 g, 30%) as colorless oils. LCMS (ESI) m/z 227.0 [M+H]⁺.

### Step 2

The title compound was made from 4-bromo-1,5-dimethyl-1H-indazole (Intermediate 36-a) following the procedure as described in Example 4, Step 3. The crude product was purified by flash chromatography on silica gel column using 0-50% EtOAc/Hexanes as eluent, to provide the tert-butyl (1,5-dimethyl-1H-indazol-4-yl)carbamate (Intermediate 36-c) as a yellow solid (0.37 g, 99%). LCMS (ESI) m/z 262.2 [M+H]⁺.

### Step 3

The title compound was made from tert-butyl (1,5-dimethyl-1H-indazol-4-yl)carbamate (Intermediate 36-c) following the procedure as described in Example 4, Step 4. The volatiles were removed under reduced pressure to provide 1,5-dimethyl-1H-indazol-4-amine (Intermediate 36-d) as a brown oil (0.36 g, crude). LCMS (ESI) m/z 162.1 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 1,5-dimethyl-1H-indazol-4-amine (Intermediate 36-d) was used in place of 1H-indazol-6-amine. The crude product was purified by reverse phase chromatography on a C18 column using 0-30% MeCN/H₂O (0.1% FA) as eluent, to provide the title compound as an off-white solid (0.18 g, 38%). ¹H NMR (400 MHz, DMSO-d₆) δ ppm 2.25 (s, 3H), 2.68 (s, 3H), 4.00 (s, 3H), 6.87-6.90 (n, 1H), 7.26 (d, J=8.5 Hz, 1H), 7.38 (d, J=8.5 Hz, 1H), 7.69 (d, J=0.9 Hz, 1H), 8.03 (s, 1H), 9.23 (s, 1H); ¹⁹F NMR (376 MHz, DMSO-d₆) δ ppm −60.0 (s, 3H); LCMS (ESI) m/z 337.2 [M+H]⁺.

### Example 37

### Synthesis of N2-(5-methoxy-1-methyl-1H-indazol-4-yl)-N4-methyl-5-(trifluoromethyl) pyrimidine-2,4-diamine

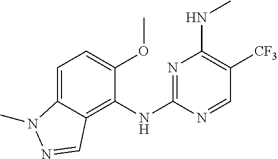 [SMILES: CNc1nc(Nc2c(OC)ccc3c2cnn3C)ncc1C(F)(F)F]

### Step 1

The title compound was made from 2-bromo-6-fluoro-3-methoxybenzaldehyde following the procedure as described in Example 4, Step 1. The crude product was purified by flash chromatography on silica gel column using 0-50% EtOAc/Hexanes as eluent, to provide 4-bromo-5-methoxy-1H-indazole (Intermediate 37-a) as a pale-yellow solid (0.14 g, 14%). LCMS (ESI) m/z 229.0 [M+H]⁺.

### Step 2

The title compounds were made from 4-bromo-5-methoxy-1H-indazole (Intermediate 37-a) following the procedure as described in Example 10, Step 1. The crude products (3:2 ratio of separable N1:N2 regioisomers) were purified by flash chromatography on silica gel column using 0-30% EtOAc/Hexanes as eluent, to provide 4-bromo-5-methoxy-1-methyl-1H-indazole (Intermediate 37-b) (85.3 mg, 60%) and 4-bromo-5-methoxy-2-methyl-2H-indazole (Intermediate 37-c) (37.0 mg, 26%) as white solids. LCMS (ESI) m/z 243.1 [M+H]⁺.

### Step 3

The title compound was made from 4-bromo-5-methoxy-1-methyl-1H-indazole (Intermediate 37-b) following the procedure as described in Example 4, Step 3. The crude product was purified by flash chromatography on silica gel column using 0-50% EtOAc/Hexanes as eluent, to provide tert-butyl (5-methoxy-1-methyl-1H-indazol-4-yl)carbamate (Intermediate 37-d) as a yellow foam (77.0 mg, 79%). LCMS (ESI) m/z 278.1 [M+H]⁺.

### Step 4

The title compound was made from tert-butyl (5-methoxy-1-methyl-1H-indazol-4-yl)carbamate (Intermediate 37-d) following the procedure as described in Example 4, Step 4. The volatiles were removed under reduced pressure to provide 5-methoxy-1-methy-1-H-indazol-4-amine (Intermediate 37-e) as a brown oil (80.6 mg, crude). LCMS (ESI) m/z 173.0 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 5-methoxy-1-methyl-1H-indazol-4-amine (Intermediate 37-e) was used in place of 1H-indazol-6-amine. The crude product was purified by flash chromatography on silica gel column using 0-50% EtOAc/Hexanes as eluent, to provide the title compound as a white solid (68.0 mg, 69%). ¹H NMR (400 MHz, DMSO-d₆) δ ppm 2.68 (d, J=4.3 Hz, 3H), 3.80 (s, 3H), 4.00 (s, 3H), 6.88-6.91 (m, 1H), 7.30 (d, J=9.0 Hz, 1H), 7.42 (d, J=9.0 Hz, 1H), 7.72 (s, 1H), 8.04 (s, 1H), 8.80 (s, 1H); ¹⁹F NMR (376 MHz, DMSO-d₆) δ ppm −60.0 (s, 3F); LCMS (ESI) m/z 353.2 [M+H]⁺.

### Example 38

### Synthesis of N2-(5-fluoro-1-methyl-1H-indazol-4-yl)-N4-methyl-5-(trifluoromethyl) pyrimidine-2,4-diamine

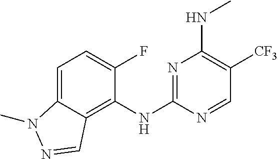 [SMILES: CNc1nc(Nc2c(F)ccc3c2cnn3C)ncc1C(F)(F)F]

The title compound was made from 2-bromo-3,6-difluorobenzaldehyde following the procedure as described in Example 4, Step 1. The crude product was purified by flash chromatography on silica gel column using 0-25% EtOAc/Hexanes as eluent, to provide 4-bromo-5-fluoro-1H-indazole (Intermediate 38-a) as a pale-beige solid (0.21 g, 28%). LCMS (ESI) m/z 216.9 [M+H]⁺.

### Step 2

The title compounds were made from 4-bromo-5-fluoro-1H-indazole (Intermediate 38-a) following the procedure as described in Example 10, Step 1. The crude products (3:2 ratio of separable N1:N2 regioisomers) were purified by flash chromatography on silica gel column using 0-30% EtOAc/Hexanes as eluent, to provide title compounds 4-bromo-5-fluoro-1-methyl-1H-indazole (Intermediate 38-b) (0.15 g, 63%) and 4-bromo-5-fluoro-2-methyl-2H-indazole (Intermediate 38-c) (68.0 mg, 30%) as colorless oils. LCMS (ESI) m/z 231.0 [M+H]⁺.

### Step 3

The title compound was made from 4-bromo-5-fluoro-1-methyl-1H-indazole (Intermediate 38-b) following the procedure as described in Example 4, Step 3. The crude product was purified by flash chromatography on silica gel column using 0-50% EtOAc/Hexanes as eluent, to provide tert-butyl (5-fluoro-1-methyl-1H-indazol-4-yl)carbamate (Intermediate 38-d) as a yellow foam (0.14 g, 88%). LCMS (ESI) m/z 266.1 [M+H].

### Step 4

The title compound was made from tert-butyl (5-fluoro-1-methyl-H-indazol-4-yl)carbamate (Intermediate 38-d) following the procedure as described in Example 4, Step 4. The volatiles were removed under reduced pressure to provide 5-fluoro-1-methyl-1H-indazol-4-amine (Intermediate 38-e) as a brown oil (0.15 g, crude). LCMS (ESI) m/z 166.0 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 5-fluoro-1-methyl-1H-indazol-4-amine (Intermediate 38-e) was used in place of 1H-indazol-6-amine. The crude product was purified by flash chromatography on silica gel column using 0-50% EtOAc/Hexanes as eluent, to provide the title compound as an off-white solid (0.11 g, 62%). ¹H NMR (400 MHz, DMSO-d₆) δ ppm 2.76 (d, J=4.3 Hz, 3H), 4.09 (s, 3H), 7.03-7.06 (m, 1H), 7.39 (dd, J=10.6, 9.0 Hz, 1H), 7.53 (dd, J=9.0, 3.5 Hz, 1H), 7.97 (s, 1H), 8.15 (s, 1H), 9.53 (s, 1H); ¹⁹F NMR (376 MHz, DMSO-d₆) δ ppm −60.3 (s, 3F), −130.3 (s, 1F); LCMS (ESI) m/z 341.1 [M+H]⁺.

### Example 39

### Synthesis of N2-(2,5-dimethyl-2H-indazol-4-yl)-N4-methyl-5-(trifluoromethyl)pyrimidine-2,4-diamine

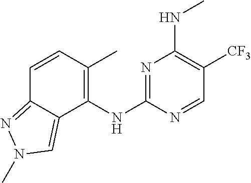 [SMILES: CNc1nc(Nc2c(C)ccc3nn(C)cc23)ncc1C(F)(F)F]

### Step 1

The title compound was made from 4-bromo-2,5-dimethyl-2H-indazole (Intermediate 36-b) following the procedure as described in Example 4, Step 3. The crude product was purified by flash chromatography on silica gel column using 0-80% EtOAc/Hexanes as eluent, to provide tert-butyl (2,5-dimethyl-2H-indazol-4-yl)carbamate (Intermediate 39-a) as an off-white solid (0.30 g, 88%). LCMS (ESI) m/z 262.2 [M+H]⁺.

### Step 2

The title compound was made from tert-butyl (2,5-dimethyl-2H-indazol-4-yl)carbamate (Intermediate 39-a) following the procedure as described in Example 4, Step 4. The volatiles were removed under reduced pressure to provide 2,5-dimethyl-2H-indazol-4-amine (Intermediate 39-b) as a brown oil (0.32 g, crude). LCMS (ESI) m/z 162.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 2,5-dimethyl-2H-indazol-4-amine (Intermediate 39-b) was used in place of 1H-indazol-6-amine. The crude product was purified by flash chromatography on silica gel column using 0-80% EtOAc/Hexanes as eluent, to provide the title compound as a white solid (74.0 mg, 20%) ¹H NMR (400 MHz, DMSO-d₆) δ ppm 2.21 (s, 3H), 2.69 (br s, 3H), 4.09 (s, 3H), 6.85-6.88 (m, 1H), 7.09 (d, J=8.7 Hz, 1H), 7.35 (d, J=8.7 Hz, 1H), 7.99 (s, 1H), 8.03 (s, 1H), 9.11 (s, 1H); ¹⁹F NMR (376 MHz, DMSO-d₆) δ ppm −60.0 (s, 3F); LCMS (ESI) m/z 337.1 [M+H]⁺.

### Example 40

### Synthesis of N2-(5-methoxy-2-methyl-2H-indazol-4-yl)-N-methyl-5-(trifluoromethyl) pyrimidine-2,4-diamine

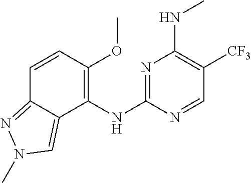 [SMILES: CNc1nc(Nc2c(OC)ccc3nn(C)cc23)ncc1C(F)(F)F]

### Step 1

The title compound was made from 4-bromo-5-methoxy-2-methyl-2H-indazole (Intermediate 37-c) following the procedure as described in Example 4, Step 3. The crude product was purified by flash chromatography on silica gel column using 0-80% EtOAc/Hexanes as eluent, to provide tert-butyl (5-methoxy-2-methyl-2H-indazol-4-yl)carbamate (Intermediate 40-a) as yellow foam (42.6 mg, 99%). LCMS (ESI) m/z 278.1 [M+H]⁺.

### Step 2

The title compound was made from tert-butyl (5-methoxy-2-methyl-2H-indazol-4-yl)carbamate (Intermediate 40-a) following the procedure as described in Example 4, Step 4. The volatiles were removed under reduced pressure to provide 5-methoxy-2-methyl-2H-indazol-4-amine (Intermediate 40-b) as a brown oil (44.6 mg, crude). LCMS (ESI) m/z 178.1 [M+H]⁺.

Step 3

The title compound was made from 2-chloro-N-methyl-5-(trifluoromethyl)pyrimidin-4-amine (Intermediate 1-a) following the procedure as described in Example 1, Step 2, except that 5-methoxy-2-methyl-2H-indazol-4-amine (Intermediate 40-b) was used in place of 1H-indazol-6-amine. The crude product was purified by flash chromatography on silica gel column using 0-80% EtOAc/Hexanes as eluent, to provide the title compound as a pale-yellow solid (14.0 mg, 26%). ¹H NMR (400 MHz, DMSO-d₆) δ ppm 2.69 (d, J=4.3 Hz, 3H), 3.78 (s, 3H), 4.09 (s, 3H), 6.86-6.89 (m, 1H), 7.21 (d, J=9.2 Hz, 1H), 7.43 (d, J=9.2 Hz, 1H), 8.02 (s, 1H), 8.03 (s, 1H), 8.70 (s, 1H); ¹⁹F NMR (376 MHz, DMSO-d₆) δ ppm −60.0 (s, 3F); LCMS (ESI) m/z 353.1 [M+H]⁺.

### Example 41

### Synthesis of N2-(5-fluoro-2-methyl-2H-indazol-4-yl)-N4-methyl-5-(trifluoromethyl) pyrimidine-2,4-diamine

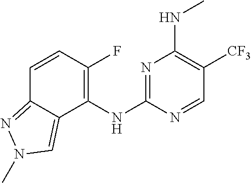 [SMILES: CNc1nc(Nc2c(F)ccc3nn(C)cc23)ncc1C(F)(F)F]

### Step 1

The title compound was made from 4-bromo-5-fluoro-2-methyl-2H-indazole (Intermediate 38-c) following the procedure as described in Example 4, Step 3. The crude product was purified by flash chromatography on silica gel column using 0-80% EtOAc/Hexanes as eluent, to provide tert-butyl (5-fluoro-2-methyl-2H-indazol-4-yl)carbamate (Intermediate 41-a) as yellow foam (50.0 mg, 63%). LCMS (ESI) m/z 266.2 [M+H]⁺.

### Step 2

The title compound was made from tert-butyl (5-fluoro-2-methyl-2H-indazol-4-yl)carbamate (Intermediate 41-a) following the procedure as described in Example 4, Step 4. The volatiles were removed under reduced pressure to provide 5-fluoro-2-methyl-2H-indazol-4-amine (Intermediate 41-b) as a brown oil (52.4 mg, crude). LCMS (ESI) m/z 166.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 5-fluoro-2-methyl-2H-indazol-4-amine (Intermediate 41-b) was used in place of 1H-indazol-6-amine. The crude product was purified by flash chromatography on silica gel column using 0-80% EtOAc/Hexanes as eluent, to provide the title compound as a white solid (22.0 mg, 34%). ¹H NMR (400 MHz, DMSO-d₆) δ ppm 2.70 (d, J=4.2 Hz, 3H), 4.13 (s, 3H), 6.95-6.98 (m, 1H), 7.17 (dd, J=10.8, 9.2 Hz, 1H), 7.45 (dd, J=9.2, 3.9 Hz, 1H), 8.08 (s, 1H), 8.21 (s, 1H), 9.32 (s, 1H); ¹⁹F NMR (376 MHz, DMSO-d₆) δ ppm −60.2 (s, 3F), −129.1 (s, 1F); LCMS (ESI) m/z 341.1 [M+H]⁺.

### Example 42

### Synthesis of N2-(6-fluoro-1-methyl-1H-indazol-4-yl)-N4-methyl-5-(trifluoromethyl) pyrimidine-2,4-diamine

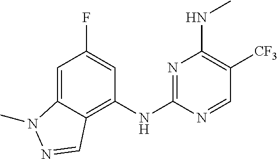 [SMILES: CNc1nc(Nc2cc(F)cc3c2cnn3C)ncc1C(F)(F)F]

### Step 1

The title compounds were made from 6-fluoro-4-nitro-1H-indazole following the procedure as described in Example 10, Step 1. The crude products (3:2 ratio of separable N1:N2 regioisomers) were purified by flash chromatography on silica gel column using 0-60% EtOAc/Hexanes as eluent, to provide 6-fluoro-1-methyl-4-nitro-1H-indazole (Intermediate 42-a) (0.23 g, 55%) and 6-fluoro-2-methyl-4-nitro-2H-indazole (Intermediate 42-b) (0.15 g, 36%) as yellow solids. LCMS (ESI) m/z 196.1 [M+H]⁺.

### Step 2

The title compound was made from 6-fluoro-1-methyl-4-nitro-1H-indazole (Intermediate 42-a) following the procedure as described in Example 7, Step 1. The volatiles were removed under reduced pressure to provide 6-fluoro-1-methyl-1-indazol-4-amine (Intermediate 42-c) as a brown oil (0.19 g, crude). LCMS (ESI) m/z 166.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 6-fluoro-1-methyl-1H-indazol-4-amine (Intermediate 42-c) was used in place of 1H-indazol-6-amine. The crude product was triturated with MeCN (×2), to provide the title compound as an off-white powder (0.22 g, 58%). ¹H NMR (400 MHz, DMSO-d₆) δ ppm 298 (d, J=4.4 Hz, 3H), 3.96 (s, 3H), 7.09 (ddd, J=9.1, 2.1, 1.0 Hz, 1H), 7.30-7.33 (m, 1H), 8.04 (dd, J=13.3, 2.1 Hz, 1H), 8.28 (d, J=1.0 Hz, 1H), 8.52 (d, J=0.9 Hz, 1H), 10.04 (s, 1H); ¹⁹F NMR (376 MHz, DMSO-d₆) δ ppm −60.7 (s, 3F), −113.2 (s, 1F); LCMS (ESI) m/z 341.1 [M+H]⁺.

### Example 4

### Synthesis of N2-(6-isopropyl-1-methyl-1H-indazol-4-yl)-N4-methyl-5-(trifluoromethyl) pyrimidine-2,4-diamine

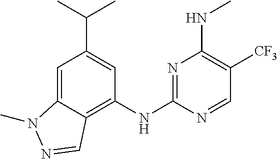 [SMILES: CNc1nc(Nc2cc(C(C)C)cc3c2cnn3C)ncc1C(F)(F)F]

### Step 1

The title compounds were made from 6-bromo-4-nitro-1H-indazole following the procedure as described in Example 10, Step 1. The crude products (3:2 ratio of separable N1:N2 regioisomers) were purified by flash chromatography on silica gel column using 0-30% EtOAc/Hexanes as eluent, to provide 6-bromo-1-methyl-4-nitro-1H-indazole (Intermediate 43-a) (0.68 g, 64%) and 6-bromo-2-methyl-4-nitro-2H-indazole (Intermediate 43-b) (0.36 g, 34%) as yellow solids. LCMS (ESI) m/z 258.1 [M+H]⁺.

### Step 2

To a stirring solution of 6-bromo-1-methyl-4-nitro-1H-indazole (Intermediate 43-a) (100 mg, 391 μmol, 1.00 eq) in a mixture of DMF (1.30 mL) and H₂O (255 μL), was added isopropenylboronic acid pinacol ester (90.8 μl, 469 μmol, 1.20 eq), Cs₂CO₃ (382 mg, 1.17 mmol, 3.00 eq), and Pd(PPh₃)₄ (45.6 mg, 39.1 μmol, 0.10 eq). The reaction mixture was degassed under reduced pressure with backflow of argon (3×), warmed to 85° C., and stirred for 1 h. The reaction mixture was cooled to room temperature, filtered over Celite, and concentrated under reduced pressure to provide 1-methyl-4-nitro-6-(prop-1-en-2-yl)-1H-indazole (Intermediate 43-c) as a colorless oil (84.8 mg, crude). LCMS (ESI) m/z 218.1 [M+H]⁺.

### Step 3

To a stirring solution of 1-methyl-4-nitro-6-(prop-1-en-2-yl)-1H-indazole (Intermediate 43-c) (84.0 mg, 387 μmol, 1.00 eq) in EtOH (3.90 mL), was added palladium (10 wt. %) on carbon (41.2 mg, 38.7 μmol, 0.10 eq). The reaction mixture was degassed under reduced pressure with backflow of hydrogen (3×) and stirred for 7 h under standard hydrogen atmosphere. The reaction mixture was filtered over Celite and concentrated under reduced pressure to provide 6-isopropyl-1-methyl-1H-indazol-4-amine (Intermediate 43-d) as a colorless oil (73.2 mg, crude). LCMS (ESI) m/z 190.1 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 6-isopropyl-1-methyl-1H-indazol-4-amine (Intermediate 43-d) was used in place of 1H-indazol-6-amine. The crude product was purified by flash chromatography on silica gel column using 0-50% EtOAc/Hexanes as eluent, to provide the title compound as an off-white solid (27.5 mg, 20%). ¹H NMR (400 MHz, DMSO-d₆) δ ppm 1.28 (d, J=6.9 Hz, 6H), 2.96-3.01 (m, 4H), 3.97 (s, 3H), 7.07 (s, 1H), 7.20 (m, 1H), 7.93 (s, 1H), 8.22 (s, 1H), 8.31 (d, J=0.9 Hz, 1H), 9.72 (s, 1H); ¹⁹F NMR (376 MHz, DMSO-d₆) δ ppm −60.4 (s, 3F); LCMS (ESI) m/z 365.1 [M+H]⁺.

### Example 44

### Synthesis of 1-methyl-4-((4-(methylamino)-5-(trifluoromethyl)pyrimidin-2-yl)amino)-1H-indazole-6-carbonitrile

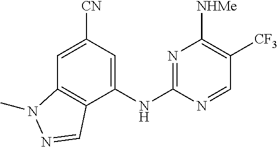 [SMILES: CNc1nc(Nc2cc(C#N)cc3c2cnn3C)ncc1C(F)(F)F]

### Step 1

To a solution of 6-bromo-1-methyl-4-nitro-1H-indazole (Intermediate 43-a) (0.12 g, 0.47 mmol, 1.00 eq) in DMA (5.0 mL) sparged with argon, was added Zn(CN)₂ (55.0 mg, 0.47 mmol, 1.00 eq) and Pd(PPh₃)₄ (54.2 mg, 0.05, 0.11 eq). The reaction mixture was flushed with argon, warmed to 140° C., and stirred for 1 h. After cooling to room temperature, the reaction mixture was poured into water (15 mL) and extracted with EtOAc (15 ml×3). The combined organic layers were washed with brine (20 mL×3), dried over anhydrous MgSO₄, filtered, and concentrated under reduced pressure. The crude product was purified by flash chromatography on silica gel column using 0-40% EtOAc/Hexanes as eluent, to provide 1-methyl-4-nitro-1H-indazole-6-carbonitrile (Intermediate 44-a) as a white solid (85.0 mg, 90%). ¹H NMR (400 MHz, DMSO-dt) δ ppm 4.24 (s, 3H), 8.53 (d, J=1.1 Hz, 1H), 8.63 (d, J=1.0 Hz, 1H), 8.99 (t, J=1.0 Hz, 1H).

### Step 2

The title compound was made from 1-methyl-4-nitro-1H-indazole-6-carbonitrile (Intermediate 44-a) following the procedure as described in Example 7, Step 1. The crude product was purified by flash chromatography on silica gel column using 0-50% EtOAc/Hexanes as eluent, to provide 4-amino-1-methyl-1H-indazole-6-carbonitrile (Intermediate 44-b) as an orange oil (20.0 mg, 29%). LCMS (ESI) m/z 173.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 4-amino-1-methyl-1H-indazole-6-carbonitrile (Intermediate 44-b) was used in place of 1H-indazol-6-amine. The crude product was purified by reverse phase preparative HPLC (Gemini® 5 um NX-C18 110 Å, 100×30 mm; mobile phase: [water (10 mM Ammonium Formate pH=3.8 v/v)-MeCN]; B %: 35%-100%, 16 min), to provide the title compound as a white solid (10.0 mg, 25%). ¹H NMR (400 MHz, DMSO-d₆) δ ppm 2.99 (d, J=4.3 Hz, 3H), 4.08 (s, 3H), 7.36 (s, 1H), 7.94 (s, 1H), 8.30 (s, 1H), 8.40 (s, 1H), 8.65 (s, 1H), 10.17 (s, 1H); ¹⁹F NMR (376 MHz, DMSO-d₆) δ ppm −60.8 (s, 3F); LCMS (ESI) m/z 348.2 [M+H]⁺.

### Example 45

### Synthesis of N2-(1,6-dimethyl-1H-indazol-4-yl)-N4-methyl-5-(trifluoromethyl)pyrimidine-2,4-diamine

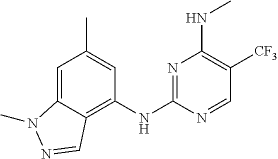 [SMILES: CNc1nc(Nc2cc(C)cc3c2cnn3C)ncc1C(F)(F)F]

### Step 1

The title compound was made from 6-bromo-1-methyl-4-nitro-1H-indazole (Intermediate 43-a) following the procedure as described in Example 43, Step 2, except that trimethylboroxine was used in place of isopropenylboronic acid pinacol ester. The crude product was purified by flash chromatography on silica gel column using 0-50% EtOAc/Hexanes as eluent, to provide 1,6-dimethyl-4-nitro-1H-indazole (Intermediate 45-a) as yellow solid (46.0 mg, 62%). LCMS (ESI) m/z 192.1 [M+H]⁺.

### Step 2

The title compound was made from 1,6-dimethyl-4-nitro-1H-indazole (Intermediate 45-a) following the procedure as described in Example 7, Step 1. The volatiles were removed under reduced pressure to provide 1,6-dimethyl-1H-indazol-4-amine (intermediate 45-b) as a brown oil (33.7 mg, crude). LCMS (ESI) m/z 162.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 1,6-dimethyl-1H-indazol-4-amine (Intermediate 45-b) was used in place of 1H-indazol-6-amine. The crude product was purified by flash chromatography on silica gel column using 0-50% EtOAc/Hexanes as eluent, to provide the title compound as a white solid (253 mg, 49%). 1H NMR (400 MHz, DMSO-d₆) δ ppm 2.42 (s, 3H), 2.95 (d, J=4.4 Hz, 3H), 3.95 (s, 3H), 7.01-7.02 (m, 1H), 7.17-7.20 (m, 1H), 7.86 (s, 1H), 8.22 (d, J=1.0 Hz, 1H), 8.30 (d, J=0.9 Hz, 1H), 9.72 (s, 1H); ¹⁹F NMR (376 MHz, DMSO-d₆) δ ppm −60.4 (s, 3F); LCMS (ESI) m/z 337.1 [M+H]⁺.

### Synthesis of (E)-3-(1-methyl-4-((4-(methylamino)-5-(trifluoromethyl)pyrimidin-2-yl)amino)-1H-indazol-6-yl)-1-morpholinoprop-2-en-1-one

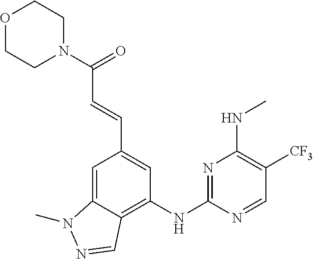 [SMILES: CNc1nc(Nc2cc(/C=C/C(=O)N3CCOCC3)cc3c2cnn3C)ncc1C(F)(F)F]

### Step 1

To a stirring solution of 6-bromo-1-methyl-4-nitro-1H-indazole (Intermediate 43-a) (100 mg, 391 μmol, 1.00 eq) in DMF (1.00 mL), was added 4-acryloylmorpholine (76.8 μL, 586 μmol, 1.50 eq), triethylamine (219 μL, 1.56 mmol, 4.00 eq), tris(o-tolyl)phosphine (30.3 mg, 97.6 μmol, 0.25 eq), and Pd(OAc)₂ (13.2 mg, 58.6 μmol, 0.15 eq). The reaction mixture was degassed under reduced pressure with backflow of argon (3×), warmed to 100° C., and stirred for 2 h. The reaction mixture was cooled to room temperature, filtered over Celite, and concentrated under reduced pressure. The crude product was purified by flash chromatography on silica gel column using 0-5% MeOH/CH₂Cl₂ as eluent, to provide (E)-3-(1-methyl-4-nitro-1H-indazol-6-yl)-1-morpholinoprop-2-en-1-one (Intermediate 46-a) as a yellow solid (0.12 g, 99%). LCMS (ESI) m/z 317.1 [M+H]⁺.

### Step 2

The title compound was made from (E)-3-(1-methyl-4-nitro-1H-indazol-6-yl)-1-morpholinoprop-2-en-1-one (Intermediate 46-a) following the procedure as described in Example 7, Step 1. The volatiles were removed under reduced pressure to provide (E)-3-(4-amino-1-methyl-1H-indazol-6-yl)-1-morpholinoprop-2-en-1-one (Intermediate 46-b) as a brown oil (0.13 g, crude). LCMS (ESI) m/z 287.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that (E)-3-(4-amino-1-methyl-1H-indazol-6-yl)-1-morpholinoprop-2-en-1-one (Intermediate 46-b) was used in place of 1H-indazol-6-amine. The crude product was triturated with MeCN and the solid purified by flash chromatography on silica gel column using 0-50% EtOAc/Hexanes as eluent, to provide the title compound as a white solid (0.14 g, 67%). ¹H NMR (400 MHz, DMSO-d₆) δ ppm 2.96 (d, J=4.4 Hz, 3H), 3.62 (s, 8H), 4.04 (s, 3H), 7.21-7.25 (m, 2H), 7.62 (d, J=15.3 Hz, 1H), 7.66 (s, 1H), 8.23-8.24 (m, 2H), 8.40 (d, J=0.9 Hz, 1H), 9.84 (s, 1H); ¹⁹F NMR (376 MHz, DMSO-d₆) δ ppm −60.5 (s, 3F); LCMS (ESI) m/z 462.2 [M+H]⁺.

### Example 47

### Synthesis of N2-(7-methoxy-1-methyl-1H-indazol-4-yl)-N4-methyl-5-(trifluoromethyl) pyrimidine-2,4-diamine

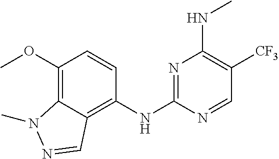 [SMILES: CNc1nc(Nc2ccc(OC)c3c2cnn3C)ncc1C(F)(F)F]

### Step 1

The title compound was made from 4-bromo-7-methoxy-1-methyl-1H-indazole (Intermediate 35-b) following the procedure as described in Example 4, Step 3. The crude product was purified by flash chromatography on silica gel column using 0-40% EtOAc/Hexanes as eluent, to provide tert-butyl (7-methoxy-1-methyl-1H-indazol-4-yl)carbamate (Intermediate 47-a) as a yellow foam (0.26 g, 99%). LCMS (ESI) m/z 278.1 [M+H]⁺.

### Step 2

The title compound was made from tert-butyl (7-methoxy-1-methyl-1H-indazol-4-yl)carbamate (Intermediate 47-a) following the procedure as described in Example 4, Step 4. The volatiles were removed under reduced pressure to provide 7-methoxy-1-methyl-1H-indazol-4-amine (Intermediate 47-b) as a brown oil (0.27 g, crude). LCMS (ESI) m/z 178.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 7-methoxy-1-methyl-1H-indazol-4-amine (Intermediate 47-b) was used in place of 1H-indazol-6-amine. The crude product was purified by reverse phase chromatography on a C18 column using 0-30% MeCN/H₂O (0.1% FA) as eluent, to provide the title compound as a white solid (0.15 g, 44%). ¹H NMR (400 MHz, DMSO-d₆) δ ppm 2.88 (d, J=4.4 Hz, 3H), 3.91 (s, 3H), 4.19 (s, 3H), 6.78 (d, J=8.3 Hz, 1H), 7.05-7.08 (m, 1H), 7.61 (d, J=8.2 Hz, 1H), 8.15 (s, 1H), 8.21 (s, 1H), 9.50 (s, 1H); ¹⁹F NMR (376 MHz, DMSO-d₆) δ ppm −60.3 (s, 3F); LCMS (ESI) m/z 353.1 [M+H]⁺.

### Example 48

### Synthesis of N4-methyl-N2-(3-methyl-1H-indazol-4-yl)-5-(trifluoromethyl)pyrimidine-2,4-diamine

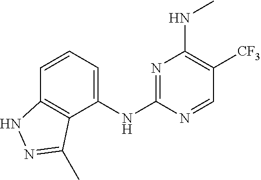 [SMILES: CNc1nc(Nc2cccc3[nH]nc(C)c23)ncc1C(F)(F)F]

### Step 1

The title compound was made from 4-Bromo-3-methyl-1H-indazole following the procedure as described in Example 4, Step 2 to provide 4-bromo-3-methyl-1-(tetrahydro-2H-pyran-2-yl)-1H-indazole (Intermediate 48-a) as a white solid (100.0 mg, 38%). LCMS (ESI) m/z 297.1 [M+H]⁺.

### Step 2

The title compound was made from 4-bromo-3-methyl-1-(tetrahydro-2H-pyran-2-yl)-1H-indazole (intermediate 48-a) following the procedure as described in Example 4, Step 3 to provide tert-butyl (3-methyl-1-(tetrahydro-2H-pyran-2-yl)-1H-indazol-4-yl)carbamate (Intermediate 48-b) as a white solid (102.0 mg, 91%). LCMS (ESI) m/z 332.1 [M+H]⁺.

### Step 3

The title compound was made from tert-butyl (3-methyl-1-(tetrahydro-2H-pyran-2-yl)-1H-imidazol-4-yl)carbamate (Intermediate 48-b) following the procedure as described in Example 4, Step 4 to provide 3-methyl-1H-indazol-4-amine (Intermediate 48-c) as a white solid (100.0 mg, 22%, purity=24%) LCMS (ESI) m/z 148.1 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 3-methyl-1H-indazol-4-amine (Intermediate 48-c) was used in place of 1H-indazol-6-amine. The crude was purified by reverse phase C18 chromatography (H₂O:MeCN (0.1% FA), 95:5->60:40) to provide title compound as a white powder (3.0 mg, 2%). ¹H NMR (400 MHz, DMSO-d₆) δ ppm 2.45 (s, 3H), 2.76 (d, J=4.3 Hz, 3H), 6.97-7.00 (m, 1H), 7.15 (d, J=6.8 Hz, 1H), 7.19-7.26 (m, 2H), 8.09 (s, 1H), 12.56 (s, 1H), 9.10 (s, 1H); ¹⁹F NMR (376 MHz, DMSO-d₆) δ ppm −60.15 (s, 3F); LCMS (ESI) m/z 323.1 [M+H]⁺.

### Example 49

### Synthesis of 4-((4-(methylamino)-5-(trifluoromethyl)pyrimidin-2-yl)amino)-1H-indazole-6-carbonitrile

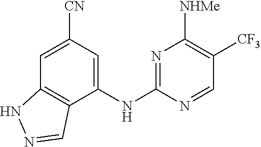 [SMILES: CNc1nc(Nc2cc(C#N)cc3[nH]ncc23)ncc1C(F)(F)F]

### Step 1

The title compound was made from 6-bromo-4-nitro-1H-indazole following the procedure as described in Example 44, Step 1, except that the reaction mixture was warmed to 100° C. and stirred for 24 h. The crude product was purified by flash chromatography on silica gel column using 0-40% EtOAc/Hexanes as eluent, to provide 4-nitro-1H-indazole-6-carbonitrile (Intermediate 49-a) as a brown solid (0.14 g, 58%). LCMS (ESI) m/z 187.0 [M−H]⁻.

### Step 2

The title compound was made from 4-nitro-1H-indazole-6-carbonitrile (Intermediate 49-a) following the procedure as described in Example 7, Step 1. The volatiles were removed under reduced pressure to provide 4-amino-1H-imidazole-6-carbonitrile (Intermediate 49-b) as a pale-yellow solid (0.10 g, crude). LCMS (ESI) m/z 159.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 4-amino-1H-indazole-6-carbonitrile (Intermediate 49-b) was used in place of 1H-indazol-6-amine. The crude product was purified by flash chromatography on silica gel column using 0-100% EtOAc/Hexanes as eluent, to provide the title compound as a pale-yellow solid (26.0 mg, 25%). ¹H NMR (400 MHz, DMSO-d₆) δ ppm 2.98 (d, J=4.4 Hz, 3H), 7.33-7.36 (m, 1H), 7.72 (t, J=1.1 Hz, 1H), 8.30 (d, J=1.0 Hz, 1H), 8.38-8.38 (m, 1H), 8.69 (t, J=1.2 Hz, 1H), 10.17 (s, 1H), 13.53 (s, 1H); ¹⁹F NMR (376 MHz, DMSO-d₆) δ ppm −60.7 (s, 3F); LCMS (ESI) m/z 334.1 [M+H]⁺.

### Example 50

### Synthesis of 2-(4-((4-(methylamino)-5-(trifluoromethyl)pyrimidin-2-yl)amino)-1H-indazol-6-yl)acetonitrile

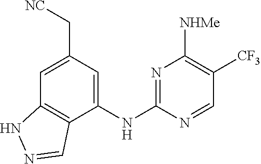 [SMILES: CNc1nc(Nc2cc(CC#N)cc3[nH]ncc23)ncc1C(F)(F)F]

### Step 1

The title compound was made from 6-bromo-4-nitro-1H-indazole following the procedure as described in Example 7, Step 1. The volatiles were removed under reduced pressure to provide 6-bromo-1H-indazol-4-amine (Intermediate 50-a) as an orange oil (0.43 g, crude). LCMS (ESI) m/z 213.9 [M+H]⁺.

### Step 2

To a stirring solution of 6-bromo-1H-indazol-4-amine (Intermediate 50-a) (0.15 g, 0.71 mmol, 1.00 eq) in DMSO (5.0 mL) was added 4-isoxazole boronic acid pinacol ester (0.17 g, 0.87 mmol, 1.23 eq) and an aqueous solution (1 M) of potassium fluoride (2.12 mL, 2.12 mmol, 3.00 eq). After degassing in an ultrasonic bath under argon atmosphere for 5 min, PdCl₂dppf (52.00 mg, 0.07 mmol, 0.10 eq) was added and the reaction mixture was then flushed with argon, warmed to 130° C., and stirred for 16 h. After cooling to room temperature, the reaction mixture was filtered over Celite and washed with 10% MeOH/CH₂Cl₂ (10 mL×2). The filtrate was concentrated under reduced pressure and the residue was then dissolved in EtOAc (20 mL). Water (20 mL) was added, and the mixture was extracted with EtOAc (20 ml×3). The combined organic layers were washed with brine (50 mL), dried over anhydrous MgSO₄, filtered, and concentrated under reduced pressure. The crude product was purified by flash chromatography on silica gel column using 0-60% EtOAc/Hexanes as eluent, to provide 2-(4-amino-1H-indazol-6-yl)acetonitrile (Intermediate 50-b) as a yellow solid (68.0 mg, 56%). LCMS (ESI) m/Z 173.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 2-(4-amino-1H-indazol-6-yl)acetonitrile (Intermediate 50-b) was used in place of 1H-indazol-6-amine. The crude product was purified by reverse phase chromatography on a C18 column using 0-40% MeCN/H₂O (0.1% FA) as eluent, to provide the title compound as an off-white solid (13.0 mg, 10%). ¹H NMR (400 MHz, DMSO-d₆) δ ppm 2.99 (d, J=4.4 Hz, 3H), 4.12 (s, 2H), 7.14 (s, 1H), 7.24 (d, J=5.0 Hz, 1H), 8.04 (s, 1H), 8.24 (s, 1H), 8.47 (s, 1H), 9.89 (s, 1H), 13.02 (br s, 1H); ¹⁹F NMR (376 MHz, DMSO-d₆) δ ppm −60.5 (s, 3F); LCMS (ESI) m/z 348.1 [M+H]⁺.

### Example 51

### Synthesis of 2-(1-methyl-4-((4-(methylamino)-5-(trifluoromethyl)pyrimidin-2-yl)amino)-1H-indazol-6-yl)acetonitrile

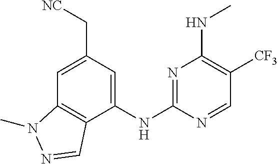 [SMILES: CNc1nc(Nc2cc(CC#N)cc3c2cnn3C)ncc1C(F)(F)F]

### Step 1

The title compound was made from 6-bromo-1-methyl-4-nitro-1H-indazole (Intermediate 43-a) following the procedure as described in Example 7, Step 1. The volatiles were removed under reduced pressure to provide 6-bromo-1-methyl-1H-indazol-4-amine (Intermediate 51-a) as a yellow solid (0.13 g, crude). LCMS (ESI) m/z 228.0 [M+H]⁺.

### Step 2

The title compound was made from 6-bromo-1-methyl-1H-indazol-4-amine (Intermediate 51-a) following the procedure as described in Example 50, Step 2. The crude product was purified by flash chromatography on silica gel column using 0-60% EtOAc/Hexanes as eluent, to provide 2-(4-amino-1-methyl-1H-indazol-6-yl)acetonitrile (Intermediate 51-b) as a yellow solid (65.0 mg, 93%). LCMS (ESI) m/z 187.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 2-(4-amino-1-methyl-1H-indazol-6-yl)acetonitrile (Intermediate 51-b) was used in place of 1H-indazol-6-amine. Trituration of the crude product with acetonitrile provided the title compound as a white solid (27.0 mg, 23%). ¹H NMR (400 MHz, DMSO-d₆) δ ppm 2.99 (d, J=4.4 Hz, 3H), 4.00 (s, 3H), 4.13 (s, 2H), 7.20 (s, 1H), 7.25 (d, J=5.0 Hz, 1H), 8.09 (s, 1H), 8.24 (s, 1H), 8.44 (d, J=0.9 Hz, 1H), 9.92 (s, 1H); ¹⁹F NMR (376 MHz, DMSO-d₆) δ ppm −60.6 (s, 3F); LCMS (ESI) m/z 362.2 [M+H]⁺.

### Example 52

### Synthesis of (E)-3-(4-(4-(methylamino)-5-(trifluoromethyl)pyrimidin-2-yl)amino)-1H-indazol-6-yl)-1-morpholinoprop-2-en-1-one

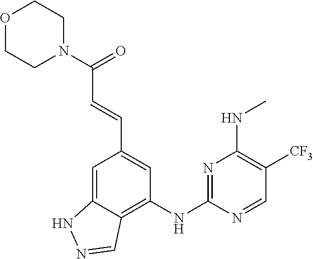 [SMILES: CNc1nc(Nc2cc(/C=C/C(=O)N3CCOCC3)cc3[nH]ncc23)ncc1C(F)(F)F]

### Step 1

The title compound was made from 6-bromo-4-nitro-1H-indazole following the procedure as described in Example 4, Step 2, except that the reaction mixture was warmed to reflux and stirred for 2 h. The crude product was purified by flash chromatography on silica gel column using 0-40% EtOAc/Hexanes as eluent, to provide 6-bromo-4-nitro-1-(tetrahydro-2H-pyran-2-yl)-1H-indazole (Intermediate 52-a) as an orange solid (0.62 g, 92%). ¹HNMR (400 MHz, CHCl₃-d) δ ppm 1.82-1.69 (m, 3H), 2.09-2.05 (m, 1H), 2.30-2.14 (m, 2H), 3.85-3.79 (m, 1H), 4.17-4.14 (m, 1H), 5.73 (dd, J=9.2, 3.0 Hz, 1H), 8.26 (s, 2H), 8.78 (s, 1H).

### Step 2

The title compound was made from 6-bromo-4-nitro-1-(tetrahydro-2H-pyran-2-yl)-1H-indazole (Intermediate 52-a) following the procedure as described in Example 46, Step 1. The crude product was purified by flash chromatography on silica gel column using 0-5% MeOH/CH₂Cl₂ as eluent, to provide (E)-1-morpholino-3-(4-nitro-1-(tetrahydro-2H-pyran-2-yl)-1H-indazol-6-yl)prop-2-en-1-one (Intermediate 52-b) as a yellow solid (0.24 g, 99%). LC MS (ESI) m/z 387.3 [M+H]⁺.

### Step 3

The title compound was made from (E)-1-morpholino-3-(4-nitro-1-(tetrahydro-2H-pyran-2-yl)-1H-indazol-6-yl)prop-2-en-1-one (Intermediate 52-b) following the procedure as described in Example 7, Step 1. The volatiles were removed under reduced pressure to provide (E)-3-(4-amino-1-(tetrahydro-2H-pyran-2-yl)-1H-indazol-6-yl)-1-morpholinoprop-2-en-1-one (Intermediate 52-c) as a brown oil (0.22 g, crude). LCMS (ESI) m/z 357.2 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that (E)-3-(4-amino-1-(tetrahydro-2H-pyran-2-yl)-1H-indazol-6-yl)-1-morpholinoprop-2-en-1-one (Intermediate 52-c) was used in place of 1H-indazol-6-amine. The crude product was purified by reverse phase chromatography on a C18 column using 0-40% MeCN/H₂O (0.1% FA) as eluent, to provide the title compound as a beige solid (45.0 mug, 15%). ¹H NMR (400 MHz, DMSO-d₆) δ ppm 2.96 (d, J=4.4 Hz, 3H), 3.61-3.69 (m, 8H), 7.17 (d, J=15.3 Hz, 1H), 7.21 (m, 1H), 7.47 (s, 1H), 7.62 (d, J=15.3 Hz, 1H), 8.23 (s, 1H), 8.24 (d, J=1.0 Hz, 1H), 8.43 (s, 1H), 9.82 (s, 1H), 13.14 (s, 1H); ¹⁹F NMR (376 MHz, DMSO-d₆) δ ppm −60.4 (s, 3F); LCMS (ESI) m/z 448.3 [M+H]⁺.

### Example 53

### Synthesis of 3-(4-((4-(methylamino)-5-(trifluoromethyl)pyrimidin-2-yl)amino)-1H-indazol-6-yl)-1-morpholinopropan-1-one

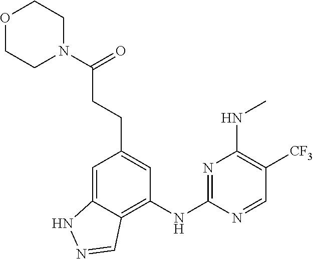 [SMILES: CNc1nc(Nc2cc(CCC(=O)N3CCOCC3)cc3[nH]ncc23)ncc1C(F)(F)F]

To a stirring solution of (E)-3-(4-((4-(methylamino)-5-(trifluoromethyl)pyrimidin-2-yl)amino)-1H-indazol-6-yl)-1-morpholinoprop-2-en-1-one (Example 52) (24.0 mg, 53.6 μmol, 1.00 eq) in a mixture of EtOH (1.40 mL) and NMP (0.72 mL), was added palladium (10 wt. %) on carbon (17.1 mg, 16.1 μmol, 0.30 eq). The reaction mixture was degassed under reduced pressure with backflow of hydrogen (3×) and stirred for 18 h under standard hydrogen atmosphere. The reaction mixture was filtered over Celite and concentrated under reduced pressure. The crude product was purified by reverse phase chromatography on a C18 column using 5-25% MeCN/H₂O (0.1% FA) as eluent, to provide the title compound as a white powder (21.5 mg, 89%). ¹H NMR (400 MHz, DMSO-d₆) δ ppm 2.66 (t, J=7.7 Hz, 2H), 2.90 (t, J=7.6 Hz, 2H), 2.94 (d, J=44 Hz, 3H), 3.34-3.52 (m, 8H), 7.00 (s, 1H), 7.15-7.18 (m, 1H), 7.84 (s, 1H), 8.21 (s, 1H), 8.33 (s, 1H), 9.72 (s, 1H), 12.81 (s, 1H); ¹⁹F NMR (376 MHz, DMSO-d₆) δ ppm −60.4 (s, 3F); LCMS (ESI) m/z 450.3 [M+H]⁺.

### Example 54

### Synthesis of 3-(1-methyl-4-((4-(methylamino)-5-(trifluoromethyl)pyrimidin-2-yl)amino)-1H-indazol-6-yl)-1-morpholinopropan-1-one

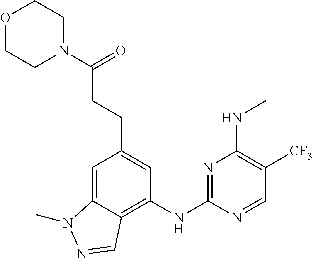 [SMILES: CNc1nc(Nc2cc(CCC(=O)N3CCOCC3)cc3c2cnn3C)ncc1C(F)(F)F]

The title compound was made from (E)-3-(1-methyl-4-((4-(methylamino)-5-(trifluoromethyl)pyrimidin-2-yl)amino)-1H-indazol-6-yl)-1-morpholinoprop-2-en-1-one (Example 46) following the procedure as described in Example 53, Step 1. The crude product was purified by reverse phase chromatography on a C18 column using 5-40% MeCN/H₂O (0.1% FA) as eluent, to provide the title compound as a white powder (82.0 mg, 78%). ¹H NMR (400 MHz, DMSO-d₆) δ ppm 2.68 (t, J=7.9 Hz, 2H), 2.90-2.95 (m, 5H), 3.41-3.49 (m, 8H), 3.96 (s, 3H), 7.10 (s, 1H), 7.17-7.20 (m, 1H), 7.89 (s, 1H), 8.22 (d, J=1.0 Hz, 1H), 8.31 (d, J=0.9 Hz, 1H), 9.74 (s, 1H); ¹⁹F NMR (376 MHz, DMSO-d₆) δ ppm −60.4 (s, 3F); LCMS (ESI) m/z 464.2 [M+H]⁺.

### Example 55

### Synthesis of 2-(1-methyl-4-((4-(methylamino)-5-(trifluoromethyl)pyrimidin-2-yl)amino)-1H-indazol-7-yl)acetonitrile

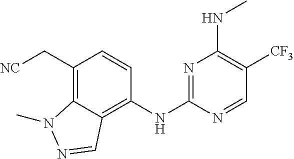 [SMILES: CNc1nc(Nc2ccc(CC#N)c3c2cnn3C)ncc1C(F)(F)F]

### Step 1

The title compound was made from 1-methyl-4-nitro-1H-indazole following the procedure previously reported by Eddahmi et al., New J. Chem., 2019, 43, 14355. The crude product was purified by flash chromatography on silica gel column using 0-10% EtOAc/Hexanes as eluent, to provide 2-(1-methyl-4-nitro-1H-indazol-7-yl)acetonitrile (Intermediate 55-a) as a yellow solid (0.18 g, 38%). ¹H NMR (400 MHz, DMSO-d₆) δ ppm 4.39 (s, 3H), 4.91 (s, 2H), 7.63 (d, J=8.0 Hz, 1H), 8.20 (d, J=7.9 Hz, 1H), 8.52 (s, 1H); LCMS (ESI) m/z 215.1 [M−H]⁻.

### Step 2

The title compound was made from 2-(1-methyl-4-nitro-1H-indazol-7-yl)acetonitrile (Intermediate 55-a) following the procedure as described in Example 7, Step 1. The volatiles were removed under reduced pressure to provide 2-(4-amino-1-methyl-1H-indazol-7-yl)acetonitrile (Intermediate 55-b) as a brown oil (51.0 mg, crude). LCMS (ESI) m/z 187.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 2-(4-amino-1-methyl-1H-indazol-7-yl)acetonitrile (Intermediate 55-b) was used in place of 1H-indazol-6-amine. The crude product was purified by flash chromatography on silica gel column using 0-50% EtOAc/Hexanes as eluent, to provide the title compound as a white solid (52.0 mg, 53%). ¹H NMR (400 MHz, DMSO-d₆) δ ppm 2.93 (d, J=4.4 Hz, 3H), 4.27 (s, 3H), 4.55 (s, 2H), 7.19-7.22 (m, 1H), 7.28 (d, J=8.0 Hz, 1H), 7.92 (d, J=7.9 Hz, 1H), 8.22 (s, 1H), 8.44 (s, 1H), 9.79 (s, 1H); ¹⁹F NMR (376 MHz, DMSO-d₆) δ ppm −60.5 (s, 3F); LCMS (ESI) m/z 362.2 [M+H]⁺.

### Example 56

### Synthesis of (E)-3-(4-((4-(methylamino)-5-(trifluoromethyl)pyrimidin-2-yl)amino)-1H-indazol-7-yl)-1-morpholinoprop-2-en-1-one

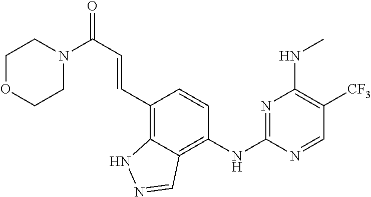 [SMILES: CNc1nc(Nc2ccc(/C=C/C(=O)N3CCOCC3)c3[nH]ncc23)ncc1C(F)(F)F]

### Step 1

To a stirring solution of N2-(1H-indazol-4-yl)-N4-methyl-5-(trifluoromethyl)pyrimidine-2,4-diamine (Example 21) (100 mg, 324 μmol, 1.00 eq) in anhydrous DMF (6.50 mL) was added N-bromosuccinimide (57.7 ng, 324 μmol, 1.00 eq). The reaction mixture was warmed to 40° C. and stirred for 18 h. The reaction mixture was cooled to room temperature and the volatiles removed under reduced pressure. The crude product was triturated with MeCN to provide N2-(7-bromo-1H-indazol-4-yl)-N4-methyl-5-(trifluoromethyl)pyrimidine-2,4-diamine (Intermediate 56-a) as a tan solid (89.0 mg, 71%). ¹H NMR (400 MHz, DMSO-d₆) δ ppm 2.92 (d, J=4.3 Hz, 3H), 7.21 (s, 1H), 7.49 (d, J=8.3 Hz, 1H), 7.87 (d, J=8.2 Hz, 1H), 8.23 (s, 1H), 8.57 (s, 1H), 9.88 (s, 1H), 13.34 (s, 1H); LCMS (ESI) m/z 389.0 [M+H]⁺.

### Step 2

The title compound was made from N2-(7-bromo-1H-indazol-4-yl)-N4-methyl-5-(trifluoromethyl)pyrimidine-2,4-diamine (Intermediate 56-a) following the procedure as described in Example 46, Step 1. The crude product was purified by reverse phase chromatography on a C18 column using 5-40% MeCN/H₂O (01% FA) as eluent, to provide the title compound as a yellow powder (26.0 mg, 28%). ¹H NMR (400 MHz, DMSO-d₆) δ ppm 2.97 (d, J=4.3 Hz, 3H), 3.62-3.77 (m, 8H), 7.20 (d, J=15.4 Hz, 1H), 7.26-7.29 (m, 1H), 7.75 (d, J=8.2 Hz, 1H), 7.92 (d, J=15.3 Hz, 1H), 8.09 (d, J=8.2 Hz, 1H), 8.26 (s, 1H), 8.62 (s, 1H), 10.01 (s, 1H), 13.42 (s, 1H); ¹⁹F NMR (376 MHz, DMSO-d₆) δ ppm −60.3 (s, 3F); LCMS (ESI) m/z 448.3 [M+H]⁺.

### Example 57

### Synthesis of 3-(4-((4-(methylamino)-5-(trifluoromethyl)pyrimidin-2-yl)amino)-1H-indazol-7-yl)-1-morpholinopropan-1-one

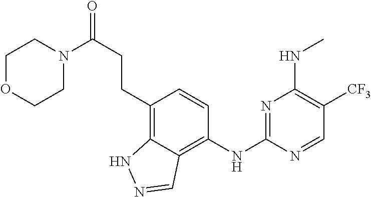 [SMILES: CNc1nc(Nc2ccc(CCC(=O)N3CCOCC3)c3[nH]ncc23)ncc1C(F)(F)F]

The title compound was made from (E)-3-(4-((4-(methylamino)-5-(trifluoromethyl)pyrimidin-2-yl)amino)-1H-indazol-7-yl)-1-morpholinoprop-2-en-1-one (Example 56) following the procedure as described in Example 53, Step 1. The crude product was purified by reverse phase chromatography on a C18 column using 5-25% MeCN/H₂O (0.1% FA) as eluent, to provide the title compound as an off-white powder (9.5 mg, 59%). ¹H NMR (400 MHz, DMSO-d₆) δ ppm 2.70-2.74 (m, 2H), 2.91 (d, J=4.4 Hz, 3H), 3.04-3.08 (m, 2H), 3.38 (m, 2H), 3.43-3.47 (m, 4H), 3.51 (m, 2H), 7.08 (d, J=7.8 Hz, 1H), 7.11-7.14 (m, 1H), 7.74 (d, J=7.7 Hz, 1H), 8.18 (d, J=1.0 Hz, 1H), 8.37 (d, J=1.4 Hz, 1H), 9.65 (s, 1H), 13.02 (s, 1H); ¹⁹F NMR (376 MHz, DMSO-d₆) δ ppm −60.3 (s, 3F); LCMS (ESI) in/z 450.2 [M+H]⁺.

### Example 58

### Synthesis of 3-(1-methyl-4-((4-(methylamino)-5-(trifluoromethyl)pyrimidin-2-yl)amino)-1H-indazol-7-yl)-1-morpholinopropan-1-one

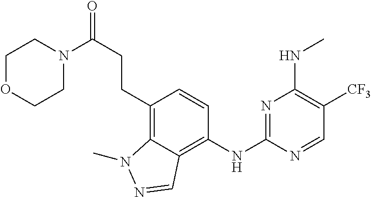 [SMILES: CNc1nc(Nc2ccc(CCC(=O)N3CCOCC3)c3c2cnn3C)ncc1C(F)(F)F]

### Step 1

The title compound was made from N4-methyl-N2-(1-methyl-1H-indazol-4-yl)-5-(trifluoromethyl)pyrimidine-2,4-diamine (Example 21) following the procedure as described in Example 56, Step 1, except that the reaction mixture was warmed to 40° C. and stirred for 30 min. The crude product was triturated with MeCN to provide N2-(7-bromo-1-methyl-1H-indazol-4-yl)-N4-methyl-5-(trifluoromethyl)pyrimidine-2,4-diamine (Intermediate 58-a) as an off-white powder (0.35 g, 90%). LCMS (ESI) m/z 403.0 [M+H]⁺.

### Step 2

The title compound was made from N2-(7-bromo-1-methyl-1H-indazol-4-yl)-N4-methyl-5-(trifluoromethyl)pyrimidine-2,4-diamine (Intermediate 58-a) following the procedure as described in Example 46, Step 1. The crude product was purified by reverse phase chromatography on a C18 column using 5-40% MeCN/H₂O (0.1% FA) as eluent, to provide (E)-3-(1-methyl-4-((4-(methylamino)-5-(trifluoromethyl)pyrimidin-2-yl)amino)-1H-indazol-7-yl)-1-morpholinoprop-2-en-1-one (Intermediate 58-b) as a yellow solid (92.0 mg, 80%). LCMS (ESI) m/z 462.3 [M+H]⁺.

### Step 3

The title compound was made from (E)-3-(1-methyl-4-((4-(methylamino)-5-(trifluoromethyl)pyrimidin-2-yl)amino)-1H-indazol-7-yl)-1-morpholinoprop-2-en-1-one (Intermediate 58-b) following the procedure as described in Example 53, Step 1. The crude product was purified by reverse phase chromatography on a C18 column using 5-30% MeCN/H₂O (0.1% PA) as eluent, to provide the title compound as a white powder (72.0 mg, 80%). ¹H NMR (400 MHz, DMSO-d₆) δ ppm 2.71 (t, J=7.7 Hz, 2H), 2.92 (d, J=4.4 Hz, 3H), 3.26 (t, J=8.1 Hz, 2H), 3.41-3.51 (m, 8H), 4.23 (s, 3H), 7.11 (d, J=7.9 Hz, 1H), 7.13-7.16 (m, 1H), 7.77 (d, J=7.8 Hz, 1H), 8.19 (d, J=1.0 Hz, 1H), 8.34 (s, 1H), 9.62 (s, 1H); ¹⁹F NMR (376 MHz, DMSO-d₆) δ ppm −60.4 (s, 3F); LCMS (ESI) m/z 464.3 [M+H]⁺.

### Example 59

### Synthesis of (E)-3-(1-methyl-4-((4-(methylamino)-5-(trifluoromethyl)pyrimidin-2-yl)amino)-1H-indazol-7-yl)-1-morpholinobut-2-en-1-one

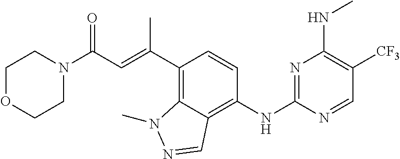 [SMILES: CNc1nc(Nc2ccc(/C(C)=C/C(=O)N3CCOCC3)c3c2cnn3C)ncc1C(F)(F)F]

### Step 1

To a stirring solution of N2-(7-bromo-1-methyl-1H-indazol-4-yl)-N4-methyl-5-(trifluoromethyl)pyrimidine-2,4-diamine (Intermediate 58-a) (300 mg, 748 μmol, 1.00 eq) in DMF (2.50 mL), was added ethyl crotonate (279 μL, 2.24 mmol, 3.00 eq), triethylamine (524 μL, 374 mmol, 5.00 eq), tetrabutylammonium chloride (221 mg, 748 μmol, 1.00 eq), tris(o-tolyl)phosphine (46.4 mg, 150 μmol, 0.20 eq), and Pd(OAc)₂ (17.1 mg, 74.8 μmol, 0.10 eq). The reaction mixture was degassed under reduced pressure with backflow of argon (3×), warmed to 100° C., and stirred for 18 h. The reaction mixture was cooled to room temperature, filtered over Celite, and concentrated under reduced pressure. The crude product was purified by flash chromatography on silica gel column using 0-40% EtOAc/Hexanes as eluent, to provide (E)-3-(1-methyl-4-((4-(methylamino)-5-(trifluoromethyl)pyrimidin-2-yl)amino)-1H-indazol-7-yl)but-2-enoate (Intermediate 59-a) as a yellow powder (0.10 g, 31%). LCMS (ESI) m/z 435.2 [M+H]⁺.

### Step 2

To a stirring suspension of (E)-3-(1-methyl-4-((4-(methylamino)-5-(trifluoromethyl)pyrimidin-2-yl)amino)-1H-indazol-7-yl)but-2-enoate (Intermediate 59-a) (95.0 mg, 219 μmol, 1.00 eq) in a mixture of THF (875 μl), H₂O (875 μl), and MeOH (437 μL), was added LiOH (26.2 mg, 1.09 mmol, 5.00 eq). The reaction mixture was stirred at room temperature for 18 h. The volatiles were removed under reduced pressure and the aqueous layer was acidified with HCl (1N). The resulting precipitate was collected by vacuum filtration to provide (E)-3-(1-methyl-4-((4-(methylamino)-5-(trifluoromethyl)pyrimidin-2-yl)amino)-1H-indazol-7-yl)but-2-enoic acid (Intermediate 59-b as an off-white solid (80.0 mg, 90%) LCMS (OES) m/z 407.2 [M+H]⁺.

### Step 3

To a stirring solution of (F)-3-(1-methyl-4-((4-(methylamino)-5-(trifluoromethyl)pyrimidin-2-yl)amino)-1H-indazol-7-yl)but-2-enoic acid (Intermediate 59-b) (85.0 mg, 209 μmol, 1.00 eq) in anhydrous DMF (4.20 mL), was added morpholine (18.5 μL, 209 μmol, 1.00 eq), 1-methylimidazole (62.6 mg, 732 μmol, 3.50 eq), 1,8-Diazabicyclo[5.4.0]undec-7-ene (31.3 μl, 209 μmol, 1.00 eq), and chloro-N,N,N′,N′-tetramethylformamidinium hexafluorophosphate (65.9 mg, 230 μmol, 1.10 eq). The reaction mixture was stirred for 16 h at room temperature and then partitioned between water (15 mL) and EtOAc (15 mL). The layers were separated, and the aqueous layer was extracted with EtOAc (10 ml×2). The combined organic layers were washed with brine, dried over MgSO₄, filtered, and concentrated under reduced pressure. The crude product was purified by reverse phase chromatography on a C18 column using 5-30% MeCN/H₂O (0.1% FA) as eluent, to provide the title compound as a white powder (65.0 mg, 65%). ¹H NMR (400 MHz, DMSO-d₆) δ ppm 2.24 (d, J=1.3 Hz, 3H), 2.94 (d, J=4.4 Hz, 3H), 353-359 (m, 8H), 3.99 (s, 3H), 6.17-6.18 (m, 1H), 7.13 (d, J=7.9 Hz, 1H), 7.18-7.21 (m, 1H), 7.92 (d, J=7.9 Hz, 1H), 8.22-8.22 (m, 1H), 8.46 (s, 1H), 9.79 (s, 1H) ¹⁹F NMR (376 MHz, DMSO-d₆) δ ppm −60.5 (s, 3F); LCMS (ESI) m/z 476.4 [M+H]⁺.

### Example 60

### Synthesis of 3-(1-methyl-4-((4-(methylamino)-5-(trifluoromethyl)pyrimidin-2-yl)amino)-1H-indazol-7-yl)-1-morpholinobutan-1-one

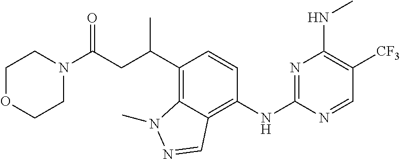 [SMILES: CNc1nc(Nc2ccc(C(C)CC(=O)N3CCOCC3)c3c2cnn3C)ncc1C(F)(F)F]

The title compound was made from (E)-3-(1-methyl-4-((4-(methylamino)-5-(trifluoromethyl)pyrimidin-2-yl)amino)-1H-indazol-7-yl)-1-morpholinobut-2-en-1-one (Example 59) following the procedure as described in Example 53, Step 1, except the reaction mixture was stirred for seven days. The crude product was purified by reverse phase chromatography on a C18 column using 5-30% MeCN/H₂O (0.1% FA) as eluent, followed by reverse phase preparative HPLC (Gemini® 5 um NX-C18 110 Å, 100×30 mm; mobile phase: [water (10 mM Ammonium Bicarbonate pH=10.0 v/v)-MeCN]; B %: 45%-100%, 16 min) to provide the title compound as a white powder (9.0 mg, 18%). ¹H NMR (400 MHz, DMSO-d₆) δ ppm 1.32 (d, J=6.8 Hz, 3H), 2.66 (dd, J=15.9, 7.2 Hz, 1H), 2.86 (dd, J=15.9, 6.7 Hz, 1H), 2.93 (d, J=4.3 Hz, 3H), 3.36-3.49 (m, 8H), 4.06-4.15 (m, 1H), 4.27 (s, 3H), 7.13-7.16 (m, 1H), 7.25 (d, J=8.0 Hz, 1H), 7.82 (d, J=8.0 Hz, 1H), 8.18 (s, 1H), 8.33 (s, 1H), 9.61 (s, 1H); ¹⁹F NMR (376 MHz, DMSO-d₆) δ ppm −60.3 (s, 3F); LCMS (ESI) m/z 478.4 [M+H]⁺.

### Example 61

### Synthesis of N4-methyl-N2-(pyrazolo[1,5-a]pyridin-3-yl)-5-(trifluoromethyl)pyrimidine-2,4-diamine

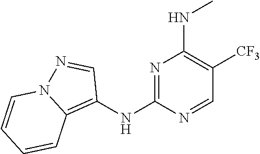 [SMILES: CNc1nc(Nc2cnn3ccccc23)ncc1C(F)(F)F]

The title compound was made from 2-chloro-N-methyl-5-(trifluoromethyl)pyrimidin-4-amine (Intermediate 1-a) following the procedure as described in Example 1, Step 2, except that Pyrazolo[1,5-a]pyridin-3-amine was used in place of 1H-indazol-6-amine. The crude product was purified by reverse phase chromatography on a C18 column using 5-20% MeCN/H₂O (0.1% FA) as eluent, to provide the title compound as an off-white powder (96.0 mg, 43%). ¹H NMR (400 MHz, DMSO-d₆) δ ppm 2.88 (br s, 3H), 6.80 (td, J=6.8, 1.4 Hz, 1H), 7.06 (br s, 1H), 7.09 (ddd, J=9.0, 6.6, 1.1 Hz, 1H), 7.86 (br s, 1H), 8.13 (s, 1H), 8.43 (br s, 1H), 8.53 (d, J=7.0 Hz, 1H), 9.70 (br s, 1H); ¹⁹F NMR (376 MHz, DMSO-d₆) δ ppm −60.0 (s, 3F); LCMS (ESI) m/z 309.0 [M+H]⁺.

### Example 62

### Synthesis of N4-methyl-N2-(2-methylpyrazolo[1,5-a]pyridin-3-yl)-5-(trifluoromethyl) pyrimidine-2,4-diamine

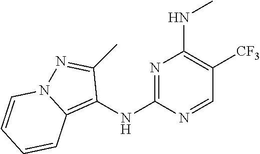 [SMILES: CNc1nc(Nc2c(C)nn3ccccc23)ncc1C(F)(F)F]

### Step 1

To a stirring solution of pyridine (1.02 mL, 12.6 mmol, 1.00 eq) in MeCN (70.1 mL), was added O-(2,4-dinitrophenyl)hydroxylamine (2.89 g, 14.5 mmol, 1.15 eq). The reaction mixture was warmed to 40° C. and stirred for 24 h. The reaction mixture was cooled to room temperature and the volatiles were removed under reduced pressure. The crude residue was dissolved in DMF (70.1 mL) and ethyl 2-butynoate (2.25 mL, 18.9 mmol, 1.50 eq) and potassium carbonate (3.49 g, 25.2 mmol. 2.00 eq) were added. The reaction mixture was stirred at room temperature for 3 h and then partitioned between water (100 mL) and EtOAc (100 mL). The layers were separated, and the aqueous layer was extracted with EtOAc (100 mL×2). The combined organic layers were washed with brine, dried over MgSO₄, filtered, and concentrated under reduced pressure. The crude product was purified by flash chromatography on silica gel column using 0-25% EtOAc/Hexanes as eluent, to provide ethyl 2-methylpyrazolo[1,5-a]pyridine-3-carboxylate (1.37 g, 6.71 mmol, 1.00 eq) (Intermediate 62-a) as a yellow solid (1.39 g, 54%). LCMS (ESI) m/z 205.1 [M+H]⁺.

### Step 2

To a stirring solution of ethyl 2-methylpyrazolo[1,5-a]pyridine-3-carboxylate (1.37 g, 6.71 mmol, 1.00 eq) (Intermediate 62-a) in a mixture of THF (10.1 mL) and MeOH (6.71 mL), was added aqueous NaOH (7.5 M) (2.68 mL, 20.1 mmol, 3.00 eq). The reaction mixture was warmed to 70° C. and stirred for 5 h. The reaction mixture was cooled to room temperature, concentrated under reduced pressure, and acidified with HCl (1N). The resulting precipitate was collected by vacuum filtration to provide 2-methylpyrazolo[1,5-a]pyridine-3-carboxylic acid (Intermediate 62-b) as an off-white solid (1.14 g, 96%). LCMS (ESI) m/z 177.1 [M+H]⁺.

### Step 3

To a stirring solution of 2-methylpyrazolo[1,5-a]pyridine-3-carboxylic acid (Intermediate 62-b) (600 mg, 3.41 mmol, 1.00 eq) in tBuOH (22.7 mL) was added triethylamine (954 uL, 6.81 mmol, 2.00 eq) and diphenylphosphoryl azide (1.36 mL, 6.13 mmol, 1.80 eq). The reaction mixture was stirred at room temperature for 16 h and then warmed to 80° C. and stirred for another 24 h. The reaction mixture was cooled to room temperature, filtered, and the volatiles removed under reduced pressure. The crude product was purified by flash chromatography on silica gel column using 0-50% EtOAc/Hexanes as eluent, to provide tert-butyl (2-methylpyrazolo[1,5-a]pyridin-3-yl)carbamate (Intermediate 62-c) as a pale-yellow solid (0.49 g, 29%). LCMS (ESI) m/z 248.1 [M+H]⁺.

### Step 4

The title compound was made from tert-butyl (2-methylpyrazolo[1,5-a]pyridin-3-yl)carbamate (Intermediate 62-c) following the procedure as described in Example 4, Step 4. The volatiles were removed under reduced pressure to provide 2-methylpyrazolo[1,5-a]pyridin-3-amine (Intermediate 62-d) as a brown oil (83.3 mg, crude). LCMS (ESI) m/z 175.0 [M+H]⁺.

### Step 5

To a stirring solution of 2-chloro-N-methyl-5-(trifluoromethyl)pyrimidin-4-amine (Intermediate 1-a) (58.2 mg, 275 μmol, 1.00 eq) in anhydrous DMA (1.00 mL) sparged with argon, was added 2-methylpyrazolo[1,5-a]pyridin-3-amine (Intermediate 62-d) (44.6 mg, 303 μmol, 1.10 eq), Cs₂CO₃ (269 mg, 826 μmol, 3.00 eq), Pd₂(dba)₃ (25.2 mg, 25.2 μmol, 0.10 eq), and Xantphos (32.5 mg, 55.0 μmol, 0.20 eq). The reaction mixture was degassed under reduced pressure with backflow of argon (3×), warmed to 100° C., and stirred for 2 h. The reaction mixture was cooled to room temperature, filtered over Celite, and concentrated under reduced pressure. The crude product was purified by reverse phase preparative HPLC (Gemini® 5 um NX-C18 110 Å, 100×30 mm; mobile phase: [water (10 mM Ammonium Bicarbonate pH=10.0 v/v)-MeCN], B: 30%-100%, 16 min), to provide the title compound as a white powder (10.0 mg, 11%). ¹H NMR (400 MHz, DMSO-d₆) δ ppm 2.26 (br s, 3H), 2.92 (s, 1H), 3.32 (s, 3H), 6.75 (t, J=6.8 Hz, 1H), 6.91 (m, 1H), 7.09 (t, J=7.8 Hz, 1H), 7.32 (s, 1H), 7.93-8.96 (m, 1H), 8.46-8.48 (m, 1H); ¹⁹F NMR (376 MHz, DMSO-d₆) δ ppm −60.0 (s, 3F); LCMS (ESI) m/z 323.0 [M+H]⁺.

### Example 63

### Synthesis of N4-methyl-N2-(2-methyl-1H-indol-3-yl)-5-(trifluoromethyl)pyrimidine-2,4-diamine

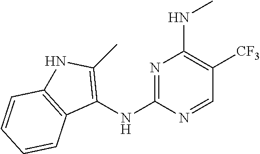 [SMILES: CNc1nc(Nc2c(C)[nH]c3ccccc23)ncc1C(F)(F)F]

### Step 1

The title compound was made from 1-Bromo-2-nitrobenzene following the procedure previously reported by Dobbs, A. J. Org. Chem. 2001, 66 (2) 638. The crude product was purified by flash chromatography on silica gel column using 1-10% EtOAc/Hexanes as eluent, to provide 7-bromo-2-methyl-1H-indole (Intermediate 63-a) as a yellow oil (1.20 g, 58%). LCMS (ESI) m/z 212.0 [M+H]⁺.

### Step 2

To a stirring solution of 7-bromo-2-methyl-1H-indole (Intermediate 63-a) (1.20 g, 5.71 mmol, 1.00 eq) in MeCN (11.4 mL) cooled to 0° C., was added benzoyl chloride (697 μL, 6.00 mmol, 1.05 eq) and silver nitrate (1.02 g, 6.00 mmol, 1.05 eq). The reaction mixture was stirred for 45 min at 0° C. and then partitioned between water (20 ml) and EtOAc (20 mL). The aqueous layer was extracted with EtOAc (20 mL×3) and the combined organic layers were washed with brine, dried over MgSO₄, filtered, and the volatiles removed under reduced pressure. The crude product was triturated with a mixture of MTBE and Heptanes to provide 7-bromo-2-methyl-3-nitro-1H-indole (Intermediate 63-b) as a brown solid (0.79 g, 54%). ¹H NMR (400 MHz, CH₃OH-d₄) δ ppm 2.85 (s, 3H), 7.20 (t, J=7.9 Hz, 1H), 7.45 (d, J=7.9 Hz, 1H), 8.12 (d, 8.0 Hz, 1H).

### Step 3

The title compound was made from 7-bromo-2-methyl-3-nitro-1H-indole (Intermediate 63-b) following the procedure as described in Example 43, Step 3. The volatiles were removed under reduced pressure to provide 2-methyl-1H-indol-3-amine (Intermediate 63-c) as a brown oil (0.12 g, crude). LCMS (ESI) m/z 147.0 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 2-methyl-1H-indol-3-amine (intermediate 63-c) was used in place of 1H-indazol-6-amine. The crude product was purified by flash chromatography on silica gel column using 0-50% EtOAc/Hexanes as eluent, to provide the title compound as an off-white solid (83.5 mg, 53%)¹H NMR (400 MHz, DMSO-d₆) δ (ppm 2.23 (s, 3H), 2.92 (br s, 1H), 3.28 (s, 3H), 6.77 (br s, 1H), 6.88 (d, J=7.7 Hz, 1H), 6.96 (t, J=7.6 Hz, 1H), 7.21 (d, J=8.2 Hz, 2H), 7.87-8.74 (m, 1H), 10.78 (s, 1H); ¹⁹F NMR (376 MHz, DMSO-d.&) δ ppm −59.8 (s, 3F), LCMS (ESI) m/z 322.1 [M+H]⁺.

### Example 64

### Synthesis of N2-(1,2-dimethyl-1H-indol-3-yl)-N4-methyl-5-(trifluoromethyl)pyrimidine-2,4-diamine

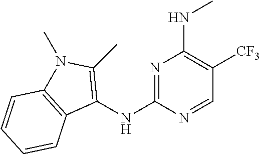 [SMILES: CNc1nc(Nc2c(C)n(C)c3ccccc23)ncc1C(F)(F)F]

### Step 1

The title compound was made from 2-methylindole following the procedure as described in Example 63, Step 1. The crude product was triturated with a mixture of MTBE and Heptanes to provide 2-methyl-3-nitro-1H-indole (Intermediate 64-a) as a brown solid (0.49 g, 37%). LCMS (−ESI) m/z 175.1 [M−H]⁻.

### Step 2

To a stirring solution of 2-methyl-3-nitro-1H-indole (Intermediate 64-a) (100 mg, 568 μmol, 1.00 eq) in anhydrous DMF (5.70 mL) cooled to 0° C., was added sodium hydride (60% dispersion in mineral oil) (25.0 mg, 624 μmol, 1.10 eq). The reaction mixture was stirred for 30 min before iodomethane (39.0 μL, 624 μmol, 1.10 eq) was added dropwise. The reaction mixture was warmed to room temperature and stirred for 3 h. The reaction mixture was partitioned between water (15 mL) and EtOAc (15 mL), and the aqueous layer was extracted with EtOAc (15 mL×2). The combined organic layers were washed with brine, dried over MgSO₄, filtered, and concentrated under reduced pressure. The crude product was purified by flash chromatography on silica gel column using 0-20% EtOAc/Hexanes as eluent, to provide 1,2-dimethyl-3-nitro-1H-indole (Intermediate 64-b) as an orange solid (67 mg, 62%). LCMS (ESI) m/z 1911 [M+H]⁺.

### Step 3

To a stirring solution of 1,2-dimethyl-3-nitro-1H-indole (Intermediate 64-b) (100 mg, 526 μmol, 1.00 eq) in THF (5.26 mL), was added Pd(OH)₂ (20 wt. %) on carbon (148 mg, 210 μmol, 0.40 eq). The reaction mixture was degassed under reduced pressure with backflow of hydrogen (3×) and stirred for 18 h under standard hydrogen atmosphere. The reaction mixture was filtered over Celite and concentrated under reduced pressure to provide 1,2-dimethyl-H-indol-3-amine (Intermediate 64-c) as a brown oil (84.2 mg, crude). LCMS (ESI) m/z 161.0 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 1,2-dimethyl-1H-indol-3-amine (Intermediate 64-c) was used in place of 1H-indazol-6-amine. The crude product was purified by flash chromatography on silica gel column using 0-30% EtOAc/Hexanes as eluent, to provide the title compound as a pale-yellow solid (26.0 mg, 29%). ¹H NMR (400 MHz, DMSO-d₆) δ ppm 2.25 (s, 3H), 2.92 (br s, 1H), 3.32 (s, 3H), 3.68 (s, 3H), 6.79 (br s, 1H), 6.93 (t, J=7.4 Hz, 1H), 7.03-7.07 (m, 1H), 7.23 (br s, 1H), 7.38 (d, J=8.2 Hz, 1H), 7.88-8.82 (br m, 1H); ¹⁹F NMR (376 MHz, DMSO-d₆) δ ppm −59.8 (s, 3F); LCMS (ESI) m/z 336.1 [M+H]⁺.

### Example 65

### Synthesis of 2-methyl-3-((4-(methylamino)-5-(trifluoromethyl)pyrimidin-2-yl)amino)pyrazolo[1,5-a]pyridine-7-carbonitrile

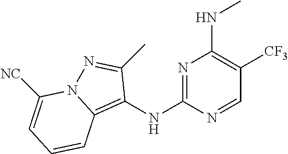 [SMILES: CNc1nc(Nc2c(C)nn3c(C#N)cccc23)ncc1C(F)(F)F]

### Step 1

To a stirring solution of tert-butyl (2-ethylpyrazolo[1,5-a]pyridin-3-yl)carbamate (Intermediate 62-c) (300 mg, 1.21 mmol, 1.00 eq) in anhydrous THF (12.1 mL) cooled to −78° C., was added n-BuLi (1.42 M in Hexane) (2.31 mL, 3.28 mmol, 2.70 eq). The reaction mixture was stirred for 1 h at −78° C. and then a solution of 1,2-diiodoethane (733 mug, 2.55 mmol, 2.10 eq) in THF (2.43 mL was added dropwise. The reaction mixture was warmed to room temperature and stirred for 1 h. The reaction mixture was diluted with EtOAc (20 mL) and a saturated solution of NH₄Cl (30 mL) was added. The aqueous layer was extracted with EtOAc (20 mL×2) and the combined organic layers were dried over MgSO₄, filtered, and concentrated under reduced pressure. The crude product was purified by flash chromatography on silica gel column using 0-20% EtOAc/Hexanes as eluent, to provide tert-butyl (7-iodo-2-methylpyrazolo[1,5-a]pyridin-3-yl)carbamate (Intermediate 65-a) as a clear oil (0.31 g, 69). LCMS (ESI) m/z 374.1 [M+H]⁺.

### Step 2

The title compound was made from tert-butyl (7-iodo-2-methylpyrazolo[1,5-a]pyridin-3-yl)carbamate (Intermediate 65-a) following the procedure as described in Example 44, Step 1, except that the reaction mixture was warmed to 140° C. under microwave irradiation and stirred for 15 min. The crude product was purified by flash chromatography on silica gel column using 0-20% EtOAc/Hexanes as eluent, to provide tert-butyl (7-cyano-2-methylpyrazolo[1,5-a]pyridin-3-yl)carbamate (Intermediate 65-b) as a yellow solid (0.11 g, 78%).

### Step 3

The title compound was made from tert-butyl (7-cyano-2-methylpyrazolo[1,5-a]pyridin-3-yl)carbamate (Intermediate 65-b) following the procedure as described in Example 4, Step 4. The volatiles were removed under reduced pressure to provide 3-amino-2-methylpyrazolo[1,5-a]pyridine-7-carbonitrile (Intermediate 65-c) as a brown oil (50.6 mg, crude). LCMS (ESI) m/z 173.1 [M+H]⁺.

### Step 4

The title compound was made from 2-chloro-N-methyl-5-(trifluoromethyl)pyrimidin-4-amine (intermediate 1-a) following the procedure as described in Example 1, Step 2, except that 3-amino-2-methylpyrazolo[1,5-a]pyridine-7-carbonitrile (Intermediate 65-c) was used ins place of 1H-indazol-6-amine. The crude product was purified by flash chromatography on silica gel column using 0-50% EtOAc/Hexanes as eluent, followed by triturated with a mixture of MTBE and Heptanes to provide the title compound as a pale-yellow powder (38.2 mg, 37%). ¹H NMR (400 MHz, DMSO-d₆) δ ppm 2.34 (s, 3H), 2.92 (br s, 1H), 3.34 (s, 3H), 6.99 (s, 1H), 7.21 (dd, J=9.0, 7.1 Hz, 1H), 7.68 (d, J=7.0 Hz, 1H), 7.74 (d, J=8.7 Hz, 1H), 7.98-9.16 (br m, 1H); ¹⁹F NMR (376 MHz, DMSO-d₆) δ ppm −60.1 (s, 3F); LCMS (ESI) m/z 348.1 [M+H]⁺.

### Example 66

### Synthesis of 2-methyl-3-((4-(methylamino)-5-(trifluoromethyl)pyrimidin-2-yl)amino)-1H-indole-7-carbonitrile

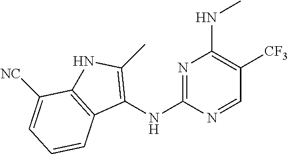 [SMILES: CNc1nc(Nc2c(C)[nH]c3c(C#N)cccc23)ncc1C(F)(F)F]

### Step 1

The title compound was made from 7-bromo-2-methyl-3-nitro-1H-indole (Intermediate 63-b) following the procedure as described in Example 44, Step 1, except that the reaction mixture was warmed to 140° C. under microwave irradiation and stirred for 30 min. The crude product was triturated with a mixture of MTBE and Heptanes to provide 2-methyl-3-nitro-1H-indole-7-carbonitrile (Intermediate 66-a) as a tan powder (0.14 g, 72%).

### Step 2

The title compound was made from 2-methyl-3-nitro-1H-indole-7-carbonitrile (Intermediate 66-a) following the procedure as described in Example 7, Step 1. The volatiles were removed under reduced pressure to provide 3-amino-2-methyl-1H-indole-7-carbonitrile (Intermediate 66-b) as a brown oil (91.1 mg, crude). LCMS (ESI) m/z 172.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 3-amino-2-methyl-1l-H-indole-7-carbonitrile (Intermediate 66-b) was used in place of 1H-indazol-6-amine. The crude product was purified by flash chromatography on silica gel column using 0-50% EtOAc/Hexanes as eluent, to provide the title compound as an off-white powder (68.0 mg, 37%). ¹H NMR (400 MHz, DMSO-d₆) δ ppm 2.29 (s, 3H), 2.92 (br s, 1H), 3.31 (s, 3H), 6.84 (br s, 1H), 7.06 (t, J=7.6 Hz, 1H), 7.47 (d, J=7.4 Hz, 1H), 7.56 (d, J=8.9 Hz, 1H), 7.93-8.91 (br m, 1H), 11.69 (s, 1H), ¹⁹F NMR (376 MHz, DMSO-d₁) δ ppm −59.8 (s, 3F); LCMS (ESI) m/z 347.1 [M+H]⁺.

### Example 67

### Synthesis of N4-methyl-N2-(1-methyl-1H-indazol-3-yl)-5-(trifluoromethyl)pyrimidine-2,4-diamine

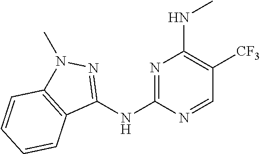 [SMILES: CNc1nc(Nc2nn(C)c3ccccc23)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 3-Amino-1-methylindazole was used in place of 1H-indazol-6-amine. The crude product was purified by reverse phase chromatography on a C18 column using 5-20% MeCN/H₂O (0.1% FA) as eluent, to provide the title compound as an off-white powder (27.0 mg, 32%). ¹H NMR (400 MHz, DMSO-d₆) δ ppm 2.66 (s, 3H), 3.96 (s, 3H), 7.01-706 (m, 2H), 7.36 (m, 1H), 7.54-7.59 (m, 2H), 8.09 (s, 1H), 9.69 (s, 1H); ¹⁹F NMR (376 MHz, DMSO-d₆) δ ppm −60.2 (s, 3F), LCMS (ESI) m/z 323.1 [M+H]⁺.

### Example 68

### Synthesis of N4-cyclopropyl-N2-(1-methyl-1H-indazol-4-yl)-5-(trifluoromethyl)pyrimidine-2,4-diamine

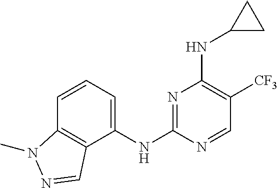 [SMILES: Cn1ncc2c(Nc3ncc(C(F)(F)F)c(NC4CC4)n3)cccc21]

### Step 1

The title compound was made from 2,4-dichloro-5-trifluoromethylpyrimidine following the procedure as described in Example 1, Step 1, except that cyclopropanamine was used in place of methylamine to provide a mixture of isomers containing 2-chloro-N-cyclopropyl-5-(trifluoromethyl)pyrimidin-4-amine (Intermediate 68-a) as a white powder (1.00 g, 47%). LCMS (ESI) m/z 238.0 [M+H]⁺.

### Step 2

The title compound was made from 2-chloro-N-cyclopropyl-5-(trifluoromethyl)pyrimidin-4-amine (Intermediate 68-a) following the procedure as described in Example 1, Step 2, except that 1-Methyl-1H-indazol-4-amine was used in place of 1H-indazol-6-amine. The crude product was purified by flash chromatography on silica gel column using 0-90% EtOAc/Hexane as eluent, to provide title compound as a white powder (100.0 mg, 68%). ¹H NMR (400 MHz, DMSO-d₃) δ ppm 0.71-0.67 (m, 2H), 0.81-0.76 (m, 2H), 2.90-2.86 (m, 1H), 4.00 (s, 3H), 7.21 (m, 2H), 7.33 (t, J=8.0 Hz, 1H), 8.25 (m, 1H), 8.46 (d, J=0.9 Hz, 1H), 9.84 (s, 1H); ¹⁹F NMR (376 MHz, DMSO-d₆) δ ppm −59.99 (s, 3F); LCMS (ESI) m/z 349.1 [M+H]⁺.

### Example 69

### Synthesis of N4-ethyl-N2-(1-methyl-1H-indazol-4-yl)-5-(trifluoromethyl)pyrimidine-2,4-diamine

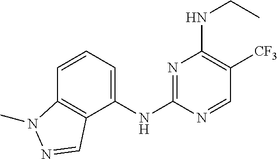 [SMILES: CCNc1nc(Nc2cccc3c2cnn3C)ncc1C(F)(F)F]

### Step 1

The title compound was made from 2,4-dichloro-5-trifluoromethylpyrimidine following the procedure as described in Example 1, Step 1, except that ethylamine was used in place of methylamine to provide a mixture of isomers containing 2-chloro-N-ethyl-5-(trifluoromethyl)pyrimidin-4-amine (Intermediate 69-a) as a white powder (0.50 g, 98%). LCMS (ESI) m/z 226.0 [M+H]⁺.

### Step 2

The title compound was made from 2-chloro-N-ethyl-5-(trifluoromethyl)pyrimidin-4-amine (Intermediate 69-a) following the procedure as described in Example 1, Step 2, except that 1-Methyl-1H-indazol-4-amine was used in place of 1H-indazol-6-amine. The crude product was purified by flash chromatography on silica gel column using 0-90% EtOAc/Hexane as eluent, to provide title compound as a white powder (40.0 mg, 27%). ¹H NMR (400 MHz, DMSO-d₆) δ ppm 1.16 (t, J=7.1 Hz, 3H), 3.49 (m, 2H), 4.00 (s, 3H), 7.18 (m, 1H), 7.23 (d, J=8.3 Hz, 1H), 7.32 (t, J=7.9 Hz, 1H), 7.88 (d, J=7.5 Hz, 1H), 8.22 (s, 1H), 8.38 (d, J=0.9 Hz, 1H), 9.74 (s, 1H); ¹⁹F NMR (376 MHz, DMSO-d₆) δ ppm −60.35 (s, 3F); LCMS (ESI) m/z 337.1 [M+H]⁺.

### Example 70

### Synthesis of 5-chloro-N4-ethyl-N2-(1-methyl-1H-indazol-4-yl)pyrimidine-2,4-diamine

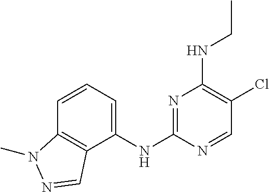 [SMILES: CCNc1nc(Nc2cccc3c2cnn3C)ncc1Cl]

### Step 1

To a stirred solution of 2,4,5-trichloropyrimidine (1.00 g, 5.45 mmol, 100 eq) in anhydrous THE (15.0 mL) was added ethylamine (5.45 mL, 10.9 mmol, 2.00 eq) dropwise at 0° C. After the reaction mixture was warmed to room temperature (RT) and stirred overnight. The reaction mixture was diluted with water and extracted with 3×25 mL of EtOAc. The combined organic layer was washed with brine, dried over anhydrous Na₂SO₄ and concentrated to dryness to obtain 2,5-dichloro-N-ethylpyrimidin-4-amine (Intermediate 70-a) as a white solid (0.91 g, 87%); LCMS (ESI) m/z 193.9 [M+H]⁺.

### Step 2

The title compound was made from 2,5-dichloro-N-ethylpyrimidin-4-amine (Intermediate 70-a) following the procedure as described in Example 1, Step 2, except that 1-Methyl-1H-indazol-4-amine was used in place of 1H-indazol-6-amine. The crude product was purified by flash chromatography on silica gel column using 0-90% EtOAc/Hexane as eluent, to provide title compound as a white powder (27.0 mg, 17%). ¹H NMR (400 MHz, DMSO-d₆) δ ppm 1.18 (t, J=7.1 Hz, 3H), 3.48-3.43 (m, 2H), 3.99 (s, 3H), 7.15 (d, J=8.3 Hz, 1H), 7.24 (m, 1H), 7.28 (t, J=8.0 Hz, 1H), 7.92 (d, J=7.7 Hz, 1H), 7.98 (s, 1H), 8.41 (d, J=0.9 Hz, 1H), 9.34 (s, 1H); LCMS (ESI) m/z 3031 [M+H]⁺.

### Example 71

### Synthesis of 5-chloro-N4-cyclopropyl-N2-(1-methyl-1H-indazol-4-yl)pyrimidine-2,4-diamine

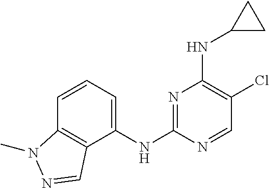 [SMILES: Cn1ncc2c(Nc3ncc(Cl)c(NC4CC4)n3)cccc21]

### Step 1

The title compound was made from 2,4,5-trichloropyrimidine following the procedure as described in Example 70, Step 1, except that cyclopropylamine was used in place of ethylamine to provide 2,5-dichloro-N-cyclopropylpyrimidin-4-amine (Intermediate 71-a) as a white solid (0.95 g, 85%); LCMS (ESI) m/z 205.9 [M+H]⁺.

### Step 2

The title compound was made from 2,5-dichloro-N-cyclopropylpyrimidin-4-amine (Intermediate 71-a) following the procedure as described in Example 1, Step 2, except that 1-Methyl-1H-indazol-4-amine was used in place of 1H-indazol-6-amine. The crude product was purified by flash chromatography on silica gel column using 0-90% EtOAc/Hexane as eluent, to provide title compound as a white powder (41.0 mg, 27%). ¹H NMR (400 MHz, DMSO-d₆) δ ppm 0.69-0.65 (m, 2H), 0.81-0.76 (m, 2H), 2.87 (m, 1H), 3.99 (s, 3H), 7.15-7.13 (m, 1H), 7.29 (t, J=8.0 Hz, 1H), 7.34 (m, 1H), 8.00 (s, 1H), 8.20 (d, J=7.7 Hz, 1H), 8.46 (d, J=0.9 Hz, 1H), 9.43 (s, 1H); LCMS (ESI) m/z 315.0 [M+H]⁺.

### Example 72

### Synthesis of N2-(1H-indazol-5-yl)-N4-methyl-5-(trifluoromethyl)pyrimidine-2,4-diamine

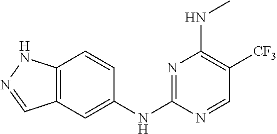 [SMILES: CNc1nc(Nc2ccc3[nH]ncc3c2)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 1H-indazol-5-amine was used in place of 1H-indazol-6-amine. The crude product was purified by flash chromatography on silica gel column using 0-90% EtOAc/Hexane as eluent, to provide title compound as a white powder (35.0 mg, 24%). ¹H NMR (400 MHz, DMSO-d₆) δ ppm 2.94 (d, J=4.3 Hz, 3H), 7.09 (s, 1H), 7.44 (d, =J=8.9 Hz, 1H), 7.60 (d, J=9.0 Hz, 1H), 7.98 (s, 1H), 8.16 (s, 1H), 8.24 (s, 1H), 9.60 (s, 1H), 12.92 (s, 1H); ¹⁹F NMR (376 MHz, DMSO-d₆) δ ppm −60.13 (s, 3F), LCMS (ESI) m/z 309.1 [M+H]⁺.

### Example 73

### Synthesis of N4-methyl-N2-(1-methyl-1H-indazol-5-yl)-5-(trifluoromethyl)pyrimidine-2,4-diamine

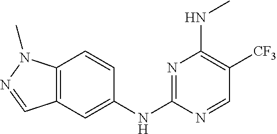 [SMILES: CNc1nc(Nc2ccc3c(cnn3C)c2)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 1-methyl-1H-indazol-5-ylamine was used in place of 1H-indazol-6-amine. The crude product was purified by flash chromatography on silica gel column using 0-90% EtOAc/Hexane as eluent, to provide title compound as a white powder (30.0 mg, 20%). 1H NMR (400 MHz, DMSO-d₆) δ ppm 2.94 (d, J=4.2 Hz, 3H), 4.01 (s, 3H), 7.12 (s, 1H), 7.55 (d, J=9.0 Hz, 1H), 7.66 (d, J=9.1 Hz, 1H), 7.95 (s, 1H), 8.17 (s, 1H), 8.22 (s, 1H), 9.63 (s, 1H); ¹⁹F NMR (376 MHz, DMSO-d₆) δ ppm −60.17 (s, 3F); LCMS (ESI) m/z 323.0 [M+H]⁺.

### Example 74

### Synthesis of N4-methyl-N2-(2-methyl-2H-indazol-5-yl)-5-(trifluoromethyl)pyrimidine-2,4-diamine

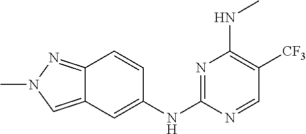 [SMILES: CNc1nc(Nc2ccc3nn(C)cc3c2)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 2-methyl-2H-indazol-5-amine was used in place of 1H-indazol-6-amine. The crude product was purified by flash chromatography on silica gel column using 0-90% EtOAc/Hexane as eluent, to provide title compound as a white powder (70.0 mg, 46%). ¹H NMR (400 MHz, DMSO-d₆) δ ppm 2.95 (d, J=3.9 Hz, 3H), 4.12 (s, 3H), 7.07 (s, 1H), 7.47 (q, J=10.2 Hz, 2H), 8.16 (d, J=3.5 Hz, 1H), 8.20 (s, 2H), 9.52 (s, 1H); ¹⁹F NMR (376 MHz, DMSO-d₆) δ ppm −60.12 (s, 3F); LCMS (ESI) m/z 323.1 [M+H]⁺.

### Example 75

### Synthesis of N2-(1H-indazol-7-yl)-N4-methyl-5-(trifluoromethyl)pyrimidine-2,4-diamine

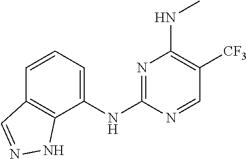 [SMILES: CNc1nc(Nc2cccc3cn[nH]c23)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 1H-indazole-7-amine was used in place of 1H-indazol-6-amine. The crude product was purified by flash chromatography on silica gel column using 0-90% EtOAc/Hexane as eluent, to provide title compound as a white powder (48.0 mg, 33%). ¹H NMR (400 MHz, DMSO-d₆) δ ppm 2.82 (d, J=4.3 Hz, 3H), 7.07 (t, J=7.8 Hz, 1H), 7.14 (d, J=5.1 Hz, 1H), 7.46 (d, J=8.0 Hz, 1H), 7.84 (d, J=7.5 Hz, 1H), 8.05 (d, J=1.4 Hz, 1H), 8.19 (s, 1H), 9.49 (s, 1H), 12.80 (s, 1H); ¹⁹F NMR (376 MHz, DMSO-d₆) δ ppm −60.33 (s, 3F); LCMS (ESI) m/z 309.0 [M+H]⁺.

### Example 76

### Synthesis of N4-methyl-N2-(1-methyl-1-indazol-7-yl)-5-(trifluoromethyl)pyrimidine-2,4-diamine

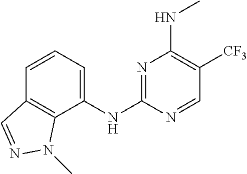 [SMILES: CNc1nc(Nc2cccc3cnn(C)c23)ncc1C(F)(F)F]

The title compound was made from 2-chloro-N-methyl-5-(trifluoromethyl)pyrimidin-4-amine (Intermediate 1-a) following the procedure as described in Example 1, Step 2, except that 1-methyl-1H-indazol-7-amine was used in place of 1H-indazol-6-amine. The crude product was purified by flash chromatography on silica gel column using 0-90% EtOAc/Hexane as eluent, to provide title compound as a white powder (100.0 mg, 66%). ¹H NMR (400 MHz, DMSO-d₆) δ ppm 2.68 (br s, 3H), 3.98 (s, 3H), 7.09-7.00 (m, 2H), 7.20 (d, J=7.3 Hz, 1H), 7.61 (d, J=8.0 Hz, 1H), 8.03 (s, 1H), 8.06 (s, 1H), 9.37 (s, 1H); ¹⁹F NMR (376 MHz, DMSO-d₆) δ ppm −60.22 (s, 3F); LCMS (ESI) m/z 323.1 [M+H]⁺.

### Example 77

### Synthesis of 3-(4-((4-(methylamino)-5-(trifluoromethyl)pyrimidin-2-yl)amino)-1H-indazol-1-yl)-1-morpholinopropan-1-one

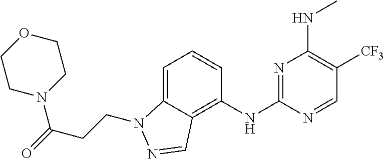 [SMILES: CNc1nc(Nc2cccc3c2cnn3CCC(=O)N2CCOCC2)ncc1C(F)(F)F]

### Step 1

To a stirring solution of morpholine (0.76 mL, 8.66 mmol, 1.10 eq) in CH₂Cl₂ (20.0 mL) cooled to 0 was added methylamine (1.32 mL, 9.45 mmol 1.20 eq) and 3-chloropropionyl chloride (0.75 mL, 7.88 mmol, 1.00 eq). After 15 min, the reaction mixture was warmed to room temperature and stirred for 18 h. The reaction mixture was diluted with CH₂Cl₂ (50 mL) and a saturated solution of NaHCO₃ (50 mL) was added. The layers were separated, and the aqueous laver was extracted with CH₂Cl₂ (50 mL×2). The combined organic layers were washed with brine (100 mL), dried over anhydrous MgSO₄, filtered, and concentrated under reduced pressure to provide 3-chloro-1-morpholinopropan-1-one (Intermediate 77-a) as a light-yellow liquid (1.35 g, crude). ¹H NMR (400 MHz, CDCl₃) δ ppm 2.79 (t, J=7.0 Hz, 2H), 3.47 (t, =4.8 Hz, 2H), 3.64-3.62 (m, 2H), 3.69-3.66 (m, 4H), 3.83 (t, J=7.0 Hz, 2H).

### Step 2

The title compound was made from 4-nitro-1H-indazole following the procedure as described in Example 31, Step 1, except that 3-chloro-1-morpholinopropan-1-one (Intermediate 77-a) was used in place of 2-bromo-2-methylpropionamide. The crude product was purified by flash chromatography on silica gel column using 0-100% EtOAc/Hexanes as eluent, to provide 1-morpholino-3-(4-nitro-1H-indazol-1-yl)propan-1-one (intermediate 77-b) as a yellow solid (0.25 g, 29%). LCMS (ESI) m/z 305.1 [M+H]⁺.

### Step 3

The title compound was made from 1-morpholino-3-(4-nitro-1H-indazol-1-yl)propan-1-one (Intermediate 77-b) following the procedure as described in Example 33, Step 2. The volatiles were removed under reduced pressure to provide 3-(4-amino-1H-indazol-1-yl)-1-morpholinopropan-1-one (Intermediate 77-c) as a pale-yellow oil (90.0 mg, crude). LCMS (ESI) m/z 275.1 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 3-(4-amino-1H-indazol-1-yl)-1-morpholinopropan-1-one (Intermediate 77-c) was used in place of 1H-indazol-6-amine. The crude product was purified by reverse phase preparative HPLC (Gemini® 5 um NX-C18 110 Å, 100×30 mm; mobile phase: [water (10 mM Ammonium Formate pH=3.8 v/v)-MeOH]; B %: 40%-100%, 16 min), to provide the title compound as an off-white solid (50.0 mg, 34%). ¹H NMR (400 MHz, DMSO-d₆) δ ppm 2.94-2.90 (m, 5H), 3.47-3.31 (m, 8H), 4.58 (t, J=6.9 Hz, 2H), 7.17 (d, J=5.0 Hz, 1H), 7.32-7.25 (m, 2H), 7.89 (d, J=7.2 Hz, 1H), 8.21 (s, 1H), 8.41 (1H, d, J=0.8 Hz), 9.75 (1H, s); ¹⁹F NMR (376 MHz, DMSO-d₆) δ ppm −60.4 (s, 3F); LCMS (ESI) m/z 450.2 [M+H]⁺.

### Example 78

### Synthesis of N4-cyclopropyl-N2-(1-(cyclopropylsulfonyl)-1H-indazol-4-yl)-5-(trifluoromethyl)pyrimidine-2,4-diamine

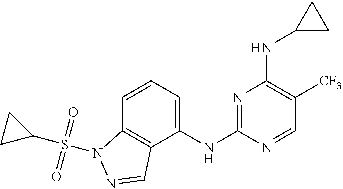 [SMILES: O=S(=O)(C1CC1)n1ncc2c(Nc3ncc(C(F)(F)F)c(NC4CC4)n3)cccc21]

The title compound was made from 2-chloro-N-cyclopropyl-5-(trifluoromethyl)pyrimidin-4-amine (Intermediate 68-a) following the procedure as described in Example 1, Step 2, except that 1-(cyclopropylsulfonyl)-1H-indazol-4-amine (Intermediate 33-c) was used in place of 1H-indazol-6-amine. The crude product was purified by flash chromatography on silica gel column using 0-50% EtOAc/Hexanes as eluent, to provide title compound as an off-white solid (24.0 mg, 15%) ¹H NMR (400 MHz, DMSO-d₆) δ ppm 0.67-0.80 (m, 4H), 1.05-1.14 (m, 2H), 1.20-1.27 (m, 2H), 2.85 (tq, J=6.9, 3.5 Hz, 1H), 3.02-3.11 (m, 1H), 7.58-7.65 (m, 1H), 7.65-7.70 (m, 1H), 8.00 (br s, 1H), 8.27 (d, J=7.5 Hz, 1H), 8.45 (s, 1H), 8.98 (s, 1H), 10.87 (br s, 1H); LCMS (ESI) m/z 439.1 [M+H]⁺.

### Example 79

### Synthesis of 5-chloro-N4-methyl-N2-(1-methyl-1H-indazol-4-yl)pyrimidine-2,4-diamine

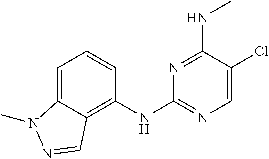 [SMILES: CNc1nc(Nc2cccc3c2cnn3C)ncc1Cl]

### Step 1

The title compound was made from 2,4,5-trichloropyrimidine following the procedure as described in Example 71, Step 1, except that methylamine was used in place of cyclopropylamine to provide 2,5-dichloro-N-methylpyrimidin-4-amine (Intermediate 79-a) as a white powder (0.85 g, 88%). LCMS (ESI) m/z 177.9 [M+H]⁺.

### Step 2

The title compound was made from 2,5-dichloro-N-methylpyrimidin-4-amine (Intermediate 79-a) following the procedure as described in Example 1, Step 2, except that 1-Methyl-1H-indazol-4-amine was used in place of 1H-indazol-6-amine. The crude product was purified by flash chromatography on silica gel column using 0-90% EtOAc/Hexane as eluent, to provide the title compound as a white powder (16.0 mg, 10%). ¹H NMR (400 MHz, DMSO-d₆) δ ppm 2.93 (d, J=4.6 Hz, 3H), 3.99 (s, 3H), 7.15 (d, J=8.3 Hz, 1H), 7.24 (m, 1H), 7.29 (t, J=8.0 Hz, 1H), 7.94 (d, J=7.7 Hz, 1H), 7.97 (s, 1H), 8.41 (d, J=0.9 Hz, 1H), 9.35 (s, 1H); LCMS (ESI) m/z 289.0 [M+H]⁺.

### Example 80

### Synthesis of 2-(4-((4-(methylamino)-5-(trifluoromethyl)pyrimidin-2-yl)amino)-7-(trifluoromethyl)-1H-indazol-1-yl)acetonitrile

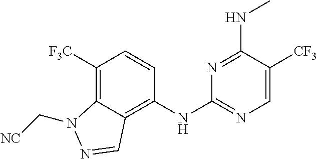 [SMILES: CNc1nc(Nc2ccc(C(F)(F)F)c3c2cnn3CC#N)ncc1C(F)(F)F]

### Step 1

The title compound was made from 4-bromo-7-(trifluoromethyl)-1H-indazole following the procedure as described in Example 16, Step 1 The crude product was purified by flash chromatography on silica gel column using 0-30% EtOAc/Hexanes as eluent, to provide 2-(4-bromo-7-(trifluoromethyl)-1H-indazol-1-yl)acetonitrile (Intermediate 80-a) as a white solid (0.18 g, 66%). ¹H NMR (400 MHz, CDCl) δ ppm 5.44 (s, 2H), 7.50 (d, J=7.9 Hz, 1H), 7.67 (d, J=7.9 Hz, 1H), 8.27 (s, 1H).

### Step 2

The title compound was made from 2-(4-bromo-7-(trifluoromethyl)-1H-indazol-1-yl)acetonitrile (Intermediate 80-a) following the procedure as described in Example 4, Step 3. The crude product was purified by flash chromatography on silica gel column using 0-50% EtOAc/Hexanes as eluent, to provide tert-butyl (1-(cyanomethyl)-7-(trifluoromethyl)-1H-indazol-4-yl)carbamate (Intermediate 80-b) as a clear oil (70.0 mg, 56%). LCMS (ESI) m/z 341.1 [M+H]⁺.

### Step 3

The title compound was made from tert-butyl (1-(cyanomethyl)-7-(trifluoromethyl)-1H-indazol-4-yl)carbamate (Intermediate 80-b) following the procedure as described in Example 4, Step 4. The volatiles were removed under reduced pressure to provide 2-(4-amino-7-(trifluoromethyl)-1H-indazol-1-yl)acetonitrile (Intermediate 80-c) as a brown oil (45.9 mg, crude). LCMS (ESL) m/z 241.2 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 2-(4-amino-7-(trifluoromethyl)-1H-indazol-1-yl)acetonitrile (Intermediate 80-c) was used in place of 1H-indazol-6-amine. The crude product was purified by flash chromatography on silica gel column using 0-40% EtOAc/Hexanes as eluent, to provide the title compound as a white solid (7.0 mg, 9.0%). ¹H NMR, (400 MHz, DMSO-d₆) δ ppm 2.97 (d, J=4.4 Hz, 3H), 5.61 (s, 2H), 7.36-7.39 (m, 1H), 7.89 (d, J=8.5 Hz, 1H), 8.30-8.32 (m, 2H), 8.93 (s, 1H), 10.29 (s, 1H), ¹⁹F NMR (376 MHz, DMSO-d₆) δ ppm −54.2 (s, 3F), −60.9 (s, 3F); LCMS (ESI) m/z 416.2 [M+H]⁺.

### Example 81

### Synthesis of N4-methyl-N2-(1-methyl-7-(1-methyl-1,2,3,6-tetrahydropyridin-4-yl)-1H-indazol-4-yl)-5-(trifluoromethyl)pyrimidine-2,4-diamine

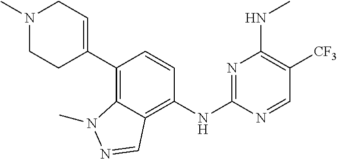 [SMILES: CNc1nc(Nc2ccc(C3=CCN(C)CC3)c3c2cnn3C)ncc1C(F)(F)F]

The title compound was made from N2-(7-bromo-1-methyl-1H-indazol-4-yl)-N4-methyl-5-(trifluoromethyl)pyrimidine-2,4-diamine (Intermediate 58-a) following the procedure as described in Example 43, Step 2, except that 1-methyl-1,2,3,6-tetrahydropyridine-4-boronic acid pinacol ester was used in place of isopropenylboronic acid pinacol ester, dioxane was used in place of DMF, and the reaction mixture was stirred at 90° C. for 7 h. The crude product was purified by reverse phase chromatography on a C18 column using 5-15% MeCN/H₂O (0.1% FA) as eluent, to provide the title compound as a yellow solid (39.0 mg, 94%), ¹H NMR (400 MHz, DMSO-d₆) δ ppm 2.31 (s, 3H), 2.42 (s, 2H), 2.61 (d, J=5.6 Hz, 2H), 2.93 (d, J=4.4 Hz, 3H), 3.04 (s, 2H), 4.00 s, 3H), 5.69-5.67 (m, 1H), 7.01 (d, J=7.8 Hz, 1H), 717-7.14 (m, 1H), 7.84 (d, J=7.8 Hz, 1H), 8.20 (d, J=1.0 Hz, 1H), 8.40 (s, 1H), 9.71 (s, 1H); ¹⁹F NMR (376 MHz, DMSO-d₆) δ ppm −60.4 (s, 3F); LCMS (ESI) m/z 418.3 [M+H]⁺.

### Example 82

### Synthesis of N4-methyl-N2-(1-methyl-7-(1-methylpiperidin-4-yl)-1H-indazol-4-yl)-5-(trifluoromethyl)pyrimidine-2,4-diamine

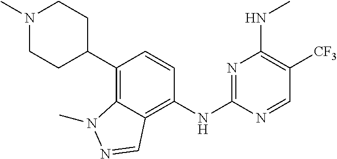 [SMILES: CNc1nc(Nc2ccc(C3CCN(C)CC3)c3c2cnn3C)ncc1C(F)(F)F]

To a stirring solution of N4-methyl-N2-(1-methyl-7-(1-methyl-1,2,3,6-tetrahydropyridin-4-yl)-1H-indazol-4-yl)-5-(trifluoromethyl)pyrimidine-2,4-diamine (Example 81) (36 mg, 86.2 μmol, 1.00 eq) in EtOH (345 mL), was added Pd(OH)₂ (20 wt. %) on carbon (60.6 mg, 86.2 μmol, 1.00 eq). The reaction mixture was degassed under reduced pressure with backflow of hydrogen (3×) and stirred for 3 days under standard hydrogen atmosphere. The reaction mixture was filtered over Celite and the volatiles removed under reduced pressure. The crude product was purified by reverse phase chromatography on a C18 column using 3-12% MeCN/H₂O (0.1% FA) as eluent, to provide the title compound as a white powder (17.0 mg, 47%). ¹H NMR (400 MHz, DMSO-d₆) δ ppm 1.86-1.72 (m, 4H), 2.14-2.08 (m, 2H), 2.24 (s, 3H), 2.92-2.91 (m, 5H), 3.28-3.22 (m, 1H), 4.22 (s, 3H), 7.16-7.11 (m, 2H), 7.80 (d, J=8.0 Hz, 1H), 8.18 (s, 1H), 8.33 (s, 1H), 9.60 (s, 1H); ¹⁹F NMR (376 MHz, DMSO-d₆) δ ppm −60.3 (s, 3F), LCMS (ESI) m/z 420.3 [M+H]⁺.

### Example 83

### Synthesis of 2-(7-(3,6-dihydro-2H-pyran-4-yl)-4-((4-(methylamino)-5-(trifluoromethyl)pyrimidin-2-yl)amino)-1H-indazol-1-yl)acetonitrile

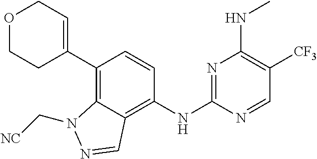 [SMILES: CNc1nc(Nc2ccc(C3=CCOCC3)c3c2cnn3CC#N)ncc1C(F)(F)F]

### Step 1

The title compound was made from 2-(4-((4-(methylamino)-5-(trifluoromethyl)pyrimidin-2-yl)amino)-1H-indazol-1-yl)acetonitrile (Example 29) following the procedure as described in Example 56, Step 1, except that the reaction mixture was warmed to 40° C. and stirred for 10 min. The crude product was purified by flash chromatography on silica gel column using 0-50% EtOAc/1-Hexanes as eluent, to provide 2-(7-bromo-4-((4-(methylamino)-5-(trifluoromethyl)pyrimidin-2-yl)amino)-1H-indazol-1-yl)acetonitrile (Intermediate 83-a) as a white solid (0.47 g, 95%). ¹H NMR (400 MHz, DMSO-d₆) δ ppm 2.92 (d, J=4.4 Hz, 3H), 5.93 (s, 2H), 7.27-7.24 (m, 1H), 7.66 (d, J=84.4 Hz, 1H), 7.97 (d, J=84.4 Hz, 1H), 8.25 (s, 1H), 8.69 (s, 1H), 9.99 (s, 1H). LCMS (ESI) m/z 428.0 [M+H]⁺.

### Step 2

The title compound was made from 2-(7-bromo-4-((4-(methylamino)-5-(trifluoromethyl)pyrimidin-2-yl)amino)-1H-indazol-1-yl)acetonitrile (Intermediate 83-a) following the procedure as described in Example 43, Step 2, except that 3,6-dihydro-2H-pyran-4-boronic acid pinacol ester was used in place of isopropenylboronic acid pinacol ester and the reaction mixture was stirred at 90° C. for 5 h. The crude product was purified by flash chromatography on silica gel column using 0-55% EtOAc/Hexanes as eluent, to provide the title compound as a white solid (70.0 trig, 69%). ¹H NMR (400 MHz, DMSO-d₆) δ ppm 2.54-2.51 (m, 2H), 3.00 (d, J=4.4 Hz, 3H), 3.99 (t, J=5.4 Hz, 2H), 4.37-4.35 (M, 2H), 5.62 (s, 2H), 5.95-5.93 (m, 1H), 7.30-7.26 (m, 2H), 8.04 (d, J=8.0 Hz, 1H), 8.29 (d, J=1.0 Hz, 1H), 8.67 (s, 1H), 9.96 (s, 1H); ¹⁹F NMR (376 MHz, DMSO-d₆) δ ppm −60.5 (s, 3F); LCMS (ESI) m/z 430.2 [M+H]⁺.

### Example 84

### Synthesis of 2-(4-((4-(methylamino)-5-(trifluoromethyl)pyrimidin-2-yl)amino)-7-(trifluoromethoxy)-1H-indazol-1-yl)acetonitrile

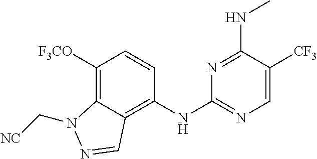 [SMILES: CNc1nc(Nc2ccc(OC(F)(F)F)c3c2cnn3CC#N)ncc1C(F)(F)F]

### Step 1

To a stirring solution of 6-Bromo-2-fluoro-3-(trifluoromethoxy)benzaldehyde (300 mg, 993 μmol, 1.00 eq) in pyridine (0.38 mL), was added hydrazine monohydrate (482 μL, 9.93 mmol, 10.0 eq) and 4-dimethylaminopyridine (12.4 mg, 99.3 μmol, 0.10 eq). The reaction mixture was warmed to 100° C. and stirred for 24 h under argon atmosphere. The reaction mixture was cooled to room temperature, poured into water (10 mL), and extracted with EtOAc (10 ml×3). The combined organic layers were washed with brine (20 mL), dried over anhydrous MgSO₄, filtered, and the volatiles removed under reduced pressure. The crude product was purified by flash chromatography on silica gel column using 0-30% EtOAc/Hexanes as eluent, to provide 4-bromo-7-(trifluoromethoxy)-1H-indazole (Intermediate 84-a) as a white solid (0.20 g, 72%). LCMS (ESI) m/z 282.9 [M+H]⁺.

### Step 2

The title compound was made from 4-bromo-7-(trifluoromethoxy)-1H-indazole (Intermediate 84-a) following the procedure as described in Example 16, Step 1, The crude product was purified by flash chromatography on silica gel column using 0-20% EtOAc/Hexanes as eluent, to provide 2-(4-bromo-7-(trifluoromethoxy)-1H-imidazol-1-yl)acetonitrile (Intermediate 84-b) as a white solid (0.18 g, 55%). ¹H NMR (400 MHz, CHCl₃-d) δ ppm 5.38 (s, 2H), 7.15 (dq, J=8.2, 1.8 Hz, 1H), 7.30 (d, J=8.2 Hz, 1H), 8.06-8.07 (m, 1H).

### Step 3

The title compound was made from 2-(4-bromo-7-(trifluoromethoxy)-H-indazol-1-yl)acetonitrile (Intermediate 84-b) following the procedure as described in Example 4, Step 3. The crude product was purified by flash chromatography on silica gel column using 0-50% EtOAc/Hexanes as eluent, to provide tert-butyl (1-(cyanomethyl)-7-(trifluoromethoxy)-1H-indazol-4-yl)carbamate (Intermediate 84-c) as a clear oil (63.0 mg, 31%). LCMS (ESI) m/z 357.1 [M+H]⁺.

### Step 4

The title compound was made from tert-butyl (1-(cyanomethyl)-7-(trifluoromethoxy)-1H-indazol-4-yl)carbamate (Intermediate 84-c) following the procedure as described in Example 4, Step 4. The volatiles were removed under reduced pressure to provide 2-(4-amino-7-(trifluoromethoxy)-1H-indazol-1-yl)acetonitrile (Intermediate 84-d) as a brown oil (45.0 mg, crude). LCMS (ESI) m/z 257.0 [M+H]⁺.

### Step 5

The title compound was made from 2-chloro-N-methyl-5-(trifluoromethyl)pyrimidin-4-amine (Intermediate 1-a) following the procedure as described in Example 1, Step 2, except that 2-(4-amino-7-(trifluoromethoxy)-1H-indazol-1-yl)acetonitrile (Intermediate 84-d) was used in place of 1H-indazol-6-amine. The crude product was purified by flash chromatography on silica gel column using 0-40% EtOAc/Hexanes as eluent, to provide the title compound as a white solid (34.0 mg, 45%). ¹H NMR (400 MHz, DMSO-d₆) δ ppm 2.94 (d, J=4.4 Hz, 3H), 5.70 (s, 2H), 7.28-7.25 (m, 1H), 7.48 (dq, J=8.6, 1.7 Hz, 1H), 8.04 (d, J=8.6 Hz, 1H), 8.25 (d, J=1.0 Hz, 1H), 8.70 (s, 1H), 10.05 (s, 1H); ¹⁹F NMR (376 MHz, DMSO-d₆) δ ppm −56.7 (s, 3F), −60.7 (s, 3F); LCMS (ESI) m/z 132.2 [M+H]⁺.

### Example 85

### Synthesis of 2-(7-fluoro-4-((4-(methylamino)-5-(trifluoromethyl)pyrimidin-2-yl)amino)-1H-indazol-1-yl)acetonitrile

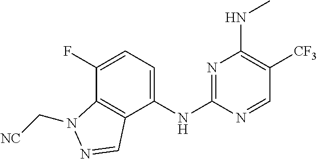 [SMILES: CNc1nc(Nc2ccc(F)c3c2cnn3CC#N)ncc1C(F)(F)F]

### Step 1

The title compound was made from 4-bromo-7-fluoro-1H-indazole following the procedure as described in Example 16, Step 1. The crude product was purified by flash chromatography on silica gel column using 0-40% EtOAc/Hexanes as eluent, to provide 2-(4-bromo-7-fluoro-1H-indazol-1-yl)acetonitrile (Intermediate 85-a) as a white solid (245.0 mg, 83%). ¹H NMR (400 MHz, DMSO-d₆) δ ppm 5.83 (s, 2H), 7.34 (dd, J=11.4, 8.2 Hz, 1H), 7.44 (dd, J=8.2, 3.6 Hz, 1H), 8.28 (d, J=2.3 Hz, 1H).

### Step 2

The title compound was made from 2-(4-bromo-7-fluoro-1H-indazol-1-yl)acetonitrile (Intermediate 85-a) following the procedure as described in Example 4, Step 3. The crude product was purified by flash chromatography on silica gel column using 0-50% EtOAc/Hexanes as eluent, to provide (1-(cyanomethyl)-7-fluoro-1H-indazol-4-yl)carbamate (Intermediate 85-b as a yellow solid (170.0 mg, 65%). LCMS (ESI) m/z 291.2 [M+H]⁺.

Step 3

The title compound was made from tert-butyl (1-(cyanomethyl)-7-fluoro-1H-indazol-4-yl)carbamate (Intermediate 85-b) following the procedure as described in Example 4, Step 4. The volatiles were removed under reduced pressure to provide 2-(4-amino-7-fluoro-1H-indazol-1-yl)acetonitrile (Intermediate 85-c) as a brown oil (105 mg, crude). LCMS (ESI) m/z 191.1 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 2-(4-amino-7-fluoro-1H-indazol-1-yl)acetonitrile (Intermediate 85-c) was used in place of 1H-indazol-6-amine. The crude product was purified by reverse phase preparative HPLC (Gemini® 5 um NX-C18 110 Å, 100×30 mm; mobile phase: [water (10 mM Ammonium Formate pH=3.8 v/v)-MeCN]; B %: 35%-100%, 16 min), to provide the title compound as a white solid (15.0 mg, 11%). ¹H NMR (400 MHz, DMSO-d₆) δ ppm 2.89 (d, J=4.4 Hz, 3H), 5.76 (s, 2H), 7.18 (m, 1H), 7.29 (dd, J=11.5, 8.5 Hz, 1H), 7.81 (dd, J=8.6, 3.5 Hz, 1H), 8.21 (s, 1H), 8.55 (d, J=2.4 Hz, 1H), 9.88 (s, 1H); ¹⁹F NMR (376 MHz, DMSO-d₆) δ ppm −60.5 (s, 3F), −142.3 (s, 1F); LCMS (ESI) m/z 366.1 [M+H]⁺.

### Example 86

### Synthesis of N2-(7-(3,6-dihydro-2H-pyran-4-yl)-1-methyl-11H-indazol-4-yl)-N4-methyl-5-(trifluoromethyl)pyrimidine-2,4-diamine

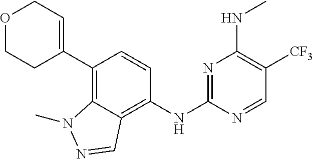 [SMILES: CNc1nc(Nc2ccc(C3=CCOCC3)c3c2cnn3C)ncc1C(F)(F)F]

The title compound was made from N2-(7-bromo-1-methyl-1H-indazol-4-yl)-N4-methyl-5-(trifluoromethyl)pyrimidine-2,4-diamine (Intermediate 58-a) following the procedure as described in Example 43, Step 2, except that 3,6-dihydro-2H-pyran-4-boronic acid pinacol ester was used in place of isopropenylboronic acid pinacol ester and the reaction mixture was stirred at 90° C. for 7 h. The crude product was purified by reverse phase chromatography on a C18 column using 5-40% MeCN/20 (0.1% FA) as eluent, to provide the title compound as a white solid (44.0 mg, 44%). ¹H NMR (400 MHz, DMSO-d₆) δ ppm 2.42-2.39 (m, 2H), 2.93 (d, J=4.4 Hz, 3H), 3.88 (t, J=5.4 Hz, 2H), 4.02 (s, 3H), 4.25 (q, J=2.7 Hz, 2H), 5.80-5.78 (m, 1H), 7.05 (d, J=7.8 Hz, 1H), 7.18-7.15 (m, 1H), 7.87 (d, J=7.9 Hz, 1H), 8.21 (s, 1H), 8.41 (s, 1H), 9.73 (s, 1H); ¹⁹F NMR (376 MHz, DMSO-d₆) δ ppm −60.4 (s, 3F); LCMS (ESI) m/z 405.1 [M+H]⁺.

### Example 87

### Synthesis of 2-(7-(1-methyl-1,2,3,6-tetrahydropyridin-4-yl)-4-((4-(methylamino)-5-(trifluoromethyl)pyrimidin-2-yl)amino)-11H-indazol-1-yl)acetonitrile

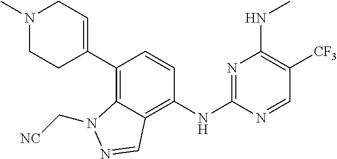 [SMILES: CNc1nc(Nc2ccc(C3=CCN(C)CC3)c3c2cnn3CC#N)ncc1C(F)(F)F]

The title compound was made from 2-(7-bromo-4-((4-(methylamino)-5-(trifluoromethyl)pyrimidin-2-yl)amino)-1H-indazol-1-yl)acetonitrile (Intermediate 83-a) following the procedure as described in Example 43, Step 2, except that 1-methyl-1,2,3,6-tetrahydropyridine-4-boronic acid pinacol ester was used in place of isopropenylboronic acid pinacol ester and the reaction mixture was stirred at 90° C. for 6 h. The crude product was purified by reverse phase chromatography on a C18 column using 5-15% MeCN/H₂O (0.1% FA) as eluent, to provide the title compound as a pale-yellow solid (78.8 mg, 76%). ¹H NMR (400 MHz, DMSO-d₆) δ ppm 2.34 (s, 3H), 2.48-2.47 (m, 2H), 2.66 (t, J=5.5 Hz, 2H), 2.93 (d, J=4.4 Hz, 3H), 3.10-3.08 (m, 2H), 5.53 (s, 2H), 5.78-5.76 (m, 1H), 7.21-7.17 (m, 2H), 7.95 (d, =J=8.0 Hz, 1H), 8.22 (s, 1H), 8.59 (s, 1H), 9.87 (s, 1H); ¹⁹F NMR (376 MHz, DMSO-d₆) δ ppm −60.5 (s, 3F); LCMS (ESI) m/z 443.3 [M+H]⁺.

### Example 88

### Synthesis of N4-methyl-N2-(1-methyl-7-(tetrahydro-2H-pyran-4-yl)-1H-indazol-4-yl)-5-(trifluoromethyl)pyrimidine-2,4-diamine

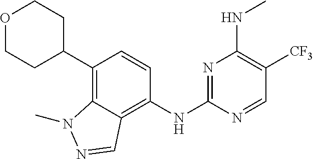 [SMILES: CNc1nc(Nc2ccc(C3CCOCC3)c3c2cnn3C)ncc1C(F)(F)F]

The title compound was made from N2-(7-(3,6-dihydro-2H-pyran-4-yl)-1-methyl-1H-indazol-4-yl)-N4-methyl-5-(trifluoromethyl)pyrimidine-2,4-diamine (Example 86) following the procedure as described in Example 82, Step 1, except that a 4:1 mixture of EtOH and NMP was used and the reaction mixture was stirred for 2 days. The crude product was purified by reverse phase chromatography on a C18 column using 5-35% MeCN/H₂O (0.1% FA) as eluent, to provide the title compound as a white powder (14.3 mg, 47%). ¹H NMR (400 MHz, DMSO-d₆) δ ppm 1.81-1.76 (m, 4H), 2.93 (d, J=4.4 Hz, 3H), 3.61-3.53 (m, 3H), 4.00-3.96 (m, 2H), 4.25 (s, 3H), 7.15-7.12 (m, 1H), 7.18 (d, J=8.0 Hz, 1H), 7.83 (d, J=8.0 Hz, 1H), 8.19 (s, 1H), 8.34 (s, 1H), 9.61 (s, 1H); ¹⁹F NMR (376 MHz, DMSO-d₆) δ ppm −60.4 (s, 3F); LCMS (ESI) m/z 407.3 [M+H]⁺.

### Example 82

### Synthesis of 2-(4-((4-(methylamino)-5-(trifluoromethyl)pyrimidin-2-yl)amino)-7-(tetrahydro-2H-pyran-4-yl)-1H-indazol-1-yl)acetonitrile

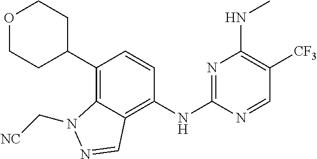 [SMILES: CNc1nc(Nc2ccc(C3CCOCC3)c3c2cnn3CC#N)ncc1C(F)(F)F]

To a stirring solution of 2-(7-bromo-4-((4-(methylamino)-5-(trifluoromethyl)pyrimidin-2-yl)amino)-1H-indazol-1-yl)acetonitrile (Intermediate 83-a) (60 mg, 141 μmol, 1.00 eq) in anhydrous DMA (0.70 mL) sparged with argon, was added 4-bromo-tetrahydropyran (47.5 μL, 422 μmol, 3.00 eq), manganese (15.5 mg, 282 μmol, 2.00 eq), pyridine (11.4 μL, 141 μmol, 1.00 eq), potassium iodide (23.6 mg, 141 μmol, 1.00 eq), and (4,4′-dtbbpy)NiCl₂ (17.7 mg, 42.2 μmol, 0.30 eq). The reaction mixture was degassed under reduced pressure with backflow of argon (3×), warmed to 80° C., and stirred for 18 h. The reaction mixture was cooled to room temperature, filtered over Celite, and concentrated under reduced pressure. The crude residue was diluted with CH₂Cl₂ (10 mL) and consecutively washed with a saturated solution of NaHCO₃ (10 mL) and brine (10 mL). The organic layer was dried over MgSO₄, filtered, and concentrated under reduced pressure. The crude product was purified by flash chromatography on silica gel column using 0-50% EtOAc/Hexanes as eluent, followed by reverse phase preparative HPLC (Gemini® 5 um NX-C18 110 Å, 100×30 mm; mobile phase: [water (10 mM Ammonium Bicarbonate pH=10.0 v/v)-MeCN]; B %: 30%-100%, 16 min) to provide the title compound as a white powder (4.0 mg, 6.6%). ¹H NMR (400 MHz, DMSO-d₆) δ ppm 1.81 (m, 4H), 2.92 (d, J=4.3 Hz, 3H), 3.47-3.40 (m, 1H), 3.65-3.59 (m, 2H), 3.99-3.95 (m, 2H), 5.84 (s, 2H), 7.18-7.15 (m, 1H), 7.34 (d, J=8.1 Hz, 1H), 7.92 (d, J=8.1 Hz, 1H), 8.20 (s, 1H), 8.54 (s, 1H), 9.76 (s, 1H) ¹⁹F NMR (376 MHz, DMSO-d₆) δ ppm −60.4 (s, 3F); LCMS (ESI) m/z 432.2 [M+H]⁺.

### Synthesis of 2-(4-((4-(methylamino)-5-(trifluoromethyl)pyrimidin-2-yl)amino)-6-(trifluoromethyl)-1H-indazol-1-yl)acetonitrile

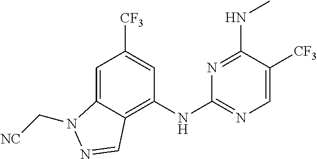 [SMILES: CNc1nc(Nc2cc(C(F)(F)F)cc3c2cnn3CC#N)ncc1C(F)(F)F]

### Step 1

The title compound was made from 4-bromo-6-(trifluoromethyl)-1H-indazole following the procedure as described in Example 16, Step 1. The crude product was purified by flash chromatography on silica gel column using 0-50% EtOAc/Hexanes as eluent, to provide 2-(4-bromo-6-(trifluoromethyl)-1H-indazol-1-yl)acetonitrile (Intermediate 90-a) as a clear oil (0.23 g, 81%). ¹H NMR (400 MHz, CHCl₃-d) δ ppm 5.34 (s, 2H), 7.64 (s, 1H), 7.73 (s, 1H), 8.17 (d, J=1.0 Hz, 1H).

### Step 2

The title compound was made from 2-(4-bromo-6-(trifluoromethyl)-1H-indazol-1-yl)acetonitrile (Intermediate 90-a) following the procedure as described in Example 4, Step 3. The crude product was purified by flash chromatography on silica gel column using 0-50% EtOAc/Hexanes as eluent, to provide tert-butyl (1-(cyanomethyl)-6-(trifluoromethyl)-1H-indazol-4-yl)carbamate (intermediate 90-b) as a clear oil (0.26 g, 99%). LCMS (ESI) m/z 341.1 [M+H]⁺.

### Step 3

The title compound was made from tert-butyl (1-(cyanomethyl)-6-(trifluoromethyl)-1H-indazol-4-yl)carbamate (intermediate 90-b) following the procedure as described in Example 4, Step 4. The volatiles were removed under reduced pressure to provide 2-(4-amino-6-(trifluoromethyl)-1H-indazol-1-yl)acetonitrile (Intermediate 90-c) as a brown oil (70.6 mg, crude). LCMS (ESI) m/z 241.1 [M+H]⁺.

### Step 4

The title compound was made from 2-chloro-N-methyl-5-(trifluoromethyl)pyrimidin-4-amine (Intermediate 1-a) following the procedure as described in Example 1, Step 2, except that 2-(4-amino-6-(trifluoromethyl)-1H-indazol-1-yl)acetonitrile (intermediate 90-c) was used in place of 1H-indazol-6-amine. The crude product was purified by flash chromatography on silica gel column using 0-40% EtOAc/Hexanes as eluent, to provide the title compound as a white solid (27.0 mg, 22%) ¹H NMR (400 MHz, DMSO-d₆) δ ppm 2.97 (d, J=4.3 Hz, 3H), 5.88 (s, 2H), 7.37-7.40 (m, 1H), 7.92 (s, 1H), 8.31 (s, 1H), 8.64 (s, 1H), 8.83 (s, 1H), 10.33 (s, 1H); ¹⁹F NMR (376 MHz, DMSO-d₆) δ ppm −60.4 (s, 3F), −60.8 (s, 3F); LCMS (ESI) m/z 416.1 [M+H]⁺.

### Example 91

### Synthesis of 2-(6-fluoro-4-((4-(methylamino)-5-(trifluoromethyl)pyrimidin-2-yl)amino)-1H-indazol-1-yl)acetonitrile

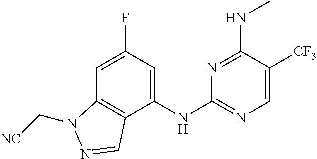 [SMILES: CNc1nc(Nc2cc(F)cc3c2cnn3CC#N)ncc1C(F)(F)F]

### Step 1

The title compound was made from 6-fluoro-4-nitro-1H-indazole following the procedure as described in Example 16, Step 1 The crude product was purified by flash chromatography on silica gel column using 0-50% EtOAc/Hexanes as eluent, to provide 2-(6-fluoro-4-nitro-1H-indazol-1-yl)acetonitrile (Intermediate 91-a) as a yellow solid (90.0 mg, 74%). LCMS (ESI) m/z 219.0 [M−H]⁻.

### Step 2

The title compound was made from 2-(6-fluoro-4-nitro-1H-indazol-1-yl)acetonitrile (Intermediate 91-a) following the procedure as described in Example 7, Step 1. The volatiles were removed under reduced pressure to provide 2-(4-amino-6-fluoro-1H-indazol-1-yl)acetonitrile (Intermediate 91-b) as a yellow solid (73.0 mg, crude). LCMS (ESI) m/z 191.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 2-(4-amino-6-fluoro-1H-indazol-1-yl)acetonitrile (Intermediate 91-b) was used in place of 1H-indazol-6-amine. The crude product was purified by flash chromatography on silica gel column using 0-50% EtOAc/Hexanes as eluent, to provide the title compound as a pale-yellow solid (60.0 mg, 43%). ¹H NMR (400 MHz, DMSO-d₆) δ ppm 2.98 (d, J=4.4 Hz, 3H), 5.72 (s, 2H), 7.28-7.25 (m, 1H), 7.35 (d, J=5.0 Hz, 1H), 8.11 (dd, J=13.2, 2.1 Hz, 1H), 8.29 (d, J=1.0 Hz, 1H), 869 (d, J=1.0 Hz, 1H), 10.18 (s, 1H); ¹⁹F NMR (376 MHz, DMSO-d₆) δ ppm −60.5 (s, 3F), −111.3 (br s, 1F); LCMS (ESI) m/z 366.1 [M+H]⁺.

### Synthesis of 2-(6-chloro-4-((4-(methylamino)-5-(trifluoromethyl)pyrimidin-2-yl)amino)-1H-indazol-1-yl)acetonitrile

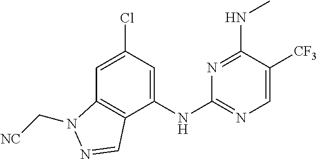 [SMILES: CNc1nc(Nc2cc(Cl)cc3c2cnn3CC#N)ncc1C(F)(F)F]

### Step 1

The title compound was made from 6-chloro-4-nitro-1H-indazole and bromoacetonitrile following the procedure as described in Example 16, Step 1 The crude product was purified by flash chromatography on silica gel column using 0-50% EtOAc/Hexanes as eluent, to provide 2-(6-chloro-4-nitro-1H-indazol-1-yl)acetonitrile (Intermediate 92-a) as a yellow solid (185.0 mg, 77%). ¹H NMR (400 MHz, DMSO-d₆) δ ppm 5.93 (s, 2H), 8.23 (d, J=1.6 Hz, 1H), 8.62 (dd, J=1.5, 1.0 Hz, 1H), 8.69 (d, J=1.0 Hz, 1H).

### Step 2

The title compound was made from 2-(6-chloro-4-nitro-1H-indazol-1-yl)acetonitrile (Intermediate 92-a) following the procedure as described in Example 7, Step 1. The volatiles were removed under reduced pressure to provide 2-(4-amino-6-chloro-1H-indazol-1-yl)acetonitrile (Intermediate 92-b) as a yellow solid (80.0 mg, crude). LCMS (EST) m/z 207.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 2-(4-amino-6-chloro-1H-indazol-1-yl)acetonitrile (Intermediate 92-b) was used in place of 1H-indazol-6-amine. The crude product was purified by flash chromatography on silica gel column using 0-50% EtOAc/Hexanes as eluent, to provide the title compound as a pale-yellow solid (110.0 mg, 74%). ¹H NMR (400 MHz, DMSO-d₆) δ ppm 2.98 (d, J=4.4 Hz, 3H), 5.76 (s, 2H), 7.35 (br s, 1H), 7.58 (s, 1H), 8.30-8.29 (m, 2H), 8.68 (d, J=0.9 Hz, 1H), 10.17 (s, 1H); ¹⁹F NMR (376 MHz, DMSO-d₆) δ ppm −60.8 (s, 3F); LCMS (ESI) m/z 382.2 [M+H]⁺.

### Example 93

### Synthesis of 2-(4-(4-(cyclopropylamino)-5-(trifluoromethyl)pyrimidin-2-yl)amino)-1H-indazol-1-yl)acetonitrile

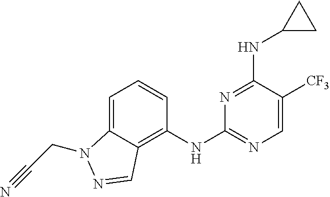 [SMILES: N#CCn1ncc2c(Nc3ncc(C(F)(F)F)c(NC4CC4)n3)cccc21]

The title compound was made from 2-chloro-N-cyclopropyl-5-(trifluoromethyl)pyrimidin-4-amine (Intermediate 68-a) following the procedure as described in Example 1, Step 2, except that 2-(4-amino-1H-indazol-1-yl)acetonitrile (Intermediate 30-b) was used in place of 1H-indazol-6-amine. The crude product was purified by flash chromatography on silica gel column using 0-90% EtOAc/Hexane as eluent, to provide the title compound as a white powder (40.0 mg, 36%). ¹H NMR (400 MHz, DMSO-d₆) δ ppm 0.71-0.67 (m, 2H), 0.78-0.75 (m, 2H), 2.87 (m, 1H), 5.76 (s, 2H), 7.24 (s, 1H), 7.37 (d, J=8.3 Hz, 1H), 7.44 (t, J=7.9 Hz, 1H), 8.29-8.26 (m, 2H), 8.63 (d, J=0.9 Hz, 1H), 9.98 (s, 1H); ¹⁹F NMR (376 MHz, DMSO-d₆) δ ppm −60.11 (s, 3F), LCMS (ESI) m/z 374.1 [M+H]⁺.

### Example 94

### Synthesis of N2-(1-(2,2-difluoroethyl)-1H-indazol-4-yl)-N4-methyl-5-(trifluoromethyl)pyrimidine-2,4-diamine

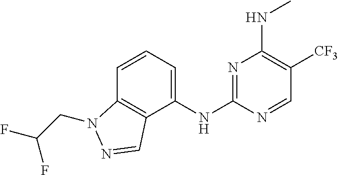 [SMILES: CNc1nc(Nc2cccc3c2cnn3CC(F)F)ncc1C(F)(F)F]

### Step 1

The title compound was made from 4-nitro-1H-indazole following the procedure as described in Example 16, Step 1, except that 2-bromo-1,1-difluoroethane was used in place of bromoacetonitrile. The crude product was purified by flash chromatography on silica gel column using 0-40% EtOAc/Hexanes as eluent, to provide 1-(2,2-difluoroethyl)-4-nitro-1H-indazole (Intermediate 94-a) as a pale-yellow solid (70.0 mg, 50%). ¹H NMR (400 MHz, DMSO-d₆) δ ppm 5.12 (td, J=15.5, 3.4 Hz, 2H), 6.49 (tt, J=54.5, 3.4 Hz, 1H), 7.71 (dd, J=8.5, 7.7 Hz, 1H), 8.21 (dd, 7.7, 0.6 Hz, 1H), 8.32 (d, J=8.5 Hz, 1H), 8.61 (d, J=0.9 Hz, 1H).

### Step 2

The title compound was made from 1-(2,2-difluoroethyl)-4-nitro-1H-indazole (Intermediate 94-a) following the procedure as described in Example 33, Step 2. The volatiles were removed under reduced pressure to provide 1-(2,2-difluoroethyl)-1H-indazol-4-amine (Intermediate 94-b) as a pale-yellow oil (43.0 mg, crude). LCMS (ESI) m/z 198.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 1-(2,2-difluoroethyl)-1H-indazol-4-amine (Intermediate 94-b) was used in place of 1H-indazol-6-amine. The crude product was purified by flash chromatography on silica gel column using 0-50% EtOAc/Hexanes as eluent, to provide the title compound as a white solid (60.0 mg, 64%). ¹H NMR (400 MHz, DMSO-d₆) δ ppm 2.93 (d, J=4.4 Hz, 3H), 4.88 (td, J=15.2, 3.7 Hz, 2H), 6.54-6.27 (m, 1H), 7.18 (d, J=5.1 Hz, 1H), 7.38-7.32 (m, 2H), 7.93 (dd, J=6.7, 1.7 Hz, 1H), 8.22 (d, J=1.0 Hz, 1H), 8.49 (d, J=0.7 Hz, 1H), 9.82 (s, 1H); ¹⁹F NMR (376 MHz, DMSO-d₆) δ ppm −60.6 (s, 3F), −121.9 (m, 2F); LCMS (ESI) m/z 373.1 [M+H]⁺.

### Example 95

### Synthesis of 2-(4-((4-(cyclopentylamino)-5-(trifluoromethyl)pyrimidin-2-yl)amino)-1H-indazol-1-yl)acetonitrile

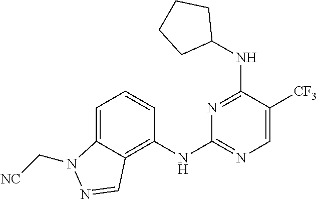 [SMILES: N#CCn1ncc2c(Nc3ncc(C(F)(F)F)c(NC4CCCC4)n3)cccc21]

### Step 1

The title compound was made from 2,4-dichloro-5-trifluoromethylpyrimidine following the procedure as described in Example 1, Step 1, except that cyclopentylamine was used in place of methylamine to provide a mixture of isomers containing 2-chloro-N-cyclopentyl-5-(trifluoromethyl)pyrimidin-4-amine (Intermediate 95-a) as a white powder (0.90 g, 75%). LCMS (ESI) m/z 266.1 [M+H]⁺.

### Step 2

The title compound was made from 2-chloro-N-cyclopentyl-5-(trifluoromethyl)pyrimidin-4-amine (Intermediate 95-a) following the procedure as described in Example 1, Step 2, except that 2-(4-amino-1H-indazol-1-yl)acetonitrile (Intermediate 30-b) was used in place of 1H-indazol-6-amine. The crude product was purified by flash chromatography on silica gel column using 0-90% EtOAc/Hexane as eluent, to provide the title compound as a white powder (25.0 mg, 17%). ¹H NMR (400 MHz, DMSO-d₆) δ ppm 1.68-1.54 (br m, 8H), 1.92 (s, 3H), 4.49 (q, J=7.4 Hz, 1H), 577 (s, 2H), 6.62 (d, J=73 Hz, 1H), 7.44-7.40 (m, 2H), 7.89 (dd, J=7.0, 1.4 Hz, 1H), 8.24 (d, J=0.9 Hz, 1H), 8.52 (d, J=0.8 Hz, 1H), 9.89 (s, 1H); ¹⁹F NMR (376 MHz, DMSO-d₆) δ ppm −60.01 (s, 3F); LCMS (ESI) m/z 402.1 [M+H]⁺.

### Example 96

### Synthesis of 2-(4-((4-((3,3-difluorocyclobutyl)amino)-5-(trifluoromethyl)pyrimidin-2-yl)amino)-1H-indazol-1-yl)acetonitrile

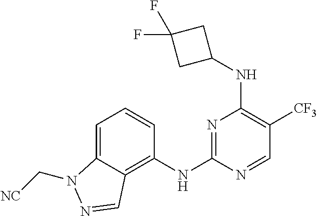 [SMILES: N#CCn1ncc2c(Nc3ncc(C(F)(F)F)c(NC4CC(F)(F)C4)n3)cccc21]

### Step 1

The title compound was made from 2,4-dichloro-5-trifluoromethylpyrimidine following the procedure as described in Example 1, Step 1, except that 3,3-difluorocyclobutanamine hydrochloride was used in place of methylamine to provide a mixture of isomers containing 2-chloro-N-(3,3-difluorocyclobutyl)-5-(trifluoromethyl)pyrimidin-4-amine (intermediate 96-a) as a white powder (0.30 g, 46%). LCMS (ESI) m/z 288.0 [M+H]⁺.

### Step 2

The title compound was made from 2-chloro-N-(3,3-difluorocyclobutyl)-5-(trifluoromethyl)pyrimidin-4-amine (Intermediate 96-a) following the procedure as described in Example 1, Step 2, except that 2-(4-amino-1H-indazol-1-yl)acetonitrile (Intermediate 30-b) was used in place of 1H-indazol-6-amine. The crude product was purified by flash chromatography on silica gel column using 0-90% EtOAc/Hexane as eluent, to provide the title compound as a white powder (25.0 mg, 17%). ¹H NMR (400 MHz, DMSO-4) δ ppm 2.90-2.80 (m, 4H), 4.51-4.44 (m, 1H), 5.77 (s, 2H) 7.44-7.42 (m, 3H), 7.81-7.78 (m, 1H), 8.30 (s, 1H), 8.48 (s, 1H), 9.96 (s, 1H); ¹⁹F NMR (376 MHz, DMSO-d₆) δ ppm −59.96 (s, 5F); LCMS (ESI) m/z 424.1 [M+H]⁺.

### Example 97

### Synthesis of 2-methyl-1-(4-((4-(methylamino)-5-(trifluoromethyl)pyrimidin-2-yl)amino)-1H-indazol-1-yl)propan-2-ol

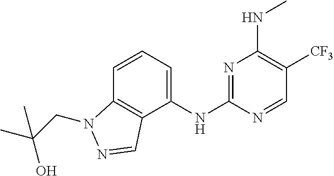 [SMILES: CNc1nc(Nc2cccc3c2cnn3CC(C)(C)O)ncc1C(F)(F)F]

### Step 1

The title compound was made from 4-nitro-1H-indazole following the procedure as described in Example 31, Step 1, except that 1-bromo-2-methylpropan-2-ol was used in place of 2-bromo-2-methylpropionamide. The crude product was purified by flash chromatography on silica gel column using 0-10% EtOAc/CH₂Cl₂ as eluent, to provide 2-methyl-1-(4-nitro-1H-indazol-1-yl)propan-2-ol (Intermediate 97-a) as a pale-yellow solid (150.0 mg, 42%). LCMS (ESI) m/z 236.1 [M+H]⁺.

### Step 2

The title compound was made from 2-methyl-1-(4-nitro-1H-indazol-1-yl)propan-2-ol (Intermediate 97-a) following the procedure as described in Example 33, Step 2. The volatiles were removed under reduced pressure to provide 1-(4-amino-1H-indazol-1-yl)-2-methylpropan-2-ol (Intermediate 97-b) as a pale-yellow solid (26.0 mg, crude). LCMS (ESI) m/z 206.2 [M+H]⁺.

### Step 3

The title compound was made from 2-chloro-N-methyl-5-(trifluoromethyl)pyrimidin-4-amine (intermediate 1-a) following the procedure as described in Example 1, Step 2, except that 1-(4-amino-1H-indazol-1-yl)-2-methylpropan-2-ol (Intermediate 97-b) was used in place of 1H-indazol-6-amine. The crude product was purified by reverse phase preparative HPLC (Gemini® 5 um NX-C18 110 Å, 100×30 mm; mobile phase: [water (10 mM Ammonium Formate pH=3.8 v/v)-MeOH]; B %: 45%-100%, 16 min), to provide the title compound as a white solid (21.0 mg, 44%). ¹H NMR (400 MHz, DMSO-d₆) δ ppm 1.11 (s, 6H), 2.92 (d, J/4.4 Hz, 3H), 4.26 (s, 2H), 4.64 (s, 1H), 7.16 (d, J=4.9 Hz, 1H), 7.33-7.27 (m, 2H), 7.85 (dd, J=6.7, 1.6 Hz, 1H), 8.21 (s, 1H), 8.39 (s, 1H), 9.74 (s, 1H); ¹⁹F NMR (376 MHz, DMSO-d₆) δ ppm −60.4 (s, 3F); LCMS (ESI) m/z 381.3 [M+H]⁺.

### Example 98

### Synthesis of N2-(1-ethyl-1H-indazol-4-yl)-N4-methyl-5-(trifluoromethyl)pyrimidine-2,4 diamine

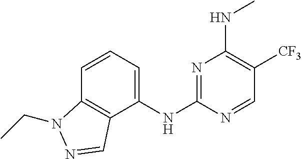 [SMILES: CCn1ncc2c(Nc3ncc(C(F)(F)F)c(NC)n3)cccc21]

### Step 1

The title compounds were made from 4-nitro-1H-indazole following the procedure as described in Example 10, Step 1, except that iodoethane was used in place of iodomethane. The crude products (60:40 ratio of separable N1:N2 regioisomers) were purified by flash chromatography on silica gel column using 50-100% CH₂C₂/Hexanes to provide 1-ethyl-4-nitro-1H-indazole (Intermediate 98-a) (563.0 mg, 48%) and 2-ethyl-4-nitro-2H-indazole (Intermediate 98-b) (435.0 mg, 37%) as yellow solids. LCMS (ESI) m/z 192.1 [M+H]⁺.

### Step 2

The title compound was made from 1-ethyl-4-nitro-1H-indazole (Intermediate 98-a) following the procedure as described in Example 33, Step 2. The volatiles were removed under reduced pressure to provide 1-ethyl-1H-indazol-4-amine (Intermediate 98-c) as a pale-yellow solid (185.0 mg, crude). LCMS (ESI) m/z 162.1 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 1-ethyl-1H-indazol-4-amine (Intermediate 98-c) was used in place of 1H-indazol-6-amine. The crude product was purified by flash chromatography on silica gel column using 0-50% EtOAc/Hexanes as eluent, to provide the title compound as a white solid (50.0 mg, 24%). ¹H NMR (400 MHz, DMSO-d₆) δ ppm 1.38 (t, J=7.2 Hz, 3H), 2.94 (d, J=4.4 Hz, 3H), 4.42 (q, J=7.2 Hz, 2H), 7.17 (s, 1H), 7.33-7.25 (m, 2H), 7.91 (d, J=7.3 Hz, 1H), 8.22 (d, J=1.0 Hz, 1H), 8.39 (d, J=0.9 Hz, 1H), 9.75 (s, 1H); ¹⁹F NMR (376 MHz, DMSO-d₆) δ ppm −60.4 (s, 3F), LCMS (ESI) m/z 337.2 [M+H]⁺.

### Example 99

### Synthesis of N4-cyclopropyl-N2-(1-ethyl-1H-indazol-4-yl)-5-(trifluoromethyl)pyrimidine-2,4-diamine

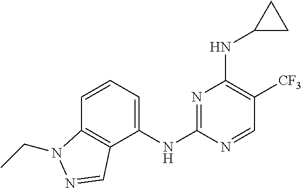 [SMILES: CCn1ncc2c(Nc3ncc(C(F)(F)F)c(NC4CC4)n3)cccc21]

The title compound was made from 2-chloro-N-cyclopropyl-5-(trifluoromethyl)pyrimidin-4-amine (Intermediate 68-a) following the procedure as described in Example 1, Step 2, except that 1-ethyl-1H-indazol-4-amine (Intermediate 98-c) was used in place of 1H-indazol-6-amine. The crude product was purified by flash chromatography on silica gel column using 0-50% EtOAc/Hexanes as eluent, to provide the title compound as an off-white solid (50.0 mg, 33%). ¹H NMR (400 MHz, DMSO-d₆) δ ppm 0.70-0.66 (m, 2H), 0.81-0.76 (m, 2H), 1.37 (t, J=7.2 Hz, 3H), 2.87 (dd, J=7.3, 3.9 Hz, 1H), 4.39 (q, J=7.2 Hz, 2H), 7.21 (s, 1H), 7.32-7.22 (m, 2H), 8.20 (d, J=7.5 Hz, 1H), 8.25 (d, J=0.9 Hz, 1H), 8.46 d, J=0.9 Hz, 1H), 9.84 (s, 1H); ¹⁹F NMR (376 MHz, DMSO-d₆) δ ppm −60.0 (s, 3F); LCMS (ESI) m/z 363.1 [M+H]⁺.

### Example 100

### Synthesis of 2-(7-(1-methyl-1,2,3,6-tetrahydropyridin-4-yl)-4-((4-(methylamino)-5-(trifluoromethyl)pyrimidin-2-yl)amino)-1H-indazol-1-yl)acetamide

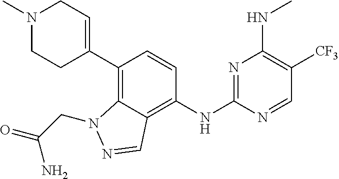 [SMILES: CNc1nc(Nc2ccc(C3=CCN(C)CC3)c3c2cnn3CC(N)=O)ncc1C(F)(F)F]

To a stirring solution of 2-(7-(1-methyl-1,2,3,6-tetrahydropyridin-4-yl)-4-((4-(methylamino)-5-(trifluoromethyl)pyrimidin-2-yl)amino)-1H-indazol-1-yl)acetonitrile (Example 87) (46.8 mg, 106 μmol, 1.00 eq) in a mixture of EtOH (1.50 mL) and water (0.60 mL), was added Ghaffar-Parkins' Catalyst (4.54 mg, 10.6 μmol, 0.10 eq). The reaction mixture was warmed to 80° C. and stirred for 1 h. The reaction mixture was cooled to room temperature, filtered over Celite, and the volatiles were removed under reduced pressure. The crude product was purified by reverse phase chromatography on a C18 column using 3-12% MeCN/H₂O (0.1% FA) as eluent, to provide the title compound as a pale-yellow solid (36.0 mg, 740/0). ¹H NMR (400 MHz, DMSO-d₆) δ ppm 2.31 (s, 3H), 2.37 (s, 2H), 2.61 (t, J=5.5 Hz, 2H), 2.94 (d, J=4.3 Hz, 1H), 3.03 (s, 2H), 4.99 (s, 2H), 5.61-5.63 (m, 1H), 6.99 (d, J=7.9 Hz, 1H), 7.16 (s, 2H), 7.39 (s, 1H), 7.85 (d, J=7.9 Hz, 1H), 8.21 (s, 1H), 8.44 (s, 1H), 9.75 (s, 1H); ¹⁹F NMR (376 MHz, DMSO-d₆) δ ppm −60.4 (s, 3F); LCMS (ESI) m/z 461.2 [M+H]⁺.

### Example 101

### Synthesis of N4-cyclopropyl-N2-(1-(methylsulfonyl)-1H-indazol-4-yl)-5-(trifluoromethyl)pyrimidine-2,4-diamine

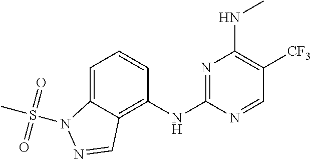 [SMILES: CNc1nc(Nc2cccc3c2cnn3S(C)(=O)=O)ncc1C(F)(F)F]

### Step 1

The title compound was made from 4-nitro-1H-indazole following the procedure as described in Example 33, Step 1. The crude product was triturated with CH₂Cl₂, to provide 1-(methylsulfonyl)-4-nitro-1H-indazole (Intermediate 101-a) as a tan solid (0.85 g, 59%). LCMS (−ESI) m/z 240.0 [M−H]⁻.

### Step 2

The title compound was made from 1-(methylsulfonyl)-4-nitro-1H-indazole (Intermediate 101-a) following the procedure as described in Example 7, Step 1. The volatiles were removed under reduced pressure to provide 1-(methylsulfonyl)-1H-indazol-4-amine (Intermediate 101-b) as a brown oil (0.31 g, crude). LCMS (ESI) m/z 212.0 [M+H]⁺.

### Step 3

The title compound was made from 2-chloro-N-methyl-5-(trifluoromethyl)pyrimidin-4-amine (Intermediate 1-a) following the procedure as described in Example 1, Step 2, except that 1-(methylsulfonyl)-1H-indazol-4-amine (Intermediate 101-b) was used in place of 1H-indazol-6-amine. The crude product was purified by flash chromatography on silica gel column using 0-40% EtOAc/Hexanes as eluent, to provide the title compound as a white solid (92.0 mg, 84%). ¹H NMR (400 MHz, DMSO-d₆) δ ppm 2.91 (d, J=4.4 Hz, 3H), 3.43 (s, 3H), 7.25-7.22 (m, 1H), 7.60-7.53 (m, 2H), 8.10 (d, J=7.3 Hz, 1H), 8.24 (s, 1H), 8.89 (d, J=0.8 Hz, 1H), 10.06 (s, 1H); ¹⁹F NMR (376 MHz, DMSO-d₆) δ ppm −60.6 (s, 3F); LCMS (ESI) m/z 387.0 [M+H]⁺.

### Example 102

### Synthesis of N4-cyclopropyl-N2-(1-(methylsulfonyl)-1H-indazol-4-yl)-5-(trifluoromethyl)pyrimidine-2,4-diamine

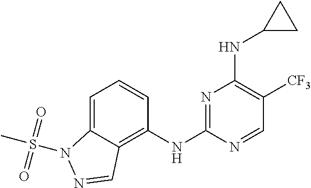 [SMILES: CS(=O)(=O)n1ncc2c(Nc3ncc(C(F)(F)F)c(NC4CC4)n3)cccc21]

The title compound was made from 2-chloro-N-cyclopropyl-5-(trifluoromethyl)pyrimidin-4-amine (Intermediate 68-a) following the procedure as described in Example 1, Step 2, except that 1-(methylsulfonyl)-1H-imidazol-4-amine (Intermediate 101-b) was used in place of 1H-indazol-6-amine. The crude product was purified by reverse phase chromatography on a C18 column using 5-60% MeCN/H₂O (0.1% FA) as eluent, to provide the title compound as a white solid (36.0 mg, 60%). ¹H NMR (400 MHz, DMSO-d₆) δ ppm 0.70-0.66 (m, 2H), 0.79-0.74 (m, 2H), 2.88-2.82 (m, 1H), 3.43 (s, 3H), 7.29-7.28 (m, 1H), 7.59-7.54 (m, 2H), 8.28 (s, 1H), 8.43-8.38 (m, 1H), 8.97 (s, 1H), 10.15 (s, 1H); ¹⁹F NMR (376 MHz, DMSO-d₆) δ ppm −60.2 (s, 3F); LCMS (ESI) m/z 413.2 [M+H]⁺.

### Example 103

### Synthesis of 2-(4-((4-(cyclopropylamino)-5-(trifluoromethyl)pyrimidin-2-yl)amino)-1H-indazol-1-yl)-2-methylpropanenitrile

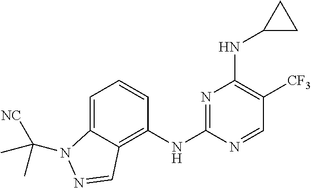 [SMILES: CC(C)(C#N)n1ncc2c(Nc3ncc(C(F)(F)F)c(NC4CC4)n3)cccc21]

The title compound was made from 2-chloro-N-cyclopropyl-5-(trifluoromethyl)pyrimidin-4-amine (Intermediate 68-a) following the procedure as described in Example 1, Step 2, except that 2-(4-amino-1H-indazol-1-yl)-2-methylpropanenitrile (Intermediate 31-d) was used in place of 1H-indazol-6-amine. The crude product was purified by reverse phase chromatography on a C18 column using 5-60% MeCN/H₂O (0.1% FA) as eluent, to provide the title compound as a white solid (46.0 mg, 76%). ¹H NMR (400 MHz, DMSO-d₆) δ ppm 0.70-0.67 (m, 2H), 0.81-0.76 (m, 2H), 2.08 (s, 6H), 2.90-2.84 (m, 1H), 7.25 (s, 1H), 7.48-7.42 (m, 2H), 8.27 (s, 1H), 8.32 (d, J=6.6 Hz, 1H), 8.62 (s, 1H), 9.97 (s, 1H); ¹⁹F NMR (376 MHz, DMSO-d₆) δ ppm −60.1 (s, 3F); LCMS (ESI) m/z 102.2 [M+H]⁺.

### Example 104

### Synthesis of 2-(4-((4-((cyclopropylmethyl)amino)-5-(trifluoromethyl)pyrimidin-2-yl)amino)-1H-indazol-1-yl)acetonitrile

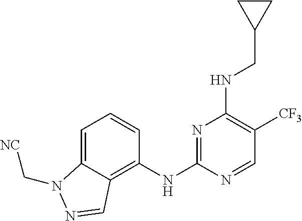 [SMILES: N#CCn1ncc2c(Nc3ncc(C(F)(F)F)c(NCC4CC4)n3)cccc21]

### Step 1

To a stirring solution of 2,4-dichloro-5-(trifluoromethyl)pyrimidine (791 μL, 5.75 mmol, 1.10 eq) in a 1:1 mixture of DCE and t-BuOH (21.0 mL) cooled to 0° C., ZnCl₂ (1 M in THF) (6.53 mL, 6.53 mmol, 1.25 eq) was added dropwise. The reaction mixture was stirred at 0° C. under argon atmosphere for 1 h. Then 2-(4-amino-1H-indazol-1-yl)acetonitrile (Intermediate 30-b) (900 mg, 5.23 mmol, 1.00 eq) and NEt₃ (805 μL, 5.75 mmol, 1.10 eq) were added and the reaction mixture was warmed to room temperature and stirred for 48 h. The volatiles were removed under reduced pressure and the crude residue was dissolved in CH₂Cl₂ (10 mL) and washed with water (15 mL).

The organic layer was dried over MgSO₄, filtered, and the volatiles removed under reduced pressure. The crude product was triturated with CH₂Cl₂, to provide 2-(4-((4-chloro-5-(trifluoromethyl)pyrimidin-2-yl)amino)-1H-indazol-1-yl)acetonitrile (Intermediate 104-a) as a tan powder (620 mg, 32% yield). LCMS (ESI) m/z 353.1 [M+H]⁺.

### Step 2

To a stirring solution of 2-(4-((4-chloro-5-(trifluoromethyl)pyrimidin-2-yl)amino)-1H-indazol-1-yl)acetonitrile (Intermediate 104-a) (60.0 mg, 170 μmol, 1.00 eq) in dioxane (1.70 mL), was added DIPEA (90.7 μL, 510 μmol, 3.00 eq) and cyclopropanemethylamine (60.5 mg, 851 μmol, 5.00 eq). The reaction mixture was warmed to 80° C. and stirred for 30 min. The reaction mixture was cooled to room temperature and the volatiles were removed under reduced pressure. The crude product was purified by flash chromatography on silica gel column using 0-45% EtOAc/Hexanes as eluent, to provide the title compound as a white solid (50.8 mg, 77%). ¹H NMR (400 MHz, DMSO-d₆) δ ppm 0.24-0.20 (m, 2H), 0.40-0.35 (m, 2H), 1.18-1.11 (m, 1H), 3.33-3.30 (m, 2H), 5.77 (s, 2H), 7.25 (t, J=5.6 Hz, 1H), 7.44-7.38 (m, 2H), 7.92 (d, J=7.0 Hz, 1H), 8.24 (d, J=1.0 Hz, 1H), 8.53-8.53 (m, 1H), 9.87 (s, 1H); ¹⁹F NMR (376 MHz, DMSO-d₆) δ ppm −60.4 (s, 3F); LCMS (ESI) m/z 388.1 [M+H]⁺.

### Example 105

### Synthesis of 2-(4-((4-((1-methylcyclopropyl)amino)-5-(trifluoromethyl)pyrimidin-2-yl)amino)-1H-indazol-1-yl)acetonitrile

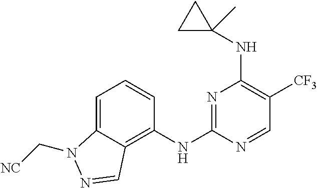 [SMILES: CC1(Nc2nc(Nc3cccc4c3cnn4CC#N)ncc2C(F)(F)F)CC1]

The title compound was made from 2-(4-((4-chloro-5-(trifluoromethyl)pyrimidin-2-yl)amino)-1H-indazol-1-yl)acetonitrile (Intermediate 104-a) following the procedure as described in Example 104, Step 2, except that 1-methylcyclopropanamine was used in place of cyclopropanemethylamine. The crude product was purified by flash chromatography on silica gel column using 0-90% EtOAc/Hexane as eluent, to provide the title compound as a white powder (30.0 mg, 39%). ¹H NMR (400 MHz, DMSO-d₆) δ ppm 0.75-0.74 (m, 2H), 0.83-0.82 (m, 2H), 1.41 (s, 3H), 5.77 (s, 2H), 7.48-7.36 (m, 3H), 8.26 (s, 1H), 8.45 (d, J=77 Hz, 1H), 8.67 (d, J=0.9 Hz, 1H, 9.96 (s, 1H); ¹⁹F NMR (376 MHz, DMSO-d₆) δ ppm −60.0 (s, 3F); LCMS (ESI) m/z 388.1 [M+H]⁺.

### Example 106

### Synthesis of 2-(4-((4-((2,2,2-trifluoroethyl)amino)-5-(trifluoromethyl)pyrimidin-2-yl)amino)-1-indazol-1-yl)acetonitrile

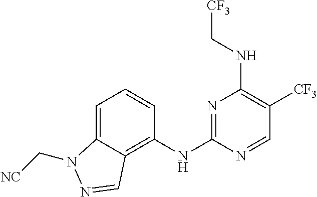 [SMILES: N#CCn1ncc2c(Nc3ncc(C(F)(F)F)c(NCC(F)(F)F)n3)cccc21]

The title compound was made from 2-(4-((4-chloro-5-(trifluoromethyl)pyrimidin-2-yl)amino)-1H-indazol-1-yl)acetonitrile (Intermediate 104-a) following the procedure as described in Example 104, Step 2, except that 2,2,2-trifluoroethylamine was used in place of cyclopropanemethylamine. The crude product was purified by flash chromatography on silica gel column using 0-90% EtOAc/Hexane as eluent, to provide the title compound as a white powder (45.0 mg, 55%). ¹H NMR (400 MHz, DMSO-d₆) δ ppm 4.28-4.22 (m, 2H), 5.78 (s, 2H), 7.46-7.40 (m, 2H, 7.65-7.62 (m, 1H), 7.79 (dd, J=6.7, 1.7 Hz, 1H), 8.38 (d, J=0.9 Hz, 1H), 8.49 (d, J=0.8 Hz, 1H), 10.09 (s, 1H); ¹⁹F NMR (376 MHz, DMSO-d₆) δ ppm −60.0 (s, 3F), −70.0 (s, 3F); LCMS (ESI) m/z 416.1 [M+H]⁺.

### Example 107

### Synthesis of 2-(4-((4-(dimethylamino)-5-(trifluoromethyl)pyrimidin-2-yl)amino)-1H-indazol-1-yl)acetonitrile

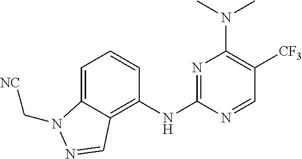 [SMILES: CN(C)c1nc(Nc2cccc3c2cnn3CC#N)ncc1C(F)(F)F]

The title compound was made from 2-(4-((4-chloro-5-(trifluoromethyl)pyrimidin-2-yl)amino)-1H-indazol-1-yl)acetonitrile (Intermediate 104-a) following the procedure as described in Example 104, Step 2, except that dimethylamine was used in place of cyclopropanemethylamine. The crude product was purified by flash chromatography on silica gel column using 0-45% EtOAc/Hexanes as eluent, to provide the title compound as a white solid (47.1 mg, 77%). ¹H NMR (400 MHz, DMSO-d₆) δ ppm 3.12 (s, 6H), 5.77 (s, 2H), 7.47-7.39 (m, 2H), 7.89 (d, J=7.4 Hz, 1H), 8.42 (s, 1H), 8.53-8.53 (m, 1H), 9.96 (s, 1H); ¹⁹F NMR (376 MHz, DMSO-d₆) δ ppm −51.7 (s, 3F); LCMS (ESI) m/z 362.1 [M+H]⁺.

### Example 108

### Synthesis of 2-(4-((4-(isopropylamino)-5-(trifluoromethyl)pyrimidin-2-yl)amino)-1H-indazol-1-yl)-2-methylpropanenitrile

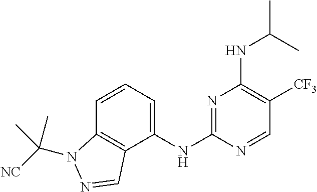 [SMILES: CC(C)Nc1nc(Nc2cccc3c2cnn3C(C)(C)C#N)ncc1C(F)(F)F]

### Step 1

The title compound was made from 2,4-dichloro-5-(trifluoromethyl)pyrimidine following the procedure as described in Example 104, Step 1, except that 2-(4-amino-1H-indazol-1-yl)-2-methylpropanenitrile (Intermediate 31-d) was used in place of 2-(4-amino-1H-indazol-1-yl)acetonitrile (Intermediate 30-b). The crude product was purified by flash chromatography on silica gel column using 0-30% EtOAc/Hexanes as eluent, to provide 2-(4-(4-chloro-5-(trifluoromethyl)pyrimidin-2-yl)amino)-1H-indazol-yl)-2-methylpropanenitrile (Intermediate 108-a) as a golden-yellow solid (347.0 mg, 61%). LCMS (ESI) m/Z 381.2 [M+H]⁺.

### Step 2

The title compound was made from 2-(4-((4-chloro-5-(trifluoromethyl)pyrimidin-2-yl)amino)-1H-indazol-1-yl)-2-methylpropanenitrile (Intermediate 108-a) following the procedure as described in Example 104, Step 2, except that isopropylamine was used in place of cyclopropanemethylamine. The crude product was purified by reverse phase preparative HPLC (Gemini® 5 um NX-C18 110 Å, 100×30 mm; mobile phase: [water (10 mM Ammonium Formate pH=3.8 v/v)-MeCN]; B %: 50%-100%, 16 min), to provide title compound as a white solid (27.0 mg, 43%). ¹H NMR (400 MHz, DMSO-d₆) δ ppm 1.22 (d, J=6.6 Hz, 6H), 2.09 (s, 6H), 4.50-4.45 (m, 1H), 6.58 (d, J=8.0 Hz, 1H), 7.50-7.46 (m, 2H), 7.92 (d, J=7.4 Hz, 1H), 8.24 (d, J=1.0 Hz, 1H), 8.53 (d, J=0.9 Hz, 1H), 9.87 (s, 1H); ¹⁹F NMR (376 MHz, DMSO-d₆) δ ppm −60.1 (s, 3F); LCMS (ESI) m/z 404.3 [M+H]⁺.

### Example 109

### Synthesis of 2-(4-((4-(methylamino)-5-(trifluoromethyl)pyrimidin-2-yl)amino)-7-(1-methylpiperidin-4-yl)-1H-indazol-1-yl)acetonitrile

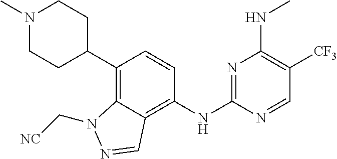 [SMILES: CNc1nc(Nc2ccc(C3CCN(C)CC3)c3c2cnn3CC#N)ncc1C(F)(F)F]

### Step 1

The title compound was made from 2-(7-(1-methyl-1,2,3,6-tetrahydropyridin-4-yl)-4-((4-(methylamino)-5-(trifluoromethyl)pyrimidin-2-yl)amino)-1H-indazol-1-yl)acetamide (Example 100) following the procedure as described in Example 82, Step 1, except that a 4:1 mixture of EtOH and NMP was used and the reaction mixture was stirred for 2 days. The crude product was purified by reverse phase chromatography on a C18 column using 5-15% MeCN/H₂O (0.1% FA) as eluent, to provide 2-(4-((4-(methylamino)-5-(trifluoromethyl)pyrimidin-2-yl)amino)-7-(1-methylpiperidin-4-yl)-1H-indazol-1-yl)acetamide-1 (Intermediate 109-a) as a white powder (12.1 mg, 43%). LCMS (ESI) m/z 463.4 [M+H]⁺.

### Step 2

A stirring solution of 2-(4-((4-(methylamino)-5-(trifluoromethyl)pyrimidin-2-yl)amino)-7-(1-methylpiperidin-4-yl)-1H-indazol-1-yl)acetamide-1 (Intermediate 109-a) (9.00 mg, 19.5 μmol, 1.00 eq) dissolved in POCl₃ (163 uL, 1.75 mmol, 90.0 eq) was warmed to 90° C. and stirred for 30 min. The reaction mixture was cooled to 0° C., diluted with CH₂Cl₂, and neutralized with a saturated solution of NaHCO₃. The layers were separated, and the aqueous layer was extracted with CH₂Cl₂ (10 mL×3). The combined organic layers were washed with brine, dried over MgSO₄, filtered, and concentrated under reduced pressure. The crude product was purified by reverse phase preparative HPLC (Gemini® 5 um NX-C18 110 Å, 100×30 mm; mobile phase: [water (10 mM Ammonium Bicarbonate pH=10.0 v/v)-MeCN]; B %: 55%-100%, 16 min) to provide the title compound as a white powder (4.3 mg, 50%). ¹H NMR (400 MHz, DMSO-d₆) δ ppm 1.87-1.76 (m, 4H), 2.18-2.12 (m, 2H), 2.23 (s, 3H), 2.93-2.91 (m, 5H), 3.13-3.07 (m, 1H), 5.77 (s, 2H), 7.18-7.15 (m, 1H), 7.31 (d, J=8.1 Hz, 1H), 7.89 (d, J=8.0 Hz, 1H), 8.20 (s, 1H), 8.52 (s, 1H), 9.75 (s, 1H); ¹⁹F NMR (376 MHz, DMSO-d₆) δ ppm −60.4 (s, 3F); LCMS (ESI) m/z 445.2 [M+H]⁺.

### Example 110

### Synthesis of 2-(4-((4-(isopropylamino)-5-(trifluoromethyl)pyrimidin-2-yl)amino)-1H-indazol-1-yl)acetonitrile

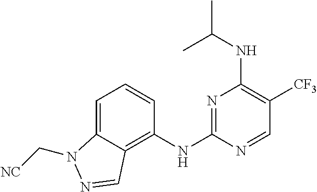 [SMILES: CC(C)Nc1nc(Nc2cccc3c2cnn3CC#N)ncc1C(F)(F)F]

The title compound was made from 2-(4-((4-chloro-5-(trifluoromethyl)pyrimidin-2-yl)amino)-1H-indazol-1-yl)acetonitrile (Intermediate 104-a) following the procedure as described in Example 104, Step 2, except that isopropylamine was used in place of cyclopropanemethylamine. The crude product was purified by flash chromatography on silica gel column using 0-90% EtOAc/Hexane as eluent, to provide the title compound as a white powder (20.0 mg, 13%). ¹H NMR (400 MHz, DMSO-d₆) δ ppm 1.22 (d, J=6.6 Hz, 6H), 4.49-4.44 (m, 1H), 6.57 (d, J=8.0 Hz, 1H), 7.45-7.37 (m, 2H), 7.89 (dd, J=7.3, 1.1 Hz, 1H), 8.24 (d, J=1.0 Hz, 1H), 8.53 (d, J=0.9 Hz, 1H), 9.87 (s, 1H); ¹⁹F NMR (376 MHz, DMSO-d₆) δ ppm −60.0 (s, 3F); LCMS (ESI) m/z 376.1 [M+H]⁺.

### Example 111

### Synthesis of 2-(7-chloro-4-((4-(methylamino)-5-(trifluoromethyl)pyrimidin-2-yl)amino)-1H-indazol-1-yl)acetonitrile

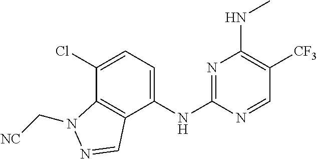 [SMILES: CNc1nc(Nc2ccc(Cl)c3c2cnn3CC#N)ncc1C(F)(F)F]

### Step 1

The title compound was made from 4-bromo-7-chloro-1H-indazole following the procedure as described in Example 16, step 1. The crude product was purified by flash chromatography on silica gel column using 0-40% EtOAc/Hexanes as eluent, to provide 2-(4-bromo-7-chloro-1-1-indazol-1-yl)acetonitrile (Intermediate 111-a) as a white solid (205.0 mg, 70%). ¹H NMR (400 MHz, DMSO-d₆) δ ppm 5.99 (s, 2H), 7.48 (d, J=8.0 Hz, 1H), 7.54 (d, J=8.0 Hz, 1H), 8.33 (s, 1H).

### Step 2

The title compound was made from 2-(4-bromo-7-chloro-1H-indazol-1-yl)acetonitrile (Intermediate 111-a) following the procedure as described in Example 4, Step 3, except that 2-(4-bromo-7-chloro-1H-indazol-1-yl)acetonitrile (Intermediate 111-a) was used in place of 6-bromo-7-methoxy-1-(tetrahydro-2H-pyran-2-yl)-1H-indazole (Intermediate 4-b). The crude product was purified by flash chromatography on silica gel column using 0-30% EtOAc/Hexanes as eluent, to provide tert-butyl (7-chloro-1-(cyanomethyl)-1H-indazol-4-yl)carbamate (Intermediate 111-b) as a pale-yellow solid (147.0 mg, 86%). LCMS (ESI) m/z 307.1 [M+H]⁺.

### Step 3

The title compound was made from tert-butyl (7-chloro-1-(cyanomethyl)-1H-indazol-4-yl)carbamate (Intermediate 111-b) following the procedure as described in Example 4, Step 4. The volatiles were removed under reduced pressure to provide 2-(4-amino-7-chloro-1H-indazol-1-yl)acetonitrile (Intermediate 111-c) as a brown oil (100 mg, crude). LCMS (ESI) m/z 207.1 [M+H]⁺.

### Step 4

The title compound was made from 2-chloro-N-methyl-5-(trifluoromethyl)pyrimidin-4-amine (intermediate 1-a) following the procedure as described in Example 1, Step 2, except that 2-(4-amino-7-chloro-1H-indazol-1-yl)acetonitrile (Intermediate 111-c) was used in place of 1H-indazol-6-amine. The crude product was purified by flash chromatography on silica gel column using 0-30% EtOAc/Hexanes as eluent, to provide title compound as a white solid (16.0 mg, 8.7%). ¹H NMR (400 MHz, DMSO-d₆) δ ppm 292 (d, J=4.4 Hz, 3H), 5.92 (s, 2H), 7.25 (s, 1H), 7.50 (d, J=8.4 Hz, 1H), 8.01 (d, J=8.4 Hz, 1H), 8.24 (s, 1H), 8.67 (s, 1H), 9.99 (s, 1H); ¹⁹F NMR (376 MHz, DMSO-d₆) δ ppm −60.6 (s, 3F); LCMS (ESI) m/z 382.1 [M+H]⁺.

### Example 112

### Synthesis of 2-methyl-2-(4-((4-((2,2,2-trifluoroethyl)amino)-5-(trifluoromethyl)pyrimidin-2-yl)amino)-1H-indazol-1-yl)propanenitrile

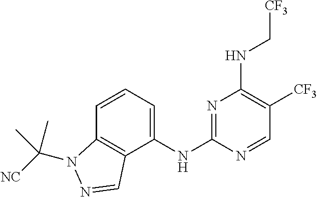 [SMILES: CC(C)(C#N)n1ncc2c(Nc3ncc(C(F)(F)F)c(NCC(F)(F)F)n3)cccc21]

The title compound was made from 2-(4-((4-chloro-5-(trifluoromethyl)pyrimidin-2-yl)amino)-1H-indazol-1-yl)-2-methylpropanenitrile (Intermediate 108-a) following the procedure as described in Example 104, Step 2, except that 2,2,2-trifluoroethylamine was used in place of cyclopropanemethylamine. The crude product was purified by flash chromatography on silica gel column using 0-30% EtOAc/Hexanes as eluent, to provide title compound as a white solid (52.5 mg, 75%). ¹H NMR (400 MHz, DMSO-d₆) δ ppm 2.16 (s, 6H), 4.33 (s, 3H), 7.49 (t, J=8.1 Hz, 1H), 7.60 (d, J=8.5 Hz, 1H), 7.70 (s, 1H), 7.88 (d, J=7.7 Hz, 1H), 8.45 (s, 1H), 8.55 (d, J=0.9 Hz, 1H), 10.16 (s, 1H); ¹H NMR (376 MHz, DMSO-d₆) δ ppm −70.1 (t, 3F), −601 (s, 3F); LCMS (ESI) m/z 444.2 [M+H]⁺.

### Example 113

### Synthesis of 2-(4-((4-(cyclobutylamino)-5-(trifluoromethyl)pyrimidin-2-yl)amino)-1H-indazol-1-yl)-2-methylpropanenitrile

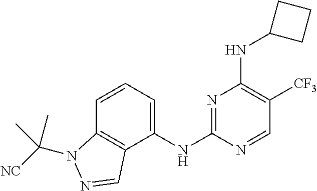 [SMILES: CC(C)(C#N)n1ncc2c(Nc3ncc(C(F)(F)F)c(NC4CCC4)n3)cccc21]

The title compound was made from 2-(4-((4-chloro-5-(trifluoromethyl)pyrimidin-2-yl)amino)-1H-indazol-1-yl)-2-methylpropanenitrile (Intermediate 103-a) following the procedure as described in Example 104, Step 2, except that cyclobutylamine was used in place of cyclopropanemethylamine. The crude product was purified by flash chromatography on silica gel column using 0-30% EtOAc/Hexanes as eluent, to provide title compound as a white solid (51.4 mg, 79%) ¹H NMR (400 MHz, DMSO-d₆) δ ppm 1.69-1.60 (m, 2H), 2.09 (s, 6H), 2.22-2.13 (m, 5H), 4.65-4.59 (m, 1H), 7.08 (d, J=7.0 Hz, 1H), 7.52-7.44 (m, 2H), 7.91 (dd, J=7.3, 1.0 Hz, 1H), 8.25 (d, J=0.9 Hz, 1H), 8.50 (d, J=0.9 Hz 1H), 9.88 (s, 1H); ¹⁹F NMR (376 MHz, DMSO-d₆) δ ppm −60.0 (s, 3F); LCMS (ESI) m/z 416.4 [M+H]⁺.

### Example 114

### Synthesis of N²-(5-fluoro-1H-indazol-6-yl)-N⁴-methyl-5-(trifluoromethyl)pyrimidine-2,4-diamine

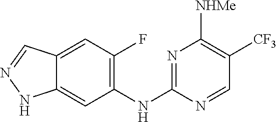 [SMILES: CNc1nc(Nc2cc3[nH]ncc3cc2F)ncc1C(F)(F)F]

### Step 1

To a solution of 5-fluoro-6-nitro-1H-indazole (150 mg, 828 □mol) in EtOH (9 mL) and H₂O (3 mL) were added Fe (462 mg, 8.28 mmol) and NH₄C (443 mg, 8.28 mmol) The mixture was stirred at 50° C. for 5 hrs. The reaction mixture was filtered and concentrated to remove the organic solvent. The residue was extracted with EtOAc (10 mL×2), washed with brine (10 mL) and dried over Na₂SO₄ and concentrated, to provide 5-fluoro-1H-indazol-6-amine (Intermediate 114-a) as a waxy yellow solid (120 mg, 95.9%), which was used in next step without further purification. ¹H NMR (400 MHz, DMSO-d₆) δ ppm 5.31 (s, 2H), 6.72 (d, J=7.5 Hz, 1H), 7.30 (d, J=11.3 Hz, 1H), 7.76 (s, 1H), 12.43 (br s, 1H).

### Step 2

To a solution of 2-chloro-N-methyl-5-(trifluoromethyl)pyrimidin-4-amine (Intermediate 1-a) (168 mg, 0.794 mmol) and 5-fluoro-1H-indazol-6-amine (Intermediate 114-a) (120 mg, 0.794 mmol) in IPA (3 mL) was added 1M HCl (79 μL). The mixture was stirred at 80° C. for 5 hrs. The solution was concentrated to give a crude product which was triturated with EtOAc (10 mL×2) at 25° C. for 15 mins to provide the title compound as a yellow solid (113 mg, 43.6%). ¹H NMR (400 MHz, DMSO-d₆) δ ppm 2.97 (d, J=4.4 Hz, 3H) 7.68 (d, J=10.8 Hz, 1H), 8.06 (d, J=0.6 Hz, 1H), 8.21 (br d, J=5.6 Hz, 1H), 8.36 (br s, 1H), 8.46 (br s, 1H), 10.22 (br s, 1H); LCMS (ESI) m/z 326.9 [M+H]⁺.

### Example 115

### Synthesis of N⁴-methyl-N²-pyrazolo[1,5-a]pyrimidin-7-yl-5-(trifluoromethyl)pyrimidine-2,4-diamine

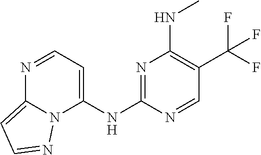 [SMILES: CNc1nc(Nc2ccnc3ccnn23)ncc1C(F)(F)F]

### Step 1

A mixture of 7-chloropyrazolo [1,5-a]pyrimidine (500 mg, 3.26 mmol) in NH₃·H₂O (10 mL) was stirred at 50° C. for 3 hrs. The reaction was concentrated to give crude product pyrazolo [1,5-a]pyrimidin-7-amine (Intermediate 115-a) as white solid, which was used into the next step without further purification. (400 mg, crude). LCMS (ESI) m/z 135.6 [M+H]⁺.

### Step 2

A mixture of pyrazolo[1,5-a]pyrimidin-7-amine (Intermediate 115-a) (200 mg, 1.49 mmol), 2-chloro-N-methyl-5-(trifluoromethyl)pyrimidin-4-amine (intermediate 1-a) (315 mg, 1.49 mmol), HCl (1 M, 149 uL) in i-PrOH (10 mL), then the mixture was stirred at 80° C. for 4 hrs. The reaction was evaporated, and the residue was purified by prep-HPLC (column: YMC C18; mobile phase: [water (HCl)-ACN]; B %: 8%-48%, 9 min) to provide the title compound was obtained as a yellow solid (97 mg, 21.0%). ¹H NMR (400 MHz, DMSO-d₆) δ ppm 3.09 (d, J=4.40 Hz, 3H), 6.82 (d, J=8.07 Hz, 1H), 7.54 (d, J=2.08 Hz, 1H), 8.20 (br d, J=4.40 Hz, 1H), 8.50 (d, J=2.08 Hz, 1H), 8.68 (s, 1H), 9.41 (d, J=8.07 Hz, 1H), 10.64 (s, 1H), 11.15 (br s, 1H); LCMS (ESI) m/z 309.9 [M+H]⁺.

### Example 116

### Synthesis of 3-[[4-(methylamino)-5-(trifluoromethyl)pyrimidin-2-yl]amino]pyrazolo[1,5-a]pyridine-5-carbonitrile

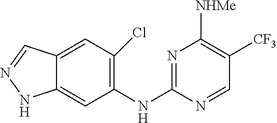 [SMILES: CNc1nc(Nc2cc3[nH]ncc3cc2Cl)ncc1C(F)(F)F]

### Step 1

A mixture of 4-chloro-2-methyl-aniline (4 g, 28.2 mmol) and HNO₃ (3.56 g, 56.5 mmol) in H₂SO₄ (10 mL) was stirred at 0° C. for 2 hrs. The reaction mixture was diluted with the cooled H₂O (50 mL), and then neutralized by NaHCO₃ (50 mL). The precipitate yellow crystals were filtered and dried to provide 4-chloro-2-methyl-5-nitro-aniline (Intermediate 116-a) as a yellow solid (3.0 g, 56.9%). ¹H NMR (400 MHz, DMSO-d₆) δ ppm 2.20 (s, 3-1), 7.24 (d, 2H).

### Step 2

A mixture of 4-chloro-2-methyl-5-nitro-aniline (Intermediate 116-a) (500 mg, 2.68 mmol), acetyl acetate (820 mg, 8.04 mmol), KOAc (315 mg, 3.22 mmol) and isopentyl nitrite (627 mg, 5.36 mmol) in MeCN (20 ml) was degassed and purged with N₂ for 3 times, and then the mixture was stirred at 80° C. for 16 hrs under N₂ atmosphere. Then the solvent was evaporated in vacuum. The residue was purified by flash chromatography (ISCO®; 12 g SepaFlash® Silica Flash Column, petroleum ether/EtOAc with EtOAc from 0˜50%, 20 mL/min, 254 mn/I₂) to provide 5-chloro-6-nitro-1H-indazole (Intermediate 116-b) as a white solid (200 mg, 37.78% yield). ¹H NMR (400 MHz, DMSO-d₆) δ ppm 8.22 (s, 1H), 8.28 (s, 1H), 8.36 (s, 1H), 13.87 (br s, 1H); LCMS (ESI) m/z 197.8 [M+H]⁺.

### Step 3

A mixture of 5-chloro-6-nitro-1H-indazole (Intermediate 116-b) (200 mg, 1.01 mmol), Fe (565 mg, 10.1 mmol) and N₄Cl (541 mg, 10.1 mmol) in EtOH/H₂O (3:1, 20 mL) was stirred at 60° C. for 2 hrs. The reaction mixture was filtered and concentrated. The residue was extracted with EtOAc (20 mL×3) and concentrated under reduced pressure to give product 5-chloro-1H-indazol-6-amine (intermediate 116-c) as brown solid, which was used into the next step without further purification (100 mg, 59.0%). LCMS (ESI) m/z 167.8 [M+H]⁺.

### Step 4

A mixture of 5-chloro-1H-indazol-6-amine (Intermediate 116-c) (100 mg, 596 μmol), 2-chloro-N-methyl-5-(trifluoromethyl) pyrimidin-4-amine (Intermediate 1-a) (126 mg, 596 μmol) and HCl (1 M, 596 μL) in i-PrOH (10 mL), then the mixture was stirred at 80° C. for 2 hrs. i-PrOH was evaporated, the resulting crude product was triturated with MeOH at 25° C. for 60 mins to provide the title compound as a white solid (22 mg, 10.8%). ¹H NMR (400 MHz, DMSO-d₆) δ (ppm 2.90 (br d, J=3.91 Hz, 3H), 790-821 (m, 3H), 8.28 (br s, 1H), 8.41 (br s, 1H), 9.99 (br s, 1H); LCMS (ESI) m/z 342.9 [M+H]⁺.

### Example 117

### Synthesis of N²-(imidazo[1,5-a]pyridin-1-yl)-N4-methyl-5-(trifluoromethyl)pyrimidine-2,4-diamine

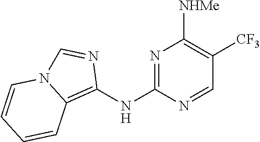 [SMILES: CNc1nc(Nc2ncn3ccccc23)ncc1C(F)(F)F]

### Step 1

A solution of imidazo[1,5-a]pyridine (1 g, 8.46 mmol) and NBS (1.51 g, 8.46 mmol) in DCM (30 mL) was stirred at −78-25° C. for 3 hrs. The reaction mixture was concentrated under reduced pressure to remove solvent and the residue was purified by flash silica gel chromatography (ISCO®; 12 g SepaFlash® Silica Flash Column, Eluent of 0-50% Ethyl acetate/Petroleum ethergradient @16 mL/min), to provide 1-bromoimidazo [1,5-a]pyridine (Intermediate 117-a) as a yellow solid (800 mg, 47.9%). LCMS (ESI) m/z 199.2 [M+H]⁺.

### Step 2

A mixture of 1-bromoimidazo[1,5-a]pyridine (Intermediate 117-a) (730 mg, 3.70 mmol), diphenylmethanimine (873 mg, 4.82 mmol), (1E,4E)-1,5-diphenylpenta-1,4-dien-3-one; palladium (339 mg, 370 □mol), [1-(2-diphenylphosphanyl-1-naphthyl)-2-naphthyl]-diphenyl-phosphane (692 mg, 1.11 mmol) and sodium; 2-methylpropan-2-olate (534 mg, 5.56 mmol) in toluene (25 mL) was stirred at 80° C. for 16 hrs under N₂ atmosphere. The reaction mixture was concentrated under reduced pressure and the residue was purified by flash silica gel chromatography (ISCO®; 12 g SepaFlash® Silica Flash Column, Eluent of 0˜100% Ethyl acetate/Petroleum ethergradient @12 mL/min), to provide N-imidazo [1,5-a] pyridin-1-yl-1,1-diphenyl-methanimine (Intermediate 117-b) as a yellow oil (800 mg, 72.6% yield). ¹H NMR (400 MHz, CDCl₃) 6.49-6.56 (m, 1H), 6.64-6.72 (m, 1H), 7.28-7.33 (m, 2H), 7.35-7.46 (m, 7H), 7.63-7.69 (m, 1H), 7.70-7.75 (m, 1H), 7.76-7.79 (m, 1H), 7.79-7.86 (n, 2H).

### Step 3

A mixture of N-imidazo[1,5-a]pyridin-1-yl-1,1-diphenyl-methanimine (Intermediate 117-b) (500 mg, 1.68 mmol) in HCl/MeOH (4 M, 6 mL) was stirred at 25° C. for 1. The reaction mixture was concentrated under reduced pressure to remove solvent. The residue will be used for next step without purification. Compound imidazo [1,5-a]pyridin-1-amine (Intermediate 117-c) (12.0 mg, 53.6% yield) as obtained as a yellow solid.

### Step 4

A stirred solution of imidazo [1,5-a]pyridin-1-amine (Intermediate 117-c) (12.0 mg, 901 μmol), 2-chloro-N-methyl-5-(trifluoromethyl)pyrimidin-4-amine (Intermediate 1-a) (190 mg, 901 μmol) in i-PrOH (10 ml) was added with HCl (1 M, 901 μL) was stirred at 80° C. for 30 mins. The reaction mixture was concentrated under reduced pressure and purified by prep-HPLC (YMC-Actus Triart C18 150×30 mm×5 μm; mobile phase: [water (HCl)-ACN]; B %: 5%-45%, 9 mins), to provide the title compound as a white solid (14 mg, 5.04% yield). ¹H NMR (400 MHz, DMSO-d₆) 2.68 (br s, 1H), 2.71-3.17 (m, 3H), 4.74 (br d, J=13.05 Hz, 12H), 6.67-6.99 (m, 2H), 7.47-8.04 (m, 1H), 8.18-8.75 (m, 4H), 11.45-12.06 (M, 1H); LCMS (ESI) m/z 309.0 [M+H]⁺.

### Example 118

### Synthesis of 3-[[4-(methylamino)-5-(trifluoromethyl)pyrimidin-2-yl]amino]pyrazolo[1,5-a]pyridine-5-carbonitrile

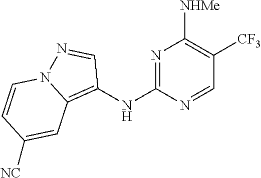 [SMILES: CNc1nc(Nc2cnn3ccc(C#N)cc23)ncc1C(F)(F)F]

A mixture of methyl 3-bromopyrazolo[1,5-a]pyridine-5-carboxylate (440 mg, 1.73 mmol), and NaOH (179 mg, 4.49 mmol) in MeOH/H₂O (3:1, 2 mL) was stirred at 25° C. for 3 hrs. The reaction was acidified with 6 N HCl to pH=2 and extracted with ethyl acetate (20 mL×2). The combined organic phase was washed with brine (20 mL), dried with anhydrous Na₂SO₄, filtered and concentrated in vacuum, to provide 3-bromopyrazolo[1,5-a]pyridine-5-carboxylic acid (Intermediate 118-a) (215 mg, 51.9% yield). ¹H NMR (400 MHz, DMSO-d₆) δ ppm 6.01 (dd, J=7.91, 2.32 Hz, 2H), 7.03 (d, J=8.20 Hz, 1H).

### Step 2

A mixture of 3-bromopyrazolo[1,5-a]pyridine-5-carboxylic acid (Intermediate 118-a) (216 mg, 896 μmol), HATU (374 mg, 986 μmol), NH₄Cl (95.8 mg, 1.79 mmol) and DIEA (347 mg, 2.69 mmol) in DMF (1.5 mL) was stirred at 25° C. for 3 hrs. The solution was quenched with 5 mL of water and extracted with ethyl acetate (20 mL×2). The combined organic phase was washed with brine (20 mL), dried with anhydrous Na₂SO₄, filtered and concentrated in vacuum. The residue was purified by flash chromatography (ISCO®; 4 g SepaFlash® Silica Flash Column, petroleum ether/EtOAc with EtOAc from 0˜50%, 100 mL/min, 254 mn) to afford 3-bromopyrazolo [1,5-a]pyridine-5-carboxamide (Intermediate 118-b) as a yellow solid (145 mg, 67.4% yield). ¹H NMR (400 MHz, DMSO-d₆) δ ppm 7.34-7.41 (m, 1H), 7.62-7.69 (m, 1H), 8.15-8.19 (m, 1H), 8.27 (s, 1H), 8.30-8.36 (m, 1H), 8.77-8.86 (m, 1H); LCMS (ESI) m/z 240.1 [M+H]⁺.

### Step 3

A mixture of 3-bromopyrazolo [1,5-a]pyridine-5-carboxamide (Intermediate 118-b) (145 mg, 604 μmol), Et₃N (611 mg, 6.04 mmol) and TFAA (634 mg, 3.02 mmol) in MeCN (5 mL) was stirred at 25° C. for 16 hrs under N₂ atmosphere. The solution was was quenched with and extracted with ethyl acetate (20 mL×2). The combined organic phase was washed with brine (20 mL), dried with anhydrous Na₂SO₄, filtered and concentrated in vacuum. the residue was purified by flash chromatography (ISCO®; 4 g SepaFlash® Silica Flash Column, petroleum ether/EtOAc with EtOAc from 0-50%, 5 mL/min, 254 inn) to afford 3-bromopyrazolo[1,5-a]pyridine-5-carbonitrile (Intermediate 118-c) (134 mg, 99.91% yield) as a white solid. ¹H NMR (400 MHz, DMSO-d₆) δ ppm 7.11-7.44 (m, 1H), 8.31-8.54 (m, 2H), 8.87-9.15 (m, 1H). LCMS (ESI) m/z 222.0 [M+H]⁺.

### Step 4

A mixture of 3-bromopyrazolo[1,5-a]pyridine-5-carbonitrile (Intermediate 118-c) (134 mg, 603.5 μmol), diphenylmethanimine (142 mg, 7.84 μmol), BINAP (112 mg, 181 μmol), Pd₂(dba)₃ (55.2 mg, 60.3 μmol) and sodium tert-butoxide (86.9 mg, 905 μmol) in toluene (3 mL) was degassed and purged with N₂ for 3 times, and then the mixture was stirred at 80° C. for 16 hrs under N₂ atmosphere. The solution was filtered and concentrated in vacuum. The residue was purified by flash chromatography (ISCO®; 4 g SepaFlash® Silica Flash Column, petroleum ether/EtOAc with EtOAc from 0˜50%, 5 mL/min, 254 mn) to afford 3-(benzhydrylideneamino)pyrazolo[1,5-a]pyridine-5-carbonitrile (Intermediate 118-d) as a yellow oil (100 mg, 51.4% yield). ¹H NMR (400 MHz, DMSO-d₆) δ ppm 6.89-7.95 (m, 2H), 8.53-8.84 (m, 1H), 10.13-10.27 (m, 1H), 10.44-10.64 (m, 1H); LCMS (ESI) m/z 323.4 [M+]⁺.

### Step 5

A mixture of 3-(benzhydrylideneamino)pyrazolo[1,5-a]pyridine-5-carbonitrile (Intermediate 118-d) (90.0 mg, 279 μmol) in HCP/MeOH (4 M, 1.5 mL) was degassed and purged with N₂ for 3 times, and then the mixture was stirred at 25° C. for 3 hrs under N₂ atmosphere. The solution was filtered and extracted with ethyl acetate (20 mL×2). The combined organic phase was washed with brine (20 mL), dried with anhydrous Na₂SO₄, filtered and concentrated in vacuum Compound 3-aminopyrazolo[1,5-a]pyridine-5-carbonitrile (Intermediate 118-e) (40 mg, crude) was obtained as a black solid. LCMS (ESI) m/z 158.8 [M+H]⁺.

### Step 6

A mixture of 3-aminopyrazolo[1,5-a]pyridine-5-carbonitrile (Intermediate 118-e) (40 mg, 253 μmol), 2-chloro-N-methyl-5-(trifluoromethyl)pyrimidin-4-amine (Intermediate 111-a) (53.5 mg, 252 μmol) and HCl (1 M, 252 μL) in i-PrOH (5 mL) was stirred at 80° C. for 2 hrs. The reaction was purified by prep-HPLC (YMC-Actus Triart C18 150×30 mm×5 μm; mobile phase: [water (HCl)-ACN]; B %: 5%-45%, 9 mins), to provide the title compound as a yellow solid (15.1 mg, 17.9% yield). ¹H NMR (400 MHz, DMSO-dt) δ (ppm 2.84-2.98 (m, 3H), 7.04-7.15 (m, 1H), 7.44-7.62 (m, 1H), 8.16-8.32 (m, 1H), 8.52-8.68 (m, 2H), 8.72-8.80 (m, 1H), 10.09-10.39 (m, 1H); LCMS (ESI) m/z 334.0 [M+H]⁺.

### Example 119

### Synthesis of 3-((4-(methylamino)-5-(trifluoromethyl) pyrimidin-2-yl)amino)pyrazolo[1,5-a]pyridine-6-carbonitrile

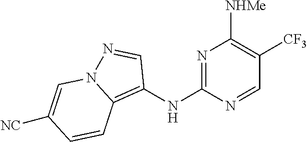 [SMILES: CNc1nc(Nc2cnn3cc(C#N)ccc23)ncc1C(F)(F)F]

### Step 1

A mixture of 6-bromopyrazolo[1,5-a]pyridin-3-amine (150 mg, 707 μmol), 2-chloro-N-methyl-5-(trifluoromethyl) pyrimidin-4-amine (150 mg, 707 μmol) and HO (1 M, 707 μL) in i-PrOH (3 mL) was stirred at 80° C. for 1 hr. The reaction mixture was concentrated under reduced pressure to provide the crude product N²-(6-bromopyrazolo[1,5-a]pyridin-3-yl)-N⁴-methyl-5-(trifluoromethyl)pyrimidine-2,4-diamine (Intermediate 119-a) as a white solid, which was used into the next step without further purification (200 mg, 73.1% yield). ¹H NMR (400 MHz, DMSO-d₆) 2.83-2.94 (m, 3H), 7.38 (br d, J=9.38 Hz, 1H), 7.85 (br d, J=8.88 Hz, 1H), 8.38 (br s, 2H), 8.98-9.08 (m, 1H), 10.99-11.30 (m, 1H).

### Step 2

A mixture of N²-(6-bromopyrazolo[1,5-a]pyridin-3-yl)-N⁴-methyl-5-(trifluoromethyl)pyrimidine-2,4-diamine (Intermediate 119-a) (100 mg, 258 μmol), ditert-butyl-[2,3,4,5-tetramethyl-6-(2,4,6-triisopropylphenyl)phenyl]phosphane (26.1 mg, 54.2 μmol), [2-(2-aminophenyl)phenyl]-chloro-palladium; dicyclohexyl-[3-(2,4,6-triisopropylphenyl) phenyl] phosphane (42.7 mg, 54.2 μmol) tetrapotassium; hexacyanoiron(4-); trihydrate (179 mg, 424 μmol) and potassium acetate (78.1 mg, 796 μmol) in CPME/H₂O (5:1, 5 mL) was degassed and purged with N₂ for 3 tines, and then the mixture was stirred at 120° C. for 12 hrs under N₂ atmosphere. The reaction was purified by prep-HPLC (YMC C18; mobile phase: [water(HCl)-ACN]; B %: 13%-53%, 9 min) to provide the title compound as a yellow solid (7.7 mg, 8.95% yield). ¹H NMR (400 MHz, DMSO-d₆) 2.88 (br s, 3H), 7.40 (br d, J=9.3 Hz, 1H), 7.96 (br s, 2H), 8.33 (br s, 1H), 8.66 (br s, 1H), 9.52 (br s, 1H), 10.68 (br s, 1H); LCMS (ESI) m/z 334.0 [M+H]⁺.

### Example 120

### Synthesis of N⁴-methyl-5-(trifluoromethyl)-N²-(5-vinyl-1H-indazol-6-yl)pyrimidine-2,4-diamine

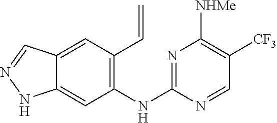 [SMILES: C=Cc1cc2cn[nH]c2cc1Nc1ncc(C(F)(F)F)c(NC)n1]

### Step 1

To a solution of 5-bromo-6-nitro-1H-indazole (1 g, 4.13 mmol), NaH (198 mg, 4.96 mmol, 60% purity) and SEM-Cl (758 mg, 4.54 mmol) in THF (20 mL) was degassed and purged with N₂ for 3 times, and then the mixture was stirred at −60-25° C. for 3 hrs under N₂ atmosphere. The solution was quenched by H₂O (10 mL), extracted with EtOAc (20 mL×2), the combined organic layers were dried over Na₂SO₄ and concentrated. The residue was purified by flash silica gel chromatography (ISCO®; 12 g SepaFlash® Silica Flash Column, Eluent of 0˜100% Ethyl acetate/Petroleum ethergradient @ 12 mL/min) to furnish 2-[(5-bromo-6-nitro-indazol-1-yl)methoxy]ethyl-trimethyl-silane (Intermediate 120-a) as a yellow solid (1 g, 65% yield). LCMS (ESI) m/z 374.1 [M+H]⁺.

### Step 2

To a solution of 2-[(5-bromo-6-nitro-indazol-1-yl)methoxy]ethyl-trimethyl-silane (Intermediate 120-a) (500 mg, 1.34 mmol), 4,4,5,5-tetramethyl-2-vinyl-1,3,2-dioxaborolane (207 mg, 1.34 mmol), Pd(dppf)Cl₂ (49.1 mg, 67 μmol) and K₂CO₃ (557 mg, 4.03 mmol) in dioxane/H₂O (10:1, 10 mL) was degassed and purged with N₂ for 3 times, and then the mixture was stirred at 100° C. for 12 hrs under N₂ atmosphere. The reaction was purified by flash silica gel chromatography (ISCO®; 4 g SepaFlash® Silica Flash Column, Eluent of 0-30% Ethyl acetate/Petroleum ethergradient @ 12 mL/min) to give trimethyl-[2-[(6-nitro-5-vinyl-indazol-1-yl)methoxy]ethyl]silane (Intermediate 120-b) as a yellow solid (240 mg, 56.0% yield). ¹H NMR (400 MHz, methanol-d₄) δ ppm −0.11-0.04 (m, 9H), 0.76-1.06 (m, 2H), 3.53-3.69 (m, 2H), 4.85-4.90 (m, 1H), 5.38-5.43 (m, 1H), 5.81-5.84 (m, 2H), 6.88-7.19 (m, 1H), 8.09 (s, 1H), 8.22 (d, J=0.73 Hz, 1H), 8.29 (s, 1H).

### Step 3

To a solution of trimethyl-[2-[(6-nitro-5-vinyl-indazol-1-yl)methoxy]ethyl]silane (Intermediate 120-b) (240 mg, 814 μmol), tetrabutylammonium; fluoride; trihydrate (1 M in THF, 814 μL) in THF (3 mL) was stirred at 60° C. for 12 hrs. The reaction was concentrated under reduced pressure to give 6-nitro-5-vinyl-1H-indazole (Intermediate 120-c) as a white solid (100 mg, 60.0% yield), which was used for the next step without any purification. LCMS (ESI) m/z 190 [M+H]⁺.

### Step 4

To a solution of 6-nitro-5-vinyl-1H-indazole (Intermediate 120-c) (100 mg, 529 μmol) in EtOH (6 mL) and H₂O (2 mL) was added Fe powder (295 mg, 5.29 mmol) and NH₄Cl (283 mg, 5.29 mmol); then the mixture was stirred at 50° C. for 3 hrs. The mixture was filtered and concentrated to give 5-vinyl-1H-indazol-6-amine (Intermediate 120-d) was obtained as a yellow oil (100 mg, crude). ¹H NMR (400 MHz, DMSO-d₆) δ ppm 5.05 (s, 2H), 5.17-5.25 (m, 1H), 5.20 (dd, J=10.8, 1.5 Hz, 1H), 5.52-5.67 (m, 2H), 6.60 (s, 1H), 6.93 (dd, J=17.2, 10.9 Hz, 1H), 7.61 (s, 1H), 8.21 (s, 1H).

### Step 5

A solution of 2-chloro-N-methyl-5-(trifluoromethyl)pyrimidin-4-amine (Intermediate 1-a) (133 mg, 628 μmol), 1M HCl (628 μL) and 5-vinyl-1H-indazol-6-amine (Intermediate 120-d) (100 mg, 628 μmol) in IPA (3 mL) stirred at 80° C. for 4 hrs. The reaction was purified by Pre-HPLC (column: YMC Triart 30×150 mm×7 μm; mobile phase: [water (HCl)-ACN]; B %: 13%-53%, 9 min), to provide the title compound as a light yellow solid (14 mg, 6.33% yield). ¹H NMR (400 MHz, DMSO-d₆) δ ppm 2.86 (br s, 3H), 5.28 (d, J=12.0 Hz, 1H), 5.72-5.88 (m, 1H), 6.94 (dd, J=17.3, 10.8 Hz, 1H), 7.72 (s, 1H), 8.10 (d, J=16.3 Hz, 2H), 8.16-8.37 (m, 1H), 8.24 (br s, 1H), 10.05-10.44 (m, 1H), 12.75-13.39 (m, 1H); LCMS (ESI) m/z 335.1 [M+H]⁺.

### Example 121

### Synthesis of N²-imidazo[1,2-a]pyridin-5-yl-N⁴-methyl-5-(trifluoromethyl)pyrimidine-2,4-diamine

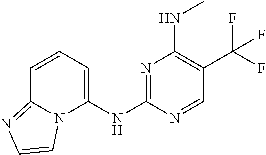 [SMILES: CNc1nc(Nc2cccc3nccn23)ncc1C(F)(F)F]

A solution of imidazo[1,2-a]pyridin-5-amine (100 mg, 751 μmol), 2-chloro-N-methyl-5-(trifluoromethyl)pyrimidin-4-amine (158 mg, 751 μmol) ang HCl (1 M, 751 μL) in i-PrOH (10 mL), then the mixture was stirred at 80° C. for 4 hrs. The reaction was purified by prep-HPLC (column: YMC-Actus Triart C18 150×30 mm×5 μm; mobile phase: [water (HCl)-ACN]; B %: 12%-52%, 9 min), to provide the title compound as a white solid (6.8 mg, 2.94% yield). ¹H NMR (400 MHz, methanol-d₄) δ ppm 3.10-3.22 (m, 3H), 6.83 (d, J=8.03 Hz, 1H), 8.02 (t, J=8.53 Hz, 1H), 8.22 (d, J=2.76 Hz, 1H), 8.42 (d, J=8.78 Hz, 1H), 8.56 (s, 1H), 8.81 (d, J=3.01 Hz, 1H). LCMS (ESI) m/z 309.4 [M+]⁺.

### Example 122

### Synthesis of N2-(5-ethyl-1H-indazol-6-yl)-N4-methyl-5-(trifluoromethyl)pyrimidine-2,4-diamine

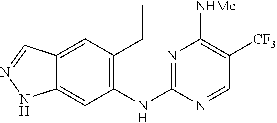 [SMILES: CCc1cc2cn[nH]c2cc1Nc1ncc(C(F)(F)F)c(NC)n1]

### Step 1

To a solution of 6-nitro-5-vinyl-1H-indazole (Intermediate 120-c) (100 mg, 0.53 mmol, 1 eq) in MeOH (5 mL) was added Pd/C (56.0 mg, 10 wt % Pd with 50 wt % water) under N₂ atmosphere. The suspension was degassed and purged with H₂ for 3 times. The mixture was stirred under H₂ (50 Psi) at 30° C. for 16 hrs. The solution was filtered and concentrated to give a 5-ethyl-1H-indazol-6-amine (Intermediate 122-a) (70 mg, 82.1%) as a yellow solid. ¹H NMR (400 MHz, DMSO-d₆) δ ppm 1.06 (t, J=7.0 Hz, 2H), 1.18 (t, J=7.4 Hz, 3H), 4.97 (s, 1H), 4.90 (s, 1H), 5.34-5.64 (m, 1H), 5.52 (dd, J=9.9, 2.1 Hz, 1H), 6.58 (s, 1H), 7.22 (s, 1H), 8.10 (s, 1H).

### Step 2

To a solution of 2-chloro-N-methyl-5-(trifluoromethyl)pyrimidin-4-amine (Intermediate 1-a) (91.9 mg, 0.43 mmol, 1 eq) and 5-ethyl-1H-indazol-6-amine (Intermediate 122-a) (70 mg, 0.43 mmol) in IPA (3 ml) was added 1M HCl (0.43 mL). The mixture was stirred at 80° C. for 4 hrs. The reaction was purified by Pre-HPLC (column: Boston Prine C18 150×30 mm×5 μm; mobile phase: [water (ammonia hydroxide v/v)-ACN]; B %: 32%-62%, 9 min) to give the title compound as yellow solid (2 mg, 1.37%). ¹H NMR (400 MHz, CD₃OD) δ ppm 1.30 (t, J=7.5 Hz, 3H), 2.78-2.86 (m, 1H), 2.78-2.84 (m, 1H), 3.00 (s, 3H), 7.64 (s, 1H), 7.97 (s, 1H), 8.02-8.08 (m, 2H); LCMS (ESI) m/z 337.1 [M+H]⁺.

### Example 123

### Synthesis of N⁴-methyl-5-(trifluoromethyl)-N²-[5-(trifluoromethyl)-1H-indazol-6-yl]pyrimidine-2,4-diamine

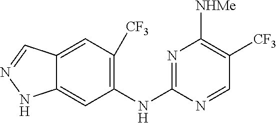 [SMILES: CNc1nc(Nc2cc3[nH]ncc3cc2C(F)(F)F)ncc1C(F)(F)F]

### Step 1

A solution of 5-bromo-6-nitro-1H-indazole (1 g, 1.13 mmol, 1 eq), 3,4-dihydro-2H-pyran (521 mg, 6.20 mmol, 1.5 eq) and PPTS (156 mg, 0.62 mmol, 0.15 eq) in THF (15 mL) was degassed and purged with N₂ for 3 times, and then the mixture was stirred at 25° C. for 3 hrs under N₂ atmosphere. The reaction was quenched by water (10 mL), extracted with EA (15 mL), dried over with Na₂SO₄ and concentrated under reduced pressure to give 5-bromo-6-nitro-1-tetrahydropyran-2-yl-indazole (Intermediate 123-a) as a yellow oil (1 g, 74.2%). LCMS (ESI) m/z 328.1 [M+H]⁺.

### Step 2

A mixture of 5-bromo-6-nitro-1-tetrahydropyran-2-yl-indazole (Intermediate 123-a) (500 mg, 1.53 mmol, 1.0 eq), methyl 2,2-difluoro-2-fluorosulfonyl-acetate (1.47 g, 7.67 mmol, 5.0 eq) and CuI (729 mg, 3.83 mmol, 2.5 eq) in DMF (10 mL) and HMPA (10 mL) was de-gassed and then heated to 80° C. for 16 hrs under N₂ atmosphere. The reaction mixture was poured into H₂O (30 mL). The resulting mixture was extracted with ethyl acetate (50 mL×2). The organic phase was washed with H₂O (50 mL×2), brine (50 mL), dried over anhydrous Na₂SO₄, and concentrated in vacuum. The residue was purified by flash chromatography (ISCO®; 20 g SepaFlash® Silica Flash Column, petroleum ether/EtOAc with EtOAc from 0-30%, 40 mL/min, 254 mn) to afford 6-nitro-5-(trifluoromethyl)-1H-indazole (Intermediate 123-b) as yellow solid (80 mg, 22.6%). ¹H NMR (400 MHz, DMSO-d₆) δ ppm 8.45 (br d, J=9.8 Hz, 2H), 8.58 (s, 1H), 14.15 (br s, 1H).

### Step 3

A mixture of 6-nitro-5-(trifluoromethyl)-1H-indazole (Intermediate 123-b) (50 mg, 0.21 mmol, 1.0 eq), Fe powder (121 mg, 2.16 mmol, 10.0 eq) and NH₄Cl (1161 mg, 2.16 mmol, 10 eq) in EtOH/H₂O (3:1, 4 mL) heated to 50° C. for 3 hrs under N₂. The reaction mixture was filtered and concentrated to give 5-(trifluoromethyl)-1H-indazol-6-amine (Intermediate 123-c) as a yellow oil (50 mg, 100%). LCMS (ESI) m/z 201.7 [M+H]⁺.

### Step 4

To a solution of 2-chloro-N-methyl-5-(trifluoromethyl)pyrimidin-4-amine (intermediate 1-a) (52.6 mg, 0.25 mmol) and 5-(trifluoromethyl)-1H-indazol-6-amine (Intermediate 123-c) (50 mg, 0.25 mmol) in IPA (3 mL) was added 1M HCl (24.9 μL, 0.025 mmol, 0.1 eq). The mixture was stirred at 80° C. for 4 hrs. The reaction was purified by Pre-HPLC (column: Boston Prime C18 150×30 mm×5 μm; mobile phase: [water (ammonia hydroxide v/v)-ACN]; B %: 32%-62%, 9 min) to provide the title compound as a yellow solid (2.1 mg, 2.3%). ¹H NMR (400 MHz, CD₃OD) δ ppm 2.86-3.03 (m, 1H), 2.94-3.03 (m, 3H), 8.06 (s, 1H), 8.18 (s, 1H), 8.23 (d, J=5.8 Hz, 2H); LCMS (ESI) m/z 377.1 [M+H]⁺.

### Example 124

### Synthesis of N⁴-methyl-N²-([1,2,4]triazolo[4,3-a]pyridin-5-yl)-5-(trifluoromethyl) pyrimidine-2,4-diamine

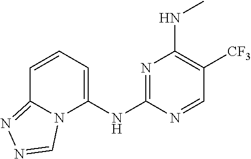 [SMILES: CNc1nc(Nc2cccc3nncn23)ncc1C(F)(F)F]

### Step 1

To a solution of 5-bromo-[1,2,4]triazolo[4,3-a]pyridine (500 mg, 2.52 mmol) and NH₂Boc (355 mg, 3.03 mmol, 1 eq) in dioxane (10 mL) was added K₃PO₄ (1.61 g, 7.57 mmol, 1 eq) and XPhosPdG3 (214 mg, 0.25 mmol, 0.1 eq). The mixture was stirred at 100° C. for 16 hrs under N₂ atmosphere. The resulting mixture was filtered and concentrated. The residue was purified by flash chromatography (ISCO®; 12 g SepaFlash® Silica Flash Column, petroleum ether/EtOAc with EtOAc from 0˜100%, 20 mL/min, 254 mn) to give tert-butyl N-([1,2,4]triazolo[4,3-a]pyridin-5-yl)carbamate (Intermediate 124-a) as a yellow solid (95 mg, 16.1%). ¹H NMR (400 MHz, CDCl₃) δ ppm 1.47 (s, 10H), 6.98 (br d, J=7.3 Hz, 1H), 7.23-7.29 (nm, 1H) 7.45 (br s, 1H), 7.54 (br d, J=9.1 Hz, 1H), 8.79-9.12 (m, 1H).

### Step 2

A solution of tert-butyl N-([1,2,4]triazolo[4,3-a]pyridin-5-yl)carbamate (Intermediate 124-a) (95 mg, 0.40 mmol) in 4M HCl/MeOH (3 mL) was stirred at 25° C. for 2 hrs under N₂ atmosphere. The solution was concentrated to give [1,2,4]triazolo[4,3-a]pyridin-5-amine (Intermediate 124-b) as a yellow solid (50 mg, crude).

### Step 3

To a solution of [1,2,4]triazolo[4,3-a]pyridin-5-amine (Intermediate 124-b) (45 mg, 0.26 mmol) and 2-chloro-N-methyl-5-(trifluoromethyl)pyrimidin-4-amine (Intermediate 1-a) (55.8 mg, 0.26 mmol, 1 eq) in IPA (3 mL) was added 1M HCl (26.4 μL, 26.4 μmol, 0.1 eq). The mixture was stirred at 80° C. for 4 hrs. The reaction was purified by Pre-HPLC (column: Boston Prime C18 150×30 mm×5 μm; mobile phase: [water (HCl)-ACN]; B %: 0%-30%, 9 min) to give the title compound as a yellow solid (12.5 mg, 15.3%). ¹H NMR (400 MHz, DMSO-d₆) δ ppm 3.08 (d, J=4.3 Hz, 3H). 6.82 (d, J=8.5 Hz, 1H), 7.88-8.23 (m, 3H), 8.66 (s, 1H), 9.09 (s, 2H), 10.25 (s, 1H); LCMS (ESI) m/z 309.9 [M+H]⁺.

### Example 1

### Synthesis of 4-methyl-N2(7-methylpyrrolo[2,3-d]pyrimidin-5-yl)-5-(trifluoromethyl) pyrimidine-2,4-diamine

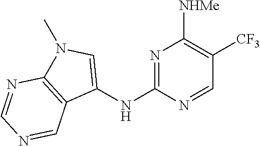 [SMILES: CNc1nc(Nc2cn(C)c3ncncc23)ncc1C(F)(F)F]

### Step 1

To a solution of 5-bromo-7H-pyrrolo[2,3-d]pyrimidine (1 g, 5.05 mmol, 1 eq) in THF (20 mL) was added NaH (403 mg, 10.1 mmol, 60% purity, 2 eq) and MeI (716 mg, 5.05 mmol, 1 eq) was added dropwise at 0° C. The mixture was stirred at 20° C. for 2 hr. The reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure. The residue was purified by flash silica gel chromatography (ISCO®; 20 g SepaFlash® Silica Flash Column, Eluent of 0-50% petroleum ether/EtOAc @ 35 mL/min) to give 5-bromo-7-methyl-pyrrolo[2,3-d]pyrimidine (Intermediate 125-a) was obtained as a white solid (1.1 g, 48.2%). ¹H NMR (400 MHz, DMSO-d₆) δ ppm 3.82 (s, 3H), 7.88 (s, 1H), 8.90 (d, J=10.61 Hz, 2H). LCMS (ESI) m/z 211.9 [M+H]⁺.

### Step 2

A mixture of 5-bromo-7-methyl-pyrrolo[2,3-d]pyrimidine (Intermediate 125-a) (1 g, 4.72 mmol, 1 eq), diphenylmethanimine (940 mg, 5.19 mmol, 1.1 eq), Pd₂(dba)₃ (863 mg, 0.94 mmol, 0.2 eq), BINAP (1.17 g, 1.89 mmol, 0.4 eq) and Cs₂CO₃ (4.61 g, 14.1 mmol, 3 eq) in DMSO (10 mL) was degassed and purged with N₂ for 3 times, and then the mixture was stirred at 100° C. for 16 hrs under N₂ atmosphere. The reaction mixture was diluted with H₂O (30 mL) and extracted with EtOAc (30 mL×3). The combined organic layers were washed with brine (30 mL×2), dried with anhydrous Na₂SO₄, filtered and concentrated under reduced pressure to give a residue. The residue was purified by flash silica gel chromatography (ISCO®; 40 g SepaFlash® Silica Flash Column, Eluent of 0-40% petroleum ether/EtOAc @ 35 mL/min) to give N-(7-methylpyrrolo[2,3-d]pyrimidin-5-yl)-1,1-diphenyl-methanimine (Intermediate 125-b) as brown oil (400 mg 16.8%). LCMS (ESI) m/z 313.1 [M+H]⁺.

### Step 3

A mixture of 2-chloro-N-methyl-5-(trifluoromethyl)pyrimidin-4-amine (Intermediate 1-a) (237 mg, 1.12 mmol, 1 eq), N-(7-methylpyrrolo[2,3-d]pyrimidin-5-yl)-1,1-diphenyl-methanimine (350 mg, 1.12 mmol, 1 eq) (Intermediate 125-b) and HCl (1 M, 50.0 μL) in IPA (1 mL) was stirred at 100° C. for 1.5 hrs. The reaction was purified by flash silica gel chromatography (ISCO®; 12 g SepaFlash® Silica Flash Column, Eluent of 0˜100% petroleum ether/EtOAc @ 35 mL/min) to provide the title compound as a white solid (14.72 mg, 3.9%). ¹H NMR (400 MHz, DMSO-d₆) δ ppm 2.98 (br d, J=3.70 Hz, 3H), 3.80 (s, 3H), 7.20 (br d, J=10.37 Hz, 1H), 7.91 (s, 1H), 8.19 (s, 1H), 8.76 (s, 1H), 9.33 (s, 1H), 9.64-10.30 (m, 1H); LCMS (ESI) m/z 324.1 [M+H]⁺.

### Example 126

### Synthesis of N⁴-methyl-N²-pyrrolo[1,2-a]pyrazin-6-yl-5-(trifluoromethyl)pyrimidine-2,4-diamine

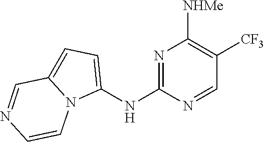 [SMILES: CNc1nc(Nc2ccc3cnccn23)ncc1C(F)(F)F]

### Step 1

To a solution of 2-bromo-1,1-dimethoxy-ethane (1.94 g, 11.49 mmol, 2 eq) in DMF (10 mL) was added 5-bromo-1H-pyrrole-2-carbaldehyde (1 g, 5.75 mmol, 1 eq) and Cs₂CO₃ (3.75 g, 11.49 mmol, 2 eq) at 20° C. The reaction mixture was stirred at 90° C. for 16 hrs. The reaction mixture was quenched with H₂O (100 mL) and extracted with EtOAc (100 mL×2). The combined organic layers were dried over Na₂SO₄, filtered and concentrated under reduced pressure. The residue was purified by flash silica gel chromatography (Biotage®; Agela® Flash Column Silica-CS (24 g), Eluent of Petroleum ether to 0% EtOAc in Petroleum ether, gradient (60 mL/min; Wavelength: 254 nm). Compound 5-bromo-1-(2,2-dimethoxyethyl)pyrrole-2-carbaldehyde (Intermediate 126-a) as a brown oil (1.36 g, 76.74%). ¹H NMR (400 MHz, CDCl₃) δ ppm 3.37 (s, 6H), 4.51-4.54 (m, 2H), 4.57-4.62 (m, 1H), 4.57-4.62 (m, 1H), 6.35 (d, J=4.13 Hz, 1H), 6.94 (d, J=4.25 Hz, 1H), 9.40 (s, 1H).

### Step 2

To a solution of 5-bromo-1-(2,2-dimethoxyethyl)pyrrole-2-carbaldehyde (Intermediate 126-a) (1 g, 3.24 mmol, 1 eq) in AcOH (30 mL) was added NH₄OAc (4.99 g, 64.80 mmol, 20 eq) at 20° C. The reaction mixture was stirred at 110° C. for 16 hrs. The reaction mixture was quenched with H₂O (100 mL) and extracted with EtOAc (100 mL×2). The combined organic layers were dried over Na₂SO₄, filtered and concentrated under reduced pressure. The residue was purified by flash silica gel chromatography (Biotage®; Agela® Flash Column Silica-CS (24 g), Eluent of Petroleum ether to 0% EtOAc in Petroleum ether, gradient @ 60 mL/min; Wavelength: 254 nm), to provide 6-bromopyrrolo[1,2-a]pyrazine (Intermediate 126-b) as a brown oil (1.36 g, 76.74%). 41 NMR (400 MHz, CDCl₃) δ ppm 6.88-6.91 (m, 1H), 6.92-6.95 (m, 1H), 7.66 (d, J=4.88 Hz, 1H), 7.87 (dt, J=5.00, 1.13 Hz, 1H), 8.77 (s, 1H).

### Step 3

To a solution of 6-bromopyrrolo[1,2-a]pyrazine (intermediate 126-b) (300 mg, 1.45 mmol, 1 eq.) in toluene (6 mL) was added diphenylmethanimine (551.89 mg, 2.89 mmol, 2 eq.), (1E,4E)-1,5-diphenylpenta-1,4-dien-3-one; palladium (87.55 mg, 0.14 mmol, 0.1 eq.), sodium; 2-methylpropan-2-olate (292.65 mg, 2.89 mmol, 2 eq.) and [1-(2-diphenylphosphanyl-1-naphthyl)-2-naphthyl]-diphenyl-phosphane (189.62 mg, 0.28 mmol, 0.2 eq) at 20° C. The reaction mixture was stirred at 100° C. for 16 h under N₂. The mixture was quenched with H₂O (50 mL), filtered and extracted with EtOAc (50 mL×2) The organic layers were combined, dried over Na₂SO₄, filtered and concentrated under reduced pressure. The residue was purified by flash silica gel chromatography (Biotage®; Agela® Flash Column Silica-CS (40 g), Eluent of Petroleum ether to 30% EtOAc in Petroleum ether, gradient @ 60 mL/min; Wavelength: 254 nm) to provide 1,1-diphenyl-N-pyrrolo[1,2-a]pyrazin-6-yl-methanimine (Intermediate 126-c) as a brown oil (447 mg, 50.92%). ¹H NMR (400 MHz, CDCl₃) δ ppm 5.38 (d, J=4.63 Hz, 1H), 6.53 (d, J=4.63 Hz, 1H), 7.28-7.32 (m, 2H), 7.39-7.47 (m, 3H), 7.54-7.59 (m, 3H), 7.65 (d, J=4.88 Hz, 1H), 7.87 (dd, J=8.00, 1.63 Hz, 2H), 8.51 (d, J=4.88 Hz, 1H), 8.68 (s, 1H); LCMS (ESI) m/z 298.2 [M+H]⁺.

### Step 4

A solution of 1,1-diphenyl-N-pyrrolo[1,2-a]pyrazin-6-yl-methanimine (Intermediate 126-c) (290 mg, 0.95 mmol, 1 eq) and HCl (3 M, 3 mL, 9.42 eq) in MeOH (9 mL) was stirred at 20° C. for 1 hr. The reaction was concentrated to remove organic solvent to provide pyrrolo[1,2-a]pyrazin-6-amine (Intermediate 126-d) was obtained as a yellow solid (303 mg, 99.06%). ¹H NMR (400 MHz, DMSO-d₆) δ ppm 6.67 (d, J=4.88 Hz, 1H), 7.21 (t, J=5.07 Hz, 1H), 7.52 (d, J=4.75 Hz, 1H), 7.82 (br t, J=4.38 Hz, 1H), 8.52 (d, J=6.25 Hz, 1H), 12.21 (br s, 1H).

### Step 5

To a solution of pyrrolo[1,2-a]pyrazin-6-amine (Intermediate 126-d) (100 mg, 0.31 mmol, 1 eq) in DMSO (3 mL, 99% purity) was added 2-chloro-N-methyl-5-(trifluoromethyl)pyrimidin-4-amine (Intermediate 1-a) (69.59 mg, 0.31 mmol, 1 eq) and TEA (126.48 mg, 1.25 mmol, 4 eq) at 20° C. The reaction mixture was stirred at 20° C. for 16 hrs. The mixture was quenched with H₂O (50 mL), filtered and extracted with EtOAc (50 mL×2). The organic layers were combined, dried over Na₂SO₄, filtered and concentrated under reduced pressure. The crude product was prep-HPLC (Instrument: Prep-SDC-670101, Method: Column Welch Xtimate (C18 150×30 mm×5 μm, Condition: water (HCl)-ACN, Begin B: 0, End B: 90, Gradient Time (min): 36, 100% B Hold Time (min): 3, FlowRate (ml/min): 25, Injections: 1, HPLC: 1), to provide the title compound as a brown solid (5 mg, 4.55%). H NMR (400 MHz, DMSO-d₆) δ ppm 3.06 (d, J=4.50 Hz, 3H), 6.90 (d, J=5.25 Hz, 1H) 7.74 (d, J=6.63 Hz, 1H), 7.86 (d, J=5.25 Hz, 1H), 7.98 (br d, J=4.38 Hz, 1H), 8.12 (dd, J=6.50, 1.63 Hz, 1H), 8.52 (s, 1H), 8.63 (s, 2H), 9.25 (d, J=1.50 Hz, 1H); LCMS (ESI) m/z 309.3 [M+H]⁺.

### Example 127

### Synthesis of 4-[[4-(methylamino)-5-(trifluoromethyl)pyrimidin-2-yl]amino]indolin-2-one

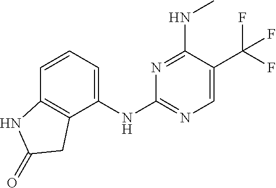 [SMILES: CNc1nc(Nc2cccc3c2CC(=O)N3)ncc1C(F)(F)F]

### Step 1

To a solution of 4-nitroindolin-2-one (505.05 mg, 2.81 mmol, 1 eq.) in methanol (5 mL) in one portion was added Pd/C (297.51 mg, 0.28 mmol, 10% purity, 0.1 eq) at 25° C. under Ar atmosphere. The mixture was stirred at 25° C. under H₂ (15 psi) for 3 hrs at 25° C. t. The mixture was filtered through Celite and washed with Methanol (20 mL×2) and DCM (20 mL×2). The filtrate was concentrated in vacuum and purified by flash silica gel chromatography. (Biotage®; 40 g Agela Silica Flash Column, Eluent of 0-100% Ethyl acetate/Petroleum ether gradient @ 40 mL/min), to provide 4-aminoindolin-2-one (Intermediate 127-a) as a yellow solid (309 mg, 66.88%). ¹H NMR (400 MHz, CD₃OD) δ ppm 3.32 (s, 2H), 6.28 (d, J=7.63 Hz, 1H), 6.4 (dd, J=8.19, 0.69 Hz, 1H), 6.96 (br t, J=7.94 Hz, 1H). LCMS (ESI) m/z 149.3 [M+H]⁺.

### Step 2

To a solution of 4-aminoindolin-2-one (Intermediate 127-a) (100 mg, 0.60 mmol, 1 eq) and 2-chloro-N-methyl-5-(trifluoromethyl)pyrimidin-4-amine (Intermediate 1-a) (135.28 mg, 0.60 mmol, 1 eq) in IPA (3 mL) was added HCl (1 M, 0.60 mmol) at 80° C. for 16 hrs. The reaction was purified by prep-HPLC (column: Welch Xtimate C18 150×30 mm×5 μm; mobile phase: [water (NH₄HCO₃)-ACN]; B %: 0%-90%, 36 min), to provide the title compound as an orange solid (124.4 mg, 62.72%). ¹H NMR (400 MHz, DMSO-d₆) δ ppm 2.86 (s, 3H), 3.50 (s, 2H), 6.55 (d, J=7.63 Hz, 1H), 7.09-7.14 (m, 2H), 7.44 (br d, J: 8.38 Hz, 1H), 8.14 (s, 1H), 9.08 (s, 1H), 10.36 (br s, 1H). ¹F NMR: −60.284 ppm. LCMS (ESI) m/z 324.3 [M+H]⁺.

### Example 128

### Synthesis of N⁴-methyl-N²-(1-methyl-1H-pyrazolo[3,4-d]pyrimidin-3-yl)-5-(trifluoromethyl)pyridine-2,4-diamine

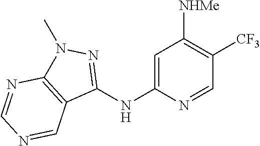 [SMILES: CNc1cc(Nc2nn(C)c3ncncc23)ncc1C(F)(F)F]

### Step 1

To a solution of 3-bromo-1H-pyrazolo[3,4-d]pyrimidine (500 mg, 2.51 mmol, 1.0 eq.) in acetone (5 mL) was added MeI (534.9 mg, 3.77 mmol, 1.5 eq.) and KOH (211.4 mg, 3.77 mmol, 1.5 eq.) at 0° C. The mixture was stirred at 25° C. for 3 hrs. The residue was diluted with H₂O (20 mL) and extracted with EtOAc (30 mL×3). The combined organic layers were washed with brine (20 mL), dried over Na₂SO₄, filtered and concentrated under reduced pressure to give a residue, which was purified by flash chromatography (ISCO®; 12 g SepaFlash® Silica Flash Column, petroleum ether/EtOAc with EtOAc from 0˜20%, 30 mL/min, 254 mn) to give 3-bromo-1-methyl-pyrazolo[3,4-d]pyrimidine (Intermediate 128-a) as a white solid (330 mg, 61.6%). 1H NMR (400 MHz, DMSO-d₆) δ ppm 3.97-4.11 (n, 1H), 3.97-4.11 (m, 2H), 9.10 (s, 1H), 9.25 (s, 1H).

### Step 2

To a solution of 3-bromo-1-methyl-pyrazolo[3,4-d]pyrimidine (Intermediate 128-a) (300 mg, 1.41 mmol, 1.0 eq.) in dioxane (5 mL) was added diphenylmethanimine (331.8 mg, 1.83 mmol, 1.3 eq.), Xantphos Pd G4 (135.5 mg, 0.14 mmol, 0.1 eq.) and Cs₂CO₃ (1.10 g, 3.38 mmol, 2.4 eq.). The mixture was degassed and purged with N₂ for three times. The mixture was stirred at 90° C. for 2 hrs under N₂ atmosphere. The residue was diluted with H₂O (10 mL) and extracted with EtOAc (15 mL×3). The combined organic layers were washed with brine (15 mL), dried over Na₂SO₄, filtered and concentrated under reduced pressure to give a residue, which was purified by flash chromatography (ISCO®; 12 g SepaFlash® Silica Flash Column, petroleum ether/EtOAc with EtOAc from 042%, 30 mL/min, 254 mn) to provide N-(1-methylpyrazolo[3,4-d]pyrimidin-3-yl)-1,1-diphenyl-methanimine (Intermediate 128-b) as yellow oil (400 mg, 67.9%).

### Step 3

A solution of N-(1-methylpyrazolo[3,4-d]pyrimidin-3-yl)-1,1-diphenyl-methanimine (Intermediate 128-b) (380 mg, 0.91 mmol, 1.0 eq.) in HCl/dioxane (4 M, 5 mL) was stirred at 25° C. for 0.5 hrs. The reaction was diluted with H₂O (10 mL) and extracted with EtOAc (15 mL×3). The combined aqueous phase was basified at 0° C. with aq. NaHCO)₃ to pH=9-10 and extracted with EtOAc (20 mL×3). Then the combined organic layers were washed with brine (20 mL), dried by Na₂SO₄, filtered and concentrated under reduced pressure to give 1-methylpyrazolo[3,4-d]pyrimidin-3-amine (Intermediate 128-c) as a light yellow solid (110 mg, 81%). LCMS (ESI) m/z 150.1 [M+H]⁺.

### Step 4

To a solution of 1-methylpyrazolo[3,4-d]pyrimidin-3-amine (Intermediate 128-c) (80 mg, 0.54 mmol) in dioxane (2 mL, 1.0 eq) was added 2-chloro-N-methyl-5-(trifluoromethyl)pyridin-4-amine (Intermediate 1-a) (124.2 mg, 0.59 mmol, 1.1 eq), K₂CO₃ (222.4 mg, 1.61 mmol, 3.0 eq), Pd₂(dba)₃ (196.4 mg, 0.21 mmol, 0.4 eq) and XPhos (102.3 mg, 0.21 mmol, 0.4 eq). The mixture was degassed and purged with N₂ for three tines. The mixture was stirred at 100° C. for 12 hrs under N₂ atmosphere. The reaction mixture was concentrated under reduced pressure to give a residue, which was purified by flash chromatography (ISCO®; 20 g SepaFlash® Silica Flash Column, petroleum ether/EtOAc with EtOAc from 0˜80%, 30 mL/min, 254 mn) to provide the title compound as a white solid (23.36 mg, 13.4%). ¹H NMR (400 MHz, DMSO-d₆) δ ppm 2.85 (d, J=4.53 Hz, 3-1), 3.94 (s, 3H), 6.44 (br d, J=4.53 Hz, 1H, 7.14 (s, 1H), 8.01-8.17 (m, 1H), 8.93 (s, 1H), 9.57 (s, 1H), 10.41 (s, 1H); ¹⁹F NMR (377 MHz, DMSO-d₆) δ ppm −59.97 (s, 3F); LCMS (ESI) n/z 324.0 [M+H]⁺.

### Example 129

### Synthesis of N4-methyl-N2-[5-(trifluoromethoxy)-1H-indazol-6-yl]-5-(trifluoromethyl) pyrimidine-2,4-diamine

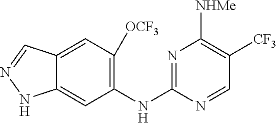 [SMILES: CNc1nc(Nc2cc3[nH]ncc3cc2OC(F)(F)F)ncc1C(F)(F)F]

### Step 1

To a mixture of 3-bromo-4-(trifluoromethoxy)aniline (12 g, 46.87 mmol, 1.0 eq.) in DCE (200 mL) was added NIS (10.56 g, 46.87 mmol, 1.0 eq.) and TsOH (12.12 g, 70.38 mmol, 1.5 eq.) at 20° C. The reaction mixture was stirred at 45° C. for 16 hrs. The reaction mixture was quenched with H₂O (100 mL) and extracted with DCM (80 mL×3). The combined organic layers were dried over Na₂SO₄, filtered and concentrated under reduced pressure to give the crude product. The residue was purified by flash silica gel chromatography (Biotage®; Agela® Flash Column Silica-CS (330 g), Eluent of Petroleum ether to 8% Ethyl acetate in Petroleum ether, gradient @ 80 mL/min), to provide 5-bromo-2-iodo-4-(trifluoromethoxy)aniline (Intermediate 129-a) as red oil (11 g, 29.80%). ¹H NMR (400 MHz, CDCl₃) δ ppm 3.80-4.55 (m, 2H), 6.96 (s, 1H), 7.54 (s, 1H); LCMS (ESI) m/z 381.9 [M+H]⁺.

### Step 2

To a mixture of 5-bromo-2-iodo-4-(trifluoromethoxy)aniline (Intermediate 129-a) (11 g, 27.94 mmol, 1.0 eq) in dioxane (220 mL) was added 2,4,6-trimethyl-1,3,5,2,4,6-trioxatriborinane (5.26 g, 41.87 mmol, 1.5 eq.), CS₂CO₃ (27.31 g, 83.81 mmol, 3.0 eq.) and palladium; triphenylphosphane (6.45 g, 5.58 mmol, 0.02 eq.) at 20° C. under N₂. The mixture was stirred at 100° C. for 16 hrs. The reaction mixture was quenched with H₂O (80 ml) and extracted with ethyl acetate (70 mL×3).

The combined organic layers were dried over Na₂SO₄, filtered and concentrated under reduced pressure to give the crude product, which was purified by flash silica gel chromatography (Biotage®; Agela® Flash Column Silica-CS (330 g), Eluent of Petroleum ether to 15% Ethyl acetate in Petroleum ether, gradient @ 80 mL/min), to provide 5-bromo-2-methyl-4-(trifluoromethoxy)aniline (Intermediate 129-b) as yellow oil (6.7 g, 81.70%). ¹H NMR (400 MHz, CDCl₃) ppm 2.14 (s, 3H), 3.50-4.48 (m, 2H), 6.91 (s, 1H), 7.00 (s, 1H).

### Step 3

To a mixture of 5-bromo-2-methyl-4-(trifluoromethoxy)aniline (Intermediate 129-b) (6.7 g, 22.83 mmol, 1.0 eq.) in CHCl₃ (150 mL) was added KOAc (2.69 g, 27.39 mmol, 1.2 eq.) at 0° C. The mixture was added acetyl acetate (6.99 g, 68.48 mmol, 3.0 eq.) at 20° C. Then added isopentyl nitrite (5.35 g, 45.65 mmol, 2.0 eq.) at 60° C. The mixture was stirred at 60° C. for 16 hrs. The mixture was quenched with H₂O (200 mL) and extracted with DCM (100 mL×2). The combined organic layers were dried over Na₂SO₄, filtered and concentrated under reduced pressure. The residue was purified by flash silica gel chromatography (Biotage®; Agela® Flash Column Silica-CS (330 g), Fluent of Petroleum ether to 4% Ethyl acetate in Petroleum ether, gradient @ 60 mL/min), to provide 1-[6-bromo-5-(trifluoromethoxy)indazol-1-yl]ethanone (Intermediate 129-c) as yellow solid (3.7 g, 47.67%). ¹H NMR (400 MHz, CDCl₃) δ ppm 2.79 (s, 3H), 7.6 (s, 1H), 8.12 (s, 1H), 8.82 (s, 1H).

### Step 4

1-[6-bromo-5-(trifluoromethoxy)indazol-1-yl]ethanone (Intermediate 129-c) (1.3 g, 3.82 mmol, 1.0 eq.) in 1,4-dioxane (30 mL) was added tert-butyl carbamate (1.12 g, 9.56 mmol, 2.5 eq.), XPhos (364.48 mg, 0.76 mmol, 0.02 eq.), dicesium; carbonate (1.87 g, 5.73 mmol, 1.5 eq.) and (1E,4E)-1,5-diphenylpenta-1,4-dien-3-one; palladium (439.62 mg, 0.76 mmol, 0.02 eq.) at 20° C. The reaction mixture was stirred at 100° C. for 16 hrs under N₂ atmosphere. The mixture was quenched with H₂O (90 mL) and extracted with ethyl acetate (60 mL×2). The combined organic layers were dried over Na₂SO₄, filtered and concentrated under reduced pressure. The residue was purified by flash silica gel chromatography (Biotage®; Agela® Flash Column Silica-CS (40 g), Eluent of Petroleum ether to 19% Ethyl acetate in Petroleum ether, gradient @ 70 mL/min), to provide tert-butyl N-[5-(trifluoromethoxy)-1H-indazol-6-yl]carbamate (Intermediate 129-d) as yellow solid (892 mg, 66.93%), ¹H NMR (400 MHz, CDCl₃) δ ppm 1.47 (s, 9H), 4.29-4.64 (m, 2H), 6.96 (s, 1H), 7.62 (s, 1H), 8.02 (s, 1H), 8.41 (s, 1H); LCMS (ESI) m/z 318.1 [M+H].

### Step 5

A solution of tert-butyl N-[5-(trifluoromethoxy)-1H-indazol-6-yl]carbamate (Intermediate 129-d) (150 mg, 0.38 mmol, 1 eq) and TFA (2.38 g, 20.89 mmol, 55 eq.) in DCM (4 mL) was stirred at 20° C. for 6 hrs. The reaction mixture was concentrated under reduced pressure to provide 5-(trifluoromethoxy)-1H-indazol-6-amine (Intermediate 129-e) as a red solid (173 mg, 85.05%). ¹H NMR (400 MHz, DMSO-d₆) δ ppm 6.78 (s, 1H), 7.02 (s, 1H), 7.15 (s, 1H), 7.28 (s, 1H), 7.53 (s, 1H), 7.85 (s, 1H); LCMS (ESI) m/z 217.9 [M+H]⁺.

### Step 6

To a mixture of 5-(trifluoromethoxy)-11H-indazol-6-amine (Intermediate 129-e) (150 mg, 0.30 mmol, 1 eq) in IPA (10 mL) was added 2-chloro-N-methyl-5-(trifluoromethyl)pyrimidin-4-amine (Intermediate 1-a) (68.28 mg, 0.30 mmol, 1 eq) and HCl (1 M, 0.30 mL, 1 eq) at 20° C. The mixture was stirred at 80° C. for 16 hrs. The reaction was purified by Instrument: Prep-SDC-670101, Column: Welch Xtimate C18 150×30 mm×5 μm. Condition: water (HCl)-CAN to afford the title compound as a white solid (10.3 mg, 7.50%). ¹H NMR (400 MHz, DMSO-d₆) δ ppm 2.88 (s, 3H), 7.18 (s, 1H), 7.81 (s, 1H), 8.09 (s, 1H), 8.18 (s, 2H), 8.91 (s, 1H), 13.21 (s, 1H) LCMS (ESI) m/z 393.3 [M+H]⁺.

### Example 130

### Synthesis of N²-(imidazo[1,5-a]pyridin-8-yl)-N⁴-methyl-5-(trifluoromethyl)pyrimidine-2,4-diamine

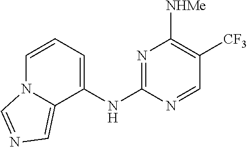 [SMILES: CNc1nc(Nc2cccn3cncc23)ncc1C(F)(F)F]

### Step 1

A mixture of formaldehyde (3.58 g, 44.08 mmol, 1 eq) and acetic acid, ammonia (4.14 g, 53.78 mmol, 1.2 eq.) in AcOH (20 mL) was stirred at 25° C. for 10 minutes before 3-bromopyridine-2-carbaldehyde (1.0 g, 5.38 mmol, 1.2 eq.) was added in portions. And the mixture was stirred at 25° C. for 14 hrs. The residue was diluted with H₂O (30 mL) and extracted with EtOAc (30 mL 3). The combined organic layers were dried over Na₂SO₄, filtered and concentrated under reduced pressure to give a residue, which was purified by flash silica gel chromatography (ISCO®; 40 g SepaFlash® Silica Flash Column, Eluent of 0˜70% Ethylacetate/Petroleum ethergradient @ 45 mL/min), to provide 8-bromoimidazo[1,5-a]pyridine (Intermediate 130-a) as a light yellow solid (350 mg, 32.3%) ¹H NMR (400 MHz, DMSO-d₆) δ ppm 6.60 (m, 1H), 7.12 (d, J=6.80 Hz, 1H), 7.38 (s, 1H), 8.39 (d, J=7.20 Hz, 1H), 8.51 (s, 1H).

### Step 2

To a solution of 8-bromoimidazo[1,5-a]pyridine (intermediate 130-a) (200 mg, 1.00 mmol) and dicesium; carbonate (818.55 mg, 2.51 mmol, 1.0 eq.) in dioxane (6 mL) was added tert-butyl carbamate (176.58 mg, 1.51 mmol, 1.5 eq.), (5-diphenylphosphanyl-9,9-dimethyl-xanthen-4-yl) diphenyl-phosphane (116.29 mg, 0.20 mmol, 0.2 eq.) and (1E,4E)-1,5-diphenylpenta-1,4-dien-3-one; palladium (92.02 mg, 0.10 mmol, 0.1 eq.). The mixture was stirred at 100° C. for 16 hrs under N₂. The residue was diluted with H₂O (20 mL) and extracted with EtOAc (20 mL×3). The combined organic layers were dried over Na₂SO₄, filtered and concentrated under reduced pressure. The residue was purified by flash silica gel chromatography (ISCO®; 12 g SepaFlash® Silica Flash Column, Eluent of 0-100% Ethylacetate/Petroleum ethergradient @ 20 mL/min), to provide tert-butyl N-imidazo[1,5-a]pyridin-8-ylcarbamate (Intermediate 130-b) as a green solid (200 mg, 81.0%). ¹H NMR (400 MHz, DMSO-d₆) δ ppm 1.51 (s, 9H), 6.58 (m, 1H), 7.16 (d, J=7.20 Hz, 1H), 7.72 (s, 1H), 8.01 (d, J=6.80 Hz, 1H), 8.33 (s, 1H), 9.41 (s, 1H).

### Step 3

A solution of tert-butyl N-imidazo[1,5-a]pyridin-8-ylcarbamate (Intermediate 130-b) (180 mg, 0.73 mmol) in d HCl/dioxane (4 M, 5 mL). The mixture was stirred at 25° C. for 4 hrs. The reaction mixture was concentrated under reduced pressure to provide imidazo[1,5-a]pyridin-8-amine (Intermediate 130-c) as a yellow solid (140 mg, 88.0%). ¹H NMR (400 MHz, DMSO-d₆) δ ppm 6.11 (d, J=7.20 Hz, 1H), 6.92 (m, 1H), 7.87 (d, J=6.80 Hz, 1H), 8.15 (s, 1H), 9.52 (d, J=1.60 Hz, 1H).

### Step 4

To a solution of imidazo[1,5-a]pyridin-8-amine (Intermediate 130-c) (74 mg, 0.52 mmol, 1.0 eq.) and 2-chloro-N-methyl-5-(trifluoromethyl)pyrimidin-4-amine (Intermediate 1-a) (117.58 mg, 0.52 mmol, 1.0 eq.) in IPA (3 mL) was added HCl (1 M, 0.52 mL). The mixture was stirred at 80° C. for 16 hrs. The reaction mixture was purified by prep-HPLC (column: Welch Xtimate C18 150×30 mm×5 μm; mobile phase: [water (NH₃H₂O+NH₄HCO₃)-ACN]; B %: 16%-56%, 36 min), to provide the title compound as the yellow solid (15.0 mg, 8.9%). ¹H NMR (400 MHz, DMSO-d₆) δ ppm 2.94 (d, J=4.40 Hz, 3H), 6.64 (m, 1H), 7.26 (m, 1H), 7.62 (d, J=7.20 Hz, 1H), 7.80 (s, 1H), 8.03 (d, J=6.80 Hz, 1H), 8.23 (s, 1H), 8.34 (s, 1H), 9.55 (s, 1H); LCMS (ESI) m/z 309.3 [M+H]⁺.

### Example 131

### Synthesis of 2-[3-[[4-(methylamino)-5-(trifluoromethyl)pyrimidin-2-yl]amino]pyrrolo[2,3-c]pyridin-1-yl]acetonitrile

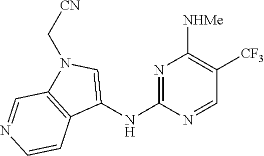 [SMILES: CNc1nc(Nc2cn(CC#N)c3cnccc23)ncc1C(F)(F)F]

### Step 1

To a solution of 3-nitro-1H-pyrrolo [2,3-c]pyridine (1 g, 6.13 mmol) in DMF (10 mL) was added NaH (490 mg, 12.3 mmol) under N₂ atmosphere at 0° C. After 30 mins, 2-bromoacetonitrile (735 mg, 6.13 mmol) was added. The resulting mixture was stirred at 25° C. for 16 hrs. The solution was quenched by NH₄Cl (50 mL), extracted with EtOAc (20 mL), washed with 5% LiCl aq. (80 mL×2), brine (80 mL), dried over Na₂SO₄ and concentrated. The residue was purified by flash chromatography (ISCO®; 12 g SepaFlash® Silica Flash Column, petroleum ether/EtOAc with EtOAc from 0-30%, 18 mL/min, 254 mn) to give 2-(3-nitropyrrolo [2,3-c]pyridin-1-yl)acetonitrile (Intermediate 131-a) as a yellow oil (200 mg, 16.1% yield). ¹H NMR (400 MHz, DMSO-d₆) δ ppm 5.80 (s, 2H), 8.05 (dd, J=5.4, 1.0 Hz, 1H), 8.56 (d, J=5.5 Hz, 1H), 8.98 (s, 1H), 9.21 (s, 1H).

### Step 2

A mixture of 2-(3-nitropyrrolo [2,3-c]pyridin-1-yl)acetonitrile (Intermediate 131-a) (140 mg, 692 μmol), Fe powder (387 mg, 6.92 mmol) and NH₄Cl (370 mg, 6.92 mmol) in EtOH/H₂O (10:1, 10 mL) was stirred at 50° C. for 2 hrs. The resulting mixture was filtered and extracted with EtOAc (15 mL) and concentrated under reduced pressure to give 2-(3-aminopyrrolo[2,3-c]pyridin-1-yl)acetonitrile (Intermediate 131-b) as a yellow solid, which was used for the next step without any further purification (40 mg, 33.6% yield). LCMS (ESI) m/z 173.1 [M+H]⁺.

### Step 3

A solution of 2-(3-aminopyrrolo[2,3-c]pyridin-1-yl)acetonitrile (Intermediate 131-b) (40 mg, 232 mol), 2-cloro-N-methyl-5-(trifluoromethyl)pyrimidin-4-amine (Intermediate 1-a) (49.1 mg, 232 μmol) and HCl (1 M, 464 μL) in i-PrOH (5 mL) was stirred at 50° C. for 2 hrs. The reaction was purified by prep-HPLC (column: Boston Green ODS 150×30 mm×5 μm; mobile phase: [water (HCl)-ACN]; B %: 0%-35%, 9 min), to provide the title compound as a white solid (5.5 mg, 6.82% yield). ¹H NMR (400 MHz, methanol-d₄) δ ppm 2.96-3.17 (m, 3H), 5.82 (s, 2H), 8.36 (br s, 2H), 8.47 (d, J=6.52 Hz, 1H), 8.62-8.73 (m, 1H), 9.54 (s, 1H); LCMS (ESI) m/z 348.0 [M+H]⁺.

### Example 132

### Synthesis of N-(4-cyclopropyl-5-(trifluoromethyl)pyrimidin-2-yl)-1-(cyclopropylsulfonyl)-1H-indazol-4-amine

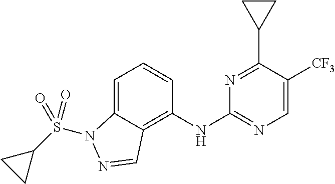 [SMILES: O=S(=O)(C1CC1)n1ncc2c(Nc3ncc(C(F)(F)F)c(C4CC4)n3)cccc21]

### Step 1

To a solution of 2,4-dichloro-5-(trifluoromethyl)pyrimidine (5.00 g, 23.0 mmol, 1.0 eq.), cyclopropanecarboxylic acid (6.00 g 69.7 mmol, 3.0 eq.), ammonia; sulfooxy hydrogen sulfate (26.5 g, 0.116 mol, 5.0 eq.) in MeCN (50 mL)/H₂O (50 mL) was added AgNO₃ (15.7 g, 92.4 mmol, 4.0 eq.) and the reaction mixture was stirred at 60° C. for 12 hr. The reaction mixture was concentrated under reduced pressure to remove solvent. The residue was diluted with water 20 mL and extracted with EtOAc (20 mL×2). The combined organic layers were washed with brine (20 mL×2), dried over anhydrous Na₂SO₄, filtered and concentrated under reduced pressure to give 2-chloro-4-cyclopropyl-5-(trifluoromethyl)pyrimidine (Intermediate 132-a) as brown oil (5.00 g, crude).

### Step 2

A mixture of 1-cyclopropylsulfonylindazol-4-amine (Intermediate 33-c) (100 mg, 0.422 mmol), 2-chloro-4-cyclopropyl-5-(trifluoromethyl)pyrimidine (Intermediate 132-a) (175 mg, 0.786 mmol), 1N HCl/H₂O (750 μL) in IPA (10 mL) was degassed and purged with N₂ for 3 times, and then the mixture was stirred at 80° C. for 4 hr under N₂ atmosphere. The residue was purified by preparative HPLC (Instrument. Gilson GX-281 Liquid Handler, Gilson 322 Pump, Gilson 156 UV Detector; Column: Welch Xtimate C18 150×25 mm×5 μm; Mobile phase A: H₂O with 0.05% NH₃—H₂O (v %); Mobile phase B: ACN; Gradient: B from 70% to 100% in 7.8 min, hold 100% B for 2 min; Flow Rate: 25 mL/min; Column Temperature: 30° C.; Wavelength: 220 nm). The fraction was concentrated under reduced pressure and then lyophilized for overnight. The residue was purified by Instrument: Berger, MULTIGR AM-4; Column: Chiralpak AD 250×30 mm I.D. 3 μm; Mobile phase: supercritical CO₂/EtOH (0.05% NH₃—H₂O, v %)=60/40; Flow Rate: 4 mL/min; Column Temperature: 35° C.; Nozzle Pressure: 100 bar; Nozzle Temperature: 60° C.; Evaporator Temperature: 20° C.; Trimmer Temperature: 25° C.; Wavelength: 220 nm. The fraction was concentrated under reduced pressure and then lyophilized for overnight to provide the title compound as a white solid (20.4 mg, 11.4%). ¹H NMR (400 MHz, CDCl₃) δ ppm 1.04-1.11 (m, 2H), 1.14-1.20 (m, 2H), 1.30 (quin, J=3.8 Hz, 2H), 1.45-1.52 (m, 2H), 2.22-2.31 (m, 1H), 2.75 (tt, J=8.0, 4.6 Hz, 1H), 7.50-7.59 (m, 2H), 7.80 (d, J=8.3 Hz, 1H), 7.90 (d, J=8.0 Hz, 1H), 8.32 (s, 1H), 8.51 (s, 1H); ¹⁹F NMR (377 MHz, CDCl₃) δ ppm −59.75; LCMS (ESI) m/z 424.1 [M+H]⁺.

### Example 133

### Synthesis of N⁴-cyclopropyl-N²-[1-(2-methoxyethyl)indazol-4-yl]-5-(trifluoromethyl) pyrimidine-2,4-diamine

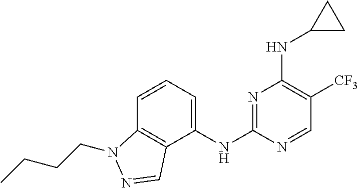 [SMILES: CCCCn1ncc2c(Nc3ncc(C(F)(F)F)c(NC4CC4)n3)cccc21]

### Step 1

To a solution of 4-nitro-1H-indazole (5.00 g, 30.7 mmol, 1.0 eq.) in DMF (200 mL) was added dropwise NaH (2.45 g, 61.3 mmol, 2.0 eq.) at 0° C. After addition, the mixture was stirred at this temperature for 1 h, and then 1-bromo-2-methoxy-ethane (8.52 g, 61.3 mmol, 2.0 eq.) was added dropwise at 0° C. The resulting mixture was stirred at 25° C. for 16 hr. The solution was filtered and extracted with ethyl acetate (20 mL×2). The combined organic phase was washed with brine (20 mL), dried with anhydrous Na₂SO₄, filtered and concentrated in vacuum. The residue was purified by flash chromatography (ISCO®; 80 g SepaFlash® Silica Flash Column, petroleum ether/EtOAc with EtOAc from 0-30%, flow rate=80 mL/min, 254 nm) to provide 1-(2-methoxyethyl)-4-nitro-indazole (Intermediate 133-a) as brown solid (6.76 g, 44.3%). ¹H NMR (400 MHz, DMSO-d₆) δ ppm 3.16 (s, 3H), 3.72-3.83 (m, 2H), 4.02-4.23 (m, 2H), 4.65-4.82 (m, 2H), 7.54-7.72 (m, 1H), 8.09-8.32 (m, 2H), 8.49-8.58 (m, 1H).

### Step 2

To a solution of 1-(2-methoxyethyl)-4-nitro-indazole (Intermediate 133-a) (3.00 g, 13.6 mmol, 1.0 eq.) in MeOH (100 mL) was added Pd/C (2.40 g, 2.26 mmol, 0.2 eq.) (10 wt % Pd with 50 wt % water) and H₂ (15 psi). The mixture was stirred at 25° C. for 16 hr. The solution was filtered and extracted with ethyl acetate (20 mL×2). The combined organic phase was washed with brine (20 mL), dried with anhydrous Na₂SO₄, filtered and concentrated in vacuum. The residue was purified by flash chromatography (ISCO®; 80 g SepaFlash® Silica Flash Column, petroleum ether/EtOAc with EtOAc from 0-30%, flow rate=100 mL/min, 254 nm) to afford 1-(2-methoxyethyl)indazol-4-amine (Intermediate 133-b) as a yellow oil (1.4 g, 54.0%). ¹H NMR (400 MHz, DMSO-d₆) δ ppm 3.14-3.24 (m, 3H), 3.66-3.82 (m, 2H), 4.33-4.48 (m, 2H), 5.50-5.84 (m, 2H), 6.03-6.24 (m, 1H), 6.54-6.77 (m, 1H), 6.90-7.11 (m, 1H), 8.00-8.19 (m, 1H); LCMS (ESI) m/z 192.2 [M+H]⁺.

### Step 3

A mixture of 1-(2-methoxyethyl)indazol-4-amine (Intermediate 133-b) (50.0 mg, 0.262 mmol, 1.0 eq.), 2-chloro-N-cyclopropyl-5-(trifluoromethyl)pyrimidin-4-amine (Intermediate 68-a) (62.1 mg, 0.262 mmol, 1.0 eq.), 1N HCl/H₂O (26.0 μL, 0.0262 nmol, 0.1 eq.) in i-PrOH (5 mL) was degassed and purged with N₂ for 3 times, and then the mixture was stirred at 80° C. for 1 h under N₂ atmosphere. The solution was filtered and extracted with ethyl acetate (20 mL×2). The combined organic phase was washed with brine (20 mL), dried with anhydrous Na₂SO₄, filtered and concentrated in vacuum. The residue was purified by flash chromatography (ISCO®; 4 g SepaFlash® Silica Flash Column, petroleum ether/EtOAc with EtOAc from 0˜30%, flow rate=5 mL/min, 254 nm) to afford the title compound as a yellow solid (61.5 mg, 60.0%) ¹H NMR (400 MHz, DMSO-d₆) δ ppm 0.66-0.85 (m, 4H), 2.90 (br s, 1H), 3.18 (s, 3H), 3.73-3.75 (m, 2H), 4.54 (t, J=5.3 Hz, 2H), 7.35-7.41 (m, 2H), 7.98 (br s, 1H), 8.07 (br s, 1H), 8.43 (br s, 1H), 8.47 (s, 1H), 10.56 (br s, 1H); LCMS (ESI) m/z 393.2 [M+H]⁺.

### Example 134

### Synthesis of N⁴-cyclopropyl-N2-[1-(2,2-difluoroethyl)indazol-4-yl]-5-(trifluoromethyl) pyrimidine-2,4-diamine

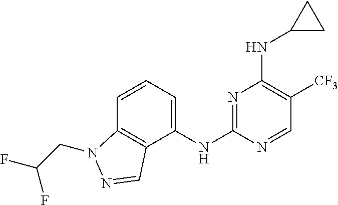 [SMILES: FC(F)Cn1ncc2c(Nc3ncc(C(F)(F)F)c(NC4CC4)n3)cccc21]

### Step 1

A mixture of 4-nitro-1H-indazole (20.0 g, 123 mmol, 10 eq) in DMF (400 mL), it was cooled to 0° C. under N₂ atmosphere, and then NaH (9.81 g, 245 mmol, 60 wt % in mineral oil, 2.0 eq.) was added. After 30 mins, 2-bromo-1,1-difluoro-ethane (35.5 g, 245 mmol, 2.0 eq.) was added, which was degassed and purged with N₂ for 3 times, and then the mixture was stirred at 20° C. for 16 hr tinder N₂ atmosphere. The solution was added H₂O (1500 mL), extracted with EtOAc (1000 mL×2), washed with LiCl/H₂O (800 mL×2), brine (800 mL 2). The organic layers were concentrated under reduced pressure. The crude was purified by flash chromatography (ISCO®; 220 g SepaFlash® Silica Flash Column, petroleum ether/EtOAc with EtOAc from 0˜50%, flow rate=100 mL/min, 254 nm) to give 1-(2,2-difluoroethyl)-4-nitro-indazole (Intermediate 134-a) as a yellow oil (14 g, 50.3%) with one regional isomers. LCMS (ESI) m/z 228.1 [M+H]⁺.

### Step 2

To a solution of 1-(2,2-difluoroethyl)-4-nitro-indazole (Intermediate 134-a) (9.50 g, 41.8 mmol, 1.0 eq.) in MeOH (100 mL) was added Pd/C (4.43 g, 4.18 mmol, 10 wt % Pd with 50 wt % water, 0.1 eq.) under N₂ atmosphere. The suspension was degassed and purged with H₂ (15 psi) for 3 times. The mixture was stirred under H₂ (15 psi) at 25° C. for 16 hr. The solution was filtered and concentrated under reduced pressure. The residue was purified by flash chromatography (ISCO®; 120 g SepaFlash® Silica Flash Column, petroleum ether/EtOAc with EtOAc from 0˜50%, flow rate=80 mL/min, 254 nm) to give 1-(2,2-difluoroethyl)indazol-4-amine (Intermediate 134-b) as a yellow solid (4.2 g, 50.9%). ¹H NMR (400 MHz, DMSO-d₆) δ ppm 4.78 (td, J=15.1, 3.8 Hz, 2H), 5.71-6.01 (m, 2H), 6.17-6.57 (m, 2H), 6.78 (d, J=8.2 Hz, 1H), 7.10 (t, J=7.8 Hz, 1H), 8.23 (s, 1H).

### Step 3

A mixture of 1-(2,2-difluoroethyl)indazol-4-amine (Intermediate 134-b) (100 μg, 0.507 mmol, 1.0 eq.), 2-chloro-N-cyclopropyl-5-(trifluoromethyl)pyrimidin-4-amine (Intermediate 68-a) (121 mg, 0.507 mmol, 1.0 eq.), 1N HCl/H₂O (50.7 μL, 50.7 μmol, 0.1 eq.) in i-PrOH (5 mL) was degassed and purged with N₂ for 3 times, and then the mixture was stirred at 80° C. for 1 hr under N₂ atmosphere. It was cooled 0° C. and lot of solid was precipitated. The mixture was filtered and triturated with EtOAc (10 mL). The residue was lyophilized to provide the title compound as a white solid (143 mg, 64.8%). ¹H NMR (400 MHz, DMSO-d₆) δ ppm 0.70-0.82 (m, 4H), 2.80-2.97 (m, 1H), 4.92 (td, J=15.1, 3.4 Hz, 2H), 6.24-6.61 (m, 1H), 7.37-7.47 (m, 2H), 7.88 (br s, 1H), 8.14 (br s, 1H), 8.42 (s, 1H), 8.57 (s, 1H), 10.56 (br s, 1H); LCMS (ESI) m/z 399.1 [M+H]⁺.

### Example 135

### Synthesis of N⁴-cyclopropyl-N²-(1-isopropylsulfonylindazol-4-yl)-5-(trifluoromethyl) pyrimidine-2,4-diamine

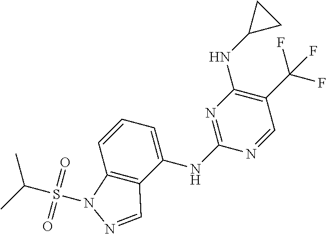 [SMILES: CC(C)S(=O)(=O)n1ncc2c(Nc3ncc(C(F)(F)F)c(NC4CC4)n3)cccc21]

### Step 1

A mixture of 4-nitro-2H-indazole (10.0 g, 61.3 mmol, 1.0 eq.), propane-2-sulfonyl chloride (17.5 g, 123 mmol, 2.0 eq.), NaH (4.90 g, 123 mmol, 2.0 eq., 60% purity in mineral oil) in DMF (150 mL) was degassed and purged with N₂ for 3 times, and then the mixture was stirred at 0-25° C. for 3 hr under N₂ atmosphere. The reaction mixture was quenched by addition H₂O 20 mL at 25° C. and extracted with EA mL (15 mL×3). The combined organic layers were washed with LiCl (15 mL×2), dried over and concentrated under reduced pressure. The residue was purified by flash silica gel chromatography (ISCO®); 220 g SepaFlash® Silica Flash Column, petroleum ether/EtOAc with EtOAc from 0˜40%, flow rate=100 mL/min, 254 nm) to give 1-isopropylsulfonylindazol-4-amine (Intermediate 135-a) as an orange solid (9.6 g, 65.5%). ¹H NMR (400 MHz, DMSO-d₆) δ ppm 1.23 (d, J=6.72 Hz, 6H), 3.94-4.08 (m, 1H), 7.85-7.92 (m, 1H), 8.33-8.49 (m, 2H), 8.98-9.01 (m, 1H); LCMS (ESI) m/z 270.0 [M+H]⁺.

### Step 2

A mixture of 1-isopropylsulfonyl-4-nitro-indazole (Intermediate 135-a) (9.60 g, 35.7 mmol, 1.0 eq.), NH₄Cl (19.1 g, 356 mmol, 10.0 eq.), Fe (20.0 g, 356 mmol, 10.0 eq.) in EtOH/H₂O (30 ml) was degassed and purged with N₂ for 3 times, and then the mixture was stirred at 50° C. for 2 hr under N₂ atmosphere. Filtered and extracted with EA (15 mL×3). The combined organic layers were washed with Na₂SO₄ and concentrated under reduced pressure. The residue was purified by flash silica gel chromatography (ISCO®; 220 g SepaFlash® Silica Flash Column, petroleum ether/EtOAc with EtOAc from 0˜40%, flow rate=80 mL/min, 254 nm) to give compound 1-isopropylsulfonylindazol-4-amine (Intermediate 135-b) as a yellow solid (6.5 g, 76.2%). ¹H NMR (500 MHz, DMSO-d₆) δ ppm 1.15 (d, J=6.9 Hz, 6H), 3.67-3.79 (m, 1H), 6.11-6.25 (m, 2H), 6.38-6.47 (m, 1H), 6.99-7.07 (m, 1H), 7.18-7.25 (m, 1H), 8.54-8.66 (m, 1H); LCMS (ESI) m/z 240.0 [M+H]⁺.

### Step 3

A mixture of 1-isopropylsulfonylimidazol-4-amine (Intermediate 135-b) (100 mg, 0.418 mmol, 1.0 eq.), 2-chloro-N-cyclopropyl-5-(trifluoromethyl)pyrimidin-4-amine (Intermediate 68-a) (99.3 mg, 0.418 mmol, 1.0 eq.), 1N HCl/H₂O (42.0 uL, 0.0418 nmol, 0.1 eq.) in IPA (2 mL) was degassed and purged with N₂ for 3 times, and then the mixture was stirred at 80° C. for 1 hr under N₂ atmosphere. Filtered and concentrated under reduced pressure. The residue was triturated with EA (5 mL) at 25° C. for 1 hr to provide the title compound as a white solid (68.5 mg, 37.2%). ¹H NMR (400 MHz, DMSO-d₆) δ ppm 0.69-0.82 (m, 4H), 1.18 (d, J=6.8 Hz, 6H), 2.73-2.92 (m, 1H), 3.83 (br s, 1H), 7.58-7.69 (m, 2H), 7.84-7.98 (m, 1H), 8.25-8.36 (m, 1H), 8.43-8.47 (m, 1H), 8.97-9.04 (m, 1H), 10.75-10.85 (m, 1H); LCMS (ESI) m/z 441.2 [M+H]⁺.

### Example 136

### Synthesis of N⁴-cyclopropyl-N²-(1-((2-methoxyethyl)sulfonyl)-1H-indazol-4-yl)-5-(trifluoromethyl)pyrimidine-2,4-diamine

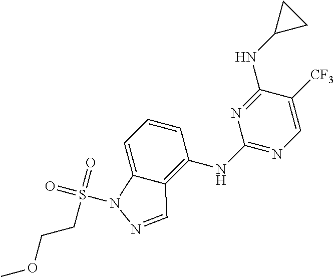 [SMILES: COCCS(=O)(=O)n1ncc2c(Nc3ncc(C(F)(F)F)c(NC4CC4)n3)cccc21]

### Step 1

A mixture of 4-nitro-1H-indazole (1.00 g, 6.13 mmol, 1.0 eq.), 2-methoxyethanesulfonyl chloride (972 mg, 6.13 mmol, 1.0 eq.), TEA (1.86 g, 18.4 mmol, 3.0 eq.) in DCM (10 mL) was degassed and purged with N₂ for 3 times, and then the mixture was stirred at 25° C. for 2 hr under N₂ atmosphere. The reaction mixture was added H₂O (10 mL) and extracted with DCM (10 mL×3). The combined organic layers were dried with anhydrous Na₂SO₄, filtered and concentrated under reduced pressure. The residue was purified by flash silica gel chromatography (ISCO®; 40 g SepaFlash® Silica Flash Column, dichloromethane/methanol with methanol from 0˜5%, flow rate=30 mL/min, 254 nm) to provide compound 1-((2-methoxyethyl)sulfonyl)-4-nitro-1H-indazole (Intermediate 136-a) as a yellow solid (325 mg, 18.2%). ¹H NR (400 MHz, DMSO-d₆) δ ppm 2.76 (s, 3H), 3.56-3.61 (m, 2H), 4.01 (s, 2H), 7.85 (br d, J=1.5 Hz, 1H), 8.35-8.38 (m, 1H), 8.41-8.46 (m, 1H), 8.96-8.99 (m, 1H); LCMS (ESI) m/z 285.7 [M+H]⁺.

### Step 2

To a solution of 1-((2-methoxyethyl)sulfonyl)-4-nitro-1H-indazole (Intermediate 136-a) (1.80 g, 6.31 mmol, 1.0 eq.) and Pd/C (1.0 g, 10 wt % Pd with 50 wt % water) in MeOH (20 ml) was degassed and purged with H₂ (15 psi) for 3 times, and then the mixture was stirred at 25° C. for 2 hr under 12 (15 psi) atmosphere. The reaction mixture was filtered and concentrated under reduced pressure. The residue was purified by flash silica gel chromatography (ISCO®; 80 g SepaFlash® Silica Flash Column, petroleum ether/EtOAc with EtOAc from 0˜60%, flow rate=60 mL/min, 254 nm) to provide compound 1-((2-methoxyethyl)sulfonyl)-1H-indazol-4-amine (Intermediate 136-b) as a yellow oil (1.41 g, 87.4%). ¹H NMR (400 MHz, DMSO-d₆) δ ppm 2.94 (s, 3H), 3.48-3.58 (m, 2H), 3.74 (t, J=5.52 Hz, 2H), 6.16 (s, 2H), 6.41 (d, J=7.8 Hz, 1H), 7.02 (d, J=8.3 Hz, 1H), 7.22 (t, J=7.9 Hz, 1H), 8.57 (s, 1H); LCMS (ESI) m/z 256.0 [M+H]⁺.

### Step 3

A mixture of 1-((2-methoxyethyl)sulfonyl)-1H-indazol-4-amine (Intermediate 136-b) (100 mg, 0.390 mmol, 1.0 eq.), 2-chloro-N-cyclopropyl-5-(trifluoromethyl)pyrimidin-4-amine (Intermediate 68-a) (93.1 mg, 0.390 mmol, 1.0 eq.) and 1N HCl/H₂O (0.59 mL, 0.585 mmol, 1.5 eq.) in IPA (1 mL) was stirred at 80° C. for 4 hr under N2 atmosphere. The reaction mixture was added Na₂CO₃/H₂O (5 mL) and extracted with EtOAc (5 mL×3). The combined organic layers were dried with anhydrous Na₂SO₄, filtered and concentrated under reduced pressure to give a residue. The residue was purified by flash silica gel chromatography (ISCO®; 4 g SepaFlash® Silica Flash Column, petroleum ether/EtOAc with EtOAc from 0˜50%, flow rate=20 mL/min, 254 nm) to provide the title compound as a white solid (140.4 mg, 78.5%). ¹H NMR (400 MHz, DMSO-d₆) δ ppm 0.66-0.72 (m, 2H), 0.74-0.82 (m, 2H), 2.81-2.87 (m, 1H), 2.87-2.92 (m, 3H), 3.53-3.59 (m, 3H), 3.80-3.90 (m, 2H), 7.22-7.36 (m, 1H), 7.50-7.61 (m, 2H), 8.25-8.31 (m, 1H), 8.34-8.44 (m, 1H), 8.94 (s, 1H), 10.08-10.17 (m, 1H); ¹⁹F NMR (377 MHz, DMSO-d₆) δ ppm −60.197; LCMS (ESI) m/z 457.0 [M+H]⁺.

### Example 137

### N²-(1-(cyclopropylsulfonyl)-1H-indazol-4-yl)-N⁴-(2-fluoroethyl)-5-(trifluoromethyl) pyrimidine-2,4-diamine

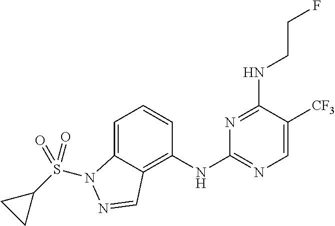 [SMILES: O=S(=O)(C1CC1)n1ncc2c(Nc3ncc(C(F)(F)F)c(NCCF)n3)cccc21]

### Step 1: 1-(cyclopropylsulfonyl)-4-nitro-1H-indazole

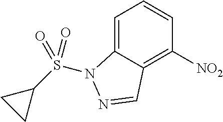 [SMILES: O=[N+]([O-])c1cccc2c1cnn2S(=O)(=O)C1CC1]

To a mixture of 4-nitro-1H-indazole (5.00 g, 30.7 mmol), cyclopropanesulfonyl chloride (5.21 g, 37.1 mmol) in THF (50 mL) was added NaH (1.47 g, 36.8 mmol) at 0° C. under N₂ atmosphere, then the mixture was allowed to warm up to 20° C. and stirred for 2 hours. The reaction mixture was quenched by H₂O (50 mL) at 25° C., and extracted with EtOAc (100 mL×3). The combined organic layers were washed with brine (100 mL×3), dried over anhydrous Na₂SO₄, filtered and concentrated under reduced pressure. The residue was purified by flash chromatography (ISCO®; 80 g SepaFlash® Silica Flash Column, petroleum ether/EtOAc with EtOAc from 0˜20%, flow rate: 40 mL/min) to provide 1-cyclopropylsulfonyl-4-nitro-indazole (Intermediate 137-a) as a white solid (3.50 g, 42.7% yield). ¹H NMR (400 MHz, CDCl₃) δ ppm 1.14-1.20 (m, 2H), 1.52-1.56 (m, 21H), 2.82 (tt, J=8.0, 4.7 Hz, 1H), 7.67-7.74 (m, 1H), 8.29-8.33 (m, 1H), 8.47 (d, J=8.5 Hz, 1H), 8.94 (d, J=0.8 Hz, 1H); LCMS (ESI) m/z 267.9 [M+H]⁺.

### Step 2: 1-(cyclopropylsulfonyl)-1H-indazol-4-amine

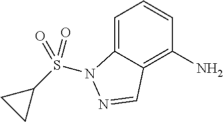 [SMILES: Nc1cccc2c1cnn2S(=O)(=O)C1CC1]

A mixture of 1-cyclopropylsulfonyl-4-nitro-indazole (Intermediate 137-a) (3.50 g, 13.1 mmol), Fe powder (3.66 g, 65.5 mmol) and NH₄C (350 g, 65.5 mmol) in a mixed solvent of EtOH (50 mL) and H₂O (50 mL) was stirred at 80° C. for 1 hour under N₂ atmosphere. The reaction mixture was quenched by H₂O (50 mL) at 25° C. and extracted with EtOAc (100 mL×3). The combined organic layers were washed with brine (100 mL×3), dried over anhydrous Na₂SO₄, filtered and concentrated under reduced pressure. The residue was purified by flash chromatography (ISCO®; 40 g SepaFlash® Silica Flash Column, petroleum ether/EtOAc with EtOAc from 0˜40%, flow rate: 40 mL/min) to provide 1-cyclopropylsulfonylindazol-4-amine (Intermediate 137-b) as a white solid (3.00 g 96.6% yield). H NMR (400 MHz, DMSO-d₆) δ ppm 1.15-1.21 (m, 4H), 2.93 (tt, J=7.9, 4.6 Hz, 1H), 6.19 (s, 2H), 6.43 (d, J=7.6 Hz, 1H), 7.03 (d, J=8.3 Hz, 1H), 7.23 (t, J=7.9 Hz, 1H), 8.60 (s, 1H); LCMS (ESI) m/z 238.0 [M+H]⁺.

### Step 3: N-(4-chloro-5-(trifluoromethyl)pyrimidin-2-yl)-1-(cyclopropylsulfonyl)-1H-indazol-4-amine

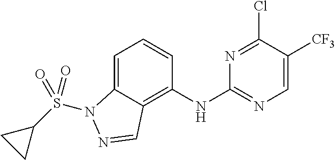 [SMILES: O=S(=O)(C1CC1)n1ncc2c(Nc3ncc(C(F)(F)F)c(Cl)n3)cccc21]

To a solution of 2,4-dichloro-5-(trifluoromethyl)pyrimidine (Intermediate 137-b) (185 mg, 0.853 mmol) in t-BuOH/DCE (1:1, 20 mL) was added dichlorozine (1 M, 1 mL) at 0° C. and the reaction was stirred for 30 minutes. Then the solution of 1-cyclopropylsulfonylindazol-4-amine (200 mg, 0.843 mmol) in t-BuOH/DCE (1:1, 10 mL) was added over 15 minutes at 0° C., followed by the addition of N,N-diethylethanamine (94.5 mg, 130 mL). The reaction mixture was allowed to warm up to 25° C. and stirred for 12 hours. The reaction mixture was quenched by H₂O (50 mL) at 25° C., and extracted with EtOAc (100 mL×3). The combined organic layers were washed with brine (100 mL×3), dried over anhydrous Na₂SO₄, filtered and concentrated under reduced pressure. The residue was purified by flash chromatography (ISCO®; 40 g SepaFlash® Silica Flash Column, petroleum ether/EtOAc with EtOAc from 0˜40%, flow rate: 40 mL/min), to provide N-[4-chloro-5-(trifluoromethyl)pyrimidin-2-yl]-1-cyclopropylsulfonyl-indazol-4-amine (Intermediate 137-c) as a white solid (300 mg, 85.2% yield). ¹H NMR (400 MHz, CDCl₃) δ ppm 1.06-1.15 (m, 2H), 1.48-1.54 (m, 2H), 2.78 (tt, J=8.0, 4.6 Hz, 1H), 7.54-7.62 (m, 1H), 7.91 (dd, J=15.9, 8.2 Hz, 3H), 8.34 (s, 1H), 8.64 (s, 1H); LCMS (ESI) m/z 418.1 [M+H]⁺.

### Step 4: N²-(1-(cyclopropylsulfonyl)-1H-indazol-4-yl)-N⁴-(2-fluoroethyl)-5-(trifluoromethyl) pyrimidine-2,4-diamine

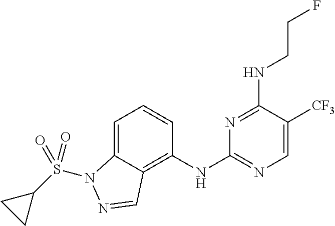 [SMILES: O=S(=O)(C1CC1)n1ncc2c(Nc3ncc(C(F)(F)F)c(NCCF)n3)cccc21]

A mixture of N-[4-chloro-5-(trifluoromethyl)pyrimidin-2-yl]-1-cyclopropylsulfonyl-indazol-4-amine (Intermediate 137-c) (150 mg, 0.359 mmol), 2-fluoroethanamine hydrochloride (75.0 mg, 1.05 mmol) and TEA (109 mg, 150 mL) in t-BuOH (10 ml) was stirred at 40° C. for 12 hours under N₂ atmosphere. The residue was filtered and the filtrate was concentrated under reduced pressure. The residue was purified by preparative HPLC (Instrument: Gilson GX-281 Liquid Handler, Gilson 322 Pump, Gilson 156 UV Detector; Column: Welch Xtimate C18 150×25 mm×5 μm Mobile phase A: H₂O with 0.05% FA (v %); Mobile phase B: ACN; Gradient: B from 70% to 100% in 7.8 min, hold 100% B for 2 min; Flow Rate: 25 mL/min; Column Temperature: 30° C.; Wavelength: 220 nm) to provide the title compound as a white solid (35.0 mg, 21.9% yield). ¹H NMR (400 MHz, DMSO-d₆) δ ppm 1.07-1.14 (m, 2H), 1.22-1.25 (m, 2H), 3.02-3.10 (m, 1H), 3.69-3.81 (m, 2H), 4.51 (t, J=5.3 Hz, 1H), 4.63 (t, J=5.3 Hz, 1H), 7.35 (br t, J=5.5 Hz, 1H), 7.52-7.65 (m, 2H), 7.95 (d, J=7.6 Hz, 1H), 8.30 (s, 1H), 8.83 (s, 1H), 10.11 (s, 1H); ¹⁹F NMR (377 MHz, DMSO-d₆) δ ppm −60.38, −222.57; LCMS (ESI) m/z 445.0 [M+H]⁺.

### Example 138

### N²-(1-(cyclopropylsulfonyl)-1H-indazol-4-yl)-N⁴-(2-(methylsulfonyl)ethyl)-5-(trifluoromethyl)pyrimidine-2,4-diamine

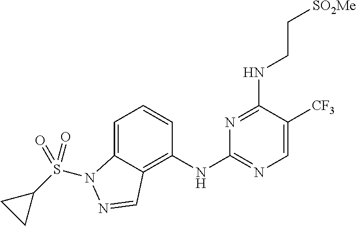 [SMILES: CS(=O)(=O)CCNc1nc(Nc2cccc3c2cnn3S(=O)(=O)C2CC2)ncc1C(F)(F)F]

A mixture of N-[4-chloro-5-(trifluoromethyl)pyrimidin-2-yl]-1-cyclopropylsulfonyl-indazol-4-amine (Intermediate 137-c) (100 mg, 0.240 mmol), 2-methylsulfonylethanamine (150 mg, 0.940 mmol, HCl) and TEA (72.7 mg, 0,718 mmol) in t-BuOH (10 mL) was stirred at 20° C. for 12 hours tinder N₂ atmosphere. The residue was filtered and the filtrate was concentrated under reduced pressure. The residue was purified by preparative HPLC (Instrument: Gilson GX-281 Liquid Handler, Gilson 322 Pump, Gilson 156 UV Detector; Column: Welch Xtimate C18 150×25 mm×5 μm; Mobile phase A: H₂O with 0.05% NH₃—H₂O (v %); Mobile phase B: ACN; Gradient: B from 70% to 100% in 7.8 min, hold 100% B for 2 min; Flow Rate: 25 mL/min; Column Temperature: 30° C.; Wavelength: 220 nm) to provide the title compound as a white solid (50 mg, 41.4% yield). ¹H NMR (400 MHz, DMSO-d₆) δ ppm 1.07-1.13 (m, 2H), 1.22-1.27 (m, 2H), 2.99 (s, 3H), 3.02-3.09 (m, 1H), 3.43 (br t, J=6.8 Hz, 2H), 3.88 (q, J=6.4 Hz, 2H), 7.34-7.42 (m, 1H), 7.55-7.65 (m, 2H), 8.05 (d, J=7.3 Hz, 1H), 8.32 (s, 1H), 8.86 (s, 1H), 10.14 (s, 1H); ¹⁹F NMR (377 MHz, DMSO-d₆) δ ppm −60.29; LCMS (ESI) m/z 505.0 [M+H]⁺.

### Example 139

### N⁴-cyclopropyl-N²-(1-(2-(methylsulfonyl)ethyl)-1H-indazol-4-yl)-5-(trifluoromethyl)pyrimidine-2,4-diamine

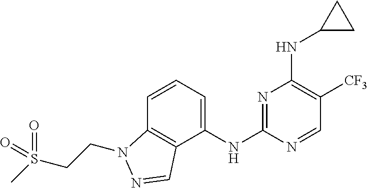 [SMILES: CS(=O)(=O)CCn1ncc2c(Nc3ncc(C(F)(F)F)c(NC4CC4)n3)cccc21]

### Step 1: 1-(2-(methylsulfonyl)ethyl)-4-nitro-1H-indazole

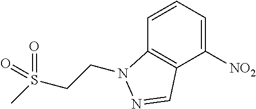 [SMILES: CS(=O)(=O)CCn1ncc2c([N+](=O)[O-])cccc21]

To a solution of 4-nitro-1H-indazole (200 mg, 1.23 mmol, 1 eq) in DCM (1 mL) was added TEA (372 mg, 3.68 mmol, 3 eq) and 1-bromo-2-methylsulfonyl-ethane (229 mg, 1.23 mmol, 1 eq) at 0° C. The mixture was degassed and purged with N₂ for three times, then stirred at 50° C. for 16 hrs under N₂ atmosphere. The residue was diluted with H₂O (10 mL) and extracted with DCM (15 mL×3). The combined organic layers were washed with brine (10 mL), dried over Na₂SO₄, filtered and concentrated under reduced pressure to give a residue, which was purified by prep-HPLC (column: Welch Xtimate C18 100×40 mm×3 μm; mobile phase: [water (HCl)-ACN]; B %: 25%-55%, 8 min as additive) to give 1-(2-methylsulfonylethyl)-4-nitro-indazole (Intermediate 139-a) as a white solid (90 mg, 23.45%). LCMS (ESI) m/z 269.9 [M+H]⁺.

### Step 2: 1-(2-(methylsulfonyl)ethyl)-1H-imidazol-4-amine

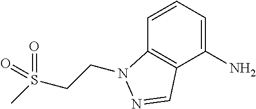 [SMILES: CS(=O)(=O)CCn1ncc2c(N)cccc21]

To a solution of I-(2-methylsulfonylethyl)-4-nitro-indazole (Intermediate 139-a) (90 mg, 0.334 mmol, 1 eq) in MeOH (2 mL) was added Pd/C (35.5 mg, 0.033 mmol, 10% purity, 01 eq). The mixture was degassed and purged with H₂ for three times, then stirred at 25° C. for 2 hrs under H₂ atmosphere. The mixture was filtered and the filtrate was concentrated under reduced pressure to give 1-(2-methylsulfonylethyl)indazol-4-amine (Intermediate 139-b) as a yellow oil (80 mg, crude), which was used directly without further purification. LCMS (ESI) m/z 239.9 [M+H]⁺.

### Step 3: N⁴-cyclopropyl-N²-(1-(2-(methylsulfonyl)ethyl)-1H-indazol-4-yl)-5-(trifluoromethyl)pyrimidine-2,4-diamine

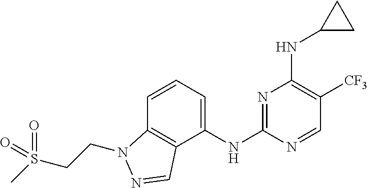 [SMILES: CS(=O)(=O)CCn1ncc2c(Nc3ncc(C(F)(F)F)c(NC4CC4)n3)cccc21]

To a solution of 1-(2-methylsulfonylethyl)indazol-4-amine (Intermediate 139-b) (70 mg, 0.292 mmol, 1 eq) in i-PrOH (1 ml) was added 2-chloro-N-cyclopropyl-5-(trifluoromethyl)pyrimidin-4-amine (Intermediate 68-a) (69.51 mg, 0.292 mmol, 1 eq) and HC (1 M, 0.43 mL, 1.5 eq). The mixture was degassed and purged with N₂ for three times, then stirred at 80° C. for 4 hrs under N₂ atmosphere. The mixture was basified with NaHCO₃(aq) to pH=6-7 at 0° C. and extracted with EtOAc (10 mL×3). The combined organic layers were washed with brine (10 mL), dried by Na₂SO₄, filtered and concentrated under reduced pressure to give a residue. The residue was purified by flash chromatography (ISCO® 4 g SepaFlash® Silica Flash Column, petroleum ether/EtOAc with EtOAc from 0˜80%, 30 mL/min, 254 mn/I₂/KMnO₄/PMA) to provide the title compound as a white solid (50.13 mg, 37.7%). ¹H NMR (400 MHz, DMSO-d₆) δ ppm 0.67-0.73 (m, 2H), 0.77-0.83 (m, 2H), 2.89 (s, 3H), 3.30 (s, 1H), 3.73 (t, J=6.78 Hz, 2H), 4.78 (t, J=6.78 Hz, 2H), 7.24 (br s, 1H), 7.28-7.33 (m, 1H), 7.34-7.40 (n, 1H), 8.22-8.30 (m, 2H), 8.56 (s, 1H), 9.90 (s, 1H). ¹⁹F NMR (376 MHz, DMSO-d₆) δ ppm −60.04 (br s, 3F); LCMS (ESI) m/z 441.0 [M+H]⁺.

### Example 140

### N⁴-isopropyl-N²-[1-(2-methoxyethyl)indazol-4-yl]-5-(trifluoromethyl)pyrimidine-2,4-diamine

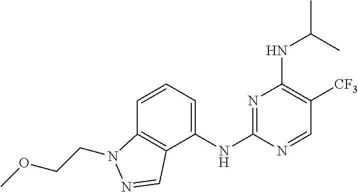 [SMILES: COCCn1ncc2c(Nc3ncc(C(F)(F)F)c(NC(C)C)n3)cccc21]

### Step 1: 2-chloro-N-isopropyl-5-(trifluoromethyl)pyrimidin-4-amine

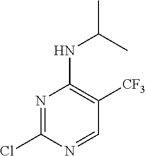

A mixture of propan-2-amine (2.72 g, 46.09 mmol), 2,4-dichloro-5-(trifluoromethyl)pyrimidine (10 g, 46.09 mmol), K₂CO₃ (6.37 g, 46.09 mmol) in DMF (50 mL) was degassed and purged with N₂ for 3 times, and then stirred at 20° C. for 12 hrs under N₂ atmosphere. The resulting mixture was extracted with EtOAc (30 mL×3). The combined organic layers were washed with saturated brine (30 mL×3), dried over anhydrous Na₂SO₄, filtered and concentrated under reduced pressure. The residue was purified by preparative HPLC (Instrument: Gilson GX-281 Liquid Handler, Gilson 322 Pump, Gilson 156 UV Detector; Column: Welch Xtimate C18 150×25 mm×5 μm; Mobile phase A: H₂O with 0.05% NH₃—H₂O (v %); Mobile phase B: ACN; Gradient: B from 70% to 100% in 7.8 min, hold 100% B for 2 min; Flow Rate: 25 mL/min; Column Temperature: 30° C.; Wavelength: 220 nm) to provide 4-chloro-N-isopropyl-5-(trifluoromethyl)pyrimidin-2-amine as a white solid (4.0 g, 34.41% yield, 95% purity, faster eluting isomer) and 2-chloro-N-isopropyl-5-(trifluoromethyl)pyrimidin-4-amine (Intermediate 140-a) as a colorless oil (1.42 g, 5.87 mmol, 12.73% yield, 99% purity, slower eluting isomer). ¹H NMR (400 MHz, CD₃OD) δ ppm 1.27-1.29 (d, J=6.40 Hz, 6H), 4.42-4.56 (m, 1H), 4.61 (s, 1H), 8.25 (s, 1H); LCMS (ESI) m/z 239.9 [M+H]⁺.

### Step 2: N⁴-isopropyl-N²-[1-(2-methoxyethyl)indazol-4-yl]-5-(trifluoromethyl)pyrimidine-2,4-diamine

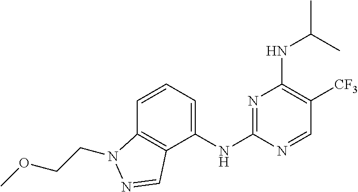 [SMILES: COCCn1ncc2c(Nc3ncc(C(F)(F)F)c(NC(C)C)n3)cccc21]

A mixture of 1-(2-methoxyethyl)indazol-4-amine (Intermediate 134-b) (100 mg, 523 μmol), 2-chloro-N-isopropyl-5-(trifluoromethyl)pyrimidin-4-amine (Intermediate 140-a) (125 mg, 523 mmol) and HC (1 M, 52.3 mL) in IPA (3 mL) was stirred at 80° C. for 1 hr under N₂ atmosphere. The reaction was purified by prep-HPLC (Boston Green ODS 150×30 mm×5 μm; mobile phase: [water (HCl)-ACN]; B %: 32%-72%, 9 min) to provide the title compound as a white solid (35 mg, 17.0% yield), ¹H NMR (400 MHz, CD₃OD) δ ppm 1.25 (br d, J=6.53 Hz, 6H), 3.27 (s, 3H), 3.85 (t, J=5.14 Hz, 2H), 4.42-4.56 (m, 1H), 4.61-4.67 (m, 2H), 7.37-7.44 (m, 1H), 7.47-7.55 (m, 1H), 763 (br d, J=8.53 Hz, 1H) 8.15-8.25 (m, 2H); LCMS (ESI) m/z 395.2 [M+H]⁺.

### Example 141

### 2-[[2-[[1-(2,2-difluoroethyl)indazol-4-yl]amino]-5-(trifluoromethyl)pyrimidin-4-yl]amino]ethanol

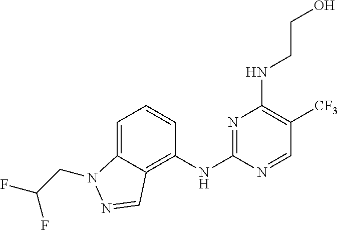 [SMILES: OCCNc1nc(Nc2cccc3c2cnn3CC(F)F)ncc1C(F)(F)F]

### Step 1: N-[4-chloro-5-(trifluoromethyl) pyrimidin-2-yl]-1-(2,2-difluoroethyl)indazol-4-amine

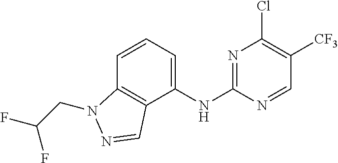 [SMILES: FC(F)Cn1ncc2c(Nc3ncc(C(F)(F)F)c(Cl)n3)cccc21]

A mixture of 1-(2,2-difluoroethyl)-1H-indazol-4-amine (Intermediate 94-b) (500 mg, 2.54 mmol), 2,4-dichloro-5-(trifluoromethyl)pyrimidine (551 mg, 2.54 mmol) and DIPEA (394 ng, 3.05 nmol) in t-BuOH (10 mL) was stirred at 80° C. for 0.5 hr under N₂ atmosphere. The resulting mixture was quenched with water (5 mL) and extracted with EtOAc (10 mL×3). The organic layer was washed with brine (10 mL) and concentrated in vacuo. The residue was purified by flash silica gel chromatography (ISCO®; 12 g SepaFlash® Silica Flash Column, Eluent of 0˜35% Ethyl acetate/Petroleum ether gradient @ 12 mL/min) to provide N-[4-chloro-5-(trifluoromethyl)pyrimidin-2-yl]-1-(2,2-difluoroethyl)indazol-4-amine (Intermediate 141-a) as a white solid (370 mg, 38.5% yield). ¹H NMR (400 MHz, DMSO-d₆) δ ppm 4.87-4.99 (m, 2H), 6.28-6.59 (m, 1H), 7.38-7.56 (m, 2H), 8.31 (s, 1H) 8.75 (s, 1H), 10.99 (s, 1H); LCMS (ESI) m/z 378.2 [M+H]⁺.

### Step 2: 2-[[2-[[1-(2,2-difluoroethyl)indazol-4-yl]amino]-5-(trifluoromethyl)pyrimidin-4-yl]amino]ethanol

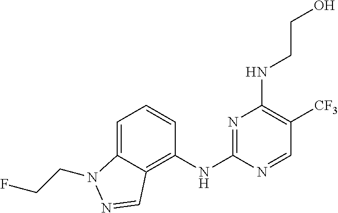 [SMILES: OCCNc1nc(Nc2cccc3c2cnn3CCF)ncc1C(F)(F)F]

A mixture of N-[4-chloro-5-(trifluoromethyl)pyrimidin-2-yl]-1-(2,2-difluoroethyl)indazol-4-amine (Intermediate 141-a) (100 mg, 265 μmol), 2-aminoethanol (16.2 mg, 265 μmol) and HCl (26.5 μmol, 94.6 μL, 1% purity) in IPA (2 mL) was stirred at 80° C. for 0.5 hr under N2 atmosphere. The reaction was purified by prep-HPLC (column: Boston Green ODS 150×30 mm×5 μm; mobile phase: [water (HCl)-ACN]; 13%: 10%-50%, 9 min) to provide the title compound as a white solid (29.1 mg, 27.3% yield). ¹H NMR (400 MHz, DMSO-d₆) δ ppm 3.55-3.58 (m, 4H), 4.86-5.04 (m, 2H), 6.19-6.67 (m, 1H), 7.38-7.54 (m, 2H), 7.72-7.83 (m, 1H), 8.10 (br s, 1H), 8.47-8.56 (m, 2H), 10.96 (br s, 1H); LCMS (ESI) m/z 403.2 [M+H]⁺.

### Example 142

### N²-[1-(2,2-difluoroethyl)indazol-4-yl]-N⁴-(2-fluoroethyl)-5-(trifluoromethyl) pyrimidine-2,4-diamine

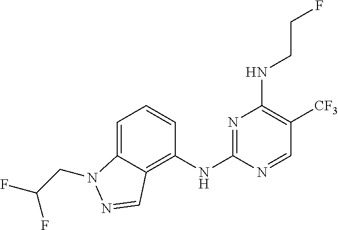 [SMILES: FCCNc1nc(Nc2cccc3c2cnn3CC(F)F)ncc1C(F)(F)F]

A mixture of N-[4-chloro-5-(trifluoromethyl)pyrimidin-2-yl]-1-(2,2-difluoroethyl)indazol-4-amine (Intermediate 141-a) (100 mg, 265 μmol), 2-fluoroethanamine hydrochloride (58.0 mg, 583 μmol) and DIEA (103 mg, 794 μmol) in t-BuOH (2 mL) was stirred at 80° C. for 0.5 hr under N₂ atmosphere. The residue was purified by prep-HPLC (column: Boston Prime C18 150×30 mm×5 μm; mobile phase. [water (ammonia hydroxide v/v)-ACN]; B %: 40%-70%, 8 min) to provide the title compound as a white solid (43.7 mg, 40.8% yield). ¹H NMR (500 MHz, DMSO-d₆) δ ppm 3.65-3.90 (m, 2H), 4.47-4.68 (m, 2H), 4.80-4.95 (m, 2H), 6.21-6.55 (m, 1H), 7.20-7.32 (m, 1H), 7.33-7.37 (m, 2H), 7.69 (s, 1H), 8.19 (s, 1H), 8.38 (s, 1H), 9.81 (s, 1H); LCMS (ESI) m/z 405.2 [M+H]⁺.

### Example 11

### N⁴-cyclopropyl-N²-(1-ethylsulfonylindazol-4-yl)-5-(trifluoromethyl)pyrimidine-2,4-diamine

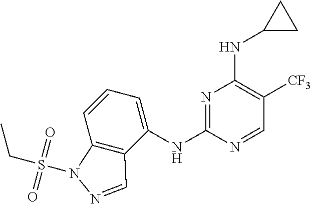 [SMILES: CCS(=O)(=O)n1ncc2c(Nc3ncc(C(F)(F)F)c(NC4CC4)n3)cccc21]

### Step 1: 1-ethylsulfonyl-4-nitro-indazole

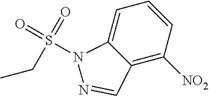 [SMILES: CCS(=O)(=O)n1ncc2c([N+](=O)[O-])cccc21]

A mixture of 4-nitro-1H-indazole (5 g, 30.65 mmol, 1 eq), ethanesulfonyl chloride (3.94 g, 30.65 mmol, 2.90 mL, 1 eq), TEA (9.30 g, 91.95 mmol, 3 eq) in DCM (100 mL) was degassed and purged with N₂ for 3 times, and then stirred at 0° C. for 2 hr under N₂ atmosphere. The residue was diluted with H₂O (100 mL) and extracted with DCM (150 mL×3). The combined organic layers were washed with brine (10 mL), dried over Na₂SO₄, filtered and concentrated under reduced pressure. The residue was purified by flash silica gel chromatography (ISCO®; 40 g SepaFlash® Silica Flash Column, Eluent of 0˜100% petroleum ether/EtOAc @ 40 mL/min) to give 1-ethylsulfonyl-4-nitro-indazole (Intermediate 143-a) as a yellow solid (13.5 g, 68.16%). LCMS (ESI) m/z 2559 [M+H]⁺.

### Step 2: 1-ethylsulfonylindazol-4-amine

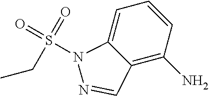

A mixture of 1-ethylsulfonyl-4-nitro-indazole (Intermediate 143-a) (4.5 g, 17.63 mmol, 1 eq), is Pd/C (1.88 g, 1.76 mmol, 10% purity, 0.1 eq) in MeOH (150 mL) was degassed and purged with H₂ for 3 times, and then stirred at 25° C. for 2 hrs under H₂ atmosphere. The resulting mixture was filtered and the filtrate was concentrated under reduced pressure to give 1-ethylsulfonylindazol-4-amine (Intermediate 143-b) as a black oil (10 g, 75.54%) LCMS (ESI) m/z 225.9 [M+H]⁺.

### Step 3: N⁴-cyclopropyl-N²-(1-ethylsulfonylindazol-4-yl)-5-(trifluoromethyl)pyrimidine-2,4-diamine

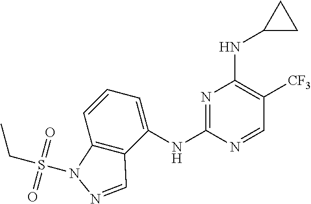 [SMILES: CCS(=O)(=O)n1ncc2c(Nc3ncc(C(F)(F)F)c(NC4CC4)n3)cccc21]

A mixture of 1-ethylsulfonylindazol-4-amine (Intermediate 143-b) (100 mg, 0.44 mmol, 1 eq), 2-chloro-N-cyclopropyl-5-(trifluoromethyl)pyrimidin-4-amine (Intermediate 68-a) (116 mg, 0.48 mmol, 1.1 eq) and HCl (1M, 0.66 mL, 1.5 eq) in IPA (I mL) was degassed and purged with N₂ for 3 times, and then stirred at 80° C. for 4 hr under N₂ atmosphere. The residue was diluted with NaHCO₃ (10 mL) and extracted with EtOAc (15 mL×3). The combined organic layers were washed with brine (10 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜25% petroleum ether/EtOAc @ 40 mL/min) to provide the title compound as a white solid (108.86 mg, 56.36%). ¹H NMR (500 MHz, CD₃OD) δ ppm 0.62-0.66 (m, 2H), 0.78-0.82 (m, 2H), 1.16 (t, J=7.40 Hz, 3H), 2.82 (tt, J=7.15, 3.76 Hz, 1H), 3.53 (q, J=7.38 Hz, 2H), 7.55 (t, J=8.16 Hz, 1H), 7.73 (d, J=8.39 Hz, 1H), 8.19 (s, 1H), 8.22 (d, J=7.78 Hz, 1H), 8.62 (s, 1H); ¹⁹F NMR (471 MHz, CD₃OD) δ ppm −63.5; LCMS (ESI) m/z 427.0 [M+H]⁺.

### Example 144

### 2-(4-((4-((2-fluoroethyl)amino)-5-(trifluoromethyl)pyrimidin-2-yl)amino)-1H-indazol-1-yl)-2-methylpropanenitrile

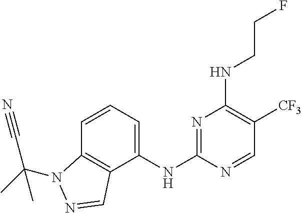 [SMILES: CC(C)(C#N)n1ncc2c(Nc3ncc(C(F)(F)F)c(NCCF)n3)cccc21]

### Step 1: Synthesis of 2-chloro-N-(2-fluoroethyl)-5-(trifluoromethyl)pyrimidin-4-amine

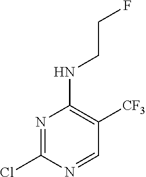 [SMILES: FCCNc1nc(Cl)ncc1C(F)(F)F]

A mixture of 2,4-dichloro-5-(trifluoromethyl)pyrimidine (2 g, 9.22 mmol, 1 eq), 2-fluoroethanamine (0.917 g, 9.22 mmol, 1 eq, HCl) and TEA (2.80 g, 27.6 mmol, 3 eq) in DCM (20 mL) was degassed and purged with N₂ for 3 times, and then stirred at 25° C. for 2 hr under N₂ atmosphere. The reaction mixture was concentrated under reduced pressure. The residue was quenched by H₂O (20 mL) and extracted with EtOAc (20 mL×3). The combined organic layers were washed with brine (40 mL), dried with anhydrous Na₂SO₄, filtered and concentrated under reduced pressure to give a residue. The residue was purified by flash silica gel chromatography (ISCO®; 20 g SepaFlash® Silica Flash Column, Eluent of 0-20% Ethyl acetate/Petroleum ethergradient @ 50 mL/mini) to provide 2-chloro-N-(2-fluoroethyl)-5-(trifluoromethyl)pyrimidin-4-amine (Intermediate 144-a) as a colorless oil (410 mg, 16.98%). ¹H NMR (400 MHz, DMSO-d6) δ ppm 3.65-3.79 (m, 2H), 4.48-4.67 (m, 1H), 4.49-4.66 (m, 1H), 8&06 (br s, 1H), 8.42 (s, 1H); LCMS (ESI) m/z 243.9 [M+H]⁺.

### Step 2: Synthesis of 2-(4-((4-((2-fluoroethyl)amino)-5-(trifluoromethyl)pyrimidin-2-yl)amino)-1H-indazol-1-yl)-2-methylpropanenitrile

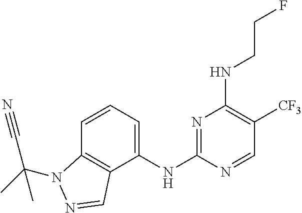 [SMILES: CC(C)(C#N)n1ncc2c(Nc3ncc(C(F)(F)F)c(NCCF)n3)cccc21]

A mixture of 2-(4-aminoindazol-1-yl)-2-methyl-propanenitrile (Intermediate 31-d) (62.50 mg, 0.250 mmol, 80% purity, 1 eq), 2-chloro-N-(2-fluoroethyl)-5-(trifluoromethyl)pyrimidin-4-amine (Intermediate 144-a) (65.40 mg, 0.250 mmol, 93% purity, 1 eq) and HCl (1 M, 0.375 mL, 1.5 eq) in IPA (0.5 mL) was degassed and purged with N₂ for 3 times, and then stirred at 80° C. for 4 hr under N₂ atmosphere. The reaction mixture was quenched by NaHCO₃ aq (5 mL) and extracted with EtOAc (5 mL×3). The combined organic layers were dried with anhydrous Na₂SO₄, filtered and concentrated under reduced pressure to give a residue. The residue was purified by flash silica gel chromatography (ISCO®; 12 g SepaFlash® Silica Flash Column, Eluent of 0˜30% Ethyl acetate/Petroleum ethergradient @ 30 mL/min) to provide the title compound as a yellow solid (58.60 mg, 56.46%). ¹H NMR (400 MHz, DMSO-d₆) δ ppm 2.09 (s, 6H), 3.67-3.86 (m, 2H), 4.44-4.69 (m, 2H), 7.30 (br t, J=4.89 Hz, 1H), 7.38-7.61 (n, 2H), 7.77-7.94 (m, 1H), 8.28 (s, 1H), 8.51 (s, 1H), 9.94 (s, 1H); ¹⁹F NMR (377 MHz, DMSO-d₆) δ ppm −60.261, −222.554; LCMS (ESI) m/z 408.0 [M+H]⁺.

### Example 15

### N²-(1-cyclopropylsulfonylindazol-4-yl)-N⁴-(2-methoxyethyl)-5-(trifluoromethyl) pyrimidine-2,4-diamine

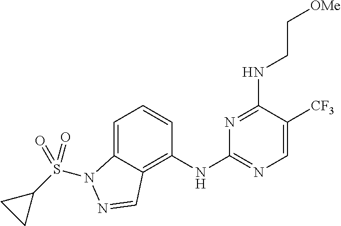 [SMILES: COCCNc1nc(Nc2cccc3c2cnn3S(=O)(=O)C2CC2)ncc1C(F)(F)F]

To a solution of N-[4-chloro-5-(trifluoromethyl)pyrimidin-2-yl]-1-cyclopropylsulfonyl-indazol-4-amine (Intermediate 137-c) (100 mg, 239 μmol) in THF/t-BuOH (1:1, 1 mL) was added 2-methoxyethanamine (500 mg, 6.66 mmol). The mixture was stirred at 20° C. for 2 hours, concentrated under reduced pressure and purified by preparative HPLC (Instrument: Gilson GX-281 Liquid Handler, Gilson 322 Pump, Gilson 156 UV Detector; Column: Welch Xtimate C18 150×25 mm 5 μm; Mobile phase A: H₂O with 0.05% NH₃—H₂O (v %); Mobile phase B: ACN, Gradient: B from 70% to 100% in 7.8 min, hold 100% B for 2 min; Flow Rate: 25 mL/min; Column Temperature: 30° C.; Wavelength: 220 nm) to provide the title compound as a white solid (55 mg, 49.9% yield). ¹H NMR (400 MHz, DMSO-d₆) δ ppm 1.06-1.11 (m, 2H), 1.20-1.26 (m, 2H), 2.97-3.10 (m, 1H), 3.22 (s, 3H), 3.46-3.49 (m, 2H), 3.56-3.60 (m, 2H), 7.14 (br t, J=5.25 Hz, 1H), 7.49-7.65 (m, 2H), 8.01 (d, J=7.75 Hz, 1H), 8.28 (s, 1H), 8.86 (s, 1H), 10.07 (s, 1H); ¹⁹F NMR (377 MHz, DMSO-d₆) δ ppm −60.40; LCMS (ESI) m/z 457.1 [M+H]⁺.

### Example 146

### 1-[4-[[4-(cyclopropylamino)-5-(trifluoromethyl)pyrimidin-2-yl]amino]indazol-1-yl]-2-methyl-propan-2-ol

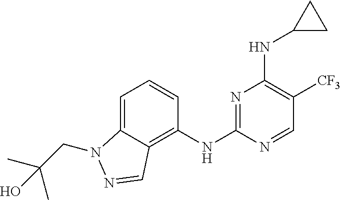 [SMILES: CC(C)(O)Cn1ncc2c(Nc3ncc(C(F)(F)F)c(NC4CC4)n3)cccc21]

A mixture of 1-(4-aminoindazol-1-yl)-2-methyl-propan-2-ol (Intermediate 97-b) (50 mg, 0.24 mmol, 1 eq), 2-chloro-N-cyclopropyl-5-(trifluoromethyl)pyrimidin-4-amine (Intermediate 68-a) (63.67 mg, 0.26 mmol, 1.1 eq) and HCl (1 M, 0.36 mL) in IPA (1 ml) was degassed and purged with N₂ for 3 times, and then stirred at 80° C. for 4 hours under N₂ atmosphere. The residue was diluted with NaHCO₃ (10 mL) and extracted with EtOAc (15 mL×3). The combined organic layers were washed with brine (10 mL), dried over Na₂SO₄, filtered and concentrated under reduced pressure to give a residue. The residue was purified by flash silica gel chromatography (ISCO®; 12 g SepaFlash®, Silica Flash Column, Eluent of 0˜20% petroleum ether/EtOAc @ 40 mL/Min) to give the title compound as a white solid (43.18 mg, 42.31%). ¹H NMR (400 MHz, CD₃OD) δ ppm 0.63-0.69 (m, 2H), 0.80-0.87 (m, 2H), 1.23 (s, 6H), 2.86 (tt, J=7.22, 3.70 Hz, 1H), 4.36 (s, 2H), 7.28-7.40 (m, 2H), 8.06 (d, J=7.28 Hz, 1H), 8.17 (s, 1H), 8.24 (s, 1H); ¹⁹F NMR (376 MHz, CD₃OD) δ ppm −63.38; LCMS (ESI) m/z 407.1 [M+H]⁺.

### Example 147

### 5-chloro-N²-(1-(2,2-difluoroethyl)-1H-indazol-4-yl)-N⁴-ethyl-7H-pyrrolo[2,3-d]pyrimidine-2,4-diamine

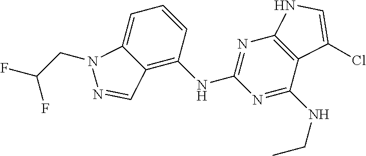 [SMILES: CCNc1nc(Nc2cccc3c2cnn3CC(F)F)nc2[nH]cc(Cl)c12]

### Step 1: 2-chloro-N-ethyl-7H-pyrrolo[2,3-d]pyrimidin-4-amine

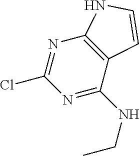

To a solution of 2,4-dichloro-1H-pyrrolo[2,3-d]pyrimidine (10 g, 53.2 mmol) in MeOH (100 mL) was added ethanamine (4.34 g, 53.2 mmol, HCl) and TEA (10.8 g, 106 mmol). The mixture was stirred at 80° C. for 3 hrs then filtered and the filtrate was concentrated under reduced pressure to give a crude product, which was purified by re-crystallization from MeOH (100 mL) at 25° C. to give 2-chloro-N-ethyl-7H-pyrrolo[2,3-d]pyrimidin-4-amine (Intermediate 147-a) as a white solid (6.5 g, 62.2% yield). ¹H NMR (400 MHz, DMSO-d₆) δ ppm 1.18-1.22 (m, 3H), 3.38-3.52 (M, 2H), 6.55 (dd, J=3.30, 1.96 Hz, 1H), 7.04-7.09 (m, 1H), 7.83 (s, 1H), 11.89 (m, 1H); LCMS (ESI) m/z 196.9 [M+H]⁺.

### Step 2: 2,5-dichloro-NV-ethyl-7H-pyrrolo[2,3-d]pyrimidin-4-amine

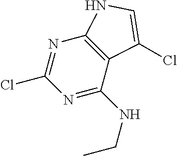

A mixture of 2-chloro-N-ethyl-7H-pyrrolo[2,3-d]pyrimidin-4-amine (Intermediate 147-a) (6.5 g, 33.1 mmol) and NCS (4.86 g, 36.4 mmol) in DMF (100 mL) was stirred at 25° C. for 3 hrs. The reaction mixture was filtered and the filtrate was concentrated under reduced pressure to give a crude product, which was purified by prep-HPLC (column: YMC-Triart Prep C18 150×40 mm×7 μm; mobile phase: [water(NH₃H₂O+NH₄HCO₃)-ACN]; 13%: 26%-66%, 9 min) to give 2,5-dichloro-N-ethyl-7H-pyrrolo[2,3-d]pyrimidin-4-amine (Intermediate 147-b) as a pink solid (2.6 g, 34.1% yield). ¹H NMR (400 MHz, DMSO-d₆) δ ppm 1.18 (t, J=7.07 Hz, 3H), 3.43-3.60 (m, 2H), 6.95-7.09 (m, 1H), 7.30 (s, 1H), 11.88 (s, 1H); LCMS (ESI) m/z 230.9 [M+H]⁺.

### Step 3: 5-chloro-N²-(1-(2,2-difluoroethyl)-1H-indazol-4-yl)-N4-ethyl-7H-pyrrolo[2,3-d]pyrimidine-2,4-diamine

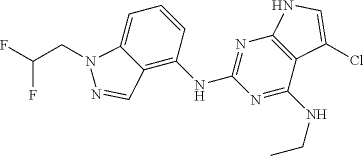 [SMILES: CCNc1nc(Nc2cccc3c2cnn3CC(F)F)nc2[nH]cc(Cl)c12]

To a solution of 1-(2,2-difluoroethyl)-1H-indazol-4-amine (Intermediate 94-b) (341 mg, 1.73 mmol) was added 2,5-dichloro-N-ethyl-7H-pyrrolo[2,3-d]pyrimidin-4-amine (Intermediate 147-b) (200 mg, 866 μmol) and TFA (160 μL, 2.16 mmol) in TEE (5 mL). The mixture was stirred at 90° C. for 16 hrs, then purified by prep-HPLC (column: Boston Green ODS 150×30 mm×5 μm; mobile phase: [water (HCl)-ACN]; B %: 27%-67%, 9 min and column: C18 (250×50 mm×10 μm); mobile phase: [water(NH₃H₂O+NH₄HCO₃)-ACN]; B %: 32%-72%, 9 min) to give the title compound as an orange solid (20 mg, 5.9% yield). ¹H NMR (400 MHz, DMSO-d₆) δ ppm 1.23 (br s, 3H), 3.58-3.65 (m, 2H), 4.69-5.01 (m, 2H), 6.22-6.60 (m, 2H), 6.91-7.09 (m, 1H), 7.13-7.43 (m, 2H), 7.97 (s, 1H), 8.42 (s, 1H), 8.88 (s, 1H), 11.20 (s, 1H); LCMS (ESI) m/z 392.1 [M+H]⁺.

### Examples 148 and 149

### (R)—N²-(1-(methylsulfonyl)-1H-indazol-4-yl)-N-(tetrahydrofuran-3-yl)-5-(trifluoromethyl)pyrimidine-2,4-diamine (Example 148) and (S)—N²-(1-(Methylsulfonyl)-1H-indazol-4-yl)-N⁴-(tetrahydrofuran-3-yl)-5-(trifluoromethyl)pyrimidine-2,4-diamine (Example 149)

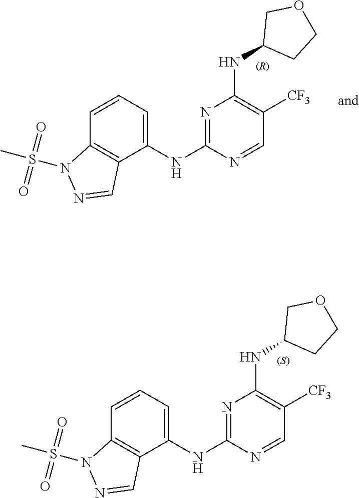

### Step 1: 2-chloro-N-(tetrahydrofuran-3-yl)-5-(trifluoromethyl)pyrimidin-4-amine

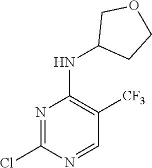 [SMILES: FC(F)(F)c1cnc(Cl)nc1NC1CCOC1]

To a solution of 2,4-dichloro-5-(trifluoromethyl)pyrimidine (500 mg, 2.30 mmol, 1 eq) in MeCN (5 mL) was added K₂CO₃ (636 mg, 4.61 mmol, 2 eq) and tetrahydrofuran-3-amine (284 mg, 2.30 mmol, 1 eq, HCl). The mixture was degassed and purged with N₂ for three times, then was stirred at 0° C. for 3 hours under N₂ atmosphere. The reaction mixture was concentrated under reduced pressure to give a residue. The residue was diluted with H₂O (10 mL) and extracted with EtOAc (15 mL×3). The combined organic layers were washed with brine (10 mL), dried over Na₂SO₄, filtered and concentrated under reduced pressure to give a residue. The residue was purified by flash chromatography (ISCO®; 12 g SepaFlash® Silica Flash Column, petroleum ether/EtOAc with EtOAc from 0-15%, 40 mL/min, 254 mn/I₂/KMnO₄/PMA) to provide 2-chloro-N-tetrahydrofuran-3-yl-5-(trifluoromethyl)pyrimidin-4-amine (Intermediate 148-a) as a white solid (240 mg, 18.4%). LCMS (ESI) m/z 267.9 [M+H]⁺.

### Step 2: N²-(1-(methylsulfonyl)-1H-indazol-4-yl)-N⁴-(tetrahydrofuran-3-yl)-5-(trifluoromethyl)pyrimidine-2,4-diamine

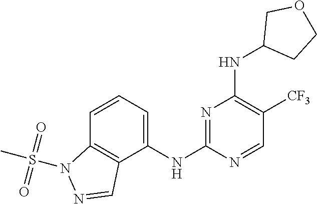 [SMILES: CS(=O)(=O)n1ncc2c(Nc3ncc(C(F)(F)F)c(NC4CCOC4)n3)cccc21]

To a solution of 1-methylsulfonylindazol-4-amine (Intermediate 101-b) (100 mg, 0.473 mmol, 1 eq) in IPA (1 mL) was added 2-chloro-N-tetrahydrofuran-3-yl-5-(trifluoromethyl)pyrimidin-4-amine (Intermediate 148-a) (126 mug, 0.473 mmol, 1 eq) and HC (1 M, 0.710 mL, 1.5 eq). The mixture was degassed and purged with N₂ for three times, stirred at 80° C. for 4 hours under N₂ atmosphere. The mixture was basified at 0° C. with NaHCO₃ (aq) to pH=6-7 and extracted with EtOAc (10 mL×3). The combined organic layers were washed with brine (10 mL), dried by Na₂SO₄, filtered and concentrated under reduced pressure to give a residue. The residue was purified by flash chromatography (ISCO®; 4 g SepaFlash® Silica Flash Column, petroleum ether/EtOAc with EtOAc from 0-50%, 30 mL/min, 254 mn/I₂/KMnO₄/PMA) to provide racemic N²-methylsulfonylindazol-4-yl)-N4-tetrahydrofuran-3-yl-5-(trifluoromethyl)pyrimidine-2,4-diamine (Intermediate 148-b) as a white solid (100 mg, 45.3%). LCMS (ESI) m/z 443.0 [M+H]⁺.

### Step 3: (R)—N²-(1-(methylsulfonyl)-1H-indazol-4-yl)-N⁴-(tetrahydrofuran-3-yl)-5-(trifluoromethyl)pyrimidine-2,4-diamine (Example 148) and (S)—N²-(1-(methylsulfonyl)-1H-indazol-4-yl)-N⁴-(tetrahydrofuran-3-yl)-5-(trifluoromethyl)pyrimidine-2,4-diamine (Example 149)

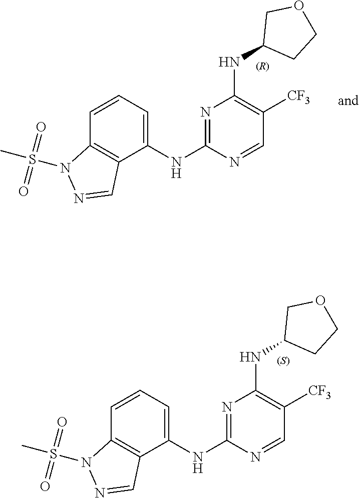

The racemic (Intermediate 148-b) was purified by SFC (i-PrOH (0.1% NH₃H₂O)/75% CO₂ on DAICEL CHIRALPAK IG (250 mm×50 mm×10 μm) at 200 mL/min FR; BRP=100 bar) to provide the two fractions, which after removal of solvents, provided the following two products respectively:

Fraction A (47.04 mg. RT=1.692 min, @ (column: DAICEL CHIRALPAK AD (250 mm×30 mm×10 μm); mobile phase: [0.1% NH₃H₂O EtOH]; B %: 40%-40%, min as additive) was tentatively assigned as compound Example 148: ¹H NMR (400 MHz, CD₃OD) δ ppm 1.94-2.03 (m, 1H), 2.20-2.32 (m, 1H), 3.29 (s, 3H), 3.64 (dd, J=9.03, 4.52 Hz, 1H), 3.78 (td, J=8.28, 6.27 Hz, 1H), 3.85-3.98 (m, 1H), 3.85-3.98 (m, 1H), 4.68 (br d, J=7.28 Hz, 1H), 7.55-7.62 (m, 1H), 7.78 (d, J=8.53 Hz, 1H), 7.82 (d, J=7.78 Hz, 1H), 8.20 (s, 1H), 8.54 (s, 1H). ¹⁹F NMR (377 MHz, CD₃OD) δ ppm −63.26 (br s, 3F). LCMS (ESI) m/z 443.0 [M+H]⁺.

Fraction B (41.15 mg, RT=1.956 min, @ (column: DAICEL CHIRALPAK AD (250 mm×30 mm×10 um); mobile phase: [0.1% NH₃H₂O EtOH]; B %: 40%-40%, min as additive) was tentatively assigned as compound Example 149: H NMR (400 MHz, CD₃OD) δ ppm 1.95-2.02 (m, 1H), 2.21-2.28 (m, 1H), 3.29 (s, 3H), 3.64 (dd, J=9.16, 4.39 Hz, 1H), 3.75-3.81 (m, 1H), 3.84-3.95 (m, 2H), 4.68 (br s, 1H), 7.56-7.61 (n, 1H), 7.78 (d, J=8.53 Hz, 1H), 7.82 (d, J=7.78 Hz, 1H), 8.20 (s, 1H), 8.55 (d, J=0.75 Hz, 1H). ³⁹F NMR (377 MHz, CD₃OD) δ ppm −63.26 (br s, 3F). LCMS (ESI) m/z 443.0 [M+H]⁺.

### Example 150

### N⁴-cyclobutyl-N²-(1-methylsulfonylindazol-4-yl)-5-(trifluoromethyl)pyrimidine-2,4-diamine

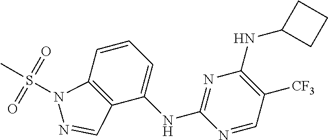 [SMILES: CS(=O)(=O)n1ncc2c(Nc3ncc(C(F)(F)F)c(NC4CCC4)n3)cccc21]

### Step 1: 2-chloro-N-cyclobutyl-5-(trifluoromethyl)pyrimidin-4-amine

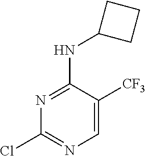 [SMILES: FC(F)(F)c1cnc(Cl)nc1NC1CCC1]

To a solution of 2,4-dichloro-5-(trifluoromethyl)pyrimidine (5 g, 23.04 mmol 1 eq in MeCN (60 mL) was added K₂CO₃ (6.37 g, 46.09 mmol, 2 eq) and cyclobutanamine (1.80 g, 25.35 mmol, 2.17 mL, 1.1 eq). The mixture was stirred at 20° C. for 2 hrs, then concentrated under reduced pressure. The residue was purified by prep-HPLC (column: Welch Xtimate C18 100×40 mm×3 μm; mobile phase: [water(HCl)-ACN]; B %: 56%-86%, 8 min) to give 2-chloro-N-cyclobutyl-5-(trifluoromethyl)pyrimidin-4-amine (Intermediate 150-a) as a white solid (1.08 g, 36.13%). LCMS (ESI) m/z 252.0 [M+H]⁺.

### Step 2: N⁴-cyclobutyl-N²-(1-methylsulfonylindazol-4-yl)-5-(trifluoromethyl)pyrimidine-2,4-diamine

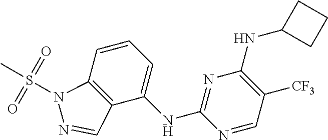 [SMILES: CS(=O)(=O)n1ncc2c(Nc3ncc(C(F)(F)F)c(NC4CCC4)n3)cccc21]

To a solution of 2-chloro-N-cyclobutyl-5-(trifluoromethyl)pyrimidin-4-amine (Intermediate 150-a) (0.3 g, 1.19 mmol, 1 eq) in IPA (6 mL) was added HCl (1 M, 1.79 mL) and 1-methylsulfonyl-1H-indazol-4-amine (Intermediate 101-b) (0.264 g, 1.25 mmol, 1.05 eq). The mixture was stirred at 80° C. for 4 hrs, then quenched by 10% aq. K₂CO₃ (10 mL) and extracted with EtOAc (10 mL×3). The combined organic layers were washed with brine (20 mL), dried with anhydrous Na₂SO₄, filtered and concentrated under reduced pressure to give a residue. The residue was purified by prep-HPLC (column: Welch Xtimate C18 100×40 mm×3 um; mobile phase: [water (HCl)-ACN]; B %: 56%-86%, 8 min) to provide the title compound as a white solid (0.362 g, 70.52%). ¹H NMR (400 MHz, DMSO-d₆) δ ppm 1.55-1.77 (m, 2H), 2.11-2.28 (m, 4H), 3.41-3.52 (m, 3H), 4.54-4.70 (m, 1H), 7.05-7.20 (m, 1H), 7.48-7.69 (m, 2H), 7.95-8.08 (m, 1H), 8.28 (s, 1H), 8.89 (s, 1H), 10.04 (s, 1H); ¹⁹F NMR (377 MHz, DMSO-d₆) δ ppm −60.10 (br s, 1F): LCMS (ESI) m/z 427.1 [M+H]⁺.

### Example 151

### 5-chloro-N⁴-ethyl-N²-(1-methylsulfonylindazol-4-yl)-7H-pyrrolo[2,3-d]pyrimidine-2,4-diamine

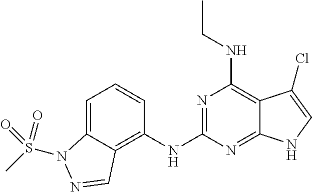 [SMILES: CCNc1nc(Nc2cccc3c2cnn3S(C)(=O)=O)nc2[nH]cc(Cl)c12]

A mixture of 1-methylsulfonyl-1H-indazol-4-amine (Intermediate 101-b) (365 mg, 1.73 mmol), 2,5-dichloro-N-ethyl-7H-pyrrolo[2,3-d]pyrimidin-4-amine (Intermediate 147-b) (200 mg, 865 μmol) and TFA (492 mg, 4.32 mmol L) in TFE (10 mL) was stirred at 100° C. for 16 hrs. TFE was evaporated. The residue was purified by prep-HPLC (column: Boston Green ODS 150×30 mm×5 μm; mobile phase: [water (HCl)-ACN]; B %: 30%-70%, 9 min) to provide the title compound as a white solid (28.8 mg, 8.20% yield). ¹H NMR (400 MHz, DMSO-d₆) δ ppm 1.25 (t, J=7.09 Hz, 3H), 3.47 (s, 3H), 3.68 (br s, 2H), 7.06 (s, 1H), 7.54-7.70 (m, 2H), 7.94-8.39 (m, 1H), 9.09 (s, 1H), 10.73 (br s, 1H), 12.16 (br s, 1H); LCM S (ESI) m/z 406.0 [M+H]⁺.

### Example 152

### 5-chloro-N4-ethyl-N²-(1-(methylsulfonyl)-1H-indazol-4-yl)pyrimidine-2,4-diamine

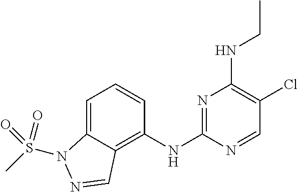 [SMILES: CCNc1nc(Nc2cccc3c2cnn3S(C)(=O)=O)ncc1Cl]

A mixture of 1-methylsulfonyl-1H-indazol-4-amine (Intermediate 101-b) (60 mg, 0.284 mmol, 1 eq), 2,5-dichloro-N-ethyl-pyrimidin-4-amine (Intermediate 70-a) (54.55 mg, 0.284 mmol, 1 eq) and HCl (1 M, 0.426 mL, 1.5 eq) in IPA (0.5 mL) was degassed and purged with N₂ for 3 times, and then stirred at 80° C. for 4 hr under N₂ atmosphere. The reaction mixture was quenched by aq. NaHCO₃ (10 mL) and extracted with EtOAc (10 mL×3). The combined organic layers were dried with anhydrous Na₂SO₄, filtered and concentrated under reduced pressure to give a residue, which was purified by flash silica gel chromatography (iSCO®; 12 g SepaFlash® Silica Flash Column, Eluent of 0-30% Ethyl acetate/Petroleum ethergradient @ 30 mL/min) to provide the title compound as a yellow solid (54.77 mg, 45% yield). ¹H NMR (400 MHz, DMSO-d₆) δ ppm 1.18 (t, 3H), 3.33 (s, 3H), 3.43-3.48 (m, 2H), 7.26-7.39 (m, 1H), 7.52 (d, J=4.77 Hz, 2H), 7.99 (s, 1H), 8.06-8.16 (m, 1H), 8.90 (s, 1H), 9.64 (s, 1H); LCMS (ESI) m/z 367.0 [M+H]⁺.

### Example 153

### N⁴-cyclopropyl-N²-(1-(cyclopropylsulfonyl)-7-fluoro-1H-indazol-4-yl)-5-(trifluoromethyl) pyrimidine-2,4-diamine

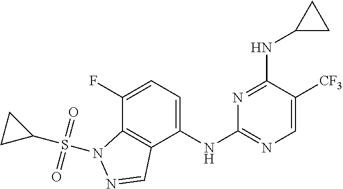 [SMILES: O=S(=O)(C1CC1)n1ncc2c(Nc3ncc(C(F)(F)F)c(NC4CC4)n3)ccc(F)c21]

### Step 1: 4-bromo-1-(cyclopropylsulfonyl)-7-fluoro-1H-indazole

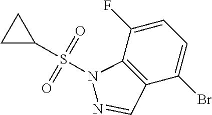 [SMILES: O=S(=O)(C1CC1)n1ncc2c(Br)ccc(F)c21]

To a solution of 4-bromo-7-fluoro-1H-indazole (1 g, 4.65 mmol, 1 eq) in TH-F (10 mL) was added t-BuOK (521.8 mg, 4.65 mmol, 1 eq) and the mixture was stirred at 25° C. for 0.5 h. Then cyclopropanesulfonyl chloride (653.8 mg, 4.65 mmol, 1 eq) was added to the above mixture and the resulting reaction mixture was stirred at 25° C. for 0.5 hr. The residue was diluted with H₂O (20 mL) and extracted with EtOAc (40 mL×3). The combined organic layers were washed with brine (30 mL), dried over Na₂SO₄, filtered and concentrated under reduced pressure to give a residue. The residue was purified by prep-HPLC (column: Welch Xtimate C18 100×40 mm×3 mm; mobile phase: [water (HCl)-ACN]; B %: 45%-75%, 8 min) to give compound 4-bromo-1-cyclopropylsulfonyl-7-fluoro-indazole (Intermediate 153-a) as a white solid (90 mg, 6.06%). ¹H NMR (400 MHz, CDCl₃) ppm 1.07-1.24 (m, 2H), 1.48-1.60 (m, 2H), 2.84-3.03 (m, 1H), 7.19 (dd, J=11.04, 8.28 Hz, 1H), 7.44 (dd, J=8.28, 3.01 Hz, 1H), 8.27 (s, 1H).

### Step 2: 1-(cyclopropylsulfonyl)-N-(diphenylmethylene)-7-fluoro-1H-indazol-4-amine

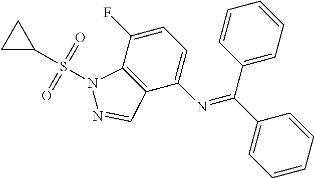 [SMILES: O=S(=O)(C1CC1)n1ncc2c(N=C(c3ccccc3)c3ccccc3)ccc(F)c21]

To a solution of 4-bromo-1-cyclopropylsulfonyl-7-fluoro-indazole (Intermediate 153-a) (70 mg, 0.22 mmol, 1 eq) in dioxane (1 mL) was added Cs₂CO₃ (171.5 mg, 0.53 mmol, 2.4 eq), diphenylmethanimine (51.7 mg, 0.26 mmol, 1.3 eq) and Xantphos Pd G4 (21.1 mg, 0.02 mmol, 0.1 eq). The mixture was degassed and purged with N₂ for three times, then stirred at 90° C. for 2 hrs under N₂ atmosphere. The reaction mixture was concentrated under reduced pressure to give a residue. The residue was purified by flash chromatography (ISCO®; 12 g SepaFlash® Silica Flash Column, petroleum ether/EtOAc with EtOAc from 0-30%, 30 mL/min, 254 mn) to give N-(1-cyclopropylsulfonyl-7-fluoro-indazol-4-yl)-1,1-diphenyl-methanimine (Intermediate 153-b) as a yellow solid (90 mg, 90%). LCMS (ESI) m/z 420.0 [M+H]⁺.

### Step 3: 1-(cyclopropylsulfonyl)-7-fluoro-1H-indazol-4-amine

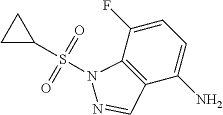 [SMILES: Nc1ccc(F)c2c1cnn2S(=O)(=O)C1CC1]

A solution of N-(1-cyclopropylsulfonyl-7-fluoro-indazol-4-yl)-1,1-diphenyl-methanimine is (Intermediate 153-b) (90 mg, 0.21 mmol, 1 eq) in HCl/dioxane (2 ml) was stirred at 25° C. for 0.5 h. The reaction mixture was concentrated under reduced pressure to give compound 1-cyclopropylsulfonyl-7-fluoro-indazol-4-amine (Intermediate 153-c) as a white solid (80 mg, 89.1%).

### Step 4: N⁴-cyclopropyl-N²-(1-(cyclopropylsulfonyl)-7-fluoro-1H-indazol-4-yl)-5-(trifluoromethyl) pyrimidine-2,4-diamine

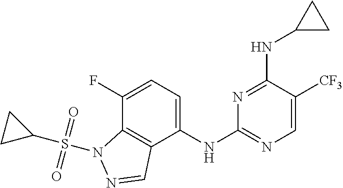 [SMILES: O=S(=O)(C1CC1)n1ncc2c(Nc3ncc(C(F)(F)F)c(NC4CC4)n3)ccc(F)c21]

To a solution of 1-cyclopropylsulfonyl-7-fluoro-indazol-4-amine (Intermediate 153-c) (60 mg, 0.24 mmol, 1 eq) in i-PrOH (2 mL) was added HCl (1 M, 0.3 mL, 1.5 eq) and 2-chloro-N-cyclopropyl-5-(trifluoromethyl)pyrimidin-4-amine (Intermediate 68-a) (61.4 mg, 0.26 mmol, 1.1 eq). The mixture was stirred at 80° C. for 2 hrs and concentrated under reduced pressure to give a residue. The residue was purified by prep-HPLC (column: Phenomenex Synergi C18 100×30 mm×4 um; mobile phase: [water (HCl)-ACN]; B %: 40%-70%, 7.8 min) to give the title compound as a white solid (45.18 mg, 42.1%). ¹H NMR (400 MHz, DMSO-d₆) δ 0.56-0.69 (m, 2H), 0.70-0.79 (m, 2H), 1.11-1.20 (m, 2H), 1.22-1.32 (m, 2H), 2.74-2.86 (m, 1H), 3.10-3.25 (m, 1H), 7.53 (dd, J=11.54, 8.78 Hz, 1H), 7.59-7.72 (m, 1H), 8.13-8.26 (m, 1H), 8.31 (s, 1H), 8.95 (s, 1H), 10.45 (s, 1H). ¹⁹F NMR (377 MHz, DMSO-d₆) δ ppm −60.49 (s, 3F), −127.10 (s, 1F). LCMS (ESI) m/z 457.0 [M+H]⁺.

### Example 154

### 2-(4-((4-(ethylamino)-5-(trifluoromethyl)pyrimidin-2-yl)amino)-1H-indazol-1-yl)-2-methylpropanenitrile

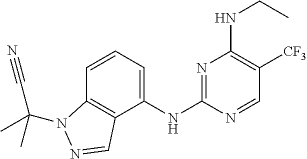 [SMILES: CCNc1nc(Nc2cccc3c2cnn3C(C)(C)C#N)ncc1C(F)(F)F]

A mixture of 2-(4-aminoindazol-1-yl)-2-methyl-propanenitrile (Intermediate 31-d) (150 mg, 749 μmol), 2-chloro-N-ethyl-5-(trifluoromethyl)pyrimidin-4-amine (Intermediate 69-a) (120 mg, 532 μmol) and HCl (1.04 mL, 292 μmol, 1M) in i-PrOH (1 mL) was stirred at 80° C. for 1 hr. The reaction was purified by prep-HPLC (column: C18 (250×50 mm×10 μm); mobile phase: [water (NH₃H₂O+NH₄HCO₃)-ACN]; B %: 40%-70%, 9 min) to provide the title compound as a white solid (30 mg, 14.5% yield). ¹H NMR (400 MHz, DMSO-d₆) δ ppm 1.11-1.20 (m, 3H), 2.05-2.13 (m, 6H), 3.47-3.52 (m, 2H), 7.18-7.28 (m, 1H), 7.40-7.52 (m, 2H), 7.98 (s, 1H), 8.24 (s, 1H), 8.55 (s, 1H), 9.82-9.92 (m, 1H); LCMS (ESI) m/z 390.1 [M+H]⁺.

### Example 155

### 2-[4-[[4-(2-methoxyethylamino)-5-(trifluoromethyl)pyrimidin-2-yl]amino]indazol-1-yl]-2-methyl-propanenitrile

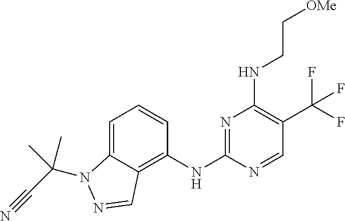 [SMILES: COCCNc1nc(Nc2cccc3c2cnn3C(C)(C)C#N)ncc1C(F)(F)F]

### Step 1: 2-chloro-N-(2-methoxyethyl)-5-(trifluoromethyl)pyrimidin-4-amine

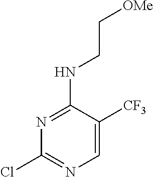 [SMILES: COCCNc1nc(Cl)ncc1C(F)(F)F]

To a solution of 2,4-dichloro-5-(trifluoromethyl)pyrimidine (1 g, 4.61 mmol, 1 eq) in MeCN (12 mL) was added K₂CO₃ (0.955 g, 6.91 mmol, 1.5 eq) and 2-methoxyethanamine (0.38 g, 5.07 mmol, 0.44 mL, 1.1 eq). The mixture was stirred at 20° C. for 2 hrs and concentrated under reduced pressure. The residue was purified by prep-HPLC (column: Welch Xtimate C18 100×40 mm×3 μm; mobile phase: [water (HCl)-ACN]; 13%: 56%-86%, 8 min) to give 2-chloro-N-(2-methoxyethyl)-5-(trifluoromethyl)pyrimidin-4-amine (Intermediate 155-a) as a white solid (0.423 g, 32.31%). ¹H NMR (400 MHz, DMSO-d₆) δ (ppm 3.26 (s, 3H), 3.46-3.53 (m, 2H), 3.57-3.64 (m, 2H), 7.89 (br s, 1H), 8.39 (s, 1H); LCMS (ESI) m/z 256.0 [M+H]⁺.

### Step 2: 2-[4-[[4-(2-methoxyethylamino)-5-(trifluoromethyl) pyrimidin-2-yl]amino]indazol-1-yl]-2-methyl-propanenitrile

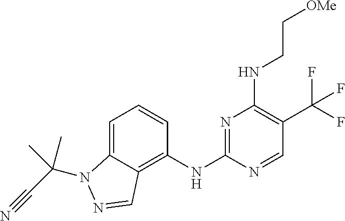 [SMILES: COCCNc1nc(Nc2cccc3c2cnn3C(C)(C)C#N)ncc1C(F)(F)F]

A mixture of 2-(4-aminoindazol-1-yl)-2-methyl-propanenitrile (Intermediate 31-d) (50 mg, 0.25 mmol, 1 eq), 2-chloro-N-(2-methoxyethyl)-5-(trifluoromethyl)pyrimidin-4-amine (Intermediate 155-a) (63.83 mg, 0.25 mmol, 1 eq) and HCl (1 M, 0.37 mL, 1.5 eq) in IPA (2 mL) was degassed and purged with N₂ for 3 times, and then stirred at 80° C. for 4 hrs under N₂ atmosphere. The reaction mixture was concentrated under reduced pressure. The residue was diluted with H₂O (5 mL), neutralized to pH=7˜8 by 1M aq. NaHCO₃ and extracted with EtOAc (10 mL×3), The combined organic layers were washed with brine (10 mL), dried with anhydrous Na₂SO₄, filtered and concentrated under reduced pressure to give a residue. The residue was purified by prep-HPLC (column: 2_Phenomenex Gemini C18 75×40 mm×3 Lim; mobile phase: [water (NH₃H₂O)-ACN]; B %: 50%-80%, 7 min) to provide the title compound as a white solid (11.25 mg, 10.73%). ¹H NMR (400 M-z, CD₃OD) δ ppm 2.14 (s, 6H), 334-336 (m, 3H) 3.53-3.59 (m, 2H), 369 (s, 2H), 7.45-7.51 (m, 1H), 7.57-7.63 (m, 1H), 7.80-7.85 (m, 1H), 8.18 (s, 1H), 8.24 (s, 1H), ¹⁹F NMR (377 MHz, CD₃OD) δ ppm −63.51 (br d, J=20 Hz, 3F) LCMS (ESI) m/z 420.1 [M+H]⁺.

### Example 156

### 2-(4-((5-chloro-4-(ethylamino)-7H-pyrrolo[2,3-d]pyrimidin-2-yl)amino)-1H-indazol-1-yl)-2-methylpropanenitrile

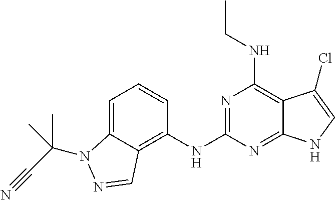 [SMILES: CCNc1nc(Nc2cccc3c2cnn3C(C)(C)C#N)nc2[nH]cc(Cl)c12]

A mixture of 2-(4-aminoindazol-1-yl)-2-methyl-propanenitrile (Intermediate 31-d) (100 mg, 499 μmol), 2,5-dichloro-N-ethyl-7H-pyrrolo[2,3-d]pyrimidin-4-amine (Intermediate 147-b) (115 mg, 499 μmol) and TFA (40 μL, 540 μmol) in TFE (3 mL) was stirred at 90° C. for 16 hrs under N₂ atmosphere. The reaction mixture was purified by prep-HPLC (column: C18 (250×50 mm×10 μm); mobile phase: [water (NH₃H₂O+NH₄HCO₃)-ACN]; B %: 38%-78%, 9 min) to provide the title compound as a yellow solid (10 mg, 5.1% yield). ¹H NMR (400 MHz, CD₃OD) δ (ppm 1.33 (t, 3H), 2.12-2.15 (m, 6H), 3.62-3.69 (m, 2H), 6.83 (s, 1H), 7.38-7.52 (m, 2H), 8.09-8.16 (m, 1H), 8.30 (s, 1H); LCMS (ESI) m/z 395.1 [M+H]⁺.

### Example 157

### 2-[4-[[4-[(3-hydroxycyclobutyl)amino]-5-(trifluoromethyl)pyrimidin-2-yl]amino]indazol-1-yl]-2-methyl-propanenitrile

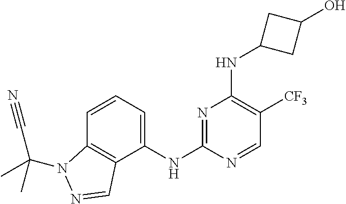 [SMILES: CC(C)(C#N)n1ncc2c(Nc3ncc(C(F)(F)F)c(NC4CC(O)C4)n3)cccc21]

### Step 1: 3-[[2-chloro-5-(trifluoromethyl)pyrimidin-4-yl]amino]cyclobutanol

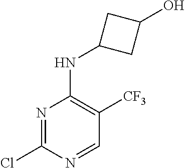 [SMILES: OC1CC(Nc2nc(Cl)ncc2C(F)(F)F)C1]

A mixture of 2,4-dichloro-5-(trifluoromethyl)pyrimidine (500 mg, 2.30 mmol, 1 eq), 3-aminocyclobutanol (200.76 mg, 2.30 mmol, 1 eq), K₂CO₃ (636.96 mg, 4.61 mmol, 2 eq) in MeCN (5 mL) was degassed and purged with N₂ for 3 times, and then stirred at 0° C. for 2 hrs under N₂ atmosphere. The residue was diluted with H₂O (10 ml) and extracted with EtOAc (15 mL×3). The combined organic layers were washed with brine (10 mL), dried over Na₂SO₄, filtered and concentrated under reduced pressure to give a residue. The residue was purified by flash silica gel chromatography (ISCO®; 20 g SepaFlash® Silica Flash Column, Eluent of 0˜30% petroleum ether/EtOAc @ 40 mL/min) to give 3-[[2-chloro-5-(trifluoromethyl)pyrimidin-4-yl]amino]cyclobutanol (Intermediate 157-a) as a white solid (90 mg, 14.2%).

### Step 2: 2-[4-[[4-[(3-hydroxycyclobutyl)amino]-5-(trifluoromethyl)pyrimidin-2-yl]amino]indazol-1-yl]-2-methyl-propanenitrile

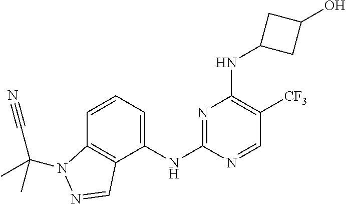 [SMILES: CC(C)(C#N)n1ncc2c(Nc3ncc(C(F)(F)F)c(NC4CC(O)C4)n3)cccc21]

A mixture of 2-(4-aminoindazol-1-yl)-2-methyl-propanenitrile (Intermediate 31-d) (50 mg, 0.25 mmol, 1 eq), 3-[[2-chloro-5-(trifluoromethyl)pyrimidin-4-yl]amino]cyclobutanol (Intermediate 157-a) (66.83 mg, 0.25 mmol, 1 eq) and HCl (1 M, 0.374 mL, 1.5 eq) in IPA (1 mL) was degassed and purged with N₂ for 3 times, and then stirred at 80° C. for 2 hrs under N₂ atmosphere. The residue was quenched by aq. NaHCO₃ (10 mL) and extracted with EtOAc (15 mL×3). The combined organic layers were washed with brine (10 mL), dried over Na₂SO₄, filtered and concentrated under reduced pressure to give a residue, which was purified by prep-HPLC (column: Phenomenex Synergi C18 100×30 mm×4 Lim; mobile phase: [water(HCl)-ACN]; B %: 23%-53%, 9.5 min) to provide the title compound as a white solid (38 mg, 31.88%). ¹H NMR (400 MHz, DMSO-d₆) δ ppm 1.97-2.05 (m, 2H), 2.10 (s, 6H), 2.52 (br d, J=2.0 Hz, 2H), 3.76-3.84 (m, 1H), 4.00 (br d, J=6.4 Hz, 1H), 7.47-7.54 (m, 1H), 7.60 (br d, J=8.4 Hz, 1H), 781 (d, J=7.6 Hz, 1H), 8.33 (s, 1H), 8.48 (s, 1H), 10.32 (br s, 1H); ¹⁹F NMR (376 MHz, DMSO-d₆) δ ppm −60.44; LCMS (ESI) m/z 432.1 [M+H]⁺.

### Example 158

### 5-chloro-4-ethoxy-N-(1-methylsulfonylindazol-4-yl)-7H-pyrrolo [2,3-d]pyrimidin-2-amine

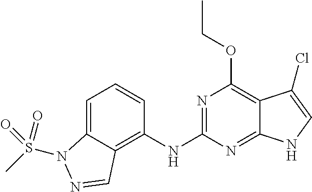 [SMILES: CCOc1nc(Nc2cccc3c2cnn3S(C)(=O)=O)nc2[nH]cc(Cl)c12]

### Step 1: 2,4,5-trichloro-71H-pyrrolo[2,3-d]pyrimidine

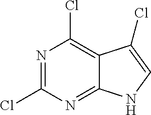 [SMILES: Clc1nc(Cl)c2c(Cl)c[nH]c2n1]

To a solution of 2,4-dichloro-7H-pyrrolo[2,3-d]pyrimidine (10 g, 53.1 mmol) in THF (100 mL) was added NCS (8.5 g, 63.6 mmol) and the reaction mixture was stirred at 90° C. for 4 hours. The reaction mixture was concentrated under reduced pressure to remove solvent. The residue was diluted with aq. NaHCO₃ (100 mL) and extracted with EtOAc (100 mL×2). The combined organic layers were washed with brine (100 mL×2), dried over anhydrous Na₂SO₄, filtered and concentrated under reduced pressure to give 2,4,5-trichloro-7H-pyrrolo[2,3-d]pyrimidine (intermediate 158-a) as a purple solid (12 g, crude). ¹H NMR (400 MHz, DMSO-d₆) δ ppm 7.93 (s, 1H), 13.07 (br s, 1H); LCMS (ESI) m/z 221.9, 223.9 [M+H]⁺.

### Step 2: 2,5-dichloro-4-ethoxy-711-pyrrolo[2,3-d]pyrimidine

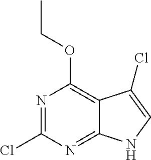

To a solution of 2,4,5-trichloro-7H-pyrrolo[2,3-d]pyrimidine (Intermediate 158-a) (11 g, 49.4 mmol) in EtOH (150 mL) was added NaOEt (7.5 g, 110 mmol) and the reaction mixture was stirred at 90° C. for 12 hours. The reaction mixture was concentrated under reduced pressure, diluted with 50 mL water and extracted with EtOAc (50 mL×2). The combined organic layers were washed with brine (50 mL×2), dried over anhydrous Na₂SO₄, filtered and concentrated under reduced pressure to give a residue. The residue was purified by flash silica gel chromatography (ISCO®; 20 g SepaFlash® Silica Flash Column, petroleum ether/EtOAc with EtOAc from 0˜40%, flow rate: 40 mL/min, 254 nm) to furnish 2,5-dichloro-4-ethoxy-7H-pyrrolo[2,3-d]pyrimidine (Intermediate 158-b) as a white solid (11 g, 86.2% yield). ¹H NMR (400 MHz, DMSO-d₆) δ (ppm 138 (t, J=7.03 Hz, 3H), 4.51 (q, J=7.11 Hz, 2H), 7.54 (s, 1H).

### Step 3: 2-[(2,5-dichloro-4-ethoxy-pyrrolo[2,3-d]pyrimidin-7-yl)methoxy]ethyl-trimethyl-silane

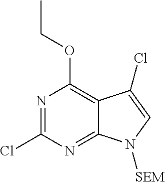 [SMILES: CCOc1nc(Cl)nc2c1c(Cl)cn2COCC[Si](C)(C)C]

A mixture of 2,5-dichloro-4-ethoxy-7H-pyrrolo[2,3-d]pyrimidine (Intermediate 158-b) (1 g, 4.31 mmol), SEM-Cl (870 mg, 5.22 mmol), NaH (210 mg, 5.25 mmol, 60% purity) in THF (20 mL) was degassed and purged with N₂ for 3 times, then stirred at 0° C. for 1 hr under N₂ atmosphere. The reaction mixture was quenched by H₂O (50 mL) at 25° C., and then extracted with EtOAc (50 mL×3). The combined organic layers were washed with brine (50 mL×3), dried over anhydrous Na₂SO₄, filtered and concentrated under reduced pressure. The residue was purified by flash chromatography (ISCO®; 20 g SepaFlash® Silica Flash Column, petroleum ether/EtOAc with EtOAc from 0˜20%, Flow Rate: 85 mL/min, 254 nm) to provide 2-[(2,5-dichloro-4-ethoxy-pyrrolo[2,3-d]pyrimidin-7-yl)methoxy]ethyl-trimethyl-silane as a white solid (Intermediate 158-c) (1 g, 64.1% yield). 1H NMR (400 MHz, CDCl₃) δ ppm −0.04-0.01 (m, 9H), 0.90-1.02 (m, 2H), 1.51 (t, J=7.15 Hz, 3H), 3.37-3.75 (m, 2H), 4.48-4.83 (m, 2H), 5.32-5.74 (m, 2H), 7.54 (s, 1H); LCMS (ESI) m/z 362.0 [M+H]⁺.

### Step 4: 5-chloro-4-ethoxy-N-(1-methylsulfonylindazol-4-yl)-7-(2-trimethylsilylethoxymethyl)pyrrolo[2,3-d]pyrimidin-2-amine

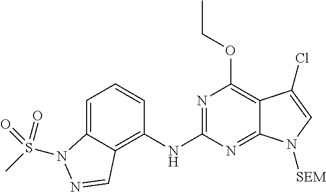 [SMILES: CCOc1nc(Nc2cccc3c2cnn3S(C)(=O)=O)nc2c1c(Cl)cn2COCC[Si](C)(C)C]

To a solution of 1-methylsulfonylindazol-4-amine (Intermediate 101-b) (95 mg, 449 μmol) and 2-[(2,5-dichloro-4-ethoxy-pyrrolo[2,3-d]pyrimidin-7-yl)methoxy]ethyl-trimethyl-silane (Intermediate 158-c) (162 mg, 449 μmol) in toluene (2 mL) was added Pd₂(dba)₃ (41.1 mg, 44.9 μmol), Xantphos (52.0 mg, 89.9 μmol) and Cs₂CO₃ (366 mg, 1.12 mmol). The mixture was stirred at 100° C. for 16 hrs under N₂ atmosphere, then concentrated and purified by flash chromatography (ISCO®; 4 g SepaFlash® Silica Flash Column, petroleum ether/EtOAc with EtOAc from 0-50%, 12 mL/min, 254 nm) to give 5-chloro-4-ethoxy-N-(1-methylsulfonylindazol-4-yl)-7-(2-trimethylsilylethoxymethyl)pyrrolo[2,3-d]pyrimidin-2-amine (Intermediate 158-d) as a brown solid (70 mg, 29.0% yield). ¹H NMR (400 MHz, DMSO-d₆) δ ppm −0.14-0.07 (m, 9H), 0.76-0.82 (m, 2H), 1.32-1.37 (m, 3H), 3.09 (s, 3H), 3.43-3.54 (m, 2H), 4.43-4.51 (m, 2H), 5.32-5.46 (m, 2H), 7.15-7.29 (m, 2H), 7.35 (s, 1H), 7.57-767 (m, 2H), 936 (br s, 1H).

### Step 5: 5-chloro-4-ethoxy-N-(1-methylsulfonylindazol-4-yl)-7H-pyrrolo [2,3-d]pyrimidin-2-amine

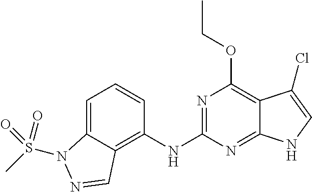 [SMILES: CCOc1nc(Nc2cccc3c2cnn3S(C)(=O)=O)nc2[nH]cc(Cl)c12]

To a solution of 5-chloro-4-ethoxy-N-(1-methylsulfonylindazol-4-yl)-7-(2-trimethylsilylethoxy methyl)pyrrolo[2,3-d]pyrimidin-2-amine (Intermediate 158-d) (60 ng, 111 μmol) in 4M HCl/dioxane (2 mL) was added Et₃SiH (38.9 mg, 335 μmol). The mixture was stirred at 25° C. for 16 hrs. The residue was purified by Pre-HPLC (column: Phenomenex C18 75×30 mm×3 μm; mobile phase: [water (ammonia hydroxide v/v)-ACN]; B %: 13%-53%, 9 min) to provide the title compound as a white solid (1.1 mg, 2.42% yield). ¹H NMR (400 MHz, CD₃OD) δ ppm 1.48 (t, J=7.0 Hz, 3H), 3.15 (s, 3H), 4.57 (q, J=7.0 Hz, 2H), 6.96 (s, 1H), 7.19-7.21 (m, 1H), 7.60 (t, J=8.4 Hz, 1H), 8.25 (d, J=8.4 Hz, 1H), 8.31 (s, 1H); LCMS (ESI) m/z 407.1 [M+H]⁺.

### Example 159

### N⁴-cyclopropyl-N²-(1-(cyclopropylsulfonyl)-1H-pyrazolo[3,4-b]pyridin-4-yl)-5-(trifluoromethyl)pyrimidine-2,4-diamine

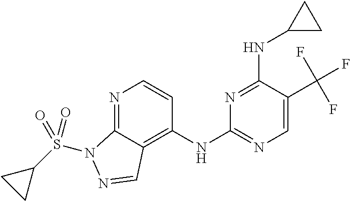 [SMILES: O=S(=O)(C1CC1)n1ncc2c(Nc3ncc(C(F)(F)F)c(NC4CC4)n3)ccnc21]

### Step 1: 4-bromo-1-(cyclopropylsulfonyl)-1H-pyrazolo[3,4-b]pyridine

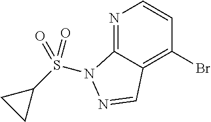 [SMILES: O=S(=O)(C1CC1)n1ncc2c(Br)ccnc21]

To a solution of 4-bromo-1H-pyrazolo[3,4-b]pyridine (200 mg, 1.01 mmol, 1 eq) in THF (3 mL) was added NaH (80 mg, 2.02 mmol, 60% purity, 2 eq) and cyclopropanesulfonyl chloride (283 mg, 2.02 mmol, 2 eq) at 0° C. The mixture was stirred at 0° C. for 2 hours, then quenched by H₂O (20 mL) at 0° C. and extracted with EtOAc (10 mL×3). The combined organic layers were washed with brine (10 ml), dried by Nₐ2SO₄, filtered and concentrated under reduced pressure to give a residue. The residue was purified by flash chromatography (ISCO®; 12 g SepaFlash® Silica Flash Column, petroleum ether/EtOAc with EtOAc from 0˜40%, 40 ml/min, 254 mn/I₂/KMnO₄/PMA) to provided compound 4-bromo-1-cyclopropylsulfonyl-pyrazolo[3,4-b]pyridine (Intermediate 159-a) as a white solid (270 mg, 87.5%). LCMS (ESI) m/z 301.7, 303.7 [M+H]⁺.

### Step 2: 1-(cyclopropylsulfonyl)-N-(diphenylmethylene)-1H-pyrazolo[3,4-b]pyridin-4-amine

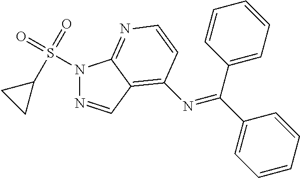 [SMILES: O=S(=O)(C1CC1)n1ncc2c(N=C(c3ccccc3)c3ccccc3)ccnc21]

To a solution of 4-bromo-1-cyclopropylsulfonyl-pyrazolo[3,4-b]pyridine (Intermediate 159-a) (270 mg, 0.893 mmol, 1 eq) in dioxane (3 mL) was added Cs₂CO₃ (698 mg, 2.14 mmol, 2.4 eq), Xantphos Pd G4 (86 mg, 0.089 μmol, 0.1 eq) and diphenylmethanimine (210 mg, 1.16 mmol, 1.3 eq). The mixture was degassed and purged with N₂ for three times, then stirred at 90° C. for 2 hours under N₂ atmosphere. The reaction mixture was concentrated under reduced pressure to give a residue. The residue was diluted with H₂O (10 mL) and extracted with EtOAc (15 mL×3). The combined organic layers were washed with brine (10 mL), dried over Na₂SO₄, filtered and concentrated under reduced pressure to give a residue. The residue was purified by flash chromatography (ISCO®; 4 g SepaFlash® Silica Flash Column, petroleum ether/EtOAc with EtOAc from 0˜40%, 40 mL/min, 254 mn/12/KMnO₄/PMA) to provide N-(1-cyclopropylsulfonylpyrazolo[3,4-b]pyridin-4-yl)-1,1-diphenyl-methanimine (Intermediate 159-b) as a colorless oil (300 mg, 77.5%). LCMS (ESI) m/z 403.0 [M+H]⁺.

### Step 3: 1-(cyclopropylsulfonyl)-1H-pyrazolo[3,4-b]pyridin-4-amine

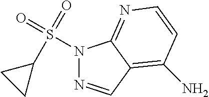 [SMILES: Nc1ccnc2c1cnn2S(=O)(=O)C1CC1]

A solution of N-(1-cyclopropylsulfonylpyrazolo[3,4-b]pyridin-4-yl)-1,1-diphenyl-methanimine (Intermediate 159-b) (300 mg, 0.745 mmol, 1 eq) in HCl/dioxane (3 mL) was stirred at 20° C. for 2 hours. The resulting mixture was basified at 0° C. by aq. NaHCO₃ to pH=7-8 and extracted with EtOAc (10 mL×3). Then the combined organic layers were washed with brine (10 mL), dried by Na₂SO₄, filtered and concentrated under reduced pressure to give a residue. The residue was purified by flash chromatography (ISCO®, 12 g SepaFlash® Silica Flash Column, petroleum ether/EtOAc with EtOAc from 0-100%, 100 mL/min, 254 mn/I₂/KMnO₄/PMA) to provide 1-cyclopropylsulfonylpyrazolo[3,4-b]pyridin-4-amine (Intermediate 159-c) as a white solid (170 mg, 93.8%). LCMS (ESI) m/z 239.0 [M+H]⁺.

### Step 4: N⁴-cyclopropyl-N²-(1-(cyclopropylsulfonyl)-1H-pyrazolo[3,4-b]pyridin-4-yl)-5-(trifluoromethyl)pyrimidine-2,4-diamine

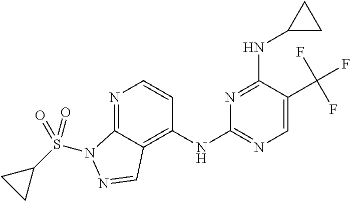 [SMILES: O=S(=O)(C1CC1)n1ncc2c(Nc3ncc(C(F)(F)F)c(NC4CC4)n3)ccnc21]

To a solution of 1-cyclopropylsulfonylpyrazolo[3,4-b]pyridin-4-amine (Intermediate 159-c) (60.17 mg, 0.25 mmol, 1.2 eq) in DMF (1 ml) was added palladium; tritert-butylphosphane (10.75 mg, 21.04 μmol, 0.1 eq), Cs₂CO₃ (205.69 mg, 0.63 mmol, 3 eq) and 2-chloro-N-cyclopropyl-5-(trifluoromethyl)pyrimidin-4-amine (Intermediate 68-a) (50 mg, 0.21 mmol, 1 eq). The mixture was degassed and purged with N₂ for three times, then stirred at 120° C. for 1 hour under N₂ atmosphere. The resulting mixture was diluted with H₂O (10 mL) and extracted with EtOAc (15 mL×3). The combined organic layers were washed with brine (10 mL), dried over Na₂SO₄, filtered and concentrated under reduced pressure to give a residue, which was purified by prep-HPLC (column: Phenomenex Synergi C18 100×30 mm×4 μm; mobile phase: [water (HCl)-ACN]; B %: 40%-70%, 8 min as additive) to provide the title compound as a white solid (15.06 mg, 7.52%). ¹H NMR (400 MHz, CD₃OD) δ ppm 0.74-0.77 (m, 2H), 0.96 (dd, J=7.2, 1.6 Hz, 2H), 1.25 (dd, J=7.6, 1.6 Hz, 2H), 1.51-1.54 (m, 2H), 2.94-2.99 (m, 1H), 3.14-3.20 (m, 1H), 8.41 (s, 1H), 8.54 (d, J=6.4 Hz, 1H), 8.83 (d, J=6.4 Hz, 1H), 8.87 (s, 1H); ¹⁹F NMR (377 MHz, CD₃OD) δ ppm −64.67 (s, 3F); LCMS (ESI) m/z 440.0 [M+H]⁺.

### Example 160

### N⁴-cyclopropyl-N²-(9-oxa-1,2-diazatricyclo[6.4.1.04,13]trideca-2,4,6,8(13)-tetraen-5-yl)-5-(trifluoromethyl)pyrimidine-2,4-diamine

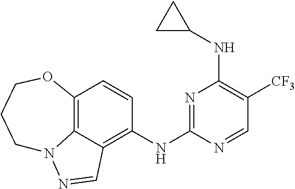 [SMILES: FC(F)(F)c1cnc(Nc2ccc3c4c2cnn4CCCO3)nc1NC1CC1]

### Step 1: 4-bromo-1H-indazol-7-ol

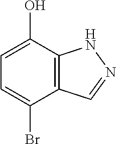 [SMILES: Oc1ccc(Br)c2cn[nH]c12]

A mixture of 4-bromo-7-methoxy-1H-indazole (800 mg, 3.52 mmol), BBr₃ (8.83 g, 35.2 mmol) in DCM (10 mL) was stirred at 25° C. for 16 hrs under N₂ atmosphere. The reaction mixture was quenched by aq. NaHCO₃ (30 mL) at 0° C. and extracted with EtOAc (50 mL×3). The combined organic layers were concentrated under reduced pressure to give 4-bromo-1H-indazol-7-ol (Intermediate 160-a) as a white solid (750 mg crude). LCMS (ESI) m/z 213.0, 215.0 [M+H]⁺.

### Step 2: 5-bromo-9-oxa-1,2-diazatricyclo[6.4.1.04,13]trideca-2,4,6,8(13)-tetraene

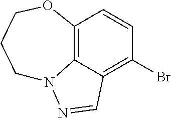 [SMILES: Brc1ccc2c3c1cnn3CCCO2]

A mixture of 4-bromo-1H-indazol-7-ol (750 mg, 3.52 mmol), 1-bromo-3-chloro-propane (609 mg, 3.87 mmol) and Cs₂CO₃ (3.44 g, 10.5 mmol) in DMF (1 mL) was stirred at 100° C. for 16 hrs. The reaction was quenched with water (2 mL) and extracted with EtOAc (5 mL×3). The combined organic layers were concentrated under reduced pressure and purified by flash silica gel chromatography (ISCO®; 12 g SepaFlash® Silica Flash Column, Eluent of 0˜50% Ethyl acetate/Petroleum ethergradient @ 25 mL/min) to provide 5-bromo-9-oxa-1,2-diazatricyclo[6.4.1.04,13]trideca-2,4,6,8(13)-tetraene (Intermediate 160-b) as a white solid (300 mg, 33.67% yield). ¹H NMR (400 MHz, DMSO-d₆) δ ppm 2.34-2.42 (m, 2H), 4.34-4.41 (m, 2H), 4.47-4.52 (m, 2H), 6.81 (d, J=8.07 Hz, 1H), 7.21 (d, J=8.07 Hz, 1H), 8.06 (s, 1H); LCMS (ESI) m/z 253.1, 255.1 [M+H]⁺.

### Step 3: N-(9-oxa-1,2-diazatricyclo[6.4.1.04,13]trideca-2,4,6,8(13)-tetraen-5-yl)-1,1-diphenyl-methanimine

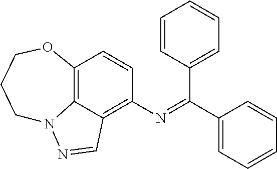 [SMILES: c1ccc(C(=Nc2ccc3c4c2cnn4CCCO3)c2ccccc2)cc1]

A mixture of 5-bromo-9-oxa-1,2-diazatricyclo[6.4.1.04,13]trideca-2,4,6,8(13)-tetraene (Intermediate 160-b) (300 mg, 1.19 mmol), diphenylmethanimine (236 mg, 1.30 mmol), XantPhos Pd G3 (112 mg, 118 mmol) and Cs₂CO₃ (1.16 g, 3.56 mmol) in dioxane (5 mL) was degassed and purged with N₂ for 3 times, then the mixture was stirred at 90° C. for 16 hrs under N₂ atmosphere. The solvent was evaporated and the residue was purified by flash silica gel chromatography (ISCO®; 12 g SepaFlash® Silica Flash Column, Eluent of 0˜50% Ethyl acetate/Petroleum ethergradient @ 20 mL/min) to provide N-(9-oxa-1,2-diazatricyclo[6.4.1.04,13]trideca-2,4,6,8(13)-tetraen-5-yl)-1,1-diphenyl-methanimine (Intermediate 160-c) as a white solid (200 mg, 47.74% yield). LCMS (ESI) m/z 354.2 [M+H]⁺.

### Step 4: 9-oxa-1,2-diazatricyclo[64.1.04,13] trideca-2,4,6,8(13)-tetraen-5-amine

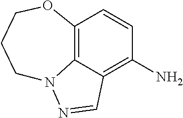 [SMILES: Nc1ccc2c3c1cnn3CCCO2]

A mixture of N-(9-oxa-1,2-diazatricyclo[6.4.1.04,13]trideca-2,4,6,8(13)-tetraen-5-yl)-1,1-diphenyl-methanimine (Intermediate 160-c) (200 mg, 565 mmol), NaOAc (116 mg, 1.41 mmol) and NH₂OH·HCl (78.6 mg, 1.13 mmol) in MeOH (10 mL) was stirred at 25° C. for 1 hr. The reaction was purified by flash silica gel chromatography (ISCO®; 4 g SepaFlash® Silica Flash Column, Eluent of 0˜100% Ethyl acetate/Petroleum ethergradient @ 12 mL/min) to provide 9-oxa-1,2-diazatricyclo[6.4.1.04,13]trideca-2,4,6,8(13)-tetraen-5-amine (Intermediate 160-d) as a white solid (80 mg, 74.71% yield). LCMS (ESI) m/z 190.0 [M+H]⁺.

### Step 5: N⁴-cyclopropyl-N²-(9-oxa-1,2-diazatricyclo [6.4.1.04,13]trideca-2,4,6,8(13)-tetraen-5-yl)-5-(trifluoromethyl)pyrimidine-2,4-diamine

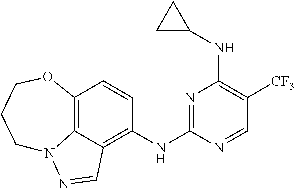 [SMILES: FC(F)(F)c1cnc(Nc2ccc3c4c2cnn4CCCO3)nc1NC1CC1]

A mixture of 9-oxa-1,2-diazatricyclo[6.4.1.04,13]trideca-2,4,6,8(13)-tetraen-5-amine (Intermediate 160-d) (40 mg, 211 mmol), 2-chloro-N-cyclopropyl-5-(trifluoromethyl)pyrimidin-4-amine (Intermediate 68-a) (50.2 mg, 211 mmol) and HCl (1 M, 211 mL) in i-PrOH (2 mL) was stirred at 80° C. for 1 hr. i-PrOH was evaporated. The crude product was triturated with EtOAc (0.5 mL) to provide the title compound as a white solid (53.5 mg, 64.83% yield). ¹H NMR (400 MHz, CD₃OD) δ ppm 0.68-0.87 (m, 4H), 2.44-2.51 (m, 2H), 2.91 (br s, 1H), 4.36-4.44 (m, 2H), 4.51-4.56 (m, 2H), 6.97 (d, J=8.14 Hz, 1H), 7.44 (br s, 1H), 7.96-8.27 (m, 2H): LCMS (ESI) m/z 391.1 [M+H]⁺.

### Example 161

### N₄-ethyl-N²-(9-oxa-1,2-diazatriyclo[6.4.1.04,13]trideca-2,4,6,8(13)-tetraen-5-yl)-5-(trifluoromethyl)pyrimidine-2,4-diamine

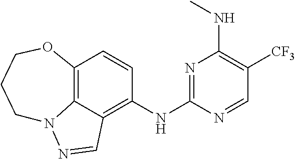 [SMILES: CNc1nc(Nc2ccc3c4c2cnn4CCCO3)ncc1C(F)(F)F]

A mixture of 9-oxa-1,2-diazatricyclo[6.4.1.04,13]trideca-2,4,6,8(13)-tetraen-5-amine (Intermediate 160-d) (40 ng, 0.211 mmol), 2-chloro-N-methyl-5-(trifluoromethyl)pyrimidin-4-amine (Intermediate 1-a) (44.7 ng, 211 mmol) and HCl (1 M, 211 mL) in i-PrOH (2 mL) was stirred at 80° C. for 1 hr under N₂ atmosphere. i-PrOH was evaporated and the crude product was triturated with EtOAc (0.5 mL) to provide the title compound as a white solid (61.5 mug, 79.85% yield). ¹H NMR (400 MHz, CD₃OD) δ ppm 2.37-2.56 (m, 2H), 2.94 (s, 3H), 4.38-4.44 (m, 2H), 4.51-4.57 (m, 2H), 6.98 (d, J=7.92 Hz, 1H), 7.27 (br d, J=7.92 Hz, 1H), 7.93-8.27 (m, 2H); LCMS (ESI) m/z 365.1 [M+H]⁺.

### Example 162

### 2-[4-[(5-chloro-4-ethoxy-7H-pyrrolo[2,3-d]pyrimidin-2-yl)amino]indazol-1-yl]-2-methyl-propanenitrile

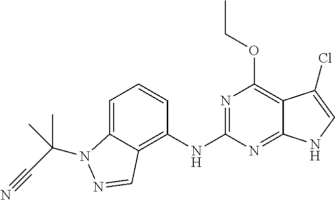 [SMILES: CCOc1nc(Nc2cccc3c2cnn3C(C)(C)C#N)nc2[nH]cc(Cl)c12]

### Step 1: 2-[4-[[5-chloro-4-ethoxy-7-(2-trimethylsilylethoxymethyl)pyrrolo[2,3-d]pyrimidin-2-yl]amino]indazol-1-yl]-2-methyl-propanenitrile

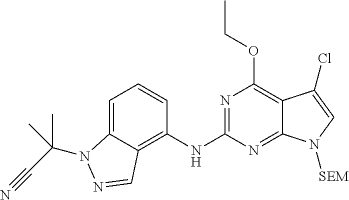 [SMILES: CCOc1nc(Nc2cccc3c2cnn3C(C)(C)C#N)nc2c1c(Cl)cn2COCC[Si](C)(C)C]

To a solution of 2-(4-aminoindazol-1-yl)-2-methyl-propanenitrile (Intermediate 31-d) (107 mg, 538 μmol) and 2-[(2,5-dichloro-4-ethoxy-pyrrolo[2,3-d]pyrimidin-7-yl)methoxy]ethyl-trimethyl-silane (Intermediate 158-c) (150 mg, 413 μmol) in toluene (4 mL) was added Pd₂(dba)₃ (37.9 mg, 41.4 μmol), Xantphos (47.9 mg, 82.8 μmol) and Cs₂CO₃ (337 mg, 1.03 mmol). The mixture was stirred at 100° C. for 16 hrs under N₂ atmosphere and then filtered. The filtrate was concentrated to give a crude product, which was purified by flash chromatography (ISCO®; 4 g SepaFlash® Silica Flash Column, petroleum ether/EtOAc with EtOAc from 0-50%, 12 mL/Min, 254 mn) to give 2-[4-[[5-chloro-4-ethoxy-7-(2-trimethylsilylethoxymethyl) pyrrolo[2,3-d]pyrimidin-2-yl]amino]indazol-1-yl]-2-methyl-propanenitrile (Intermediate 162-a) as a brown solid (150 mg, 68.9% yield). ¹H NMR (400 MHz, DMSO-d₆) δ ppm −0.14 (s, 9H), 0.85 (t, J=8.1 Hz, 2H), 1.42 (t, J=7.0 Hz, 3H), 2.09 (s, 6H), 3.53 (t, J=8.1 Hz, 2H), 4.59 (q, J=7.1 Hz, 2H), 5.47 (s, 2H), 7.39 (s, 1H), 7.44 (d, J=4.9 Hz, 2H), 8.01-8.27 (m, 1H), 8.62 (s, 1H), 9.67 (s, 1H); LCMS (ESI) m/z 526.3 [M+H].

### Step 2: 2-[4-[[5-chloro-4-ethoxy-7(hydroxymethyl)pyrrolo[2,3-d]pyrimidin-2-yl]amino]indazol-1-yl]-2-methyl-propanenitrile

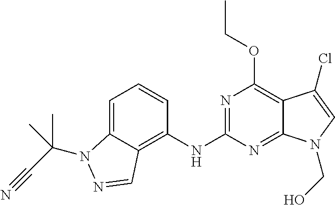 [SMILES: CCOc1nc(Nc2cccc3c2cnn3C(C)(C)C#N)nc2c1c(Cl)cn2CO]

A mixture of 2-[4-[[5-chloro-4-ethoxy-7-(2-trimethylsilylethoxymethyl)pyrrolo[2,3-d]pyrimidin-2-yl]amino]indazol-1-yl]-2-methyl-propanenitrile (Intermediate 162-a) (140 mg, 266 mmol) in TFA (1 mL) and DCM (2 mL) was stirred at 30° C. for 1 hr under N₂ atmosphere. The solution was concentrated to give 2-[4-[[5-chloro-4-ethoxy-7 (hydroxymethyl)pyrrolo[2,3-d]pyrimidin-2-yl]amino]indazol-1-yl]-2-methyl-propanenitrile (Intermediate 162-b) as a yellow oil (110 mg, crude), which was used for next step directly without further purification. LCMS (ESI) m/z 426.3 [M+H]⁺.

### Step 3: 2-[4-[(5-chloro-4-ethoxy-7H-pyrrolo[2,3-d]pyrimidin-2-yl)amino]indazol-1-yl]-2-methyl-propanenitrile

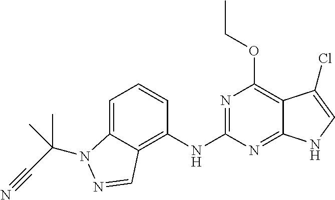 [SMILES: CCOc1nc(Nc2cccc3c2cnn3C(C)(C)C#N)nc2[nH]cc(Cl)c12]

A solution of 2-[4-[[5-chloro-4-ethoxy-7-(hydroxymethyl)pyrrolo[2,3-d]pyrimidin-2-yl]amino]indazol-1-yl]-2-methyl-propanenitrile (Intermediate 162-b) (110 mg, 258 μmol) and LiOH (18.5 mg, 774 μmol) in THF/H₂O (5:1, 6 mL) was stirred at 25° C. for 1 hr under N₂ atmosphere. The reaction was purified by Prep-HPLC (column: C18-1 150×30 mm×5 μm; mobile phase: [water (NH₃H₂O+NH₄HCO₃)-ACN]; B %: 50%-90%, 9 min) to provide the title compound as a brown solid (22.5 mg, 22.0% yield). ¹H NMR (400 MHz, DMSO-d₆) δ ppm 1.55 (t, J=7.1 Hz, 3H), 2.15 (s, 6H), 4.71 (q, J=7.1 Hz, 2H), 7.31 (s, 1H), 7.49-7.61 (m, 2H), 8.22 (dd, J=6.6, 1.6 Hz, 1H), 8.73 (s, 1H), 9.63 (s, 1H), 11.87 (br s, 1H); LCMS (ESI) m/z 396.1 [M+H]⁺.

### Example 163

### N⁴-cyclobutyl-N²-(1-(2-(methylsulfonyl)ethyl)-1H-indazol-4-yl)-5-(trifluoromethyl)pyrimidine-2,4-diamine

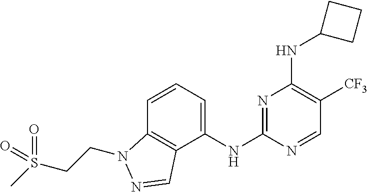 [SMILES: CS(=O)(=O)CCn1ncc2c(Nc3ncc(C(F)(F)F)c(NC4CCC4)n3)cccc21]

To a solution of 1-(2-methylsulfonylethyl)indazol-4-amine (Intermediate 139-b) (100 mg, 0.41 mmol, 1 eq) in IPA (1 mL) was added 2-chloro-N-cyclobutyl-5-(trifluoromethyl)pyrimidin-4-amine (Intermediate 150-a) (105 mg, 0.41 mmol, 1 eq) and HCl (1 M, 0.62 mL, 1.5 eq). The mixture was degassed and purged with N₂ for three times, then stirred at 80° C. for 4 hours under N₂ atmosphere. The mixture was basified at 0° C. by aq. NaHCO₃ to pH=7-8 and extracted with EtOAc (10 mL×3). Then the combined organic layers were washed with brine (10 mL), dried by Na₂SO₄, filtered and concentrated under reduced pressure to give a residue. The residue was purified by flash chromatography (ISCO®; 12 g SepaFlash® Silica Flash Column, petroleum ether/EtOAc with EtOAc from 0˜60%, 50 mL/min 254 mn/I₂/KMnO₄/PMA) to provide the title compound as a white solid (41.23 mg, 21.1%). ¹H NMR (400 MHz, DMSO-d₆) δ ppm 1.62-1.71 (m, 2H), 2.15-2.26 (m, 4H), 2.91 (s, 3H), 3.74 (t, J=6.78 Hz, 2H), 4.60-4.71 (m, 1H), 4.79 (t, J=6.90 Hz, 2H), 7.07 (br d, J=6.78 Hz, 1H), 7.31-7.40 (m, 2H), 7.88 (d, J=7.03 Hz, 1H), 8.25 (s, 1H), 8.46 (s, 1H), 9.81 (s, 1H); ¹⁹F NMR (377 MHz, DMSO-d₆) δ ppm −59.93 (s, 3F); LCMS (ESI) m/z 455.5 [M+H]⁺.

### Example 164

### 1-[4-[[4-(ethylamino)-5-(trifluoromethyl) pyrimidin-2-yl]amino]indazol-1-yl]-2-methyl-propan-2-ol

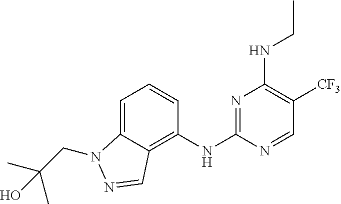 [SMILES: CCNc1nc(Nc2cccc3c2cnn3CC(C)(C)O)ncc1C(F)(F)F]

To a solution of 1-(4-aminoindazol-1-yl)-2-methyl-propan-2-ol (Intermediate 97-b) (200 mg, 974 mmol) and 2-chloro-N-ethyl-5-(trifluoromethyl)pyrimidin-4-amine (intermediate 69-a) (219 mg, 974 mmol) in IPA (5 mL) was added 2 drop 1M HCl. The mixture was stirred for 1 hr. and then purified by flash chromatography (ISCO®; 4 g SepaFlash® Silica Flash Column, petroleum ether/EtOAc with EtOAc from 0˜30%, 12 mL/min, 254 mn) to provide the title compound as a white solid (101.0 mg, 26.3% yield). ¹H NMR (400 MHz, DMSO-d₆) δ ppm 1.11-1.17 (m, 9H), 3.44-3.53 (m, 2H), 4.27 (s, 2H), 4.66 (s, 1H), 7.18 (br t, J=5.3 Hz, 1H), 7.25-7.35 (m, 2H), 7.82 (d, J=7.1 Hz, 1H), 8.22 (s, 1H), 8.39 (s, 1H), 9.73 (s, 1H), LCMS (ESI) m/z 395.1 [M+H]⁺.

### Example 165

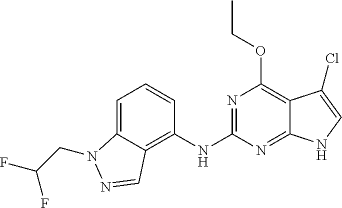 [SMILES: CCOc1nc(Nc2cccc3c2cnn3CC(F)F)nc2[nH]cc(Cl)c12]

### 5-chloro-N-[1-(2,2-difluoroethyl)indazol-4-yl]-4-ethoxy-7H-pyrrolo[2,3-d]pyrimidin-2-amine

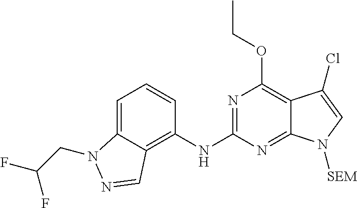 [SMILES: CCOc1nc(Nc2cccc3c2cnn3CC(F)F)nc2c1c(Cl)cn2COCC[Si](C)(C)C]

To a solution of 1-(2,2-difluoroethyl))indazol-4-amine (Intermediate 94-b) (106 mg, 538 mmol and 2-[(2,5-dichloro-4-ethoxy-pyrrolo[2,3-d]pyrimidin-7-yl)methoxy]ethyl-trimethyl-silane (Intermediate 158-c) (150 mg, 414 mol) in toluene (2 mL) was added d2(dba) (37.9 mg, 41.4 μmol), Xantphos (47.9 mg, 82.8 μmol), Cs₂CO₃ (337 mg, 1.03 mmol). The mixture was stirred at 100° C. for 16 hrs under N₂ atmosphere, then concentrated to give a crude product, which was purified by flash chromatography (ISCO® 4 g SepaFlash® Silica Flash Column, petroleum ether/EtOAc with EtOAc from 0˜50%, 12 mL/min, 254 mn) to give 5-chloro-N-[1-(2,2-difluoroethyl)indazol-4-yl]-4-ethoxy-7-(2-trimethylsilylethoxymethyl)pyrrolo[2,3-d]pyrimidin-2-amine (Intermediate 165-a) as a brown solid (150 trig, 69.3% yield). ¹H NMR (400 MHz, CD₃OD) δ ppm 0.00 (s, 9H), 1.00-1.04 (m, 2H), 1.59-1.61 (m, 3H), 3.63-3.77 (m, 2H), 4.71-4.77 (m, 3H), 4.87-4.97 (m, 2H), 5.62 (s, 2H), 6.18-6.56 (m, 1H), 7.17 (s, 1H), 7.35 (d, J=8.3 Hz, 1H), 7.55 (t, J=8.1 Hz, 1H), 8.23 (d, J=7.8 Hz 1H), 8.51 (s, 1H).

### Step 2: [5-chloro-2-[[1-(2,2-difluoroethyl)indazol-4-yl]amino]-4-ethoxy-pyrrolo[2,3-d]pyrimidin-7-yl]methanol

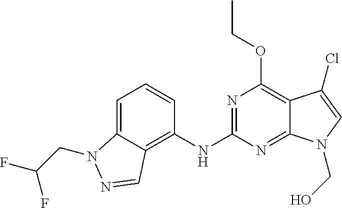 [SMILES: CCOc1nc(Nc2cccc3c2cnn3CC(F)F)nc2c1c(Cl)cn2CO]

A solution of 5-chloro-N-[1-(2,2-difluoroethyl)indazol-4-yl]-4-ethoxy-7-(2-trimethylsilylethoxy methyl)pyrrolo[2,3-d]pyrimidin-2-amine (Intermediate 165-a) (140 mg, 267 μmol) and TFA (1 mL) in DCM (2 mL) was stirred at 25° C. for 2 hrs. The resulting mixture was concentrated to give a [5-chloro-2-[[1-(2,2-difluoroethyl)indazol-4-yl]amino]-4-ethoxy-pyrrolo[2,3-d]pyrimidin-7-yl]methanol (Intermediate 165-b) as a yellow oil (110 mg, 97.2% yield). LCMS (ESI) m/z 423.2 [M+H]⁺.

### Step 3: 5-chloro-N-[1-(2,2-difluoroethyl)indazol-4-yl]-4-ethoxy-7H-pyrrolo[2,3-d]pyrimidin-2-amine

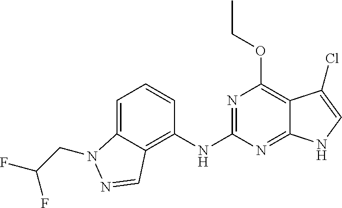 [SMILES: CCOc1nc(Nc2cccc3c2cnn3CC(F)F)nc2[nH]cc(Cl)c12]

A solution of [5-chloro-2-[[1-(2,2-difluoroethyl)indazol-4-yl]amino]-4-ethoxy-pyrrolo[2,3-d]pyrimidin-7-yl]methanol (Intermediate 165-b) (110 ng, 260 μmol) and LiOH (62.3 mg, 2.60 mmol) in THF/H₂O (5:1, 5 mL) was stirred at 25° C. for 1 hr. The reaction was purified by Pre-HPLC (column: C18 (250×50 mm×10 μM); mobile phase: [water(NH₃H₂O+NH₄HCO₃)-ACN]; B %: 35%-75%, 9 min) to provide the title compound as a brown solid (55.7 mg, 54.5% yield). ¹H NMR (400 MHz, DMSO-d₆) δ ppm 1.41 (br t, J=6.5 Hz, 3H), 4.57 (br d, J=6.6 Hz, 2H), 4.88 (br t, J=13.8 Hz, 2H), 6.20-6.63 (m, 1H), 7.16 (br s, 1H), 7.22-7.48 (m, 2H), 8.00 (br d, J=6.9 Hz, 1H), 8.53 (br s, 1H), 9.41 (br s, 1H), 11.72 (br s, 1H); LCMS (ESI) m/z 393.0 [M+H]⁺.

### Examples 166 and 167

### (R)-3-(4-((4-(cyclopropylamino)-5-(trifluoromethyl)pyrimidin-2-yl)amino)-1H-indazol-1-yl)-3-methyldihydrofuran-2(3H)-one (Example 166) and (S)-3-(4-((4-(cyclopropylamino)-5-(trifluoromethyl)pyrimidin-2-yl)amino)-1H-indazol-1-yl)-3-methyldihydrofuran-2(3H)-one (Example 167)

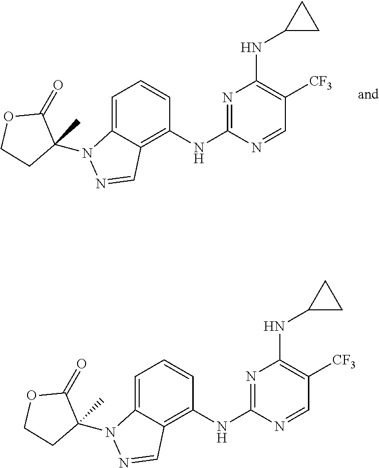 [SMILES: C[C@@]1(n2ncc3c(Nc4ncc(C(F)(F)F)c(NC5CC5)n4)cccc32)CCOC1=O ; C[C@]1(n2ncc3c(Nc4ncc(C(F)(F)F)c(NC5CC5)n4)cccc32)CCOC1=O]

### Step 1: 3-(4-nitro-1H-indazol-1-yl)dihydrofuran-2(3H)-one

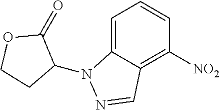 [SMILES: O=C1OCCC1n1ncc2c([N+](=O)[O-])cccc21]

To a solution of 4-nitro-H-indazole (7.91 g, 48.5 mmol) in DMF (150 mL) was added Cs₂CO₃ (22.1 g, 67.8 mmol) under N₂ atmosphere. After 30 min 3-bromotetrahydrofuran-2-one (8 g, 48.4 mmol) was added dropwise. The resulting mixture was stirred at 80° C. for 16 hrs under N₂ atmosphere. The reaction was quenched by H₂O (450 mL), extracted with EtOAc (500 mL×2), washed with aq. LiCl (500 mL×2) and brine (500 mL×2) to provide a crude product. The crude was purified by flash chromatography (ISCO®; 220 g SepaFlash® Q Silica Flash Column, petroleum ether/EtOAc with EtOAc from 0˜30%, 100 mL/min, 254 nm) to give 3-(4-nitroindazol-1-yl) tetrahydrofuran-2-one (Intermediate 166-a) as a gray solid (3.2 g, 26.7% yield). ¹H NMR (400 MHz, DMSO-d₆) δ ppm 2.83-3.02 (m, 2H)) 4.49 (td, J=9.3, 6.8 Hz, 1H), 4.68 (td, J=8.6, 2.8 Hz, 1H), 6.25 (t, J=9.7 Hz, 1H), 7.74 (t, J=8.1 Hz, 1H), 8.25 (dd, J=16.3, 8.1 Hz, 2H), 8.65 (s, 1H); LCMS (ESI) m/z 248.2 [M+H]⁺.

### Step 2: 3-methyl-3-(4-nitro-1H-indazol-1-yl)dihydrofuran-2(3H)-one

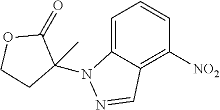 [SMILES: CC1(n2ncc3c([N+](=O)[O-])cccc32)CCOC1=O]

To a solution of 3-(4-nitroindazol-1-yl)tetrahydrofuran-2-one (Intermediate 166-a) (1 g, 4.05 mmol) in THF (20 mL) at −70° C. under N₂ atmosphere was added 2M LDA (4.05 mL, 8.09 mmol) dropwise. After addition, the mixture was stirred at this temperature for 30 min, and then CH₃I (1.72 g, 12.1 mmol) was added dropwise. The resulting mixture was stirred at −70° C. for another 2 hrs under N₂ atmosphere. The solution was warmed to 0° C., quenched with aq. NH₄Cl (20 mL), diluted with H₂O (50 mL), extracted with EtOAc (50 mL×2), washed with aq.LiCl (80 mL×2) and brine (100 mL×2) to give a crude product. The crude was purified by flash chromatography (ISCO®; 20 g SepaFlash® Silica Flash Column, petroleum ether/EtOAc with EtOAc from 0˜50%, 20 mL/min, 254 nm) to give 3-methyl-3-(4-nitroindazol-1-yl)tetrahydrofuran-2-one (Intermediate 166-b) as a yellow solid (700 mg, 22.1% yield) ¹H NMR (400 MHz, DMSO-d₆) δ ppm 2.04 (s, 3H), 2.64-2.76 (m, 1H), 2.99-3.15 (m, 1H), 4.38-4.61 (m, 2H), 7.70 (t, J=8.1 Hz, 1H), 8.17-8.28 (m, 2H), 8.58-8.70 (m, 1H); LCMS (ESI) m/z 262.2 [M+H]⁺.

### Step 3: 3-(4-amino-1H-indazol-1-yl)-3-methyldihydrofuran-2(3H)-one

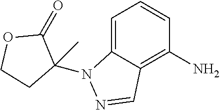 [SMILES: CC1(n2ncc3c(N)cccc32)CCOC1=O]

To a solution of 3-methyl-3-(4-nitroindazol-1-yl)tetrahydrofuran-2-one (Intermediate 166-b) (700 mg, 2.68 mmol) in THF (20 mL) was added Pd/C (284 mg, 10 wt % Pd with 50 wt % water) under N₂ atmosphere. The suspension was degassed and purged with H₂ for 3 times. The mixture was stirred under H₂ (15 psi) at 25° C. for 4 hrs. The solution was filtered and concentrated to give the crude 3-(4-aminoindazol-1-yl)-3-methyl-tetrahydrofuran-2-one (Intermediate 166-c) as a yellow solid (400 mg, 64.5% yield), ¹H NMR (400 MHz, DMSO-d₆) δ ppm 1.93 (s, 3H), 2.53-2.56 (m, 1H), 2.85-2.97 (m, 1H), 4.30-4.54 (m, 2H), 5.88 (s, 2H), 6.21 (d, J=7.5 Hz, 1H), 6.59 (d, J=8.4 Hz, 1H), 7.05 (t, J=7.9 Hz, 1H), 8.15 (d, J=0.9 Hz, 1H); LCMS (ESI) m/z 232.2 [M+H]⁺.

### Step 4: 3-(4-((4-(cyclopropylamino)-5-(trifluoromethyl)pyrimidin-2-yl)amino)-1H-indazol-1-yl)-3-methyldihydrofuran-2(3H)-one

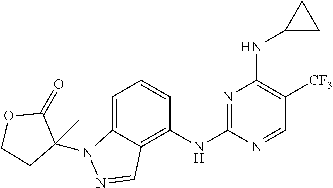 [SMILES: CC1(n2ncc3c(Nc4ncc(C(F)(F)F)c(NC5CC5)n4)cccc32)CCOC1=O]

A mixture of 3-(4-aminoindazol-1-yl)-3-methyl-tetrahydrofuran-2-one (Intermediate 166-c) (100 mg, 0.432 mmol), 2-chloro-N-cyclopropyl-5-(trifluoromethyl)pyrimidin-4-amine (Intermediate 68-a) (102 mg, 0.432 mmol) in TFE (5 mL) was added TFA (246 mg, 2.16 mmol), and then the mixture was stirred at 80° C. for 1 hr under N₂ atmosphere. The reaction solution was quenched by aq. NaHCO₃ (20 mL), extracted with EtOAc (20 mL×2), washed with brine (20 mL) and dried over Na₂SO₄ to give a residue. The residue was purified by flash chromatography (ISCO®; 4 g SepaFlash® Silica Flash Column, petroleum ether/EtOAc with EtOAc from 0˜50%, 12 mL/min, 254 nm) to give 3-(4-((4-(cyclopropylamino)-5-(trifluoromethyl)pyrimidin-2-yl)amino)-1H-indazol-1-yl)-3-methyldihydrofuran-2(3H)-one (Intermediate 166-d) as a white solid (108 mg, 51.5% yield). LCMS (ESI) m/z 433.3 [M+H]⁺.

### Step 5: (R)-3-(4-((4-(cyclopropylamino)-5-(trifluoromethyl)pyrimidin-2-yl)amino)-1H-indazol-1-yl)-3-methyldihydrofuran-2(3H)-one (Example 166) and (S)-3-(4-((4-(cyclopropylamino)-5-(trifluoromethyl)pyrimidin-2-yl)amino)-11H-indazol-1-yl)-3-methyldihydrofuran-2(3H)-one (Example 167)

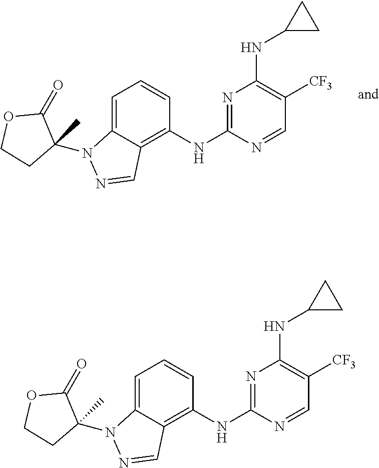 [SMILES: C[C@@]1(n2ncc3c(Nc4ncc(C(F)(F)F)c(NC5CC5)n4)cccc32)CCOC1=O ; C[C@]1(n2ncc3c(Nc4ncc(C(F)(F)F)c(NC5CC5)n4)cccc32)CCOC1=O]

The racemate was purified by SFC (i-PrOH(0.1% NH₃H₂O)/75% CO₂ on DAICEL CHIRALPAK IG (250 mm×50 mm×10 μm) at 200 mL/min FR; BRP=100 bar) to provide the two fractions, which after removal of solvents, provided the following two products respectively: Fraction A (33.7 mg, RT=5.648 min, @ (Column: ChiralPak IG-3 100×4.6 mm I.D., 3 μm Mobile phase: A: CO₂ B: i-PrOH (0.05% DEA) Gradient: from 5% to 40% of B in 4 min and hold 40% for 3 min, then 5% of B for 1 min Flow rate: 2.5 mL/min. Column temp.: 40° C. ABPR: 100 bar) was tentatively assigned as compound Example 166. ¹H NMR (400 MHz, DMSO-d₆) δ ppm 0.60-0.85 (m, 4H), 1.97 (s, 3H), 2.54-2.65 (n, 1H), 2.87 (br dd, J=6.9, 3.6 Hz, 1H), 2.94-3.05 (m, 1H), 4.32-4.55 (m, 2H), 7.17 (d, J=8.4 Hz, 1H), 7.25 (br s, 1H), 7.36 (t, J=8.1 Hz, 1H), 8.22 (s, 2H), 8.57 (s, 1H), 9.94 (s, 1H); LCMS (ESI) m/z 433.3 [M+H]⁺.

Fraction B (23.6 ng, RT=6.262 min, @(Column: ChiralPak IG-3 100×4.6 mm I.D., 3 μm Mobile phase: A: CO₂ B: i-PrOH (0.05% DEA) Gradient: from 5% to 40% of B in 4 min and hold 40% for 3 min, then 5% of B for 1 mi Flow rate: 2.5 mL/min. Column temp.: 40° C. BPR: 100 bar) was tentatively assigned as compound Example 167. ¹H NMR (400 MHz, DMSO-d₆) δ ppm 0.65-0.83 (m, 4H), 1.97 (s, 3H), 2.54-2.64 (m, 1H), 2.87 (dt, J=6.8, 3.4 Hz, 1H), 2.95-3.04 (m, 1H), 4.36-4.55 (m, 2H), 7.17 (d, J=8.6 Hz, 1H), 7.26 (br s, 1H), 7.36 (t, J=8.0 Hz, 1H), 8.19 (s, 2H), 8.57 (s, 1H), 9.94 (s, 1H); LCMS (ESI) in/z 433.3 [M+H]⁺.

### Example 168

### N⁴-cyclopropyl-N²-(1-(cyclopropylmethyl)-1H-indazol-4-yl)-5-(trifluoromethyl)pyrimidine-2,4-diamine

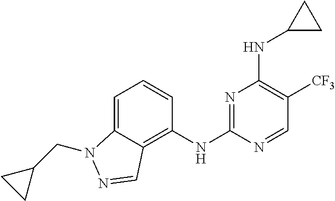 [SMILES: FC(F)(F)c1cnc(Nc2cccc3c2cnn3CC2CC2)nc1NC1CC1]

### Step 1: 1-(cyclopropylmethyl)-4-nitro-1H-indazole

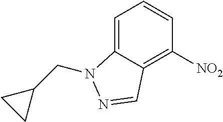 [SMILES: O=[N+]([O-])c1cccc2c1cnn2CC1CC1]

To a solution of 4-nitro-1H-indazole (1 g, 6.13 mmol) was added bromomethylcyclopropane (1.66 g, 12.3 mmol) and NaH (490 mg, 12.3 mmol, 60% in mineral oil) in DMF (10 mL). The mixture was stirred at 25° C. for 6 hrs under N₂ atmosphere. The reaction was quenched by H₂O (2 mL) and the resulting filtrate was concentrated under reduced pressure to give a crude product, which was purified by prep-HPLC (column: C18 (250×50 mm×10 □m); mobile phase: [water (NH₃H₂O+NH₄HCO₃)-ACN]; B %: 30%-70%, 9 min) to give 1-(cyclopropylmethyl)-4-nitro-1H-indazole (Intermediate 168-a) as a yellow solid (450 mg, 33.8% N yield). ¹H NMR (400 MHz, DMSO-d₆) δ ppm 0.42-0.46 (m, 2H), 0.48-0.53 (m, 2H), 1.26-1.35 (m, 1H), 4.44-4.47 (m, 2H), 7.62-7.68 (m, 1H), 8.16-8.20 (m, 1H), 8.31-8.35 (m, 1H), 8.51-8.54 (m, 1H); LCMS (ESI) m/z 218.2 [M+H]⁺.

### Step 2: 1-(cyclopropylmethyl)-1H-indazol-4-amine

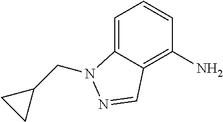 [SMILES: Nc1cccc2c1cnn2CC1CC1]

A mixture of 1-(cyclopropylmethyl)-4-nitro-indazole (Intermediate 168-a) (450 mg, 2.07 nmol) and Pd/C (220 mg, 10% purity) in MeOH (10 mL) was stirred at 25° C. for 2 hrs under N₂ atmosphere. The reaction mixture was filtered and the filtrate was concentrated under reduced pressure to provide 1-(cyclopropylmethyl)-1H-indazol-4-amine (Intermediate 168-b) as a black oil (250 mg, 64.5% yield). ¹H NMR (400 MHz, DMSO-d₆) δ ppm 0.32-0.37 (m, 2H), 0.46 (s, 2H), 1.19-1.28 (m, 1H), 4.11-4.19 (m, 2H), 5.64-5.86 (m, 2H), 6.07-6.25 (m, 1H), 6.66-6.72 (m, 1H), 6.97-7.06 (m, 1H), 8.02-8.09 (m, 1H); LCMS (ESI) m/z 188.2 [M+H]⁺.

### Step 3: N4-cyclopropyl-N²-(1-(cyclopropylmethyl)-1H-indazol-4-yl)-5-(trifluoromethyl) pyrimidine-2,4-diamine

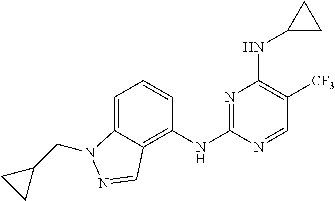 [SMILES: FC(F)(F)c1cnc(Nc2cccc3c2cnn3CC2CC2)nc1NC1CC1]

To a solution of 1-(cyclopropylmethyl)indazol-4-amine (Intermediate 168-b) (50 mg, 0.267 mmol) was added 2-chloro-N-cyclopropyl-5-(trifluoromethyl)pyrimidin-4-amine (Intermediate 68-a) (63.5 mg, 0.267 mmol) and 12 N HCl (a drop) in i-PrOH (2 mL). The mixture was stirred at 80° C. for 1 hr under N2 atmosphere. The resulting solution was concentrated under reduced pressure to give a crude product, which was triturated with EtOAc at 25° C. for 1 hr to give the title compound as a white solid (40 mg, 38.6% yield). ¹H NMR (400 MHz, DMSO-d₆) δ ppm 0.39 (br s, 2H) 0.43-0.51 (m, 2H), 0.71-0.77 (m, 2H), 0.78-0.85 (m, 2H), 1.21-1.32 (m, 1H), 2.88-2.96 (m, 1H), 4.27-4.33 (m, 2H), 7.40 (d, J=17.39 Hz, 2H), 7.91-8.23 (m, 2H), 8.46 (s, 2H), 10.67 (s, 1H); LCMS (ESI) m/z 389.1 [M+H]⁺.

### Example 169

### N4-cyclopropyl-N2-(1-isopropyl-1H-indazol-4-yl)-5-(trifluoromethyl)pyrimidine-2,4-diamine

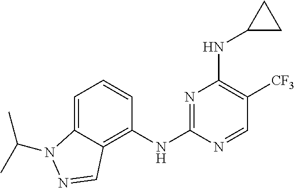 [SMILES: CC(C)n1ncc2c(Nc3ncc(C(F)(F)F)c(NC4CC4)n3)cccc21]

### Step 1: 1-isopropyl-4-nitro-1H-indazole

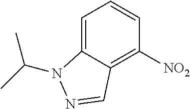

To a solution of 4-nitro-2H-indazole (500 mg, 3.06 mmol,) in DMF (20 mL) was added NaH (367 mg, 60% in mineral oil) at 0° C. After addition, the mixture was stirred at this temperature for 30 mins, and then 2-iodopropane (0.611 mL, 6.12 mmol) was added dropwise at 0° C. The resulting mixture was stirred at 25° C. for 2 hrs. The reaction mixture was quenched by 120 (100 mL) at 25° C., diluted with aq. LiCl (50 mL) and extracted with EtOAc (50 mL×3). The combined organic layers were washed with aq NaCl 120 mL (40 mL×3), dried over Na₂SO₄, filtered, and concentrated under reduced pressure. The residue was purified by column chromatography (SiO2, Petroleum ether/Ethyl acetate=0/0 to 3/1) to give 1-isopropyl-4-nitro-indazole (Intermediate 169-a) as a yellow solid (220 mg, 35.1% yield). ¹H NMR (400 MHz, DMSO-d₆) δ ppm 1.51 (d, J=6.60 Hz, 6H), 5.05-5.28 (m, 1H), 7.63 (t, J=8.03 Hz, 1H), 8.08-8.37 (m, 2H), 8.53 (s, 1H); LCMS (ESI) m/z 206.2 [M+H]⁺.

### Step 2: 1-isopropyl-1H-indazol-4-amine

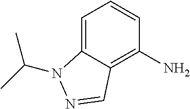

To a solution of 1-isopropyl-4-nitro-indazole (Intermediate 169-a) (220 mg, 1.07 mmol) in MeOH (20 mL) was added Pd/C (113 mg, 10 wt % Pd with 50 wt % water) under N₂ atmosphere. The suspension was degassed and purged with H₂ for 3 times. The mixture was stirred under H₂ (50 psi) at 25° C. for 16 hrs. The reaction mixture was filtered and the filtrate was concentrated under reduced pressure to give 1-isopropyl-1H-indazol-4-amine (Intermediate 169-b) as a white solid (180 mg, 95.8% yield). ¹H NMR (400 MHz, DMSO-d₆) δ ppm 1.41-1.46 (m, 6H), 4.78 (s, 1H), 5.73 (s, 2H), 6.13 (d, J=7.26 Hz, 1H), 6.69 (d, J=8.36 Hz, 1H), 7.00 (t, J=7.92 Hz, 1H), 8.07 (s, 1H).

### Step 3: N⁴-cyclopropyl-N²-(1-isopropyl-1H-indazol-4-yl)-5-(trifluoromethyl) pyrimidine-2,4-diamine

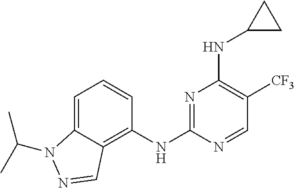 [SMILES: CC(C)n1ncc2c(Nc3ncc(C(F)(F)F)c(NC4CC4)n3)cccc21]

A mixture of 1-isopropylindazol-4-amine (Intermediate 169-b) (50 mg, 0.285 mmol), 2-chloro-N-cyclopropyl-5-(trifluoromethyl)pyrimidin-4-amine (Intermediate 68-a) (67.8 mg, 0.285 mmol), 1N HCl (285 mL, 0.285 mmol) in i-PrOH (2 mL) was stirred at 80° C. for 2 hrs under N₂ atmosphere. The reaction mixture was filtered and the filtrate was concentrated under reduced pressure to give a residue. The residue was purified by prep-HPLC (column: Boston Prime C18 150×30 mm×5 μm; mobile phase: [water (ammonia hydroxide v/v)-ACN]; B %: 46%-76%, 8 min) to give the title compound as a white solid (27.7 mg, 25.8% yield). ¹H NMR (400 MH, DMSO-d₆) δ ppm 0.53-0.87 (m, 4H), 1.46-1.47 (d, J=6.60 Hz, 6H), 2.718-2.93 (m, 1H), 4.82-5.01 (m, 1H), 7.21 (br s, 1H), 7.25-7.39 (m, 2H), 8.18 (br d, J=6.38 Hz, 1H), 8.47 (s, 1H), 9.84 (s, 1H); LCMS (ESI) m/z 377.2 [M+H]⁺.

### Example 170

### N⁴-cyclopropyl-N²-(1-(2-(cyclopropylsulfonyl)ethyl)-1H-indazol-4-yl)-5-(trifluoromethyl)pyrimidine-2,4-diamine

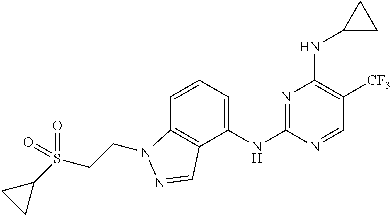 [SMILES: O=S(=O)(CCn1ncc2c(Nc3ncc(C(F)(F)F)c(NC4CC4)n3)cccc21)C1CC1]

### Step 1: 1-(2-chloroethyl)-4-nitro-1H-indazole

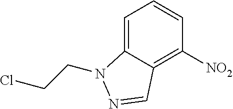 [SMILES: O=[N+]([O-])c1cccc2c1cnn2CCCl]

To a solution of 4-nitro-1H-indazole (5 g, 30.6 mmol) in DMF (400 ml) was added NaH (2.45 g, 61.3 mmol, 60% in mineral oil) under N₂ atmosphere. After 30 mins, 1-bromo-2-chloro-ethane (8.79 g, 61.3 mmol) was added dropwise. The resulting mixture was stirred at 20° C. for 16 hrs tinder N₂ atmosphere. The reaction was quenched by H₂O (1500 mL), extracted with EtOAc (1000 mL×2), washed with aq. LiCl (800 mL×2) and brine (800 mL×2) to give a crude product. The residue was purified by flash chromatography (ISCO®; 120 g SepaFlash® Silica Flash Column, petroleum ether/EtOAc with EtOAc from 0˜30%, 100 mL/min, 254 nm) to give 1-(2-chloroethyl)-4-nitro-1H-indazole (Intermediate 170-a) as a yellow solid (1 g, 14.5% yield). ¹H NMR (400 MHz, DMSO-d₆) δ ppm 4.14 (t, J=5.7 Hz, 2H), 4.93 (t, J=5.6 Hz, 2H), 7.59-7.74 (m, 1H), 8.16-8.23 (m, 1H), 8.35 (d, J=8.4 Hz, 1H), 8.60 (s, 1H).

### Step 2: 1-(2-(cyclopropylsulfonyl)ethyl)-4-nitro-1H-indazole

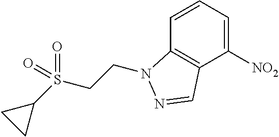 [SMILES: O=[N+]([O-])c1cccc2c1cnn2CCS(=O)(=O)C1CC1]

To a solution of 1-(2-chloroethyl)-4-nitro-indazole (Intermediate 170-a) (450 mg, 1.99 mmol) in DMF (20 mL) under N₂ atmosphere was added sodium cyclopropanesulfinate (511 mg, 3.99 nmol). The mixture was stirred at 60° C. for 2 hrs under N₂ atmosphere. The reaction solution was poured into H₂O (100 mL), extracted with EtOAc (100 mL×2), washed with aq. LiCl (80 mL×2) and brine (80 mL) to give a crude product, which was purified by flash chromatography (ISCO®; 20 g SepaFlash® Silica Flash Column, petroleum ether/EtOAc with EtOAc from 0˜50%, 20 mL/min, 254 nm) to give 1-(2-cyclopropylsulfonylethyl)-4-nitro-indazole (Intermediate 170-b) as a yellow solid (500 mg, crude). ¹H NMR (400 MHz, CDCl₃) δ ppm 0.89-0.99 (m, 2H), 1.11-1.21 (m, 2H), 1.92-2.06 (m, 1H), 3.80 (t, J=6.5 Hz, 2H), 4.93-5.04 (m, 2H), 7.54-7.66 (m, 1H), 7.91-8.01 (m, 1H), 8.13-8.30 (m, 1H), 8.66-8.81 (m, 1H).

### Step 3: 1-(2-(cyclopropylsulfonyl)ethyl)-1H-indazol-4-amine

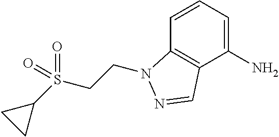 [SMILES: Nc1cccc2c1cnn2CCS(=O)(=O)C1CC1]

To a solution of 1-(2-cyclopropylsulfonylethyl)-4-nitro-indazole (Intermediate 170-b) (500 mg, 1.69 mmol) in MeOH (10 mL) was added Pd/C (284 mg, 10 wt % Pd with 50 wt % water) under N₂ atmosphere. The suspension was degassed and purged with H₂ for 3 times. The mixture was stirred under H₂ (30 Psi) at 25° C. for 16 hrs. The resulting mixture was filtered and the filtrate was concentrated to give a crude product. The crude product was purified by pre-HPLC (column: C18-1 150×30 mm×5 μm; mobile phase: [water (NH₃H₂O+NH₄HCO₃)-ACN]; B %: 0%-40%, 9 min) to give 1-(2-cyclopropylsulfonylethyl)indazol-4-amine (Intermediate 170-c) as a brown oil (80 mg, 17.8% yield). ¹H NMR (400 MHz, DMSO-d₁) δ ppm 0.84-1.01 (m, 4H), 2.54-2.61 (m, 1H), 3.65-3.82 (m, 2H), 4.68 (t, J=7.0 Hz, 2H), 5.82 (s, 2H), 6.18 (d, J=7.5 Hz, 1H), 6.71 (d, J=8.3 Hz, 1H), 7.06 (t, J=7.9 Hz, 1H), 8.13 (s, 1H).

### Step 4: N⁴-cyclopropyl-N²-(1-(2-(cyclopropylsulfonyl)ethyl)-1H-indazol-4-yl)-5-(trifluoromethyl)pyrimidine-2,4-diamine

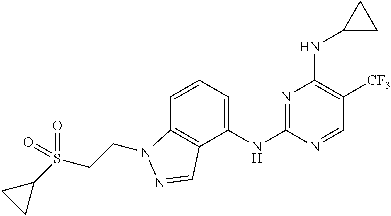 [SMILES: O=S(=O)(CCn1ncc2c(Nc3ncc(C(F)(F)F)c(NC4CC4)n3)cccc21)C1CC1]

A mixture of 1-(2-cyclopropylsulfonylethyl)indazol-4-amine (Intermediate 170-c) (80 mg, 0.180 mmol), 2-chloro-N-cyclopropyl-5-(trifluoromethyl)pyrimidin-4-amine (Intermediate 68-a) (42.9 mg, 0.180 mmol) and aq. HCl (1 M, 18.1 μL) in i-PrOH (2 mL) was stirred at 25° C. for 16 hrs under N₂ atmosphere. The resulting solution was concentrated to give a crude product. The crude was purified by pre-HPLC (column: C18 150×30 mm; mobile phase: [Water(HCl)-MeCN]; gradient: 16%-56% B over 9 min). The fractions were lyophilized to give the title compound as a white solid (20.7 mg, 24.5% yield). ¹H NMR (400 MHz, DMSO-d₆) δ (ppm 0.72-0.86 (m, 4H), 0.88-0.97 (m, 4H), 259 (br s, 1H), 2.91 (br s, 1H), 3.55-3.78 (m, 2H), 4.82-4.84 (m, 2H), 7.42-7.43 (br d, J=3.7 Hz, 2H), 8.01-8.23 (m, 2H), 8.46 (br s, 1H), 8.54 (s, 1H), 10.69 (br s, 1H); LCMS (ESI) m/z 489.1 [M+Na]⁺.

### Example 171

### N⁴-cyclopropyl-N²-(1-(2-(ethylsulfonyl)ethyl)-1H-indazol-4-yl)-5-(trifluoromethyl)pyrimidine-2,4-diamine

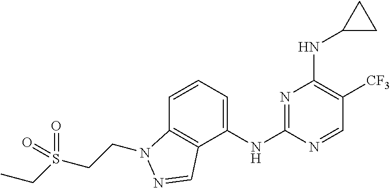 [SMILES: CCS(=O)(=O)CCn1ncc2c(Nc3ncc(C(F)(F)F)c(NC4CC4)n3)cccc21]

### Step 1: 1-(2-(ethylsulfonyl)ethyl)-4-nitro-1H-indazole

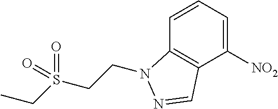 [SMILES: CCS(=O)(=O)CCn1ncc2c([N+](=O)[O-])cccc21]

To a solution of 1-(2-chloroethyl)-4-nitro-1H-indazole (Intermediate 170-a) (450 mg, 1.99 mmol) was stirred to dissolve in DMF (20 mL) was added sodium ethanesulfinate (462 mg, 3.98 mmol) dropwise. The mixture was stirred at 60° C. for 16 hrs under N₂ atmosphere. The reaction solution was poured into H₂O (100 mL), extracted with EtOAc (100 mL×2), washed with aq.LiCl (80 mL×2) and brine (80 mL×2) to give a crude product. The residue was purified by flash chromatography (ISCO®; 20 g SepaFlash® Silica Flash Column, petroleum ether/EtOAc with EtOAc from 0˜50%, 12 mL/min, 254 nm) to give 1-(2-ethylsulfonylethyl)-4-nitro-indazole (Intermediate 171-a) as a yellow solid (500 mg, 88.7% yield). ¹H NMR (400 MHz, CDCl₃) δ ppm 1.30 (t, J=7.5 Hz, 3H), 2.67 (q, J=7.5 Hz, 2H), 3.72 (t, J=6.4 Hz, 2H), 4.97 (t, J=6.3 Hz, 2H), 7.54-7.67 (m, 1H), 7.96 (d, J=8.4 Hz, 1H), 8.15-8.26 (m, 1H), 8.76 (s, 1H).

### Step 2: 1-(2-(ethylsulfonyl)ethyl)-4-nitro-1H-indazole

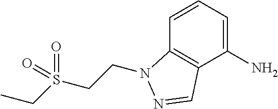 [SMILES: CCS(=O)(=O)CCn1ncc2c(N)cccc21]

To a solution of 1-(2-ethylsulfonylethyl)-4-nitro-indazole (Intermediate 171-a) (500 mg, 1.76 mmol) in MeOH (10 mL) was added Pd/C (186 mg, 10 wt % Pd with 50 wt % water) under N₂ atmosphere. The suspension was degassed and purged with H₂ for 3 times. The mixture was stirred under H₂ (15 psi) at 25° C. for 16 hrs. The suspension was filtered and the filtrate was concentrated to give a crude. The crude was purified by Pre-HPLC (column: C18 (250×50 mm×10 μm); mobile phase: [water (NH₃H₂O+NH₄HCO₃)-ACN]; B %: 0%-38%, 9 min) to give 1-(2-ethylsulfonylethyl) indazol-4-amine (Intermediate 171-b) as a yellow solid (290 mg, 65.1% yield). ¹H NMR (400 MHz, DMSO-d₆) δ ppm 1.12-1.18 (t, J=7.4 Hz, 3H), 2.95 (q, J=7.5 Hz, 2H), 3.65 (t, J=6.8 Hz, 2H), 4.66 (t, J=6.9 Hz, 2H), 5.77-5.95 (m, 2H), 6.18 (d, J=7.4 Hz, 1H), 6.71 (d, J=8.3 Hz, 1H), 7.07 (t, J=7.9 Hz, 1H), 8.15 (s, 1H).

### Step 3: N⁴-cyclopropyl-N²-(1-(2-(ethylsulfonyl)ethyl)-1H-indazol-4-yl)-5-(trifluoromethyl)pyrimidine-2,4-diamine

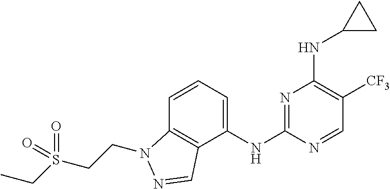 [SMILES: CCS(=O)(=O)CCn1ncc2c(Nc3ncc(C(F)(F)F)c(NC4CC4)n3)cccc21]

To a solution of 1-(2-ethylsulfonylethyl)indazol-4-amine (Intermediate 171-b) (50 mg, 0.197 mmol) and 2-chloro-N-cyclopropyl-5-(trifluoromethyl)pyrimidin-4-amine (Intermediate 68-a) (46.9 mg, 0.197 mmol) in i-PrOH (3 mL) was added HCl (1 M, 19.7 μL) under N₂ atmosphere. The mixture was stirred under N₂ at 80° C. for 1 hr. The resulting mixture was filtered and the filter cake was collected by filtration and triturated with EtOAc (10 mL) to give the title compound as a white solid (64.8 mg, 66.9% yield). ¹H NMR (400 MHz, DMSO-d₆) δ ppm 0.69-0.84 (m, 4H), 1.14 (t, J=7.4 Hz, 3H), 2.89 (br d, 3=3.3 Hz, 1H), 2.95-3.05 (m, 2H), 3.71 (t, J=6.68 Hz, 2H), 4.72-4.87 (m, 2H), 7.33-7.45 (m, 2H), 7.69 (br s, 1H), 8.17 (br d, J=6.8 Hz, 1H), 8.36 (s, 1H), 8.54 (s, 1H), 10.30 (br s, 1H); LCMS (ESI) m/z 455.1 [M+H]⁺.

### Example 172

### 1-(4-((4-(cyclopropylamino)-5-(trifluoromethyl)pyrimidin-2-yl)amino)-7-fluoro-1H-indazol-1-yl)-2-methylpropan-2-ol

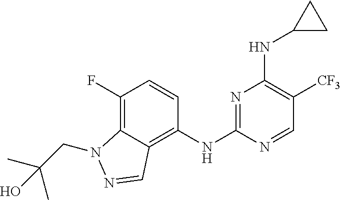 [SMILES: CC(C)(O)Cn1ncc2c(Nc3ncc(C(F)(F)F)c(NC4CC4)n3)ccc(F)c21]

### Step 1: 1-(4-bromo-7-fluoro-indazol-1-yl)-2-methyl-propan-2-ol

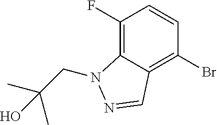 [SMILES: CC(C)(O)Cn1ncc2c(Br)ccc(F)c21]

A mixture of 4-bromo-7-fluoro-1H-indazole (2 g, 9.30 mmol), 2,2-dimethyloxirane (737 mg, 10.2 mmol), Cs₂CO₃ (4.24 g, 13.0 mmol) in DMF (10 mL) was stirred at 80° C. for 4 hrs and then concentrated under reduced pressure. The residue was purified by flash silica gel chromatography (ISCO®; 20 g SepaFlash® Silica Flash Column, Eluent of 0˜50% Ethyl acetate/Petroleum ether gradient @ 20 ml/min, 254 nm) to give 1-(4-bromo-7-fluoro-indazol-1-yl)-2-methyl-propan-2-ol (Intermediate 172-a) as a white solid (1.2 g, 44.9% yield). ¹H NMR (400 MHz, DMSO-d₆) δ ppm 1.04-1.18 (m, 6H), 4.39 (s, 2H) 4.63-4.73 (m, 1H), 7.18 (dd, J=11.88, 8.13 Hz, 1H), 7.31 (dd, J=8.07, 3.44 Hz, 1H), 8.12 (s, 1H); LCMS (ESI) m/z 287.0, 289.0 [M+H]⁺.

### Step 2: 1-[4-(benzhydrylideneamino)-7-fluoro-indazol-1-yl]-2-methyl-propan-2-ol

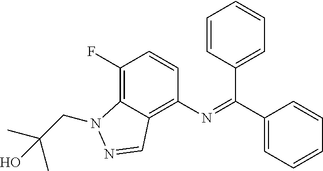 [SMILES: CC(C)(O)Cn1ncc2c(N=C(c3ccccc3)c3ccccc3)ccc(F)c21]

A mixture of 1-(4-bromo-7-fluoro-indazol-1-yl)-2-methyl-propan-2-ol (Intermediate 172-a) (1.2 g, 4.18 mmol), diphenylmethanimine (833 mg, 4.60 mmol), XantPhos Pd G₃ (396 mg, 0.417 mmol), Cs₂CO₃ (4.09 g, 12.5 mmol) in dioxane (10 mL) was degassed and purged with N₂ for 3 times. The mixture was stirred at 90° C. for 16 hrs under N₂ atmosphere, and then concentrated under reduced pressure. The residue was purified by flash silica gel chromatography (ISCO®; 20 g SepaFlash®) Silica Flash Column, Eluent of 0˜50% Ethyl acetate/Petroleum ethergradient @25 mL/min, 254 nm) to give 1-[4-(benzhydrylideneamino)-7-fluoro-indazol-1-yl]-2-methyl-propan-2-ol (Intermediate 172-b) as a white solid (1.2 g, 74.1% yield). ¹H NMR (400 MHz, DMSO-d₆) δ ppm 0.95-1.12 (m, 6H), 4.26-4.41 (m, 2H), 4.67 (s, 1H), 6.21 (dd, J=8.07, 3.18 Hz, 1H), 6.95 (dd, J=12.23, 807 Hz, 1H), 7.11-7.19 (m, 2H), 7.22-7.28 (m, 3H), 7.45-7.61 (m, 3H), 7.68-7.78 (m, 2H), 7.98 (d, J=2.08 Hz, 1H); LCMS (ESI) m/z 388.2 [M+H]⁺.

### Step 3: 1-(4-amino-7-fluoro-indazol-1-yl)-2-methyl-propan-2-ol

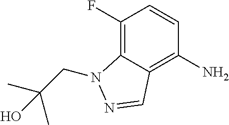 [SMILES: CC(C)(O)Cn1ncc2c(N)ccc(F)c21]

A mixture of 1-[4-(benzhydrylideneamino)-7-fluoro-indazol-1-yl]-2-methyl-propan-2-ol (Intermediate 172-b) (1.1 g, 2.84 mmol), N₂OH·HCl (394 mg, 5.68 mmol) and NaOAc (582 mg, 7.10 mmol) in MeOH (10 mL) was stirred at 25° C. for 1 hr. MeOH was evaporated, and the residue was purified by flash silica gel chromatography (ISCO®; 20 g SepaFlash® Silica Flash Column, Eluent of 0˜40% Ethyl acetate/Petroleum ether gradient @20 mL/min, 254 mn) to give 1-(4-amino-7-fluoro-indazol-1-yl)-2-methyl-propan-2-ol (Intermediate 172-c) as a white solid (600 mg, 94.6% yield). LCMS (ESI) m/z 224.3 [M+H]⁺.

### Step 4: 1-[4-[[4-(cyclopropylamino)-5-(trifluoromethyl)pyrimidin-2-yl]amino]-7-fluoro-indazol-1-yl]-2-methyl-propan-2-ol

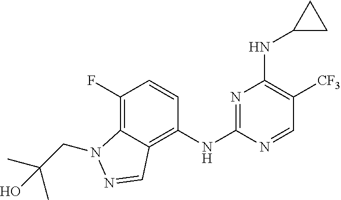 [SMILES: CC(C)(O)Cn1ncc2c(Nc3ncc(C(F)(F)F)c(NC4CC4)n3)ccc(F)c21]

A mixture of 1-(4-amino-7-fluoro-indazol-1-yl)-2-methyl-propan-2-ol (Intermediate 172-c) (50 mg, 0.223 mmol), 2-chloro-N-cyclopropyl-5-(trifluoromethyl)pyrimidin-4-amine (Intermediate 68-a) (53.2 mg, 0.223 mmol) and aq. HCl (1 M, 223 L) in i-PrOH (2 mL) was stirred at 80° C. for 1 hr. i-PrOH was evaporated, and the crude product was triturated with EtOAc at 25° C. for 1 hr; to provide the title compound as a white solid (60 mg, 63.1% yield). ¹H NMR (400 MHz, DMSO-d₆) δ ppm 0.59-0.88 (m, 4H), 1.10 (s, 6H), 2.87 (br d, J=3.08 Hz, 1H), 4.37 (s, 2H), 7.21 (dd, J=11.88, 8.58 Hz, 1H), 7.86-8.11 (m, 2H), 8.34-8.67 (m, 2H), 10.69 (br s, 1H); LCMS (ESI) m/z 425.1 [M+H]⁺.

### Example 173

### 1-[4-[[4-(cyclopropylamino)-5-(difluoromethyl)pyrimidin-2-yl]amino]indazol-1-yl]-2-methyl-propan-2-ol

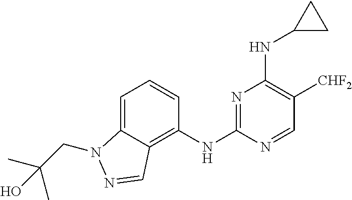 [SMILES: CC(C)(O)Cn1ncc2c(Nc3ncc(C(F)F)c(NC4CC4)n3)cccc21]

### Step 1: 2-chloro-4-(cyclopropylamino)pyrimidine-5-carbaldehyde

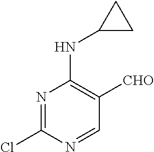 [SMILES: O=Cc1cnc(Cl)nc1NC1CC1]

A mixture of 2,4-dichloropyrimidine-S-carbaldehyde (4.1 g, 23.1 mmol) and cyclopropanamine (2.65 g, 46.4 nmol, 3.22 mL) in MeOH (20 mL) was stirred at 20° C. for 4 hours. The reaction mixture was concentrated under reduced pressure, diluted with water (50 mL) and extracted with EtOAc (50 mL×2). The combined organic layers were washed with brine (50 mL×2), dried over anhydrous Na₂SO₄, filtered and concentrated under reduced pressure. The crude product was purified by reverse HPLC (column: Xtimate C18 150×40 mm×5 μm; mobile phase: [water(NH₃H₂O+NH₄HCO₃)-ACN]; B %: 53%-68%, 18 min) to provide 2-chloro-4-(cyclopropylamino)pyrimidine-5-carbaldehyde (Intermediate 173-a) as a yellow solid (3.2 g, 71.1% yield). LCMS (ESI) m/z 198.1 [M+H]⁺.

### Step 2: 2-chloro-N-cyclopropyl-5-(difluoromethyl)pyrimidin-4-amine

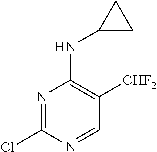 [SMILES: FC(F)c1cnc(Cl)nc1NC1CC1]

To a solution of 2-chloro-4-(cyclopropylamino)pyrimidine-5-carbaldehyde (Intermediate 173-a) (3.2 g, 16.2 mmol) in DCM (20 mL) was added DAST (6.5 g, 40.5 mmol). The reaction mixture was stirred at 20° C. for 4 hours, concentrated under reduced pressure, diluted with water (50 mL) and extracted with EtOAc (50 mL×2). The combined organic layers were washed with brine (50 mL×2), dried over anhydrous Na₂SO₄, filtered and concentrated under reduced pressure to provide 2-chloro-N-cyclopropyl-5-(difluoromethyl)pyrimidin-4-amine (Intermediate 173-b) as a yellow solid (1.8 g, 50.1% yield). LCMS (ESI) m/z 220.1 [M+H]⁺.

### Step 3: 1-[4-[[4-(cyclopropylamino)-5-(difluoromethyl)pyrimidin-2-yl]amino]indazol-1-yl]-2-methyl-propan-2-ol

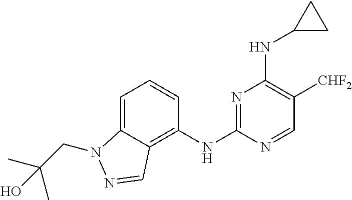 [SMILES: CC(C)(O)Cn1ncc2c(Nc3ncc(C(F)F)c(NC4CC4)n3)cccc21]

A mixture of 1-(4-aminoindazol-1-yl)-2-methyl-propan-2-ol (Intermediate 97-b) (50 mg, 0.243 mmol), 2-chloro-N-cyclopropyl-5-(difluoromethyl)pyrimidin-4-amine (Intermediate 173-b) (53.5 mg 0.243 mmol) and aq. HCl (1 M, 24.3 μL) in i-PrOH (2 mL) was degassed and purged with N₂ for 3 times, then stirred at 80° C. for 1 hr under N2 atmosphere. The reaction solution was concentrated to give a crude product. The residue was purified by Pre-HPLC (column: Boston Green ODS 150×30 mm×5 μm; mobile phase: [water (HCl)-ACN]; B %: 15%-55%, 9 min) to give the title compound as a white solid (14.1 mg, 14.3% yield). ¹H NMR (400 MHz, DMSO-d₆) δ ppm 0.57-0.96 (m, 4H), 1.12 (s, 6H), 2.93 (br d, J=4.2 Hz, 1H), 4.31 (s, 2H), 6.87-7.21 (m, 1H), 7.26-7.59 (m, 2H), 7.92 (br s, 1H), 821-8.42 (m, 2H), 8.49 (br s, 1H), 10.69 (br s, 1H); LCMS (ESI) m/z 389.3 [M+H]⁺.

### Example 174

### N4-cyclopropyl-N2-(7-methyl-1-(2-(methylsulfonyl)ethyl)-1H-indazol-4-yl)-5-(trifluoromethyl)pyrimidine-2,4-diamine

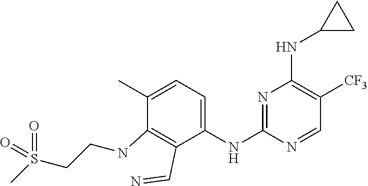 [SMILES: Cc1ccc(Nc2ncc(C(F)(F)F)c(NC3CC3)n2)c(C=N)c1NCCS(C)(=O)=O]

### Step 1: 4-bromo-7-methyl-1-(2-methylsulfonylethyl)indazole

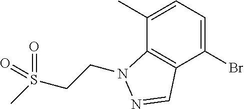 [SMILES: Cc1ccc(Br)c2cnn(CCS(C)(=O)=O)c12]

To a solution of 4-bromo-7-methyl-1H-indazole (900 mg 4.26 mmol) in DMF (10 mL) was added 1-bromo-2-methylsulfonyl-ethane (877 mg, 4.69 mmol) and Cs₂CO₃ (2.08 g, 6.40 mmol). The mixture was stirred at 80° C. for 16 hrs under N₂ atmosphere. The reaction mixture was filtered, and the filtrate was concentrated under reduced pressure to give a crude product. The crude was purified by prep-HPLC (column: C18-1 150×30 mm×5 in; mobile phase: [water (NH₃H₂O+NH₄HCO₃)-ACN]; B %: 33%-53%, 9 min) to give 4-bromo-7-methyl-1-(2-methylsulfonylethyl)indazole (Intermediate 174-a) as a white solid (450 mg, 33.3% yield). ¹H NMR (400 MHz, DMSO-d₆) δ ppm 2.70-2.79 (m, 3H), 2.97 (s, 3H), 3.68-3.85 (m, 2H), 5.01 (t, J=7.15 Hz, 2H), 7.07-7.13 (m, 1H), 7.23-7.32 (m, 1H), 8.03-8.15 (m, 1H); LCMS (ESI) m/z 317.1 [M+H]⁺.

### Step 2: tert-butyl N-[7-methyl-1-(2-methylsulfonylethyl)indazol-4-yl]carbamate

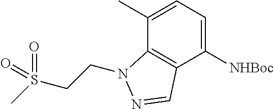 [SMILES: Cc1ccc(NC(=O)OC(C)(C)C)c2cnn(CCS(C)(=O)=O)c12]

To a solution of 4-bromo-7-methyl-1-(2-methylsulfonylethyl)indazole (Intermediate 174-a) (400 mg, 1.26 mmol), tert-butyl carbamate (177 mg, 1.51 mmol), XPhos Pd G₃ (107 mg, 0.126 mmol) and K₃PO₄ (268 mg, 1.26 mmol) in dioxane (5 mL) were stirred at 100° C. for 16 hrs under N₂ atmosphere. The reaction mixture was filtered and the filtrate was concentrated under reduced pressure to give a crude product. The crude was purified by flash silica gel chromatography (ISCO®; 12 g SepaFlash® Silica Flash Column, Eluent of 0˜50% Ethyl acetate/Petroleum ether gradient @ 12 mL/min, 254 nm) to give tert-butyl N-[7-methyl-1-(2-methylsulfonylethyl)indazol-4-yl]carbamate (Intermediate 174-b) as a white solid (120 mg, 26.9% yield). ¹H NMR (400 MHz, DMSO-d₆) δ ppm 1.51 (s, 9H), 2.47 (s, 3H), 2.93 (s, 3H), 3.68-3.79 (m, 2H), 3.88-3.92 (m, 2H), 7.02-7.08 (m, 1H), 7.31-7.44 (m, 1H), 8.41 (s, 1H); LCMS (ESI) m/z 354.1 [M+H]⁺.

### Step 3: 7-methyl-1-(2-methylsulfonylethyl)indazol-4-amine

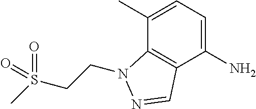 [SMILES: Cc1ccc(N)c2cnn(CCS(C)(=O)=O)c12]

To a solution of tert-butyl N-[7-methyl-1-(2-methylsulfonylethyl)indazol-4-yl]carbamate (Intermediate 174-b) (110 mg, 0.311 mmol) was added in 2M HCl in MeOH (2 mL). The mixture was stirred at 25° C. for 1 hr under N₂ atmosphere. The residue was quenched by aq. NaHCO₃ (2 mL) and extracted with EtOAc. The organic layer was then concentrated to afford the crude product 7-methyl-1-(2-methylsulfonylethyl)indazol-4-amine (Intermediate 174-c) as a yellow solid (50 mg, 63.4% yield), which was used into the next step without further purification. LCMS (ESI) m/Z 254.1 [M+H]⁺.

### Step 4: N⁴-cyclopropyl-N²-(7-methyl-1-(2-(methylsulfonyl)ethyl)-1H-indazol-4-yl)-5-(trifluoromethyl)pyrimidine-2,4-diamine

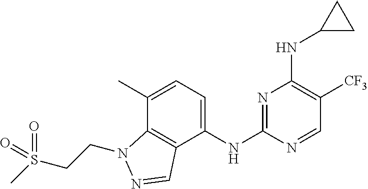 [SMILES: Cc1ccc(Nc2ncc(C(F)(F)F)c(NC3CC3)n2)c2cnn(CCS(C)(=O)=O)c12]

A mixture of 7-methyl-1-(2-methylsulfonylethyl)indazol-4-amine (Intermediate 174-c) (50 mg, 0.197 mmol), 2-chloro-N-cyclopropyl-5-(trifluoromethyl)pyrimidin-4-amine (Intermediate 68-a) (46.9 mg, 0.197 mmol) and aq. HCl (1 M, a drop) in i-PrOH (1 mL) was stirred at 80° C. for 1 hr under N₂ atmosphere. The reaction mixture was concentrated under reduced pressure to give a crude product. The crude was purified by prep-HPLC (column: C18-1 150×30 mm×5 □m; mobile phase: [water (NH₃H₂O+NH₄HCO₃)-ACN]; B %: 36%-76%, 9 min) to give the title compound as a white solid (15 mg, 16.7% yield), ¹H NMR (400 MHz, DMSO-d₆) δ ppm 0.68-0.72 (m, 2H), 0.78-0.79 (m, 2H), 2.69 (br s, 3H), 2.86 (br s, 1H), 2.94 (br s, 3H), 3.74 (br s, 2H), 4.97 (br s, 2H), 7.04-7.25 (m, 2H), 8.01-8.15 (m, 1H), 8.23 (s, 1H), 8.54 (s, 1H), 9.75 (s, 1H); LCMS (ESI) m/z 455.1 [M+H]⁺.

### Example 175

### 1-[4-[[4-(cyclopropylamino)-5-(difluoromethyl)pyrimidin-2-yl]amino]-7-fluoro-indazol-1-yl]-2-methyl-propan-2-ol

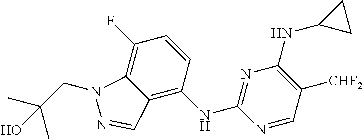 [SMILES: CC(C)(O)Cn1ncc2c(Nc3ncc(C(F)F)c(NC4CC4)n3)ccc(F)c21]

A mixture of 1-(4-amino-7-fluoro-indazol-1-yl)-2-methyl-propan-2-ol (Intermediate 172-c) (50 mg, 0.223 mmol), 2-chloro-N-cyclopropyl-5-(difluoromethyl) pyrimidin-4-amine (Intermediate 173-b) (49.1 mg, 0.223 mmol) and aq. HCl (1 M, 224 μL) in i-PrOH (2 mL) was stirred at 80° C. for 1 hr. i-PrOH was evaporated, and the residue was purified by prep-HPLC (column: Boston Green ODS 150×30 mm×5 μm; mobile phase: [water (HCl)-ACN]; B %: 5%-45%, 9 min) to give the title compound as a white solid (19.9 mg, 21.8% yield). ¹H NMR (400 MHz, DMSO-d₆) δ ppm 0.67-0.89 (m, 4H), 1.10 (s, 6H), 2.79-2.95 (m, 1H), 4.39 (s, 2H), 6.84-7.12 (m, 1H), 7.19-7.31 (m, 1H) 7.81 (br d. J=5.50 Hz, 1H), 8.33 (br s, 1H), 8.53 (s, 1H), 8.83 (s, 1H), 11.16 (s, 1H); LCMS (ESI) m/z 407.0 [M+H]⁺.

### Example 176

### 1-[4-[[5-chloro-4-(ethylamino)-7H-pyrrolo[2,3-d]pyrimidin-2-yl]amino]indazol-1-yl]-2-methyl-propan-2-ol

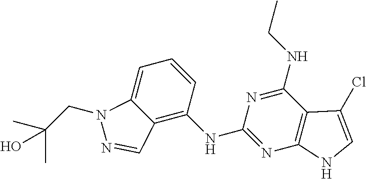 [SMILES: CCNc1nc(Nc2cccc3c2cnn3CC(C)(C)O)nc2[nH]cc(Cl)c12]

### Step 1: 2,5-dichloro-N-ethyl-7-(2-trimethylsilylethoxymethyl)pyrrolo[2,3-d]pyrimidin-4-amine

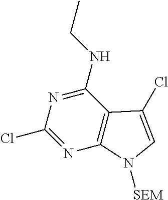 [SMILES: CCNc1nc(Cl)nc2c1c(Cl)cn2COCC[Si](C)(C)C]

To a mixture of 2,5-dichloro-N-ethyl-7H-pyrrolo[2,3-d]pyrimidin-4-amine (Intermediate 147-b) (200 mg, 0.865 mmol) and Cs₂CO₃ (845 mg, 2.60 mmol) in THF (5 mL) was added SEM-Cl (158 mg, 0.952 mmol). The mixture was stirred under N₂ at 25° C. for 16 hrs and filtered, the filtrate was concentrated to give crude product. The residue was purified by flash chromatography (ISCO®; 4 g SepaFlash® Silica Flash Column, petroleum ether/EtOAc with EtOAc from 0˜50%, 12 mL/min, 254 nm) to give 2,5-dichloro-N-ethyl-7-(2-trimethylsilylethoxymethyl)pyrrolo[2,3-d]pyrimidin-4-amine (Intermediate 176-a) as a white solid (200 mg, 64.0% yield). ¹H NMR (400 MHz, DMSO-d₆) δ ppm −0.12-0.04 (m, 9H), 0.85-0.95 (m, 2H), 1.25 (t, J=7.1 Hz, 3H), 3.56 (br d, J/=0.8 Hz, 4H), 5.47 (s, 2H), 7.24 (t, J=5.8 Hz, 1H), 7.57 (s, 1H).

### Step 2: 1-[4-[[5-chloro-4-(ethylamino)-7-(2-trimethylsilylethoxymethyl)pyrrolo [2,3-d]pyrimidin-2-yl]amino]indazol-1-yl]-2-methyl-propan-2-ol

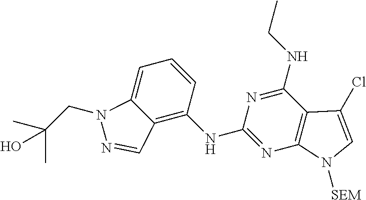 [SMILES: CCNc1nc(Nc2cccc3c2cnn3CC(C)(C)O)nc2c1c(Cl)cn2COCC[Si](C)(C)C]

A mixture of 1-(4-aminoindazol-1-yl)-2-methyl-propan-2-ol (Intermediate 97-b) (110 mg, 0.539 mmol), 2,5-dichloro-N-ethyl-7-(2-trimethylsilylethoxymethyl)pyrrolo[2,3-d]pyrimidin-4-amine (Intermediate 176-a) (150 mg, 0.415 mmol), Pd₂(dba)₃ (38.0 mg, 41.5 μmol), Xantphos (48.0 mg, 83.0 μmol) and Cs₂CO₃ (338 mg, 1.04 mmol) in toluene (5 mL) was degassed and purged with N₂ for 3 times, and then the mixture was stirred at 100° C. for 16 hrs under N₂ atmosphere. The reaction mixture was filtered and the filtrate was concentrated to give a residue. The residue was purified by flash chromatography (iSCO®; 4 g SepaFlash® Silica Flash Column, petroleum ether/EtOAc with EtOAc from 0˜50%, 12 mL/min, 254 nm) to give 1-[4-[[5-chloro-4-(ethylamino)-7-(2-trimethylsilylethoxymethyl)pyrrolo[2,3-d]pyrimidin-2-yl]amino] indazol-1-yl]-2-methyl-propan-2-ol (Intermediate 176-b) as a yellow oil (150 mg, 68.2% yield). ¹H NMR (400 MHz, DMSO-d₆) δ ppm 0.00 (s, 9H), 0.88-1.01 (m, 2H), 1.23 (s, 6H), 1.34 (t, J=7.1 Hz, 3H), 3.57-3.77 (m, 4H), 4.36 (s, 2H), 4.68-4.88 (m, 1H), 5.51 (s, 2H), 6.66 (t, J=5.8 Hz, 1H), 7.27-7.39 (m, 3H), 8.14 (d, J=7.4 Hz, 1H), 8.58 (s, 1H), 9.14 (s, 1H).

### Step 3: 1-[4-[[5-chloro-4-(ethylamino)-7-(hydroxymethyl)pyrrolo[2,3-d]pyrimidin-2-yl]amino]indazol-1-yl]-2-methyl-propan-2-ol

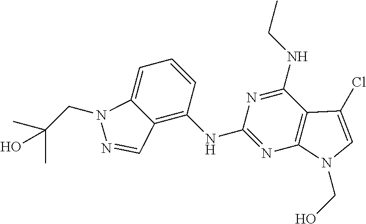 [SMILES: CCNc1nc(Nc2cccc3c2cnn3CC(C)(C)O)nc2c1c(Cl)cn2CO]

A mixture of 1-[4-[[5-chloro-4-(ethylamino)-7-(2-trimethylsilylethoxymethyl)pyrrolo[2,3-d]pyrimidin-2-yl]amino]indazol-1-yl]-2-methyl-propan-2-ol (Intermediate 176-b) (140 mg, 0.264 mmol) in TFA/DCM (3 mL, v:v=1:3) was stirred at 25° C. for 1 hr under N₂ atmosphere. The reaction solution was concentrated to give the crude product 1-[4-[[5-chloro-4-(ethylamino)-7-(hydroxymethyl)pyrrolo[2,3-d]pyrimidin-2-yl]amino]indazol-1-yl]-2-methyl-propan-2-ol (Intermediate 176-c) as a yellow oil (150 mg, 100% yield). LCMS (ESI) m/z 430.2 [M+H]⁺.

### Step 4: 1-[4-[[5-chloro-4-(ethylamino)-711-pyrrolo[2,3-d]pyrimidin-2-yl]amino] indazol-1-yl]-2-methyl-propan-2-ol

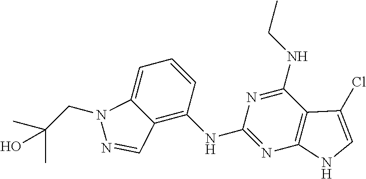 [SMILES: CCNc1nc(Nc2cccc3c2cnn3CC(C)(C)O)nc2[nH]cc(Cl)c12]

A mixture of 1-[4-[[5-chloro-4-(ethylamino)-7-(hydroxymethyl)pyrrolo[2,3-d]pyrimidin-2-yl]amino]indazol-1-yl]-2-methyl-propan-2-ol (Intermediate 176-c) (150 mg, 348 μmol) and LiOH·H₂O (83.5 mg, 3.49 mmol) in THF (2 mL) and H₂O (0.4 mL) was degassed and purged with N₂ for 3 times, and then the mixture was stirred at 25° C. for 2 hrs under N₂ atmosphere. The reaction mixture was filtered and the filtrate was concentrated to give a crude product, which was purified by flash chromatography (ISCO®; 4 g SepaFlash® Silica Flash Column, petroleum ether/EtOAc with EtOAc from 0˜50%, 12 mL/min, 254 nm) to give the title compound as a gray solid (157 mg, 95.7% yield). ¹H NMR (400 MHz, DMSO-d₆) δ ppm 1.13 (s, 6H), 1.23 (t, J=7.0 Hz, 3H), 3.52-3.67 (m, 2H), 4.25 (s, 2H), 4.67 (s, 1H), 6.45 (t, J=5.7 Hz, 1H), 6.99 (d, J=2.4 Hz, 1H), 7.15-7.31 (m, 2H), 8.02 (d, J=7.3 Hz, 1H), 8.41 (s, 1H), 8.88 (s, 1H) 11.35 (s, 1H); LCMS (ESI) m/z 400.1 [M+H]⁺.

### Example 177

### N⁴-cyclopropyl-N²-[7-fluoro-1-(2-methylsulfonylethyl)indazol-4-yl]-5-(trifluoromethyl)pyrimidine-2,4-diamine

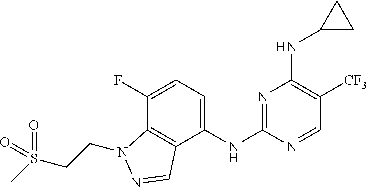 [SMILES: CS(=O)(=O)CCn1ncc2c(Nc3ncc(C(F)(F)F)c(NC4CC4)n3)ccc(F)c21]

### Step 1: 4-bromo-7-fluoro-1-(2-methylsulfonylethyl)indazole

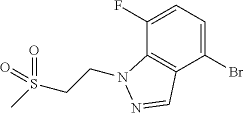 [SMILES: CS(=O)(=O)CCn1ncc2c(Br)ccc(F)c21]

A mixture of 4-bromo-7-fluoro-1H-indazole (1.5 g, 6.98 mmol), 1-bromo-2-methylsulfonyl-ethane (1.44 g, 7.67 mmol), Cs₂CO₃ (3.18 g, 9.77 mmol) in DMF (3 mL) was degassed and purged with N₂ for 3 times, and then stirred at 80° C. for 16 hrs under N₂ atmosphere. The reaction mixture was filtered and the filtrate was concentrated under reduced pressure to give a residue. The residue was purified by flash chromatography (ISCO®; 4 g SepaFlash® Silica Flash Column, petroleum ether/EtOAc with EtOAc from 0˜50%, 12 mL/min, 254 nm) to provide 4-bromo-7-fluoro-1-(2-methylsulfonylethyl)indazole (Intermediate 177-a) as a white solid (1.14 g, 50.9% yield). ¹H NMR (400 MHz, DMSO-d₆) δ ppm 2.96 (s, 3H), 3.78 (t, J=6.71 Hz, 2H), 4.93 (t, J=6.82 Hz, 2H), 7.26 (br d, J=11.88 Hz, 1H), 7.34 (br d, J=3.52 Hz, 1H), 8.18 (d, J=1.98 Hz, 1H).

### Step 2: tert-butyl N-[7-fluoro-1-(2-methylsulfonylethyl)indazol-4-yl]carbamate

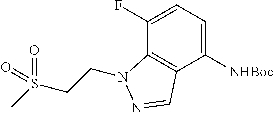 [SMILES: CC(C)(C)OC(=O)Nc1ccc(F)c2c1cnn2CCS(C)(=O)=O]

A mixture of 4-bromo-7-fluoro-1-(2-methylsulfonylethyl) indazole (Intermediate 177-a) (1.12 g, 3.49 mmol), tert-butyl carbamate (490 mg, 4.18 nmol), K₃PO₄ (2.22 g, 10.5 mmol), XPhos Pd G3 (295 mg, 0.348 mmol) in dioxane (10 mL) was degassed and purged with N₂ for 3 times, and then stirred at 90° C. for 12 hrs under N₂ atmosphere. The reaction mixture was filtered and the filtrate was concentrated under reduced pressure to give a residue, which was purified by flash silica gel chromatography (ISCO®; 20 g SepaFlash® Silica Flash Column, Eluent of 0˜100% ethyl acetate/Petroleum ether gradient @0.50 mL/min, 254 nm) to give tert-butyl N-[7-fluoro-1-(2-methylsulfonylethyl)indazol-4-yl]carbamate (Intermediate 177-b) as a white solid (220 mg, 17.6% yield). ¹H NMR (400 MHz, DMSO-d₆) δ ppm 1.45-1.61 (m, 9H), 2.94 (s, 3H), 3.74 (t, J=6.82 Hz, 2H), 4.87 (t, J=6.93 Hz, 2H), 7.02-7.25 (m, 1H), 7.30-7.53 (m, 1H), 8.32-8.56 (m, 1H), 9.50-9.71 (m, 1H).

### Step 3: 7-fluoro-1-(2-methylsulfonylethyl)indazol-4-amine

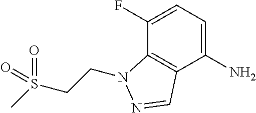 [SMILES: CS(=O)(=O)CCn1ncc2c(N)ccc(F)c21]

A mixture of tert-butyl N-[7-fluoro-1-(2-methylsulfonylethyl)indazol-4-yl]carbamate (Intermediate 177-b) (210 mg, 0.587 mmol) in 4N HCl in MeOH (2 mL) was degassed and purged with N₂ for 3 times, and then stirred at 25° C. for 1 hr under N₂ atmosphere. The reaction mixture was filtered and the filtrate was concentrated under reduced pressure to give 7-fluoro-1-(2-methylsulfonylethyl)indazol-4-amine (Intermediate 177-c) as a white solid (120 mg, 79.4% yield). LCMS (ESI) m/z 258.1 [M+H]⁺.

### Step 4: N⁴-cyclopropyl-N²-[7-fluoro-1-(2-methylsulfonylethyl)indazol-4-yl]-5-(trifluoromethyl)pyrimidine-2,4-diamine

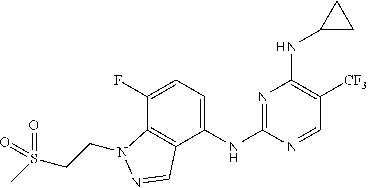 [SMILES: CS(=O)(=O)CCn1ncc2c(Nc3ncc(C(F)(F)F)c(NC4CC4)n3)ccc(F)c21]

A mixture of 7-fluoro-1-(n-methylsulfonylethyl)indazol-4-amine (Intermediate 177-c) (120 mg, 0.466 mmol), 2-chloro-N-cyclopropyl-5-(trifluoromethyl)pyrimidin-4-amine (Intermediate 68-a) (110 mg, 0.466 mmol) and aq. HC (1 M, 466 μL) in i-PrOH (1 mL) was degassed and purged with N₂ for 3 times, and then stirred at 80° C. for 1 hr under N₂ atmosphere. The reaction mixture was filtered and concentrated under reduced pressure to give a residue, which was purified by prep-HPLC (column: Boston Green ODS 150×30 mm×5 μm; mobile phase: [water(HCl)-ACN]; B %: 0%-60%, 9 min) to give the title compound as a white solid (86 mg, 40.2% yield). ¹H NMR (400 MHz, DMSO-d₆) δ ppm 0.65-0.72 (m, 2H), 0.75-0.82 (m, 2H), 2.79-2.89 (m, 1H), 2.91 (s, 3H), 3.74-3.80 (m, 2H), 4.82-5.03 (m, 2H), 7.13-7.33 (m, 1H), 7.43-7.64 (m, 1H), 7.97-8.13 (m, 1H), 8.27 (s, 1H), 8.51 (s, 1H), 10.11 (s, 1H); LCMS (ESI) m/z 459.1 [M+H]⁺.

### Example 178

### N⁴-methyl-N²-(9-oxa-1,2-diazatricyclo[6.3.1.04,12]dodeca-2,4,6,8(12)-tetraen-5-yl)-5-(trifluoromethyl)pyrimidine-2,4-diamine

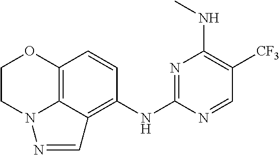 [SMILES: CNc1nc(Nc2ccc3c4c2cnn4CCO3)ncc1C(F)(F)F]

### Step 1: 4-bromo-1H-indazol-7-ol

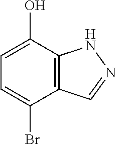 [SMILES: Oc1ccc(Br)c2cn[nH]c12]

To a solution of 4-bromo-7-methoxy-1H-indazole (2 g, 8.81 mmol) in DCM (40 mL) was added BBr₃ (8.50 mL, 88.0 mmol) at 0° C., then the mixture was stirred at 25° C. for 16 hrs under N₂ atmosphere. The solution was quenched by aq. NaHCO₃ (100 mL), extracted with EtOAc (100 mL×2), washed with brine (100 mL×2), dried over Na₂SO₄ to give 4-bromo-1H-indazol-7-ol (Intermediate 178-a) as a white solid (1.4 g, 74.6% yield), ¹H NMR (400 MHz, DMSO-d₆) δ ppm 6.52-6.75 (m, 1H), 7.08 (d, J=7.9 Hz, 1H), 7.91 (d, J=0.8 Hz, 1H), 10.43 (s, 1H), 13.46 (br s, 1H).

### Step 2: 5-bromo-9-oxa-1,2-diazatricyclo[6.3.1.04,12]dodeca-2,4,6,8(12)-tetraene

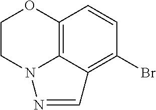 [SMILES: Brc1ccc2c3c1cnn3CCO2]

A mixture of 4-bromo-1H-indazol-7-ol (Intermediate 178-a) (1 g, 4.69 mmol), 1,2-dibromoethane (970 mg, 5.16 mmol) and Cs₂CO₃ (4.59 g, 14.0 mmol) in DMF (20 mL) was stirred at 80° C. for 1 hr under N₂ atmosphere. The mixture was filtered and the filtrate was concentrated. The residue was purified by flash chromatography (ISCO®; 12 g SepaFlash® Silica Flash Column, petroleum ether/EtOAc with EtOAc from 0˜50%, 18 mL/min, 254 mn) to give 5-bromo-9-oxa-1,2-diazatricyclo [6.3.1.04,12]dodeca-2,4,6,8(12)-tetraene (Intermediate 178-b) as a white solid (400 mg, 356% yield). ¹H NMR (400 MHz, DMSO-d₆) δ ppm 4.51-4.59 (m, 2H), 4.62-4.72 (m, 2H), 6.72 (d, J=7.9 Hz, 1), 7.20 (d, J=7.9 Hz, 1H), 7.89-8.06 (m, 1H).

### Step 3: N-(9-oxa-1,2-diazatricyclo[6.3.1.04,12]dodeca-2,4,6,8(12)-tetraen-5-yl)-1,1-diphenyl-methanimine

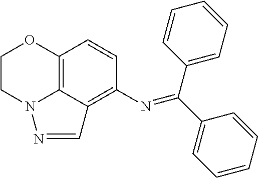 [SMILES: c1ccc(C(=Nc2ccc3c4c2cnn4CCO3)c2ccccc2)cc1]

A mixture of 5-bromo-9-oxa-1,2-diazatricyclo [6.3.1.04,12]dodeca-2,4,6,8(12)-tetraene (Intermediate 178-b) (400 mg, 1.67 mmol), diphenylmethanimine (333 mg, 1.84 mmol), Cs₂CO₃ (1.64 g, 5.02 mmol) in dioxane (8 mL) was added XantPhos Pd G₃ (158 mg, 0.167 nmol), and then the mixture was stirred at 100° C. for 16 hrs under N₂ atmosphere, then concentrated to give a crude product, which was purified by flash chromatography (ISCO®; 12 g SepaFlash® Silica Flash Column, petroleum ether/EtOAc with EtOAc from 0˜50%, 12 mL/min, 254 nm) to give N-(9-oxa-1,2-diazatricyclo[6.3.1.04,12]dodeca-2,4,6,8(12)-tetraen-5-yl)-1,1-diphenyl-methanimine (Intermediate 178-c) as a white solid (300 ng, 52.8% yield). ¹H NMR (400 MHz, DMSO-d₆) δ ppm 4.38-4.46 (m, 2H), 4.48-4.56 (m, 2H), 6.18 (d, J=7.7 Hz, 1H), 6.53 (d, J=7.7 Hz, 1H), 7.15-7.23 (m, 2), 7.25-7.32 (m, 3H), 7.46-7.52 (m, 2H), 7.53-7.58 (m, 1H), 7.67-7.73 (m, 2H), 7.80 (s, 1H); LCMS (ESI) m/z 340.1 [M+H]⁺.

### Step 4: 9-oxa-1,2-diazatricyclo[6.3.1.04,12]dodeca-2,4,6,8(12)-tetraen-5-amine

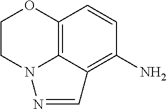 [SMILES: Nc1ccc2c3c1cnn3CCO2]

A mixture of N-(9-oxa-1,2-diazatricyclo[6.3.1.04,12]dodeca-2,4,6,8(12)-tetraen-5-yl)-1,1-diphenyl-methanimine (Intermediate 178-c) (300 mg, 0.883 mmol), NaOAc (181 mg, 2.21 mmol) and NH₂OH—HCl (122 mg, 1.77 mmol) in MeOH (15 mL) was stirred at 25° C. for 1 hr under N₂ atmosphere, then concentrated to give a crude product, which was purified by flash chromatography (ISCO®; 4 g SepaFlash® Silica Flash Column, petroleum ether/EtOAc with EtOAc from 0˜50%, 12 mL/min, 254 mn) to give 9-oxa-1,2-diazatricyclo[6.3.1.04,12]dodeca-2,4,6,8(12)-tetraen-5-amine (Intermediate 178-d) as a white solid (120 mg, 77.5% yield). ¹H NMR (400 MHz, DMSO-d₆) δ ppm 4.22-4.49 (m, 4H), 5.32 (s, 2H), 5.96 (d, J=7.7 Hz, 1H), 6.44 (d, J=7.7 Hz, 1H), 7.98 (s, 1H); LCMS (ESI) m/z 176.1 [M+H]⁺.

### Step 5: N⁴-methyl-N²-(9-oxa-1,2-diazatricyclo[6.3.1.04,12]dodeca-2,4,6,8(12)-tetraen-5-yl)-5-(trifluoromethyl)pyrimidine-2,4-diamine

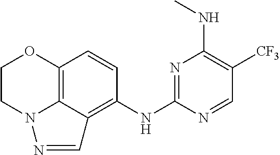 [SMILES: CNc1nc(Nc2ccc3c4c2cnn4CCO3)ncc1C(F)(F)F]

A mixture of 9-oxa-1,2-diazatricyclo[6.3.1.04,12]dodeca-2,4,6,8(12)-tetraen-5-amine (Intermediate 178-d) (30 mg, 0.171 mmol), 2-chloro-N-methyl-5-(trifluoromethyl)pyrimidin-4-amine (Intermediate 1-a) (36.2 mg, 0.171 mmol) and aq. HC (1 M, 17.1 μL) in i-PrOH (3 mL) was stirred at 80° C. for 1 hr under N₂ atmosphere. The reaction mixture was filtered and the filter cake was triturated with EtOAc (10 mL×2) to give the title compound as a white solid (29.8 mg, 49.6% yield). ¹H NMR (400 MHz, DMSO-d₆) δ ppm 2.88 (br d, J=4.2 Hz, 3H), 4.45-4.71 (m, 4H), 6.77 (d, J=8.1 Hz, 1H), 7.35 (br d, J=7.9 Hz, 1H), 8.20 (s, 2H), 8.36 (br s, 1H), 10.81 (br s, 1H); LCMS (ESI) m/z 351.3 [M+H]⁺.

### Example 179

### N⁴-cyclopropyl-N²-(2,3-dihydro-[1,4]oxazino[2,3,4-hi]indazol-7-yl)-5-(trifluoromethyl)pyrimidine-2,4-diamine

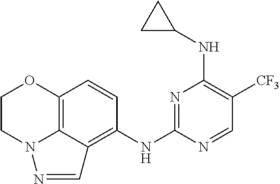 [SMILES: FC(F)(F)c1cnc(Nc2ccc3c4c2cnn4CCO3)nc1NC1CC1]

A mixture of 9-oxa-1,2-diazatricyclo[6.3.1.0^{4,12}]dodeca-2,4,6,8(12)-tetraen-5-amine (Intermediate 178-d) (20 mg, 0.114 mmol), 2-chloro-N-cyclopropyl-5-(trifluoromethyl)pyrimidin-4-amine (Intermediate 68-a) (27.1 mg, 0.114 mmol) and aq. HCl (1 M, 0.114 mmol) in i-PrOH (1 mL) was stirred at 80° C. for 1 hr under N₂ atmosphere. The precipitate was formed, collected by filtration and triturated with EtOAc (10 mL) to provide the title compound as a white solid (20 mg, 46.6% yield). ¹H NMR (400 MHz, DMSO-d₆) δ ppm 0.71-0.78 (m, 4H), 2.84-2.97 (m, 1H), 4.50-4.54 (m, 2H), 4.58-4.63 (m, 2H), 6.49-6.87 (m, 1H), 7.71 (br s, 1H), 8.12 (br s, 1H), 8.30 (br s, 1H), 8.42 (br s, 1H), 10.78 (br s, 1H); LCMS (ESI) m/z 377.1 [M+H]⁺.

### Example 18

### N⁴-isopropyl-N²-(9-oxa-1,2-diazatricyclo[6.3.1.04,12]dodeca-2,4,6,8(12)-tetraen-5-yl)-5-(trifluoromethyl)pyrimidine-2,4-diamine

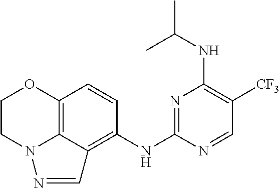 [SMILES: CC(C)Nc1nc(Nc2ccc3c4c2cnn4CCO3)ncc1C(F)(F)F]

A mixture of 9-oxa-1,2-diazatricyclo[6.3.1.04,12]dodeca-2,4,6,8(12)-tetraen-5-amine (Intermediate 178-d) (40 ng, 0.228 mmol) 2-chloro-N-isopropyl-5-(trifluoromethyl)pyrimidin-4-amine (Intermediate 140-a) (54.7 mg, 0.228 mmol) and aq. HCl (1 M, 228 L) in i-PrOH (3 mL) was degassed and purged with N₂ for 3 times, and then stirred at 80° C. for 1 hr under N₂ atmosphere. The reaction solution was concentrated to give a crude product, which was triturated with EtOAc (10 rnL); to give the title compound as a gray solid (73 mg, 77.1% yield). ¹H NMR (400 MHz, DMSO-d₆) δ ppm 1.18 (d, J=6.6 Hz, 6H), 4.40 (br s, 1H), 4.49-4.56 (m, 2H), 4.59-4.68 (m, 2H), 6.75 (d, J=7.9 Hz, 1H), 7.30 (br d, J=7.7 Hz, 1H), 7.47 (br s, 1H), 8.16 (s, 1H), 8.28 (br s, 1H), 10.45 (br s, 1H); LCMS (ESI) m/z 379.0 [M+H]⁺.

### Example 181

### 1-(4-((3-chloro-4-(ethylamino)-1H-pyrazolo[3,4-d]pyrimidin-6-yl)amino)-7-fluoro-1H-indazol-1-yl)-2-methylpropan-2-ol

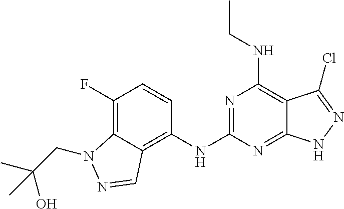 [SMILES: CCNc1nc(Nc2ccc(F)c3c2cnn3CC(C)(C)O)nc2[nH]nc(Cl)c12]

### Step 1: 6-chloro-N-ethyl-1H-pyrazolo[3,4-d]pyrimidin-4-amine

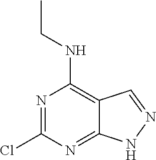

To a solution of 4,6-dichloro-2H-pyrazolo[3,4-d]pyrimidine (2 g, 10.6 mmol) in MeOH (20 mL) was added ethanamine hydrochloride (863 mg, 10.6 mmol) and TEA (3.21 g, 31.8 mmol). The mixture was stirred at 80° C. for 2 hrs under N₂ atmosphere. The reaction mixture was filtered and the filtrate was concentrated under reduced pressure to give a crude product. The crude was purified by flash silica gel chromatography (ISCO®; 40 g SepaFlash® Silica Flash Column, Eluent of 0-50% Ethyl acetate/Petroleum ethergradient @ 25 mL/min, 254 nm) to give 6-chloro-N-ethyl-1H-pyrazolo[3,4-d]pyrimidin-4-amine (Intermediate 181-a) as a yellow solid (1.5 g, 71.77% yield). ¹H NMR (400 MHz, DMSO-d₆) δ (ppm 1.18-1.23 (m, 3H), 3.42-3.53 (m, 2H), 7.99-8.24 (m, 1H), 8.50-8.71 (m, 1H), 13.32-13.67 (m, 1H); LCMS (ESI) m/z 198.1 [M+H]⁺.

### Step 2: tert-butyl N-[7-methyl-1-(2-methylsulfonylethyl)indazol-4-yl]carbamate

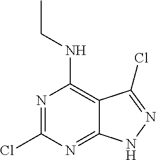

A mixture of 6-chloro-N-ethyl-1H-pyrazolo[3,4-d]pyrimidin-4-amine (Intermediate 181-a) (1.5 g, 7.59 mmol) and NCS (2.03 g, 15.2 mmol) in DMF (15 mL) was stirred at 80° C. for 3 hrs under N₂ atmosphere. The resulting mixture was filtered and the filtrate was concentrated under reduced pressure to give a crude product, which was purified by flash silica gel chromatography (ISCO®; 12 g SepaFlash® Silica Flash Column, Eluent of 0˜50% Ethyl acetate/Petroleum ether gradient 25 mL/min, 254 nm) to give 3,6-dichloro-N-ethyl-1H-pyrazolo[3,4-d]pyrimidin-4-amine (Intermediate 181-b) as a white solid (1 g, 56.8% yield). ¹H NMR (400 MHz, DMSO-d₆) δ ppm 1.15-1.20 (m, 3H), 3.49-3.58 (m, 2H), 7.76 (br s, 1H), 13.81 (s, 1H); LCMS (ESI) m/z 232.1 [M+H]⁺.

### Step 3: 3,6-dichloro-N-ethyl-1-(2-trimethylsilylethoxymethyl)pyrazolo[3,4-d]pyrimidin-4-amine

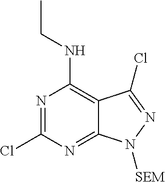 [SMILES: CCNc1nc(Cl)nc2c1c(Cl)nn2COCC[Si](C)(C)C]

To a mixture of 3,6-dichloro-N-ethyl-1H-pyrazolo[3,4-d]pyrimidin-4-amine (Intermediate 181-b) (1 g, 4.31 mmol) and Cs₂CO₃ (4.21 g, 12.9 mmol) in DMF (10 mL) was added SEM-Cl (1.58 g, 9.48 mmol). The resulting mixture was stirred at 50′C for 16 hrs under N₂ atmosphere and then filtered, the filtrate was concentrated under reduced pressure to give a crude product, which was purified by flash silica gel chromatography (ISCO®; 40 g SepaFlash® Silica Flash Column, Eluent of 0˜40% Ethyl acetate/Petroleum ether gradient @ 25 mL/min, 254 nm) to give 3,6-dichloro-N-ethyl-1-(2-trimethylsilylethoxymethyl)pyrazolo[3,4-d]pyrimidin-4-amine (Intermediate 181-c) as a white oil (400 mg, 25.6% yield). ¹H NMR (400 MHz, DMSO-d₆) δ ppm −0.01-0.01 (m, 9H), 0.91 (t, J=8.00 Hz, 2H), 1.25-1.28 (m, 3H), 3.60 (br d, J=6.50 Hz, 2H), 3.63-3.67 (m, 2H), 5.57 (s, 2H), 7.96-8.01 (m, 1H); LCMS (ESI) m/z 362.1, 364.1 [M+H]⁺.

### Step 4: 1-[4-[[3-chloro-4-(ethylamino)-1-(2-trimethylsilylethoxymethyl) pyrazolo[3,4-d]pyrimidin-6-yl]amino]-7-fluoro-indazol-1-yl]-2-methyl-propan-2-ol

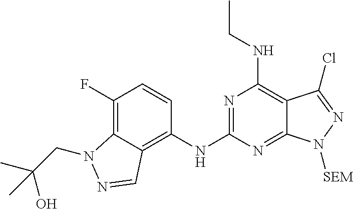 [SMILES: CCNc1nc(Nc2ccc(F)c3c2cnn3CC(C)(C)O)nc2c1c(Cl)nn2COCC[Si](C)(C)C]

A mixture of 3,6-dichloro-N-ethyl-1-(2-trimethylsilylethoxymethyl)pyrazolo[3,4-d]pyrimidin-4-amine (Intermediate 181-c) (400 mg, 1.10 mmol), 1-(4-amino-7-fluoro-indazol-1-yl)-2-methyl-propan-2-ol (Intermediate 172-c) (320 mg, 1.44 mmol), Pd)(dba)₃ (101 mg, 0.110 mmol), Xantphos (128 mg, 0.221 mmol) and Cs₂CO₃ (359 mg, 1.10 mmol) in toluene (5 mL) was stirred at 100° C. for 16 hrs under N₂ atmosphere. The resulting mixture was filtered and the filtrate was concentrated under reduced pressure to give a crude product. The crude product was purified by flash silica gel chromatography (ISCO®; 12 g SepaFlash® Silica Flash Column, Eluent of 0˜40% Ethyl acetate/Petroleum ether gradient @ 25 mL/min, 254 nm) to provide 1-[4-[[3-chloro-4-(ethylamino)-1-(2-trimethylsilylethoxymethyl)pyrazolo[3,4-d]pyrimidin-6-yl]amino]-7-fluoro-indazol-1-yl]-2-methyl-propan-2-ol (Intermediate 181-d) as a white solid (300 mg, 49.5% yield). ¹H NMR (400 MHz, DMSO-d₆) δ ppm 0.00 (s, 9H), 0.92-0.97 (m, 2H), 1.22 (s, 6H), 1.29-1.33 (m, 3H), 3.67 (br d, J=8.25 Hz, 4H), 4.48 (s, 2H), 4.82 (s, 1H), 5.55 (s, 2H), 7.23 (dd, J=12.01, 8.38 Hz, 1H), 7.28 (br t, J=5.88 Hz, 1H), 7.82-7.86 (m, 1H), 8.55-8.57 (m, 1H), 9.59-9.62 (m, 1H); LCMS (ESI) m/z 549.1 [M+H]⁺.

### Step 5: 1-[4-[[3-chloro-4-(ethylamino)-1-(hydroxymethyl)pyrazolo[3,4-d]pyrimidin-6-yl]amino]-7-fluoro-indazol-1-yl]-2-methyl-propan-2-ol

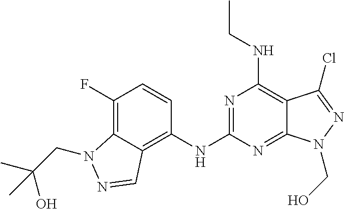 [SMILES: CCNc1nc(Nc2ccc(F)c3c2cnn3CC(C)(C)O)nc2c1c(Cl)nn2CO]

A solution of 1-[4-[[3-chloro-4-(ethylamino)-1-(2-trimethylsilylethoxymethyl)pyrazolo[3,4-d]pyrimidin-6-yl]amino]-7-fluoro-indazol-1-yl]-2-methyl-propan-2-ol (Intermediate 181-d) (100 mg, 0.182 mmol) in TFA/DCM (1 mL, v/v=1/3) was stirred at 25° C. for 2 hrs under N₂ atmosphere. The reaction mixture was filtered and the filtrate was concentrated under reduced pressure to give 1-[4-[[3-chloro-4-(ethylamino)-1-(hydroxymethyl)pyrazolo[3,4-d]pyrimidin-6-yl]amino]-7-fluoro-indazol-1-yl]-2-methyl-propan-2-ol (Intermediate 181-e) as a white solid (100 mg, 61.2% yield), which was used into the next step without further purification. LCMS (ESI) m/z 449.1 [M+H]⁺.

### Step 6: N⁴-cyclopropyl-N²-(7-methyl-1-(2-(methylsulfonyl)ethyl)-1H-indazol-4-yl)-5-(trifluoromethyl)pyrimidine-2,4-diamine

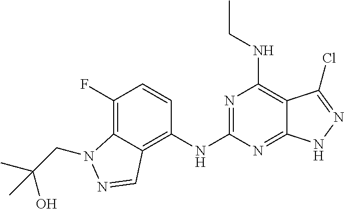 [SMILES: CCNc1nc(Nc2ccc(F)c3c2cnn3CC(C)(C)O)nc2[nH]nc(Cl)c12]

A solution of 1-[4-[[3-chloro-4-(ethylamino)-1-(hydroxymethyl)pyrazolo[3,4-d]pyrimidin-6-yl]amino]-7-fluoro-indazol-1-yl]-2-methyl-propan-2-ol (Intermediate 181-e) (100 mg, 0.223 mmol) and LiOH (26.7 mg, 1.11 mmol) in THF/H₂O (1 mL) was stirred at 25° C. for 2 hrs. The reaction mixture was filtered and the filtrate was concentrated under reduced pressure to give a crude product, which was purified by prep-HPLC (column: Boston Prime C18 150×30 mm×5 □m; mobile phase: [water (ammonia hydroxide v/v)-ACN]; 13%: 33%-53%, 11 min) to give the title compound as a white solid (17 mg, 18.2% yield). ¹H NMR (400 MHz, CD₃OD) δ (ppm 1.23 (s, 6H), 1.27-1.32 (m, 3H), 3.60-3.67 (m, 2H), 4.50 (s, 2H), 7.03-7.10 (m, 1H), 7.71-7.79 (m, 1H), 8.25 (d, J=1.88 Hz, 1H); LCMS (ESI) m/z 419.1 [M+H]⁺.

### Example 182

### N⁴-cyclopropyl-3-methyl-N⁶-[1-(2-methylsulfonylethyl)indazol-4-yl]-1H-pyrazolo[3,4-d]pyrimidine-4,6-diamine

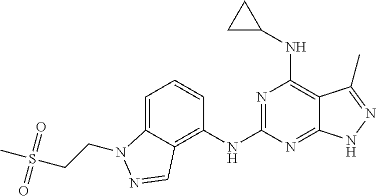 [SMILES: Cc1n[nH]c2nc(Nc3cccc4c3cnn4CCS(C)(=O)=O)nc(NC3CC3)c12]

### Step 1: 2-[(4,6-dichloro-3-methyl-pyrazolo[3,4-d]pyrimidin-1-yl)methoxy]ethyl-trimethyl-silane

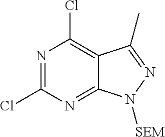 [SMILES: Cc1nn(COCC[Si](C)(C)C)c2nc(Cl)nc(Cl)c12]

To a solution of 4,6-dichloro-3-methyl-1H-pyrazolo[3,4-d]pyrimidine (1.7 g 8.37 mmol) in MeCN (1 mL) was added SEM-Cl (1.40 g, 8.37 mmol) and Cs₂)CO₃ (8.18 g, 25.1 mmol). The mixture was stirred at 25° C. for 16 hrs, diluted with H₂O 30 mL and extracted with EtOAc (90 mL). The combined organic layers were washed with brine (60 mL), dried with anhydrous Na₂SO, filtered and concentrated under reduced pressure. The residue was purified by flash silica gel chromatography (ISCO®; 4 g SepaFlash® Silica Flash Column, Eluent of 0˜7% petroleum ether/EtOAc @ 35 mL/min) to give 2-[(4,6-dichloro-3-methyl-pyrazolo[3,4-d]pyrimidin-1-yl)methoxy]ethyl-trimethyl-silane (Intermediate 182-a) as a white solid (1.3 g, 46.5% yield). LCMS (ESI) m/z 331.1 [M+H]⁺.

### Step 2: 6-chloro-N-cyclopropyl-3-methyl-1-(2-trimethylsilylethoxymethyl) pyrazolo[3,4-d]pyrimidin-4-amine

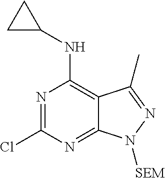 [SMILES: Cc1nn(COCC[Si](C)(C)C)c2nc(Cl)nc(NC3CC3)c12]

To a solution of 2-[(4,6-dichloro-3-methyl-pyrazolo[3,4-d]pyrimidin-1-yl)methoxy]ethyl-trimethyl-silane (Intermediate 182-a) (600 mg, 1.80 nmol) in MeCN (6 mL) was added cyclopropanamine (123 mg, 2.16 mmol) and K₂CO₃ (746 mg, 5.40 mmol). The mixture was stirred at 25° C. for 16 hrs., diluted with H₂O (30 mL) and extracted with EtOAc (90 mL). The combined organic layers were washed with brine (60 mL), dried with anhydrous Na₂SO₄, filtered and concentrated under reduced pressure to give a residue. The residue was purified by flash silica gel chromatography (ISCO®; 40 g SepaFlash® Silica Flash Column, Eluent of 0˜20% petroleum ether/EtOAc @(35 mL/min) to give 6-chloro-N-cyclopropyl-3-methyl-1-(2-trimethylsilylethoxymethyl)-pyrazolo[3,4-d]pyrimidin-4-amine (Intermediate 182-b) as a white solid (621 mg, 95.8% yield). LCMS (ESI) m/z 354.2 [M+H]⁺.

### Step 3: N⁴-cyclopropyl-3-methyl-AX-[1-(2-methylsulfonylethyl)indazol-4-yl]-1(2-trimethylsilylethoxymethyl)pyrazolo[3,4-d]pyrimidine-4,6-diamine

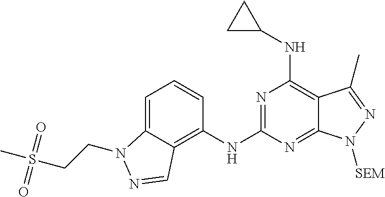 [SMILES: Cc1nn(COCC[Si](C)(C)C)c2nc(Nc3cccc4c3cnn4CCS(C)(=O)=O)nc(NC3CC3)c12]

A mixture of 1-(2-methylsulfonylethyl)indazol-4-amine (Intermediate 139-b) (87.9 mg, 0.367 mmol), 6-chloro-N-cyclopropyl-3-methyl-1-(2-trimethylsilylethoxymethyl)pyrazolo[3,4-d]pyrimidin-4-amine (Intermediate 182-b) (100 mg, 0.282 mmol), Pd₂(dba)₃ (25.8 mg, 28.2 μmol), Xantphos (32.7 mg, 56.5 μmol) and Cs₂CO₃ (230 mg, 0,706 mmol) in toluene (3 mL) was degassed and purged with N₂ for 3 times, and then stirred at 100° C. for 16 hrs under N₂ atmosphere.

The resulting mixture was concentrated under reduced pressure and the residue was purified by flash silica gel chromatography (ISCO®; 4 g SepaFlash® Silica Flash Column, Eluent of 0-100% Ethyl acetate/Petroleum ether gradient @ 12 mL/min, 254 nm) to give N⁴-cyclopropyl-3-methyl-N⁶-[1-(2-methylsulfonylethyl)indazol-4-yl]-1-(2-trimethylsilylethoxy-methyl) pyrazolo [3,4-d]pyrimidine-4,6-diamine (Intermediate 182-c) as a white solid (100 mg, 63.5% yield). LCMS (ESI) m/z 557.4 [M+H]⁺.

### Step 4: N4-cyclopropyl-3-methyl-N⁶-[1-(2-methylsulfonylethyl)indazol-4-yl]-1H-pyrazolo[3,4-d]pyrimidine-4,6-diamine

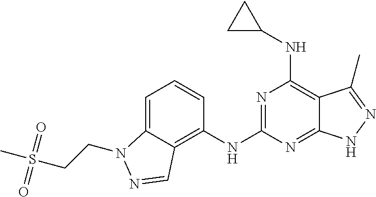 [SMILES: Cc1n[nH]c2nc(Nc3cccc4c3cnn4CCS(C)(=O)=O)nc(NC3CC3)c12]

A mixture of N⁴-cyclopropyl-3-methyl-N⁶-[1-(2-methylsulfonylethyl)indazol-4-yl]-1-(2-trimethylsilylethoxymethyl)pyrazolo[3,4-d]pyrimidine-4,6-diamine (Intermediate 182-c) (100 mg, 0.179 mmol) in TFA/DCM (5 mL; v:v=1:2) was stirred at 100° C. for 16 hrs. TFA/DCM was evaporated and the residue was purified by prep-H-PLC (column: Boston Prime C18 150×30 mm×5 μm; mobile phase: [water (HCl)-ACN]; B %: 2%-42%, 9 min) to give the title compound as a white solid (33 mg, 43.1% yield). ¹H NMR (400 MHz, DMSO-dc) δ ppm 0.73-1.10 (m, 4H), 2.55 (s, 3H), 2.90 (s, 3H), 3.03 (br s, 1H), 3.77 (t, J=6.75 Hz, 2H), 4.84 (t, J=6.75 Hz, 2H), 7.36-7.58 (m, 2H), 8.17 (br s, 1H), 8.69 (br s, 2H), 11.10 (br s, 1H), 12.49-14.32 (m, 1H); LCMS (ESI) m/z 427.1 [M+H]⁺.

### Example 183

### 1-[4-[[3-chloro-4-(methylamino)-1H-pyrazolo [3,4-d]pyrimidin-6-yl]amino]indazol-1-yl]-2-methyl-propan-2-ol

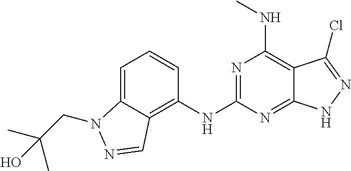 [SMILES: CNc1nc(Nc2cccc3c2cnn3CC(C)(C)O)nc2[nH]nc(Cl)c12]

### Step 1: 6-chloro-N-methyl-1H-pyrazolo[3,4-d]pyrimidin-4-amine

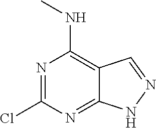

A mixture of 4,6-dichloro-1H-pyrazolo[3,4-d]pyrimidine (5 g, 26.4 mmol), methanamine (1.79 g, 26.4 mmol), TEA (8.03 g, 79.3 mmol) in MeOH (100 mL) was stirred at 80° C. for 4 hrs under N2 atmosphere. The reaction mixture was concentrated and the residue was triturated with EtOAc (100 mL) at 25° C. for 30 mins to give 6-chloro-N-methyl-1H-pyrazolo[3,4-d]pyrimidin-4-amine (Intermediate 183-a) as a yellow solid (4 g, 82.4% yield). ¹H NMR (400 MHz, DMSO-d₆) δ ppm 2.95 (d, J=4.6 Hz, 3H), 8.06 (s, 1H), 8.64 (br d, J=4.6 Hz, 1H), 13.43 (s, 1H).

### Step 2: 3,6-dichloro-N-methyl-1H-pyrazolo[3,4-d]pyrimidin-4-amine

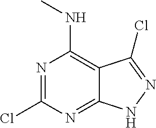

A solution of 6-chloro-N-methyl-1H-pyrazolo[3,4-d]pyrimidin-4-amine (Intermediate 183-a; (3 g, 16.3 mmol) in DMF (50 mL) and NCS (3.27 g, 24.5 mmol) was stirred at 25° C. for 16 hrs. The reaction solution was poured into H₂O (300 mL) and extracted with EtOAc (200 mL×2). The combined organic layers were washed with brine (200 mL), dried over Na₂SO₄, and concentrated tinder reduced pressure to give a yellow solid, which was purified by flash chromatography (ISCO®; 40 g SepaFlash® Silica Flash Column, petroleum ether/EtOAc with EtOAc from 0˜50%, 40 mL/min, 254 mn) to give 3,6-dichloro-N-methyl-1H-pyrazolo[3,4-d]pyrimidin-4-amine (Intermediate 183-b) as a white solid (2 g, 56.1% yield). ¹H NMR (400 MHz, CDCl₃) δ ppm 3.24 (d, J=5.0 Hz, 3H), 6.08 (br s, 1H), 11.50 (br s, 1H).

### Step 3: 3,6-dichloro-N-methyl-1-(2-trimethylsilylethoxymethyl)pyrazolo[3,4-d]pyrimidin-4-amine

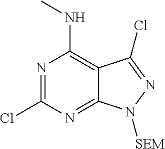 [SMILES: CNc1nc(Cl)nc2c1c(Cl)nn2COCC[Si](C)(C)C]

A mixture of 3,6-dichloro-N-methyl-1H-pyrazolo[3,4-d]pyrimidin-4-amine (Intermediate 183-b) (2 g, 9.17 mmol), Cs₂CO₃ (8.97 g, 27.5 mmol), SEM-Cl (1.84 g, 11.0 mmol) in THF (3 mL) was stirred at 25° C. for 16 hrs under N₂ atmosphere. The reaction was concentrated and the residue was purified by flash chromatography (ISCO®; 40 g SepaFlash® Silica Flash Column, petroleum ether/EtOAc with EtOAc from 0˜50%, 80 mL/min, 254 nm) to give 3,6-dichloro-N-methyl-1-(2-trimethylsilylethoxymethyl)-pyrazolo[3,4-d]pyrimidin-4-amine (Intermediate 183-c) as a white solid (2 g, 62.6% yield). ¹H NMR (400 MHz, DMSO-d₆) δ ppm −0.08 (s, 9H), 0.77-0.87 (m, 2H), 2.98 (br s, 3H), 3.53-3.62 (m, 2H), 5.39-5.55 (m, 2H), 7.87 (br d, J=3.7 Hz, 1H).

### Step 4: 1-[4-[[3-chloro-4-(methylamino)-1-(2-trimethylsilylethoxymethyl) pyrazolo [3,4-d]pyrimidin-6-yl]amino]indazol-1-yl]-2-methyl-propan-2-ol

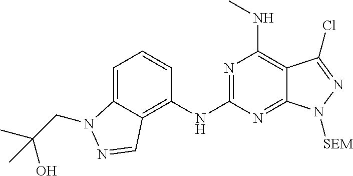 [SMILES: CNc1nc(Nc2cccc3c2cnn3CC(C)(C)O)nc2c1c(Cl)nn2COCC[Si](C)(C)C]

A mixture of 1-(4-aminoindazol-1-yl)-2-methylpropan-2-ol (Intermediate 97-b) (294 mg, 1.44 mmol), 3,6-dichloro-N-methyl-1-(2-trimethylsilylethoxymethyl)pyrazolo[3,4-d]pyrimidin-4-amine (Intermediate 183-c) (500 mg, 1.44 mmol), Pd₂(dba)₃ (131 mg, 0.143 nmol), Xantphos (166 mg, 0.287 mmol) and Cs₂CO₃ (1.17 g, 3.59 mmol) in toluene (5 mL) was degassed and purged with N₂ for 3 times, and then stirred at 100° C. for 16 hrs under N₂ atmosphere. The suspension was filtered and concentrated to give a residue, which was purified by flash chromatography (ISCO®; 12 g SepaFlash® Silica Flash Column, petroleum ether/EtOAc with EtOAc from 0˜500%, 20 mL/min, 254 mn) to give 1-[4-[[3-chloro-4-(methylamino)-1-(2-trimethylsilylethoxymethyl)pyrazolo[3,4-d]pyrimidin-6-yl]amino]indazol-1-yl]-2-methyl-propan-2-ol (Intermediate 183-d) as a yellow oil (550 ng, 74.1% yield). ¹H NMR (400 MHz, DMSO-d₆) δ ppm −0.15-0.07 (m, 9H), 0.72-0.95 (m, 2H), 1.12 (s, 6H), 3.04 (d, J=4.6 Hz, 3H), 3.53-3.65 (m, 2H), 4.26 (s, 2H), 4.62-4.73 (m, 1H), 5.46 (s, 2H), 7.17-7.25 (m, 1H), 7.26-7.37 (m, 2H), 7.86-8.03 (m, 1H), 8.42 (s, 1H), 9.49 (s, 1H).

### Step 5:1-[4-[[3-chloro-(hydroxymethyl)-4-(methylamino)pyrazolo[3,4-d]pyrimidin-6-yl]amino]indazol-1-yl]-2-methyl-propan-2-ol

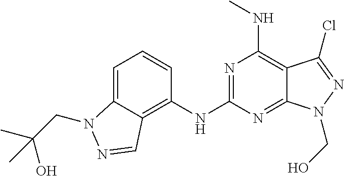 [SMILES: CNc1nc(Nc2cccc3c2cnn3CC(C)(C)O)nc2c1c(Cl)nn2CO]

A solution of 1-[4-[[3-chloro-4-(methylamino)-1-(2-trimethylsilylethoxymethyl)pyrazolo[3,4-d]pyrimidin-6-yl]amino]indazol-1-yl]-2-methyl-propan-2-ol (Intermediate 183-d) (200 mg, 0.386 mmol) in a mixed DCM (4 mL) and TFA (2 mL) was stirred at 25° C. for 1 h under N₂ atmosphere. The reaction mixture was concentrated to give 1-[4-[[3-chloro-1-(hydroxymethyl)-4-(methylamino)pyrazolo[3,4-d]pyrimidin-6-yl]amino]indazol-1-yl]-2-methyl-propan-2-ol (Intermediate 183-e) as a brown oil (150 mg, crude). LCMS (ESI) m/z 417.1 [M+H]⁺.

### Step 6: 1-[4-[[3-chloro-4-(methylamino)-1H-pyrazolo [3,4-d]pyrimidin-6-yl]amino]indazol-1-yl]-2-methyl-propan-2-ol

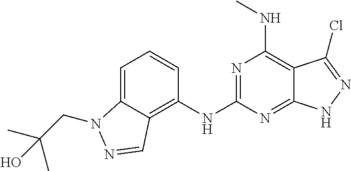 [SMILES: CNc1nc(Nc2cccc3c2cnn3CC(C)(C)O)nc2[nH]nc(Cl)c12]

A mixture of 1-[4-[[3-chloro-1-(hydroxymethyl)-4-(methylamino)pyrazolo[3,4-d]pyrimidin-6-yl]amino]indazol-1-yl]-2-methyl-propan-2-ol (Intermediate 183-e) (150 mg, 0.359 mmol) and LiOH·H₂O (151 mg, 3.60 mmol) in THF (4 mL) and H₂O (1 mL) was stirred at 25° C. for 1 hr under N₂ atmosphere. The reaction solution was concentrated. The residue was purified by pre-HPLC (column: Boston Green ODS 150×30 mm×5 μm; mobile phase: [water (HCl)-ACN]; B %: 15%-55%, 9 min) to give the title compound as a white solid (32.7 mg, 23.5% yield). ¹H NMR (400 MHz, DMSO-d₆) δ ppm 1.13 (s, 6H), 3.15 (br d, J=4.2 Hz, 3H), 4.27-4.35 (m, 2H), 7.29-7.43 (m, 2H), 7.91 (d, J=7.3 Hz, 1H), 8.06 (br s, 1H), 8.52 (s, 1H), 10.41 (br s, 1H), 13.62 (br s, 1H). LCMS (ESI) m/z 387.1 [M+H]⁺.

### Example 184

### 2-methyl-1-[4-[[6-(methylamino)-9H-purin-2-yl]amino]indazol-1-yl]propan-2-ol

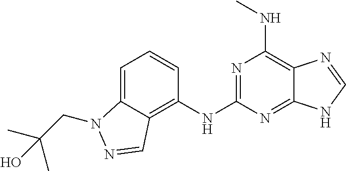 [SMILES: CNc1nc(Nc2cccc3c2cnn3CC(C)(C)O)nc2[nH]cnc12]

### Step 1: 2-chloro-N-methyl-9H-purin-6-amine

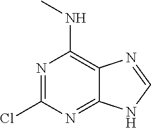

To a solution of 2,6-dichloro-91-H-purine (500 mg, 2.65 mmol) in EtOH-(5 mL) was added MeNH₂ (273 mg, 2.91 mmol, 33% purity). The mixture was stirred at 80° C. for 1 hr and then filtered. The filtrate was concentrated under reduced pressure to give a residue, which was triturated with MeOH at 25° C. for 10 min to give 2-chloro-N-methyl-9H-purin-6-amine (Intermediate 184-a) as a white solid (430 mg, 42.8% yield). LCMS (ESI) m/z 184.0 [M+4H].

### Step 2: 2-chloro-N-methyl-9-(2-trimethylsilylethoxymethyl)purin-6-amine

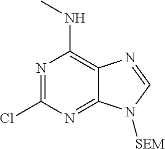 [SMILES: CNc1nc(Cl)nc2c1ncn2COCC[Si](C)(C)C]

To a solution of 2-choro-N-methyl-9H-purin-6-amine (Intermediate 184-a) (410 mg, 2.23 mmol) in MeCN (4 mL) was added SEM-Cl (446 mg, 2.68 mmol) and Cs₂CO₃ (1.46 g, 4.47 mmol). The mixture was stirred at 25° C. for 16 hrs and then filtered. The filtrate was concentrated under reduced pressure to give a residue, which was extracted with EtOAc (50 mL×3). The combined organic layers were washed with brine (50 mL×1), dried over Na₂SO₄, filtered, and concentrated under reduced pressure to give a residue. The residue was purified by flash silica gel chromatography (ISCO®; 4 g SepaFlash® Silica Flash Column, Eluent of 0˜25% Ethyl acetate/Petroleum ethergradient @ 50 mL/min) to give 2-chloro-N-methyl-9-(2-trimethylsilylethoxymethyl)purin-6-amine (Intermediate 184-b) as a white solid (440 mg, 47.1% yield). LCMS (ESI) m/z 314.1 [M+H]⁺.

### Step 3: 2-methyl-1-[4-[[6-(methylamino)-9-(2-trimethylsilylethoxymethyl) purin-2-yl]amino]indazol-1-yl]propan-2-ol

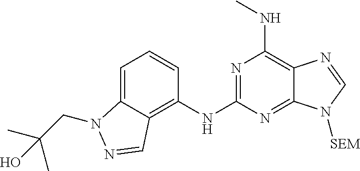 [SMILES: CNc1nc(Nc2cccc3c2cnn3CC(C)(C)O)nc2c1ncn2COCC[Si](C)(C)C]

A mixture of 1-(4-aminoindazol-1-yl)-2-methyl-propan-2-ol (Intermediate 97-b) (95.2 mg, 0.463 mmol), 2-chloro-N-methyl-9-(2-trimethylsilylethoxymethyl)purin-6-amine (Intermediate 184-b) (112 mg, 0.356 mmol), Pd₂(dba)₃ (32.6 mg, 35.6 μmol), Xantphos (413 mg, 71.3 μmol) and Cs₂CO₃ (290 mg, 0.892 mmol) in toluene (5 mL) was degassed and purged with N₂ for 3 times, then stirred at 100° C. for 16 hrs under N₂ atmosphere. The reaction mixture was filtered and the filtrate was concentrated. The residue was purified by flash chromatography (ISCO®; 4 g SepaFlash® Silica Flash Column, petroleum ether/EtOAc with EtOAc from 0˜50%, 12 mL/min, 254 nm) to give 2-methyl-1-[4-[[6-(methylamino)-9-(2-trimethylsilylethoxymethyl)purin-2-yl]amino]indazol-1-yl]propan-2-ol (Intermediate 184-c) as a yellow oil (100 mg, 58.1% yield). ¹HNMR (400 MHz, DMSO-d₆) δ ppm −0.09 (s, 9H), 0.82-0.90 (m, 2H), 1.12 (s, 6H), 3.02 (s, 3H), 3.55-3.66 (m, 2H), 4.25 (s, 2H), 4.61-4.74 (m, 1H), 5.47 (s, 2H), 7.14-7.28 (m, 2H), 7.60 (br s, 1H), 8.01 (s, 1H), 8.11 (d, J=7.5 Hz, 1H), 8.50 (s, 1H), 9.08 (s, 1H).

### Step 4: 2-methyl-1-[4-[[6-(methylamino)-9H-purin-2-yl]amino]indazol-1-yl]propan-2-ol

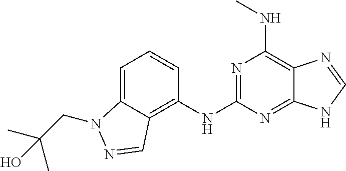 [SMILES: CNc1nc(Nc2cccc3c2cnn3CC(C)(C)O)nc2[nH]cnc12]

A solution of 2-methyl-1-[4-[[6-(methylamino)-9-(2-trimethylsilylethoxymethyl)purin-2-yl]amino]indazol-1-yl]propan-2-ol (Intermediate 184-c) (100 mg, 0.207 mmol) in TFA/DCM (6 mL, v/v=1:3) was stirred at 25° C. for 3 hrs under N₂ atmosphere. The reaction mixture was concentrated under reduced pressure. The residue was purified by pre-1-PLC (column: Boston Green ODS 150×30 mm×5 μm; mobile phase: [water (HCl)-ACN]; B %: 4%-44%, 9 min) to give the title compound as a white solid (12.0 mg, 16.4% yield). ¹H NMR (400 MHz, DMSO-d₆) δ ppm 1.13 (s, 6H), 3.12 (br s, 3H), 4.31 (s, 2H), 7.31-7.39 (m, 1H), 7.40-7.47 (m, 1H), 7.87 (br d, J=7.2 Hz, 1H), 8.36-8.55 (m, 2H), 9.37 (br s, 1H), 10.56 (br s, 1H); LCMS (ESI) m/z 353.4 [M+H]⁺.

### Example 185

### N⁶-(1-(2,2-difluoroethyl)-1H-indazol-4-yl)-N⁴-ethyl-3-methyl-1H-pyrazolo[3,4-d]pyrimidine-4,6-diamine

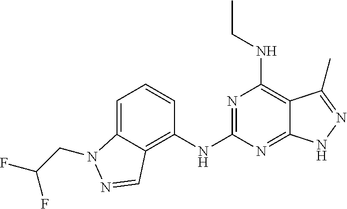 [SMILES: CCNc1nc(Nc2cccc3c2cnn3CC(F)F)nc2[nH]nc(C)c12]

### Step 1: 2-[(4,6-dichloro-3-methyl-pyrazolo[3,4-d]pyrimidin-1-yl)methoxy]ethyl-trimethyl-silane

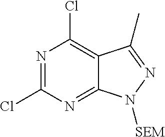 [SMILES: Cc1nn(COCC[Si](C)(C)C)c2nc(Cl)nc(Cl)c12]

To a solution of 4,6-dichloro-3-methyl-1H-pyrazolo[3,4-d] pyrimidine (1.7 g, 8.37 mmol) in MeCN (1 ml) was added SEM-Cl (1.40 g, 8.37 mmol) and Cs₂CO₃ (8.18 g, 25.1 mmol). The mixture was stirred at 25° C. for 16 hrs, diluted with H₂O (30 mL) and extracted with EtOAc (30 mL×3). The combined organic layers were washed with brine (30 mL×2), dried with anhydrous Na₂SO₄, filtered and concentrated under reduced pressure to give a residue. The residue was purified by flash silica gel chromatography (ISCO®; 4 g SepaFlash® Silica Flash Column, Eluent of 0˜7% petroleum ether/EtOAc @35 mL/min) to give 2-[(4,6-dichloro-3-methyl-pyrazolo[3,4-d]pyrimidin-1-yl)methoxy]ethyl-trimethyl-silane (Intermediate 185-a) as a white solid (1.3 g, 46.5% yield). LCMS (ESI) m/z 333.1 [M+H]⁺.

### Step 2: 6-chloro-N-ethyl-3-methyl-1-(2-trimethylsilylethoxymethyl)pyrazolo [3,4-d]pyrimidin-4-amine

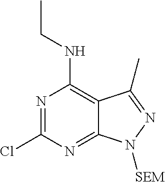 [SMILES: CCNc1nc(Cl)nc2c1c(C)nn2COCC[Si](C)(C)C]

To a solution of 2-[(4,6-dichloro-3-methyl-pyrazolo[3,4-d]pyrimidin-1-yl)methoxy]ethyl-trimethyl-silane (Intermediate 185-a) (600 mg, 1.80 mmol) in MeCN (1 mL) was added ethanamine; hydrochloride (254 mg, 2.16 mmol) and K₂CO₃ (746 mg, 5.40 mmol). The mixture was stirred at 25° C. for 16 hrs, diluted with H-0 (30 mL) and extracted with EtOAc (30 mL×3). The combined organic layers were washed with brine (30 mL×2), dried with anhydrous Na₂SO₄, filtered, and concentrated under reduced pressure to give a residue. The residue was purified by flash silica gel chromatography (ISCO®; 40 g SepaFlash® Silica Flash Column, Eluent of 0˜20% petroleum ether/EtOAc@ 35 mL/min) to provide 6-chloro-N-ethyl-3-methyl-1-(2-trimethylsilylethoxymethyl)pyrazolo[3,4-d]pyrimidin-4-amine (Intermediate 185-b) as a white solid (600 mg, 92.6% yield). LCMS (ESI) m/z 342.2 [M+H]⁺.

### Step 3: N⁶-[1-(2,2-difluoroethyl)indazol-4-yl]-N⁴-ethyl-3-methyl-1-(2-trimethylsilylethoxymethyl)pyrazolo[3,4-d]pyrimidine-4,6-diamine

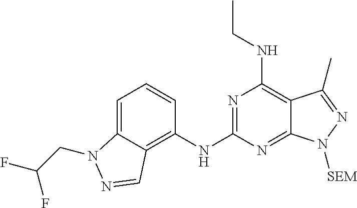 [SMILES: CCNc1nc(Nc2cccc3c2cnn3CC(F)F)nc2c1c(C)nn2COCC[Si](C)(C)C]

To a solution of 1-(2,2-difluoroethyl)-1H-indazol-4-amine (Intermediate 94-b) (57.7 mg, 0.292 nmol) in toluene (1 mL) was added 6-chloro-N-ethyl-3-methyl-1-(2-trimethylsilylethoxymethyl)pyrazolo[3,4-d]pyrimidin-4-amine (Intermediate 185-b) (100 mg, 0.292 mmol), Pd₂(dba)₃ (26.8 mg, 29.2 μmol), Xantphos (33.9 mg, 58.5 mol) and Cs₂CO₃ (95.3 mg, 292.5 □mol). The mixture was stirred at 100° C. for 16 hrs under N₂ atmosphere. The reaction mixture was purified by flash silica gel chromatography (ISCO®; 4 g SepaFlash® Silica Flash Column, Eluent of 0˜100% Ethyl acetate/Petroleum ether gradient @ 25 mL/min) to give N⁶-[1-(2,2-difluoroethyl)indazol-4-yl]-N⁴-ethyl-3-methyl-1-(2-trimethylsilylethoxymethyl)pyrazolo [3,4-d]pyrimidine-4,6-diamine (Intermediate 185-c) as a black oil (60 mg, 40.8% yield). ¹H NMR (400 MHz, DMSO-d₆) δ ppm 0.00 (s, 9H), 0.92-0.97 (m, 2H), 1.34 (t, J=7.07 Hz, 3H), 3.39-3.43 (n, 3H), 3.63-3.73 (m, 4H), 4.97 (td, J=15.20, 3.50 Hz, 2H), 5.54 (s, 2H), 6.37-6.68 (m, 1H), 7.13 (t, J=5.82 Hz, 1H), 7.34-7.45 (m, 2H), 8.21 (d, J=7.63 Hz, 1H), 8.67 (s, 1H), 9.39 (s, 1H); LCMS (ESI) m/z 503.3 [M+H]⁺.

### Step 4: [6-[[1-(2,2-difluoroethyl)indazol-4-yl]amino]-4-(ethylamino)-3-methyl-pyrazolo[3,4-d]pyrimidin-1-yl]methanol

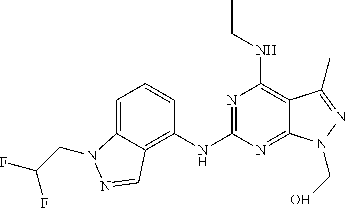 [SMILES: CCNc1nc(Nc2cccc3c2cnn3CC(F)F)nc2c1c(C)nn2CO]

A solution of N⁶-[1-(2,2-difluoroethyl)indazol-4-yl]-N⁴-ethyl-3-methyl-1-(2-trimethylsilylethoxymethyl)pyrazolo[3,4-d]pyrimidine-4,6-diamine (Intermediate 185-a) (60 mg, 0.119 mmol) in TFA/DCM (1 mL, v/v=1/3) was stirred at 25° C. for 2 hrs under N₂ atmosphere. The reaction solution was concentrated under reduced pressure to give [6-[[1-(2,2-difluoroethyl)indazol-4-yl]amino]-4-(ethylamino)-3-methyl-pyrazolo[3,4-d]pyrimidin-1-yl]methanol (Intermediate 185-b) as a black oil (30 mg, 62.5% yield), which was used into the next step without further purification. LCMS (ESI) m/Z 403.2 [M+H]⁺.

### Step 5: N⁶-(1-(2,2-difluoroethyl)-1H-indazol-4-yl)-N⁴-ethyl-3-methyl-1H-pyrazolo[3,4-d]pyrimidine-4,6-diamine

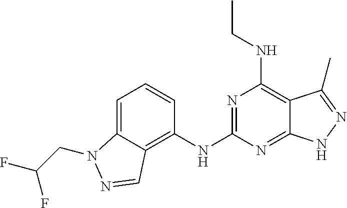 [SMILES: CCNc1nc(Nc2cccc3c2cnn3CC(F)F)nc2[nH]nc(C)c12]

A mixture of [6-[[1-(2,2-difluoroethyl)indazol-4-yl]amino]-4-(ethylamino)-3-methyl-pyrazolo[3,4-d]pyrimidin-1-yl]methanol (Intermediate 185-b) (30 mg, 74.6 μmol) and LiOH (8.93 mg, 0.373 mmol) in THF/H₂O (1 mL, v/v=3/1) was stirred at 25° C. for 2 hrs under N₂ atmosphere. The reaction mixture was concentrated under reduced pressure. The residue was purified by prep-HPLC (column: Boston Prime C18 150×30 mm×5 μm; mobile phase: [water (HC)-ACN]; B %: 11%-51%, 9 min) to give the title compound as a white solid (12 mg, 432% yield). ¹H NR (400 MHz, DMSO-d₆) δ ppm 1.12-1.30 (m, 3H), 2.62 (s, 3H), 3.58-3.62 (m, 2H), 4.86-5.05 (m, 2H), 6.16-6.69 (m, 1H), 730-7.59 (m, 2H), 7.80 (br s, 1H), 8.26-8.79 (m, 2H), 10.43 (br s, 1H), 14.03 (br s, 1H); LCMS (ESI) m/z 373.2 [M+H]⁺.

### Example 186

### N⁴-ethyl-N²-[1-(2-methylsulfonylethyl)indazol-4-yl]-5-(trifluoromethyl) pyrimidine-2,4-diamine

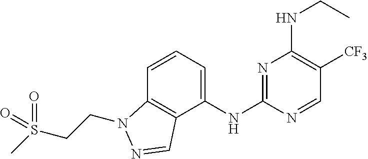 [SMILES: CCNc1nc(Nc2cccc3c2cnn3CCS(C)(=O)=O)ncc1C(F)(F)F]

A mixture of 1-(2-methylsulfonylethyl)indazol-4-amine (Intermediate 139-b) (30 mg, 0.125 mmol), 2-chloro-N-ethyl-5-(trifluoromethyl)pyrimidin-4-amine (Intermediate 69-a) (28.28 mg, 0.125 mmol) and HCl (1 M, 0.313 mL) in MeCN (2 mL) was degassed and purged with N₂ for 3 times, and then stirred at 80° C. for 16 hrs under N₂ atmosphere. The reaction mixture was concentrated under reduced pressure to remove MeCN. Then the residue was diluted with H₂O (5 mL) and neutralized to pH=7˜8 with 2M NaHCO₃, then extracted with EtOAc (10 mL×3). The combined organic layers were washed with brine (10 mL), dried with anhydrous Na₂SO₄, filtered and concentrated under reduced pressure to give a residue. The residue was purified by flash silica gel chromatography (ISCO®; 4 g SepaFlash® Silica Flash Column, Eluent of 0˜75% Ethyl acetate/Petroleum ether gradient @ 40 mL/min) to provide the title compound as a white solid (30.24 mg, 56.3%). ¹H NMR (400 MHz, DMSO-d₆) δ ppm 1.16 (s, 3H), 2.88 (s, 3H), 3.46-3.56 (m, 2H), 3.71-3.78 (m, 2H), 4.75-4.84 (m, 2H), 7.17-7.22 (m, 1H), 7.30-7.40 (m, 2H), 7.87-7.97 (m, 1H), 8.18 (s, 1H), 8.49 (s, 1H), 9.70 (s, 1H). ¹⁹F NMR (377 MHz, DMSO-d₆) δ ppm −60.31; LCMS (ESI) m/z 429.0 [M+H]⁺.

### Example 187

### N⁴-(cyclopropylmethyl)-N²-(1-(2-(methylsulfonyl)ethyl)-1H-indazol-4-yl)-5-(trifluoromethyl)pyrimidine-2,4-diamine

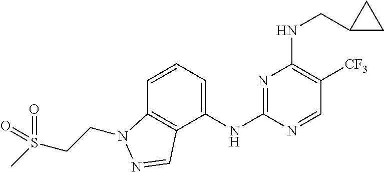 [SMILES: CS(=O)(=O)CCn1ncc2c(Nc3ncc(C(F)(F)F)c(NCC4CC4)n3)cccc21]

### Step 1: N-(4-chloro-5-(trifluoromethyl)pyrimidin-2-yl)-1-(2-(methylsulfonyl)ethyl)-1H-indazol-4-amine

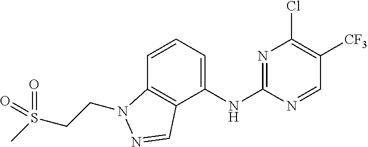 [SMILES: CS(=O)(=O)CCn1ncc2c(Nc3ncc(C(F)(F)F)c(Cl)n3)cccc21]

To a solution of 2,4-dichloro-5-(trifluoromethyl)pyrimidine (652 mg, 3.01 mmol) in t-BuOH (2 mL) and DCE (2 mL) was added ZnCl₂ (512 mg, 3.76 mmol) and 1-(2-methylsulfonylethyl)indazol-4-amine (Intermediate 139-b) (600 mg, 2.51 mmol). Then TEA (279 mg, 2.76 mmol) was added dropwise at 0° C. The reaction mixture was stirred at 25° C. for 3 hrs under N₂ atmosphere, diluted with H₂O (10 mL) and extracted with EtOAc (15 mL×3). The combined organic layers were washed with brine (10 mL), dried over Na₂SO₄, filtered and concentrated under reduced pressure. The residue was purified by flash chromatography (ISCO®; 12 g SepaFlash® Silica Flash Column, petroleum ether/EtOAc with EtOAc from 0˜80%, 60 mL/min, 254 nm/I₂/KMnO₄/PMA) to provide N-[4-chloro-5-(trifluoromethyl)pyrimidin-2-yl]-1-(2-methylsulfonylethyl)indazol-4-amine (Intermediate 187-a) as a white solid (450 mg, 40.2%). LCMS (ESI) m/z 441.9 [M+Na]⁺.

### Step 2: N⁴-(cyclopropylmethyl)-N²-(1-(2-(methylsulfonyl)ethyl)-1H-indazol-4-yl)-5-(trifluoromethyl)pyrimidine-2,4-diamine

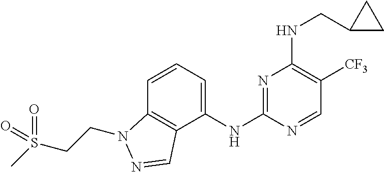 [SMILES: CS(=O)(=O)CCn1ncc2c(Nc3ncc(C(F)(F)F)c(NCC4CC4)n3)cccc21]

To a solution of N-[4-chloro-5-(trifluoromethyl)pyrimidin-2-yl]-1-(2-methylsulfonylethyl)indazol-4-amine (Intermediate 187-a) (50 mg, 0.119 mmol) in MeCN (1 mL) was added K₂CO₃ (41 mg, 0.297 mmol) and cyclopropylmethanamine (16 mg, 0.238 mmol). The mixture was degassed and purged with N₂ for three times, and stirred at 25° C. for 12 hrs under N₂ atmosphere. The reaction mixture was concentrated under reduced pressure. The residue was diluted with H₂O (10 mL) and extracted with EtOAc (15 mL×3). The combined organic layers were washed with brine (10 mL), dried over Na₂SO₄, filtered and concentrated under reduced pressure. The residue was purified by flash chromatography (ISCO®; 4 g SepaFlash® Silica Flash Column, petroleum ether/EtOAc with EtOAc from 0˜90%, 50 mL/min, 254 nm/I₂/KMnO₄/PMA). to provide the title compound as a white solid (50.17 mg, 90.5%). ¹H NMR (400 MHz, DMSO-d₆) δ (ppm 0.21-0.26 (m, 2H), 0.35-0.42 (m, 2H), 1.17 (br s, 1H), 2.90 (s, 3H), 3.31 (br s, 21H), 3.73 (t, J=6.80 Hz, 2H), 4.78 (t, J=6.80 Hz, 21H), 7.24 (s, 1H), 7.29-7.38 (m, 2H), 7.89 (d, J=6.8 Hz, 1H), 8.23 (s, 1H), 8.47 (s, 1H), 9.79 (s, 1H). ¹⁹F NMR (377 MHz, DMSO-d₆) δ ppm −60.31; LCMS (ESI) m/z 455.2 [M+H]⁺.

### Example 188

### N⁴-methyl-N²-[1-(2-methylsulfonylethyl) indazol-4-yl]-5-(trifluoromethyl) pyrimidine-2,4-diamine

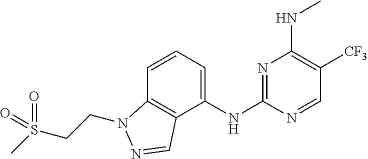 [SMILES: CNc1nc(Nc2cccc3c2cnn3CCS(C)(=O)=O)ncc1C(F)(F)F]

A mixture of 1-(2-methylsulfonylethyl)indazol-4-amine (Intermediate 139-b) (30 mg, 0.125 mmol), 2-chloro-N-methyl-5-(trifluoromethyl)pyrimidin-4-amine (Intermediate 1-a) (26.52 mg, 0.125 mmol) and HCl (1 M, 0.313 mL) in i-PrOH (2 mL) was degassed and purged with N₂ for 3 times, then stirred at 80° C. for 4 hrs under N₂ atmosphere. The reaction mixture was concentrated under reduced pressure, diluted with H₂O (5 mL), neutralized to pH=7˜8 with 2 M aq. NaHCO₃ and extracted with EtOAc (10 mL×3). The combined organic layers were washed with brine (10 mL), dried with anhydrous Na₂SO₄, filtered and concentrated under reduced pressure to give a residue, which was purified by flash silica gel chromatography (ISCO®; 4 g SepaFlash® Silica Flash Column, Eluent of 0˜75% Ethyl acetate/Petroleum ether gradient @40 mL/min) to provide the title compound as a white solid (12.74 mg, 24.5%). ¹H NMR (400 MHz, DMSO-d₆) δ ppm 2.89 (s, 3H), 2.93 (s, 3H), 3.71-3.77 (m, 2H), 4.74-4.85 (m, 2H), 7.15-7.22 (m, 1H), 7.29-7.41 (m, 2H), 7.86-8.01 (m, 1H), 8.27 (s, 1H), 8.56 (s, 1H), 9.76 (s, 1H). ¹⁹F NMR (377 MHz, DMSO-d₆) δ ppm −60.41; LCMS (ESI) m/z 415.0 [M+H]⁺.

### Example 189

### 2-(4-((4-(cyclopropylamino)-5-(trifluoromethyl)pyrimidin-2-yl)amino)-1H-pyrazolo[3,4-b]pyridin-yl)-2-methylpropanenitrile

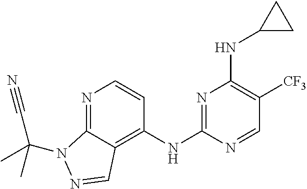 [SMILES: CC(C)(C#N)n1ncc2c(Nc3ncc(C(F)(F)F)c(NC4CC4)n3)ccnc21]

### Step 1: 2-(4-bromo-1H-pyrazolo[3,4-b]pyridin-1-yl)-2-methylpropanamide

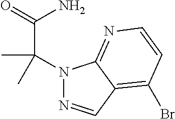 [SMILES: CC(C)(C(N)=O)n1ncc2c(Br)ccnc21]

A mixture of 4-bromo-1H-pyrazolo[3,4-b]pyridine (220 mg, 1.11 mmol), 2-bromo-2-methyl-propanamide (184 mg, 1.11 mmol) and Cs₂CO₃ (724 mg, 2.22 mmol) in DMF (2 mL) was degassed and purged with N₂ for 3 times, then stirred at 25° C. for 16 hrs under N₂ atmosphere. The reaction mixture was filtered and the filtrate was concentrated under reduced pressure to give a residue. The residue was diluted with H₂O (10 mL) and extracted with EtOAc (10 mL×3). The combined organic layers were washed with brine (20 mL), dried with anhydrous Na₂SO₄, filtered and concentrated under reduced pressure to give a residue. The residue was purified by flash silica gel chromatography (ISCO®; 4 g SepaFlash® Silica Flash Column, petroleum ether/EtOAc with EtOAc from 0˜60%, 40 mL/min, 254 nm) to provide 2-(4-bromopyrazolo[3,4-b]pyridin-1-yl)-2-methylpropenamide (Intermediate 189-a) as a white solid (660 mg, 93.0%). LCMS (ESI) m/z 283.0, 285.0 [M+H]⁺.

### Step 2: 2-(4-bromo-1H-pyrazolo[3,4-b]pyridin-1-yl)-2-methylpropanenitrile

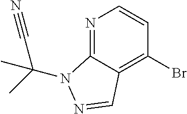

To a solution of 2-(4-bromopyrazolo[3,4-b]pyridin-1-yl)-2-methylpropanamide (Intermediate 189-a) (660 mg, 2.33 mmol) in DCM (5 mL) was added TEA (1.42 g, 14.0 mmol) and TFAA (1.47 g, 6.99 mmol). The mixture was stirred at 0-20° C. for 4 hrs under N₂ atmosphere, filtered and concentrated under reduced pressure to give a residue. The residue was diluted with DCM (5 mL) and extracted with H₂O (5 mL×3). The combined organic layers were washed with brine (5 mL), dried with anhydrous Na₂SO₄, filtered and concentrated under reduced pressure to give a residue. The residue was purified by flash silica gel chromatography (ISCO®; 4 g SepaFlash® Silica Flash Column, petroleum ether/EtOAc with EtOAc from 0-10%, 50 mL/min, 254 nm) to give 2-(4-bromopyrazolo[3,4-b]pyridin-1-yl)-2-methylpropanenitrile (Intermediate 189-b) as a white solid (230 mg, 98.0%). LCMS (ESI) m/z 265.0, 267.0 [M+H]⁺.

### Step 3: 2-(4-((diphenylmethylene)amino)-1H-pyrazolo[3,4-b]pyridin-1-yl)-2-methylpropanenitrile

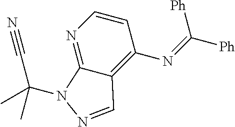 [SMILES: CC(C)(C#N)n1ncc2c(N=C(c3ccccc3)c3ccccc3)ccnc21]

A mixture of 2-(4-bromopyrazolo[3,4-b]pyridin-1-yl)-2-methyl-propanenitrile (Intermediate 189-b) (230 mug, 0.87 mmol), diphenylmethanimine (189 mg, 1.04 mmol), Cs₂CO₃ (678 mug, 2.08 mmol) and Xantphos Pd G4 (83.5 mg, 0.09 mmol) in dioxane (2 mL) was degassed and purged with N₂ for 3 times, and then stirred at 90° C. for 2 hrs under N₂ atmosphere. The reaction mixture was added filtered and concentrated under reduced pressure, diluted with 1-10 (10 mL) and extracted with EtOAc (5 mL×3). The combined organic layers were washed with brine (10 mL), dried with anhydrous Na₂SO₄, filtered and concentrated under reduced pressure to give a residue. The residue was purified by flash silica gel chromatography (ISCO®; 4 g SepaFlash® Silica Flash Column, petroleum ether/EtOAc with EtOAc from 0˜25%, 40 mL/min, 254 nm) to give 2-[4-(benzhydrylideneamino)pyrazolo[3,4-b]pyridin-1-yl]-2-methyl-propanenitrile (Intermediate 189-c) as a yellow oil (270 mg, 93.0%). LCMS (ESI) m/z 366.2 [M+H]⁺.

### Step 4: 2-(4-amino-1H-pyrazolo[3,4-b]pyridin-1-yl)-2-methylpropanenitrile

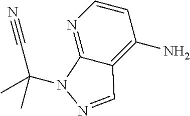

A HCl/dioxane (3 mL) solution of 2-[4-(benzhydrylideneamino)pyrazolo[3,4-b]pyridin-1-yl]-2-methyl-propanenitrile (Intermediate 189-c) (270 mug, 0.74 mmol) was stirred at 25° C. for 30 min under N₂ atmosphere. The reaction mixture was neutralized to pH=7˜8 with aq. NaHCO₃ (10 mL) and extracted with EtOAc (10 mL×3). The combined organic layers were washed with brine (20 mL), dried with anhydrous Na₂SO₄, filtered and concentrated under reduced pressure to give a residue. The residue was purified by flash silica gel chromatography (ISCO®; 4 g SepaFlash® Silica Flash Column, petroleum ether/EtOAc with EtOAc from 0-50%, 40 mil/min, 254 nm) to give 2-(4-aminopyrazolo[3,4-b]pyridin-1-yl)-2-methyl-propanenitrile (Intermediate 189-d) as a white solid (150 mg, 99.0%). LCMS (ESI) m/z 202.1 [M+H]⁺.

### Step 5: 2-(4-((4-(cyclopropylamino)-5-(trifluoromethyl)pyrimidin-2-yl)amino)-1H-pyrazolo[3,4-b]pyridin-1-yl)-2-methylpropanenitrile

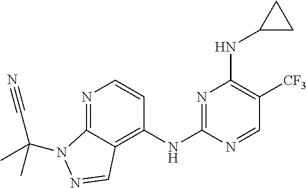 [SMILES: CC(C)(C#N)n1ncc2c(Nc3ncc(C(F)(F)F)c(NC4CC4)n3)ccnc21]

A mixture of 2-(4-aminopyrazolo[3,4-b]pyridin-1-yl)-2-methyl-propanenitrile (Intermediate 189-d) (50.0 mg, 0.25 mmol), 2-chloro-N-cyclopropyl-5-(trifluoromethyl)pyrimidin-4-amine (Intermediate 68-a) (59.0 mg, 0.25 mmol), bis(tri-tert-butylphosphine)palladium(0) (12.7 mg, 0.02 mmol) and Cs₂CO₃ (162 mg, 0.50 mmol) in DMF (1 mL) was taken up into a microwave tube. The sealed tube was heated at 120° C. for 1 hr under microwave. The reaction mixture was filtered and concentrated under reduced pressure to give a residue, which was diluted with 1-HO (10 mL) and extracted with EtOAc (10 mL×3). The combined organic layers were washed with brine (10 mL), dried with anhydrous Na₂SO₄, filtered and concentrated under reduced pressure to give a residue. The residue was purified by prep-HPLC (column: Phenomenex Synergi CIS 100×30 mm×4 um; mobile phase: [water(HCl)-ACN]; B %: 60%-90%, 8 min) to give the title compound as a white solid (15.82 mg, 99.9%). ¹H NMR (400 MHz, DMSO-d₆) δ ppm 0.68-0.77 (m, 2H), 0.83-0.93 (m, 2H), 2.16 (s, 6H), 2.93 (br s, 1H), 7.53 (s, 1H), 8.37 (s, 1H), 8.46 (br d, J=4 Hz, 1H), 8.55 (br d, J=4 Hz, 1H), 8.73 (s, 1H), 10.63 (s, 1H); ¹⁹F NMR (377 MHz, DMSO-d₆) δ ppm −60.64; LCMS (ESI) m/z 403.2 [M+H]⁺.

### Example 190

### N⁴-cyclopropyl-N²-(1-cyclopropyl-1H-indazol-4-yl)-5-(trifluoromethyl) pyrimidine-2,4-diamine

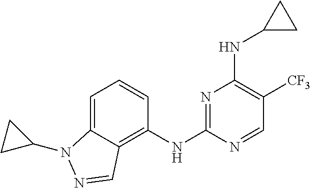 [SMILES: FC(F)(F)c1cnc(Nc2cccc3c2cnn3C2CC2)nc1NC1CC1]

### Step 1: 1-cyclopropyl-4-nitro-1H-indazole

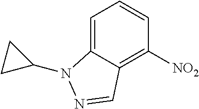 [SMILES: O=[N+]([O-])c1cccc2c1cnn2C1CC1]

To a solution of 4-nitro-1H-indazole (1 g, 6.13 nmol) in DCM (10 mL) was added cyclopropylboronic acid (1.05 g, 12.3 mmol), Cu(OAc)₂ (1.11 g, 6.13 mmol), Na₂CO₃ (1.30 g, 12.3 mmol) and pyridine (485 mg, 6.13 mmol). The mixture was degassed and purged with O₂ for three times, then stirred at 40° C. for 12 hrs under O₂ atmosphere (O₂ ballon). The residue was diluted with H₂O (20 mL) and extracted with EtOAc (25 mL×3). The combined organic layers were washed with brine (25 mL), dried over Na₂SO₄, filtered and concentrated under reduced pressure to give a residue. The residue was purified by flash chromatography (ISCO®; 40 g SepaFlash® Silica Flash Column, petroleum ether/EtOAc with EtOAc from 0˜40%, 50 mL/min, 254 nm) to give 1-cyclopropyl-4-nitro-indazole (Intermediate 190-a) as a brown solid (630 mg, 50.6% yield). ¹H NMR (400 MHz, CDCl₃) δ ppm 1.21-1.34 (m, 4H), 3.42-3.85 (m, 1H), 7.52 (t, J=8.0 Hz, 1H), 7.99 (d, J=8.4 Hz, 1H), 8.15 (d, J=7.6 Hz, 1H), 8.39 (s, 1H). LCMS (ESI) m/z 203.9 [M+H]⁺.

### Step 2: 1-cyclopropyl-1H-indazol-4-amine

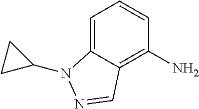 [SMILES: Nc1cccc2c1cnn2C1CC1]

To a solution of 1-cyclopropyl-4-nitro-indazole (Intermediate 190-a) (100 mg, 0.49 mmol) in EtOH (2 mL) was added Pd/C (10 mg, 0.49 mmol, 10% purity). The mixture was degassed and purged with H₂ for three times. The mixture was stirred at 25° C. for 1 hr under H₂ atmosphere (15 psi), filtered and concentrated under reduced pressure to give 1-cyclopropylindazol-4-amine (Intermediate 190-b) as a brown oil (90 mg, crude). LCMS (ESI) m/z 174.1 [M+H]⁺.

### Step 3: N⁴-cyclopropyl-N²-(1-cyclopropyl-1H-indazol-4-yl)-5-(trifluoromethyl) pyrimidine-2,4-diamine

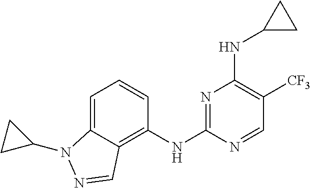 [SMILES: FC(F)(F)c1cnc(Nc2cccc3c2cnn3C2CC2)nc1NC1CC1]

To a solution of 1-cyclopropylindazol-4-amine (Intermediate 190-b) (90 mg, 0.52 mmol) in i-PrOH (2 mL) was added HCl (1M, 0.8 mL) and 2-chloro-N-cyclopropyl-5-(trifluoromethyl)pyrimidin-4-amine (Intermediate 68-a) (136 mg, 0.57 mmol). The mixture was stirred at 80° C. for 2 hrs. and then concentrated under reduced pressure to give a residue. The residue was purified by prep-HPLC (column: Phenomenex Synergi C18 100×30 mm×4 mm; mobile phase: [water (HCl)-ACN]; B %: 35%-65%, 8 min) to give the title compound as a gray solid (45.20 mg, 20.8% yield, 98% purity, HC salt). ¹H NMR (400 MHz, DMSO-d₆) δ ppm 0.71-0.77 (m, 2H), 0.78-0.86 (m, 2H), 1.03-1.14 (m, 4H), 2.81-2.99 (m, 1H), 3.72-3.79 (m, 1H), 7.42 (d, J=4.52 Hz, 2H), 7.94-8.24 (m, 2H), 8.39-8.58 (m, 2H), 10.63 (br s, 1H) ¹H NMR (377 MHz DMSO-d₆) δ ppm −60.89; LCMS (ESI) m/z 375.0 [M+H]⁺.

### Example 191

### N⁴-(1-methylcyclopropyl)-N²-(1-(2-(methylsulfonyl)ethyl)-1H-indazol-4-yl)-5-(trifluoromethyl)pyrimidine-2,4-diamine

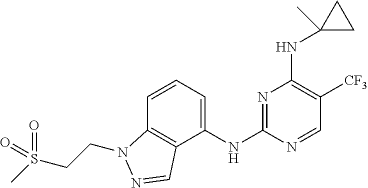 [SMILES: CC1(Nc2nc(Nc3cccc4c3cnn4CCS(C)(=O)=O)ncc2C(F)(F)F)CC1]

To a solution of N-[4-chloro-5-(trifluoromethyl)pyrimidin-2-yl]-1-(2-methylsulfonylethyl)indazol-4-amine (Intermediate 187-a) (50 mg, 0.119 mmol) in NMP (1 mL) was added Cs₂CO₃ (116 mg, 0357 mmol) and 1-methylcyclopropanamine (25 mg, 0.238 mmol, HCl salt). The mixture was degassed and purged with N₂ for three times, then stirred at 120° C. for 2 hrs under N₂ atmosphere. The reaction mixture was diluted with H₂O (10 mL) and extracted with EtOAc (15 mL×3). The combined organic layers were washed with brine (10 mL), dried over Na₂SO₄, filtered and concentrated under reduced pressure to give a residue, which was purified by prep-HPLC (column: Phenomenex Synergi C18 100×30 mm×4 mm; mobile phase: [water (HCl)-ACN]; B %: 25%-55%, 8 min as additive) to give the title compound as a white solid (21.35 mg, 36.5%). ¹H NMR (400 MHz, DMSO-d₆) δ ppm 0.74-0.81 (m, 2H), 0.86 (s, 2H), 1.41 (s, 3H), 2.90 (s, 3H), 3.74 (br t, J=6.8 Hz, 2H), 4.81 (t, J=6.8 Hz, 2H), 7.36-7.46 (m, 2H), 8.03 (br s, 1H), 8.32 (br d, J=6.8 Hz, 1H), 8.40 (br s, 1H), 8.60 (s, 1H), 10.45 (br s, 1H); 9F NMR (377 MHz, DMSO-d₆) δ ppm −60.64; LCMS (ESI) m/z 477.1 [M+Na]⁺.

### Example 192

### N⁴-cyclopropyl-5-(difluoromethyl)-N²-[1-(2-methylsulfonylethyl)indazol-4-yl]pyrimidine-2,4-diamine

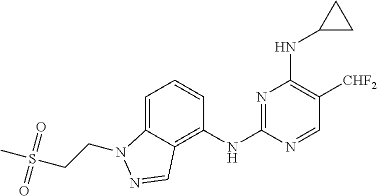 [SMILES: CS(=O)(=O)CCn1ncc2c(Nc3ncc(C(F)F)c(NC4CC4)n3)cccc21]

A mixture of 1-(2-methylsulfonylethyl)indazol-4-amine (Intermediate 139-b) (50 mg, 0.2 mmol), 2-chloro-N-cyclopropyl-5-(difluoromethyl)pyrimidin-4-amine (Intermediate 13-a) (50.4 mg, 0.2 mmol) and 1N HCl (0.30 mL) in i-PrOH (1 mL) was degassed and purged with N₂ for 3 times, then stirred at 80° C. for 1 hr under N₂ atmosphere. The reaction mixture was concentrated under reduced pressure to give a residue, which was triturated with MeOH (5 mL) to give the title compound as a white solid (16 mg, 17.6%). ¹H NMR (400 MHz, DMSO-d₆) δ ppm 0.72 (br d, J=2.8 Hz, 2H), 0.78-0.87 (m, 2H), 2.90 (s, 3H), 2.93 (br s, 1H), 3.75 (br t, J=6.8 Hz, 2H), 4.82 (t, J=6.8 Hz, 2H), 6.84-7.23 (m, 1H), 7.39-7.53 (m, 2H), 8.02 (br d, J=7.2 Hz, 1H), 8.28 (s, 1H), 8.49 (s, 2H), 10.76 (br s, 1H); LCMS (ESI) m/z 423.2 [M+H]⁺.

### Example 193

### N⁴-cyclopropyl-N²-(1-(2-morpholinoethyl)-1H-indazol-4-yl)-5-(trifluoromethyl)pyrimidine-2,4-diamine

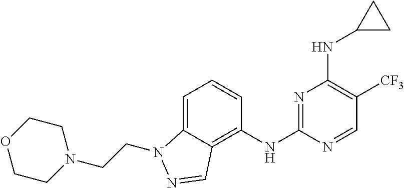 [SMILES: FC(F)(F)c1cnc(Nc2cccc3c2cnn3CCN2CCOCC2)nc1NC1CC1]

### Step 1: 4-(2-(4-nitro-1H-indazol-1-yl)ethyl)morpholine

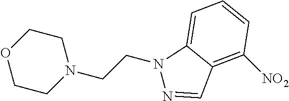 [SMILES: O=[N+]([O-])c1cccc2c1cnn2CCN1CCOCC1]

To a solution of 4-nitro-1H-indazole (700 mg, 4.29 mmol) in DMF (1 mL) was added Cs₂CO₃ (2.10 g, 6.414 nmol) and 4-(2-bromoethyl)morpholine (833 mg, 4.29 mmol). The mixture was stirred at 25° C. for 16 hrs and then filtered. The filtrate was concentrated under reduced pressure to give a residue. The residue was purified by flash chromatography (ISCO®; 20 g SepaFlash® Silica Flash Column, petroleum ether/EtOAc with EtOAc from 0˜50%, 40 mL/min, 254 nm) to provide 4-[2-(4-nitroindazol-1-yl)ethyl]morpholine (Intermediate 193-a) as a brown oil (840 mg, crude). LCMS (ESI) m/z 277.0 [M+H]^{T}.

### Step 2: 1-(2-morpholinoethyl)-1H-indazol-4-amine

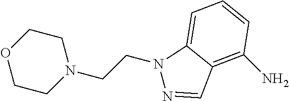 [SMILES: Nc1cccc2c1cnn2CCN1CCOCC1]

To a solution of 4-[2-(4-nitroindazol-1-yl)ethyl]morpholine (Intermediate 193-a) (840 mg, 3.04 mmol) in MeOH (10 mL) was added Pd/C (300 mg, 10% purity) The mixture was stirred at 25° C. for 16 hrs under H₂ (15 psi), and then filtered. The filtrate was concentrated under reduced pressure to give the crude product 1-(2-morpholinoethyl)indazol-4-amine (Intermediate 193-b) as a brown oil (690 mg, 92.1%), which was used into next step without purification. LCMS (ESI) m/z 247.2 [M+H]⁺.

### Step 3: N⁴-cyclopropyl-N²-(1-(2-morpholinoethyl)-1H-indazol-4-yl)-5-(trifluoromethyl)pyrimidine-2,4-diamine

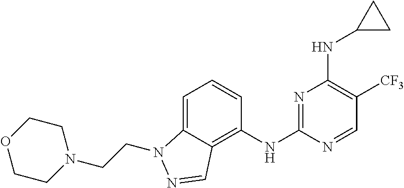 [SMILES: FC(F)(F)c1cnc(Nc2cccc3c2cnn3CCN2CCOCC2)nc1NC1CC1]

A mixture of 1-(2-morpholinoethyl)indazol-4-amine (Intermediate 193-b) (150 mg, 0.60 mmol), 2-chloro-N-cyclopropyl-5-(trifluoromethyl)pyrimidin-4-amine (Intermediate 68-a) (159 mg, 0.66 mmol) and HC (1 M, 0.91 mL) in i-PrOH (4 mL) was degassed and purged with N₂ for 3 times, and then stirred at 80° C. for 4 hr under N₂ atmosphere. The reaction mixture was diluted by H₂O (10 mL) and extracted with EtOAc (50 mL×3). The combined organic layers were washed with brine (20 ml), dried with anhydrous Na₂SO₄, filtered and concentrated under reduced pressure to give a residue. The residue was purified by prep-HPLC (column: 2 Phenomenex Gemini CIS 75×40 mm×3 um; mobile phase: [water(NH₃H₂O)-ACN]; B %: 38%-68%, 9.5 min) to provide the title compound as a white solid (107.56 mg, 38.1%). ¹H NMR (400 MHz, DMSO-d₆) δ ppm 0.63-0.72 (m, 2H), 0.73-0.82 (m, 2H), 2.42 (br s, 4H), 2.74 (t, J=6.64 Hz, 2H), 2.87 (br d, J=2.76 Hz, 1H), 3.45-3.54 (m, 4H), 4.47 (t, J=6.54 Hz, 2H), 7.21 (br s, 1H), 7.24-7.34 (m, 2H), 8.19 (br d, J=7.28 Hz, 1H), 8.25 (s, 1H), 8.47 (s, 1H), 9.84 (s, 1H). ¹⁹F NMR (377 MHz, DMSO-d₆) δ ppm −60.00; LCMS (ESI) m/z 448.1 [M+H]⁺.

### Example 194

### N⁴-cyclopropyl-N²-(1-(2-(isopropylsulfonyl)ethyl)-1-indazol-4-yl)-5-(trifluoromethyl)pyrimidine-2,4-diamine

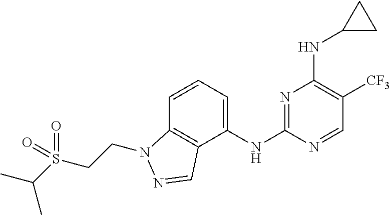 [SMILES: CC(C)S(=O)(=O)CCn1ncc2c(Nc3ncc(C(F)(F)F)c(NC4CC4)n3)cccc21]

### Step 1: 2-(4-nitro-1H-indazol-1-yl)ethan-1-ol

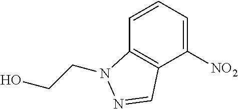 [SMILES: O=[N+]([O-])c1cccc2c1cnn2CCO]

To a solution of 4-nitro-1H-indazole (1 g, 6.13 mmol) in DMF (10 mL) was added 2-bromoethanol (919 mg, 7.36 mmol) and K₂CO₃ (1.69 g, 12.3 mmol). The mixture was stirred at 110° C. for 3 hrs.

The resulting mixture was diluted with H₂O (20 mL) and extracted with EtOAc (35 mL×3). The combined organic layers were washed with brine (30 mL), dried over Na₂SO₄, filtered and concentrated under reduced pressure to give a residue. The residue was purified by prep-HPLC (column: Phenomenex Synergi C18 100×30 mm×4 um; mobile phase: [water (HCl)-ACN]; B %: 15%-45%, 10 min) to give 2-(4-nitroindazol-1-yl)ethan-1-ol (Intermediate 194-a) as a yellow solid (400 mg, 31.5%). ¹H NMR (400 MHz, DMSO-d₆) δ ppm 3.83 (t, J=5.2 Hz, 2H), 4.04-4.41 (m, 1H), 4.58 (t, J=5.4 Hz, 2H), 7.61 (t, J=8.08 Hz, 1H), 8.15 (d, J=7.62 Hz, 1H), 8.24 (d, J=8.40 Hz, 1H), 8.51 (s, 1H).

### Step 2: 2-(4-nitro-1H-indazol-1-yl)ethyl 4-methylbenzenesulfonate

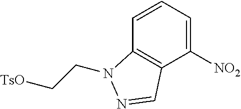 [SMILES: Cc1ccc(S(=O)(=O)OCCn2ncc3c([N+](=O)[O-])cccc32)cc1]

To a solution of 2-(4-nitroindazol-1-yl)ethan-1-ol (350 mg, 1.69 mmol) in DCM (5 mL) was added TEA (513 mg, 5.07 mmol) and TsCl (483 mg, 2.53 mmol). The mixture was stirred at 25° C. for 3 hrs. The residue was diluted with H₂O (20 mL) and extracted with DCM (25 mL×3). The combined organic layers were washed with brine (20 mL), dried over Na₂SO₄, filtered and concentrated under reduced pressure to give a residue. The residue was purified by flash chromatography (ISCO®; 12 g SepaFlash® Silica Flash Column, petroleum ether/EtOAc with EtOAc from 0-50%, 30 mL/min, 254 nm) to give compound 2-(4-nitroindazol-1-yl)ethyl 4-methylbenzenesulfonate (Intermediate 194-b) as a yellow solid (580 mg, 87.4%). LCMS (ESI) m/z 362.0 [M+H]⁺.

### Step 3: 1-(2-(isopropylthio)ethyl)-4-nitro-1H-indazole

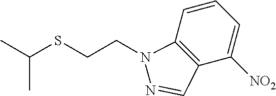 [SMILES: CC(C)SCCn1ncc2c([N+](=O)[O-])cccc21]

To a solution of 2-(4-nitroindazol-1-yl)ethyl 4-methylbenzenesulfonate (Intermediate 194-b) (200 mg, 0.55 mmol) in DMF (2 mL) was added isopropylsulfanylsodium (59.7 mg, 0.61 mmol) and the mixture was stirred at 25° C. for 1 hr. The reaction mixture was diluted with H₂O (10 mL) and extracted with EtOAc (15 mL×3). The combined organic layers were washed with brine (15 mL), dried over Na₂SO₄, filtered and concentrated under reduced pressure to give a residue. The residue was purified by flash chromatography (ISCO®; 12 g SepaFlash® Silica Flash Column, petroleum ether/EtOAc with EtOAc from 0-10%, 40 mL/min, 254 mn) to give compound 142-isopropylsulfanylethyl)-4-nitro-indazole (Intermediate 194-c) as a yellow oil (50 mg, 27.2%) LCMS (ESI) m/z 266.0 [M+H]⁺.

### Step 4: 1-(2-(isopropylsulfonyl)ethyl)-4-nitro-1H-indazole

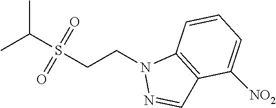 [SMILES: CC(C)S(=O)(=O)CCn1ncc2c([N+](=O)[O-])cccc21]

To a solution of 1-(2-isopropylsulfanylethyl)-4-nitro-indazole (Intermediate 194-c) (40 mg, 0.15 mmol) in THF (0.2 mL), H₂O (0.1 mL) and EtOH (0.2 mL) was added Oxone (185 mg, 0.3 mmol) and the mixture was stirred at 25° C. for 1 hr. The resulting mixture was diluted with H₂O (10 mL) and extracted with EtOAc (15 mL×3). The combined organic layers were washed with brine (15 mL), dried over Na₂SO₄, filtered and concentrated under reduced pressure to give a residue. The residue was purified by flash chromatography (ISCO®; 4 g SepaFlash® Silica Flash Column petroleum ether/EtOAc with EtOAc from 0-50%, 30 mL/min, 254 nm) to give compound 1-(2-isopropylsulfonylethyl)-4-nitro-indazole (Intermediate 194-d) as a yellow solid (40 mg, 89.2%). LCMS (ESI) m/z 297.9 [M+H]⁺.

### Step 5: 1-(2-(isopropylsulfonyl)ethyl)-1H-indazol-4-amine

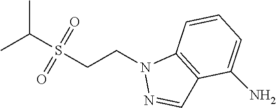 [SMILES: CC(C)S(=O)(=O)CCn1ncc2c(N)cccc21]

To a solution of 1-(2-isopropylsulfonylethyl)-4-nitro-indazole (Intermediate 194-d) (40 mg, 0.13 mmol) in EtOH (2 mL) was added Pd/C (20 mg, 0.09 mmol, 50% purity) and the mixture was degassed and purged with H₂ for three times. The mixture was stirred at 25° C. for 1 hr under H₂ (15 psi) atmosphere and then filtered. The filtrate was concentrated under reduced pressure to give compound 1-(2-isopropylsulfonylethyl)indazol-4-amine (Intermediate 194-e) as a yellow solid (30 mg, 83.4%). LCMS (ESI) m/z 268.1 [M+H]⁺.

### Step 6: N4-cyclopropyl-N²-(1-(2-(isopropylsulfonyl)ethyl)-1H-indazol-4-yl)-5-(trifluoromethyl)pyrimidine-2,4-diamine

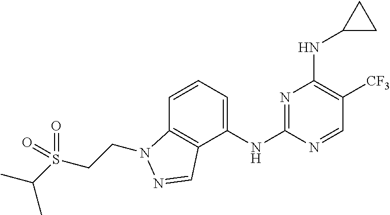 [SMILES: CC(C)S(=O)(=O)CCn1ncc2c(Nc3ncc(C(F)(F)F)c(NC4CC4)n3)cccc21]

To a solution of 1-(2-isopropylsulfonylethyl)indazol-4-amine (Intermediate 194-e) (30 mg, 0.11 mmol) in i-PrOH (1 mL) was added HCl (1 M, 0.4 mL) and 2-chloro-N-cyclopropyl-5-(trifluoromethyl)pyrimidin-4-amine (Intermediate 68-a) (29.3 mg, 0.12 mmol). The mixture was stirred at 80° C. for 2 hrs. and then filtered. The filtrate was concentrated under reduced pressure to give a residue. The residue was purified by prep-HPLC (column: Phenomenex Synergi C18 100×30 mm×4 um; mobile phase: [water (HCl)-ACN]; 13%: 25%-55%, 8 min) to give the title compound as a white solid (6.4 mg, 10.9%). H NMR (400 MHz, CD₃OD) δ ppm 0.70-0.76 (m, 2H), 0.77-0.85 (m, 2H), 1.27 (d, J=7.02 Hz, 6H), 2.87-2.94 (m, 1H), 2.98-3.08 (m, 1H), 3.72 (t, J=6.40 Hz, 2H), 4.93 (t, J=6.40 Hz, 2H), 7.46-7.54 (m, 1H), 7.55-7.61 (m, 1H), 7.67-7.77 (m, 1H), 8.24 (d, J=0.8 Hz, 1H). 8.25 (s, 1H); ¹⁹F NMR (377 MHz, CD₃OD) δ ppm −64.40; LCMS (ESI) m/z 469.1 [M+H]⁺.

### Example 195

### N⁴-cyclopropyl-N²-(1-(2-methyl-1-(methylsulfonyl)propan-2-yl)-1H-indazol-4-yl)-5-(trifluoromethyl)pyrimidine-2,4-diamine

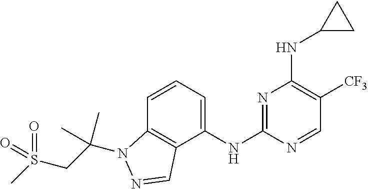 [SMILES: CC(C)(CS(C)(=O)=O)n1ncc2c(Nc3ncc(C(F)(F)F)c(NC4CC4)n3)cccc21]

### Step 1: methyl 2-methyl-2-(4-nitro-1H-indazol-1-yl)propanoate

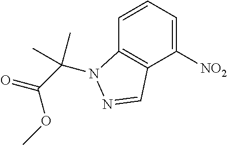 [SMILES: COC(=O)C(C)(C)n1ncc2c([N+](=O)[O-])cccc21]

To a solution of 4-nitro-1H-indazole (2.5 g, 15.3 mmol) and methyl 2-bromo-2-methyl-propanoate (3.33 g, 18.4 mmol) in DMF (20 mL) was added Cs₂CO₃ (5.99 g, 18.4 mmol). The mixture was stirred at 25° C. for 16 hrs and then filtered. The filtrate was concentrated under reduced pressure to give a residue. The residue was diluted by H₂O (100 mL) and extracted with EtOAc (150 mL×3). The combined organic layers were washed with brine (20 mL), dried with anhydrous Na₂SO₄, filtered and concentrated under reduced pressure to give a residue. The residue was purified by flash chromatography (ISCO®; 120 g SepaFlash® Silica Flash Column, petroleum ether/EtOAc with EtOAc from 0˜10%, 40 mL/min, 254 nm) to provide methyl 2-methyl-2-(4-nitroindazol-1-yl)propanoate (Intermediate 195-a) as a yellow solid (940 mg, 11.4%). ¹H NMR (400 MHz, DMSO-d₆) δ ppm 1.94 (s, 6H), 3.69 (s, 3H), 7.64 (t, J=8.12 Hz, 1H), 7.96 (d, J=8.50 Hz, 1H), 8.20 (d, J=7.76 Hz, 1H), 8.57 (s, 1H); LCMS (ESI) m/z 264.0 [M+H]⁺.

### Step 2: 2-methyl-2-(4-nitro-1H-indazol-1-yl)propan-1-ol

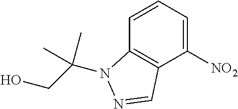 [SMILES: CC(C)(CO)n1ncc2c([N+](=O)[O-])cccc21]

To a solution of methyl 2-methyl-2-(4-nitroindazol-1-yl)propanoate (Intermediate 195-a) (1.19 g, 4.52 mmol) in THF (10 mL) was added LiAlH₄ (172 mg, 4.52 mmol) at 0° C. The mixture was stirred at 0-25° C. for 1 hr, quenched with saturated aq. NaCl (20 mL) and extracted with EtOAc (50 mL×3). The combined organic layers were washed with brine (20 mL), dried with anhydrous Na₂SO₄, filtered and concentrated under reduced pressure to give a residue. The residue was purified by flash chromatography (ISCO®; 12 g SepaFlash® Silica Flash Column, petroleum ether/EtOAc with EtOAc from 0˜50%, 40 mL/min, 254 nm) to give 2-methyl-2-(4-nitroindazol-1-yl)propan-1-ol (Intermediate 195-b) as a yellow oil (500 mg, 46.0%). LCMS (ESI) m/z 235.9 [M+H]⁺.

### Step 3: 2-(4-amino-1H-indazol-1-yl)-2-methylpropan-1-ol

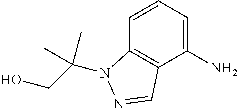

To a solution of 2-methyl-2-(4-nitroindazol-1-yl)propan-1-ol (Intermediate 195-b) (500 mg, 2.13 mmol) in MeOH (5 mL) was added Pd/C (200 mg, 10% purity). The mixture was stirred at 25° C. for 16 hrs under H₂ (15 psi), then filtered. The filtrate was concentrated under reduced pressure to give a crude product 2-(4-aminoindazol-1-yl)-2-methyl-propan-1-ol (Intermediate 195-c) as a brown oil (430 mg, 98.5%). LCMS (ESI) m/z 206.1 [M+H]⁺.

### Step 4: tert-butyl (1-(1-hydroxy-2-methylpropan-2-yl)-1H-indazol-4-yl)carbamate

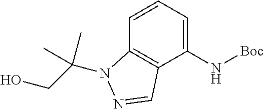 [SMILES: CC(C)(C)OC(=O)Nc1cccc2c1cnn2C(C)(C)CO]

To a solution of 2-(4-aminoindazol-1-yl)-2-methyl-propan-1-ol (Intermediate 195-c) (380 mg, 1.85 mmol) in THF (5 mL) was added Boc₂O (808 mg, 3.70 mmol). The mixture was stirred at 70° C. for 8 hrs and then concentrated under reduced pressure to give a residue. The residue was purified by flash chromatography (ISCO®; 4 g SepaFlash® Silica Flash Column, petroleum ether/EtOAc with EtOAc from 0-30%, 30 mL/min, 254 nm) to provide tert-butyl N-[1-(2-hydroxy-1,1-dimethyl-ethyl)indazol-4-yl]carbamate (Intermediate 195-d) as a white solid (450 mg, 79.3%). LCMS (ESI) m/z 306.2 [M+H]⁺.

### Step 5: 2-(4-((tert-butoxycarbonyl)amino)-1H-indazol-1-yl)-2-methylpropyl methanesulfonate

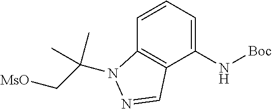 [SMILES: CC(C)(C)OC(=O)Nc1cccc2c1cnn2C(C)(C)COS(C)(=O)=O]

To a solution of tert-butyl N-[1-(2-hydroxy-1,1-dimethyl-ethyl)indazol-4-yl]carbamate (Intermediate 195-id) (350 mg, 1.15 mmol) in DCM (1 mL) was added TEA (231.95 ng, 2.29 mmol) and methanesulfonyl chloride (197 mg, 1.72 mmol) at 0° C. under N₂. The mixture was stirred at 0-25° C. for 1 hr, quenched by H₂O (10 mL) and extracted with EtOAc (50 mL×3). The combined organic layers were washed with brine (20 mL), dried with anhydrous Na₂SO₄, filtered and concentrated under reduced pressure to give a residue. The residue was purified by flash chromatography (ISCO®; 12 g SepaFlash® Silica Flash Column, petroleum ether/EtOAc with EtOAc from 0-30%, 40 mL/min, 254 nm) to provide [2-[4-(tert-butoxycarbonylamino)indazol-1-yl]-2-methyl-propyl]methanesulfonate (Intermediate 195-e) as a colorless oil (500 mg, 87.5%). LCMS (ESI) m/z 384.1 [M+H]⁺.

### Step 6: tert-butyl (1-(2-methyl-1-(methylthio)propan-2-yl)-1H-indazol-4-yl)carbamate

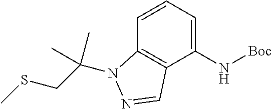 [SMILES: CSCC(C)(C)n1ncc2c(NC(=O)OC(C)(C)C)cccc21]

To a solution of [2-[4-(tert-butoxycarbonylamino)indazol-1-yl]-2-methyl-propyl]methanesulfonate (Intermediate 195-e) (250 mg, 0.65 mmol) in DMF (3 mL) was added sodium methanethiolate (91.4 mg, 1.30 mmol). The mixture was stirred at 120° C. for 2 hrs under microwave, then diluted with H₂O (10 mL) and extracted with EtOAc (50 ml×3). The combined organic layers were washed with brine (20 mL), dried with anhydrous Na₂SO₄, filtered and concentrated under reduced pressure to give a residue. The residue was purified by flash chromatography (ISCOR; 4 g SepaFlash® Silica Flash Column, petroleum ether/EtOAc with EtOAc from 0-30%, 30 mL/min, 254 nm) to give tert-butyl N-[1-(1,1-dimethyl-2-methylsulfanyl-ethyl)indazol-4-yl]carbamate (Intermediate 1954) as a colorless oil (90 mg, 29.3%). LCMS (ESI) n/z 336.2 [M+H]⁺.

### Step 7: tert-butyl (1-(2-methyl-1-(methylsulfonyl)propan-2-yl)-1H-indazol-4-yl)carbamate

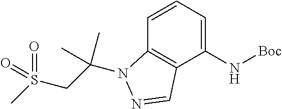 [SMILES: CC(C)(C)OC(=O)Nc1cccc2c1cnn2C(C)(C)CS(C)(=O)=O]

To a solution of tert-butyl N-[1-(1,1-dimethyl-2-methylsulfanyl-ethyl)indazol-4-yl]carbamate (Intermediate 195-f) (90 mg, 0.26 mmol) in i-PrOH (1 mL) and 1-120 (1 mL) was added potassium oxidooxy hydrogen sulfate (271 mg, 1.61 mmol). The mixture was stirred at 25° C. for 1 hr., and concentrated under reduced pressure. The residue was diluted with H₂O (5 mL) and extracted with EtOAc (30 mL×3). The combined organic layers were washed with brine (10 mL), dried with anhydrous Na₂SO₄, filtered and concentrated under reduced pressure to give a crude product tert-butyl N-[1-(1,1-dimethyl-2-methylsulfonyl-ethyl)indazol-4-yl]carbamate (Intermediate 195-g) as a brown oil (100 mg, 95.3%). LCMS (ESI) m/Z 368.2. [M+H]⁺.

### Step 8: 1-(2-methyl-1-(methylsulfonyl)propan-2-yl)-1H-indazol-4-amine

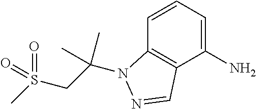 [SMILES: CC(C)(CS(C)(=O)=O)n1ncc2c(N)cccc21]

To a solution of tert-butyl N-[1-(1,1-dimethyl-2-methylsulfonyl-ethyl)indazol-4-yl]carbamate (Intermediate 195-g) (100 mg, 0.27 mmol) in MeOH (1 mL) was added 4M HCl in MeOH (1 mL, 4.0 mmol). The mixture was stirred at 25° C. for 1 hr. and then concentrated under reduced pressure to give a crude product 1-(1,1-dimethyl-2-methylsulfonyl-ethyl)indazol-4-amine (Intermediate 195-h) as a brown oil (80 mg, 96.7%). LCMS (ESI) m/z 268.1 [M+H]⁺.

### Step 9: N⁴-cyclopropyl-N²-(1-(2-methyl-1-(methylsulfonyl)propan-2-yl)-1H-indazol-4-yl)-5-(trifluoromethyl)pyrimidine-2,4-diamine

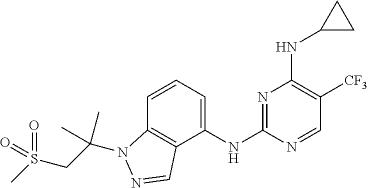 [SMILES: CC(C)(CS(C)(=O)=O)n1ncc2c(Nc3ncc(C(F)(F)F)c(NC4CC4)n3)cccc21]

A mixture of 1-(1,1-dimethyl-2-methylsulfonyl-ethyl)indazol-4-amine (Intermediate 195-h) (80 mg, 0.26 mmol, HCl salt), 2-chloro-N-cyclopropyl-5-(trifluoromethyl)pyrimidin-4-amine (Intermediate 68-a) (62.5 mg, 0.26 mmol) and 1N HCl (0.13 mL, 0.13 mmol) in i-PrOH (4 mL) was degassed and purged with N₂ for 3 times, and then stirred at 80° C. for 2 hrs under N₂ atmosphere. The reaction mixture was diluted by H₂O (10 mL) and extracted with EtOAc (50 mL×3). The combined organic layers were washed with brine (20 mL), dried with anhydrous Na₂SO₄, filtered and concentrated under reduced pressure to give a residue. The residue was purified by prep-HPLC (column: 2_Phenomenex Gemini CIS 75×40 mm×3 um; mobile phase: [water(NH₃H₂O)-ACN]; B %: 38%-68%, 9.5 min) to provide the title compound as a white solid (54.45 mg, 43.0%). ¹H NMR (400 MHz, CD₃OD) δ ppm 0.62-0.70 (m, 2H), 0.81-0.89 (m, 2H), 2.05 (s, 6H), 2.40 (s, 3H), 2.86 (tt, J=7.12, 3.69 Hz, 1H), 3.99 (s, 2H), 7.34-7.41 (nm, 1H), 7.55 (d, J=8.64 Hz, 1H), 8.14 (d, J=7.76 Hz, 1H), 8.17 (s, 1H), 8.26 (s, 1H); ¹⁹F NMR (377 MHz, CD₃OD) δ ppm −63.42; LCMS (ESI) m/z 4693 [M+H]⁺.

### Example 196

### N²-(1-tert-butylindazol-4-yl)-N⁴-cyclopropyl-5-(trifluoromethyl)pyrimidine-2,4-diamine

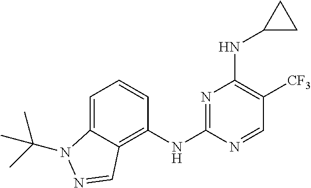 [SMILES: CC(C)(C)n1ncc2c(Nc3ncc(C(F)(F)F)c(NC4CC4)n3)cccc21]

### Step 1: N-[(2-fluoro-6-nitro-phenyl)methyleneamino]-2-methyl-propan-2-amine

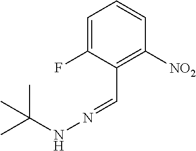 [SMILES: CC(C)(C)NN=Cc1c(F)cccc1[N+](=O)[O-]]

A mixture of 2-fluoro-6-nitro-benzaldehyde (1 g, 5.91 mmol) and tert-butylhydrazine (737 mg, 5.9 mmol, HCl salt) in EtOH (20 mL) was stirred at 20° C. for 2 hrs. The reaction mixture was diluted with NaHCO₃ aqueous solution (20 mL×3), extracted with EtOAc (50 mL×3) and filtered. The filtrate was dried over Na₂SO₄ and then concentrated under reduced pressure to give the crude product N-[(2-fluoro-6-nitro-phenyl)methyleneamino]-2-methyl-propan-2-amine (Intermediate 196-a) as a yellow solid (1.41 g, crude).

### Step 2: 1-tert-butyl-4-nitro-indazole

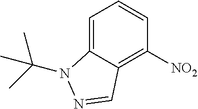 [SMILES: CC(C)(C)n1ncc2c([N+](=O)[O-])cccc21]

To a solution of N-[(2-fluoro-6-nitro-phenyl)methyleneamino]-2-methyl-propan-2-amine (Intermediate 196-a) (1.36 g, 5.68 mmol) in DMF (2 mL) was added NaH (273 mg, 6.8 mmol, 60% purity) at 0° C. Then the mixture was stirred at 80° C. for 1 hr., then quenched by H₂O (20 mL) and extracted with EtOAc (50 mL×3). The combined organic layers were washed with Brine (50 mL), dried over Na₂SO₄, filtered and concentrated under reduced pressure to give a residue. The residue was purified by column chromatography (ISCO®; 12 g SepaFlash® Silica Flash Column, petroleum ether/EtOAc with EtOAc from 0˜8%, 50 mL/min, 254 nm) to afford 1-tert-butyl-4-nitro-indazole (Intermediate 196-b) as a yellow solid (117 mg, 7.9%). LCMS (ESI) m/z 220.1 [M+H]⁺.

### Step 3: 1-tert-butylindazol-4-amine

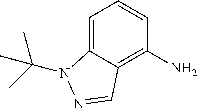

A mixture of 1-tert-butyl-4-nitro-indazole (Intermediate 196-b) (117 mg, 0.53 mmol) and Pd/C (17.6 mg, 53.4 μmol) in MeOH (4 mL) was degassed and purged with H₂ for 3 times, and then stirred at 25° C. for 2 hrs under H₂ atmosphere (15 psi). The reaction mixture was then quenched by H₂O (5 mL), extracted with EtOAc (10 mL×3) and filtered. The organic layers were concentrated under reduced pressure to give the crude product 1-tert-butylindazol-4-amine (Intermediate 196-c) as a yellow solid (96 mg, crude).

### Step 4N²-(1-tert-butylindazol-4-yl)-N⁴-cyclopropyl-5-(trifluoromethyl)pyrimidine-2,4-diamine

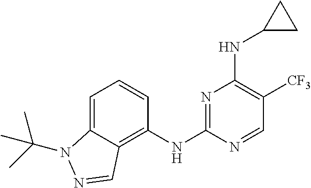 [SMILES: CC(C)(C)n1ncc2c(Nc3ncc(C(F)(F)F)c(NC4CC4)n3)cccc21]

A mixture of 1-tert-butylindazol-4-amine (Intermediate 196-c) (96 mg, 0.51 mmol), 2-chloro-N-cyclopropyl-5-(trifluoromethyl)pyrimidin-4-amine (Intermediate 68-a) (121 mg, 0.51 mmol) and HCl (1 M, 0.76 mL) in i-PrOH (3 mL) was degassed and purged with N₂ for 3 times, and then stirred at 80° C. for 2 hrs under N₂ atmosphere. The reaction mixture was diluted with NaHCO₃ aqueous solution (10 mL) and extracted with EtOAc (50 mL×3). The filtrate was dried over Na₂SO₄ and concentrated under reduced pressure to give a residue. The residue was purified by column chromatography (SiO2, Petroleum ether/Ethyl acetate=5:1) to afford the title compound as a white solid (90 ng, 44.7%). ¹H NMR (400 MHz, DMSO-d₆) δ ppm 0.65-0.70 (m, 2H) 0.75-0.80 (m, 2H) 1.69 (s, 9H) 2.86 (dt, J=7.2, 3.51 Hz, 1H) 7.22 (s, 1H) 7.27 (t, J=8 Hz, 1H) 7.45 (d, J=8 Hz, 1H) 8.17 (br d, J=7.6 Hz, 1H) 8.24 (s, 1H) 8.42 (s, 1H) 9.79 (s, 1H). ¹⁹F NMR (376 MHz, DMSO-d₆) δ ppm −60.00; LCMS (E/SI) m/z 391.1 [M+H]⁺.

### Example 197

### N⁴-cyclopropyl-N²-(1-(oxetan-3-ylmethyl)-1H-indazol-4-yl)-5-(trifluoromethyl) pyrimidine-2,4-diamine

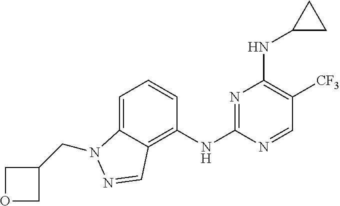 [SMILES: FC(F)(F)c1cnc(Nc2cccc3c2cnn3CC2COC2)nc1NC1CC1]

### Step 1: 4-nitro-1-(oxetan-3-ylmethyl)-1H-indazole

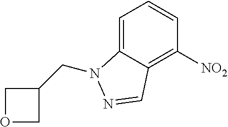 [SMILES: O=[N+]([O-])c1cccc2c1cnn2CC1COC1]

A mixture of 4-nitro-1H-indazole (300 ng, 1.84 nmol), 3-(bromomethyl)oxetane (278 mg, 1.84 mmol) and Cs₂CO₃ (1.20 g, 3.68 mmol) in DMF (5 mL) was degassed and purged with N₂ for 3 times, and then stirred at 25° C. for 2 hrs under N₂ atmosphere. The reaction mixture was filtered and the filtrate was concentrated under reduced pressure to give a residue. The residue was diluted with H₂O (30 mL) and extracted with EtOAc (10 mL×3). The combined organic layers were washed with brine (20 mL), dried with anhydrous Na₂SO₄, filtered and concentrated under reduced pressure. The resulting residue was purified by flash silica gel chromatography (ISCO®; 4 g SepaFlash® Silica Flash Column, petroleum ether/EtOAc with EtOAc from 0˜30%, 40 mL/min, 254 nm) to give compound 4-nitro-1-(oxetan-3-ylmethyl)indazole (Intermediate 197-a) as a yellow oil (400 ng, 95.0%). LCMS (ESI) m/z 234.0 [M+H]⁺.

### Step 2: 1-(oxetan-3-ylmethyl)-1H-indazol-4-amine

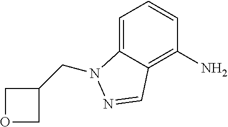 [SMILES: Nc1cccc2c1cnn2CC1COC1]

A mixture of 4-nitro-1-(oxetan-3-ylmethyl)indazole (Intermediate 197-a) (400 mg, 1.72 mmol), Fe (958 mg, 17.15 mmol) and NH₄Cl (917 mg, 17.2 mmol) in EtOH (4 mL) and H₂O (2 mL) was degassed and purged with N₂ for 3 times, and then stirred at 50° C. for 2 hrs under N₂ atmosphere. The reaction mixture was filtered and concentrated under reduced pressure to give a residue. The residue was diluted with H₂O (10 mL) and extracted with EtOAc (10 mL×3). The combined organic layers were washed with brine (20 mL), dried with anhydrous Na₂SO₄, filtered and concentrated under reduced pressure to give a residue. The resulting residue was purified by flash silica gel chromatography (ISCO®; 4 g SepaFlash® Silica Flash Column, petroleum ether/EtOAc with EtOAc from 0˜80%, 40 mL/min, 254 nm) to give compound 1-(oxetan-3-ylmethyl)indazol-4-amine (Intermediate 197-b) as a red oil (160 mg, 99.0%). ¹H NMR (400 MHz, DMSO-d₆) δ ppm 3.42-3.46 (m, 1H), 4.44 (m, J=4.0 Hz, 2H), 4.53 (d, J=8.4 Hz, 2H), 4.61 (dd, J=8.4 Hz, 2H), 5.77 (s, 2H), 6.14 (d, J=8.0 Hz, 1H), 6.71 (d, J=8.0 Hz, 1H), 7.02 (t, J=8.4 Hz, 1H), 8.06 (d, 1H); LCMS (ESI) m/z 204.1 [M+H]⁺.

### Step 3: N⁴-cyclopropyl-N²-(1-(oxetan-3-ylmethyl)-1H-indazol-4-yl)-5-(trifluoromethyl) pyrimidine-2,4-diamine

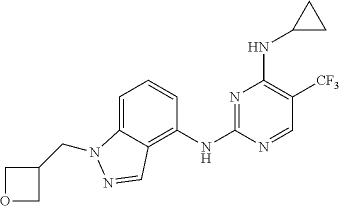 [SMILES: FC(F)(F)c1cnc(Nc2cccc3c2cnn3CC2COC2)nc1NC1CC1]

A mixture of 1-(oxetan-3-ylmethyl)indazol-4-amine (Intermediate 197-b) (160 mg, 0.79 mmol), 2-chloro-N-cyclopropyl-5-(trifluoromethyl)pyrimidin-4-amine (Intermediate 68-a) (187 mg, 0.79 nmol) and HCl (1 M, 0.79 mL) in i-PrOH (1 mL) was degassed and purged with N₂ for 3 times, and then stirred at 80° C. for 4 hrs under N₂ atmosphere. The reaction mixture was neutralized to pH=7˜8 by NaHCO₃ (10 mL) and extracted with EtOAc (10 mL×3). The combined organic layers were washed with brine (20 mL), dried with anhydrous Na₂SO₄, filtered and concentrated under reduced pressure to give a residue. The residue was purified by flash silica gel chromatography (ISCO®; 4 g SepaFlash® Silica Flash Column, DCM/MeOH with EtOAc from 0-20%, 40 mL/min, 254 nm) to give the title compound as a white solid (16.33 mg, 98.7%). ¹H NMR (500 MHz, CD₃OD) δ ppm 0.58-0.70 (m, 2H), 0.77-0.88 (m, 2H), 2.80-2.88 (m, 1H), 3.64-3.74 (m, 1H), 3.85 (d, J=8.0 Hz, 2H), 4.54-4.59 (m, 1H), 4.74-4.83 (m, 2H), 4.99 (br d, J=8.0 Hz, 1H), 7.41 (d, J=8.0 Hz, 1H), 7.79 (t, J=8.0 Hz, 1H), 8.23 (s, 1H), 8.38 (d, J=8.0 Hz, 1H), 9.16 (s, 1H), ¹⁹F NMR (471 MHz, CD₃OD) δ ppm −63.68; LCMS (ESI) m/z 405.1 [M+H]⁺.

### Examples 198 and 199

### N⁴-((1S,2R)-2-fluorocyclopropyl)-N²-(1-(2-(methylsulfonyl)ethyl)-1H-indazol-4-yl)-5-(trifluoromethyl)pyrimidine-2,4-diamine (Example 198) and N⁴-((1S,2S)-2-fluorocyclopropyl)-N²-(1-(2-(methylsulfonyl)ethyl)-1-indazol-4-yl)-5-(trifluoromethyl)pyrimidine-2,4-diamine (Example 199)

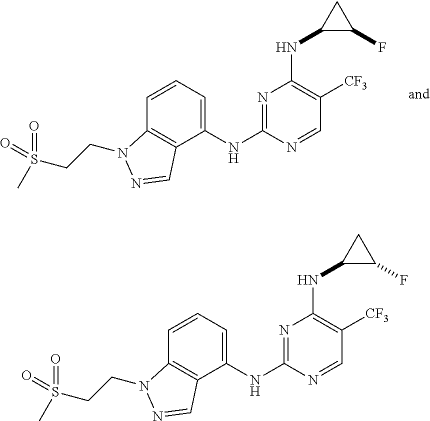

### Step 1: 2-chloro-N-(2-fluorocyclopropyl)-5-(trifluoromethyl)pyrimidin-4-amine

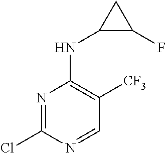 [SMILES: FC1CC1Nc1nc(Cl)ncc1C(F)(F)F]

To a solution of 2-fluorocyclopropanamine (210 mg, 1.88 mmol, HCl salt) in MeCN (2 mL) was added K₂CO₃(780 mg, 5.65 mmol) and 2,4-dichloro-5-(trifluoromethyl)pyrimidine (408 mg, 1.88 mmol). The mixture was degassed and purged with N₂ for three times, then stirred at 0° C. for 2 hrs under N₂ atmosphere. The resulting mixture was diluted with H₂O (10 mL) and extracted with EtOAc (15 mL×3). The combined organic layers were washed with brine (10 mL), dried over Na₂SO₄, filtered and concentrated under reduced pressure to give a residue. The residue was purified by flash chromatography (ISCO®, 12 g SepaFlash® Silica Flash Column, petroleum ether/EtOAc with EtOAc from 0˜10%, 50 mL/min, 254 nm/I₂/KMnO₄/PMA) to provide 2-chloro-N-(2-fluorocyclopropyl)-5-(trifluoromethyl)pyrimidin-4-amine (Intermediate 198-a) as a colorless oil (160 mg, 30.6%). LCMS (ESI) m/z 255.8 [M+H]⁺.

### Step 2: N⁴-((1S,2R)-2-fluorocyclopropyl)-N²-(1-(2-(methylsulfonyl)ethyl)-1H-indazol-4-yl)-5-(trifluoromethyl)pyrimidine-2,4-diamine (Example 198) and N⁴-((1S,2S)₂-fluorocyclopropyl)-N²-(1-(2-(methylsulfonyl)ethyl)-1H-indazol-4-yl)-5-(trifluoromethyl)pyrimidine-2,4-diamine (Example 199)

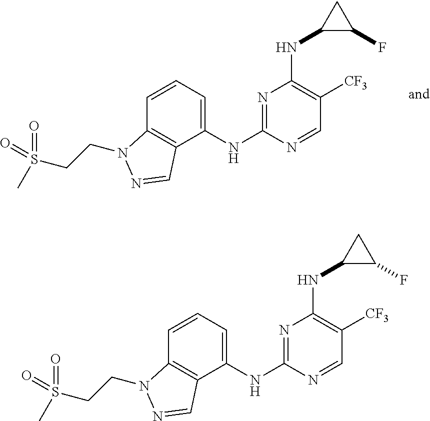

To a solution of 1-(2-methylsulfonylethyl)-1H-indazol-4-amine (Intermediate 139-b) (149 mg, 0.625 mmol) and 2-chloro-N-(2-fluorocyclopropyl)-5-(trifluoromethyl)pyrimidin-4-amine (Intermediate 198-a) (160 mg, 0.625 mmol) in i-PrOH (1 mL) was added 1N HCl (0.93 mL, 0.93 mmol). The mixture was stirred at 80° C. for 3 hrs., diluted with H₂O (30 mL) and extracted with EtOAc (30 mL×3). The combined organic layers were washed with brine (30 mL×2), dried with anhydrous Na₂SO₄, filtered and concentrated under reduced pressure to give a residue. The residue was purified by prep-HPLC (column: Phenomenex Synergi C18 100×30 mm×4 mm; mobile phase: [water(HCl)-ACN]; B %: 20%-50%, 10 min) to provide cis and trans isomers.

Fraction A (22.68 mg, RT=2.99 min, @ (Column: Xbridge C18 2.1×50 mm×5 □m; mobile phase: [water (HCl)-ACN]; B %: 20%-50%, 10 min) was tentatively assigned as compound Example 198: Compound N⁴-((1 S,2R)-2-fluorocyclopropyl)-N²-(1-(2-(methylsulfonyl)ethyl)-1H-indazol-4-yl)-5-(trifluoromethyl)pyrimidine-2,4-diamine was obtained as a white solid (22.68 mg, 20.9%). ¹H NMR (400 MHz, DMSO-d₆) δ ppm 1.15-1.25 (m, 1H), 1.36-1.46 (m, 1H), 2.86 (br d, J7=4.0 Hz, 1H), 2.90 (s, 3H), 3.74 (br t, J=6.8 Hz, 2H), 4.70-4.74 (m, 3H), 7.39-7.47 (m, 2H), 778 (br s, 1H), 8.06 (d, J=7.2 Hz, 1H), 8.44 (s, 1H), 8.50 (s, 1H), 10.48 (br s, 1H); ¹⁹F NMR (377 MHz, DMSO-d₆) δ (ppm −225.57, −60.49; LCMS (ESI) m/z 459.1 [M+H]⁺.

Fraction B (17.42 mg, RT=3.02 min, @ (Column: Xbridge C18 2.1×50 mm×5 □m; mobile phase: [water (HCL)-ACN]; 13%: 20%-50%, 10 min) was tentatively assigned as compound Example 199: Compound N⁴-((1S,2S)-2-fluorocyclopropyl)-N²-(1-(2-(methylsulfonyl)ethyl)-1H-indazol-4-yl)-5-(trifluoromethyl)pyrimidine-2,4-diamine was obtained as a white solid (17.42 mg, 15.65%). ¹H NMR (400 MHz, DMSO-d₆) δ ppm 1.19-1.29 (m, 1H), 1.37-1.52 (m, 1H), 2.90 (s, 3H), 3.29 (br s, 1H), 3.73 (br t, J=6.8 Hz, 2H), 4.72-4.92 (m, 3H), 7.29-7.41 (m, 2H), 7.59 (br s, 1H), 8.01-8.24 (m, 1H), 8.38 (s, 1H), 8.55 (s, 1H), 10.30 (br s, 1H); ¹⁹F NMR (377 MHz, DMSO-d₆) δ ppm −208.35, −60.34; LCMS (ESI) m/z 459.1 [M+H]⁺.

### Example 200

### 3-[4-[[4-(cyclopropylamino)-5-(trifluoromethyl)pyrimidin-2-yl]amino]indazol-1-yl]-2,2-dimethyl-propanenitrile

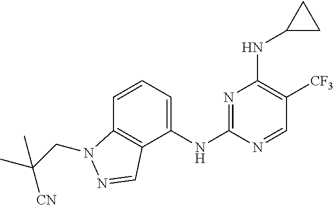 [SMILES: CC(C)(C#N)Cn1ncc2c(Nc3ncc(C(F)(F)F)c(NC4CC4)n3)cccc21]

### Step 1: 2,2-dimethyl-3-(4-nitroindazol-1-yl)propanenitrile

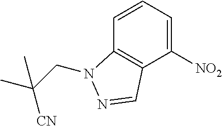 [SMILES: CC(C)(C#N)Cn1ncc2c([N+](=O)[O-])cccc21]

A mixture of 3-hydroxy-2,2-dimethyl-propanenitrile (300 mg, 3.03 mmol), 4-nitro-1H-indazole (518 mg, 3.18 mmol), 2-(tributyl-λ⁵-phosphanylidene)acetonitrile (876 mg, 3.63 mmol) in toluene (4 mL) was degassed and purged with N₂ for 3 times, and then stirred at 120° C. for 1 hr under N₂ atmosphere. The reaction mixture was concentrated under reduced pressure to give a residue, which was purified by column chromatography (ISCO®; 12 g SepaFlash® Silica Flash Column, petroleum ether/EtOAc with EtOAc from 0˜18%, 50 mL/min, 254 min) to afford 2,2-dimethyl-3-(4-nitroindazol-1-yl)propanenitrile (Intermediate 200-a) as a yellow solid (598 mg, 42.0%). LCMS (ESI) m/z 245.1 [M+H]⁺.

### Step 2: 3-(4-aminoindazol-1-yl)-2,2-dimethyl-propanenitrile

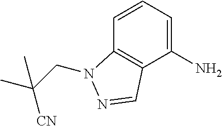 [SMILES: CC(C)(C#N)Cn1ncc2c(N)cccc21]

A mixture of 2,2-dimethyl-3-(4-nitroindazol-1-yl)propanenitrile (Intermediate 200-a) (702 mg, 2.9 mmol), Pd/C (94.8 mg, 0.29 mmol, 10% purity) in MeOH (10 ml) was degassed and purged with H₂ for 3 times, and then stirred at 25° C. for 2 hrs under H₂ atmosphere (15 psi). The reaction mixture was filtered and the filtrate was concentrated under reduced pressure to give a residue. The residue was purified by column chromatography (ISCO®; 12 g SepaFlash® Silica Flash Column, petroleum ether/EtOAc with EtOAc from 0-40%, 50 mL/min, 254 nm) to afford 3-(4-aminoindazol-1-yl)-2,2-dimethyl-propanenitrile (Intermediate 200-b) as a white solid (140 mg, 21.6%). ¹H NMR (400 MHz, DMSO-d₆) δ ppm 1.35 (s, 6H), 4.46 (s, 2H), 5.80 (s, 2H), 6.16 (d, J=7.2 Hz, 1H), 6.83 (d, J=8.0 Hz 1H), 7.00-7.08 (m, 1H), 8.15 (d, J=7.2 Hz, 1H); LCMS (ESI) m/z 215.1 [M+H]⁺.

### Step 3: 3-[4-[[4-(cyclopropylamino)-5-(trifluoromethyl)pyrimidin-2-yl]amino]indazol-1-yl]-2,2-dimethyl-propanenitrile

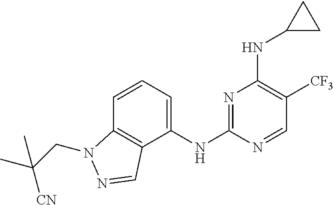 [SMILES: CC(C)(C#N)Cn1ncc2c(Nc3ncc(C(F)(F)F)c(NC4CC4)n3)cccc21]

To a solution of 3-(4-aminoindazol-1-yl)-2,2-dimethyl-propanenitrile (Intermediate 200-b) (50 mg, 0.23 mmol) and 2-chloro-N-cyclopropyl-5-(trifluoromethyl)pyrimidin-4-amine (Intermediate 68-a) (55.5 mg, 0.23 mmol) in i-PrOH (2 mL) was added 1N HCl (0.35 mL, 0.35 mmol). The reaction mixture was stirred at 80° C. for 1 hr, diluted with NaHCO₃ aqueous solution (2 mL), extracted with EtOAc (20 mL×3) and filtered, the filtrate was then dried over Na₂SO₄ and concentrated under reduced pressure to give a residue. The residue was purified by column chromatography (SiO₂, Petroleum ether/Ethyl acetate=3:1) to afford the title compound as a white solid (60 mg, 61.7%). ¹H NMR (400 MHz, DMSO-d₆) δ ppm 0.65-0.70 (m, 2H), 0.74-0.80 (m, 2H), 1.38 (s, 6H), 2.82-2.91 (m, 1H), 4.59 (s, 2H), 7.22 (s, 1H), 7.31-7.37 (m, 1H), 7.41-7.46 (m, 1H), 8.21 (d, J=8 Hz, 1H), 8.26 (s, 1H), 8.56 (s, 1H), 9.90 (s, 1H), F NMR (377 MHz, DMSO-d₆) δ ppm −60.02; LCMS (ESI) m/z 416.2 [M+H]⁺.

### Example 201

### 1-(4-((4-(cyclopropylamino)-5-(trifluoromethyl)pyrimidin-2-yl)amino)-1H-pyrazolo[3,4-b]pyridin-1-yl)-2-methylpropan-2-ol

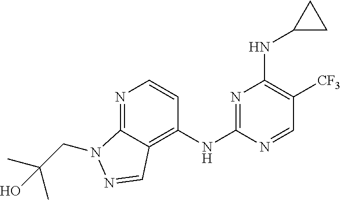 [SMILES: CC(C)(O)Cn1ncc2c(Nc3ncc(C(F)(F)F)c(NC4CC4)n3)ccnc21]

### Step 1: 1-(4-bromo-1H-pyrazolo[3,4-b]pyridin-1-yl)-2-methylpropan-2-ol

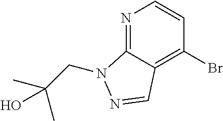

To a solution of 4-bromo-1H-pyrazolo[3,4-b]pyridine (100 mg, 0.50 mmol) and 2,2-dimethyloxirane (72.8 mg, 1.01 mmol) in DMF (2 mL) was added Cs₂CO₃ (329 mg, 1.01 mmol).

The mixture was stirred at 80° C. for 4 hrs. and then filtered. The filtrate was concentrated under reduced pressure to give a residue, which was diluted with H₂O (10 mL) and extracted with EtOAc (10 mL×3). The combined organic layers were washed with brine (20 mL), dried with anhydrous Na₂SO₄, filtered and concentrated under reduced pressure to give a residue. The residue was purified by flash silica gel chromatography (ISCO®; 4 g SepaFlash® Silica Flash Column, petroleum ether/EtOAc with EtOAc from 0-80%, 40 mL/min, 254 mu) to give 1-(4-bromopyrazolo[3,4-b]pyridin-1-yl)-2-methyl-propan-2-ol (Intermediate 201-a) as a yellow solid (180 mg, 43.5% yield). LCMS (ESI) m/z 270.0, 272.0 [M+H]⁺.

### Step 2: 1-(4-((diphenylmethylene)amino)-1H-pyrazolo[3,4-b]pyridin-1-yl)-2-methylpropan-2-ol

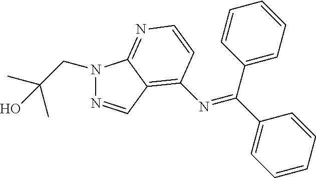 [SMILES: CC(C)(O)Cn1ncc2c(N=C(c3ccccc3)c3ccccc3)ccnc21]

A mixture of 1-(4-bromopyrazolo[3,4-b]pyridin-1-yl)-2-methyl-propan-2-ol (Intermediate 201-a) (90 mg, 0.33 mmol), diphenylmethanimine (72.5 mg, 0.40 mmol), Cs₂CO₃ (260 mg, 0.80 mmol) and Xantphos Pd G4 (32.0 mg, 0.03 mmol) in dioxane (1 mL) was degassed and purged with N₂ for 3 times, and then stirred at 90° C. for 2 hrs under N₂ atmosphere. The reaction mixture was filtered and the filtrate was concentrated under reduced pressure to give a residue, which was diluted with H₂O (10 mL) and extracted with EtOAc (10 mL×3). The combined organic layers were washed with brine (20 mL), dried with anhydrous Na₂SO₄, filtered and concentrated under reduced pressure to give a crude product 1-(4-((diphenylmethylene)amino)-1H-pyrazolo[3,4-b]pyridin-1-yl)-2-methylpropan-2-ol (Intermediate 201-b) as a brown solid (240 mg, crude), which was used into next step without purification.

### Step 3: 1-(4-amino-1H-pyrazolo[3,4-b]pyridin-1-yl)-2-methylpropan-2-ol

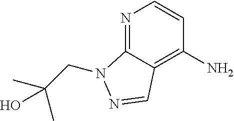

A solution of 1-[4-(benzhydrylideneamino)pyrazolo[3,4-b]pyridin-1-yl]-2-methyl-propan-2-ol (Intermediate 201-b) (240 mg, 0.65 mmol) in 2 M HCl/dioxane (2 ml) was stirred at 25° C. for 30 min. The residue was diluted with H₂O (20 mL), neutralized to pH=7˜8 by aq. NaHCO₃, and then extracted with EtOAc (10 mL×3). The combined organic layers were washed with brine (10 mL), dried with anhydrous Na₂SO₄, filtered and concentrated under reduced pressure to give a residue, which was purified by flash silica gel chromatography (ISCO®; 4 g SepaFlash® Silica Flash Column, petroleum ether/EtOAc with EtOAc from 0˜80%, 50 mL/min, 254 nm) to give 1-(4-amino-1H-pyrazolo[3,4-b]pyridin-1-yl)-2-methylpropan-2-ol (Intermediate 201-c) as a yellow oil (100 mg, 91.0%). LCMS (ESI) m/z 207.1 [M+H]⁺.

### Step 4: 1-(4-((4-(cyclopropylamino)-5-(trifluoromethyl)pyrimidin-2-yl)amino)-1H-pyrazolo[3,4-b]pyridin-1-yl)-2-methylpropan-2-ol

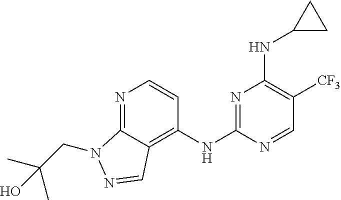 [SMILES: CC(C)(O)Cn1ncc2c(Nc3ncc(C(F)(F)F)c(NC4CC4)n3)ccnc21]

A mixture of 1-(4-aminopyrazolo[3,4-b]pyridin-1-yl)-2-methyl-propan-2-ol (Intermediate 201-c) (100 mg, 0.48 mmol), 2-chloro-N-cyclopropyl-5-(trifluoromethyl)pyrimidin-4-amine (Intermediate 68-a) (115 mg, 0.48 mmol), palladium tritert-butylphosphane (49.5 mg, 0.10 mmol) and Cs₂CO₃ (316 mg, 0.97 mmol) in DMF (2 mL) were taken up into a microwave tube. The sealed tube was heated at 120° C. for 1 hr under microwave. The reaction mixture was diluted with H₂O (10 mL) and extracted with EtOAc (10 mL×3). The combined organic layers were washed with brine (10 rnL), dried with anhydrous Na₂SO₄, filtered and concentrated under reduced pressure to give a residue. The residue was purified by prep-HPLC (column: Welch Xtimate C18 100×40 mm×3 um; mobile phase: [water (FA)-ACN]; B %: 31%-61%, 10 min) to give the title compound as a white solid (19.9 mg, 96.4%). ¹H NMR (400 MHz, DMSO-d₆) δ ppm 0.69-0.76 (m, 2H), 0.87 (d, J=8.0 Hz, 2H), 1.10 (s, 6H), 2.92 (tq, J=8.4 Hz, 1H), 4.35 (s, 2H), 4.52-5.00 (m, 1H), 7.48 (s, 1H), 8.34-8.39 (m, 2H), 8.45 (d, J=4 Hz, 1H), 8.63 (s, 1H), 10.52 (s, 1H); ¹⁹F NMR (377 MHz, DMSO-d₆) δ ppm −60.56; LCMS (ESI) m/z 408.2 [M+H]⁺.

### Example 202

### 2-methyl-2-[4-[[6-(methylamino)-9H-purin-2-yl]amino]indazol-1-yl]propanenitrile

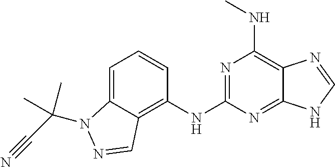 [SMILES: CNc1nc(Nc2cccc3c2cnn3C(C)(C)C#N)nc2[nH]cnc12]

### Step 1: 2-chloro-N-methyl-9H-purin-6-amine

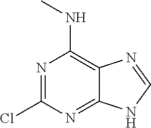

To a solution of 2,6-dichloro-9H-purine (5 g, 26.0 mmol) in EtOH (5 mL) was added MeNH₂ (3.35 g, 29.0 mmol, HCl salt). The mixture was stirred at 80° C. for 16 hrs, and then filtered. The filtrate was concentrated under reduced pressure to give a residue, which was triturated with MeOH at 25° C. to afford 2-chloro-N-methyl-9H-purin-6-amine (Intermediate 202-a) as a white solid (2.93 g, 60.4%). LCMS (ESI) m/184.1 [M+H]⁺.

### Step 2: 2-chloro-N-methyl-9-(2-trimethylsilylethoxymethyl)purin-6-amine

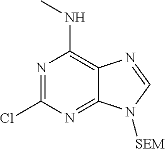 [SMILES: CNc1nc(Cl)nc2c1ncn2COCC[Si](C)(C)C]

To a solution of 2-chloro-N-methyl-9H-purin-6-amine (Intermediate 202-a) (410 mg, 2.2 mmol) in MeCN (4 mL) was added SEM-Cl (447 mg, 2.68 nmol) and Cs₂CO₃(1.46 g, 4.50 mmol). The mixture was stirred at 25° C. for 16 hrs, then filtered. The filtrate was concentrated under reduced pressure to give a residue, which was purified by column chromatography (ISCO®; 4 g SepaFlash® Silica Flash Column, petroleum ether/EtOAc with EtOAc from 0-25%, 50 mL/min, 254 nm) to afford 2-chloro-N-methyl-9-(2-trimethylsilylethoxymethyl)purin-6-amine (Intermediate 202-b) as a white solid (440 mg, 47.1%); LCMS (ESI) m/z 314.1 [M+H]⁺.

### Step 3: 2-methyl-2-[4-[[6-(methylamino)-9-(2-trimethylsilylethoxymethyl)purin-2-yl]amino]indazol-1-yl]propanenitrile

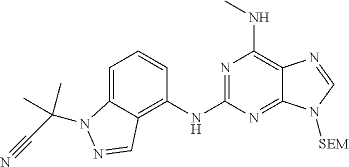 [SMILES: CNc1nc(Nc2cccc3c2cnn3C(C)(C)C#N)nc2c1ncn2COCC[Si](C)(C)C]

A mixture of 2-chloro-N-methyl-9-(2-trimethylsilylethoxymethyl)purin-6-amine (intermediate 202-b) (100 mg, 0.32 mmol), 2-(4-aminoindazol-1-yl)-2-methylpropanenitrile (Intermediate 31-d) (63.8 mg, 0.32 mmol, Pd₂(dba)₃ (58.4 mg, 63.0 μmol), SPhos (52.3 mg, 0.13 mmol) and NaOtBu (45.9 mg, 0.48 mmol) in dioxane (2 mL) was degassed and purged with N₂ for 3 times, and then stirred at 100° C. for 1 hr under N₂ atmosphere. The reaction mixture was filtered and concentrated under reduced pressure to give a residue. The residue was purified by column chromatography (ISCO®; 4 g SepaFlash® Silica Flash Column, petroleum ether/EtOAc with EtOAc from 0˜60%, 50 mL/min, 254 nm) to afford 2-methyl-2-[4-[[6-(methylamino)-9-(2-trimethylsilylethoxymethyl)purin-2-yl]amino]indazol-1-yl]propanenitrile (Intermediate 202-c) as a white solid (84 mg, 54.5%). LCMS (ESI) m/z 478.3 [M+H]⁺.

### Step 4: 2-methyl-2-[4-[[6-(methylamino)-9H-purin-2-yl]amino]indazol-1-yl]propanenitrile

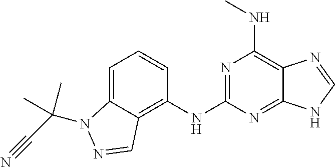 [SMILES: CNc1nc(Nc2cccc3c2cnn3C(C)(C)C#N)nc2[nH]cnc12]

To a solution of 2-methyl-2-[4-[[6-(methylamino)-9-(2-trimethylsilylethoxymethyl)purin-2-yl]amino]indazol-1-yl]propanenitrile (Intermediate 202-c) (84 mg, 0.18 mmol) in 2M HCl in EtOAc (2 mL) was added Et₃SiH (0.2 mg, 1.76 μmol). The mixture was stirred at 25° C. for 16 hrs, then diluted with NaHCO₃ aqueous solution (4 mL) and extracted with EtOAc (20 mL×3). The combined organic layers were dried over Na₂SO₄ and concentrated under reduced pressure. The crude product was triturated with MeCN at 25° C. to afford the title compound as a white solid (37.0 mg, 60.6%). ¹H NMR (400 MHz, DMSO-d₆) δ ppm 2.08 (s, 6H), 3.03 (s, 3H), 7.30-7.35 (m, 1H), 7.36-7.42 (m, 1H), 7.48 (br s, 1H), 7.83 (s, 1H), 8.22 (d, J=8 Hz, 1H), 8.62 (s, 1H), 9.09 (s, 1H), 12.48 (br s, 1H); LCMS (ESI) m/z 348.2 [M+H]⁺.

### Example 203

### 5-chloro-N⁴-cyclopropyl-N²-(1-(2-(methylsulfonyl)ethyl)-1H-indazol-4-yl)-7H-pyrrolo[2,3-d]pyrimidine-2,4-diamine

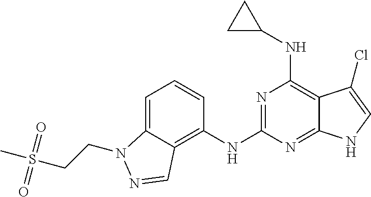 [SMILES: CS(=O)(=O)CCn1ncc2c(Nc3nc(NC4CC4)c4c(Cl)c[nH]c4n3)cccc21]

### Step 1: 2,5-dichloro-N-cyclopropyl-7H-pyrrolo[2,3-d]pyrimidin-4-amine

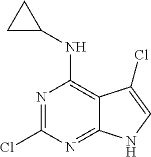 [SMILES: Clc1nc(NC2CC2)c2c(Cl)c[nH]c2n1]

To a solution of 2,4,5-trichloro-7H-pyrrolo[2,3-d]pyrimidine (1 g, 4.50 mmol) in MeCN (10 mL) was added K₂CO₃ (1.24 g, 8.99 mmol) and cyclopropanamine (385 mg, 6.74 mmol). The mixture was stirred at 25° C. for 16 hrs, then diluted with H₂O (10 mL) and extracted with EtOAc (50 mL×3). The combined organic layers were washed with brine (20 mL), dried with anhydrous Na₂SO₄, filtered and concentrated under reduced pressure to give a residue, which was purified by flash chromatography (ISCO®; 4 g SepaFlash® Silica Flash Column, petroleum ether/EtOAc with EtOAc from 0-30%, 40 mL/min, 254 nm) to provide 2,5-dichloro-N-cyclopropyl-7H-pyrrolo[2,3-d]pyrimidin-4-amine (Intermediate 203-a) as a white solid (460 mg, 39.9%). LCMS (ESI) m/z 242.9, 244.9 [M+H]⁺.

### Step 2: 2,5-dichloro-N-cyclopropyl-7-((2-(trimethylsilyl)ethoxy)methyl)-7H-pyrrolo[2,3-d]pyrimidin-4-amine

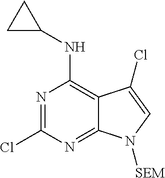 [SMILES: C[Si](C)(C)CCOCn1cc(Cl)c2c(NC3CC3)nc(Cl)nc21]

To a solution of 2,5-dichloro-N-cyclopropyl-7H-pyrrolo[2,3-d]pyrimidin-4-amine (Intermediate 203-a) (400 mg, 1.65 mmol) in THF (5 mL) was added Cs₂CO₃ (1.07 g, 3.29 mmol) and SEM-Cl (329 mg, 1.97 mmol). The mixture was stirred at 25° C. for 16 hrs, then diluted with H₂O (10 mL) and extracted with EtOAc (50 mL×3). The combined organic layers were washed with brine (20 mL), dried with anhydrous Na₂SO₄, filtered and concentrated under reduced pressure to give a residue, which was purified by flash chromatography (ISCO®; 4 g SepaFlash® Silica Flash Column, petroleum ether/EtOAc with EtOAc from 0˜30%, 40 mL/min, 254 nm) to afford 2,5-dichloro-N-cyclopropyl-7-(2-trimethylsilylethoxymethyl)pyrrolo[2,3-d]pyrimidin-4-amine (intermediate 203-b) as a colorless oil (210 mg, 33.8%). LCMS (ESI) m/z 373.1, 375.1 [M+H]⁺.

### Step 3: 5-chloro-N⁴-cyclopropyl-N²-(1-(2-(methylsulfonyl)ethyl)-1H-indazol-4-yl)-7-((2-(trimethylsilyl)ethoxy)methyl)-7H-pyrrolo[2,3-d]pyrimidine-2,4-diamine

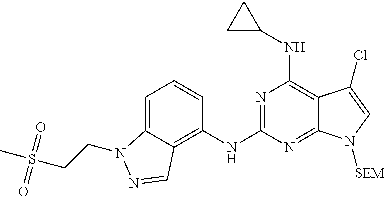 [SMILES: C[Si](C)(C)CCOCn1cc(Cl)c2c(NC3CC3)nc(Nc3cccc4c3cnn4CCS(C)(=O)=O)nc21]

A mixture of 2,5-dichloro-N-cyclopropyl-7-(2-trimethylsilylethoxymethyl)pyrrolo[2,3-d]pyrimidin-4-amine (Intermediate 203-b) (120 mg, 0.32 mmol, 1 eq), 1-(2-methylsulfonylethyl)indazol-4-amine (Intermediate 139-b) (76.9 mg, 0.32 mmol), Cs₂CO₃ (314 mg, 0.96 mmol) and Pd(t-Bu₃P) (33.0 mg, 64.3 μmol) in DMF (2 mL) was degassed and purged with N₂ for 3 times, and then stirred at 100° C. for 1 hr under microwave. The reaction mixture was diluted by H₂O (10 mL) and extracted with EtOAc (50 ml×3). The combined organic layers were washed with brine (20 mL), dried with anhydrous Na₂SO₄, filtered and concentrated under reduced pressure to give a residue, which was purified by flash chromatography (ISCO®; 4 g SepaFlash® Silica. Flash Column, petroleum ether/EtOAc with EtOAc from 0˜60%, 40 ml/min, 254 nm) to afford 5-chloro-N⁴-cyclopropyl-N²-[1-(2-methylsulfonylethyl)indazol-4-yl]-7-(2-trimethylsilylethoxymethyl)pyrrolo[2,3-d]pyrimidine-2,4-diamine (intermediate 203-c) as a yellow solid (70 mg, 27.9%). LCMS (ESI) m/z 576.3 [M+H]⁺.

### Step 4: (5-chloro-4-(cyclopropylamino)-2-((1-(2-(methylsulfonyl)ethyl)-1H-indazol-4-yl)amino)-7H-pyrrolo[2,3-d]pyrimidin-7-yl)methanol

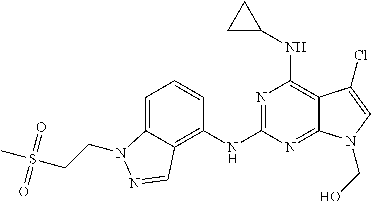 [SMILES: CS(=O)(=O)CCn1ncc2c(Nc3nc(NC4CC4)c4c(Cl)cn(CO)c4n3)cccc21]

To a solution of 5-chloro-N⁴-cyclopropyl-N²-[1-(2-methylsulfonylethyl)indazol-4-yl]-7-(2-trimethylsilylethoxymethyl)pyrrolo[2,3-d]pyrimidine-2,4-diamine (Intermediate 203-c) (170 mg, 0.17 mmol) in DCM (2 rnL) was added TFA (1.45 g, 12.8 mmol). The mixture was stirred at 25° C. for 2 hrs, then concentrated under reduced pressure to give a crude product [5-chloro-4-(cyclopropylamino)-2-[[1-(2-methylsulfonylethyl)indazol-4-yl]amino]pyrrolo[2,3-d]pyrimidin-7-yl]methanol (Intermediate 203-d) as a yellow oil (84 mg, 99.7%). LCMS (ESI) m/z 476.2 [M+H]⁺.

### Step 5: 5-chloro-N4-cyclopropyl-N2-(1-(2-(methylsulfonyl)ethyl)-1H-indazol-4-yl)-7H-pyrrolo[2,3-d]pyrimidine-2,4-diamine

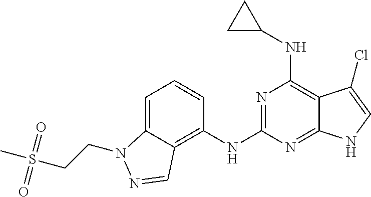 [SMILES: CS(=O)(=O)CCn1ncc2c(Nc3nc(NC4CC4)c4c(Cl)c[nH]c4n3)cccc21]

To a solution of [5-chloro-4-(cyclopropylamino)-2-[[1-(2-methylsulfonylethyl)indazol-4-yl]amino]pyrrolo[2,3-d]pyrimidin-7-yl]methanol (Intermediate 203-d) (84 mg, 0.17 mmol) in THF (2 ml) and H₂O (0.4 mL) was added LiOH (42.3 mg, 1.76 mmol). The mixture was stirred at 25° C. for 2 hrs, diluted with H₂O (10 mL) and extracted with EtOAc (50 mL×3). The combined organic layers were washed with brine (20 mL), dried with anhydrous Na₂SO₄, filtered and concentrated under reduced pressure. The residue was purified by prep-HPLC (column: Phenomenex Synergi C18 100×30 mm×4 um; mobile phase: [water (HCl)-ACN]; B %: 20%-50%, 8 min) to provide the title compound as a white solid (17.5 mg, 19.5% yield) ¹H NMR (400 MHz, CD₃OD) δ ppm 0.95 (br s, 2H), 1.15 (br d, J=3.88 Hz, 2H), 2.79 (s, 3H), 2.88-2.98 (m, 1H), 3.79 (t, J=6.50 Hz, 2H), 4.90 (t, J=6.50 Hz, 2H), 7.09 (s, 1H), 7.42-7.51 (m, 2H), 8.05 (br d, J=6.50 Hz, 1H), 8.35 (s, 1H); LCMS (ESI) m/z 446.2 [M+H]⁺.

### Example 204

### 1-[4-[[4-(cyclopropylamino)-3-methyl-1H-pyrazolo[3,4-d]pyrimidin-6-yl]amino]indazol-1-yl]-2-methyl-propan-2-ol

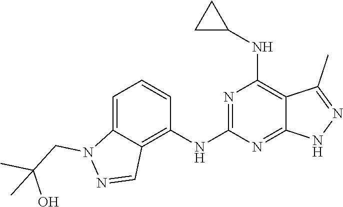

### Step 1: 2-[(4,6-dichloro-3-methyl-pyrazolo[3,4-d]pyrimidin-1-yl)methoxy]ethyl-trimethyl-silane

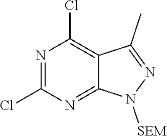 [SMILES: Cc1nn(COCC[Si](C)(C)C)c2nc(Cl)nc(Cl)c12]

To a solution of 4,6-dichloro-3-methyl-1H-pyrazolo[3,4-d]pyrimidine (1.7 g, 8.37 mmol) in MeCN (1 mL) was added SEM-Cl (1.40 g, 8.37 mmol) and Cs₂CO₃ (8.18 g, 25.1 mmol). The mixture was stirred at 25° C. for 16 hrs, diluted with H₂O (30 mL) and extracted with EtOAc (90 mL). The combined organic layers were washed with brine (60 mL), dried with anhydrous Na₂SO₄, filtered and concentrated under reduced pressure. The residue was purified by flash silica gel chromatography (ISCO®; 4 g SepaFlash® Silica Flash Column, petroleum ether/EtOAc with EtOAc from 0˜7%, 40 mL/min, 254 nm) to give 2-[(4,6-dichloro-3-methyl-pyrazolo[3,4-d]pyrimidin-1-yl)methoxy]ethyl-trimethyl-silane (Intermediate 204-a) as a white solid (1.3 g, 46.5% yield). LCMS (ESI) m/z 333.1, 335.1 [M+H]⁺.

### Step 2: 6-chloro-N-cyclopropyl-3-methyl-1-(2-trimethylsilylethoxymethyl)pyrazolo[3,4-d]pyrimidin-4-amine

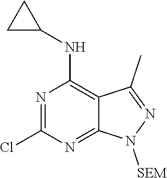 [SMILES: Cc1nn(COCC[Si](C)(C)C)c2nc(Cl)nc(NC3CC3)c12]

To a solution of 2[(4,6-dichloro-3-methyl-pyrazolo[3,4-d]pyrimidin-1-yl)methoxy]ethyl-trimethyl-silane (Intermediate 204-a) (600 mug, 1.80 mmol) in MeCN (6 mL) was added cyclopropanamine (123 mg, 2.16 mmol) and K₂CO₃ (746 mg, 5.40 mmol). The mixture was stirred at 25° C. for 16 hrs, diluted with H₂O (30 ml) and extracted with EtOAc (90 mL). The combined organic layers were washed with brine (60 mL), dried with anhydrous Na₂SO₄, filtered and concentrated under reduced pressure to give a residue. The residue was purified by flash silica gel chromatography (ISCO®; 4 g SepaFlash® Silica Flash Column, petroleum ether/EtOAc with EtOAc from 0˜20%, 40 mL/min, 254 nm) to give 6-chloro-N-cyclopropyl-3-methyl-1-(2-trimethylsilylethoxymethyl)pyrazolo[3,4-d]pyrimidin-4-amine (Intermediate 204-h) as an off-white solid (621 mg, 95.8% yield). LCMS (ESI) m/z 354.2 [M+H]⁺.

### Step 3: 1-[4-[[4-(cyclopropylamino)-3-methyl-1-(2-trimethylsilylethoxymethyl)pyrazolo[3,4-d]pyrimidin-6-yl]amino]indazol-1-yl]-2-methyl-propan-2-ol

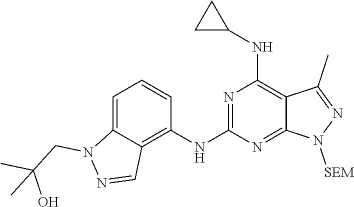

A mixture of 6-chloro-N-cyclopropyl-3-methyl-1-(2-trimethylsilylethoxymethyl)pyrazolo[3,4-d]pyrimidin-4-amine (Intermediate 204-b) (120 mg, 0.34 mmol), 1-(4-aminoindazol-1-yl)-2-methylpropan-2-ol (Intermediate 97-b) (76.5 mg, 0.37 mmol), SPhos (27.8 mg, 0.067 mmol), Pd₂(dba)₃ (31.1 mg, 0.034 mmol) and t-BuONa (81.5 mg, 0.85 mmol) in dioxane (2 mL) was degassed and purged with N₂ for 3 times and then stirred at 100° C. for 16 hrs under N₂ atmosphere. The reaction mixture was diluted with H₂O (15 mL) and extracted with EtOAc (30 mL). The combined organic layers were washed with brine (30 mL), dried with anhydrous Na₂SO₄, filtered and concentrated under reduced pressure. The residue was purified by flash silica gel chromatography (ISCO®; 12 g SepaFlash® Silica Flash Column, petroleum ether/EtOAc with EtOAc from 0˜60%, 40 mL/min, 254 nm) to give 1-[4-[[4-(cyclopropylamino)-3-methyl-1-(2-trimethylsilylethoxymethyl)pyrazolo[3,4-d]pyrimidin-6-yl]amino]indazol-1-yl]-2-methyl-propan-2-ol (Intermediate 204-c) as a yellow solid (80 mg, 45.1% yield). LCMS (ESI) m/z 523.4 [M+H]⁺.

### Step 4:1-[4-[[4-(cyclopropylamino)-3-methyl-1H-pyrazolo[3,4-d]pyrimidin-6-yl]amino]indazol-1-yl]-2-methyl-propan-2-ol

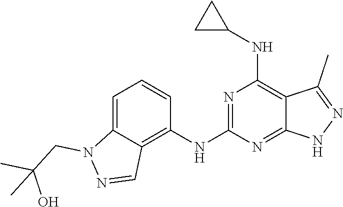

To a solution of 1-[4-[[4-(cyclopropylamino)-3-methyl-1-(2-trimethylsilylethoxymethyl)pyrazolo[3,4-d]pyrimidin-6-yl]amino]indazol-1-yl]-2-methyl-propan-2-ol (Intermediate 204-c) (80 mg, 0.15 mmol) in 2M HCl in MeOH (2 mL) was added Et₃SiH (2 mg, 0.015 nmol). The mixture was stirred at 25° C. for 1 hr and then filtered. The filtrate was concentrated under reduced pressure to give the title compound as a yellow solid (47.7 mg, 77.1% yield). ¹H NMR (400 MHz, CD₃OD) δ ppm 0.78-0.86 (m, 2H), 0.90 (br d, J=7.24 Hz, 2H), 1.24 (s, 6H), 2.65 (s, 3H), 2.96-3.06 (m, 1H), 4.39 (s, 2H), 7.41-7.49 (m, 1H), 7.51-7.59 (m, 1H), 7.73 (br d, J=7.28 Hz, 1H), 8.22 (s, 1H); LCMS (ESI) m/z 393.3 [M+H]⁺.

### Example 205

### 1-[4-[[4-(ethylamino)-3-methyl-1H-pyrazolo[3,4-d]pyrimidin-6-yl]amino]-7-fluoro-indazol-1-yl]-2-methyl-propan-2-ol

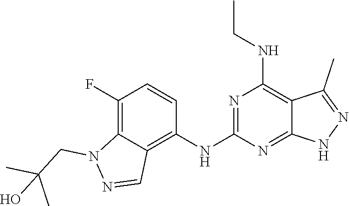

### Step 1: 6-chloro-N-ethyl-3-methyl-1-(2-trimethylsilylethoxymethyl)pyrazolo[3,4-d]pyrimidin-4-amine

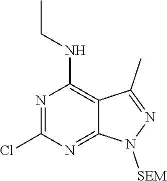 [SMILES: CCNc1nc(Cl)nc2c1c(C)nn2COCC[Si](C)(C)C]

To a solution of 2-[(4,6-dichloro-3-methyl-pyrazolo[3,4-d]pyrimidin-1-yl)methoxy]ethyl-trimethyl-silane (Intermediate 204-a) (600 mg, 1.80 mmol) in MeCN (10 mL) was added ethanamine hydrochloride (254 mg, 2.16 mmol, 1.2 eq) and K₂CO₃ (746 mg, 5.40 mmol, 3 eq). The mixture was stirred at 25° C. for 16 hr, diluted with H₂O (30 mL) and extracted with EtOAc (30 mL×3). The combined organic layers were washed with brine (30 mL×2), dried over Na₂SO₄, filtered and concentrated under reduced pressure. The residue was purified by flash chromatography (ISCO®; 40 g SepaFlash® Silica Flash Column, petroleum ether/EtOAc with EtOAc from 0˜20%, 35 mL/min, 254 nm) to give 6-chloro-N-ethyl-3-methyl-1-(2-trimethylsilylethoxymethyl)pyrazolo[3,4-d]pyrimidin-4-amine (Intermediate 205-a) as a white solid (600 mg, 92.6%). LCMS (ESI) m/z 342.2 [M+H]⁺.

### Step 2: 1-[4-[[4-(ethylamino)-3-methyl-1-(2-trimethylsilylethoxymethyl)pyrazolo[3,4-d]pyrimidin-6-yl]amino]-7-fluoro-indazol-1-yl]-2-methyl-propan-2-ol

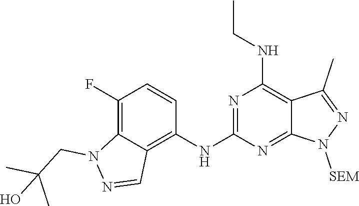

A mixture of 6-chloro-N-ethyl-3-methyl-1-(2-trimethylsilylethoxymethyl)pyrazolo[3,4-d]pyrimidin-4-amine (Intermediate 205-a) (150 mg, 0.43 mmol), 1-(4-amino-7-fluoro-indazol-1-yl)-2-methyl-propan-2-ol (Intermediate 172-c) (97.9 mig, 0.43 mmol), SPhos (72.0 mg, 0.17 mmol), Pd₂(dba)₃ (80.5 mg, 0.08 mmol) and t-BuONa (105 mg, 1.10 mmol) in dioxane (1 mL) was degassed and purged with N² for 3 times and then stirred at 100° C. for 16 hrs under N₂ atmosphere. The residue was diluted with H₂O (10 mL) and extracted with EtOAc (10 mL×3). The combined organic layers were washed with brine (10 mL×2), dried over Na₂SO₄, filtered and concentrated under reduced pressure. The residue was purified by flash chromatography (ISCO®, 12 g SepaFlash® Silica Flash Column, petroleum ether/EtOAc with EtOAc from 0˜60%, 35 mL/min, 254 nm) to give 1-[4-[[4-(ethylamino)-3-methyl-1-(2-trimethylsilylethoxymethyl)pyrazolo[3,4-d]pyrimidin-6-yl]amino]-7-fluoro-indazol-1-yl]-2-methyl-propan-2-ol (Intermediate 205-b) as a white solid (100 mg, 41.0% G) LCMS (ESI) m/z 529.2 [M+H]⁺.

### Step 3: 1-[4-[[4-(ethylamino)-3-methyl-1H-pyrazolo[3,4-d]pyrimidin-6-yl]amino]-7-fluoro-indazol-1-yl]-2-methyl-propan-2-ol

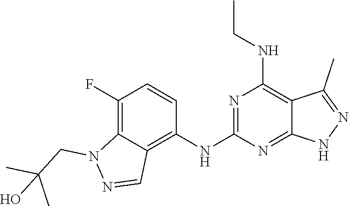

To a solution of 1-[4-[[4-(ethylamino)-3-methyl-1-(2-trimethylsilylethoxymethyl)pyrazolo[3,4-d]pyrimidin-6-yl]amino]-7-fluoro-indazol-1-yl]-2-methyl-propan-2-ol (Intermediate 205-b) (90.0 mg, 0.17 mmol) in 2M HCl in MeOH (1 mL) was added Et₃SiH (19.7 mg, 0.17 mmol). The mixture was stirred at 25° C. for 1 hr and concentrated under reduced pressure. The crude product was triturated with MeCN at 25° C. for 30 min to give the title compound as a white solid (54.37 mg, 72.6%). ¹H NMR (400 MHz, DMSO-d₆) 5 ppm 1.10 (s, 6H), 1.17 (br t, J=6.8 Hz, 3H), 2.63 (s, 3H), 3.50-3.54 (m, 2H), 4.38 (s, 2H), 7.22 (dd, J=12, 8 Hz, 1H), 7.60 (br s, 1H), 8.51 (br s, 2H), 10.78 (br s, 1H), 12.67 (br s, 1H); LCMS (ESI) m/z 399.3 [M+H]⁺.

### Example 206

The compounds in Table 1 were tested for their inhibitory effect on LRRK2 kinase according to the following procedures.

Biochemical Assays:

The protocol for basic TR-FRET LanthaScreen Tb Kinase Activity Assay inhibitor studies were as follows. LanthaScreen Kinase Activity Assays (ThermoFisher/USA) to evaluate inhibitors were performed by addition of 100 nL of test compound in corresponding DMSO dilutions/5 μL of kinase/fluorescein-ERM(LRRKtide) peptide mixture, 5 μL of ATP into 384 well small volume plates. After incubation for 120 minutes at room temperature, the detection reagents containing Tb-anti-pLRRKtide antibody were added to monitor phosphrylation level of peptide. Then, after 60 min minutes incubation at room temperature plates were read in Envision. Data analysis of emission ratios was according to LanthaScreen Tb Kinase Activity Assay protocol.

Kinase and assay components were adjusted to final concentrations according to the kit protocol. For LRRK2: 2 nM wt human LRRK2, catalytic site, catalytic site (ThermoFisher/USA), 400 nM peptide, 38 μM ATP in 1× Kinase Buffer A.

Basic protocol for TR-FRET LanthaScreen Tb Kinase Activity Assay inhibitor studies involved two steps:
Enzymatic step: Addition of 100 nL of test compound in corresponding DMSO dilutions, 5 μL of kinase/substrate mixture, 5 μL of ATP into 384 well small volume plates. Incubation for 120 minutes at room temperature.
Detection step: Addition of 10 μL EDTA & antibody, read plate after 60 minutes. Data analysis of emission ratios according to KinEASE assay protocol.

Kinase and assay components were adjusted to final concentrations according to the kit protocol.

Compounds disclosed herein on Table 1 were tested according to methods described in Example 206 and found to exhibit an LRRK2 IC₅₀ as indicated in Table 2. In the table below, activity is provided as follows: +++=IC₅₀ less than 50 nM; ++=IC₅₀ between 50 nM and 200 nM; +=IC₅₀>=200 nM

TABLE 2

| Example | LRRK2 | Example | LRRK2 | Example | LRRK2 | Example | LRRK2 |
|---|---|---|---|---|---|---|---|
| No. | IC₅₀ | No. | IC₅₀ | No. | IC₅₀ | No. | IC₅₀ |
| 1 | +++ | 2 | +++ | 3 | +++ | 4 | ++ |
| 5 | +++ | 6 | +++ | 7 | +++ | 8 | + |
| 9 | +++ | 10 | +++ | 11 | +++ | 12 | +++ |
| 13 | +++ | 14 | ++ | 15 | ++ | 16 | +++ |
| 17 | +++ | 18 | ++ | 19 | +++ | 20 | +++ |
| 21 | +++ | 22 | ++ | 23 | ++ | 24 | +++ |
| 25 | +++ | 26 | +++ | 27 | +++ | 28 | +++ |
| 29 | +++ | 30 | +++ | 31 | +++ | 32 | +++ |
| 33 | +++ | 34 | +++ | 35 | +++ | 36 | ++ |
| 37 | +++ | 38 | ++ | 39 | + | 40 | + |
| 41 | + | 42 | +++ | 43 | +++ | 44 | +++ |
| 45 | +++ | 46 | +++ | 47 | +++ | 48 | ++ |
| 49 | +++ | 50 | +++ | 51 | +++ | 52 | +++ |
| 53 | ++ | 54 | ++ | 55 | +++ | 56 | +++ |
| 57 | +++ | 58 | +++ | 59 | +++ | 60 | +++ |
| 61 | +++ | 62 | ++ | 63 | ++ | 64 | ++ |
| 65 | + | 66 | ++ | 67 | + | 68 | +++ |
| 69 | +++ | 70 | +++ | 71 | +++ | 72 | +++ |
| 73 | +++ | 74 | +++ | 75 | ++ | 76 | + |
| 77 | +++ | 78 | +++ | 79 | +++ | 80 | +++ |
| 81 | +++ | 82 | +++ | 83 | +++ | 84 | +++ |
| 85 | +++ | 86 | +++ | 87 | +++ | 88 | +++ |
| 89 | +++ | 90 | +++ | 91 | +++ | 92 | +++ |
| 93 | +++ | 94 | +++ | 95 | +++ | 96 | +++ |
| 97 | +++ | 98 | +++ | 99 | +++ | 100 | +++ |
| 101 | +++ | 102 | +++ | 103 | +++ | 104 | +++ |
| 105 | +++ | 106 | +++ | 107 | +++ | 108 | +++ |
| 109 | +++ | 110 | + | 111 | +++ | 112 | +++ |
| 113 | +++ | 114 | +++ | 115 | + | 116 | +++ |
| 117 | + | 118 | +++ | 119 | +++ | 120 | ++ |
| 121 | + | 122 | ++ | 123 | + | 124 | + |
| 125 | +++ | 126 | + | 127 | +++ | 128 | ++ |
| 129 | +++ | 130 | +++ | 131 | +++ | 132 | ++ |
| 133 | +++ | 134 | +++ | 135 | +++ | 136 | +++ |
| 137 | +++ | 138 | +++ | 139 | +++ | 140 | +++ |
| 141 | +++ | 142 | +++ | 143 | +++ | 144 | +++ |
| 145 | +++ | 146 | +++ | 147 | +++ | 148 | +++ |
| 149 | +++ | 150 | +++ | 151 | +++ | 152 | +++ |
| 153 | +++ | 154 | +++ | 155 | +++ | 156 | +++ |
| 157 | +++ | 158 | +++ | 159 | +++ | 160 | +++ |
| 161 | +++ | 162 | +++ | 163 | +++ | 164 | +++ |
| 165 | +++ | 166 | +++ | 167 | +++ | 168 | +++ |
| 169 | +++ | 170 | +++ | 171 | +++ | 172 | +++ |
| 173 | +++ | 174 | +++ | 175 | +++ | 176 | +++ |
| 177 | +++ | 178 | +++ | 179 | +++ | 180 | +++ |
| 181 | +++ | 182 | +++ | 183 | +++ | 184 | +++ |
| 185 | +++ | 186 | +++ | 187 | +++ | 188 | +++ |
| 189 | +++ | 190 | +++ | 191 | +++ | 192 | +++ |
| 193 | +++ | 194 | +++ | 195 | +++ | 196 | +++ |
| 197 | ++ | 198 | +++ | 199 | +++ | 200 | +++ |
| 201 | +++ | 202 | +++ | 203 | +++ | 204 | +++ |
| 205 | +++ | | | | | | |

### INCORPORATION BY REFERENCE

References arnd citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A compound of Formula (I): 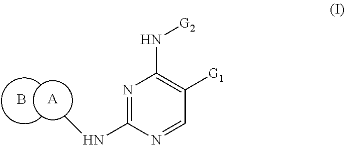
and pharmaceutically acceptable salts thereof,
wherein:
G₁ is CF₃, CHF₂, CH₂F, halogen or cyclopropyl, ethyl, isopropyl;
G₂ is H, substituted or unsubstituted C₁-C₆-alkyl, C₃-C₆-cycloalkyl, C₁-C₆ haloalkyl, C₃-C₆-halocycloalkyl, C₃-C₆-heterocycloalkyl, heteroaryl, —CH₂-cycloalkyl, —CF₂-cycloalkyl, —CH(CH₃)-cycloalkyl, —CH₂-aryl, —CH₂-heterocycloalkyl, —CH₂-heteroaryl, —CF₂-aryl, and —CH(—CH₃)-aryl;
A and B are independently selected from 5 or 6 membered cycloalkyl or aryl rings comprising one or more heteroatoms and one or more substitutions;
wherein both A and B comprise, cumulatively, at least two (2) nitrogen heteroatoms;
wherein A and B are fused at 2 positions;
wherein the one or more substitutions on the A and B rings are independently selected from: H, halo, C₁-C₆-alkyl, branched alkyl, haloalkyl, substituted or unsubstituted alkenyl, substituted or unsubstituted alkynyl, alkoxy, cycloalkoxy, haloalkoxy, alkoxyalkoxy, cyano, —CH₂-cycloalkyl, —CF₂-cycloalky, —(CH₂)₀₋₂C(CH₃)₂—CN, —(CH₂)₀₋₄—CN, —(CH₂)₀₋₄SO₂R′, —CH(CH₃)-cycloalkyl, —CH₂-aryl, —CF₂-aryl, —CH(—CH₃)-aryl, —C(═O)-alkyl, —C(═O)cycloalkyl, —C(═O)—NR′R″, —C(═O)—NH-alkyl, —C(═O)NH₂, hydroxy, —COOH (and ester thereof), alkylsulfonyl, cycloalkylsulfonyl, arylsulfonyl, —S(═O)₂—NR′R″, —NH₂, —NR′R″, —NR′—S(═O)₂R″, cyanoalkyl, haloalkyl, substituted or unsubstituted alkylsulfonyl, arylsulfonyl, —C(═O)-morpholine, —C(═O)-heterocycles, —C(H)₀₋₁(—CH₃)₁₋₂—OH, —CH₂—C(═O)—NH₂; 3-6 membered heterocycle, any of which may have one or more substituents, wherein the 3-6 membered heterocycle comprises at least one heteroatom independently selected from O, S, and N;
wherein R′ and R″ are independently selected from a group consisting of H, alkyl, substituted or unsubstituted aryl, and heterocycles.

2. The compound of claim 1 or a pharmaceutically acceptable salt thereof, the compound of the invention is a compound of Formula (II): 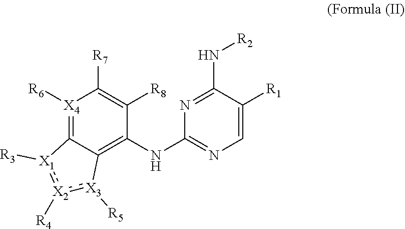
wherein,
X₁, X₂, X₃, and X₄ are C or N, wherein X₁, X₂, and X₃ include either one (1) N, two (2) N, or three (3) N;
R₁ is CF₃, CHF₂, CH₂F, halogen, cyclopropyl, ethyl, isopropyl;
R₂ is H, substituted or unsubstituted C₁-C₆ alkyl, C₃-C₆-cycloalkyl, C₃-C₆-halocycloalkyl; branched alkyl, alkenyl, alkynyl, haloalkyl, alkoxy, cycloalkoxy, haloalkoxy, —CH₂-cycloalkyl, —CF₂-cycloalky, tetrahydrofuran, —CH(—CH₃)-cycloalkyl, —CH₂-aryl, —CF₂-aryl, —CH(—CH₃)-aryl, —C(═O)-alkyl, —C(═O)cycloalkyl, —C(═O)—NH-alkyl;
R₃, R₄, R₅ are independently selected from a group consisting of: H, substituted or unsubstituted C₁-C₆ alkyl, C₃-C₆-cycloalkyl, C₃-C₆-halocycloalkyl; branched alkyl, alkenyl, alkynyl, haloalkyl, alkoxy, cycloalkoxy, haloalkoxy, nitro, cyano, alkylcyano, —CH₂-cycloalkyl, —CF₂-cycloalky, —CH(CH₃)-cycloalkyl, —CH₂-aryl, —CF₂-aryl, —CH(—CH₃)-aryl, —C(═O)—R′, —C(═O)—NR′R″, —S═(O)₂—R′, —CH₂CN, —C—(CH₃)₂—CN, —C(—CH₃)₂—OH, —CH₂—C(═O)—NH₂;
R₆, R₇, and R₈ are independently selected from a group consisting of: H, halo, C₁-C₆-alkyl, branched alkyl, alkenyl, alkynyl, haloalkyl, alkoxy, cycloalkoxy, haloalkoxy, nitro, cyano, —CH₂-cycloalkyl, —CF₂-cycloalky, —CH(CH₃)-cycloalkyl, —CH₂-aryl, —CF₂-aryl, —CH(—CH₃)-aryl, —C(═O)-alkyl, —C(═O)cycloalkyl, —C(═O)—NH-alkyl, —C(═O)NH₂, hydroxy, —COOH (and ester thereof), —C(═O)-morpholine, —C(═O)-heterocycle, —S═(O)₂—R′, amino, —NR′R″, —NR'S═(O)₂—R′, cyanoalkyl, haloalkyl, —C(—CH₃)₂—OH, —CH₂—C(═O)—NH₂; 3-6 membered heterocycle, any of which may have one or more substituents, wherein the 3-6 membered heterocycle comprises at least one heteroatom independently selected from O, S, and N; —S═(O)₂-cyclopropyl, —C(═O)-morpholine, —C(—CH₃)₂—OH, —CH₂—C(═O)—NH₂, —CF₃, —OCF₃, tetrahydropyran, 3H-pyran, 2H-pyran, piperidine, alkyl-morpholine;
wherein R′ and R″ are independently selected from a group consisting of H, unsubstituted or substituted C₁-C₈ alkyl, substituted or unsubstituted C₁-C₈ aryl, substituted or unsubstituted C₁-C₈ cycloalkyl or heterocycloalkyl;
wherein substitutions R₃, R₄, R₅, and R₆ are only present where valency permits; and
wherein R₃ and R₆ optionally form a 5-7 membered heteroalkyl ring.

3. The compound of claim 2, wherein R₁ is CF₃.

4. The compound of claim 2, wherein R₁ is CHF₂.

5. The compound of claim 2, wherein R₁ is chloro.

6. The compound of claim 2, wherein R₂ is selected from the group consisting of: H, methyl, ethyl, cyclopropyl, halogentated cyclopropyl, ethyl, cyclopentyl, tetrahydrofuran, cyclobutyl, hydroxycyclobutyl, halocyclopropyl, fluorocycopropyl, diflurocyclobutyl, isopropyl, —CH₂CF₃, —CH₂—CH₂—F, —CH₂—CH₂—OH, —CH₂—CH₂—O—CH₃, —CH₂—CH₂S═(O)₂—CH₃, —CH₂-cyclopropyl, —N(CH₃)₂, —CH(CH₃)₂, cyclobutyl, and —CH(CH₃)cyclopropyl.

7. The compound of claim 2, wherein R₃, R₄, R₅ are independently selected from a group consisting of: H, —CH₃, —CH₂—CN, —CH₂—CH₂—O—CH₃, —CH₂—CHF₂, isopropyl, cyanoisopropyl, cyclopropyl, t-butyl, —CH₂-cyclopropyl, —CH₂—C(CH₃)₂—CN, —S═(O)₂—CH₃, —CH₂—CH₂S═(O)₂—CH₃, —C—(CH₃)₂—CN, —S═(O)₂—CH—(CH₃)₂, —S═(O)₂-cyclopropyl, —CH₂—CH₂—S═(O)₂—CH₂—CH₃, —C—(CH₃)₂—S═(O)₂—CH—(CH₃)₂, —S═(O)₂—CH₂—CH₂—OCH₃, -morpholine, —CH₂-oxetane, C(═O)-morpholine, —C(—CH₃)₂—OH, —CH₂—C(═O)—NH₂.

8. The compound of claim 2, wherein R₆, R₇, and R₈ independently selected from the group consisting of: H, fluoro, methyl, chloro, cyano, —O—CH₃, CH₃, —CH—(CH₃)₂, —CH₂—CN, —CH₂—CHF₂, —S═(O)₂—CH₃, —C—(CH₃)₂—CN, —S═(O)₂-cyclopropyl, —C(═O)-morpholine, —C(—CH₃)₂—OH, —CH₂—C(═O)—NH₂, —CF₃, —OCF₃, tetrahydropyran, 3H-pyran, 2H-pyran, piperidine, methylpiperidine, and alkyl-morpholine.

9. The compound of claim 2, wherein X₁ and X₂ are N, and X₃ is C.

10. The compound of claim 2, wherein R₁ and R₂ together form a ring.

11. The compound of claim 2, wherein R₃ and R₆ together form a fused ring.

12. The compound of claim 2, wherein R₃ and R₆ together form a morpholine ring.

13. The compound of claim 1 or a pharmaceutically acceptable salt thereof, wherein the compound is a compound of Formula (III): 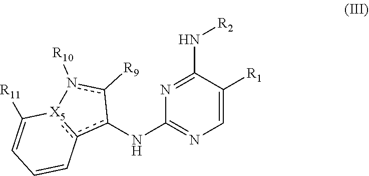
wherein, X is selected from C or N;
R₉, R₁₀, or R₁₁ are independently selected from a group consisting of: H, substituted or unsubstituted C₁-C₆ alkyl, C₁-C₆-cycloalkyl, C₃-C₆-halocycloalkyl; C₁-C₆-alkyl, branched alkyl, alkenyl, alkynyl, haloalkyl, alkoxy, cycloalkoxy, haloalkoxy, nitro, cyano, —CH₂-cycloalkyl, —CF₂-cycloalky, —CH(CH₃)-cycloalkyl, —CH₂-aryl, —CF₂-aryl, —CH(—CH₃)-aryl, C(═O)-alkyl, —C(═O)cycloalkyl, —C(═O)—NH-alkyl, —S═(O)₂—CH₃, —C—(CH₃)₂—CN, —S═(O)₂-cyclopropyl, —C(═O)-morpholine, —C(—CH₃)₂—OH, —CH₂—C(═O)—NH₂;
wherein substitutions R₉, R₁₀, and R₁₁ are only present where valency permits.

14. The compound of claim 13, wherein X₅ is C.

15. The compound of claim 13, wherein X₅ is N.

16. The compound of claim 13 wherein R₉, R₁₀, or R₁₁ are independently selected from a group consisting of: H, fluoro, chloro, cyano, CH₃, —O—CH₃, —CH—(CH₃)₂, or —CH₂—CN.

17. The compound of claim 1 or a pharmaceutically acceptable salt thereof, wherein the compound is a compound of Formula (IV): 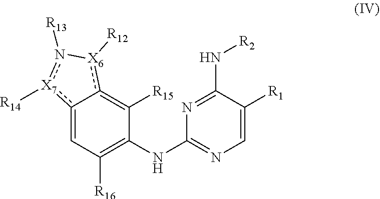
wherein, X₆ and X₇ are selected from C or N, wherein at least one of X₆ and X₇ is N;
R₁₂, R₁₃, and R₁₄ are independently selected from a group consisting of: H, substituted or unsubstituted C₁-C₆ alkyl, C₃-C₆-cycloalkyl, C₃-C₆-halocycloalkyl; C₁-C₆ branched alkyl, C₁-C₆-alkenyl, C₁-C₆-alkynyl, C₁-C₆-haloalkyl, C₁-C₆-alkoxy, cycloalkoxy, haloalkoxy, halo, nitro, cyano, —CH₂-cycloalkyl, —CF₂-cycloalky, —CH(CH₃)-cycloalkyl, —CH₂-aryl, —CF₂-aryl, —CH(—CH₃)-aryl, C(═O)-alkyl, —C(═O)heterocycloalkyl, and —C(═O)-morpholine;
R₁₅ and R₁₆ are independently selected from a group consisting of: H, halo, substituted or unsubstituted C₁-C₆ alkyl, alkyloxy, cyanoalkyl, C₁-C₆-branched alkyl, alkenyl, alkynyl, haloalkyl, alkoxy, cycloalkoxy, haloalkoxy, halo, nitro, cyano, —CH₂-cycloalkyl, —CF₂-cycloalky, —CH(CH₃)-cycloalkyl, —C(═O)heterocycloalkyl, and —C(═O)-morpholine;
wherein substitutions R₁₂, R₁₃, and R₁₄ are only present where valency permits.

18. The compound of claim 17, wherein X₆ in N and X₇ is C.

19. The compound of claim 17, wherein X₆ in C and X₇ is N.

20. The compound of claim 17, wherein R₁₂, R₁₃, and R₁₄ are independently selected from a group consisting of: H, fluoro, chloro, bromo, cyano, CH₃, —CH—(CH₃)₂, —CH₂—CN and —C(═O)-morpholine.

21. The compound of claim 17, wherein R₁₅ and R₁₆ are independently selected from a group consisting of: H, fluoro, chloro, bromo, CH₃, —O—CH₃, —O—CF₃, —CF₃, ethyl, vinyl, or —CH₂—CN.

22. The compound of claim 1 or a pharmaceutically acceptable salt thereof,
wherein the compound is a compound of Formula (V): 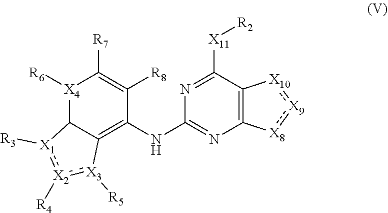
wherein X₈ is N or NH;
X₉ is CH or N,
X₁₀ is NR₁₇, N, or CR₁₇;
X₁₁ is O or NH.
R₁₇ is selected from the group consisting of H, halo, and alkyl.

23. The compound of claim 22, wherein R₁₇ is chloro.

24. The compound of claim 22, wherein R₁₇ is methyl.

25. A compound selected from the group consisting of:

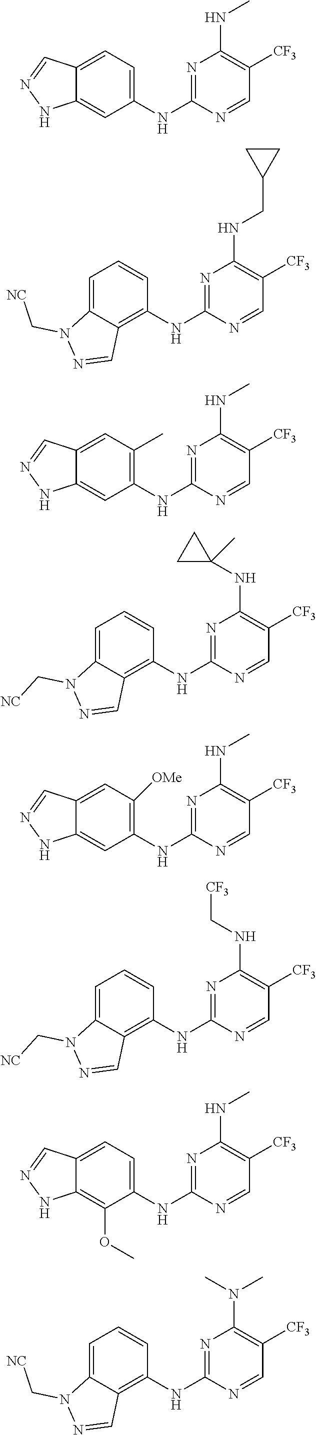

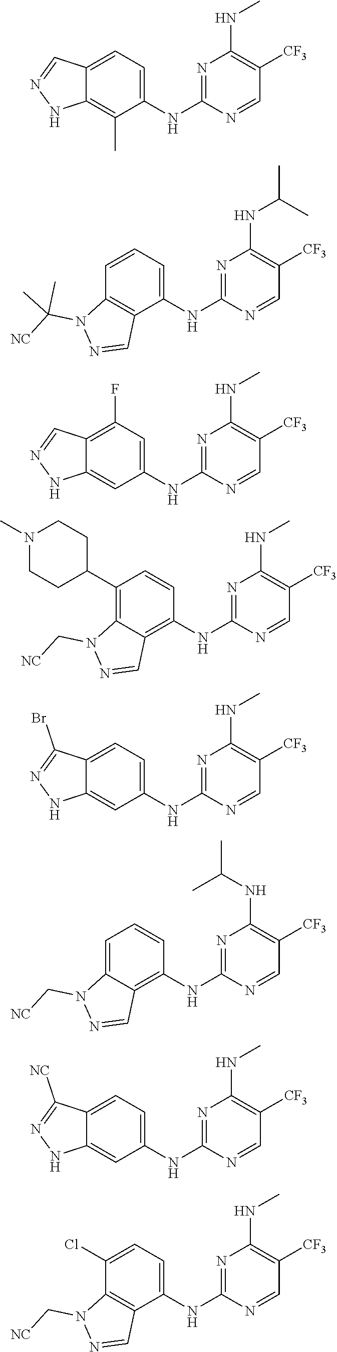

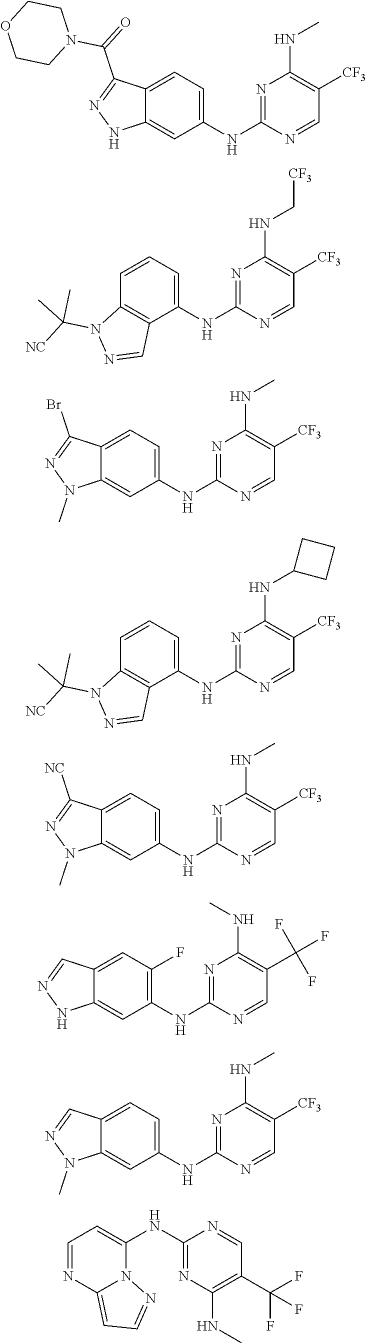

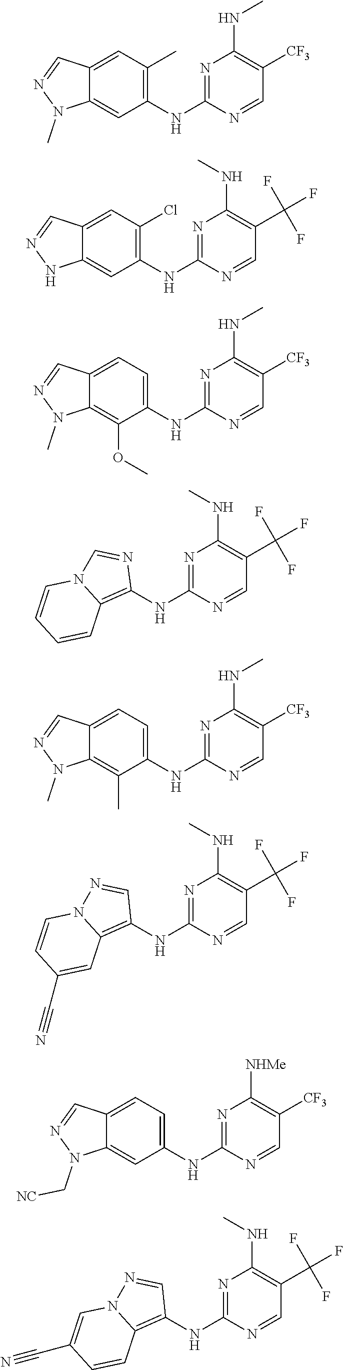

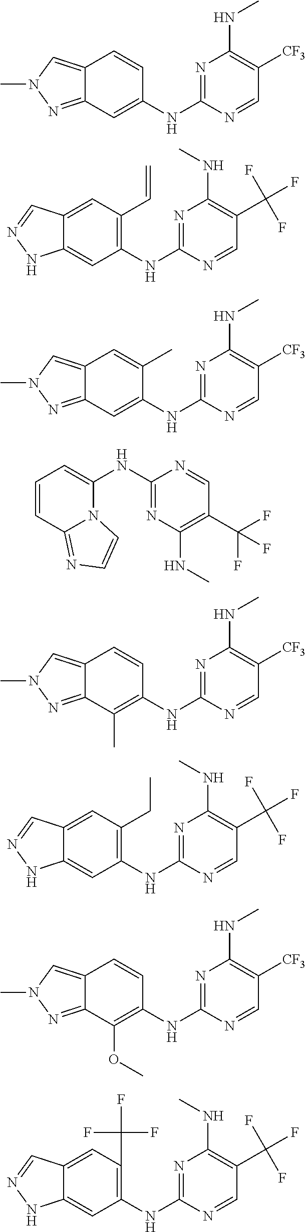

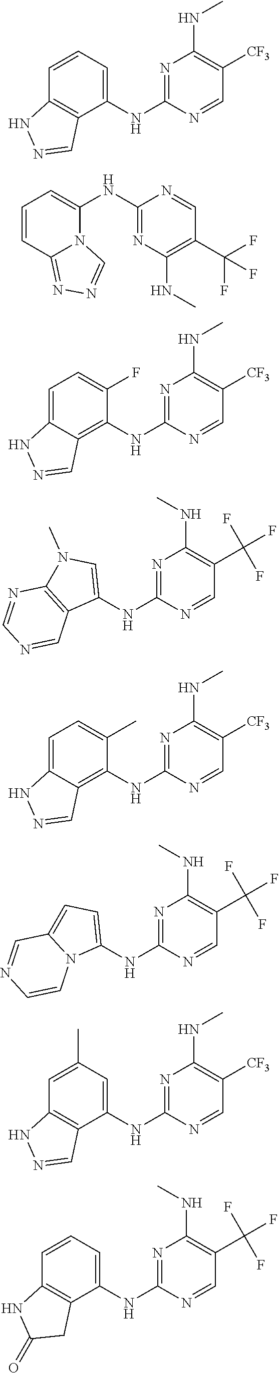

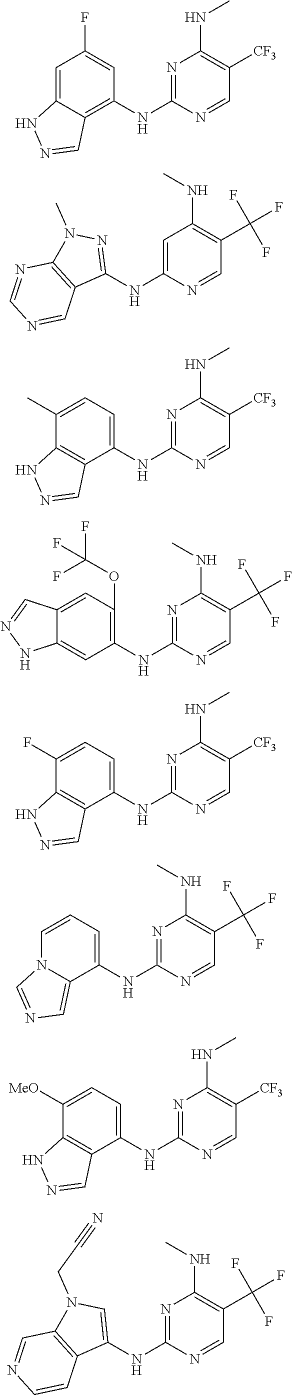

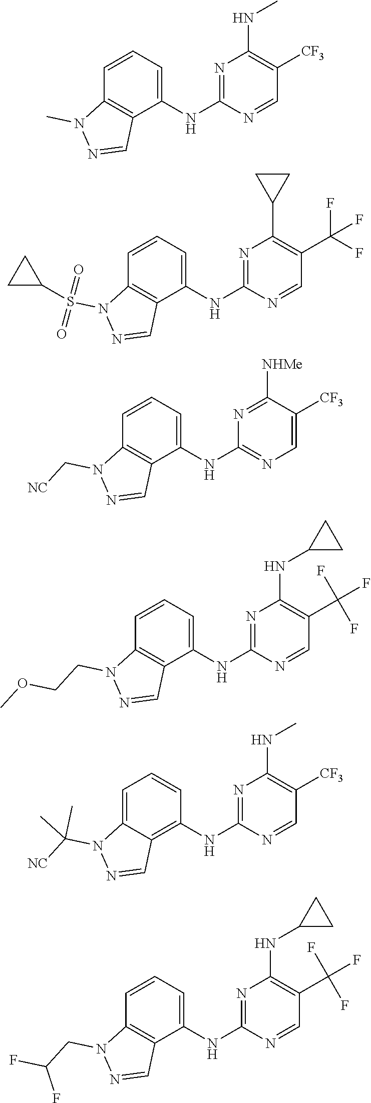 [SMILES: CNc1nc(Nc2cccc3c2cnn3C)ncc1C(F)(F)F ; O=S(=O)(C1CC1)n1ncc2c(Nc3ncc(C(F)(F)F)c(C4CC4)n3)cccc21 ; CNc1nc(Nc2cccc3c2cnn3CC#N)ncc1C(F)(F)F ; COCCn1ncc2c(Nc3ncc(C(F)(F)F)c(NC4CC4)n3)cccc21 ; CNc1nc(Nc2cccc3c2cnn3C(C)(C)C#N)ncc1C(F)(F)F ; FC(F)Cn1ncc2c(Nc3ncc(C(F)(F)F)c(NC4CC4)n3)cccc21]

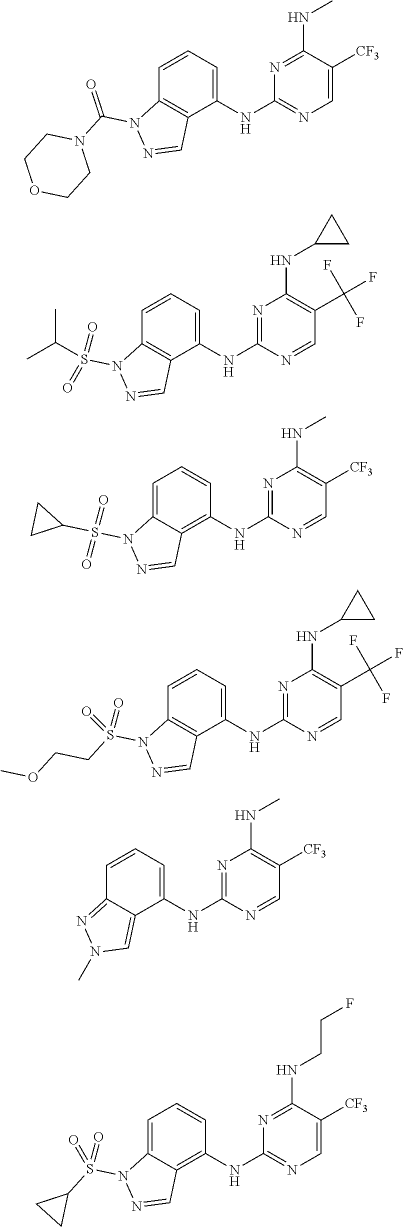 [SMILES: CNc1nc(Nc2cccc3c2cnn3C(=O)N2CCOCC2)ncc1C(F)(F)F ; CC(C)S(=O)(=O)n1ncc2c(Nc3ncc(C(F)(F)F)c(NC4CC4)n3)cccc21 ; CNc1nc(Nc2cccc3c2cnn3S(=O)(=O)C2CC2)ncc1C(F)(F)F ; COCCS(=O)(=O)n1ncc2c(Nc3ncc(C(F)(F)F)c(NC4CC4)n3)cccc21 ; CNc1nc(Nc2cccc3nn(C)cc23)ncc1C(F)(F)F ; O=S(=O)(C1CC1)n1ncc2c(Nc3ncc(C(F)(F)F)c(NCCF)n3)cccc21]

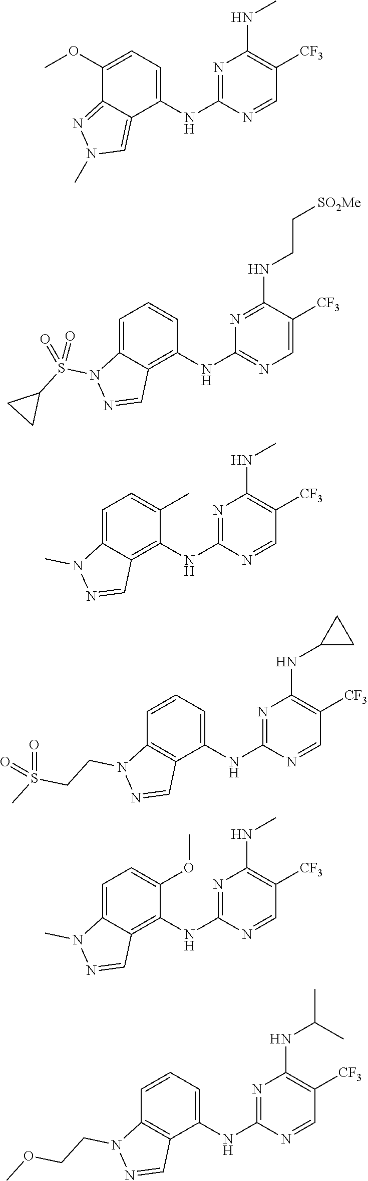

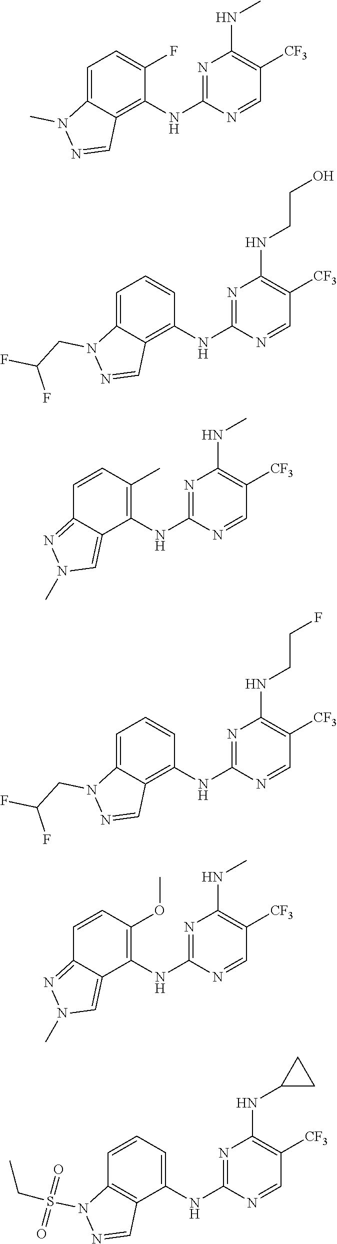 [SMILES: CNc1nc(Nc2c(F)ccc3c2cnn3C)ncc1C(F)(F)F ; OCCNc1nc(Nc2cccc3c2cnn3CC(F)F)ncc1C(F)(F)F ; CNc1nc(Nc2c(C)ccc3nn(C)cc23)ncc1C(F)(F)F ; FCCNc1nc(Nc2cccc3c2cnn3CC(F)F)ncc1C(F)(F)F ; CNc1nc(Nc2c(OC)ccc3nn(C)cc23)ncc1C(F)(F)F ; CCS(=O)(=O)n1ncc2c(Nc3ncc(C(F)(F)F)c(NC4CC4)n3)cccc21]

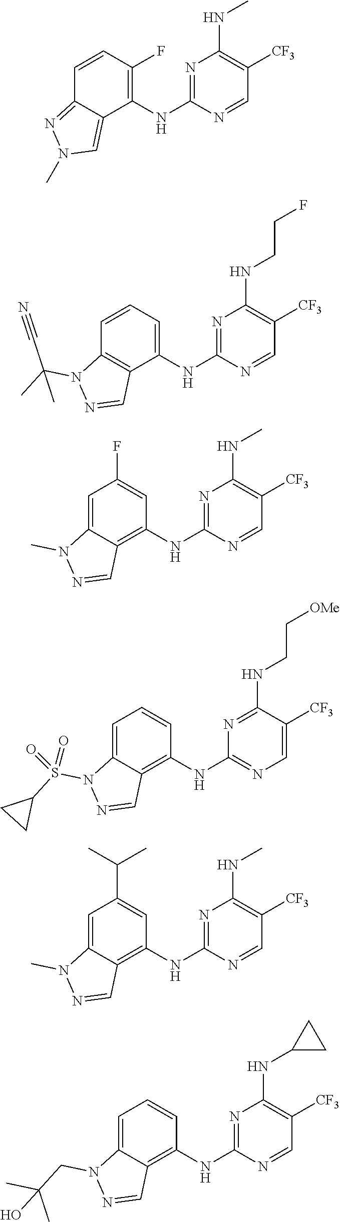

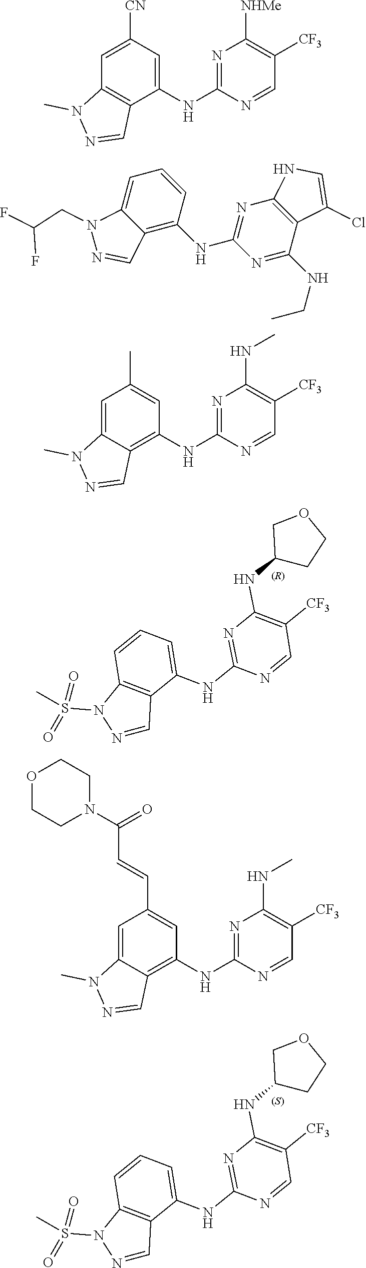

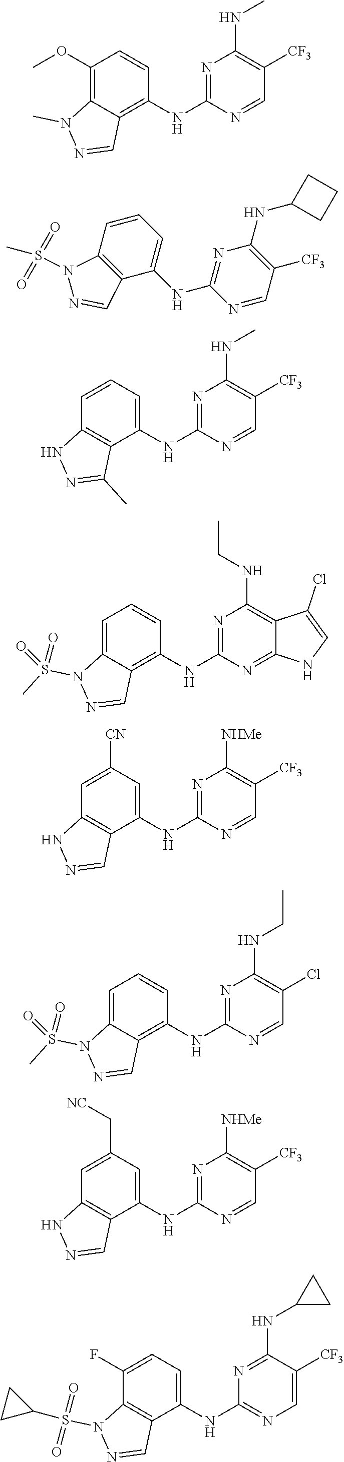

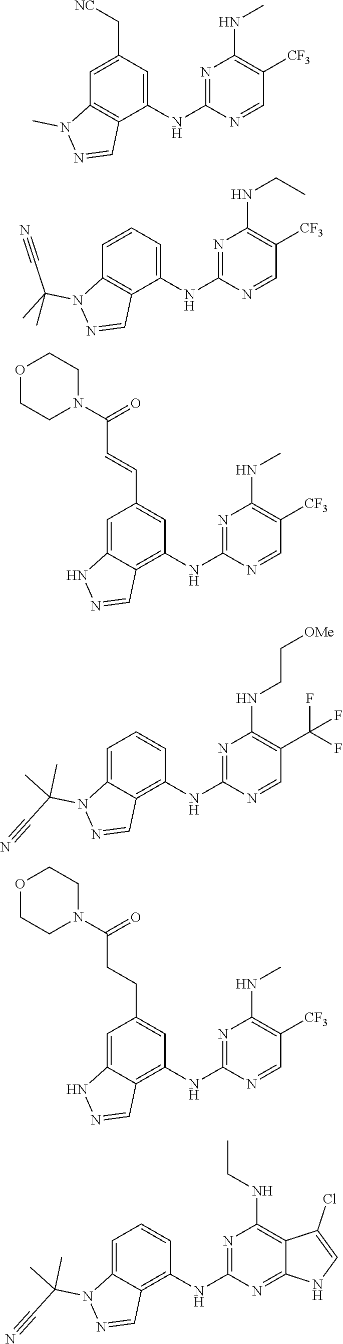

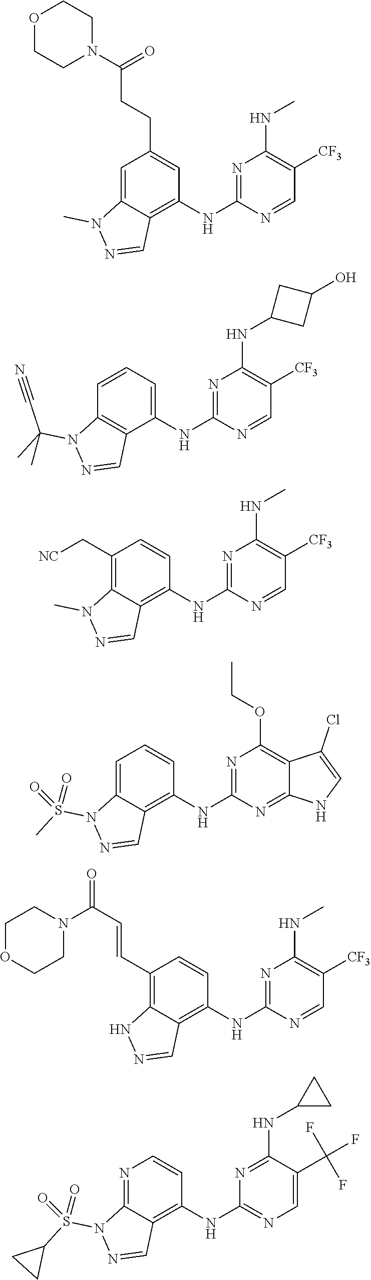

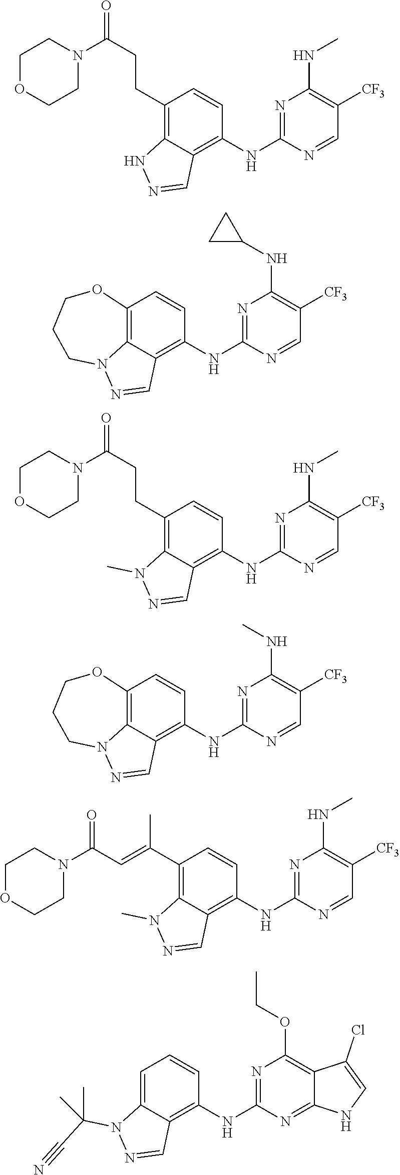

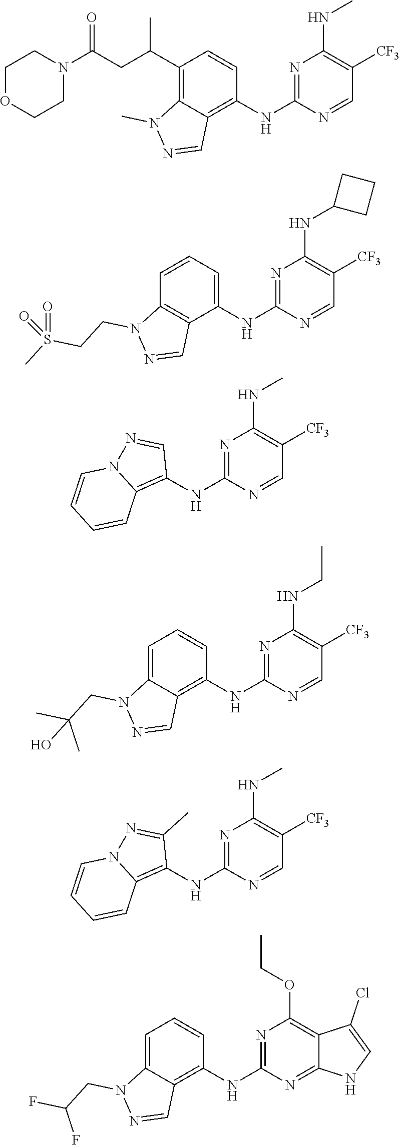 [SMILES: CNc1nc(Nc2ccc(C(C)CC(=O)N3CCOCC3)c3c2cnn3C)ncc1C(F)(F)F ; CS(=O)(=O)CCn1ncc2c(Nc3ncc(C(F)(F)F)c(NC4CCC4)n3)cccc21 ; CNc1nc(Nc2cnn3ccccc23)ncc1C(F)(F)F ; CCNc1nc(Nc2cccc3c2cnn3CC(C)(C)O)ncc1C(F)(F)F ; CNc1nc(Nc2c(C)nn3ccccc23)ncc1C(F)(F)F ; CCOc1nc(Nc2cccc3c2cnn3CC(F)F)nc2[nH]cc(Cl)c12]

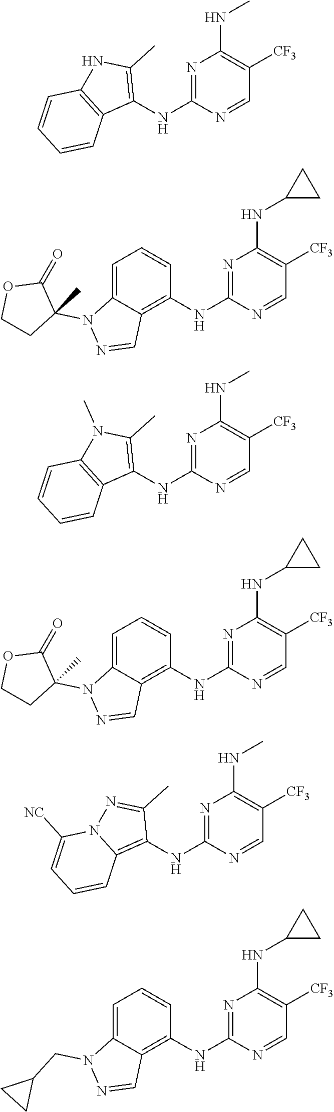 [SMILES: CNc1nc(Nc2c(C)[nH]c3ccccc23)ncc1C(F)(F)F ; C[C@@]1(n2ncc3c(Nc4ncc(C(F)(F)F)c(NC5CC5)n4)cccc32)CCOC1=O ; CNc1nc(Nc2c(C)n(C)c3ccccc23)ncc1C(F)(F)F ; C[C@]1(n2ncc3c(Nc4ncc(C(F)(F)F)c(NC5CC5)n4)cccc32)CCOC1=O ; CNc1nc(Nc2c(C)nn3c(C#N)cccc23)ncc1C(F)(F)F ; FC(F)(F)c1cnc(Nc2cccc3c2cnn3CC2CC2)nc1NC1CC1]

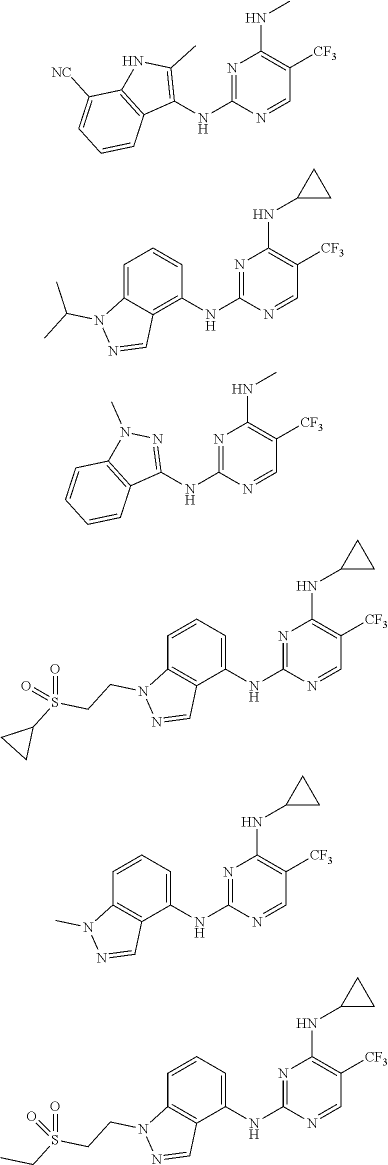 [SMILES: CNc1nc(Nc2c(C)[nH]c3c(C#N)cccc23)ncc1C(F)(F)F ; CC(C)n1ncc2c(Nc3ncc(C(F)(F)F)c(NC4CC4)n3)cccc21 ; CNc1nc(Nc2nn(C)c3ccccc23)ncc1C(F)(F)F ; O=S(=O)(CCn1ncc2c(Nc3ncc(C(F)(F)F)c(NC4CC4)n3)cccc21)C1CC1 ; Cn1ncc2c(Nc3ncc(C(F)(F)F)c(NC4CC4)n3)cccc21 ; CCS(=O)(=O)CCn1ncc2c(Nc3ncc(C(F)(F)F)c(NC4CC4)n3)cccc21]

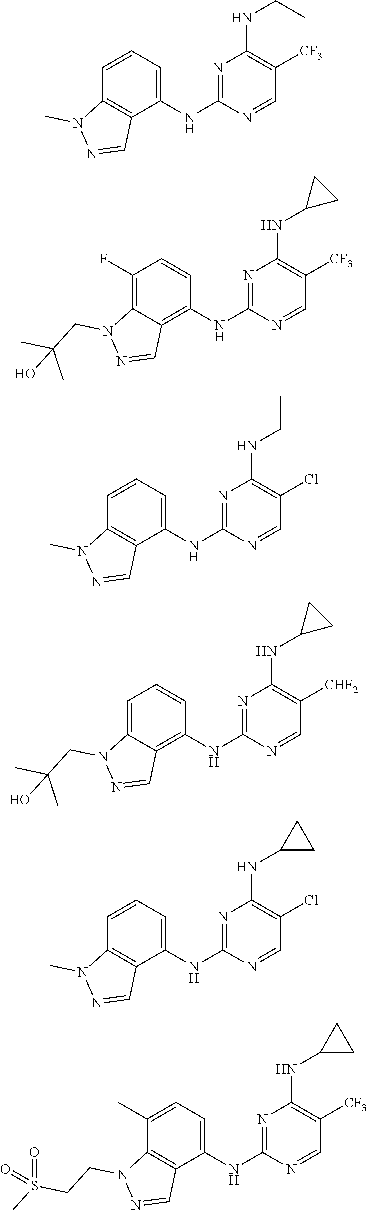 [SMILES: CCNc1nc(Nc2cccc3c2cnn3C)ncc1C(F)(F)F ; CC(C)(O)Cn1ncc2c(Nc3ncc(C(F)(F)F)c(NC4CC4)n3)ccc(F)c21 ; CCNc1nc(Nc2cccc3c2cnn3C)ncc1Cl ; CC(C)(O)Cn1ncc2c(Nc3ncc(C(F)F)c(NC4CC4)n3)cccc21 ; Cn1ncc2c(Nc3ncc(Cl)c(NC4CC4)n3)cccc21 ; Cc1ccc(Nc2ncc(C(F)(F)F)c(NC3CC3)n2)c2cnn(CCS(C)(=O)=O)c12]

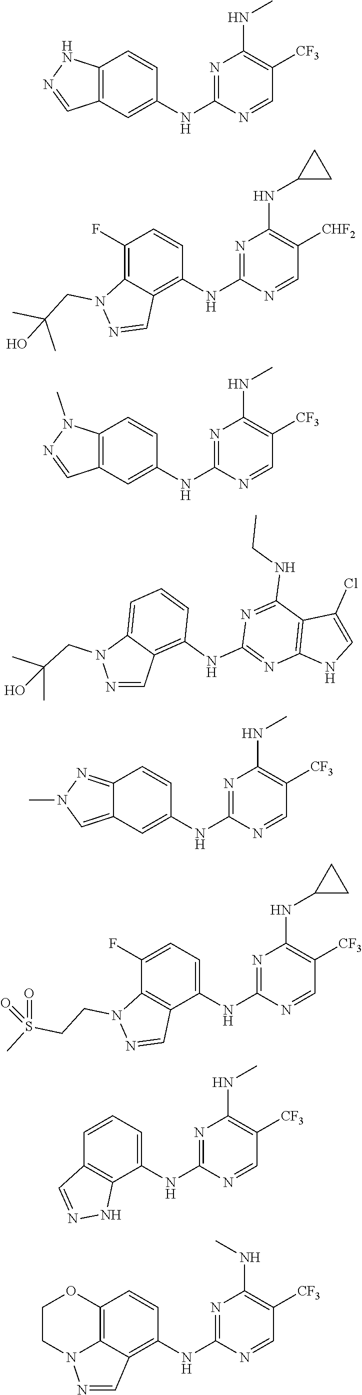

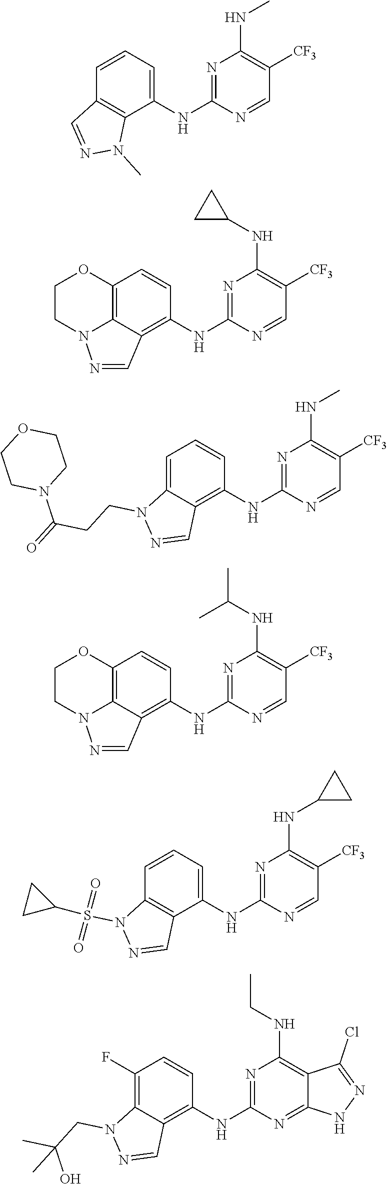 [SMILES: CNc1nc(Nc2cccc3cnn(C)c23)ncc1C(F)(F)F ; FC(F)(F)c1cnc(Nc2ccc3c4c2cnn4CCO3)nc1NC1CC1 ; CNc1nc(Nc2cccc3c2cnn3CCC(=O)N2CCOCC2)ncc1C(F)(F)F ; CC(C)Nc1nc(Nc2ccc3c4c2cnn4CCO3)ncc1C(F)(F)F ; O=S(=O)(C1CC1)n1ncc2c(Nc3ncc(C(F)(F)F)c(NC4CC4)n3)cccc21 ; CCNc1nc(Nc2ccc(F)c3c2cnn3CC(C)(C)O)nc2[nH]nc(Cl)c12]

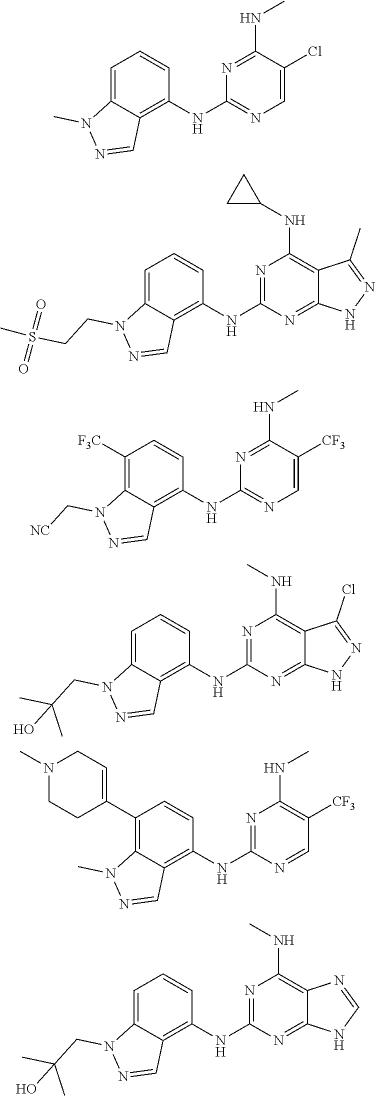

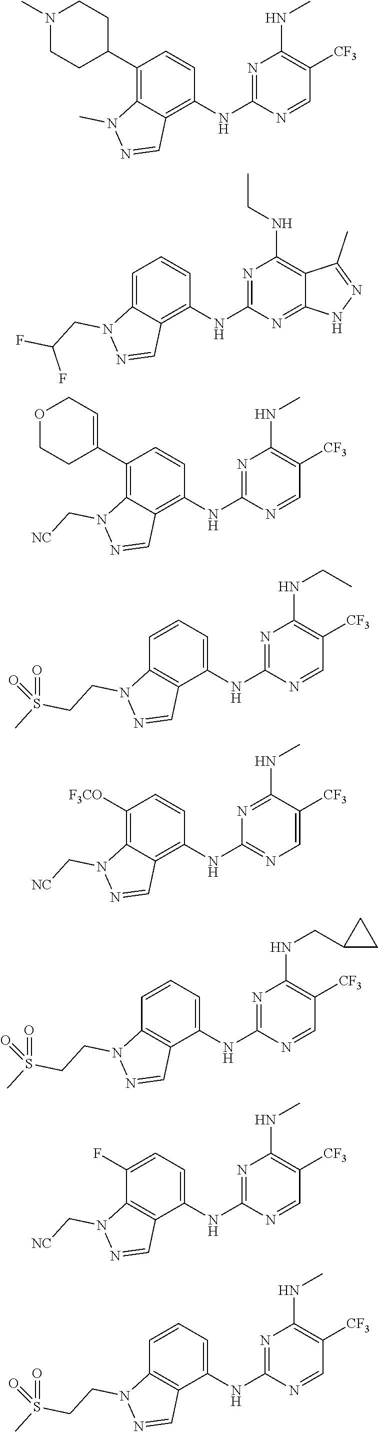

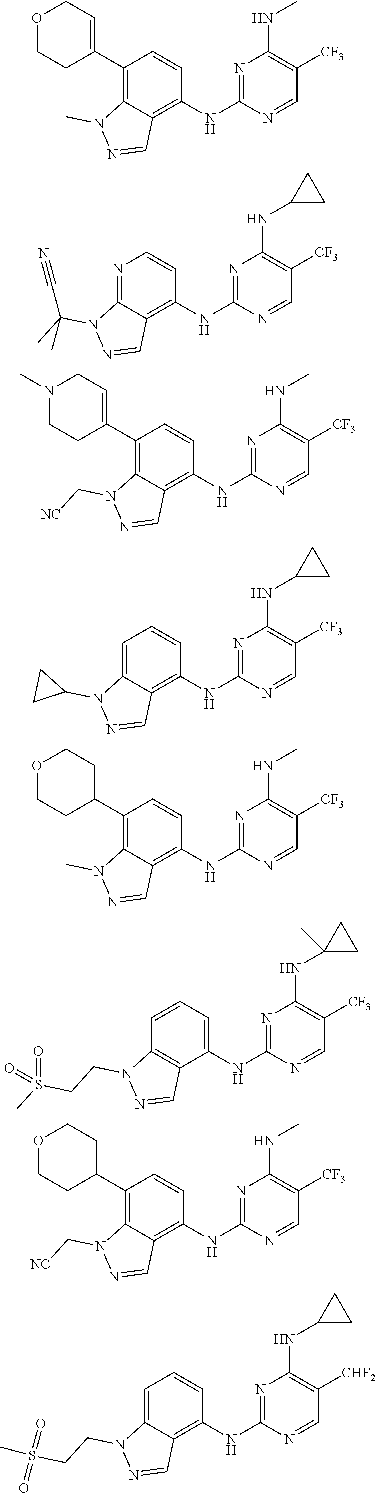

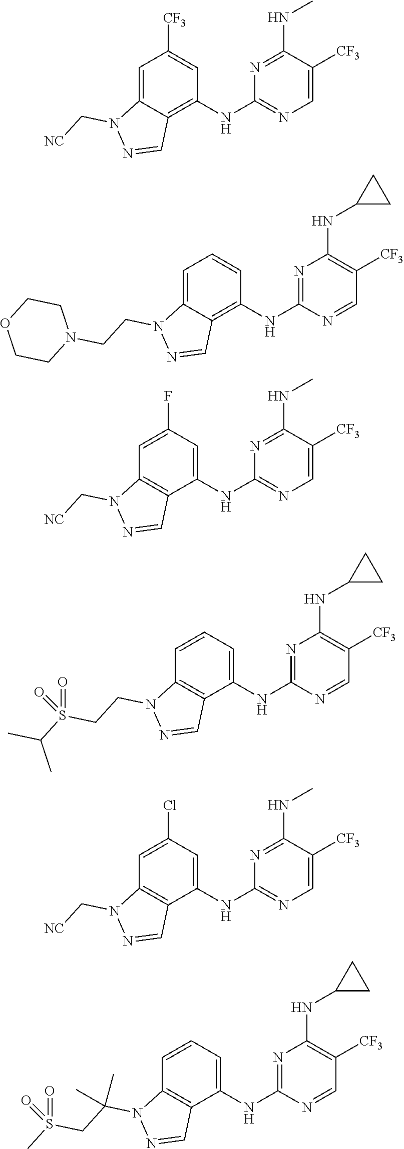

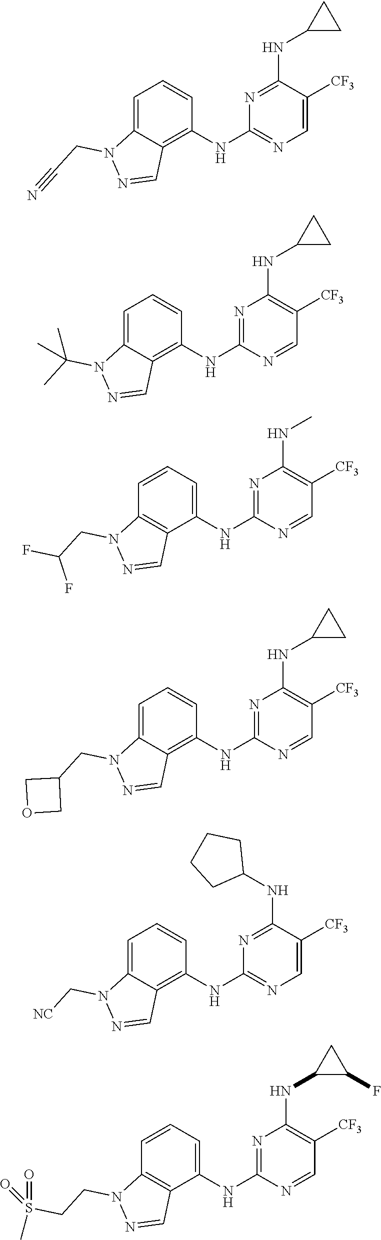

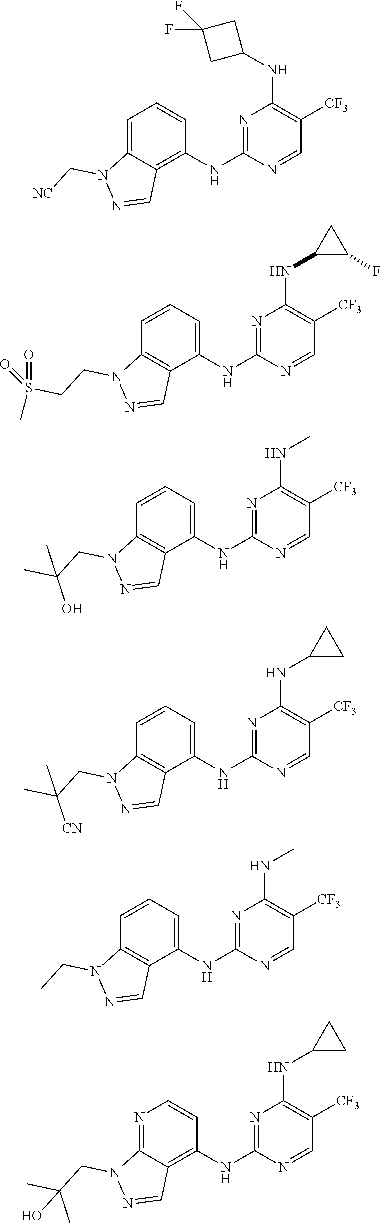

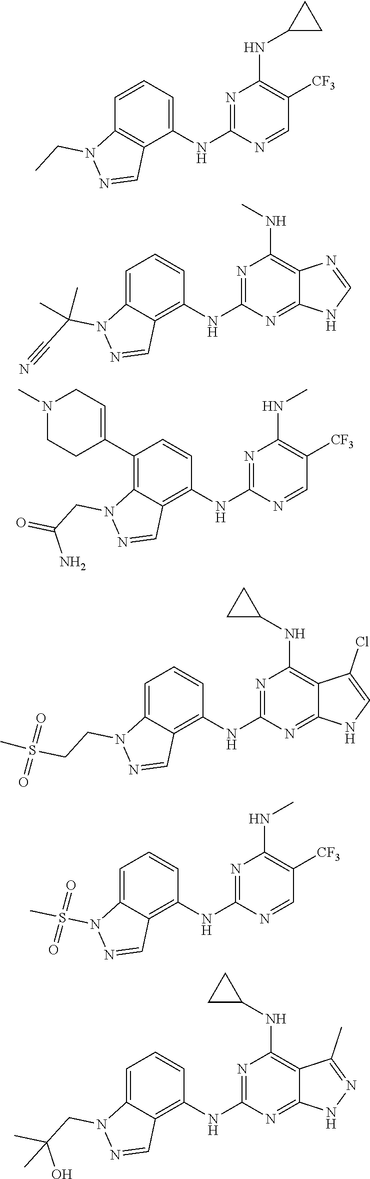

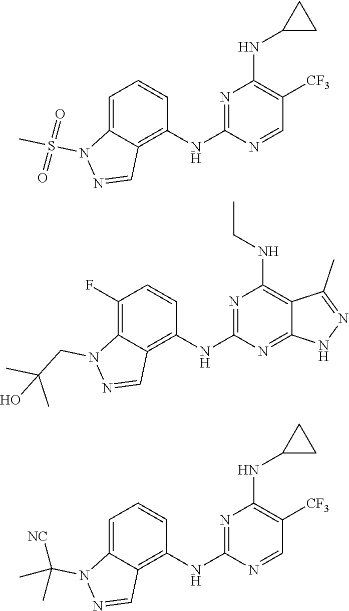 [SMILES: CS(=O)(=O)n1ncc2c(Nc3ncc(C(F)(F)F)c(NC4CC4)n3)cccc21 ; CCNc1nc(Nc2ccc(F)c3c2cnn3CC(C)(C)O)nc2[nH]nc(C)c12 ; CC(C)(C#N)n1ncc2c(Nc3ncc(C(F)(F)F)c(NC4CC4)n3)cccc21]

26. (canceled)

27. A method of treatment of a patient suffering from a neurological condition, wherein the method comprises administering a therapeutically effective amount of compound of Formula (I): 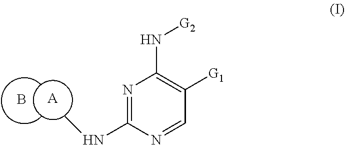
and pharmaceutically acceptable salts thereof,
wherein:
G₁ is CF₃, CHF₂, CH₂F, halogen or cyclopropyl, ethyl, isopropyl;
G₂ is H, substituted or unsubstituted C₁-C₆-alkyl, C₃-C₆-cycloalkyl, C₁-C₆ haloalkyl, C₃-C₆-halocycloalkyl, C₃-C₆-heterocycloalkyl, heteroaryl, —CH₂-cycloalkyl, —CF₂-cycloalkyl, —CH(CH₃)-cycloalkyl, —CH₂-aryl, —CH₂-heterocycloalkyl, —CH₂-heteroaryl, —CF₂-aryl, and —CH(—CH₃)-aryl;
A and B are independently selected from 5 or 6 membered cycloalkyl or aryl rings comprising one or more heteroatoms and one or more substitutions;
wherein both A and B comprise, cumulatively, at least two (2) nitrogen heteroatoms;
wherein A and B are fused at 2 positions;
wherein the one or more substitutions on the A and B rings are independently selected from: H, halo, C₁-C₆-alkyl, branched alkyl, haloalkyl, substituted or unsubstituted alkenyl, substituted or unsubstituted alkynyl, alkoxy, cycloalkoxy, haloalkoxy, alkoxyalkoxy, cyano, —CH₂-cycloalkyl, —CF₂-cycloalky, —(CH₂)₀₋₂C(CH₃)CN, —(CH₂)₀₋₄—CN, —(CH₂)₀₋₄SO₂R′, —CH(CH₃)-cycloalkyl, —CH₂-aryl, —CF₂-aryl, —CH(—CH₃)-aryl, —C(═O)-alkyl, —C(═O)cycloalkyl, —C(═O)—NR′R″, —C(═O)—NH-alkyl, —C(═O)NH₂, hydroxy, —COOH (and ester thereof), alkylsulfonyl, cycloalkylsulfonyl, arylsulfonyl, —S(═O)₂—NR′R″, —NH, —NR′R″, —NR′—S(═O)₂R″, cyanoalkyl, haloalkyl, substituted or unsubstituted alkylsulfonyl, arylsulfonyl, —C(═O)-morpholine, —C(═O)-heterocycles, —C(H)₀₋₁(—CH₃)₁₋₂—OH, —CH₂—C(═O)—NH₂; 3-6 membered heterocycle, any of which may have one or more substituents, wherein the 3-6 membered heterocycle comprises at least one heteroatom independently selected from O, S, and N;
wherein R′ and R″ are independently selected from a group consisting of H, alkyl, substituted or unsubstituted aryl, and heterocycles.
